(19) United States
(12) Patent Application Publication
(10) Pub. No.: US 2024/0263200 A1
(43) Pub. Date: Aug. 8, 2024

(54) PRODUCTION OF SATURATED FATS IN MICROBES

(71) Applicant: Nourish Ingredients Pty Ltd, Mitchell (AU)

(72) Inventors: Anna EL TAHCHY, Moncrieff (AU); Dawar HUSSEIN, Franklin (AU); Bei DONG, Acton (AU); Geoffrey John DUMSDAY, Clayton (AU); Robert Charles DE FEYTER, Monash (AU); Surinder Pal SINGH, Downer (AU); James Robertson PETRIE, Goulburn (AU)

(73) Assignee: Nourish Ingredients Pty Ltd, Mitchell (AU)

(21) Appl. No.: 18/568,751

(22) PCT Filed: Jun. 10, 2022
(86) PCT No.: PCT/AU2022/050580

§ 371 Date: Dec. 8, 2023

(30) Foreign Application Priority Data
Jun. 11, 2021 (AU) 2021901766
Oct. 1, 2021 (AU) 2021903160

(51) Int. Cl. C12P 7/6463; A23L 29/00; C11B 1/10; C12N 1/18; C12N 9/00; C12N 9/10; C12N 15/52; C12N 15/81

(52) U.S. Cl. CPC C12P 7/6463; A23L 29/04; C11B 1/10; C12N 1/185; C12N 9/1029; C12N 9/93; C12N 15/52; C12N 15/815; C12N 2510/00

(57) ABSTRACT

The present invention relates to extracted microbial lipids, microbial cells comprising the lipid, and extracts thereof. The present invention also relates to use of these lipids, cells and extracts in foods, feedstuffs and beverages.

### FIELD OF THE INVENTION

The present invention relates to extracted microbial lipids, microbial cells comprising the lipid, and extracts thereof. The present invention also relates to use of these lipids, cells and extracts in foods, feedstuffs and beverages.

### BACKGROUND OF THE INVENTION

Saturated fatty acids are straight chains of carbon atoms consisting of methylene (CH₂) groups between the end methyl and carboxylic acid groups. The most common saturated fatty acids in foods are lauric acid (C12), palmitic acid (C16) and stearic acid (C18).

Despite health concerns with consuming high levels of certain saturated fatty acids, they nonetheless form an important part of some foods, and processes for the production thereof.

Coconut oil is the most highly saturated naturally occurring fat (typically about 94% saturates). Other ‘saturated’ fats are palm kernel oil (typically, 82% saturates), cocoa butter (typically, 60-64% saturates) and palm oil (typically 51% saturates). Lard and beef tallow are also often considered to be in this category of saturated fats despite typically containing only 40% and 37% saturates, respectively (Talbot, 2011).

There is an increasing need for alternate sources of saturated fatty acids, particularly from non-animal sources, that can be used in foods and food production processes.

### SUMMARY OF THE INVENTION

The present inventors produced new extracted microbial lipid with high levels of saturated fatty acids and low levels of polyunsaturated fatty acids.

Thus, in a first aspect the present invention provides extracted microbial lipid comprising a total fatty acid content which comprises a total saturated fatty acid content of saturated fatty acids (SFA) and a total monounsaturated fatty acid content of monounsaturated fatty acids (MUFA), wherein at least some of the total fatty acid content including at least some of the SFA and at least some of the MUFA is esterified in the form of triacylglycerols (TAG) such that the extracted microbial lipid has a total TAG content, and wherein
(i) the total SFA content of the extracted microbial lipid comprises stearic acid (C18:0), palmitic acid (C16:0), myristic acid (C14:0), arachidic acid (C20:0), behenic acid (C22:0) and lignoceric acid (C24:0), whereby at least 50% by weight of the fatty acids in the total fatty acid content of the extracted microbial lipid are SFA;
(ii) between 20% and 85% by weight of the total fatty acid content is stearic acid;
(iii) the total MUFA content of the extracted microbial lipid comprises oleic acid (C18:1Δ9) and palmitoleic acid (C16:1Δ9), and optionally C16:1Δ7 and/or C17:1;
(iv) the total fatty acid (TFA) content of extracted microbial lipid either lacks polyunsaturated fatty acids (PUFA) or comprises a PUFA content which comprises linoleic acid (C18:2Δ9,12), wherein the PUFA content is less than 5% by weight of the total fatty acid content;
(v) the extracted microbial lipid either comprises polar lipid comprising phospholipid, or lacks polar lipid, and
(vi) the extracted microbial lipid is a solid at 25° C.

In an embodiment, the lipid is solid at one or more or all of 30° C., 35° C., 40° C., 45° C. or 50° C.

In an embodiment, between 20% and 80%, between 20% and 75%, between 20% and 70%, between 20% and 65%, between 20% and 60%, between 20% and 55%, between 20% and 50%, between 20% and 45%, between 25% and 80%, between 25% and 75%, between 25% and 70%, between 25% and 65%, between 25% and 60%, between 25% and 55%, between 25% and 50%, or between 25% and 45% by weight of the TFA content of the lipid or of the TAG content, or both, is stearic acid. In this embodiment, the extracted microbial lipid is preferably an extracted yeast lipid, more preferably an extracted Yarrowia lipid such as a Y. lipolytica lipid.

In an embodiment, the TFA content of the lipid or of the TAG content, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, linoleic acid (LA) by weight, or LA is essentially absent from the TFA of the lipid and/or of the TAG content. In this embodiment, the extracted microbial lipid is preferably an extracted yeast lipid, more preferably an extracted Yarrowia lipid such as a Y. lipolytica lipid.

In an embodiment, the TFA content of the lipid or of the TAG content, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, PUFA by weight, or PUFA are essentially absent from the TFA content of the lipid and/or the TAG content. In this embodiment, the extracted microbial lipid is preferably an extracted yeast lipid, more preferably an extracted Yarrowia lipid such as a Y. lipolytica lipid.

In an embodiment, the TFA content of the lipid, and/or the TFA content of the TAG, comprises at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight of oleic acid. In this embodiment, the extracted microbial lipid is preferably an extracted yeast lipid, more preferably an extracted Yarrowia lipid such as a Y. lipolytica lipid.

In an embodiment, the TFA content of the lipid, and/or the TFA content of the TAG, comprises at least 5%, at least 8%, at least 10% or at least 12% palmitic acid. In embodiments, the palmitic acid content is between 5% and 25%, between 8% and 25%, between 10% and 25%, or between 12% and 25%.

In an embodiment, the lipid was extracted from a microbe comprising at least one genetic modification and which has a greater amount of one or more or all of total SFA content, C18:0 content, C20:0 content and C22:0 content in the TFA content and/or the TAG content of the lipid compared to a corresponding extracted microbial lipid obtained from a corresponding microbe lacking the at least one genetic modification. Exemplary genetic modifications are discussed herein. In this embodiment, the extracted microbial lipid is preferably an extracted yeast lipid, more preferably an extracted Yarrowia lipid such as a Y. lipolytica lipid.

In an embodiment, the TFA content and/or the TAG content of the lipid comprises between 30% and 90%, between 30% and 85%, between 30% and 80%, between 30% and 75%, between 30% and 70%, between 30% and 65%, between 30% and 60%, between 30% and 55%, between 30% and 50%, between 30% and 45%, between 30% and 40%, between 35% and 90%, between 35% and 85%, between 35% and 80%, between 35% and 75%, between 35% and 70%, between 35% and 65%, between 35% and 60%, between 35% and 55%, between 35% and 50%, between 35% and 45%, between 35% and 40%, between 40% and 90%, between 40% and 85%, between 40% and 80%, between 40% and 75%, between 40% and 70%, between 40% and 65%, between 40% and 60%, between 40% and 55%, between 40% and 50%, between 40% and 45%, between 45% and 90%, between 45% and 85%, between 45% and 80%, between 45% and 75%, between 45% and 70%, between 45% and 65%, between 45% and 60%, between 45% and 55%, between 45% and 50%, between 50% and 90%, between 50% and 85%, between 50% and 80%, between 50% and 75%, between 50% and 70%, between 50% and 65%, between 50% and 60%, between 55% and 90%, between 55% and 85%, between 55% and 80%, between 55% and 75%, between 55% and 70%, between 55% and 65%, between 55% and 60%, between 60% and 90%, between 60% and 85%, between 60% and 80%, between 60% and 75%, or between 60% and 70% by weight of SFA. In this embodiment, the extracted microbial lipid is preferably an extracted yeast lipid, more preferably an extracted Yarrowia lipid such as a Y. lipolytica lipid.

In an embodiment, if the total SFA content is at least 60% by weight of the TFA content of the lipid and/or the TFA content of the TAG, the stearic acid content is at least 40% by weight.

In an embodiment, the ratio of total saturated fatty acids comprising 18 carbons or more to total saturated fatty acids comprising 16 carbons or less (L/S-SFA ratio) of the TFA content of the lipid or the TAG content of the lipid, or both, is least about 1.5, is least about 2, is least about 2.5, is least about 3, is least about 4, is least about 5, is least about 6, is least about 7, is least about 8, is least about 9, is least about 10, or between about 3 and about 10. In an embodiment, the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In these embodiments, the extracted microbial lipid is preferably an extracted yeast lipid, more preferably an extracted Yarrowia lipid such as a Y. lipolytica lipid.

In an embodiment, the lipid was extracted from a microbe comprising at least one genetic modification and the L/S-SFA ratio of the TFA content or the TAG content of the lipid, or both, is least about 1.5, is least about 2, is least about 2.5, is least about 3, is least about 4, is least about 5, is least about 6, is least about 7, is least about 8, is least about 9, is least about 10, or between about 3 and about 10, fold greater compared to a corresponding extracted microbial lipid obtained from a corresponding microbe lacking the at least one genetic modification. In an embodiment, the lipid was extracted from a microbe comprising at least one genetic modification and the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, or between 2.5 and 4 fold increased compared to a corresponding extracted microbial lipid obtained from a corresponding microbe lacking the at least one genetic modification. In these embodiments, the extracted microbial lipid is preferably an extracted yeast lipid, more preferably an extracted Yarrowia lipid such as a Y. lipolytica lipid. Exemplary genetic modifications are discussed herein.

In an embodiment, the sum of the contents of C20:0, C22:0 and C24:0 fatty acids is between 5% and 25%, between 5% and 20%, between 5% and 18%, between 5% and 16%, between 5% and 15%, between 5% and 14%, between 5% and 13%, between 5% and 12%, between 5% and 10%, between 6% and 25%, between 6% and 20%, between 6% and 18%, between 6% and 16%, between 6% and 15%, between 6% and 14%, between 6% and 13%, between 6% and 12%, between 6% and 10%, between 7% and 25%, between 7% and 20%, between 7% and 18%, between 7% and 16%, between 7% and 15%, between 7% and 14%, between 7% and 13%, between 7% and 12%, or between 7% and 10%, by weight of the TFA content of the extracted lipid or the TAG content of the extracted lipid, or both. In this embodiment, the extracted microbial lipid is preferably an extracted yeast lipid, more preferably an extracted Yarrowia lipid such as a Y. lipolytica lipid.

In an embodiment, the content of C20:0 fatty acid is between about 1% and about 5%, between about 1% and about 4%, between about 1% and about 3%; between about 2% and about 5%, between about 2% and about 4%, between about 2% and about 3% by weight of the TFA content of the extracted lipid or the TAG content of the extracted lipid, or both.

In an embodiment, the content of C22:0 fatty acid is between about 1% and about 5%, between about 1% and about 4%; between about 2% and about 5%, between about 2% and about 4% by weight of the TFA content of the extracted lipid or the TAG content of the extracted lipid, or both.

In an embodiment, the content of C24:0 fatty acid is between about 1% and about 6%, between about 1% and about 5%, between about 1% and about 4%; between about 2% and about 6%, between about 2% and about 5% or between about 2% and about 4% by weight of the TFA content of the extracted lipid or the TAG content of the extracted lipid, or both.

In another embodiment, the content of C20:0 fatty acid is at least about 1%, at least about 1.5%, at least about 2%, at least about 2.5% or at least about 3%; the content of C22:0 fatty acid is at least about 1%, at least about 1.5%, at least about 2%, at least about 2.5%, at least about 3% or at least about 3.5%; and/or the content of C24:0 fatty acid is at least about 1%, at least about 1.5%, at least about 2%, at least about 2.5%, at least about 3%, at least about 3.5% or at least 4% by weight of the TFA content of the extracted lipid or the TAG content of the extracted lipid, or both.

In an embodiment, (i) between 20% and 55% by weight of the TFA content of the lipid or of the TAG content, or both, is stearic acid, and (ii) less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, is linoleic acid (LA) or total PUFA, or both, by weight, or LA or PUFA or both are essentially absent from the TFA of the lipid and/or of the TAG content. In an embodiment, (i) between 20% and 50% by weight of the TFA content of the lipid or of the TAG content, or both, is stearic acid, and (ii) less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, is linoleic acid (LA) or total PUFA, or both, by weight, or LA or PUFA or both are essentially absent from the TFA of the lipid and/or of the TAG content. In an embodiment, (i) between 20% and 45% by weight of the TFA content of the lipid or of the TAG content, or both, is stearic acid, and (ii) less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, is linoleic acid (LA) or total PUFA, or both, by weight, or LA or PUFA or both are essentially absent from the TFA of the lipid and/or of the TAG content. In these embodiments, the extracted microbial lipid is preferably an extracted yeast lipid, more preferably an extracted Yarrowia lipid such as a Y. lipolytica lipid.

In an embodiment, (i) between 20% and 55% by weight of the TFA content of the lipid or of the TAG content, or both, is stearic acid, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) between 20% and 50% by weight of the TFA content of the lipid or of the TAG content, or both, is stearic acid, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) between 20% and 45% by weight of the TFA content of the lipid or of the TAG content, or both, is stearic acid, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In these embodiments, the extracted microbial lipid is preferably an extracted yeast lipid, more preferably an extracted Yarrowia lipid such as a Y. lipolytica lipid.

In an embodiment, (i) between 20% and 55% by weight of the TFA content of the lipid or of the TAG content, or both, is stearic acid, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) between 20% and 50% by weight of the TFA content of the lipid or of the TAG content, or both, is stearic acid, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) between 20% and 45% by weight of the TFA content of the lipid or of the TAG content, or both, is stearic acid, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In these embodiments, the extracted microbial lipid is preferably an extracted yeast lipid, more preferably an extracted Yarrowia lipid such as a Y. lipolytica lipid.

In an embodiment, the lipid was extracted from a microbe comprising at least one genetic modification and (i) between 20% and 55% by weight of the TFA content of the lipid or of the TAG content, or both, is stearic acid, and (ii) the lipid has a greater amount of one or more or all of total SFA content, C18:0 content, C20:0 content and C22:0 content in the TFA content and/or the TAG content compared to a corresponding extracted microbial lipid obtained from a corresponding microbe lacking the at least one genetic modification. In an embodiment, the lipid was extracted from a microbe comprising at least one genetic modification and (i) between 20% and 50% by weight of the TFA content of the lipid or of the TAG content, or both, is stearic acid, and (ii) the lipid has a greater amount of one or more or all of total SFA content, C18:0 content, C20:0 content and C22:0 content in the TFA content and/or the TAG content compared to a corresponding extracted microbial lipid obtained from a corresponding microbe lacking the at least one genetic modification. In an embodiment, the lipid was extracted from a microbe comprising at least one genetic modification and (i) between 20% and 45% by weight of the TFA content of the lipid or of the TAG content, or both, is stearic acid, and (ii) the lipid has a greater amount of one or more or all of total SFA content, C18:0 content, C20:0 content and C22:0 content in the TFA content and/or the TAG content compared to a corresponding extracted microbial lipid obtained from a corresponding microbe lacking the at least one genetic modification. In these embodiments, the extracted microbial lipid is preferably an extracted yeast lipid, more preferably an extracted Yarrowia lipid such as a Y. lipolytica lipid.

In an embodiment, (i) between 20% and 55% by weight of the TFA content of the lipid or of the TAG content, or both, is stearic acid, and (ii) the content of C24:0 fatty acid in the TFA content of the lipid or of the TAG content, or both, is between about 1% and about 6%, between about 1% and about 5%, between about 1% and about 4%; between about 2% and about 6%, between about 2% and about 5% or between about 2% and about 4% by weight. In an embodiment, (i) between 20% and 50% by weight of the TFA content of the lipid or of the TAG content, or both, is stearic acid, and (ii) the content of C24:0 fatty acid in the TFA content of the lipid or of the TAG content, or both, is between about 1% and about 6%, between about 1% and about 5%, between about 1% and about 4%; between about 2% and about 6%, between about 2% and about 5% or between about 2% and about 4% by weight. In an embodiment, (i) between 20% and 45% by weight of the TFA content of the lipid or of the TAG content, or both, is stearic acid, and (ii) the content of C24:0 fatty acid in the TFA content of the lipid or of the TAG content, or both, is between about 1% and about 6%, between about 1% and about 5%, between about 1% and about 4%; between about 2% and about 6%, between about 2% and about 5% or between about 2% and about 4% by weight. In these embodiments, the extracted microbial lipid is preferably an extracted yeast lipid, more preferably an extracted Yarrowia lipid such as a Y. lipolytica lipid.

In an embodiment, (i) less than 4% by weight of the TFA content of the lipid or of the TAG content, or both, is linoleic acid (LA), and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) less than 3% by weight of the TFA content of the lipid or of the TAG content, or both, is linoleic acid (LA), and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) less than 2% by weight of the TFA content of the lipid or of the TAG content, or both, is linoleic acid (LA), and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) less than 0.2% by weight of the TFA content of the lipid or of the TAG content, or both, is linoleic acid (LA), or the LA is absent from the TFA content of the lipid or of the TAG content, or both, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In these embodiments, the extracted microbial lipid is preferably an extracted yeast lipid, more preferably an extracted Yarrowia lipid such as a Y. lipolytica lipid.

In an embodiment, (i) less than 4% by weight of the TFA content of the lipid or of the TAG content, or both, is linoleic acid (LA), and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) less than 3% by weight of the TFA content of the lipid or of the TAG content, or both, is linoleic acid (LA), and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) less than 2% by weight of the TFA content of the lipid or of the TAG content, or both, is linoleic acid (LA), and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) less than 0.2% by weight of the TFA content of the lipid or of the TAG content, or both, is linoleic acid (LA), or the LA is absent from the TFA content of the lipid or of the TAG content, or both, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In these embodiments, the extracted microbial lipid is preferably an extracted yeast lipid, more preferably an extracted Yarrowia lipid such as a Y. lipolytica lipid.

In an embodiment, (i) less than 4% by weight of the TFA content of the lipid or of the TAG content, or both, is PUFA, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) less than 3% by weight of the TFA content of the lipid or of the TAG content, or both, is PUFA, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) less than 2% by weight of the TFA content of the lipid or of the TAG content, or both, is PUFA, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In an embodiment, (i) less than 0.2% by weight of the TFA content of the lipid or of the TAG content, or both, is PUFA, or the PUFA is absent from the TFA content of the lipid or of the TAG content, or both, and (ii) at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight is oleic acid. In these embodiments, the extracted microbial lipid is preferably an extracted yeast lipid, more preferably an extracted Yarrowia lipid such as a Y. lipolytica lipid.

In an embodiment, (i) less than 4% by weight of the TFA content of the lipid or of the TAG content, or both, is PUFA, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) less than 3% by weight of the TFA content of the lipid or of the TAG content, or both, is PUFA, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) less than 2% by weight of the TFA content of the lipid or of the TAG content, or both, is PUFA, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment, (i) less than 0.2% by weight of the TFA content of the lipid or of the TAG content, or both, is PUFA, or PUFA is absent from the TFA content of the lipid or of the TAG content, or both, and (ii) the L/S-SFA ratio of the TFA content of the lipid or the TAG content of the lipid, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In these embodiments, the extracted microbial lipid is preferably an extracted yeast lipid, more preferably an extracted Yarrowia lipid such as a Y. lipolytica lipid.

In an embodiment, C20:0, C22:0 and C24:0 fatty acids comprise at least 95%, at least 97% or at least 99%, by weight of the fatty acids of the TFA content of the extracted lipid or the TFA content of the TAG of the lipid, or both, which are at least 20 carbons or longer.

In preferred embodiments, the total MUFA content of the extracted microbial lipid, or of the TAG content in the extracted microbial lipid, of the above embodiments comprises (i) oleic acid (C18:1Δ9), palmitoleic acid (C16:1Δ9) and C16:1Δ7, or (ii) oleic acid (C18:1Δ9), palmitoleic acid (C16:1Δ9), C16:1Δ7 and C17:1.

In preferred embodiments, the extracted microbial lipid or the TAG content in the extracted microbial lipid lacks cyclopropane fatty acids.

In an embodiment, the lipid comprises polar lipid, wherein the TFA content of the polar lipid has one or more features as defined herein for the TFA content of the extracted lipid or the TAG content.

In an embodiment, TAG comprises at least 50%, at least 60%, at least 70%, at least 80%, at least 90%, at least 95%, at least 96%, at least 97%, at least 98%, at least 99% or 100% by weight of the total lipid content of the extracted lipid.

In an embodiment, lipid comprises a sterol and/or a sterol ester, preferably ergosterol and/or ergosterol ester. In an embodiment, the extracted microbial lipid comprises at least 1%, at least 2% or at least 3% by weight of ergosterol and/or ergosterol esters. In an embodiment, the extracted microbial lipid comprises between 1% and 10%, between 2% and 10%, or between 3% and 10% by weight of ergosterol and/or ergosterol esters.

In an embodiment, the lipid has a moisture content of less than 20% by weight.

In an embodiment, the lipid has volatile solvent content of less than 10% by weight, less than 7.5% by weight or less than 5% by weight.

In an embodiment, the lipid comprises TAG molecules which comprise a MUFA, preferably oleic acid, esterified at their sn-2 position, wherein the ratio of the number of TAG molecules which comprise a MUFA esterified at the sn-2 position to the number of TAG molecules which comprise a fatty acid other than a MUFA esterified at their sn-2 position (MUFA:other FA ratio at sn-2) in the extracted lipid is less than about 0.50, less than about 0.30, less than about 0.20, less than about 0.10, less than about 0.05, less than about 0.04, less than about 0.03 or less than about 0.02.

In an embodiment, the lipid was extracted from a microbe comprising at least one genetic modification and which comprises polar lipid which comprises phospholipids, wherein at least two, preferably three or all four, of phosphatidylcholine (PC), phosphatidylethanolamine (PE), phosphatidylinositol (PI) and phosphatidylserine (PS) in the extracted lipid have a greater amount of SFA than a corresponding extracted lipid obtained from a corresponding microbe lacking the at least one genetic modification. Exemplary genetic modifications are discussed herein.

In an embodiment, the lipid was extracted from a microbe comprising genetic modifications including:
(a) an exogenous polynucleotide encoding a FATA fatty acyl-thioesterase,
(b) at least one exogenous polynucleotide encoding at least one fatty acid acyltransferase, preferably at least a diacylglycerol acyltransferase (DGAT), and
(c) a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase, most preferably a null mutation of a FAD2 gene, wherein each polynucleotide is operably linked to one or more promoters that are capable of directing expression of said polynucleotides in the microbial cells.

In an embodiment, the at least a DGAT comprises nucleotides having a sequence as set forth as any one of SEQ ID NOs: 144 to 154, or a nucleotide sequence which is at least 70% identical, preferably at least 80% identical, more preferably at least 90%, at least 95% or at least 97% identical, to any one or more of SEQ ID NOs: 144 to 154.

In an embodiment, the FATA fatty acyl-thioesterase comprises nucleotides having a sequence as set forth as any one of SEQ ID NOs: 84 or 86, or a nucleotide sequence which is at least 70% identical, preferably at least 80% identical, more preferably at least 90%, at least 95% or at least 97% identical, to any one or more of SEQ ID NOs: 84 or 86.

In an embodiment, the microbe has a greater amount of one or more or all of total SFA content, C20:0 content, C22:0 content and C24:0 content in the TFA content and/or the TAG content of the lipid compared to a corresponding extracted microbial lipid obtained from a corresponding microbe lacking the at least one genetic modification. In an embodiment, C20:0 content is at least 1%, 1.5% or 2%; C22:0 content is at least 1%, 1.5% or 2%; and C24:0 content is at least 1.5%, 2% or 2.5%. In an embodiment, C20:0 content is between about 1.0% and 4%, C22:0 content is between about 1.0% and 4% and C24:0 content is between about 2% and 5%. In another embodiment, C20:0 content, C22:0 content and C24:0 content include those amounts described above.

In an embodiment, the microbe further comprises an exogenous polynucleotide encoding an acyl-CoA synthetase (ACS), optionally wherein the ACS comprises nucleotides having a sequence as set forth as any one of SEQ ID NOs: 88 to 89, or a nucleotide sequence which is at least 70% identical, preferably at least 80% identical, more preferably at least 90%, at least 95% or at least 97% identical, to any one or more of SEQ ID NOs: 88 to 89.

In another embodiment, the microbe further comprises an exogenous polynucleotide encoding a lysophosphatidic acid acyltransferase (LPAAT), optionally wherein the LPAAT comprises nucleotides having a sequence as set forth in SEQ ID NO: 91, or a nucleotide sequence which is at least 70% identical, preferably at least 80% identical, more preferably at least 90%, at least 95% or at least 97% identical, to SEQ ID NO: 91.

In an embodiment, the extracted microbial lipid is obtained from microbial cells which comprise or consist of eukaryotic cells, fungal cells, bacterial cells or algal cells, living microbial cells, dead microbial cells, or any mixture thereof.

In an embodiment, the microbial cells are one or more or all of (i) suitable for fermentation, (ii) oleaginous cells, (iii) non-oleaginous cells, preferably non-oleaginous cells derived from oleaginous cells by genetic modification, and (iv) heterotrophic cells.

In an embodiment, the microbial cells are yeast cells. Examples of suitable yeast cells include, but are not limited to, Saccharomyces cerevisiae, Yarrowia lipolytica, Pichia pastoris and any mixture thereof.

In an embodiment, the yeast cells are Yarrowia lipolytica.

In an embodiment, the lipid has a weight of at least 5 g, at least 10 g, at least 50 g or at least 100 g.

In an embodiment, the lipid was obtained from microbial cells that have been cultured in the presence of less than 5 g/l, less than 2 g/l, less than 1 g/l of stearate, or have been cultured in the absence of stearate added to the culture medium.

Also provided are microbial cells comprising lipid as defined herein.

In a further aspect, the present invention provides microbial cells, preferably yeast cells, having at least one genetic modification, wherein the microbial cells have (i) an increased saturated fatty acid (SFA) content, an increased content of SFA having at least 18 carbons, an increased content of stearate, an increased content of C20:0 and C22:0 fatty acids, an increased content of C24:0 fatty acid, an increased L/S-SFA ratio, or any combination thereof, in the total fatty acid (TFA) content of the microbial cells or the TFA content of TAG in the microbial cells, or both, and (ii) increased triacylglycerol (TAG) production or accumulation, or both, in each case when compared to corresponding microbial cells lacking the at least one genetic modification and cultured under the same conditions. In preferred embodiments, the cells have (a) an increased saturated fatty acid (SFA) content and (b) an increased triacylglycerol (TAG) production or accumulation, or both, or (a) an increased content of SFA having at least 18 carbons and (b) an increased triacylglycerol (TAG) production or accumulation, or both, or (a) an increased content of stearate and (b) an increased triacylglycerol (TAG) production or accumulation, or both, or (a) an increased content of C20:0 and C22:0 fatty acids and (b) an increased triacylglycerol (TAG) production or accumulation, or both, or (a) an increased content of C24:0 fatty acid and (b) an increased triacylglycerol (TAG) production or accumulation, or both, or (a) an increased L/S-SFA ratio and (b) an increased triacylglycerol (TAG) production or accumulation, or both. In more preferred embodiments, the cells have (a) an increased saturated fatty acid (SFA) content, (b) an increased content of stearate and (c) an increased triacylglycerol (TAG) production or accumulation, or both, or (a) an increased saturated fatty acid (SFA) content, (b) an increased content of C20:0 and C22:0 and (c) an increased triacylglycerol (TAG) production or accumulation, or both, or (a) an increased saturated fatty acid (SFA) content, (b) an increased content of stearate, C20:0 and C22:0 and (c) an increased triacylglycerol (TAG) production or accumulation, or both. In each more preferred embodiment, the lipid content of the cells may also comprise an increased L/S-SFA ratio. In an embodiment, C20:0 content is at least 1%, 1.5% or 2%; C22:0 content is at least 1%, 1.5% or 2%; and C24:0 content is at least 1.5%, 2% or 2.5%. In an embodiment, C20:0 content is between 1.0% and 4%, C22:0 content is between 1.0% and 4% and C24:0 content is between 2% and 5%. In another embodiment, C20:0 content, C22:0 content and C24:0 content include those amounts described above.

In an embodiment, the TFA content of the cells or the TAG content of the cells, or both, has one or more features as defined herein for lipid of the invention for the TFA content of the extracted lipid or the TAG content.

In an embodiment, between 20% and 80%, between 20% and 75%, between 20% and 70%, between 20% and 65%, between 20% and 60%, between 20% and 55%, between 20% and 50%, between 20% and 45%, between 25% and 80%, between 25% and 75%, between 25% and 70%, between 25% and 65%, between 25% and 60%, between 25% and 55%, between 25% and 50%, or between 25% and 45% by weight of the TFA content of the lipid or of the TAG content of the cells, or both, is stearic acid. In this embodiment, the cells are preferably yeast cells, more preferably Yarrowia cells such as Y. lipolytica cells.

In an embodiment, the TFA content of the lipid or of the TAG content of the cells, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, linoleic acid (LA) by weight, or LA is essentially absent from the TFA of the lipid and/or of the TAG content of the cells. In an embodiment, the TFA content of the lipid or of the TAG content of the cells, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, PUFA by weight, or PUFA are essentially absent from the TFA of the lipid and/or of the TAG content of the cells. In these embodiments, the cells are preferably yeast cells, more preferably Yarrowia cells such as Y. lipolytica cells.

In an embodiment, the TFA content of the lipid of the cells, and/or the TFA content of the TAG of the cells, comprises at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight of oleic acid. In this embodiment, the cells are preferably yeast cells, more preferably Yarrowia cells such as Y. lipolytica cells.

In an embodiment, the TFA content of the cells, the TAG content of the cells or the polar lipid of the cells, or any combination thereof, has an increased L/S-SFA ratio when compared to the corresponding microbial cells lacking the at least one genetic modification and cultured under the same conditions. In an embodiment, the L/S-SFA ratio of the TFA content or the TAG content of the lipid of the cells, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In these embodiments, the cells are preferably yeast cells, more preferably Yarrowia cells such as Y. lipolytica cells.

In an embodiment, the L/S-SFA ratio is least about 1.5, is least about 2, is least about 2.5, is least about is least about 3, is least about 4, is least about 5, is least about 6, is least about 7, is least about 8, is least about 9, is least about 10, or between about 3 and about 10, higher than in the TFA content, the TAG content or the polar lipid of the corresponding microbial cells lacking the at least one genetic modification and cultured under the same conditions.

In an embodiment, the microbial cells, preferably yeast cells, comprise at least one genetic modification which is an exogenous polynucleotide(s) encoding:
(a) a FATA fatty acyl-thioesterase,
(b) at least one fatty acid acyltransferase, preferably at least a diacylglycerol acyltransferase (DGAT), or
(c) a FATA and at least one fatty acid acyltransferase, preferably at least a DGAT, wherein each polynucleotide is operably linked to one or more promoters that are capable of directing expression of said polynucleotides in the microbial cells.

In a further aspect, the invention provides a microbial cell, preferably a yeast cell, which comprises at least one genetic modification which is at least one exogenous polynucleotide(s) encoding:
(a) a FATA fatty acyl-thioesterase,
(b) at least one fatty acid acyltransferase, preferably at least a diacylglycerol acyltransferase (DGAT), or
(c) a FATA and at least one fatty acid acyltransferase, preferably at least a DGAT, and
(d) optionally, a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase,
wherein each polynucleotide is operably linked to one or more promoters that are capable of directing expression of said polynucleotides in the microbial cells.

In an embodiment, the genetic modification comprises an exogenous polynucleotide encoding one or more diacylglycerol acyltransferase (DGAT), glycerol-3-phosphate acyltransferase (GPAT) or a phospholipid:diacylglycerol acyltransferase (PDAT) polypeptides, or any combination thereof. In an embodiment comprising one or more DGATs, at least one polynucleotide, or all polynucleotides, in the cell encoding a DGAT comprises nucleotides having a sequence as set forth as any one of SEQ ID NOs: 144 to 154, or a nucleotide sequence which is at least 70% identical, preferably at least 80% identical, more preferably at least 90%, at least 95% or at least 97% identical, to any one or more of SEQ ID NOs: 144 to 154. In an embodiment, at least one polynucleotide, or all polynucleotides that encode a DGAT in the cell, encode a sequence as set forth in one or more of SEQ ID NO: 115 to 125 and differ from one of SEQ ID NOs: 144 to 154 only by codon degeneracy.

In an embodiment, the microbial cell of the invention, preferably a yeast cell, comprises one or more exogenous polynucleotides that each encode a GPAT. In an embodiment, at least one of the GPATs, or all of the GPATs, comprises amino acids having a sequence as set forth as any one SEQ ID NOs: 126 to 138, or an amino acid sequence which is at least 70% identical, preferably at least 80% identical, more preferably at least 90%, at least 95% or at least 97% identical, to any one or more of SEQ ID NOs: 126 to 138.

In an embodiment, at least one polynucleotide, or all polynucleotides, encoding a GPAT comprise nucleotides having a sequence as set forth as any one of SEQ ID NOs: 155 to 167, or a nucleotide sequence which is at least 70% identical, preferably at least 80% identical, more preferably at least 90%, at least 95% or at least 97% identical, to any one or more of SEQ ID NOs: 155 to 167. In an embodiment, at least one polynucleotide, or all polynucleotides that encode a GPAT, encode a sequence as set forth in one or more of SEQ ID NO: 126 to 138 and differ from one of SEQ ID NOs: 155 to 167 only by codon degeneracy.

In an embodiment, the microbial cell of the invention, preferably a yeast cell, comprises one or more exogenous polynucleotides which each encode a phospholipid:diacylglycerol acyltransferase (PDAT). In an embodiment, at least one of the PDATs, or all of the PDATs, comprise amino acids having a sequence as set forth as any one SEQ ID NOs: 139 to 143, or an amino acid sequence which is at least 70% identical, preferably at least 80% identical, more preferably at least 90%, at least 95% or at least 97% identical, to any one or more of SEQ ID NOs: 139 to 143.

In an embodiment, at least one polynucleotide, or all of the polynucleotides, encoding a PDAT comprise nucleotides having a sequence as set forth as any one of SEQ ID NOs: 168 to 172, or a nucleotide sequence which is at least 70% identical, preferably at least 80% identical, more preferably at least 90%, at least 95% or at least 97% identical, to any one or more of SEQ ID NOs: 168 to 172. In an embodiment, at least one polynucleotide, or all polynucleotides that encode a PDAT, encode a sequence as set forth in one or more of SEQ ID NO: 139 to 143 and differ from one of SEQ ID NOs: 168 to 172 only by codon degeneracy.

In an embodiment, the microbial cell, preferably yeast cell, comprises an exogenous polynucleotide encoding a DGAT and an exogenous polynucleotide encoding a GPAT, more than one DGAT and a GPAT, more than one GPAT and a DGAT, or more than one DGAT and more than one GPAT, wherein at least one of the DGATs and/or at least one of the GPATs is as defined above.

In an embodiment, the cell comprises an exogenous polynucleotide encoding a DGAT and an exogenous polynucleotide encoding a PDAT, more than one DGAT and a PDAT, more than one PDAT and a DGAT, or more than one DGAT and more than one PDAT, wherein at least one of the DGATs and/or at least one of the PDATs is as defined above.

In an embodiment, the cell comprises an exogenous polynucleotide encoding a GPAT and an exogenous polynucleotide encoding a PDAT, more than one GPAT and a PDAT, more than one PDAT and a GPAT, or more than one GPAT and more than one PDAT, wherein at least one of the GPATs and/or at least one of the PDATs is as defined above.

In an embodiment, the cell comprises an exogenous polynucleotide encoding a DGAT, an exogenous polynucleotide encoding a GPAT and an exogenous polynucleotide encoding a PDAT, or multiple members of one or more of these.

In an embodiment, the cell comprises exogenous polynucleotides encoding two or more DGATs wherein at least one of the DGATs is as defined above.

In an embodiment, the cell comprises exogenous polynucleotides encoding two or more GPATs wherein at least one of the GPATs is as defined above.

In an embodiment, the cell comprises exogenous polynucleotides encoding two or more PDATs wherein at least one of the PDATs is as defined above.

In a further aspect, the invention provides a microbial cell, preferably a yeast cell, which comprises lipid and at least one genetic modification which is at least one exogenous polynucleotide(s) encoding:
(a) a FATA fatty acyl-thioesterase,
(b) at least one fatty acid acyltransferase, preferably at least a diacylglycerol acyltransferase (DGAT), or
(c) a FATA and at least one fatty acid acyltransferase, preferably at least a DGAT,
wherein each polynucleotide is operably linked to one or more promoters that are capable of directing expression of said polynucleotides in the microbial cells, and wherein
(d) optionally, a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase, most preferably a null mutation of a FAD2 gene, and
(e) between 20% and 80%, between 20% and 75%, between 20% and 70%, between 20% and 65%, between 20% and 60%, between 20% and 55%, between 20% and 50%, between 20% and 45%, between 25% and 80%, between 25% and 75%, between 25% and 70%, between 25% and 65%, between 25% and 60%, between 25% and 55%, between 25% and 50%, or between 25% and 45% by weight of the TFA content of the lipid or of the TAG content of the cells, or both, is stearic acid.

In an embodiment of this aspect, the TFA content of the lipid or of the TAG content of the cells, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, linoleic acid (LA) by weight, or LA is essentially absent from the TFA of the lipid and/or of the TAG content of the cells. In another embodiment of this aspect, the TFA content of the lipid or of the TAG content of the cells, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, PUFA by weight, or PUFA are essentially absent from the TFA of the lipid and/or of the TAG content of the cells. In an embodiment of this aspect, between 20% and 55% by weight of the TFA content of the lipid or of the TAG content of the cells, or both, is stearic acid. In an embodiment of this aspect, between 20% and 50% by weight of the TFA content of the lipid or of the TAG content of the cells, or both, is stearic acid. In an embodiment of this aspect, between 20% and 45% by weight of the TFA content of the lipid or of the TAG content of the cells, or both, is stearic acid.

In a further aspect, the invention provides a microbial cell, preferably a yeast cell, which comprises lipid and at least one genetic modification which is at least one exogenous polynucleotide(s) encoding:
(a) a FATA fatty acyl-thioesterase,
(b) at least one fatty acid acyltransferase, preferably at least a diacylglycerol acyltransferase (DGAT), or
(c) a FATA and at least one fatty acid acyltransferase, preferably at least a DGAT,
wherein each polynucleotide is operably linked to one or more promoters that are capable of directing expression of said polynucleotides in the microbial cells, and wherein
(d) optionally, a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase, most preferably a null mutation of a FAD2 gene, and
(e) the L/S-SFA ratio of the TFA content or the TAG content of the lipid of the cell, or both, is between 1.5 and 10, between 1.5 and 9, between 1.5 and 8, between 1.5 and 7, between 1.5 and 6, between 1.5 and 5, between 1.5 and 4, between 1.75 and 10, between 1.75 and 9, between 1.75 and 8, between 1.75 and 7, between 1.75 and 6, between 1.75 and 5, between 1.75 and 4, between 2 and 10, between 2 and 9, between 2 and 8, between 2 and 7, between 2 and 6, between 2 and 5, between 2 and 4, between 2.5 and 10, between 2.5 and 9, between 2.5 and 8, between 2.5 and 7, between 2.5 and 6, between 2.5 and 5, between 2.5 and 4. In an embodiment of this aspect, between 20% and 55% by weight of the TFA content of the lipid or of the TAG content of the cells, or both, is stearic acid. In an embodiment of this aspect, between 20% and 50% by weight of the TFA content of the lipid or of the TAG content of the cells, or both, is stearic acid. In an embodiment of this aspect, between 20% and 45% by weight of the TFA content of the lipid or of the TAG content of the cells, or both, is stearic acid.

In a further aspect, the invention provides a microbial cell, preferably a yeast cell, which comprises lipid and at least one genetic modification which is at least one exogenous polynucleotide(s) encoding:
(a) a FATA fatty acyl-thioesterase,
(b) at least one fatty acid acyltransferase, preferably at least a diacylglycerol acyltransferase (DGAT), or
(c) a FATA and at least one fatty acid acyltransferase, preferably at least a DGAT, wherein each polynucleotide is operably linked to one or more promoters that are capable of directing expression of said polynucleotides in the microbial cells, and wherein
(d) optionally, a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase, most preferably a null mutation of a FAD2 gene, and
(e) the sum of the contents of C20:0, C22:0 and C24:0 fatty acids in the TFA content or the TAG content of the lipid of the cell, or both, is between 5% and 25%, between 5% and 20%, between 5% and 18%, between 5% and 16%, between 5% and 15%, between 5% and 14%, between 5% and 13%, between 5% and 12%, between 5% and 10%, between 6% and 25%, between 6% and 20%, between 6% and 18%, between 6% and 16%, between 6% and 15%, between 6% and 14%, between 6% and 13%, between 6% and 12%, between 6% and 10%, between 7% and 25%, between 7% and 20%, between 7% and 18%, between 7% and 16%, between 7% and 15%, between 7% and 14%, between 7% and 13%, between 7% and 12%, or between 7% and 10%, by weight; or
(f) the content of C20:0 fatty acid is between about 1% and about 5%, between about 1% and about 4%, between about 1% and about 3%; between about 2% and about 5%, between about 2% and about 4%, between about 2% and about 3% by weight of the TFA content of the extracted lipid or the TAG content of the extracted lipid, or both;
(g) the content of C22:0 fatty acid is between about 1% and about 5%, between about 1% and about 4%; between about 2% and about 5%, between about 2% and about 4% by weight of the TFA content of the extracted lipid or the TAG content of the extracted lipid, or both; and
(h) the content of C24:0 fatty acid is between about 1% and about 6%, between about 1% and about 5%, between about 1% and about 4%; between about 2% and about 6%, between about 2% and about 5% or between about 2% and about 4% by weight of the TFA content of the extracted lipid or the TAG content of the extracted lipid, or both; or
(i) the content of C20:0 fatty acid is at least about 1%, at least about 1.5%, at least about 2%, at least about 2.5% or at least about 3%; the content of C22:0 fatty acid is at least about 1%, at least about 1.5%, at least about 2%, at least about 2.5%, at least about 3% or at least about 3.5%; and the content of C24:0 fatty acid is at least about 1%, at least about 1.5%, at least about 2%, at least about 2.5%, at least about 3%, at least about 3.5% or at least 4% by weight of the TFA content of the extracted lipid or the TAG content of the extracted lipid, or both.

In an embodiment of this aspect, between 20% and 55% by weight of the TFA content of the lipid or of the TAG content of the cells, or both, is stearic acid. In an embodiment of this aspect, between 20% and 50% by weight of the TFA content of the lipid or of the TAG content of the cells, or both, is stearic acid. In an embodiment of this aspect, between 20% and 45% by weight of the TFA content of the lipid or of the TAG content of the cells, or both, is stearic acid.

In embodiments of the above aspects and embodiments, the TFA content of the lipid of the cells, and/or the TFA content of the TAG of the cells, further comprises at least 10%, at least 12%, at least 15%, at least 20%, at least 25%, at least 30%, between 10% and 50%, between 10% and 45%, between 10% and 40%, between 10% and 35%, between 10% and 30%, between 15% and 50%, between 15% and 45%, between 15% and 40%, between 15% and 35%, between 15% and 30%, between 20% and 50%, between 20% and 45%, between 20% and 40%, between 20% and 35%, or between 20% and 30%, by weight of oleic acid.

In embodiments of the above aspects, the at least one acyltransferase, preferably at least the DGAT, has at least equal or greater activity on a steroyl-CoA molecule as a substrate compared to palmitoyl-CoA. The cell may comprise more than one DGAT having this property.

In embodiments of the above aspects, one or more than one or all of the exogenous DGAT(s) comprises amino acids having a sequence set forth as SEQ ID NO: 81, or any one of SEQ ID NOs: 115-125, or an amino acid sequence which is at least 60%, at least 70%, at least 80%, at least 90%, at least 95%, or at least 97% identical to SEQ ID NO: 81 or any one or more of SEQ ID NOs: 115-125.

In embodiments of the above aspects, at least one of the DGATs is a yeast DGA1. In an embodiment, at least one DGA1 comprises amino acids having a sequence set forth as SEQ ID NO: 53, or an amino acid sequence which is at least 60%, at least 70%, at least 80%, at least 90% or at least 95% identical to SEQ ID NO: 53.

In embodiments of the above aspects, the FATA comprises amino acids having a sequence set forth as SEQ ID NO: 83 or SEQ ID NO: 85, or an amino acid sequence which is at least 60%, at least 70%, at least 80%, at least 90% or at least 95% identical to one or both of SEQ ID NO: 83 and SEQ ID NO: 85.

In the above embodiments reciting a DGAT, at least one polynucleotide, or all polynucleotides, in the cell encoding a DGAT may comprise nucleotides having a sequence as set forth as any one of SEQ ID NOs: 144 to 154, or a nucleotide sequence which is at least 70% identical, preferably at least 80% identical, more preferably at least 90%, at least 95% or at least 97% identical, to any one or more of SEQ ID NOs: 144 to 154. In an embodiment, at least one polynucleotide, or all polynucleotides that encode a DGAT in the cell, encode a sequence as set forth in one or more of SEQ ID NO: 115 to 125 and differ from one of SEQ ID NOs: 144 to 154 only by codon degeneracy.

In an embodiment, the microbial cell of the invention, preferably a yeast cell, comprises one or more exogenous polynucleotides that each encode a GPAT. In an embodiment, at least one of the GPATs, or all of the GPATs, comprises amino acids having a sequence as set forth as any one SEQ ID NOs: 126 to 138, or an amino acid sequence which is at least 70% identical, preferably at least 80% identical, more preferably at least 90%, at least 95% or at least 95% identical, to any one or more of SEQ ID NOs: 126 to 138.

In an embodiment, at least one polynucleotide, or all polynucleotides, encoding a GPAT comprise nucleotides having a sequence as set forth as any one of SEQ ID NOs: 155 to 167, or a nucleotide sequence which is at least 70% identical, preferably at least 80% identical, more preferably at least 90%, at least 95% or at least 95% identical, to any one or more of SEQ ID NOs: 155 to 167. In an embodiment, at least one polynucleotide, or all polynucleotides that encode a GPAT, encode a sequence as set forth in one or more of SEQ ID NO: 126 to 138 and differ from one of SEQ ID NOs: 155 to 167 only by codon degeneracy.

In an embodiment, the microbial cell of the invention, preferably a yeast cell, comprises one or more exogenous polynucleotides which each encode a PDAT. In an embodiment, at least one of the PDATs, or all of the PDATs, comprise amino acids having a sequence as set forth as any one SEQ ID NOs: 139 to 143, or an amino acid sequence which is at least 70% identical, preferably at least 80% identical, more preferably at least 90%, at least 95% or at least 95% identical, to any one or more of SEQ ID NOs: 139 to 143.

In an embodiment, at least one polynucleotide, or all of the polynucleotides, encoding a PDAT comprise nucleotides having a sequence as set forth as any one of SEQ ID NOs: 168 to 172, or a nucleotide sequence which is at least 70% identical, preferably at least 80% identical, more preferably at least 90%, at least 95% or at least 95% identical, to any one or more of SEQ ID NOs: 168 to 172. In an embodiment, at least one polynucleotide, or all polynucleotides that encode a PDAT, encode a sequence as set forth in one or more of SEQ ID NO: 139 to 143 and differ from one of SEQ ID NOs: 168 to 172 only by codon degeneracy.

In an embodiment, the microbial cell, preferably yeast cell, comprises an exogenous polynucleotide encoding a diacylglycerol acyltransferase (DGAT) and an exogenous polynucleotide encoding a glycerol-3-phosphate acyltransferase (GPAT), more than one DGAT and a GPAT, more than one GPAT and a DGAT, or more than one DGAT and more than one GPAT, wherein at least one of the DGATs and/or at least one of the GPATs is as defined above.

In an embodiment, the cell comprises an exogenous polynucleotide encoding a DGAT and an exogenous polynucleotide encoding a PDAT, more than one DGAT and a PDAT, more than one PDAT and a DGAT, or more than one DGAT and more than one PDAT, wherein at least one of the DGATs and/or at least one of the PDATs is as defined above.

In an embodiment, the cell comprises an exogenous polynucleotide encoding a GPAT and an exogenous polynucleotide encoding a PDAT, more than one GPAT and a PDAT, more than one PDAT and a GPAT, or more than one GPAT and more than one PDAT, wherein at least one of the GPATs and/or at least one of the PDATs is as defined above.

In an embodiment, the cell comprises an exogenous polynucleotide encoding a DGAT, an exogenous polynucleotide encoding a GPAT and an exogenous polynucleotide encoding a PDAT, or multiple members of one or more of these.

In an embodiment, the cell comprises exogenous polynucleotides encoding two or more DGATs wherein at least one of the DGATs is as defined above.

In an embodiment, the cell comprises exogenous polynucleotides encoding two or more GPATs wherein at least one of the GPATs is as defined above.

In an embodiment, the cell comprises exogenous polynucleotides encoding two or more PDATs wherein at least one of the PDATs is as defined above.

In embodiments of the above aspects, the microbial cells, preferably yeast cells, further comprise an exogenous polynucleotide encoding a lysophosphatidic acid acyltransferase (LPAAT), wherein the polynucleotide is operably linked to one or more promoters that are capable of directing expression of said polynucleotides in the microbial cells. In an embodiment, the LPAAT comprises amino acids having a sequence set forth as SEQ ID NO: 90, or an amino acid sequence which is at least 60%, at least 70%, at least 80%, at least 90% or at least 95% identical to SEQ ID NO: 90.

In embodiments of the above aspects, the microbial cells, preferably yeast cells, further comprise an exogenous polynucleotide encoding a lysophosphatidylcholine acyltransferase (LPCAT), a monoacylglycerol acyltransferase (MGAT), a phospholipase A₂ (PLA₂), a phospholipase C (PLC), a phospholipase D (PLD), a CDP-choline diacylglycerol choline phosphotransferase (CPT), a phosphatidylcholine:diacylglycerol choline phosphotransferase (PDCT), an acyl-CoA synthase (ACS), a fatty acid desaturase, or any combination of two or more thereof.

In embodiments of the above aspects, the microbial cells, preferably yeast cells, more preferably Y. lipolytica cells, comprise a genetic modification resulting in a reduction in endogenous Δ12 desaturase or Δ9 desaturase expression and/or activity, or both Δ12 desaturase and Δ9 desaturase expression and/or activity, preferably a genetic modification of an endogenous gene encoding the Δ12 desaturase or Δ9 desaturase, more preferably a null mutation of an endogenous gene encoding the Δ12 desaturase or a null mutation of an endogenous gene encoding the Δ12 desaturase and a mutation of an endogenous gene encoding a Δ9 desaturase which is not a null mutation. In embodiments of the above aspects, the microbial cells, preferably yeast cells, further comprise an exogenous polynucleotide encoding a silencing RNA molecule which reduces the expression and or activity of a fatty acid desaturase gene, and/or a mutation in a fatty acid desaturase gene which reduces the expression and/or activity of the desaturase gene, preferably a Δ9 desaturase gene or a Δ12 desaturase gene. In a preferred embodiment, the mutation of the Δ9 desaturase gene is not a null mutation. In a preferred embodiment, the mutation of the Δ12 desaturase gene is a null mutation. In an embodiment, the cell further comprises an exogenous polynucleotide encoding a silencing RNA molecule which reduces the expression and or activity of an endogenous DGAT, GPAT or PDAT gene, and/or a mutation in an endogenous DGAT, GPAT or PDAT gene which reduces the expression and/or activity of the desaturase gene.

In preferred embodiments of the above aspects, the genetic modification is a mutation in a gene encoding the endogenous Δ12 desaturase, preferably a null mutation of a FAD2 gene.

In embodiments of the above aspects, the endogenous Δ12 desaturase comprises amino acids having a sequence set forth as SEQ ID NO: 1, or an amino acid sequence which is at least 60%, at least 70%, at least 80%, at least 90% or at least 95% identical to SEQ ID NO: 1.

Thus, in an embodiment, the microbial cell, preferably a yeast cell, comprises genetic modifications including:
(a) an exogenous polynucleotide encoding a FATA fatty acyl-thioesterase,
(b) at least one exogenous polynucleotide encoding at least one fatty acid acyltransferase, preferably at least a diacylglycerol acyltransferase (DGAT), and
(c) a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase, most preferably a null mutation of a FAD2 gene, wherein each polynucleotide is operably linked to one or more promoters that are capable of directing expression of said polynucleotides in the microbial cells.

In an embodiment, the at least a DGAT comprises nucleotides having a sequence as set forth as any one of SEQ ID NOs: 144 to 154, or a nucleotide sequence which is at least 70% identical, preferably at least 80% identical, more preferably at least 90%, at least 95% or at least 97% identical, to any one or more of SEQ ID NOs: 144 to 154.

In an embodiment, the FATA fatty acyl-thioesterase comprises nucleotides having a sequence as set forth as any one of SEQ ID NOs: 84 or 86, or a nucleotide sequence which is at least 70% identical, preferably at least 80% identical, more preferably at least 90%, at least 95% or at least 97% identical, to any one or more of SEQ ID NOs: 84 or 86.

In an embodiment, the microbial cell has a greater amount of one or more or all of total SFA content, C20:0 content, C22:0 content and C24:0 content in the TFA content and/or the TAG content of the lipid compared to a corresponding extracted microbial lipid obtained from a corresponding microbe lacking the at least one genetic modification.

In an embodiment, the content of C20:0 fatty acid is between about 1% and about 5%, between about 1% and about 4%, between about 1% and about 3%; between about 2% and about 5%, between about 2% and about 4%, between about 2% and about 3% by weight of the TFA content of the extracted lipid or the TAG content of the extracted lipid, or both.

In an embodiment, the content of C22:0 fatty acid is between about 1% and about 5%, between about 1% and about 4%; between about 2% and about 5%, between about 2% and about 4% by weight of the TFA content of the extracted lipid or the TAG content of the extracted lipid, or both.

In an embodiment, the content of C24:0 fatty acid is between about 1% and about 6%, between about 1% and about 5%, between about 1% and about 4%; between about 2% and about 6%, between about 2% and about 5% or between about 2% and about 4% by weight of the TFA content of the extracted lipid or the TAG content of the extracted lipid, or both.

In another embodiment, the content of C20:0 fatty acid is at least about 1%, at least about 1.5%, at least about 2%, at least about 2.5% or at least about 3%; the content of C22:0 fatty acid is at least about 1%, at least about 1.5%, at least about 2%, at least about 2.5%, at least about 3% or at least about 3.5%; and the content of C24:0 fatty acid is at least about 1%, at least about 1.5%, at least about 2%, at least about 2.5%, at least about 3%, at least about 3.5% or at least 4% by weight of the TFA content of the extracted lipid or the TAG content of the extracted lipid, or both.

In another embodiment, C20:0 content is between 1.0% and 4%, C22:0 content is between 1.0% and 4% and C24:0 content is between 2% and 5%.

In an embodiment, the microbe further comprises an exogenous polynucleotide encoding an acyl-CoA synthetase (ACS), optionally wherein the ACS comprises nucleotides having a sequence as set forth as any one of SEQ ID NOs: 88 to 89, or a nucleotide sequence which is at least 70% identical, preferably at least 80% identical, more preferably at least 90%, at least 95% or at least 97% identical, to any one or more of SEQ ID NOs: 88 to 89.

In another embodiment, the microbe further comprises an exogenous polynucleotide encoding a lysophosphatidic acid acyltransferase (LPAAT), optionally wherein the LPAAT comprises nucleotides having a sequence as set forth in SEQ ID NO: 91, or a nucleotide sequence which is at least 70% identical, preferably at least 80% identical, more preferably at least 90%, at least 95% or at least 97% identical, to SEQ ID NO: 91.

In embodiments of the above aspects, the microbial cells, preferably yeast cells, comprise a genetic modification resulting in a reduction in gene expression or activity, or both, of an endogenous gene encoding a DGAT that has a preference for a PUFA-CoA as substrate compared to stearoyl-CoA, or a preference for palmitoyl-CoA compared to stearoyl-CoA, or both, preferably a DGA2 gene.

In embodiments of the above aspects, the endogenous DGA2 comprises amino acids having a sequence set forth as SEQ ID NO: 55, or an amino acid sequence which is at least 60%, at least 70%, at least 80%, at least 90% or at least 95% identical to SEQ ID NO: 55.

In embodiments of the above aspects, the microbial cells, preferably yeast cells, have
1) an exogenous polynucleotide(s) encoding a FATA fatty acyl-thioesterase and a genetic modification resulting in a reduction in endogenous Δ12 desaturase gene expression and/or activity,
2) an exogenous polynucleotide(s) encoding a fatty acid acyltransferase, preferably a diacylglycerol acyltransferase (DGAT), and a genetic modification resulting in a reduction in endogenous Δ12 desaturase gene expression and/or activity,
3) exogenous polynucleotides encoding a FATA fatty acyl-thioesterase and a fatty acid acyltransferase, preferably a diacylglycerol acyltransferase (DGAT), and a genetic modification resulting in a reduction in endogenous Δ12 desaturase gene expression and/or activity,
4) an exogenous polynucleotide(s) encoding a FATA fatty acyl-thioesterase and a genetic modification resulting in a reduction in endogenous DGA2 gene expression and/or activity,
5) an exogenous polynucleotide(s) encoding a fatty acid acyltransferase, preferably a diacylglycerol acyltransferase (DGAT), and a genetic modification resulting in a reduction in endogenous DGA2 gene expression and/or activity,
6) exogenous polynucleotides encoding a FATA fatty acyl-thioesterase and a fatty acid acyltransferase, preferably a diacylglycerol acyltransferase (DGAT), and a genetic modification resulting in a reduction in endogenous DGA2 gene expression and/or activity,
7) an exogenous polynucleotide(s) encoding a FATA fatty acyl-thioesterase, a genetic modification resulting in a reduction in endogenous Δ12 desaturase gene expression and/or activity and a genetic modification resulting in a reduction in endogenous DGA2 gene expression and/or activity,
8) an exogenous polynucleotide(s) encoding a fatty acid acyltransferase, preferably a diacylglycerol acyltransferase (DGAT), a genetic modification resulting in a reduction in endogenous Δ12 desaturase gene expression and/or activity, and a genetic modification resulting in a reduction in endogenous DGA2 gene expression and/or activity, or
9) exogenous polynucleotides encoding a FATA fatty acyl-thioesterase and a fatty acid acyltransferase, preferably a diacylglycerol acyltransferase (DGAT), a genetic modification resulting in a reduction in endogenous Δ12 desaturase gene expression and/or activity, and a genetic modification resulting in a reduction in endogenous DGA2 gene expression and/or activity, wherein each polynucleotide is operably linked to one or more promoters that are capable of directing expression of said polynucleotides in the microbial cells.

In embodiments of the above aspects, the microbial cells, preferably yeast cells, comprise or consist of eukaryotic cells, fungal cells, bacterial cells or algal cells, living microbial cells, dead microbial cells, or any mixture thereof.

In embodiments of the above aspects, the microbial cells, preferably yeast cells, are one or more or all of (i) suitable for fermentation, (ii) oleaginous cells, (iii) non-oleaginous cells, preferably non-oleaginous cells derived from oleaginous cells by genetic modification, and (iv) heterotrophic cells.

In embodiments of the above aspects, the microbial cells are yeast cells. Examples of suitable yeast cells include, but are not limited to, Saccharomyces cerevisiae, Yarrowia lipolytica, Pichia pastoris and any mixture thereof.

In embodiments of the above aspects, the yeast cells are Yarrowia lipolytica.

In embodiments of the above aspects, the microbial cells are in a culture medium having less than about 5 g/l, less than about 2 g/l, less than about 1 g/l of stearate, or no added stearate, or have been cultured in said medium.

In another aspect, the present invention provides a microbial cell extract, preferably yeast cell extract, comprising lipid as defined herein or produced from the microbial cells of the invention.

In another aspect, the present invention provides a DNA construct, or a combination of DNA constructs, which encodes one or more of the enzymes defined herein, preferably integrated into the genome of a microbial cell, such as a yeast cell.

In an embodiment, at least one DNA construct, or all DNA constructs, in the cell encoding a DGAT comprises nucleotides having a sequence as set forth as any one of SEQ ID NOs: 144 to 154, or a nucleotide sequence which is at least 70% identical, preferably at least 80% identical, more preferably at least 90%, at least 95% or at least 97% identical, to any one or more of SEQ ID NOs: 144 to 154. In an embodiment, at least one DNA construct, or all DNA constructs that encode a DGAT in the cell, encode a sequence as set forth in one or more of SEQ ID NO: 115 to 125 and differ from one of SEQ ID NOs: 144 to 154 only by codon degeneracy.

In another embodiment, at least one DNA construct, or all DNA constructs, in the cell encoding a FATA fatty acyl-thioesterase comprises nucleotides having a sequence as set forth in any one of SEQ ID NOs: 84 or 86, or a nucleotide sequence which is at least 70% identical, preferably at least 80% identical, more preferably at least 90%, at least 95% or at least 97% identical, to any one or more of SEQ ID NOs: 84 or 86.

In another embodiment, at least one DNA construct, or all DNA constructs, in the cell encoding an acyl-CoA synthetase (ACS) comprises nucleotides having a sequence as set forth in any one of SEQ ID NOs: 88 to 89, or a nucleotide sequence which is at least 70% identical, preferably at least 80% identical, more preferably at least 90%, at least 95% or at least 97% identical, to any one or more of SEQ ID NOs: 88 to 89.

In another embodiment, at least one DNA construct, or all DNA constructs, in the cell encoding a lysophosphatidic acid acyltransferase (LPAAT) comprises nucleotides having a sequence as set forth in SEQ ID NO: 91 or a nucleotide sequence which is at least 70% identical, preferably at least 80% identical, more preferably at least 90%, at least 95% or at least 97% identical, to SEQ ID NO: 91.

In another aspect, the present invention provides a process for producing extracted lipid, comprising
(a) obtaining microbial cells of the invention, and
(b) extracting lipid from the microbial cells,
   so as to thereby produce the extracted lipid. Each of the embodiments of the microbial cells described herein may therefore be used in the process for producing extracted lipid.

In an embodiment, the process further comprises culturing the microbial cells, or a step of treating the cells with an acid prior to step (b).

In an embodiment, the process further comprises culturing the microbial cells, or a step of treating the cells after culturing but before the extraction step, to improve the extraction process, for example treating the cells with an acid prior to step (b).

In an embodiment, the cells are cultured in medium having less than 5 g/l, less than 2 g/l, less than 1 g/l of stearate, or no stearate. Alternatively, the cells are cultured in a medium comprising at least 1 g/l, at least 2 g/l or at least 5 g/l of stearate.

In an embodiment, the step of extracting the lipid comprises exposing the cells to an organic solvent, pressing the cells or treating the cells with microwave irradiation, ultrasonication, high-speed homogenization, high-pressure homogenization, bead beating, autoclaving, thermolysis, or any combination thereof.

In an embodiment, the method further comprises modifying or purifying the lipid after extraction.

In another aspect, the present invention provides a process for culturing microbial cells, the process comprising
(a) obtaining microbial cells of the invention, and
(b) increasing the number of the cells by culturing the cells in a suitable medium.

Each of the embodiments of the microbial cells described herein may therefore be used in the process for culturing the microbial cells.

In another aspect, the present invention provides a process for producing a microbial cell which produces lipid of the invention, the process comprising a step of introducing one or more genetic modifications and/or exogenous polynucleotides as defined herein into a progenitor microbial cell. The process may be used to produce each of the embodiments of the microbial cells of the invention as described herein.

In an embodiment, the process comprises one or more steps of
(i) producing progeny cells from the cell comprising the introduced genetic modifications and/or exogenous polynucleotides,
(ii) mutagenesis of a population of progenitor cells,
(iii) introduction of one or more exogenous polynucleotides whereby the exogenous polynucleotides become integrated into the genome of the microbial cell, preferably into a predetermined location,
(iv) determining the fatty acid composition of the cell or progeny cells thereof, and
(v) selecting a progeny cell which comprises lipid of the invention.

In another aspect, the present invention provides a composition comprising one or more or all of the lipid of the invention, microbial cells of the invention, or the microbial cell extract of the invention. Each of the embodiments of the microbial cells and the extracted lipids described herein may therefore be used to produce the composition. The composition may be used as an ingredient for producing a food or beverage.

In an embodiment, the composition comprises food, feed or beverage ingredient (s) in addition to the lipid of the invention. The lipid or composition of the invention may be used in personal care products such as pharmaceuticals, cosmetics and toiletries.

In an embodiment, the composition comprises one or more fatty acids, esterified or non-esterified, from a source other than the extracted microbial lipid, cell or extract.

In another aspect, the present invention provides a food, feedstuff or beverage comprising an ingredient which is one or more or all of the lipid of the invention, microbial cells of the invention, the microbial cell extract of the invention, or the composition of the invention, and at least one other food, feed or beverage ingredient. Each of the embodiments of the microbial cells and the extracted lipids described herein may therefore be used to produce the food, feedstuff or beverage ingredient of the food, feedstuff or beverage.

In an embodiment, the food, feedstuff or beverage lacks components obtained from an animal.

In an embodiment, the food, feedstuff or beverage ingredient or the food, feedstuff or beverage of the invention is a meat substitute, a fat, oil or dressing, a soup, a noodle product, a stew, a stock, a broth, a sauce, a gravy, a pasta sauce, a tomato product, a dry seasoning mix, a bakery product such as, for example, a bread, bread substitute, pastry, croissant, biscuit, cracker, cake, pizza dough, pie pastry, dry bakery mix, bakery dough, doughnut, a mixed ingredient dish, a snack such as sweet snack, savoury food product or salty snack, a meat substitute or analogue, a dairy product substitute or analogue, or another food product.

In another aspect, the present invention provides a method of producing a food, feedstuff or beverage, the method comprising combining one or more or all of the lipid of the invention, microbial cells of the invention, the microbial cell extract of the invention, or the composition of the invention, with at least one other food, feed or beverage ingredient. Each of the embodiments of the microbial cells and the extracted lipids described herein may therefore be used in the method.

Also provided is the use of one or more or all of the lipid of the invention, microbial cells of the invention, the microbial cell extract of the invention, or the composition of the invention to produce a food, feedstuff or beverage ingredient, or a food, feedstuff or beverage.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Growth curves for S. cerevisiae cultured for up to 7 days in YPD medium.

FIG. 2. Schematic representation for making genetic constructs to introduce inactivating deletions into genes of interest such as microbial FAD2 and URA3. Panel A. DNA synthesis of a 2 kb fragment having 1,000 bp 5′ upstream and 1,000 bp 3′ downstream regions of the gene of interest joined with a SacII site between the two regions. The position of restriction sites and lox sites are indicated by vertical lines. CDS: protein coding region of the gene of interest. B. Amplification of hygromycin (Hph) or nourseothricin (Nat1) antibiotic resistance genes using primers adapted with SacII sites. C. Assembly of genetic construct by insertion of the SacII-ended antibiotic resistance gene cassettes into the DNA fragment of A. Not drawn to scale.

FIG. 3. Schematic representation of construction of genetic constructs for introducing gene deletions into microbes. Panel A. PCR amplification of 5′ upstream and 3′ downstream regions of the gene of interest and ligation together to make a 2 kb fragment. Oligonucleotide primers are shown as small horizontal arrows, restriction enzyme sites and lox sites as vertical lines. CDS: protein coding region of the gene of interest. B. Amplification of hygromycin (Hph) or nourseothricin (Nat1) resistance genes using primers adapted with flanking AsiSI sites. C. Assembly of genetic construct for introduction into microbes such as Y. lipolytica.

FIG. 4. Schematic structure of a phospholipid. One of the hydroxyls can be replaced with different headgroups such as choline, serine or inositol.

FIG. 5. Schematic map of genetic constructs for expression of combinations of candidate PDATs in Y. lipolytica. Each PDAT is under the control of a pTEF promoter and LIP2 gene transcription terminator.

FIG. 6. Schematic map of cloning vectors for introducing candidate acyltransferase genes into Y. lipolytica. Sequence of pNIz0hyg[dga2] provided as SEQ ID NO: 173. Sequence of pNIz0ura[fad2] provided as SEQ ID NO: 174. Sequence of pNIz0nat[lro1] provided as SEQ ID NO: 175. Sequence of pNIz0hyg[pox2] provided as SEQ ID NO: 176.

FIG. 7. (A) Light microscopy of Y. lipolytica strain W29 cells, and (B) transformed Y. lipolytica strain yNI0056 cells, after culturing in a defined medium (M1) to accumulate lipid.

### KEY TO THE SEQUENCE LISTING

SEQ ID NO:1. Amino acid sequence of Y. lipolytica strain W29 FAD2 polypeptide, Accession No. XP_500707.1.

SEQ ID NO:2. Nucleotide sequence of the FAD2 gene of Y. lipolytica strain W29 including upstream and downstream regions. Nucleotides 1-1000 correspond to the 5′ upstream sequence, nucleotides 1001-2260 correspond to the protein coding region for the Δ12 desaturase, and nucleotides 2261-3260 correspond to the 3′ downstream region.

SEQ ID NO:3. Nucleotide sequence of hygromycin resistance selectable marker gene (pTEF-Hyg-tLip2). Nucleotides 1-417 correspond to the TEF promoter (Muller et al., 1998; Accession No. AF054508), nucleotides 418-1443 correspond to the protein coding region for the hygromycin phosphotransferase (Hph) enzyme, and nucleotides 1444-1620 correspond to the polyadenylation region/transcription terminator from the Y. lipolytica strain U6 lipase 2 gene, from Accession No. HM486900 (Darvishi et al., 2011); Nucleotides 20-53 correspond to the loxP site and nucleotides 1569-1598 correspond to the loxR site.

SEQ ID NO:4. Amino acid sequence of hygromycin B phosphotransferase (Hph) encoded by pTEF-Hyg-tLip2.

SEQ ID NO:5. Nucleotide sequence of the nourseothricin resistance selectable marker gene (pTEF-Nat1-tLip2) from GGE368; Accession No. AIC06992, Calvey et al. (2014); Nucleotides 1-418 correspond to the TEF promoter, nucleotides 419-988 correspond to the protein coding region for the nourseothricin acetyltransferase (Nat1) enzyme, and nucleotides 989-1165 correspond to the polyadenylation region/transcription terminator from the Lip2 gene; Nucleotides 20-53 correspond to the loxP site and nucleotides 1114-1143 correspond to the loxR site. 1165 nt.

SEQ ID NO:6. Amino acid sequence of nourseothricin acetyltransferase (Nat1) encoded by the pTEF-Nat1-tLip2 gene.

SEQ ID NO:7. Nucleotide sequence of Y. lipolytica TEF promoter (Muller et al., 1998; Accession No. AF054508).

SEQ ID NOs:8-49. Oligonucleotide primers (see Table 13) SEQ ID NO:50. Amino acid sequence of Y. lipolytica strain URA3 polypeptide, Genbank Accession No. Q12724; 286aa.

SEQ ID NO:51. Nucleotide sequence of a URA3 gene of Y. lipolytica including upstream and downstream regions. Nucleotides 1-1,000 correspond to the 5′ upstream sequence, nucleotides 1,001-1,861 correspond to the protein coding region for the orotidine-5′-phosphate decarboxylase, and nucleotides 1,862-2,861 correspond to the 3′ downstream region.

SEQ ID NO:52. Nucleotide sequence of the DGA1 gene (YALI0E32769p) of Y. lipolytica strain W29, chromosome E, nucleotides 3885857 to 3889401 of Accession No. CR382131.1, including upstream and downstream regions of the DGA1 gene. Nucleotides 1-1,000 correspond to the 5′ upstream sequence, nucleotides 1,001-2,545 correspond to the protein coding region for the DGA1 polypeptide, and nucleotides 2,546-3,545 correspond to the 3′ downstream region; 3,545 nt.

SEQ ID NO:53. Amino acid sequence of DGA1 polypeptide from Y. lipolytica strain W29, encoded by the YALI0E32769p gene, Genbank Accession No. XP_504700.1; 514aa.

SEQ ID NO:54. Nucleotide sequence of the DGA2 gene (YALI0 D07986p) of Y. lipolytica strain W29, chromosome D, nucleotides 1025413 to 1028993 of Accession No. CP017556.1, including upstream and downstream regions of the DGA2 gene. Nucleotides 1-1,000 correspond to the 5′ upstream sequence, nucleotides 1,001-2,581 correspond to the protein coding region for the DGA2 polypeptide, and nucleotides 2,582-3,581 correspond to the 3′ downstream region; 3,581 nt.

SEQ ID NO:55. Amino acid sequence of Y. lipolytica strain W29 DGA2 polypeptide, Genbank Accession No. XP_502557; 526aa.

SEQ ID NO:56. Nucleotide sequence of the LRO1 gene (YALI0E16797p) of Y. lipolytica strain CLIB122, chromosome E, nucleotides 1989950 to 1993896 of Accession No. CR382131.1, including upstream and downstream regions of the LRO1 gene. Nucleotides 1-1000 correspond to the 5′ upstream sequence, nucleotides 1,001-2,947 correspond to the protein coding region for the PDAT, and nucleotides 2,948-3,947 correspond to the 3′ downstream region; 3,947 nt.

SEQ ID NO:57. Amino acid sequence of PDAT from Y. lipolytica strain CLIB122, encoded by the LRO1 gene (YALI0E16797p), Genbank Accession No. XP_504038; 648aa.

SEQ ID NO:58. Nucleotide sequence of the ARE1 gene (YALI0F06578p) of Y. lipolytica strain W29, chromosome F, nucleotides 957751 to 961382 of Accession No. CP028453.1, including upstream and downstream regions of the ARE1 gene. Nucleotides 1-1,000 correspond to the 5′ upstream sequence, nucleotides 1,001-2,632 correspond to the protein coding region for the ASAT, and nucleotides 2,633-3,632 correspond to the 3′ downstream region; 3,632.

SEQ ID NO:59. Amino acid sequence of ASAT from Y. lipolytica strain W29, encoded by the ARE1 gene (YALI0F06578p), Genbank Accession No. XP_505086; 543aa.

SEQ ID NO:60. Nucleotide sequence of the POX1 gene (YALI0E32835 g) of Y. lipolytica strain CLIB122, chromosome E, nucleotides 3897102 to 3899135 of Accession No. CR382131.1, including upstream and downstream regions of the POX1 gene. Nucleotides 1-1,000 correspond to the 5′ upstream sequence, nucleotides 1,001-3,103 correspond to the protein coding region for the POX1, and nucleotides 3,104-4,103 correspond to the 3′ downstream region; 4,103 nt.

SEQ ID NO:61. Amino acid sequence of POX1 from Y. lipolytica strain CLIB122, encoded by YALI0E32835p, GenBank Accession No. XP_504703.1; 677 aa.

SEQ ID NO:62. Nucleotide sequence of the POX2 gene (YALI0F10857 g) of Y. lipolytica strain CLIB122, chromosome F, nucleotides 1449289 to 1451391 of Accession No. CR382132.1, including upstream and downstream regions of the POX2 gene. Nucleotides 1-1,000 correspond to the 5′ upstream sequence, nucleotides 1,001-3,103 correspond to the protein coding region for the POX2, and nucleotides 3,104-4,103 correspond to the 3′ downstream region; 4,103 nt.

SEQ ID NO:63. Amino acid sequence of POX2 from Y. lipolytica strain CLIB122, encoded by YALI0F10857p, GenBank Accession No. XP_505264.1; 700 aa.

SEQ ID NO:64. Nucleotide sequence of the POX3 gene (YALI0 D24750 g) of Y. lipolytica strain CLIB122, chromosome D, nucleotides 3291579 to 3293681 of Accession No. CR382130.1, including upstream and downstream regions of the POX3 gene.

Nucleotides 1-1,000 correspond to the 5′ upstream sequence, nucleotides 1,001-3,103 correspond to the protein coding region for the POX3, and nucleotides 3,104-4,103 correspond to the 3′ downstream region; 4,103 nt.

SEQ ID NO:65. Amino acid sequence of Y. lipolytica strain CLIB122 POX3, encoded by YALI0 D24750p, GenBank Accession No. XP_503244; 700 aa.

SEQ ID NO:66. Nucleotide sequence of the MFE1 gene (YALI0E15378 g) of Y. lipolytica strain CLIB122, chromosome E, nucleotides 1829460 to 1832239 of Accession No. CR382131.1, including upstream and downstream regions of the MFE1 gene. Nucleotides 1-1,000 correspond to the 5′ upstream sequence, nucleotides 1,001-3,706 correspond to the protein coding region for the PDAT, and nucleotides 3,706-4,706 correspond to the 3′ downstream region; 4,706 nt.

SEQ ID NO:67. Amino acid sequence of MFE1 from Y. lipolytica strain CLIB122, encoded by YALI0E15378p, GenBank Accession No. XP_503980; 901 aa.

SEQ ID NO:68. Nucleotide sequence of the PEX10 gene (YALI0C01023 g) of Y. lipolytica strain CLIB122, chromosome C, nucleotides 139718 to 140851 of Accession No. CR382129.1, including upstream and downstream regions of the PEX10 gene. Nucleotides 1-1,000 correspond to the 5′ upstream sequence, nucleotides 1,001-2,134 correspond to the protein coding region for the PEX10, and nucleotides 2,135-3,134 correspond to the 3′ downstream region; 3,134.

SEQ ID NO:69. Amino acid sequence of PEX10 from Y. lipolytica strain CLIB122, encoded by YALI0C01023p, GenBank Accession No. XP_501311; 377 aa.

SEQ ID NO:70. Nucleotide sequence of the SNF1 gene (YALI0 D02101 g) of Y. lipolytica strain CLIB122, chromosome D, nucleotides 236133 to 237872 of Accession No. CR382130.1, including upstream and downstream regions of the SNF1 gene. Nucleotides 1-1,000 correspond to the 5′ upstream sequence, nucleotides 1,001-2,740 correspond to the protein coding region for the SNF1, and nucleotides 2,741-3,740 correspond to the 3′ downstream region; 3,740 nt.

SEQ ID NO:71. Amino acid sequence of SNF1 from Y. lipolytica strain CLIB122, encoded by YALI0 D02101p, GenBank Accession No. XP_502312; 579 aa.

SEQ ID NO:72. Nucleotide sequence of the SPO14 gene (YALI0E18898 g) of Y. lipolytica strain CLIB122, chromosome E, nucleotides 2251884 to 2257373 of Accession No. CR382131.1, including upstream and downstream regions of the SPO14 gene. Nucleotides 1-1,000 correspond to the 5′ upstream sequence, nucleotides 1,001-6,490 correspond to the protein coding region for the SPO14, and nucleotides 6,491-7,490 correspond to the 3′ downstream region; 7,490 nt.

SEQ ID NO:73. Amino acid sequence of SPO14 from Y. lipolytica strain CLIB122, encoded by YALI0E18898p, GenBank Accession No. XP_504124; 1829 aa.

SEQ ID NO:74. Nucleotide sequence of the OPI1 gene (YALI0C14784 g) of Y. lipolytica strain CLIB122, chromosome E, nucleotides 2251884 to 237872 of Accession No. CR382129.1, including upstream and downstream regions of the OPI1 gene. Nucleotides 1-1,000 correspond to the 5′ upstream sequence, nucleotides 1,001-2,863 correspond to the protein coding region for the OPI1, and nucleotides 2,864-3,863 correspond to the 3′ downstream region; 3,863 nt.

SEQ ID NO:75. Amino acid sequence of OPI1 from Y. lipolytica strain CLIB122, encoded by YALI0C14784p, GenBank Accession No. XP_501843; 620 aa.

SEQ ID NO:76. Nucleotide sequence of the POX1 gene (YGL205W; chrVII:108158-110404) of S. cerevisiae including upstream and downstream regions. Nucleotides 1-1,000 correspond to the 5′ upstream sequence, nucleotides 1,001-3,247 correspond to the protein coding region for the acyl-CoA oxidase, and nucleotides 3,248-4,247 correspond to the 3′ downstream region.

SEQ ID NO:77. Amino acid sequence of the POX1 gene product (Accession No. NP_011310.1) of S. cerevisiae strain S288C; 748aa.

SEQ ID NO:78. Nucleotide sequence of the FBAIN promoter from Y. lipolytica (U.S. Pat. No. 8,815,566); 973 nt. The translation start ATG is at nucleotides 803-805, the intron is nucleotides 866-967.

SEQ ID NO:79. Nucleotide sequence of a modified Y. lipolytica FBAIN gene promoter (FBAINm); 927 nt. The region corresponding to nucleotides 805-857 of the FBAIN promoter (SEQ ID NO:78) was deleted to produce FBAINm. The promoter was also modified to provide an optimal translation consensus sequence (CACA; Gasmi et al, 2011) immediately upstream of the ATG start codon, nucleotides 925-927, with removal of the wildtype ATG from FBAIN.

SEQ ID NO:80. Nucleotide sequence of Y. lipolytica GPD gene promoter (U.S. Pat. No. 8,815,566); 971 nt. The translation start codon ATG is at nucleotides 969-971.

SEQ ID NO:81. Amino acid sequence of DGAT1 from M. tetraphylla (Macadamia), NCBI Accession No. KT736302.1; 535aa.

SEQ ID NO:82. Nucleotide sequence of the protein coding region cloned into pAT117, encoding MtDGAT1 from M. tetraphylla, flanked by BsaI sites; 1634 nt.

SEQ ID NO:83. Amino acid sequence of GmFATA1 from G. mangostana (Mangosteen), NCBI Accession No. U92876.1; 352aa.

SEQ ID NO:84. Nucleotide sequence of the protein coding region cloned into pAT066 encoding FATA1 protein from G. mangostana, flanked by BsaI sites. The start codon ATG is at nucleotides 9-11 and the stop codon at nucleotides 1068-107. 1081 nt.

SEQ ID NO:85. Amino acid sequence of GmFATA2 from G. mangostana (Mangosteen), NCBI Accession No. U92877.1; 355aa.

SEQ ID NO:86. Nucleotide sequence of the protein coding region cloned into pAT067 encoding FATA2 from G. mangostana. The start codon ATG is at nucleotides 9-11 and the stop codon at nucleotides 1077-1079. 1090 nt.

SEQ ID NO:87. Amino acid sequence of PcACS-X1 from P. chlororaphis, NCBI Accession No. BAD90933; 545aa.

SEQ ID NO:88. Nucleotide sequence of the protein coding region cloned into pAT136 (PcACS-X1) from P. chlororaphis, flanked by BsaI sites. The start codon ATG is at nucleotides 12-14 and the stop codon at nucleotides 1647-1649. 1660 nt.

SEQ ID NO:89. Nucleotide sequence of the protein coding region cloned into pAT138 (PcACS-X2) from P. chlororaphis, flanked by BsaI sites; 1660 nt.

SEQ ID NO:90. Amino acid sequence of MaLPAAT from Mortierella alpina, NCBI Accession No.; 314aa.

SEQ ID NO:91. Nucleotide sequence of the protein coding region of MtLPAAT from M alpina; 945 nt.

SEQ ID NO:92. Nucleotide sequence of the NotI DNA fragment of pAT207 used to transform Y. lipolytica cells. Nucleotides 1-8 and 9096-9103, NotI restriction enzyme sites; nucleotides 9-308, 5′ portion of Y. lipolytica zeta sequence; nucleotides 365-1643, Y. lipolytica URA3 gene in reverse orientation including promoter of URA3 gene at nucleotides 1398-1621; nucleotides 1903-2261, 3960-4318 and 6595-6967, pTEF promoters; nucleotides 1644-1902, 3483-3959 and 6118-6594, enhancer sequence; nucleotides 2269-3330, protein coding region for GmFATA1; nucleotides 4329-5966, protein coding region for PcACS-X1; nucleotides 6968-8575, protein coding region for MtDGAT1; 3331-3482, 5971-6117 and 8580-8696, lip2 transcription terminators; and nucleotides 8701-9095, 3′ portion of Y. lipolytica zeta sequence.

SEQ ID NO:93. Nucleotide sequence of the NotI DNA fragment of pAT208 used to transform Y. lipolytica cells. Nucleotides 1-8 and 9096-9103, NotI restriction enzyme sites; nucleotides 9-308, 5′ portion of Y. lipolytica zeta sequence; nucleotides 365-1643, Y. lipolytica URA3 gene in reverse orientation including promoter of URA3 gene at nucleotides 1398-1621; nucleotides 1903-2261, 3960-4318 and 6595-6967, pTEF promoters; nucleotides 1644-1902, 3483-3959 and 6118-6594, enhancer sequence; nucleotides 2269-3330, protein coding region for GmFATA1; nucleotides 4329-5966, protein coding region for PcACS-X2; nucleotides 6968-8575, protein coding region for MtDGAT1; 3331-3482, 5971-6117 and 8580-8696, lip2 transcription terminators; and nucleotides 8701-9095, 3′ portion of Y. lipolytica zeta sequence.

SEQ ID NO:94. Nucleotide sequence of the NotI DNA fragment of pAT209 used to transform Y. lipolytica cells. Nucleotides 1-8 and 9105-9112, NotI restriction enzyme sites; nucleotides 9-308, 5′ portion of Y. lipolytica zeta sequence; nucleotides 365-1643, Y. lipolytica URA3 gene in reverse orientation including promoter of URA3 gene at nucleotides 1398-1621; nucleotides 1903-2261, 3969-4327 and 6604-6976, pTEF promoters; nucleotides 1644-1902, 3492-3968 and 6127-6603, enhancer sequence; nucleotides 2269-3330, protein coding region for GmFATA2; nucleotides 4338-5975, protein coding region for PcACS-X1; nucleotides 6977-8584, protein coding region for MtDGAT1; 3340-3491, 5980-6126 and 8589-8705, lip2 transcription terminators; and nucleotides 8710-9104, 3′ portion of Y. lipolytica zeta sequence.

SEQ ID NO:95. Nucleotide sequence of the NotI DNA fragment of pAT210 used to transform Y. lipolytica cells. Nucleotides 1-8 and 9105-9112, NotI restriction enzyme sites; nucleotides 9-308, 5′ portion of Y. lipolytica zeta sequence; nucleotides 365-1643, Y. lipolytica URA3 gene in reverse orientation including promoter of URA3 gene at nucleotides 1398-1621; nucleotides 1903-2261, 3969-4327 and 6604-6976, pTEF promoters; nucleotides 1644-1902, 3492-3968 and 6127-6603, enhancer sequence; nucleotides 2269-3330, protein coding region for GmFATA2; nucleotides 4338-5975, protein coding region for PcACS-X2; nucleotides 6977-8584, protein coding region for MtDGAT1; 3340-3491, 5980-6126 and 8589-8705, lip2 transcription terminators; and nucleotides 8710-9104, 3′ portion of Y. lipolytica zeta sequence.

SEQ ID NO:96. Nucleotide sequence of the NotI DNA fragment of pAT211 used to transform Y. lipolytica cells. Nucleotides 1-8 and 8410-8417, NotI restriction enzyme sites; nucleotides 9-308, 5′ portion of Y. lipolytica zeta sequence; nucleotides 365-1643, Y. lipolytica URA3 gene in reverse orientation including promoter of URA3 gene at nucleotides 1398-1621; nucleotides 1903-2261, 3960-4318 and 5909-6267, pTEF promoters; nucleotides 1644-1902, 3483-3959 and 5432-5908, enhancer sequence; nucleotides 2269-3330, protein coding region for GmFATA1; nucleotides 4333-5280, protein coding region for MaLPAAT; nucleotides 6282-7889, protein coding region for MtDGAT1; 3331-3482, 5281-5431 and 7890-8010, lip2 transcription terminators; and nucleotides 8015-8409, 3′ portion of Y. lipolytica zeta sequence.

SEQ ID NO:97. Nucleotide sequence of the NotI DNA fragment of pAT212 used to transform Y. lipolytica cells. Nucleotides 1-8 and 8419-8426, NotI restriction enzyme sites; nucleotides 9-308, 5′ portion of Y. lipolytica zeta sequence; nucleotides 365-1643, Y. lipolytica URA3 gene in reverse orientation including promoter of URA3 gene at nucleotides 1398-1621; nucleotides 1903-2261, 3960-4318 and 5909-6267, pTEF promoters; nucleotides 1644-1902, 3483-3959 and 5432-5908, enhancer sequence; nucleotides 2272-3339, protein coding region for GmFATA2; nucleotides 4342-5289, protein coding region for MaLPAAT; nucleotides 6291-7898, protein coding region for MtDGAT1; 3340-3491, 5290-5490 and 7899-8019, lip2 transcription terminators; and nucleotides 8024-8418, 3′ portion of Y. lipolytica zeta sequence.

SEQ ID NO:98. Oligonucleotide primer at001

SEQ ID NO:99. Oligonucleotide primer at002.

SEQ ID NO:100. Oligonucleotide primer at085.

SEQ ID NO:101. Oligonucleotide primer at086.

SEQ ID NO:102. Oligonucleotide primer at274.

SEQ ID NO:103. Oligonucleotide primer at275.

SEQ ID NO:104. Oligonucleotide primer at297.

SEQ ID NO:105. Oligonucleotide primer at298.

SEQ ID NO:106. Oligonucleotide primer at299.

SEQ ID NO:107. Oligonucleotide primer at300.

SEQ ID NO:108. Nucleotide sequence of the NotI DNA fragment of pAT091 used to transform Y. lipolytica cells. Nucleotides 1-8 and 4648-4655, NotI restriction enzyme sites; nucleotides 9-311, 5′ portion of Y. lipolytica zeta sequence; nucleotides 365-1643, Y. lipolytica URA3 gene in reverse orientation including promoter of URA3 gene at nucleotides 1398-1621; nucleotides 2080-2479, pTEF promoter; nucleotides 1644-2079, enhancer sequence; nucleotides 2487-4127, protein coding region for PcACS-X1; 4128-4248, lip2 transcription terminator; and nucleotides 4254-4647, 3′ portion of Y. lipolytica zeta sequence.

SEQ ID NO:109. Nucleotide sequence of the NotI DNA fragment of pAT108 used to transform Y. lipolytica cells. Nucleotides 1-8 and 4078-4085, NotI restriction enzyme sites; nucleotides 9-311, 5′ portion of Y. lipolytica zeta sequence; nucleotides 365-1643, Y. lipolytica URA3 gene in reverse orientation including promoter of URA3 gene at nucleotides 1398-1621; nucleotides 2080-2479, pTEF promoter; nucleotides 1644-2079, enhancer sequence; nucleotides 2487-3557, protein coding region for GmFATA2; 3558-3682, lip2 transcription terminator; and nucleotides 3683-4077, 3′ portion of Y. lipolytica zeta sequence.

SEQ ID NO:110. Nucleotide sequence of the NotI DNA fragment of pAT135 used to transform Y. lipolytica cells. Nucleotides 1-8 and 4622-4629, NotI restriction enzyme sites; nucleotides 9-311, 5′ portion of Y. lipolytica zeta sequence; nucleotides 365-1643, Y. lipolytica URA3 gene in reverse orientation including promoter of URA3 gene at nucleotides 1398-1621; nucleotides 2080-2479, pTEF promoter; nucleotides 1644-2079, enhancer sequence; nucleotides 2494-4101, protein coding region for MtDGAT1; 4102-4226, lip2 transcription terminator; and nucleotides 4227-4621, 3′ portion of Y. lipolytica zeta sequence.

SEQ ID NO:111. Nucleotide sequence of the NotI DNA fragment of pAT213 used to transform Y. lipolytica cells. Nucleotides 1-8 and 6714-6721, NotI restriction enzyme sites; nucleotides 9-311, 5′ portion of Y. lipolytica zeta sequence; nucleotides 365-1643, Y. lipolytica URA3 gene in reverse orientation including promoter of URA3 gene at nucleotides 1398-1621; nucleotides 2080-2479 and 4146-4546, pTEF promoters; nucleotides 1644-2079 and 3710-4145, enhancer sequences; nucleotides 2487-3557, protein coding region for GmFATA2; nucleotides 4556-6193, protein coding region for PcACS-X1; 3558-3709 and 6194-6318, lip2 transcription terminators; and nucleotides 6319-6713, 3′ portion of Y. lipolytica zeta sequence.

SEQ ID NO:112. Nucleotide sequence of the NotI DNA fragment of pAT214 used to transform Y. lipolytica cells. Nucleotides 1-8 and 6688-6695, NotI restriction enzyme sites; nucleotides 9-311, 5′ portion of Y. lipolytica zeta sequence; nucleotides 365-1643, Y. lipolytica URA3 gene in reverse orientation including promoter of URA3 gene at nucleotides 1398-1621; nucleotides 2080-2479 and 4146-4546, pTEF promoters; nucleotides 1644-2079 and 3710-4145, enhancer sequences; nucleotides 2487-3557, protein coding region for GmFATA2; nucleotides 4560-6167, protein coding region for MtDGAT1; 3558-3709 and 6168-6292, lip2 transcription terminators; and nucleotides 6293-6687, 3′ portion of Y. lipolytica zeta sequence.

SEQ ID NO:113. Nucleotide sequence of the NotI DNA fragment of pAT215 used to transform Y. lipolytica cells. Nucleotides 1-8 and 7258-7265, NotI restriction enzyme sites; nucleotides 9-311, 5′ portion of Y. lipolytica zeta sequence; nucleotides 365-1643, Y. lipolytica URA3 gene in reverse orientation including promoter of URA3 gene at nucleotides 1398-1621; nucleotides 2080-2479 and 4716-5116, pTEF promoters; nucleotides 1644-2079 and 4280-4715, enhancer sequences; nucleotides 2487-4127, protein coding region for PcACS-X1; nucleotides 5130-6737, protein coding region for MtDGAT1; 4128-4279 and 6738-6862, lip2 transcription terminators; and nucleotides 6863-7257, 3′ portion of Y. lipolytica zeta sequence.

SEQ ID NO:114. Nucleotide sequence of the NotI DNA fragment of pAT216 used to transform Y. lipolytica cells. Nucleotides 1-8 and 9260-9267, NotI restriction enzyme sites; nucleotides 9-311, 5′ portion of Y. lipolytica zeta sequence; nucleotides 365-1643, Y. lipolytica URA3 gene in reverse orientation including promoter of URA3 gene at nucleotides 1398-1621; nucleotides 2080-2479, 4146-4545 and 6781-7181, pTEF promoters; nucleotides 1644-2079, 3710-4145 and 6345-6780, enhancer sequence; nucleotides 2487-3557, protein coding region for GmFATA2; nucleotides 4556-6193, protein coding region for PcACS-X1; nucleotides 7195-8739, protein coding region for YlDGA1; 3558-3709, 6194-6344 and 8740-8864, hp2 transcription terminators; and nucleotides 8865-9259, 3′ portion of Y. lipolytica zeta sequence.

SEQ ID NO: 115—Amino acid sequence of TcDGAT1 from T. cacao.

SEQ ID NO: 116—Amino acid sequence of TcDGAT2 from T. cacao.

SEQ ID NO: 117—Amino acid sequence of TcDGAT3 from T. cacao.

SEQ ID NO: 118—Amino acid sequence of TcDGAT4 from T. cacao.

SEQ ID NO: 119—Amino acid sequence of TcDGAT5 from T. cacao.

SEQ ID NO: 120—Amino acid sequence of TcDGAT6 from T. cacao.

SEQ ID NO: 121—Amino acid sequence of TcDGAT7 from T. cacao.

SEQ ID NO: 122—Amino acid sequence of TcDGAT8 from T. cacao.

SEQ ID NO: 123—Amino acid sequence of TcDGAT9 from T. cacao.

SEQ ID NO: 124—Amino acid sequence of TcDGAT10 from T. cacao.

SEQ ID NO: 125—Amino acid sequence of TcDGAT11 from T. cacao.

SEQ ID NO: 126—Amino acid sequence of TcGPAT1 from T. cacao.

SEQ ID NO: 127—Amino acid sequence of TcGPAT2 from T. cacao.

SEQ ID NO: 128—Amino acid sequence of TcGPAT3 from T. cacao.

SEQ ID NO: 129—Amino acid sequence of TcGPAT4 from T. cacao.

SEQ ID NO: 130—Amino acid sequence of TcGPAT5 from T. cacao.

SEQ ID NO: 131—Amino acid sequence of TcGPAT6 from T. cacao.

SEQ ID NO: 132—Amino acid sequence of TcGPAT7 from T. cacao.

SEQ ID NO: 133—Amino acid sequence of TcGPAT8 from T. cacao.

SEQ ID NO: 134—Amino acid sequence of TcGPAT9 from T. cacao.

SEQ ID NO: 135—Amino acid sequence of TcGPAT10 from T. cacao.

SEQ ID NO: 136—Amino acid sequence of TcGPAT11 from T. cacao.

SEQ ID NO: 137—Amino acid sequence of TcGPAT12 from T. cacao.

SEQ ID NO: 138—Amino acid sequence of TcGPAT13 from T. cacao.

SEQ ID NO: 139—Amino acid sequence of TcPDAT1 from T. cacao.

SEQ ID NO: 140—Amino acid sequence of TcPDAT2 from T. cacao.

SEQ ID NO: 141—Amino acid sequence of TcPDAT4 from T. cacao.

SEQ ID NO: 142—Amino acid sequence of TcPDAT5 from T. cacao.

SEQ ID NO: 143—Amino acid sequence of TcPDAT6 from T. cacao.

SEQ ID NO: 144—Codon optimised open reading frame encoding TcDGAT1 from T. cacao.

SEQ ID NO: 145—Codon optimised open reading frame encoding TcDGAT2 from T. cacao.

SEQ ID NO: 146—Codon optimised open reading frame encoding TcDGAT3 from T. cacao.

SEQ ID NO: 147—Codon optimised open reading frame encoding TcDGAT4 from T. cacao.

SEQ ID NO: 148—Codon optimised open reading frame encoding TcDGAT5 from T. cacao.

SEQ ID NO: 149—Codon optimised open reading frame encoding TcDGAT6 from T. cacao.

SEQ ID NO: 150—Codon optimised open reading frame encoding TcDGAT7 from T. cacao.

SEQ ID NO: 151—Codon optimised open reading frame encoding TcDGAT8 from T. cacao.

SEQ ID NO: 152—Codon optimised open reading frame encoding TcDGAT9 from T. cacao.

SEQ ID NO: 153—Codon optimised open reading frame encoding TcDGAT10 from T. cacao.

SEQ ID NO: 154—Codon optimised open reading frame encoding TcDGAT11 from T. cacao.

SEQ ID NO: 155—Codon optimised open reading frame encoding TcGPAT1 from T. cacao.

SEQ ID NO: 156—Codon optimised open reading frame encoding TcGPAT2 from T. cacao.

SEQ ID NO: 157—Codon optimised open reading frame encoding TcGPAT3 from T. cacao.

SEQ ID NO: 158—Codon optimised open reading frame encoding TcGPAT4 from T. cacao.

SEQ ID NO: 159—Codon optimised open reading frame encoding TcGPAT5 from T. cacao.

SEQ ID NO: 160—Codon optimised open reading frame encoding TcGPAT6 from T. cacao.

SEQ ID NO: 161—Codon optimised open reading frame encoding TcGPAT7 from T. cacao.

SEQ ID NO: 162—Codon optimised open reading frame encoding TcGPAT8 from T. cacao.

SEQ ID NO: 163—Codon optimised open reading frame encoding TcGPAT9 from T. cacao.

SEQ ID NO: 164—Codon optimised open reading frame encoding TcGPAT10 from T. cacao.

SEQ ID NO: 165—Codon optimised open reading frame encoding TcGPAT11 from T. cacao.

SEQ ID NO: 166—Codon optimised open reading frame encoding TcGPAT12 from T. cacao.

SEQ ID NO: 167—Codon optimised open reading frame encoding TcGPAT13 from T. cacao.

SEQ ID NO: 168—Codon optimised open reading frame encoding TcPDAT1 from T. cacao.

SEQ ID NO: 169—Codon optimised open reading frame encoding TcPDAT2 from T. cacao.

SEQ ID NO: 170—Codon optimised open reading frame encoding TcPDAT4 from T. cacao.

SEQ ID NO: 171—Codon optimised open reading frame encoding TcPDAT5 from T. cacao.

SEQ ID NO: 172—Codon optimised open reading frame encoding TcPDAT6 from T. cacao.

SEQ ID NO: 173—Nucleotide sequence of the cloning vector pNIz0hyg[dga2] used for insertion of genes for transformation of Y. lipolytica cells. Nucleotides 416-423 and 6134-6141, NotI restriction enzyme sites; nucleotides 424-736, 5′ portion of Y. lipolytica zeta sequence; nucleotides 737-742 and 5733-5738, BsaI restriction enzyme sites; nucleotides 743-1742, 5′-upstream sequence from the Y. lipolytica DGA2 gene; nucleotides 1743-1755, SfiI restriction enzyme site; nucleotides 1784-1811 and 4699 to 4732, loxPsym recombination sites; nucleotides 1829-2245 and 3460-3858, pTEF promoters; nucleotides 2246-3271, protein coding region of hygromycin B phosphotransferase (HygR); nucleotides 3859-4581, protein coding region of fuGFP polypeptide; nucleotides 3272-3453 and 4582-4698, lip2 polyadenylation/transcription terminators; nucleotides 4733-5732, 3′-downstream sequence from the Y. lipolytica DGA2 gene; nucleotides 5739-6141, 3′ portion of Y. lipolytica zeta sequence. 8397 nt.

SEQ ID NO:174—Nucleotide sequence of the cloning vector pNIz0ura[fad2] used for insertion of genes for transformation of Y. lipolytica cells. Nucleotides 416-423 and 5737-5744, NotI restriction enzyme sites; nucleotides 424-736, 5′ portion of Y. lipolytica zeta sequence; nucleotides 737-742 and 5320-5325, BsaI restriction enzyme sites; nucleotides 743-1742, 5′-upstream sequence from the Y. lipolytica FAD2 gene; nucleotides 1743-1755, SfiI restriction enzyme site; nucleotides 1784-1817 and 4286 to 4319, loxPsym recombination sites; nucleotides 1818-3037, URA3 selectable marker gene (in reverse orientation) including nucleotides 1999-2859, complement of the protein coding region of URA3; nucleotides 3038-3445, pTEF promoter; nucleotides 3446-4168, protein coding region of fuGFP polypeptide; nucleotides 4169-4285, lip2 polyadenylation/transcription terminators; nucleotides 4320-5319, 3′-downstream sequence from the Y. lipolytica FAD2 gene; nucleotides 5326-5728, 3′ portion of Y. lipolytica zeta sequence. 7984 nt.

SEQ ID NO:175—Nucleotide sequence of the cloning vector pNIz0nat[lro1] used for insertion of genes for transformation of Y. lipolytica cells. Nucleotides 416-423 and 5679-5686, NotI restriction enzyme sites; nucleotides 424-736, 5′ portion of Y. lipolytica zeta sequence; nucleotides 737-742 and 5320-5325, BsaI restriction enzyme sites; nucleotides 743-1742, 5′-upstream sequence from the Y. lipolytica FAD2 gene; nucleotides 1743-1755, SfiI restriction enzyme site; nucleotides 1784-1817 and 4244 to 4277, loxPsym recombination sites; nucleotides 1818-3037, URA3 selectable marker gene (in reverse orientation) including nucleotides 1999-2859, complement of the protein coding region of URA3; nucleotides 1883-2246 and 3005-3403, pTEF promoter; nucleotides 3404-4126, protein coding region of fuGFP polypeptide; nucleotides 2817-2998 and 4127-4243, lip2 polyadenylation/transcription terminators; nucleotides 4278-5277, 3′-downstream sequence from the Y. lipolytica FAD2 gene; nucleotides 5284-5678, 3′ portion of Y. lipolytica zeta sequence. 7942 nt.

SEQ ID NO:176—Nucleotide sequence of the cloning vector pNIz0hyg[pox2] used for insertion of genes for transformation of Y. lipolytica cells. Nucleotides 416-423 and 6134-6141, NotI restriction enzyme sites; nucleotides 424-736, 5′ portion of Y. lipolytica zeta sequence; nucleotides 737-742 and 5733-5738, BsaI restriction enzyme sites; nucleotides 743-1742, 5′-upstream sequence from the Y. lipolytica POX2 gene; nucleotides 1743-1755, SfiI restriction enzyme site; nucleotides 1784-1811 and 4699-4732, loxPsym recombination sites; nucleotides 1882-2245 and 3460-3858, pTEF promoters; nucleotides 2246-3271, protein coding region of hygromycin B phosphotransferase (HygR; SEQ ID NO: 4); nucleotides 3859-4581, protein coding region of fuGFP polypeptide; nucleotides 3272-3453 and 4582-4698, lip2 polyadenylation/transcription terminators; nucleotides 4733-5732, 3′-downstream sequence from the Y. lipolytica POX2 gene; nucleotides 5739-6141, 3′ portion of Y. lipolytica zeta sequence. 8397 nt.

### DETAILED DESCRIPTION OF THE INVENTION

### Abbreviations

Acetyl-CoA and Malonyl-CoA: acetyl-coenzyme A and malonyl-coenzyme A;
ACCase: Acetyl-CoA carboxylase;
FAS: fatty acid synthase complex;
KAS II: ketoacyl-ACP synthase II (EC 2.3.1.41);
PAP: PA phosphorylase (EC 3.1.3.4);
G3P: glycerol-3-phosphate;
LPA: lysophosphatidic acid;
PA: phosphatidic acid;
MAG: monoacylglycerol
DAG: diacylglycerol;
TAG: triacylglycerol;
Acyl-CoA: acyl-coenzyme A;
PC: phosphatidylcholine;
GPAT: glycerol-3-phosphate acyltransferase;
LPAAT: lysophosphatidic acid acyltransferase (EC 2.3.1.51);
LPCAT: acyl-CoA:lysophosphatidylcholine acyltransferase; or synonyms 1-acylglycerophosphocholine O-acyltransferase; acyl-CoA:1-acyl-sn-glycero-3-phosphocholine O-acyltransferase (EC 2.3.1.23);
CPT: CDP-choline:diacylglycerol cholinephosphotransferase; or synonyms 1-alkyl-2-acetylglycerol cholinephosphotransferase; alkylacylglycerol cholinephosphotransferase; cholinephosphotransferase; phosphorylcholine-glyceride transferase (EC 2.7.8.2);
PDCT: phosphatidylcholine:diacylglycerol cholinephosphotransferase;
PLC: phospholipase C (EC 3.1.4.3);
PLD: Phospholipase D; choline phosphatase; lecithinase D; lipophosphodiesterase II (EC 3.1.4.4);
PDAT: phospholipid:diacylglycerol acyltransferase; or synonym phospholipid:1,2-diacyl-sn-glycerol O-acyltransferase (EC 2.3.1.158);
FAD2: fatty acid Δ12-desaturase;
FAD3, fatty acid A15-desaturase;
UDP-Gal: Uridine diphosphate galactose.

### General Techniques and Definitions

Unless specifically defined otherwise, all technical and scientific terms used herein shall be taken to have the same meaning as commonly understood by one of ordinary skill in the art (e.g., in cell culture, fermentation, molecular genetics, protein chemistry, non-meat food products and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, a “lipid” is any of a class of organic compounds that are or comprise fatty acids, which may be esterified or non-esterified, or their derivatives and are insoluble in water but soluble in organic solvents, for example in chloroform. As used herein, lipids include non-polar lipids such as triacylglycerols (TAG), diacylglycerols (DAG) and monoacylglycerols (MAG) as well as waxes, wax esters, sterol esters, and polar lipids such as free fatty acids (FFA), phospholipids, galactolipids, ceramides and other sphingolipids. Lipids are composed of mainly carbon (C) and hydrogen (H) with some oxygen (O) and may also contain phosphorus (P), nitrogen (N) and sulphur (S). As used herein, the term “extracted lipid” refers to a lipid composition which has been extracted from a microbial cell. The extracted lipid can be a relatively crude composition obtained by, for example, lysing the cells, or a more purified composition where most, if not all, of one or more or each of the water, nucleic acids, proteins and carbohydrates derived from the cells have been removed. Examples of purification methods are described below. In an embodiment, the extracted lipid comprises at least about 10%, at least about 20%, at least about 30%, at least about 40%, at least about 50%, at least about 60%, at least about 70%, at least about 80%, at least about 90%, or at least about 95% (w/w) lipid by weight of the composition. In embodiments, the extracted lipid comprises between about 10% and 95% lipid by weight, for example between about 10% and about 50%, or about 50% and 95%, lipid by weight. The lipid may be solid or liquid at room temperature (25° C.), or a mixture of the two; when liquid it is considered to be an oil, when solid it is considered to be a fat. In an embodiment, extracted lipid of the invention has not been blended with another lipid produced from another source, for example, animal lipid. Alternatively, the extracted lipid may be blended with a different lipid.

As used herein, the term “polar lipid” refers to amphipathic lipid molecules having a hydrophilic head group and a hydrophobic tail of one or more hydrocarbon chains, for example one or more fatty acyl groups, including phospholipids (e.g. phosphatidylcholine, phosphatidylethanolamine, phosphatidylinositol, phosphatidylserine, phosphatidylglycerol, diphosphatidylglycerols), galactolipids, cephalins, sphingolipids (sphingomyelins and glycosphingolipids), phosphatidic acid, cardiolipin and glycoglycerolipids. The hydrophilic head group is either a charged or uncharged polar group at physiological pH, giving these lipids the amphiphilic character. Polar lipids have the ability to ionize and/or form hydrogen bonds through their hydrophilic head group with water molecules which cause an increase in their aqueous solubility relative to non-polar lipids. Phospholipids are composed of the following major structural units: fatty acids, glycerol and phosphoric acid, and may contain amino alcohols. They are considered to function mostly in cells as structural lipids, playing important roles in the structure of the membranes of plants, microbes and animals. Because of their chemical structure, polar lipids exhibit a bipolar nature, exhibiting solubility or partial solubility in both polar and non-polar solvents.

The term “phospholipid”, as used herein, refers to an amphipathic molecule, having a hydrophilic head group and a hydrophobic tail of one (in the case of lysophosphatidic acid) or two hydrocarbon chains, that has a glycerol backbone esterified to a phosphate-containing “head” group and the fatty acid(s) which provide the hydrophobic tail. The phosphate group can be covalently linked with simple organic molecules such as choline, ethanolamine or serine. Due to their charged headgroup at neutral pH, phospholipids are polar lipids, having some solubility in solvents such as ethanol in addition to solvents such as chloroform. Phospholipids are a key component of all cell membranes. They can form lipid bilayers because of their amphiphilic characteristic. Well known phospholipids include phosphatidylcholine (PC), phosphatidylethanolamine (PE), phosphatidylinositol (PI), phosphatidylserine (PS), phosphatidic acid (PA), phosphatidylglycerol (PG) and cardiolipin.

As used herein, the term “non-polar lipid” refers to lipids that have a hydrophobic “tail” of one or more hydrocarbon groups, for example one or more acyl chains, but are not amphipathic molecules, lacking a hydrophilic head group that the polar lipids have. The fatty acids in non-polar lipids are in an esterified form; free (unesterified) fatty acids are considered herein as polar lipids. Examples of esterified forms include, but are not limited to, triacylglycerol (TAG), diacylyglycerol (DAG) and monoacylglycerol (MAG). Non-polar lipids also include sterol esters and wax esters. Non-polar lipids are also known as “neutral lipids” since they are essentially uncharged at physiological pH. Nonpolar lipids such as TAG and sterol esters do not form a monomolecular layer (monolayer) at an air-water or oil-water interface and so are essentially insoluble in aqueous solutions. In contrast, polar lipids can form a monolayer at an air-water or oil-water interface, and can form micelles or bilayers in an aqueous phase. Non-polar lipid may be a liquid at room temperature, or a solid, depending on the degree of unsaturation of the fatty acids in the non-polar lipid. Typically, the more saturated the fatty acid content, the higher the melting temperature of the lipid. Extracted lipids which are a liquid at room temperature are referred herein as “oils” whereas those which are solid at room temperature are referred to herein as “fats”.

As used herein, the term “fatty acid” refers to a carboxylic acid consisting of an aliphatic hydrocarbon chain and a terminal carboxyl group. The hydrocarbon chain can be either saturated or unsaturated. Unsaturated fatty acids include monounsaturated fatty acids having only one carbon-carbon double bond and polyunsaturated fatty acids (PUFA) having at least two carbon-carbon double bonds, typically between 2 and 6 carbon-carbon double bonds. A fatty acid may be a free fatty acid (FFA) or esterified to a glycerol or glycerol-phosphate molecule, CoA molecule or other headgroup as known in the art, preferably esterified as part of a polar lipid such as a phospholipid.

As used herein, the term “total fatty acid (TFA) content” or variations thereof refers to the total amount of fatty acids in, for example, the extracted lipid or cell, on a weight basis. In an example, the total fatty acid content includes a total saturated fatty acid content of saturated fatty acids (SFA) and a total monounsaturated fatty acid content of monounsaturated fatty acids (MUFA). The TFA may be expressed as a percentage of the weight of the cell or other fraction, e.g., as a percentage of the polar lipid. Unless otherwise specified, the weight with regard to the cell weight is the dry cell weight (DCW). In an embodiment, TFA content is measured by conversion of the fatty acids to fatty acid methyl esters (FAME) or fatty acid butyl esters (FABE) and measurement of the amount of FAME or FABE by GC, using addition of a known amount of a distinctive fatty acid standard as a quantitation standard in the GC. Typically, the amount and fatty acid composition of lipids comprising only fatty acids in the range of C10-C24 are determined by conversion to FAME, whereas lipids comprising fatty acids in the range of C4-C10 are determined by conversion to FABE. TFA therefore represents the weight of just the fatty acids, not the weight of the fatty acids and their linked moieties in the lipid.

“Saturated fatty acids” do not contain any double bonds or other functional groups along the acyl chain. The term “saturated” refers to hydrogen, in that all carbons (apart from the carboxylic acid [—COOH] group) contain as many hydrogens as possible. Examples of saturated fatty acids in lipid of the invention include stearic acid (C18:0), palmitic acid (C16:0), myristic acid (C14:0), arachidic acid (C20:0), behenic acid (C22:0) and lignoceric acid (C24:0).

“Unsaturated fatty acids” are of similar form to saturated fatty acids, except that one or more alkene functional groups exist along the chain, with each alkene substituting a singly-bonded “—CH₂—CH₂—” part of the chain with a doubly-bonded “—CH═CH—” portion (that is, a carbon double bonded to another carbon). The two next carbon atoms in the chain that are bound to either side of the double bond can occur in a cis or trans configuration, preferably in the cis configuration.

As used herein, the term “monounsaturated fatty acid” refers to a fatty acid which comprises at least 12 carbon atoms in its carbon chain and only one alkene group (carbon-carbon double bond) in the chain. Monounsaturated fatty acids include C12:1Δ9, C14:1Δ9, C16:1Δ9 (palmitoleic acid), C18:1Δ9 (oleic acid) and C18:1Δ11 (vaccenic acid).

As used herein, the terms “polyunsaturated fatty acid” or “PUFA” refer to a fatty acid which comprises at least 12 carbon atoms in its carbon chain and at least two alkene groups (carbon-carbon double bonds). Ordinarily, the number of carbon atoms in the carbon chain of the fatty acids refers to an unbranched carbon chain. Unless stated otherwise, if the carbon chain is branched, the number of carbon atoms excludes those in side groups. Polar lipids of the invention, such as in an extract or cell of the invention, comprise at least one ω6 fatty acid having a desaturation (carbon-carbon double bond) in the sixth carbon-carbon bond from the methyl end of the fatty acid. Examples of ω6 fatty acid include, but are not limited to, arachidonic acid (ARA, C20:4Δ5,8,11,14; ω6), dihomo-gammalinolenic acid (DGLA, C20:3Δ8,11,14; ω6), eicosadienoic acid (EDA, C20:2Δ11,14; ω6), docosatetraenoic acid (DTA, C22:4Δ7,10,13,16; ω6), docosapentaenoic acid-ω6 (DPA-ω6, C22:5Δ4,7,10,13,16; ω6), γ-linolenic acid (GLA, C18:3Δ6,9,12; ω6) and linoleic acid (LA, C18:2Δ9,12; ω6). In some embodiments, polar lipid of the invention, such as in an extract or cell of the invention, comprise at least one ω3 fatty acid having a desaturation (carbon-carbon double bond) in the third carbon-carbon bond from the methyl end of the fatty acid. In some embodiments, polar lipid of the invention, such as in an extract or cell of the invention, does not comprise specific ω3 fatty acids such as one or more of C16:3ω3, ALA, EPA and DHA, or does not comprise any ω3 fatty acids. Examples of ω3 fatty acids include, but are not limited to, α-linolenic acid (ALA, C18:3Δ9,12,15; ω3), hexadecatrienoic acid (C16:3ω3), eicosapentaenoic acid (EPA, C20:5Δ5,8,11,14,17; (3), docosapentaenoic acid (DPA, C22:5Δ7,10,13,16,19, ω3), docosahexaenoic acid (DHA, 22:6Δ4,7,10,13,16,19, (3), eicosatetraenoic acid (ETA, C20:4Δ8,11,14,17; (3) and eicosatrienoic acid (ETrA, C20:3Δ11,14,17; ω3). In some embodiments, polar lipid of the invention, such as in an extract or cell of the invention, does not comprise one or more or all of the following ω3 fatty acids; C16:3ω3, EPA and DHA.

As used herein, the term “L/S-SFA ratio” refers to the total amount of saturated fatty acids having 18 carbons or more (such as including stearic acid (C18:0), arachidic acid (C20:0), behenic acid (C22:0) and lignoceric acid (C24:0)) divided by the total amount of saturated fatty acids having 16 carbons or less (such as including palmitic acid (C16:0), myristic acid (C14:0) and larric acid (C:12:0).

As used herein, “C12:0” refers to lauric acid.

As used herein, “C14:0” refers to myristic acid.

As used herein, “C15:0” refers to n-pentadecanoic acid.

As used herein, “C16:0” refers to palmitic acid.

As used herein, “C17: Δ1” refers to heptadecenoic acid.

As used herein, “C16:1Δ9” refers to palmitoleic acid, or-hexadec-9-enoic acid.

As used herein, “C18:0” refers to stearic acid.

As used herein, “C18:1Δ9”, sometimes referred to in shorthand as “C18:1”, refers to oleic acid.

As used herein, “C18:1Δ11” refers to vaccenic acid.

As used herein, “C20:0” refers to eicosanoic acid which is also known as arachidic acid.

As used herein, “C20:1” refers to eicosenoic acid.

As used herein, “C22:0” refers to docosanoic acid which is also known as behenic acid.

As used herein, “C22:1” refers to erucic acid.

As used herein, “C24:0” refers to tetracosanoic acid which is also known as lignoceric acid.

“Triacylglyceride” or “TAG” is a glyceride in which the glycerol is esterified with three fatty acids which may be the same (e.g. as in tri-olein) or, more commonly, different. All three of the fatty acids may be different, or two of the fatty acids may be the same and the third is different. In the Kennedy pathway of TAG synthesis, DAG is formed as described below, and then a third acyl group is esterified to the glycerol backbone by the activity of a diglyceride acyltransferase (DGAT). TAG is a form of non-polar lipid. The three acyl groups esterified in a TAG molecule are referred to as being esterified in the sn-1, sn-2 and sn-3 positions, referring to the positions in the glycerol backbone of the TAG molecule. The sn-1 and sn-3 positions are chemically identical, but biochemically the acyl groups esterified in the sn-1 and sn-3 positions are distinct in that separate and distinct acyltransferase enzymes catalyse the esterifications.

“Diacylglyceride” or “DAG” is glyceride in which the glycerol is esterified with two fatty acids which may be the same or, preferably, different. As used herein, DAG comprises a hydroxyl group at a sn-1,3 or sn-2 position, and therefore DAG does not include phosphorylated glycerolipid molecules such as PA or PC. In the Kennedy pathway of DAG synthesis, the precursor sn-glycerol-3-phosphate (G3P) is esterified to two acyl groups, each coming from a fatty acid coenzyme A ester, in a first reaction catalysed by a glycerol-3-phosphate acyltransferase (GPAT) at position sn-1 to form LysoPA, followed by a second acylation at position sn-2 catalysed by a lysophosphatidic acid acyltransferase (LPAAT) to form phosphatidic acid (PA). This intermediate is then de-phosphorylated by PAP to form DAG.

As used herein, an “oil” is a composition comprising predominantly lipid and which is a liquid at room temperature.

As used herein, an “oleaginous” cell or microbe is one that is capable of storing at least 20% lipid, such as for example 20% to 70%, of its cell mass on a dry weight basis. The lipid content may depend on culture conditions, as is known in the art. It is understood that so long as the microbe is capable of synthesizing and accumulating at least 20% lipid on a dry cell weight basis under at least one set of culture conditions it is regarded as an oleaginous cell, even if under different conditions it accumulates less than 20% lipid. As used herein, a “microbe which is derived from an oleaginous microbe” is a microbe which is derived from a progenitor oleaginous microbe by one or more genetic modifications. The microbe which is derived from an oleaginous microbe may itself be an oleaginous microbe, or it may produce less than 20% lipid and not be an oleaginous microbe. The genetic modifications may have been introduced by human intervention or be naturally occurring, so long as at least one of the genetic modifications was introduced by human intervention. In an embodiment, the genetic modifications to produce the derived microbe comprise one or more genetic modifications which result in a reduced synthesis and/or accumulation of TAG.

As used herein, a “heterotrophic” cell is one that is capable of utilizing organic materials as a carbon source for metabolism and growth. Heterotrophic organisms may also be able to grow autotrophically under suitable conditions.

As used herein, “fermentation” refers to a metabolic process that produces chemical changes in organic molecules through the action of enzymes in the cells, under conditions either lacking oxygen or having reduced levels of oxygen relative to air.

As used herein, the term “volatile solvent” refers liquids that easily vaporize into a gas which can be used to disrupt and disperse microbial cells, and which may therefore be present in extracted lipid of the invention. Examples of volatile solvents which may be present in extracted lipid of the invention include, but are not limited, hexane and other alkanes, chloroform, ether, methanol, ethanol, propanol, and mixtures of any one or more thereof.

As used herein, the term “corresponding extracted microbial lipid obtained from a corresponding microbe lacking the at least one genetic modification” refers to lipid produced under the same culture conditions as the lipid produced from cells which have the at least one genetic modification.

### Fatty Acid Biosynthesis

The present invention relates to the genetic modification of microbial cells to increase saturated fatty acid production, particularly increase the production of saturated fatty acids which are 18 or more carbons in length. Details of genes which can be modified, either by expression of an exogenous polynucleotide, or modification of an endogenous gene are discussed below.

As used herein, the term “fatty acyl acyltransferase” refers to a protein which is capable of transferring an acyl group from an acyl-CoA, PC or acyl-ACP, preferably an acyl-CoA or PC, onto a substrate molecule to which the acyl group is transferred, linking the acyl group to the substrate molecule by a covalent bond, forming an ester linkage. Preferred substrate molecules are 3-phosphoglycerol, lysophosphatidic acid or diacylglycerol, to form MAG, DAG or TAG, respectively. These acyltransferases include DGAT, PDAT, MGAT, GPAT, LPAAT, and LPCAT.

### Diacylglycerol Acyltransferase (DGAT)

As used herein, the term “diacylglycerol acyltransferase” (DGAT), also known as acyl-CoA:diacylglycerol acyltransferase (EC 2.3.1.20), refers to a protein which transfers a fatty acyl group from an acyl-CoA to a DAG substrate to produce TAG. Thus, the term “diacylglycerol acyltransferase activity” refers to the transfer of an acyl group from acyl-CoA to DAG to produce TAG. A DGAT may also have monoacylglycerol acyltransferase (MGAT) activity but predominantly functions as a DGAT, i.e., it has greater catalytic activity as a DGAT than as a MGAT when the enzyme activity is expressed in units of nmoles product/min/mg protein (see for example, Yen et al., 2005). DGAT uses an acyl-CoA substrate as the acyl donor and transfers it to the sn-3 position of DAG to form TAG. The enzyme functions in its native state in the endoplasmic reticulum (ER) of the cell or, in the case of a soluble DGAT3, in the cytoplasm.

There are three known types of DGAT, referred to as DGAT1, DGAT2 and DGAT3. The diversity and relationships of the types of DGATs have been reviewed by Lung and Weselake (2006) and Turchetto-Zolet et al. (2011 and 2016). DGAT1 polypeptides are membrane proteins that often have 10 transmembrane domains although they may have only 6-9 transmembrane domains. They show sequence homology with sterol:acyl-CoA acyltransferase (ACAT; EC 2.3.1.26). Both enzymes belong to a large family of membrane-bound O-acyltransferases (MBOAT) proteins. DGAT2 polypeptides, initially identified in Mortierella rammanniana (Lardizabal et al., 2001), are also membrane proteins but have one or 2 transmembrane domains, whilst DGAT3 polypeptides typically have no transmembrane domains and are thought to be soluble in the cytoplasm. DGAT1 polypeptides from plant, animal and microbial sources typically have 510-550 amino acid residues while DGAT2 polypeptides from plants and animals typically have about 310-330 residues. DGAT2 is thought to be the main enzyme responsible for producing TAG from DAG in most microbial cells, for example in yeast Saccharomyces cerevisiae. The DGAT2 polypeptides are related in amino acid sequence to acyl-CoA:monoacylglycerol acyltransferases (MGAT, EC 2.3.1.22) and acyl-CoA wax-alcohol acyltransferase (AWAT, EC 2.3.1.75), Turchetto-Zolet et al. (2011).

Plant DGAT1 and DGAT2 appear to have non-redundant functions in triacylglycerol biosynthesis. DGAT1 is the main enzyme responsible for TAG synthesis during seed development. Examples of DGAT1 polypeptides include DGAT1 proteins from Yarrowia lipolytica, encoded by the YALI0 D07986 g gene (also known as the DGA2 gene, SEQ ID NO:55), Mortierella alpina (AQX34626.1), Aspergillus fumigatus (XP_755172.1), Arabidopsis thaliana (CAB44774.1), Ricinus communis (AAR11479.1), Vernicia fordii (ABC94472.1), Vernonia galamensis (ABV21945.1 and ABV21946.1), Euonymus alatus (AAV31083.1), Nannochloropsis oceanica (Zienkiewicz et al., 2017), Saccharomyces cerevisiae (Zulu et al., 2017), Caenorhabditis elegans (AAF82410.1), Rattus norvegicus (NP_445889.1), Homo sapiens (NP_036211.2), as well as variants and/or mutants thereof.

Examples of DGAT2 polypeptides include proteins encoded by DGAT2 genes from Arabidopsis thaliana (NP_566952.1), Ricinus communis (AAY16324.1), Vernicia fordii (ABC94474.1), Mortierella ramanniana (AAK84179.1), Homo sapiens (Q96PD7.2; Q58HT5.1), Bos taurus (Q70VZ8.1), Mus musculus (AAK84175.1), as well as variants and/or mutants thereof. DGAT1 and DGAT2 amino acid sequences show little homology in their amino acid sequences (Turchetto-Zolet et al., 2011, Turchetto-Zolet et al., 2016). For example, A. thaliana DGAT2 has a greater preference for the polyunsaturated linoleoyl-CoA and linolenoyl-CoA as acyl donors relative to the monounsaturated oleoyl-CoA, compared to DGAT1 from A. thaliana.

Examples of DGAT3 polypeptides include proteins encoded by DGAT3 genes from peanut (Arachis hypogaea, Saha, et al., 2006), as well as variants and/or mutants thereof. A DGAT has little or no detectable MGAT activity, for example, less than 300 pmol/min/mg protein, preferably less than 200 pmol/min/mg protein, more preferably less than 100 pmol/min/mg protein.

In an embodiment, an exogenous polynucleotide which encodes a DGAT comprises one or more of the following:
i) nucleotides encoding a polypeptide comprising amino acids whose sequence is set forth as any one of SEQ ID NOs: 53, 55 or 115 to 125,
ii) nucleotides encoding a polypeptide comprising an amino acid sequence which is at least 30% identical to any one or more of SEQ ID NOs: 53, 55 or 115 to 125,
iii) nucleotides having a sequence set forth as in any one of SEQ ID NOs: 52, 54 or 144 to 154,
iv) nucleotides having a sequence which is at least 30% identical to one or more of SEQ ID NOs: 52, 54 or 144 to 154, or
v) a polynucleotide which hybridizes to one any one or more of i) to iv) under stringent conditions.

In an embodiment, an exogenous polynucleotide of the invention which encodes a DGAT1 comprises one or more of the following:
i) nucleotides encoding a polypeptide comprising amino acids whose sequence is set forth as SEQ ID NO:55, or a biologically active fragment thereof, or a polypeptide whose amino acid sequence is at least 30% identical to SEQ ID NO:55,
ii) nucleotides whose sequence is at least 30% identical, at least 40% identical, or at least 95% identical to i), and
iii) a polynucleotide which hybridizes to one or both of i) or ii) under stringent conditions.

In an embodiment, an exogenous polynucleotide of the invention which encodes a DGAT2 comprises one or more of the following:
i) nucleotides encoding a polypeptide comprising amino acids whose sequence is set forth as SEQ ID NO:53, or a biologically active fragment thereof, or a polypeptide whose amino acid sequence is at least 30% identical, at least 40% identical, or at least 95% identical to SEQ ID NO:53,
ii) nucleotides whose sequence is at least 30% identical to i), and
iii) a polynucleotide which hybridizes to one or both of i) or ii) under stringent conditions.

In an embodiment, the DGAT does not comprises amino acids having a sequence as provided in SEQ ID NO: 115 or SEQ ID NO: 116.

The TcDGAT1 polypeptide (SEQ ID NO: 115) is of the DGAT1 class (PLN02401) and is predicted by software available from www.cbs.dtu.dk/services/TMHMM/ to have 9 transmembrane domains. It has an MBOAT domain (pfam03062) at amino acids 266-489. The TcDGAT2 polypeptide (SEQ ID NO: 116) is of the DGAT2 class (PLN02783) and is predicted by the software to have 2 transmembrane domains. It has a DAGAT domain (pfam03982) at amino acids 74-318. TcDGAT3-8 all have homology to wax ester synthases (WES, pfam03007), which are also acyltransferases. TcDGAT3 (SEQ ID NO: 117) is predicted to have no transmembrane domains but has a WES domain at amino acids 64-268. TcDGAT4 (SEQ ID NO: 118) is predicted to have one transmembrane domain and has a WES domain at amino acids 128-276. TcDGAT5 (SEQ ID NO: 119) is predicted to have one transmembrane domain and has a WES domain at amino acids 146-303. TcDGAT6 (SEQ ID NO: 120) is predicted to have one transmembrane domain and has a WES domain at amino acids 102-291. TcDGAT7 (SEQ ID NO: 121) is predicted to have no transmembrane domains and does not have an identified WES domain. TcDGAT8 (SEQ ID NO: 122) is predicted to have one transmembrane domain and has a WES condensation domain (cd19533) at amino acids 36-177. TcDGAT9-11 all have homology to yeast LRO1 which in the PDAT family (PLN02517). TcDGAT9 (SEQ ID NO: 123) is predicted to have one transmembrane domain and has a LCAT domain (pfam02450) at amino acids 134-630. TcDGAT10 (SEQ ID NO:124) is predicted to have no transmembrane domains but has a LCAT domain (pfam02450) at amino acids 148-651. TcDGAT11 (SEQ ID NO:125) is predicted to have one transmembrane domain and has a LCAT domain (pfam02450) at amino acids 150-645.

As used herein, a “DGA1 polypeptide” is a polypeptide which has at least 30% sequence identity along the full length of SEQ ID NO:53, which is the amino acid sequence of the Y. lipolytica DGA1 polypeptide. As used herein, a functional DGA1 polypeptide is a DGA1 polypeptide which is capable of forming TAG from DAG and an acyl-CoA. A large number of DGA1 polypeptides have been reported and sequences are available in databases, for example Accession No. NC_001147.6 (794076 . . . 95332, complement), Saccharomyces cerevisiae Chromosome XV, gene YOR245C; Accession No. KABA2_02S14982, Kazachstania barnettii; Accession No. NC_030983.1 Chromosome VIII (552596 . . . 553852, complement), Saccharomyces eubayanus; Accession No. NC_005784.3 Chromosome III (594117 . . . 595502, complement), Eremothecium gossypii etc. As used herein, a “Yarrowia DGA1 polypeptide” is a polypeptide which has at least 95% sequence identity along the full length of SEQ ID NO:53 and which has DGAT activity. Aside from SEQ ID NO:53, examples of Yarrowia DGA1 polypeptide sequences include Accession Nos: QNQ00885.1 (513/514 identical); KAG5365696.1 (496/514 identical) and KAG5357621.1 (494/514 identical).

As used herein, a “DGA2 polypeptide” is a polypeptide which has at least 30% sequence identity along the full length of SEQ ID NO:55, which is the amino acid sequence of the Y. lipolytica DGA2 polypeptide. As used herein, a functional DGA2 polypeptide is a DGA2 polypeptide which is capable of forming TAG from DAG and an acyl-CoA. A large number of DGA2 polypeptides have been reported and sequences are available in databases. As used herein, a “Yarrowia DGA2 polypeptide” is a polypeptide which has at least 95% sequence identity along the full length of SEQ ID NO:55 and which has DGAT activity. Aside from SEQ ID NO:55, examples of Yarrowia DGA2 polypeptide sequences include Accession Nos: RDW42020.1 (525/526 identical); KAG5361387.1 (495/526 identical) and KAG5358063.1 (494/526 identical).

### Phospholipid:Diacylglycerol Acyltransferase (PDAT)

As used herein, the term “phospholipid:diacylglycerol acyltransferase” (PDAT; EC 2.3.1.158) or its synonym “phospholipid:1,2-diacyl-sn-glycerol O-acyltransferase” means an acyltransferase that transfers an acyl group from a phospholipid, typically from the sn-2 position of PC, to the sn-3 position of DAG to form TAG and lysophosphocholine (LPC). This reaction is different to DGAT and uses phospholipids as the acyl-donors. Increased expression of PDAT such as PDAT1, which may be exogenous or endogenous to the cell of the invention, increases the production of TAG from PC. The enzyme LPCAT can re-acylate the LPC to form more PC, allowing for continued production of TAG by PDAT. There are several forms of PDAT in plant cells including PDAT1, PDAT2 or PDAT3 (Ghosal et al., 2007).

In an embodiment, an exogenous polynucleotide which encodes a PDAT comprises one or more of the following:
i) nucleotides encoding a polypeptide comprising amino acids whose sequence is set forth as any one of SEQ ID NOs: 57 or 139 to 143,
ii) nucleotides encoding a polypeptide comprising an amino acid sequence which is at least 30% identical to any one or more of SEQ ID NOs: 57 or 139 to 143,
iii) nucleotides having a sequence set forth as in any one of SEQ ID NOs: 56 or 168 to 172,
iv) nucleotides having a sequence which is at least 30% identical to one or more of SEQ ID NOs: 56 or 168 to 172, or
v) a polynucleotide which hybridizes to one any one or more of i) to iv) under stringent conditions.

However, any PDAT encoding gene can be used. Homologs and naturally occurring variants of PDATs from microbial or plant, fungal or algal species can readily be identified and used in the present invention. In an embodiment, the homolog or variant is at least 95% identical, preferably at least 99% identical, to the amino acid sequence of the listed SEQ ID NO or Accession No. The PDAT may be exogenous or endogenous to the microbe of the invention.

### Monoacylglycerol Acyltransferase (MGAT)

As used herein, the term “monoacylglycerol acyltransferase” or “MGAT” refers to a protein which transfers a fatty acyl group from acyl-CoA to a MAG substrate, for example sn-2 MAG, to produce DAG. Thus, the term “monoacylglycerol acyltransferase activity” at least refers to the transfer of an acyl group from acyl-CoA to MAG to produce DAG. The term “MGAT” as used herein includes enzymes that act on sn-1/3 MAG and/or sn-2 MAG substrates to form sn-1,3 DAG and/or sn-1,2/2,3-DAG, respectively. In a preferred embodiment, the MGAT has a preference for sn-2 MAG substrate relative to sn-1 MAG, or substantially uses only sn-2 MAG as substrate. As used herein, MGAT does not include enzymes which transfer an acyl group preferentially to LysoPA relative to MAG, such enzymes are known as LPAATs. That is, a MGAT preferentially uses non-phosphorylated monoacyl substrates, even though they may also have low catalytic activity on LysoPA. A preferred MGAT does not have detectable activity in acylating LysoPA. A MGAT may also have DGAT function but predominantly functions as a MGAT, i.e., it has greater catalytic activity as a MGAT than as a DGAT when the enzyme activity is expressed in units of nmoles product/min/mg protein (also see Yen et al., 2002). There are three known classes of MGAT, referred to as, MGAT1, MGAT2 and MGAT3, respectively. Examples of MGAT1, MGAT2 and MGAT3 polypeptides are described in WO2013/096993.

sn-glycerol-3-phosphate acyltransferase (GPAT)

One important component in glycerolipid synthesis from fatty acids esterified to ACP or CoA is the enzyme sn-glycerol-3-phosphate acyltransferase (GPAT), which is another of the polypeptides involved in the biosynthesis of non-polar lipids. This enzyme catalyses the following reaction: G3P+fatty acyl-ACP or -CoA→LPA+free-ACP or -CoA. There are at least three different types of GPAT enzymes, a soluble form localized in plastidial stroma which uses acyl-ACP as its natural acyl substrate, and two membrane-bound forms localized in the ER and mitochondria which use acyl-CoA and acyl-ACP as natural acyl donors, respectively (Chen et al., 2011).

As used herein, the term “glycerol-3-phosphate acyltransferase” (GPAT; EC 2.3.1.15) and its synonym “glycerol-3-phosphate O-acyltransferase” refer to a protein which acylates glycerol-3-phosphate (G-3-P) to form LysoPA and/or MAG, the latter product forming if the GPAT also has phosphatase activity on LysoPA. The acyl group that is transferred is from acyl-CoA if the GPAT is an ER-type GPAT (an “acyl-CoA:sn-glycerol-3-phosphate 1-O-acyltransferase” also referred to as “microsomal GPAT”) or from acyl-ACP if the GPAT is a plastidial-type GPAT. Thus, the term “glycerol-3-phosphate acyltransferase activity” refers to the acylation of G-3-P to form LysoPA and/or MAG. The term “GPAT” encompasses enzymes that acylate G-3-P to form sn-1 LPA and/or sn-2 LPA. Preferably, the GPAT which may be over-expressed in the cell is a membrane bound GPAT that functions in the ER of the cell, more preferably a GPAT9.

The GPAT family is large and all known members contain two conserved domains, a plsC acyltransferase domain (PF01553) and a HAD-like hydrolase (PF12710) superfamily domain and variants thereof. In addition to this, at least in Arabidopsis thaliana, GPATs in the subclasses GPAT4-GPAT8 all contain a N-terminal region homologous to a phosphoserine phosphatase domain (PF00702), and GPATs which produce MAG as a product can be identified by the presence of such a homologous region.

Homologues of Arabidopsis GPAT4 (Accession No. NP_171667.1) and GPAT6 (NP_181346.1) include AAF02784.1 (Arabidopsis thaliana), AAL32544.1 (Arabidopsis thaliana), AAP03413.1 (Oryza sativa), ABK25381.1 (Picea sitchensis), ACN34546.1 (Zea Mays), BAF00762.1 (Arabidopsis thaliana), BAH00933.1 (Oryza sativa), EAY84189.1 (Oryza sativa), EAY98245.1 (Oryza sativa), EAZ21484.1 (Oryza sativa), EEC71826.1 (Oryza sativa), EEC76137.1 (Oryza sativa), EEE59882.1 (Oryza sativa), EFJ08963.1 (Selaginella moellendorffii), EFJ11200.1 (Selaginella moellendorffii), NP_001044839.1 (Oryza sativa), NP_001045668.1 (Oryza sativa), NP_001147442.1 (Zea mays), NP_001149307.1 (Zea mays), NP_001168351.1 (Zea mays), AFH02724.1 (Brassica napus) NP_191950.2 (Arabidopsis thaliana), XP_001765001.1 (Physcomitrella patens), XP_001769671.1 (Physcomitrella patens), (Vitis vinifera), XP_002275348.1 (Vitis vinifera), XP_002276032.1 (Vitis vinifera), XP_002279091.1 (Vitis vinifera), XP_002309124.1 (Populus trichocarpa), XP_002309276.1 (Populus trichocarpa), XP_002322752.1 (Populus trichocarpa), XP_002323563.1 (Populus trichocarpa), XP_002439887.1 (Sorghum bicolor), XP_002458786.1 (Sorghum bicolor), XP_002463916.1 (Sorghum bicolor), XP_002464630.1 (Sorghum bicolor), XP_002511873.1 (Ricinus communis), XP_002517438.1 (Ricinus communis), XP_002520171.1 (Ricinus communis), ACT32032.1 (Vernicia fordii), NP_001051189.1 (Oryza sativa), AFH02725.1 (Brassica napus), XP_002320138.1 (Populus trichocarpa), XP_002451377.1 (Sorghum bicolor), XP_002531350.1 (Ricinus communis), and XP_002889361.1 (Arabidopsis lyrata).

The soluble plastidial GPATs have been purified and genes encoding them cloned from several plant species such as pea (Pisum sativum, Accession number: P30706.1), spinach (Spinacia oleracea, Accession number: Q43869.1), squash (Cucurbita moschate, Accession number: P10349.1), cucumber (Cucumis sativus, Accession number: Q39639.1) and Arabidopsis thaliana (Accession number: Q43307.2).

In an embodiment, an exogenous polynucleotide which encodes a GPAT comprises one or more of the following:
i) nucleotides encoding a polypeptide comprising amino acids having a sequence as set forth in any one of SEQ ID NOs: 126 to 138,
ii) nucleotides encoding a polypeptide comprising an amino acid sequence which is at least 30% identical to any one or more of SEQ ID NO's 126 to 138,
iii) nucleotides having a sequence set forth as in any one of SEQ ID NOs: 155 to 167, iv) nucleotides having a sequence which is at least 30% identical to one or more of SEQ ID NOs: 155 to 167, or
v) a polynucleotide which hybridizes to one any one or more of i) to iv) under stringent conditions.
   1-Acyl-sn-glycerophosphate acyltransferase (LPAAT)

As used herein, the term “lysophosphatidic acid acyltransferase” (LPAAT; EC 2.3.1.51) and its synonyms “1-acyl-glycerol-3-phosphate acyltransferase”, “acyl-CoA:1-acyl-sn-glycerol-3-phosphate 2-O-acyltransferase” and “1-acylglycerol-3-phosphate O-acyltransferase” refer to a protein which acylates lysophosphatidic acid (LPA) to form phosphatidic acid (PA). The acyl group that is transferred is from acyl-CoA if the LPAAT is an ER-type LPAAT or from acyl-ACP if the LPAAT is a plastidial-type LPAAT. Thus, the term “lysophosphatidic acid acyltransferase activity” refers to the acylation of LPA to form PA.

acyl-ACP thioesterase

As used herein, the term “acyl-ACP thioesterase” refers to an enzyme that hydrolyses a thioester bond in an acyl-acyl carrier protein (ACP) substrate (EC 3.1.2.14). They are plastid-targeted soluble enzymes encoded by nuclear genes in plants and other eukaryotes. Acyl-ACP thioesterases cleave acyl groups from ACP and thereby terminate the acyl chain elongation activity in fatty acid synthesis by fatty acid synthase (FAS) in the plastids where fatty acid synthesis occurs. This terminal step releases free fatty acids within the plastids followed by their conversion to acyl-CoA thioesters for export from the plastid to the cytoplasm and endoplasmic reticulum for the formation of TAG, other neutral lipids and phospholipids. Fatty acyl-ACP thioesterases are classified into two distinct but closely related classes on the basis of amino acid alignments (Martins-Noguerol et al. 2020) and activity, namely FATB acyl-ACP thioesterases which have specificity mainly for C16 and shorter SFA, and FATA acyl-ACP thioesterases which are more active on C18-ACP than C16-ACP substrates, particularly more active on MUFA-ACP substrates such as oleoyl-ACP, but may also having activity on stearoyl-ACP (Jones et al., 1995; Salas and Ohlrogge, 2002). Hawkins and Kridl (1998) reported that enzymes from both the FATA and FATB classes have limited activity on stearoyl-ACP, but none of them prefer that substrate relative to oleoyl-ACP. However, more recent reports (Ghosh et al., 2007) have identified a FATB thioesterase from mahua (Madhuca latifolia, Accession No. AAX51637) and from Jatropha curcas (Dani et al., 2011; Accession No. ACT09366) with specificity for stearoyl-ACP over oleoyl- and palmitoyl-ACP. As used herein, a “FATA polypeptide” or “FATA thioesterase” refers to an acyl-ACP thioesterase of the FATA class, and a “FATB polypeptide” or “FATB thioesterase” refers to an acyl-ACP thioesterase of the FATB class.

Over-expression of FATA polypeptides in transgenic plants in some cases increases stearate levels in the seedoil, but in other cases does not (Hawkins and Kridl, 1998).

Bhattacharjee et al., (2011) identified a FATA thioesterase from mango, Mangifera indica, which had a relative substrate specificity of 100:35:1.8 towards oleoyl-, stearoyl- and palmitoyl-ACP, respectively. As described herein, the inventors selected two related FATA acyl-ACP thioesterases from the tropical plant mangosteen, Garcinia mangostana, designated GarmFATA1 (Accession No. U92876) and GarmFATA2 (Hawkins and Kridl, 1998). Both proteins have an N-terminal transit peptide sequence (TPS) which function to direct the proteins into plastids. The former polypeptide including its TPS has 352 amino acids whereas the latter including its TPS has 355 amino acids. The amino acid sequences of GarmFATA1 and GarmFATA2 are 73% identical along the full length of GarmFATA1. GarmFATA1 had its greatest activity on C18:1-ACP, with about 7-fold less activity on C18:0-ACP and less again on C16:0-ACP, exhibiting relative activities of 100:15:6 on oleoyl-, stearoyl- and palmitoyl-ACP, respectively. In contrast, GarmFATA2 had about 50-fold less activity on C18:0-ACP relative to C18:1-ACP. Both enzymes therefore had their main activity for the MUFA, C18:1-ACP. The amino acid sequences of GarmFATA1 and GarmFATA2 are provided herein as SEQ ID NO:83 and SEQ ID NO:85.

### Desaturases

As used herein, the term “desaturase” refers to an enzyme which is capable of introducing a carbon-carbon double bond into the acyl group of a fatty acid substrate which is typically in an esterified form such as, for example, acyl-CoA esters. The acyl group may be esterified to a phospholipid such as phosphatidylcholine (PC), or to acyl carrier protein (ACP), or preferably to CoA. Desaturases generally may be categorized into three groups accordingly. In one embodiment, the desaturase is a front-end desaturase.

As used herein, the term “front-end desaturase” refers to a member of a class of enzymes that introduce a double bond between the carboxyl group and a pre-existing unsaturated part of the acyl chain of lipids, which are characterized structurally by the presence of an N-terminal cytochrome b5 domain, along with a typical fatty acid desaturase domain that includes three highly conserved histidine boxes (Napier et al., 1997).

As used herein, a “Δ12-desaturase” refers to a protein which is capable of performing a desaturase reaction that introduces a carbon-carbon double bond at the 12^{th} carbon-carbon bond from the carboxyl end of a fatty acid substrate. Δ12-desaturases typically convert either oleoyl-phosphatidylcholine or oleoyl-CoA to linoleoyl-phosphatidylcholine (C18:1-PC) or linoleoyl-CoA (C18:1-CoA), respectively. The subclass using the PC linked substrate are referred to as phospholipid-dependent Δ12-desaturases, the latter subclass as acyl-CoA dependent Δ12-desaturases. Plant and fungal Δ12-desaturases are generally of the former subclass, whereas animal Δ12-desaturases, with the exception of some lower animal Δ12-desaturases such as C. elegans Δ12-desaturase, are generally of the latter subclass, for example the Δ12-desaturases encoded by genes cloned from insects by Zhou et al. (2008). Many other Δ12-desaturase sequences can be easily identified by searching sequence databases. Genes encoding numerous desaturases have been isolated from fungal sources. U.S. Pat. No. 7,211,656 describes a Δ12 desaturase from Saprolegnia diclina. WO2009016202 describes fungal desaturases from Helobdella robusta, Laccaria bicolor, Lottia gigantea, Microcoleus chthonoplastes, Monosiga brevicollis, Mycosphaerella fjiensis, Mycospaerella graminicola, Naegleria gruben, Nectria haematococca, Nematostella vectensis, Phycomyces blakesleeanus, Trichoderma resii, Physcomitrella patens, Postia placenta, Selaginella moellendorffih and Microdochium nivale. WO2005/012316 describes a Δ12-desaturase from Thalassiosira pseudonana and other fungi. WO2003/099216 describes genes encoding fungal Δ12-desaturases isolated from Neurospora crassa, Aspergillus nidulans, Botrytis cinerea and Mortierella alpina.

### Other Genes

In addition to the manipulation of the expression of the above enzymes, production of saturated fatty acids in lipid of microbial cells can be enhanced by genetic modification to modulate expression of one or more endogenous genes involved in microbial fatty acid biosynthesis, catabolism and regulation. Such exemplary microbial genes are provided in Table 1.

In some embodiments, the genetic modification(s) that increase the production of saturated fatty acids in lipid provide for increased expression and/or activity of one or more genes in Table 1. In some embodiments, the genetic modification(s) provide for increased expression and/or activity of a fatty acid synthesis gene (see Table 1 for examples). In some embodiments, the genetic modification(s) provide for increased expression and/or activity of a phospholipid synthesis gene (see Table 1 for examples). In some embodiments, the genetic modification(s) provide for increased expression and/or activity of a lipid synthesis regulating gene (see Table 1 for examples).

TABLE 1
Microbial genes and the accession numbers of encoded proteins involved
in fatty acid and lipid synthesis, catabolismand regulation.

| | Enzyme | | Gene in | Gene in | Gene in |
|---|---|---|---|---|---|
| Enzyme or protein | Commission No. | Gene | S. cerevisiae | Y. lipolytica | Pichia pastoris |
| Fatty acid synthesis | | | | | |
| and TAG production | | | | | |
| Acetyl-CoA synthetase | EC 6.2.1.1 | ACS1 | YAL054C | YALI0F05962g | ANZ76230.1 |
| Acetyl-CoA synthetase | EC 6.2.1.1 | ACS2 | YLR153C | none | ANZ73211.1 |
| ATP-citrate lyase | EC 2.3.3.8 | ACL1 | none | YALI0E34793g | ANZ75267.1 |
| subunit | | | | | |
| ATP-citrate lyase | EC 2.3.3.8 | ACL2 | none | YALI0D24431g | none |
| subunit | | | | | |
| Acetyl-CoA | EC 6.3.4.14 & | ACC1 | FAS3 | YALI0C11407g | ANZ73439.1 |
| carboxylase | EC 6.4.1.2 | | (accession no. | | |
| | | | M92156) | | |
| | | | YNR016C | | |
| Fatty acid synthase | EC 2.3.1.86 | FAS1 | YKL182W | YALI0B15059g | ANZ74209.1 |
| subunit beta | | | | | |
| Fatty acid synthase | EC 2.3.1.86 | FAS2 | YPL231W | YALI0B19382g | ANZ73614.1 |
| subunit alpha | | | | | |
| Fatty acid elongase | EC 2.3.1.199 | ELO1 | YJL196C | YALI0F06754g | ANZ76899.1 |
| Fatty acid elongase | EC 2.3.1.199 | ELO2 | YCR034W | YALI0B20196g | ANZ74046.1 |
| Fatty acid elongase | EC 2.3.1.199 | ELO3 | YLR372W | none | ANZ73325.1 |
| Δ5 Fatty acid | EC 1.14.19.30 | DES1 | none | none | none |
| desaturase | | | | | |
| Δ9 Fatty acid | EC 1.14.19.1 | OLE1 or | YGL055W | YALI0C05951g | ANZ77426.1 |
| desaturase | | SCD | | | |
| Δ12 Fatty acid | EC 1.14.19.6 | FAD2 | none | YALI0B10153g | AAX20125.1 |
| desaturase | | | | | |
| Δ15/ω3 Fatty acid | EC:1.14.19.13 | FAD3 | none | none | ABL63813.1 |
| desaturase | | | | | |
| Glycerol kinase | EC 2.7.1.30 | GUT1 | YHL032C | YALI0F00484g | ANZ77337.1 |
| G3P dehydrogenase | EC 1.1.1.18 | GPD1 | YDL022W | YALI0B02948g | ANZ75813.1 |
| (NAD) | | | | | |
| G3P dehydrogenase | EC 1.1.1.18 | GPD2 | YOL059W | none | none |
| (NAD) | | | | | |
| G3P dehydrogenase | EC 1.1.99.5 | GUT2 | YIL155C | YALI0B13970g | ANZ73466.1 |
| (mitochondria) | | | | | |
| Glycerophosphate | EC 2.3.1.15 | SCT1 | YBL011W | YALI0C00209g | ANZ74560.1 |
| acyltransferase (GPAT) | | | | | |
| Glycerophosphate | EC 2.3.1.15 & | GPT2 | YKR067W | none | ANZ73642.1 |
| acyltransferase (GPAT) | EC 2.3.1.42 | | | | |
| 1-Acyl-sn- | EC 2.3.1.51 | SLC1 | YDL052C | YALI0E18964g | ANZ73792.1 |
| glycerophosphate | | | | | |
| acyltransferase | | | | | |
| (LPAAT) | | | | | |
| 1-Acyl-sn- | EC 2.3.1.51 | ALE1 | YOR175C | YALI0F19514g | ANZ74296.1 |
| glycerophosphate | | | | | |
| acyltransferase | | | | | |
| (LPAAT) | | | | | |
| 1-Acyl-sn- | EC 2.3.1.51 | LOA | YPR139C | YALI0C14014g | ANZ76093.1 |
| glycerophosphate | | | | | |
| acyltransferase | | | | | |
| (LPAAT) | | | | | |
| Phosphatidic acid | EC 3.1.3.4 | PAH1 | YMR165C | YALI0D27016g | ANZ74484.1 |
| phosphatase (PAP) - | | | | | |
| phosphohydrolase | | | | | |
| Phosphatidic acid | EC 3.1.3.4 | APP1 | YNL094W | YALI0D02233g | ANZ75274.1 |
| phosphatase (PAP) | | | | | |
| Diacylglycerol | EC 2.3.1.20 | DGA1 | YOR245C | YALI0E32769g | ANZ74314.1 |
| acyltransferase | | | | | |
| (DGAT) | | | | | |
| Diacylglycerol | EC 2.3.1.20 | DGA2 | none | YALI0D07986g | none |
| acyltransferase | | | | | |
| (DGAT) | | | | | |
| Phospholipid:diacylglycerol | EC 2.3.1.158 | LRO1 | YNR008W | YALI0E16797g | ANZ75160.1 |
| acyltransferase (PDAT) | | | | | |
| Acyl-CoA:sterol | EC 2.3.1.26 | ARE1 | YCR048W | YALI0F06578g | ANZ73460.1 |
| acyltransferase | | | | | |
| Acyl-CoA:sterol | EC 2.3.1.26 | ARE2 | YNR019W | none | none |
| acyltransferase | | | | | |
| Cardiolipin synthase | EC 2.7.8.5 | PGS1 | YCL004W | YALI0F23837g | ANZ73566.1 |
| Fatty acyl-CoA | EC 6.2.1.3 | FAA1 | YOR317W | YALI0D17864g | ANZ75849.1 |
| synthetase | | | | | |
| Enzymes involved in | | | | | |
| PL synthesis | | | | | |
| Inositol-3-phosphate | EC 5.5.1.4 | INO1 | YJL153C | YALI0B04312g | AAC33791.1 |
| synthase | | | | | |
| Phosphatidate | EC 2.7.7.41 | CDS1 | YBR029C | YALI0E14443g | ANZ74906.1 |
| cytidylyltransferase | | | | | |
| CDP-DAG synthase | EC 2.7.7.41 | TAM41 | YGR046W | YALI0C12276g | ANZ73236.1 |
| Phosphatidylinositol | EC 2.7.8.11 | PIS1 | YPR113W | YALI0F20328g | ANZ76102.1 |
| synthase | | | | | |
| Phosphatidylserine | EC 2.7.8.8 | CHO1 | YER026C | YALI0D08514g | ANZ74203.1 |
| synthase | | | | | |
| Phosphatidylserine | EC 4.1.1.65 | PSD1 | YNL169C | YALI0D21604g | ANZ73933.1 |
| decarboxylase | | | | | |
| Phosphatidylserine | EC 4.1.1.65 | PSD2 | YGR170W | YALI0D03480g | ANZ76834.1 |
| decarboxylase | | | | | |
| Phosphatidylethanolamine | EC 2.1.1.17 | CHO2 | YGR157W | YALI0E06061g | ANZ73427.1 |
| methyltransferase | | | | | |
| Phospholipid | EC 2.1.1.17 & | OPI3 | YJR073C | YALI0E12441g | ANZ76546.1 |
| methyltransferase | EC 2.1.1.71 | | | | |
| Phosphatidylinositol | | PDR16 | YNL231C | YALI0A08448g | ANZ78024.1 |
| transfer protein | | | | | |
| Phosphatidylinositol/ | | CSR1 | YLR380W | YALI0C17545g | ANZ76329.1 |
| phosphatidylcholine | | | | | |
| transfer protein | | | | | |
| Phosphatidylinositol | EC 3.1.3.36 | SAC1 | YKL212W | YALI0D05995g | ANZ76554.1 |
| phosphatase | | | | | |
| Diacylglycerol kinase | EC 2.7.1.174 | DGK1 | YOR311C | YALI0F19052g | ANZ74498.1 |
| Enzymes involved in | | | | | |
| lipid catabolism | | | | | |
| Cholesterol | EC 3.1.1.13 | TGL1 | YKL140W | YALI0E32035g | ANZ73240.1 |
| esterase/TAG lipase | | | | | ANZ74310.1 |
| TAG lipase | EC 3.1.1.3 | TGL3 | YMR313C | YALI0D17534g | ANZ77507.1 |
| TAG lipase | EC 3.1.1.3 | TGL4 | YKR089C | YALI0F10010g | none |
| TAG lipase | EC 3.1.1.3 | TGL5 | YOR081C | none | ANZ75256.1 |
| Phospholipase B | EC 3.1.1.5 | PLB2 | YMR006C | none | ANZ75819.1 |
| Phospholipase B | EC 3.1.1.5 | PLB1 | YMR008C | YALI0E16060g | ANZ75299.1 |
| Phospholipase D | EC 3.1.4.4 | SPO14 | YKR031C | YALI0E18898g | ANZ76336.1 |
| Peroxisome biogenesis | | PEX10 | YDR265W | YALI0C01023g | ANZ77203.1 |
| factor 10 | | | | | |
| Acyl-CoA oxidase | EC 1.3.3.6 | POX1 | YGL205W | YALI0E32835g | ANZ76334.1 |
| Acyl-CoA oxidase | EC 1.3.3.6 | POX2 | YKR009C | YALI0F10857g | none |
| Acyl-CoA oxidase | EC 1.3.3.6 | POX3 | YIL160C | YALI0D24750g | none |
| Acyl-CoA oxidase | EC 1.3.3.6 | POX4 | none | YALI0E27654g | none |
| Acyl-CoA oxidase | EC 1.3.3.6 | POX5 | none | YALI0C23859g | none |
| Acyl-CoA oxidase | EC 1.3.3.6 | POX6 | none | YALI0E06567g | none |
| Protein kinase | EC 2.7.11.1 | SNF1 | YDR477W | YALI0D02101g | ANZ75125.1 |
| Multifunctional- | EC 4.2.1.74 | MFE1 | YKR009C | YALI0E15378g | ANZ74935.1 |
| oxidation protein | | | | | |
| Peroxisomal oxoacyl | EC 2.3.1.16 | POT1 | YIL160C | YALI0E18568g | ANZ75015.1 |
| thiolase | | | | | |
| Regulators of lipid | | | | | |
| synthesis | | | | | |
| Associated with Sit4 | EC 3.1.3.16 | SAP190 | YKR028W | YALI0F11869g | ANZ77566.1 |
| protein phosphatase | | | | | |
| Phosphoinositide 3- | EC 2.7.1.67 & | TOR2 | YKL203C | YALI0F07084g | ANZ75729.1 |
| kinase-related protein | EC 2.7.11.1 | | | | |
| kinase | | | | | |
| Phospholipid synthesis | | OPI1 | YHL020C | YALI0C14784g | ANZ73581.1 |
| regulatory protein | | | | | |

| | Gene in | Gene in | |
|---|---|---|---|
| Enzyme or protein | Mortierella alpina | E. coli | References |
| Fatty acid synthesis | | | |
| and TAG production | | | |
| Acetyl-CoA synthetase | KAF9286715.1 | P27550 | DeVirgilio, 1992 |
| Acetyl-CoA synthetase | none | none | Hiesinger et al., |
| | | | 1997 |
| ATP-citrate lyase | KAF9948114.1 | P75726 | Dulermo, 2015; |
| subunit | | | Feng et al., 2015 |
| ATP-citrate lyase | KAF9929232.1 | P0A9I1 | Dulermo, 2015; |
| subunit | | | Feng et al., 2015 |
| Acetyl-CoA | KAF9288230.1 | P0ABD5, | Al-Feel et al., 1992; |
| carboxylase | | P0ABD8, | Feng et al., 2015 |
| | | P24182, | |
| | | P0A9Q5 | |
| Fatty acid synthase | KAF9285532.1 | P0AAI9, | Janßen et al., 2014 |
| subunit beta | | P0A6R0, | |
| | | P0AEK2, | |
| | | P0A6Q3, | |
| | | P0A6Q6, | |
| | | P0AEK4, | |
| | | P0A953, | |
| | | P0AAI5 | |
| Fatty acid synthase | KAF9936935.1 | none | Schüller et al., 1992 |
| subunit alpha | | | |
| Fatty acid elongase | BAI40363.1 | none | Schneiter et al., |
| | | | 2000 |
| Fatty acid elongase | BAH02594.1 | none | Oh et al., 1997 |
| Fatty acid elongase | ADE06662.1 | none | Oh et al., 1997 |
| Δ5 Fatty acid | AAC39508.1 | none | Michaelson. 1998 |
| desaturase | | | |
| Δ9 Fatty acid | CAB38178.1 | MHO05803.1 | Stukey et al., 1989; |
| desaturase | | | Wongwathanarat et |
| | | | al., 1999 |
| Δ12 Fatty acid | Q9Y8H5.2 | none | Kassab et al., 2019 |
| desaturase | | | |
| Δ15/ω3 Fatty acid | Q59J82.1 | none | Kassab et al., 2019 |
| desaturase | | | |
| Glycerol kinase | KAF9275064.1 | P0A6F3 | Beopoulos et al., |
| | | | 2008 |
| G3P dehydrogenase | ALM55659.1 | P13035 | Beopoulos et al., |
| (NAD) | | | 2008 |
| G3P dehydrogenase | none | none | Beopoulos et al., |
| (NAD) | | | 2008 |
| G3P dehydrogenase | KAF9947304.1 | P0A9C0, | Beopoulos et al., |
| (mitochondria) | | P13033, | 2008 |
| | | P0A996 | |
| Glycerophosphate | KAF9941189.1 | P0A7A7 | Beopoulos et al., |
| acyltransferase (GPAT) | | | 2008; Janßen et al., |
| | | | 2014 |
| Glycerophosphate | KAF9964849.1 | P60782 | Beopoulos et al., |
| acyltransferase (GPAT) | | | 2008; Janßen et al., |
| | | | 2014 |
| 1-Acyl-sn- | AED33305 | P26647 | Beopoulos et al., |
| glycerophosphate | | | 2008; Janßen et al., |
| acyltransferase | | | 2014 |
| (LPAAT) | | | |
| 1-Acyl-sn- | KAF9279687.1 | none | Janßen et al., 2014 |
| glycerophosphate | | | |
| acyltransferase | | | |
| (LPAAT) | | | |
| 1-Acyl-sn- | KAF9967416.1 | none | Janßen et al., 2014 |
| glycerophosphate | | | |
| acyltransferase | | | |
| (LPAAT) | | | |
| Phosphatidic acid | none | none | Adeyo et al., 2011 |
| phosphatase (PAP) - | | | |
| phosphohydrolase | | | |
| Phosphatidic acid | KAF9924983.1 | P0A924 | Pascual et al., 2013 |
| phosphatase (PAP) | | | |
| Diacylglycerol | ATQ62217.1 | none | Beopoulos et al., |
| acyltransferase | | | 2008; Janßen et al., |
| (DGAT) | | | 2014 |
| Diacylglycerol | AQX34626.1 | none | Janßen et al., 2014 |
| acyltransferase | | | |
| (DGAT) | | | |
| Phospholipid:diacylgly | KAF9951579.1 | none | Beopoulos et al., |
| cerol acyltransferase | | | 2008 |
| (PDAT) | | | |
| Acyl-CoA:sterol | KAF9276817.1 | none | Beopoulos et al., |
| acyltransferase | | | 2008 |
| Acyl-CoA:sterol | none | none | Beopoulos et al., |
| acyltransferase | | | 2008 |
| Cardiolipin synthase | KAF9948624.1 | P0ABF8 | D\check{z}ugasová et al., |
| | | | 1998 |
| Fatty acyl-CoA | KAF9940771.1 | P69451, | Li et al., 2007; |
| synthetase | | P38135 | Dabirian et al., |
| | | | 2019 |
| Enzymes involved in | | | |
| PL synthesis | | | |
| Inositol-3-phosphate | KAF9950016.1 | P11986 | Feng et al., 2015 |
| synthase | | | |
| Phosphatidate | KAF9956759.1 | P0ABG1 | Shen et al., 1996; |
| cytidylyltransferase | | | Shen et al., 1997 |
| CDP-DAG synthase | KAF9291471.1 | none | Tamura et al., 2013 |
| Phosphatidylinositol | KAF9953551.1 | none | Fischl et al., 1986; |
| synthase | | | Jani et al., 2009 |
| Phosphatidylserine | KAF9960908.1 | P23830 | Delhaize et al., |
| synthase | | | 1999; Han et al., |
| | | | 2017 |
| Phosphatidylserine | KAF9951850.1 | P0A8K1 | Clancey et al., |
| decarboxylase | | | 1993; Gsell et al., |
| | | | 2013 |
| Phosphatidylserine | KAF9963219.1 | none | Trotter et al., 1995 |
| decarboxylase | | | |
| Phosphatidylethanola | KAF9280886.1 | none | Summers et al., |
| mine | | | 1988; Kodaki et al., |
| methyltransferase | | | 1989 |
| Phospholipid | KAF9947517.1 | none | Kodaki et al., 1987; |
| methyltransferase | | | McGraw et al., |
| | | | 1989 |
| Phosphatidylinositol | KAF9289337.1 | none | Ren et al., 2014 |
| transfer protein | | | |
| Phosphatidylinositol/ | KAF9282151.1 | none | Bankaitis et al., |
| phosphatidylcholine | | | 2007; Tripathi et |
| transfer protein | | | al., 2019 |
| Phosphatidylinositol | KAF9968193.1 | none | Tani et al., 2014 |
| phosphatase | | | |
| Diacylglycerol kinase | none | P0ABN1 | Han et al., 2008; |
| | | | Fakas et al., 2011 |
| Enzymes involved in | | | |
| lipid catabolism | | | |
| Cholesterol | KAF9965658.1 | none | Beopoulos et al., |
| esterase/TAG lipase | | | 2008 |
| TAG lipase | KAF9957419.1 | none | Beopoulos et al., |
| | | | 2008 |
| TAG lipase | none | none | Beopoulos et al., |
| | | | 2008; Klein et al., |
| | | | 2016 |
| TAG lipase | KAF9949630.1 | none | Klein et al., 2016 |
| Phospholipase B | KAF9960596.1 | none | Fyrst et al., 1999; |
| | | | Ferreira et al., 2018 |
| Phospholipase B | KAF9290270.1 | P0ADA1 | Lee et al, 1994; |
| | | | Ferreira et al., 2018 |
| Phospholipase D | KAF9940421.1 | none | Sreenivas et al., |
| | | | 1998 |
| Peroxisome biogenesis | KAF9964377.1 | none | Williams et al., |
| factor 10 | | | 2008 |
| Acyl-CoA oxidase | KAF9281751.1 | none | Beopoulos et al., |
| | | | 2008 |
| Acyl-CoA oxidase | KAF9927487.1 | none | Beopoulos et al., |
| | | | 2008 |
| Acyl-CoA oxidase | KAF9966479.1 | none | Beopoulos et al., |
| | | | 2008 |
| Acyl-CoA oxidase | KAF9928534.1 | none | Beopoulos et al., |
| | | | 2008 |
| Acyl-CoA oxidase | KAF9281340.1 | none | Beopoulos et al., |
| | | | 2008 |
| Acyl-CoA oxidase | none | none | Beopoulos et al., |
| | | | 2008 |
| Protein kinase | KAF9966796.1 | none | Feng et al., 2015 |
| Multifunctional- | KAF9928572.1 | none | Beopoulos et al., |
| oxidation protein | | | 2008 |
| Peroxisomal oxoacyl | KAF9274906.1 | P21151, | Beopoulos et al., |
| thiolase | | P76503 | 2008; Feng et al., |
| | | | 2015 |
| Regulators of lipid | | | |
| synthesis | | | |
| Associated with Sit4 | KAF9941704.1 | none | Luke et al., 1996 |
| protein phosphatase | | | |
| Phosphoinositide 3- | KAF9954459.1 | P23874 | Helliwell et al., |
| kinase-related protein | | | 1998 |
| kinase | | | |
| Phospholipid synthesis | KAF9965110.1 | none | Sreenivas et al., |
| regulatory protein | | | 2001; Sreenivas et |
| | | | al., 2003 |

In some embodiments, the genetic modification(s) that increase the production of saturated fatty acids in lipid reduce or prevent expression and/or activity of one or more genes in Table 1. In some embodiments, the genetic modification(s) reduce or prevent expression and/or activity of a lipid catabolism gene (see Table 1 for examples).

### Synthesis of Phospholipids in Microbes

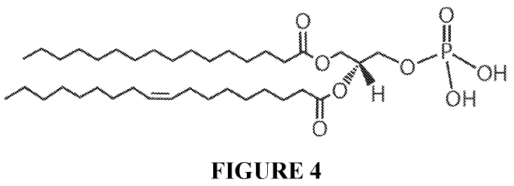

As a primary structural component of biological membranes, phospholipids play important roles in cell morphology and organelle function and some also act as secondary messengers. Phospholipids are amphipathic molecules that have a glycerol backbone esterified to a phosphate head group and two fatty acids (FIG. 4). Due to their charged headgroup at neutral pH, they are polar lipids, showing some solubility in solvents such as ethanol in addition to solvents such as chloroform. The most common fatty acids esterified to the glycerophosphate backbone of phospholipids in eukaryotic microbes such as S. cerevisiae include palmitic acid (C16:0), palmitoleic acid (C16:1), stearic acid (C18:0) and oleic acid (C18:1) (Carman and Gil-Soo, 2011). The major phospholipids found in total cell extracts from S. cerevisiae are phosphatidylcholine (PC), phosphatidylethanolamine (PE), phosphatidylinositol (PI), and phosphatidylserine (PS). Phosphatidyl glycerol (PG) and cardiolipin (CL) are minor phospholipids in total S. cerevisiae cell extracts but are the major phospholipids of mitochondrial lipids (Zhang et al., 2014). Other yeasts such as Y. lipolytica and Schizosaccharomyces pombe have a similar phospholipid make up (Fernandez et al., 1986, Fakas 2017). In contrast, the phospholipid composition of prokaryotes such as Escherichia coli is primarily comprised of PE, PG and CL and these phospholipids mainly contain the fatty acids 16:0, 16:1 and 18:1Δ11 (De Siervo, 1969). E. coli and many other bacteria lack PC.

The enzymes involved in the synthesis of phospholipids in microbes and the corresponding genes are listed in Table 1. The enzymes and genes involved in phospholipid synthesis in yeast have been characterised in detail in S. cerevisiae (Carman and Zeimetz, 1996). The specific synthesis of phospholipids begins with the synthesis of the phospholipid phosphatidic acid (PA), which is produced from glycerol-3-phosphate or dihydroxyacetone phosphate after fatty acyl coenzyme A (CoA)-dependent reactions that are catalyzed by glycerol-3-phosphate acyltransferases and the lysophospholipid acyltransferases (Athenstaedt and Daum, 1997; Athenstaedt et al., 1999; Zheng and Zou 2001). All major phospholipid classes in S. cerevisiae are synthesized from a common precursor: cytidine diphosphate diacylglycerol (CDP-DAG). CDP-DAG is synthesized in a reaction catalyzed by CDP-DAG synthase, which converts PA to CDP-DAG using cytidine triphosphate (CTP) as the CDP donor (Carter and Kennedy, 1966; Shen et al., 1996). CDP-DAG is the key intermediate for the synthesis of all of the major and minor phospholipids in S. cerevisiae as in all other yeasts. In one reaction, CDP-DAG donates its phosphatidyl moiety to inositol to form PI in the reaction catalyzed by PI synthase (Nikawa and Yamashita, 1984). The inositol used in this reaction can be derived from glucose-6-phosphate via the reactions catalyzed by inositol-3-phosphate synthase (Klig and Henry, 1984; Dean-Johnson and Henry, 1989) and inositol-3-phosphate phosphatase (Murray and Greenberg, 2000). Inositol used in the synthesis of PI can also be utilised from exogenously supplied inositol in the media by inositol permeases. CDP-DAG may also donate its phosphatidyl moiety to glycerol-3-phosphate to form phosphatidylglycerophosphate (PGP) in the reaction catalyzed by PGP synthase (Chang et al., 1998a). PGP is then dephosphorylated to PG by PGP phosphatase (Osman et al., 2010). The cardiolipin (CL) synthase catalyzes the reaction between PG and another molecule of CDP-DAG to generate CL (Chang et al., 1998b). The final enzyme that utilizes CDP-DAG is the PS synthase (Letts et al., 1983) which catalyzes the formation of PS by displacement of CMP from CDP-DAG with serine (Kanfer and Kennedy, 1964). PS is then decarboxylated to PE by PS decarboxylase enzymes (Trotter et al., 1993). PE is then converted to PC by the three-step S-adenosyl methionine (AdoMet)-dependent methylation reactions, whereby the first methylation reaction is catalyzed by the PE methyltransferase and the last two methylation reactions are catalyzed by the phospholipid methyltransferase (Kodaki and Yamashita 1987).

PE and PC can also be synthesised from exogenously supplied ethanolamine and choline by the CDP-ethanolamine and CDP-choline branches of the Kennedy pathway. The exogenously supplied ethanolamine and choline are phosphorylated by ethanolamine kinase and choline kinase with ATP to form phosphoethanolamine and phosphocholine, respectively (Kim et al., 1999; Hosaka et al., 1989). These intermediates are then activated with CTP to form CDP-ethanolamine and CDP-choline, respectively, by phosphoethanolamine cytidylyltransferase and phosphocholine cytidylyltransferase (Min-Seok et al., 1996; Tsukagoshi et al., 1987). Ethanolamine phosphotransferase and choline phosphotransferase then convert CDP-ethanolamine and CDP-choline in a reaction with DAG to form PE and PC (Hjelmstad and Bell 1988; Hjelmstad and Bell, 1991). The CTP required for the synthesis of CDP-DAG, CDP-ethanolamine, and CDP-choline is derived from UTP by the action of CTP synthetase enzymes. The DAG used for the synthesis of PE and PC via the Kennedy pathway is derived from PA by the PAH1-encoded PA phosphatase (Han et al., 2006). The DAG generated in the PA phosphatase reaction may be converted back to PA by DAG kinase (Han et al., 2008a; Han et al., 2008b) or used for the synthesis of the neutral lipid TAG by acyltransferase enzymes encoded by DGA1 and LRO1. In addition, additional acyltransferase enzymes involved in the synthesis of ergosterol esters can also acylate DAG to form TAG.

The Kennedy pathway plays a critical role in the synthesis of PE and PC when the enzymes in the CDP-DAG pathway are non-functional or defective (Carman and Henry, 1999; Greenberg and Lopes, 1996). For example, a mutant deficient in the three-step methylation of PE requires choline supplementation for growth and synthesizes PC via the CDP-choline branch of the Kennedy pathway. Mutants deficient in the synthesis of PS or PE can synthesize PC if they are supplemented with ethanolamine or choline, respectively. The ethanolamine is incorporated into PE via the CDP-ethanolamine branch of the Kennedy pathway, and the PE is subsequently methylated to form PC. Mutants defective in the CDP-DAG pathway can also synthesize PE or PC when they are supplemented with lysoPE, lysoPC, or PC with short acyl chains. LysoPE and lysoPC transported into the cell are acylated to PE and PC, respectively, by the lysophospholipid acyltransferase, which also utilizes lysoPA as a substrate. In addition, Kennedy pathway mutants defective in both the CDP-choline and CDP-ethanolamine branches can synthesize PC only by the CDP-DAG pathway. However, unlike the CDP-DAG pathway mutants the Kennedy pathway mutants do not exhibit any auxotrophic requirements and have an essentially normal complement of phospholipids.

Evidence supports that the CDP-DAG pathway is mainly responsible for the synthesis of PE and PC when cells are grown in the absence of ethanolamine and choline (Carman and Henry 1989). However, the Kennedy pathway can contribute to the synthesis of PE and PC when these precursors are not supplemented in the culture medium. For example, the PC synthesized by way of the CDP-DAG pathway is constantly hydrolyzed to choline and PA by a phospholipase D. The choline can then be incorporated back into PC via the CDP-choline branch of the Kennedy pathway, and the PA is converted to other phospholipids via the intermediates CDP-DAG and DAG.

The details provided above for S. cerevisiae phospholipid synthesis and the gene and enzymes involved are found to be also true for the oleaginous yeast Yarrowia lipolytica. Another common yeast, S. pombe, uses pathways for PL biosynthesis that are highly similar to those of S. cerevisiae. There is, however, one major difference between S. pombe and S. cerevisiae. S. pombe is a natural inositol auxotroph; it cannot grow in the absence of inositol due to the inability to form L-myoinositol 3-phosphate from its precursor glucose 6-phosphate. As a result, the PI content of S. pombe cells is strongly dependent on the concentration of inositol in the growth medium. Inositol auxotrophy of S. pombe is due to the absence of inositol-3-phosphate synthase, encoded by the INO1 gene in S. cerevisiae, as evidenced by the observation that expression of Pichia pastoris inositol-3-phosphate synthase in S. pombe can convert this natural inositol auxotroph to the inositol prototroph.

Phospholipids in E. coli and other Gram-negative bacteria are used in the construction of the inner and outer membranes. E. coli possesses only three major phospholipid species in its membranes, PE which comprises the bulk of the phospholipids (75%), with PG and CL forming the remainder, 15-20% and 5-10%, respectively. Bacterial phospholipid synthesis begins with the acylation of glycerol 3-phosphate (G3P), forming lysophosphatidic acid (lysoPA). This detergent-like intermediate undergoes a second acylation, forming phosphatidic acid (PA) which is the key precursor for bacterial phospholipids. The major PL of E. coli are synthesised from PA by the enzymes of the CDP-DAG pathway as described for S. cerevisiae. In summary, the acyltransfer module deposits PA in the membrane, where it is activated to CDP-DAG by CDP-DAG synthase. This intermediate is used for both PE synthesis via PS synthase and PS decarboxylase (Psd). PG is formed from the same intermediate by PGP synthase and the phosphorylated intermediate is dephosphorylated by PGP phosphatase. Finally, CL is produced by the condensation of two PG molecules by CL synthase.

### Microbial Cells

A wide variety of different microbial cells can be used in the present invention. In an embodiment the microbial cells exist as single celled organisms, however such cells may clump together. Examples of microbial cells of the invention include bacterial cells and eukaryotic cells such as fungal cells and algal cells. Eukaryotic microbes are preferred over bacterial (prokaryotic) microbes. As used herein, the terms “microbial cell”, “microbe” and “microorganism” mean the same thing.

In an embodiment, the microbial cells are suitable for fermentation, although they can also be cultured under ambient oxygen concentrations. In another embodiment, the microbial cells are oleaginous cells, preferably an oleaginous eukaryotic microbe, or preferably derived from a progenitor oleaginous microbe such as a progenitor eukaryotic oleaginous microbe. In another embodiment, microbial cells are heterotrophic cells, preferably a heterotrophic eukaryotic microbe. The microbial cells preferably have at least two of these, more preferably are characterised by all of these features.

In an embodiment, the cells of the invention are yeast cells. Examples of yeast cells useful for the invention include, but are not limited to, Saccharomyces sp. such as Saccharomyces cerevisiae, Yarrowia sp. such as Yarrowia lipolytica, Pichia sp. such as Pichia pastoris, Candida sp. such as Candida rugosa, Aspergillus sp. such as Aspergillus niger, Cryptococcus sp. such as Cryptococcus curvatus, Lipomyces sp. such as Lipomyces starkeyi, Rhodosporidium sp. such as Rhodosporidium toruloides, Rhodotorula sp. such as Rhodotorula glutinis and Trichosporon sp. such as Trichosporon fermentans.

In an embodiment, the fungal cells are mold cells. Examples of mold cells useful for the invention include, but are not limited to, Cunninghamella sp. such as Cunninghamella echinulate, Mortierella sp. such as Mortierella isabellina or Mortierella alpina, Mucorales sp. such as Mucorales fungi and Trichoderma sp. such as Trichoderma harzianum.

In an embodiment, the cells are bacterial cells. Examples of bacterial cells useful for the invention include, but are not limited to, Acinetobacter such as Acinetobacter baylyi, Alcanivorax sp. such as Alcanivorax borkumensis, Gordonia sp. such as DG, Mycobacterium sp. such as Mycobacterium tuberculosis, Nocardia sp. such as Nocardia globerula, Rhodococcus sp. such as Rhodococcus opacus, and Streptomyces sp. such Streptomyces coelicolor.

In an embodiment, the cells are algal cells such as microalgal, or Bacillariophyceae, cells. Examples of algal cells useful for the invention include, but are not limited to, Prototheca sp. such as Prototheca moriformis, Thraustochytrium spp., Chlorella sp. such as Chlorella protothecoides, Chlorella vulgaris or Chlorella ellipsoidea, Schizochytrium sp. such as Schizochytrium strain FCC-1324, Dunaliella sp., Haematococcus sp. such as Haematococcus pluvialis, Neochloris sp. such as Neochloris oleabundans such as strain UTEX #1185, Pseudochlorococcum sp., Scenedesmus sp. such as Scenedesmus obliquus, Tetraselmis sp. such as Tetraselmis chui or Tetraselmis tetrathele, Chaetoceros sp. such as Chaetoceros calcitrans, Chaetoceros gracilis or Chaetoceros muelleri, Nitzschia sp. such as Nitzschia cf. pusilla, Phaeodactylum sp. such as Phaeodactylum tricornutum, Skeletonema sp. such as strain CS 252, Thalassiosira sp. such as Thalassiosira pseudonana, Crypthecodinium sp. such as Crypthecodinium cohnii, Isochrysis sp. such as Isochrysis zhangjiangensis, Nannochloropsis sp. such as Nannochloropsis oculata such as strain NCTU-3, Pavlova sp. such as Pavlova salina, Rhodomonas sp. and Thalassiosira sp. such as Thalassiosira weissflogii.

### Polypeptides

The terms “polypeptide” and “protein” are generally used interchangeably.

A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970; GCG program) with parameters of a gap creation penalty=5, and a gap extension penalty=0.3. The query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over the entire length of the reference amino acid sequence. The polypeptide or class of polypeptides may have the same enzymatic activity as, or a different activity than, or lack the activity of, the reference polypeptide. Preferably, the polypeptide has an enzymatic activity of at least 10%, at least 50%, at least 75% or at least 90%, of the activity of the reference polypeptide.

A polynucleotide defined herein may encode a biologically active fragment of an enzyme such as an acyltransferase or a thioesterase. As used herein a “biologically active” fragment is a portion of a polypeptide defined herein which maintains a defined activity of a full-length reference polypeptide, for example possessing acyltransferase or thioesterase activity or other enzyme activity. Biologically active fragments as used herein exclude the full-length polypeptide. Biologically active fragments can be any size portion as long as they maintain the defined activity. Preferably, the biologically active fragment maintains at least 10%, at least 50%, at least 75% or at least 90%, of the activity of the full-length protein.

With regard to a defined polypeptide or enzyme, it will be appreciated that % identity figures higher than those provided herein will encompass preferred embodiments. Thus, where applicable, in light of the minimum % identity figures, it is preferred that the polypeptide/enzyme comprises an amino acid sequence which is at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO. In an embodiment, for each of the ranges listed above, the % identity does not include 100% i.e. the amino acid sequence is different to the nominated SEQ ID NO.

Amino acid sequence variants/mutants of the polypeptides of the defined herein can be prepared by introducing appropriate nucleotide changes into a nucleic acid defined herein, or by in vitro synthesis of the desired polypeptide. Such variants/mutants include, for example, deletions, insertions or substitutions of residues within the amino acid sequence. A combination of deletion, insertion and substitution can be made to arrive at the final construct, provided that the final peptide product possesses the desired enzyme activity.

Mutant (altered) peptides can be prepared using any technique known in the art. For example, a polynucleotide defined herein can be subjected to in vitro mutagenesis or DNA shuffling techniques as broadly described by Harayama (1998). Products derived from mutated/altered DNA can readily be screened using techniques described herein to determine if they possess, for example, acyltransferase or thioesterase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites which are not conserved amongst naturally occurring proteins such as acyltransferases or a thioesterases. These sites are preferably substituted in a relatively conservative manner in order to maintain enzyme activity. Such conservative substitutions are shown in Table 2 under the heading of “exemplary substitutions”.

In a preferred embodiment a mutant/variant polypeptide has only, or not more than, one or two or three or four conservative amino acid changes when compared to a naturally occurring polypeptide. Details of conservative amino acid changes are provided in Table 2. As the skilled person would be aware, such minor changes can reasonably be predicted not to alter the activity of the polypeptide when expressed in a recombinant cell.

TABLE 2
Exemplary substitutions.

| Original Residue | Exemplary Substitutions |
|---|---|
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

### Polynucleotides

The invention also provides for the use of polynucleotides which may be, for example, a gene, an isolated polynucleotide, or a chimeric genetic construct such as a chimeric DNA. It may be DNA or RNA of genomic or synthetic origin, double-stranded or single-stranded, and combined with carbohydrate, lipids, protein or other materials to perform a particular activity defined herein. The term “polynucleotide” is used interchangeably herein with the term “nucleic acid molecule”.

In an embodiment, the polynucleotide is non-naturally occurring. Examples of non-naturally occurring polynucleotides include, but are not limited to, those that have been codon optimised for expression in microbial cell, those that have been mutated, for example by using methods described herein, and polynucleotides where an open reading frame encoding a protein is operably linked to a promoter to which it is not naturally associated, for example as in the constructs described herein, i.e a promoter that is heterologous with respect to the open reading frame.

As used herein, a “chimeric DNA” or “chimeric genetic construct” or similar refers to any DNA molecule that is not a native DNA molecule in its native location, also referred to herein as a “DNA construct”. Typically, a chimeric DNA or chimeric gene comprises regulatory and transcribed or protein coding sequences that are not found operably linked together in nature i.e. that are heterologous with respect to each other. Accordingly, a chimeric DNA or chimeric gene may comprise regulatory sequences and coding sequences that are derived from different sources, or regulatory sequences and coding sequences derived from the same source, but arranged in a manner different than that found in nature.

An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA technology. The terms “foreign polynucleotide” or “exogenous polynucleotide” or “heterologous polynucleotide” and the like refer to any nucleic acid which is introduced into the genome of a cell by experimental manipulations. Foreign or exogenous genes may be genes that are inserted into a non-native organism, native genes introduced into a new location within the native host, or chimeric genes. A “transgene” is a gene that has been introduced into the genome by a transformation procedure. The terms “genetic modification”, “genetic variation”, “transgenic” and variations thereof include introducing genes into cells by transformation or transduction, mutating genes in cells, deleting genes, and altering or modulating the regulation of a gene by a heritable change in the genome in a cell or organism to which these acts have been done or their progeny. A “genomic region” as used herein refers to a position within the genome where a transgene, or group of transgenes (also referred to herein as a cluster), have been inserted into a cell, or an ancestor thereof. Such regions only comprise nucleotides that have been incorporated by the intervention of a human such as by methods described herein.

The term “exogenous” in the context of a polynucleotide refers to the polynucleotide when present in a cell in an altered amount compared to its native state. In one embodiment, the cell is a cell that does not naturally comprise the polynucleotide. However, the cell may be a cell which comprises a non-endogenous polynucleotide resulting in an altered amount of production of the encoded polypeptide. An exogenous polynucleotide includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically such chimeric polynucleotides comprise at least an open reading frame encoding a polypeptide operably linked to a promoter suitable of driving transcription of the open reading frame in a cell of interest.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO. In an embodiment, for each of the ranges listed above, the % identity does not include 100% i.e. the nucleotide sequence is different to the nominated SEQ ID NO.

Polynucleotides may possess, when compared to naturally occurring molecules, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid as described above). It is thus apparent that polynucleotides can be either from a naturally occurring source or recombinant. Preferred polynucleotides are those which have coding regions that are codon-optimised for translation in microbial cells, as is known in the art.

### Recombinant Vectors

Recombinant expression can be used to produce genetically modified microbes of the invention. Recombinant vectors contain heterologous polynucleotide sequences, that is, polynucleotide sequences that are not naturally found adjacent to polynucleotide molecules defined herein that preferably are derived from a species other than the species from which the polynucleotide molecule(s) are derived. The vector can be either RNA or DNA and typically is a plasmid. Plasmid vectors typically include additional nucleic acid sequences that provide for easy selection, amplification, and transformation of the expression cassette in prokaryotic cells, e.g., pYES-derived vectors, pUC-derived vectors, pSK-derived vectors, pGEM-derived vectors, pSP-derived vectors, or pBS-derived vectors. Suitable yeast expression vectors include the pPIC series of vectors, yeast integrating plasmids (YIp), yeast replicating plasmids (YRp), yeast centromere plasmids (YCp), and yeast episomal plasmids (YEp). Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of microbial cells. The recombinant vector may comprise more than one polynucleotide defined herein, for example three, four, five or six polynucleotides defined herein in combination, preferably a chimeric genetic construct described herein, each polynucleotide being operably linked to expression control sequences that are operable in the cell.

“Operably linked” as used herein refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence, such as a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance. For example, an intron in a 5′ UTR sequence or towards the 5′ end of a protein coding region can contain a transcriptional enhancer, providing an increased expression level, for example an FBAIN promoter region.

To facilitate identification of transformants, the nucleic acid construct desirably comprises a selectable or screenable marker gene as, or in addition to, the foreign or exogenous polynucleotide. By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked. The actual choice of a marker is not crucial as long as it is functional (i.e., selective) in combination with the cells of choice.

Examples of selectable markers are markers that confer antibiotic resistance such as hygromycin, nourseothricin, ampicillin, erythromycin, chloramphenicol or tetracycline resistance, preferably hygromycin or kanamycin resistance.

Recombinant yeast of the invention may comprise a reporter gene which either encodes a galactosidase or a selectable growth marker.

The “galactosidase” may be any enzyme which cleaves a terminal galactose residue(s) from a variety of substrates, and which is able to also cleave a substrate to produce a detectable signal. In an embodiment, the galactosidase is a β-galactosidase such as bacterial (for instance from E. coli) LacZ. In an alternate embodiment, the galactosidase is an α-galactosidase such as yeast (for instance S. cerevisiae) Mel-1. β-galactosidase activity may be detected using substrates for the enzyme such as X-gal (5-bromo-4-chloro-indolyl-β-D-galactopyranoside) which forms an intense blue product after cleavage, ONPG (o-nitrophenyl galactoside) which forms a water soluble yellow dye with an absorbance maximum at about 420 nm after cleavage, and CPRG (chlorophenol red-β-D-galactopyranoside) which yields a water-soluble red product measurable by spectrophotometry after cleavage. α-galactosidase activity may be detected using substrates for the enzyme such as o-nitrophenyl α-D-galactopyranoside which forms an indigo dye after cleavage, and chlorophenol red-α-D-galactopyranoside which yields a water-soluble red product measurable by spectrophotometry after cleavage. Kits for detecting galactosidase expression in yeast are commercially available, for instance the β-galactosidase (LacZ) expression kit from Thermo Scientific.

Preferably, the selectable growth marker is a nutritional marker or antibiotic resistance marker.

Typical yeast selectable nutritional markers include, but are not limited to, LEU2, TRP1, HIS3, HIS4, URA3, URA5, SFA1, ADE2, MET15, LYS5, LYS2, ILV2, FBA1, PSE1, PDI1 and PGK1. Those skilled in the art will appreciate that any gene whose chromosomal deletion or inactivation results in an unviable host, so called essential genes, can be used as a selective marker if a functional gene is provided on the, for example, plasmid, as demonstrated for PGK1 in a pgkl yeast strain. Suitable essential genes can be found within the Stanford Genome Database (SGD) (http:://db.yeastgenome.org). Any essential gene product (e.g. PDI1, PSE1, PGK1 or FBA1) which, when deleted or inactivated, does not result in an auxotrophic (biosynthetic) requirement, can be used as a selectable marker on a, for example, plasmid in a yeast host cell that, in the absence of the plasmid, is unable to produce that gene product, to achieve increased plasmid stability without the disadvantage of requiring the cell to be cultured under specific selective conditions. By “auxotrophic (biosynthetic) requirement” we include a deficiency which can be complemented by additions or modifications to the growth medium.

### Expression

Expression vectors can direct gene expression in microbial cells. As used herein, an expression vector is a vector that is capable of transforming a host cell and of effecting expression of one or more specified polynucleotide molecule(s). Expression vectors useful for the invention contain regulatory sequences such as transcription control sequences, translation control sequences, origins of replication, and other regulatory sequences that are compatible with the recombinant cell and that control the expression of polynucleotide molecules of the present invention. In particular, polynucleotides or vectors useful for the present invention include transcription control sequences. Transcription control sequences are sequences which control the initiation, elongation, and termination of transcription. Particularly important transcription control sequences are those which control transcription initiation, such as promoter and enhancer sequences. Suitable transcription control sequences include any transcription control sequence that can function in at least one of the recombinant cells of the present invention. The choice of the regulatory sequences used depends on the target microbial cell. A variety of such transcription control sequences are known to those skilled in the art.

Yeast cells are typically transformed by chemical methods (e.g., as described by Rose et al., 1990, Methods in Yeast Genetics, Cold Spring Harbor Laboratory Press, Cold Spring Harbor, N.Y., and in Kawai et al., 2010). The cells are typically treated with lithium acetate to achieve transformation efficiencies of approximately 10⁴ colony-forming units (transformed cells)/μg of DNA. Other standard procedures for transforming yeast include i) the spheroplast method which, as the name suggests, relies on the production of yeast spheroplasts, ii) the biolistic method where DNA coated metal microprojectiles are shot into the cells, and iii) the glass bead methods which relies on the agitation of the yeast cells with glass beads and the DNA to be delivered to the cell. Of course, any suitable means of introducing nucleic acids into yeast cells can be used.

It is well known that transformation of organisms, such as yeast, with exogenous plasmids can lead to clonal differences in the penetrance of the transformed gene, due to differences in copy number or other factors. It is therefore advisable to screen two or more independent clonal isolates for each transformed receptor in order to maximise the likelihood of identifying suitable receptor=ligand pairs during screening. Different clonal isolates may be screened independently or may be combined into a single well for screening. The latter option may be particularly convenient where a nutritional reporter is used rather than a colorimetric reporter.

“Constitutive promoter” refers to a promoter that directs expression of an operably linked transcribed sequence in the cell without the need to be induced by specific growth conditions. Examples of constitutive promoters useful for yeast cells of the invention include, but are not limited to, a yeast PGK (phosphoglycerate kinase) promoter, a yeast ADH-1 (alcohol dehydrogenase) promoter, a yeast ENO (enolase) promoter, a yeast glyceraldehyde 3-phosphate dehydrogenase promoter (GPD) promoter, a yeast PYK-1 (pyruvate kinase) promoter, a yeast translation-elongation factor-1-alpha promoter (TEF) promoter and a yeast CYC-1 (cytochrome c-oxidase promoter) promoter. In a preferred embodiment, a yeast promoter is a S. cerevisiae promoter. In another embodiment, the constitutive promoter may not have been derived from yeast. Examples of such promoters useful for the invention include, but are not limited to, the cauliflower mosaic virus 35S promoter, the glucocorticoid response element, and the androgen response element. The constitutive promoter may be the naturally occurring molecule or a variant thereof comprising, for example, one, two or three nucleotide substitutions which do not abolish (and preferably enhance) promoter function.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules defined herein include, but are not limited to, integration of the polynucleotide molecule into one or more host cell chromosomes, addition of stability sequences to mRNAs, substitutions or modifications of transcription control signals (e.g., promoters, operators, enhancers), substitutions or modifications of translational control signals (e.g., ribosome binding sites, Shine-Dalgarno sequences), modification of polynucleotide molecules to correspond to the codon usage of the host cell, and the deletion of sequences that destabilize transcripts.

### Other Genetic Modification Techniques

Any method can be used to introduce a nucleic acid molecule into a microbial cell and many such methods are well known. For example, transformation and electroporation are common methods for introducing nucleic acid into yeast cells (see, e.g., Gietz et al., 1992; Ito et al., 1983; Becker et al., 1991).

In an embodiment, the integration of a gene of interest into a specific chromosomal site in a microbial cell occurs via homologous recombination. According to this embodiment, an integration cassette containing a module comprising at least one marker gene and/or the gene to be integrated (internal module) is flanked on either side by DNA fragments homologous to those of the ends of the targeted integration site (recombinogenic sequences). After transforming the microbial cell with the cassette by appropriate methods, a homologous recombination between the recombinogenic sequences may result in the internal module replacing the chromosomal region in between the two sites of the genome corresponding to the recombinogenic sequences of the integration cassette (Orr-Weaver et al., 1981).

In an embodiment, the integration cassette for integration of a gene of interest into a microbial cell includes the heterologous gene under the control of an appropriate promoter together with a selectable marker flanked by recombinogenic sequences for integration of a heterologous gene into the microbial cell chromosome. In an embodiment, the heterologous gene includes any of the fatty acid biosynthesis genes described herein.

Where deletion of an endogenous gene is desired, the integration cassette can comprise a selectable marker (without any other heterologous gene sequence) flanked by DNA fragments homologous to those of the ends (and/or neighbouring sequences) of the endogenous gene targeted for deletion. Other methods suitable for deleting or mutating endogenous genes (e.g., using site-specific or RNA-guided nucleases) are described below.

The selectable marker gene can be any marker gene used in microbial cells, including but not limited to, HIS3, TRP1, LEU2, URA3, bar, ble, hph, and kan. The recombinogenic sequences can be chosen at will, depending on the desired integration site suitable for the desired application.

In another embodiment, integration of a gene into the chromosome of the microbial cell may occur via random integration (Kooistra et al., 2004).

Additionally, in an embodiment, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke et al., 1984).

The exogenous nucleic acid molecule contained within a microbial cell of the disclosure can be maintained within that cell in any form. For example, exogenous nucleic acid molecules can be integrated into the genome of the cell or maintained in an episomal state that can stably be passed on (“inherited”) to daughter cells. Such extra-chromosomal genetic elements (such as plasmids, mitochondrial genome, etc.) can additionally contain selection markers that ensure the presence of such genetic elements in daughter cells. Moreover, the microbial cells can be stably or transiently transformed. In addition, the microbial cells described herein can contain a single copy, or multiple copies of a particular exogenous nucleic acid molecule as described above.

### Genome Editing Using Site-Specific Nucleases

Genome editing uses engineered nucleases composed of sequence specific DNA binding domains fused to a non-specific DNA cleavage module. These chimeric nucleases enable efficient and precise genetic modifications (including deletions, mutations and insertions) by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption.

Engineered nucleases useful in the methods of the present invention include zinc finger nucleases (ZFNs) and transcription activator-like (TAL) effector nucleases (TALEN).

Typically nuclease encoded genes are delivered into cells by plasmid DNA, viral vectors or in vitro transcribed mRNA. The use of fluorescent surrogate reporter vectors also allows for enrichment of ZFN- and TALEN-modified cells. As an alternative to ZFN gene-delivery systems, cells can be contacted with purified ZFN proteins which are capable of crossing cell membranes and inducing endogenous gene disruption.

A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell. Typically, a ZFN having more than three zinc fingers would have progressively greater specificity with each additional zinc finger.

The zinc finger domain can be derived from any class or type of zinc finger. In a particular embodiment, the zinc finger domain comprises the Cis₂His₂ type of zinc finger that is very generally represented, for example, by the zinc finger transcription factors TFIIIA or Spl. In a preferred embodiment, the zinc finger domain comprises three Cis₂His₂ type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques. (see, for example, Bibikova et al., 2002).

The ZFN DNA-cleavage domain is derived from a class of non-specific DNA cleavage domains, for example the DNA-cleavage domain of a Type II restriction enzyme such as FokI (Kim et al., 1996). Other useful endonucleases may include, for example, HhaI, HindIII, Nod, BbvCI, EcoRI, BglI, and AlwI.

In order to target genetic recombination or mutation according to a preferred embodiment of the present invention, two 9 bp zinc finger DNA recognition sequences must be identified in the host microbial cell DNA. These recognition sites will be in an inverted orientation with respect to one another and separated by about 6 bp of DNA. ZFNs are then generated by designing and producing zinc finger combinations that bind DNA specifically at the target locus, and then linking the zinc fingers to a DNA cleavage domain.

ZFN activity can be improved through the use of transient hypothermic culture conditions to increase nuclease expression levels (Doyon et al., 2010) and co-delivery of site-specific nucleases with DNA end-processing enzymes (Certo et al., 2012). The specificity of ZFN-mediated genome editing can be improved by use of zinc finger nickases (ZFNickases) which stimulate HDR without activation the error-prone NHE-J repair pathway (Kim et al., 2012; Wang et al., 2012; Ramirez et al., 2012; McConnell Smith et al., 2009).

A transcription activator-like (TAL) effector nuclease (TALEN) comprises a TAL effector DNA binding domain and an endonuclease domain.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

Genome editing using programmable RNA-guided DNA endonucleases Distinct from the site-specific nucleases described above, the clustered regulatory interspaced short palindromic repeats (CRISPR)/Cas system provides an alternative to ZFNs and TALENs for inducing targeted genetic alterations. In bacteria, the CRISPR system provides acquired immunity against invading foreign DNA via RNA-guided DNA cleavage.

CRISPR systems rely on CRISPR RNA (crRNA) and transactivating chimeric RNA (tracrRNA) for sequence-specific silencing of invading foreign DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

CRISPR loci are a distinct class of interspersed short sequence repeats (SSRs) that were first recognized in E. coli (Ishino et al., 1987; Nakata et al., 1989). Similar interspersed SSRs have, been identified in Haloferax mediterranei, Streptococcus pyogenes, Anabaena, and Mycobacterium tuberculosis (Groenen et al., 1993; Hoe et al., 1999; Masepohl et al., 1996; Mojica et al., 1995).

The CRISPR loci differ from other SSRs by the structure of the repeats, which have been termed short regularly spaced repeats (SRSRs) (Janssen et al., 2002; Mojica et al., 2000). The repeats are short elements that occur in clusters, that are always regularly spaced by unique intervening sequences with a constant length (Mojica et al., 2000). Although the repeat sequences are highly conserved between strains, the number of interspersed repeats and the sequences of the spacer regions differ from strain to strain (van Embden et al., 2000).

The common structural characteristics of CRISPR loci are described in Jansen et al. (2002) as (i) the presence of multiple short direct repeats, which show no or very little sequence variation within a given locus; (ii) the presence of non-repetitive spacer sequences between the repeats of similar size; (iii) the presence of a common leader sequence of a few hundred basepairs in most species harbouring multiple CRISPR loci; (iv) the absence of long open reading frames within the locus; and (v) the presence of one or more cas genes.

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

As used herein, the term “cas gene” refers to one or more cas genes that are generally coupled associated or close to or in the vicinity of flanking CRISPR loci. A comprehensive review of the Cas protein family is presented in Haft et al. (2005). The number of cas genes at a given CRISPR locus can vary between species.

### Cell Culture

Effective culture conditions are known to those skilled in the art and include, but are not limited to, suitable media, bioreactor, temperature, pH and oxygen conditions that permit lipid production. A suitable medium refers to any medium in which a cell is cultured to produce lipid defined herein. Such medium typically comprises an aqueous medium having assimilable carbon, nitrogen and phosphate sources, and appropriate salts, minerals, metals and other nutrients, such as vitamins. Cells defined herein can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Lipid Extraction

Extraction of the lipid from microbial cell of the invention uses analogous methods to those known in the art for lipid extraction from oleaginous microorganisms, such as for example described in Patel et al. (2018). In one embodiment, the extraction is performed by solvent extraction where an organic solvent (e.g., hexane or a mixture of hexane and ethanol) is mixed with at least the biomass, preferably after the biomass is dried and ground, but it can also be performed under wet conditions. The solvent dissolves the lipid in the cells, which solution is then separated from the biomass by a physical action (e.g., ultrasonication). Ultrasonication is one of the most extensively used pretreatment methods to disrupt the cellular integrity of microbial cells. Other pretreatment methods can include treatment with acid such a sulphuric acid, microwave irradiation, high-speed homogenization, high-pressure homogenization, bead beating, autoclaving, and thermolysis. For example, treatment with 2% sulphuric acid ate 60° C. for 5 min. The organic solvent can then be separated from the non-polar lipid (e.g., by distillation). This second separation step yields non-polar lipid from the cells and can yield a re-usable solvent if one employs conventional vapor recovery.

In solvent extraction, an organic solvent (e.g., hexane or a mixture of hexane and ethanol) is mixed with at least the biomass of the microbial cell, preferably after the biomass is dried and ground. The solvent dissolves the lipid in the biomass and the like, which solution is then separated from the biomass by mechanical action (e.g., with the processes above). This separation step can also be performed by filtration (e.g., with a filter press or similar device) or centrifugation etc. The organic solvent can then be separated from the non-polar lipid (e.g., by distillation). This second separation step yields non-polar lipid from the microbial cell and can yield a re-usable solvent if one employs conventional vapor recovery.

The lipid extracted from the microbial cells of the invention may be subjected to normal oil processing procedures. As used herein, the term “purified” when used in connection with lipid of the invention typically means that that the extracted lipid has been subjected to one or more processing steps of increase the purity of the lipid component. For example, a purification step may comprise one or more or all of the group consisting of: degumming, deodorising, decolourising, drying and/or fractionating the extracted oil. However, as used herein, the term “purified” does not include a transesterification process or other process which alters the fatty acid composition of the lipid or oil of the invention so as to change the fatty acid composition of the total fatty acid content. Expressed in other words, in a preferred embodiment the fatty acid composition of the purified lipid is essentially the same as that of the unpurified lipid.

### Degumming

Degumming is an early step in the refining of lipids in a liquid form (oil) and its primary purpose is the separation of most of the phospholipids from the oil, which may be present as approximately 1-2% of the total extracted lipid. Addition of ˜2% of water, typically containing phosphoric acid, at 70-80° C. to the crude oil results in the separation of most of the phospholipids accompanied by trace metals and pigments. The insoluble material that is removed is mainly a mixture of phospholipids and is also known as lecithin. Degumming can be performed by addition of concentrated phosphoric acid to the crude extracted lipid to convert non-hydratable phosphatides to a hydratable form, and to chelate minor metals that are present. Gum is separated from the oil by centrifugation. The recovered gum comprising ω6 fatty acids, other than LA alone, is encompassed in the present invention.

### Alkali Refining

Alkali refining is one of the refining processes for treating lipid in the form of an oil, sometimes also referred to as neutralization. It usually follows degumming and precedes bleaching. Following degumming, the oil can treated by the addition of a sufficient amount of an alkali solution to titrate all of the fatty acids and phosphoric acids, and removing the soaps thus formed. Suitable alkaline materials include sodium hydroxide, potassium hydroxide, sodium carbonate, lithium hydroxide, calcium hydroxide, calcium carbonate and ammonium hydroxide. This process is typically carried out at room temperature and removes the free fatty acid fraction. Soap is removed by centrifugation or by extraction into a solvent for the soap, and the neutralised oil is washed with water. If required, any excess alkali in the oil may be neutralized with a suitable acid such as hydrochloric acid or sulphuric acid.

### Bleaching

Bleaching is a refining process in which oils are heated at 90-120° C. for 10-30 minutes in the presence of a bleaching earth (0.2-2.0%) and in the absence of oxygen by operating with nitrogen or steam or in a vacuum. This step in oil processing is designed to remove unwanted pigments and the process also removes oxidation products, trace metals, sulphur compounds and traces of soap.

### Deodorization

Deodorization is a treatment of oils and fats at a high temperature (200-260° C.) and low pressure (0.1-1 mm Hg). This is typically achieved by introducing steam into the oil at a rate of about 0.1 ml/minute/100 ml of oil. After about 30 minutes of sparging, the oil is allowed to cool under vacuum. The oil is typically transferred to a glass container and flushed with argon before being stored under refrigeration. This treatment improves the colour of the oil and removes a majority of the volatile substances or odorous compounds including any remaining free fatty acids, monoacylglycerols and oxidation products.

### Esterification and Transesterification

As used herein, “esterification” refers to a chemical reaction that forms at one least fatty acid ester through an esterification reaction between a fatty acid and an alcohol. Fatty acid esters (FAEs) are a type of ester that result from the combination of a fatty acid with an alcohol. When the alcohol component is glycerol, the fatty acid esters produced can include monoglycerides, diglycerides, or triglycerides.

As used herein, “transesterification” means a process that exchanges the fatty acids within and between TAGs (interesterification) or transfers the fatty acids to another alcohol to form an ester. This may initially involve releasing fatty acids from the TAGs as free fatty acids or it may directly produce fatty acid esters, preferably fatty acid methyl esters or ethyl esters. In a transesterification reaction of the TAG with an alcohol such as methanol or ethanol, the alkyl group of the alcohol forms an ester linkage with the acyl groups (including the SCFA) of the TAG.

### Food, Feedstuffs, Beverages and Compositions

The present invention includes lipids and compositions which can be used as a food or beverage for human consumption or a feedstuff for animal consumption, preferably at least a food for human consumption, or as food or beverage ingredients which may be used to prepare the food or beverage. The compositions can also be added to a food, beverage or feedstuff to improve one or more of the texture, the appearance, the aroma and/or flavour of the food, beverage or feedstuff. The lipids may be used to improve the consistency or processing ability of doughs, for example, or to increase the consistency, texture or taste of a food when warmed above room temperature. For purposes of the present invention, a food, beverage or feedstuff is a preparation for human or animal consumption which when taken into the body (a) serve to nourish or build up tissues or supply energy; and/or (b) maintain, restore or support adequate nutritional status or metabolic function.

Suitable foods/feedstuffs include meat substitutes, soup bases, stew bases, snack foods, bouillon powders, bouillon cubes, flavour packets, or frozen food products. Meat substitutes can be formulated, for example, as hot dogs, burgers, ground meat, sausages, steaks, filets, roasts, breasts, thighs, wings, meatballs, meatloaf, bacon, strips, fingers, nuggets, cutlets, or cubes.

The lipid of the invention can be used as a food ingredient as a fat, or in oils and dressings, or in products such as butter, powdered butter, margarine, mayonnaise or salad dressing. It can be used in soups such as, for example, canned soup or ready-to-eat soup, a noodle bowl or noodle cup product, stews, stocks, broths, canned vegetables, dehydrated vegetables. It can be used in sauces and gravies, pasta sauces, tomato products, dry seasoning mixes, seasoning cubes. It can be used in bakery products such as, for example, bread, bread substitutes, pastries, croissants, biscuits, savoury biscuits, crackers, cakes, pizza dough, pie pastry, dry bakery mixes, bakery dough. Specific examples include, for example, muffins (e.g., English muffins), crackers (e.g., salted crackers, baked crackers, graham crackers, etc.), rolls (e.g., soft rolls, dinner rolls, crescent rolls), biscuits (e.g., buttermilk biscuits, cobbler biscuits), pie crusts, breads (e.g., focaccia, bruschetta, sourdough breads, soda breads, breadsticks, corn bread), pizza doughs, bagels. Sweet dough can be used to prepare brownies, cookies, muffins, turnovers, doughnuts, cakes, pastries, pies, scones, and the like. It can be used in mixed ingredient dishes such as, for example, frozen and canned meals. It can be used in snacks such as sweet snacks, savoury food products or salty snacks including, for example, potato chips, crisps, nuts, tortilla-tostada, pretzels, cheese snacks, corn snacks, potato-snacks, ready-to-eat popcorn, microwaveable popcorn, pork rinds, nuts, crackers, cracker snacks, breakfast cereals, meats, cured meats, luncheon/breakfast meats, peanut butter. It can be used in dairy product substitutes and analogues such as, for example, ice cream, ice cream desserts, frozen yoghurt, milk, fresh/pasteurized milk, full fat fresh/pasteurized milk, semi skimmed fresh/pasteurized milk, long-life/UHT milk, full fat long life/UHT milk, semi skimmed long life/UHT milk, fat-free long life/UHT milk, goat milk, condensed/evaporated milk, plain condensed/evaporated milk, flavoured, functional and other condensed milk, flavoured milk drinks, dairy only flavoured milk drinks, soy milk, sour milk drinks, fermented dairy drinks, coffee creamers/whiteners, powder milk, flavoured powder milk drinks, cream, yoghurt, plain/natural yoghurt, flavoured yoghurt, fruited yoghurt, probiotic yoghurt, yoghurt drinks, and other dairy-based desserts. It can be used in other food products such as breakfast cereals, cereal flakes, muesli, children's breakfast cereals and hot cereals.

A food, beverage or feedstuff of the invention comprises, for example, extracted lipid of the invention, the microbial cell of the invention, the microbial cell extract or the composition of the invention. The food may either be in a solid or liquid form. Additionally, the composition may include edible macronutrients, protein, carbohydrate, vitamins, and/or minerals in amounts desired for a particular use. The amounts of these ingredients will vary depending on whether the composition is intended for use with normal individuals or for use with individuals having specialized needs, such as individuals suffering from metabolic disorders and the like.

Examples of suitable ingredients with nutritional value include, but are not limited to, macronutrients such as edible fats, carbohydrates and proteins. Examples of such edible fats other than the lipids of the invention include, but are not limited to, coconut oil, borage oil, fungal oil, black current oil, soy oil, and mono- and diglycerides. Examples of such carbohydrates include (but are not limited to): glucose, edible lactose, and hydrolyzed starch. Additionally, examples of proteins which may be utilized in the nutritional composition of the invention include (but are not limited to) soy proteins, electrodialysed whey, electrodialysed skim milk, milk whey, or the hydrolysates of these proteins.

With respect to vitamins and minerals, the following may be added to the food, beverage or feedstuff of the present invention: calcium, phosphorus, potassium, sodium, chloride, magnesium, manganese, iron, copper, zinc, selenium, iodine, and Vitamins A, E, D, C, and the B complex. Other such vitamins and minerals may also be added.

Additional ingredients include food-grade oils such as canola, corn, sunflower, soybean, olive or coconut oil, seasoning agents such as edible salts (e.g., sodium or potassium chloride) or herbs (e.g., rosemary, thyme, basil, sage, or mint), flavouring agents, proteins (e.g., soy protein isolate, wheat glutin, pea vicilin, and/or pea legumin), protein concentrates (e.g., soy protein concentrate), emulsifiers (e.g., lecithin), gelling agents (e.g., k-carrageenan or gelatin), fibers (e.g., bamboo filer or inulin), or minerals (e.g., iodine, zinc, and/or calcium).

Foods and feedstuffs described herein also can include a natural coloring agent such as turmeric or beet juice, or an artificial coloring agent such as azo dyes, triphenylmethanes, xanthenes, quinines, indigoids, titanium dioxide, red #3, red #40, blue #1, or yellow #5.

Foods and feedstuffs described herein also can include meat shelf life extenders such as carbon monoxide, nitrites, sodium metabisulfite, Bombal, vitamin E, rosemary extract, green tea extract, catechins and other anti-oxidants.

The components utilized in the food, beverage or feedstuff of the present invention can be of semi-purified or purified origin. By semi-purified or purified is meant a material which has been prepared by purification of a natural material or by de novo synthesis.

In an embodiment, the food, beverage or feedstuff has no components derived from an animal. Thus, in a preferred embodiment, at least some of the ingredients are plant material or material derived from a plant. In some embodiments, the food, beverage or feedstuff can be soy-free, wheat-free, yeast-free, MSG-free, and/or free of protein hydrolysis products, and can taste meaty, highly savory, and without off odors or flavours or reduced levels thereof.

In addition, compositions of the invention can be used to modulate the taste, texture, appearance, mouth-feel and/or aroma profile of other food products (e.g., meat replicas, meat substitutes, tofu, mock duck or other gluten based vegetable product, textured vegetable protein such as textured soy protein, pork, fish, lamb, or poultry products such as chicken or turkey products) and can be applied to the other food product before or during cooking.

In some embodiments, the compositions described herein comprise components required for causing a Maillard reaction upon heating the composition. For example, the composition may comprise one or both of (i) a sugar, sugar alcohol, sugar acid, or sugar derivative, and (ii) and an amino acid or derivative thereof.

Suitable sugars, sugar alcohols, sugar acids, and sugar derivatives include glucose, fructose, ribose, sucrose, arabinose, glucose-6-phosphate, fructose-6-phosphate, fructose 1,6-diphosphate, inositol, maltose, molasses, maltodextrin, glycogen, galactose, lactose, ribitol, gluconic acid and glucuronic acid, amylose, amylopectin, or xylose.

Suitable amino acids and derivatives thereof include cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate, glutamine, glycine, proline, serine, and tyrosine.

The composition may also comprise another one or more other flavour precursors including oils (e.g., vegetable oils), free fatty acids, α-hydroxy acids, dicarboxylic acids, nucleosides, nucleotides, vitamins, peptides, protein hydrolysates, extracts, phospholipids, lecithin, and organic molecules.

Foods, feedstuffs, beverages and compositions described herein can be packaged in various ways, including being sealed within individual packets or shakers, such that the composition can be sprinkled or spread on top of a food product before or during cooking.

Foods, beverages and feedstuffs described herein can be assessed for texture, appearance, mouth-feel, flavour and aroma using trained human panelists. The evaluations can involve eyeing, feeling, chewing, smelling and tasting of the product to judge product appearance, color, integrity, texture, flavour, and mouth feel, etc, of the food, beverage or feedstuff. Panelists can be served samples under red or under white light. A scale can be used to rate the overall acceptability or quality of the food or specific quality attributes such meatiness, texture, and flavour.

In some embodiments, a food, beverage or feedstuff described herein can be compared to another product (e.g., meat or meat substitute) based upon olfactometer readings. In various embodiments, the olfactometer can be used to assess odor concentration and odor thresholds, odor suprathresholds with comparison to a reference gas, hedonic scale scores to determine the degree of appreciation, or relative intensity of odors.

In some embodiments, volatile chemicals identified using GCMS can be evaluated. For example, a human can rate the experience of smelling the chemical responsible for a certain peak. This information could be used to further refine the profile of flavour and aroma compounds produced by the compositions of the present invention.

Characteristic flavour and fragrance components are mostly produced during the cooking process by chemical reactions molecules including amino acids, fats and sugars which are found in plants as well as meat. Therefore, in some embodiments, a food, beverage or feedstuff is tested for similarity to meat during or after cooking. In some embodiments human ratings, human evaluation, olfactometer readings, or GC-MS measurements, or combinations thereof, are used to create an olfactory map of the food or feedstuff. Similarly, an olfactory map of the food, beverage or feedstuff, for example, a meat replica, can be created. These maps can be compared to assess how similar the cooked food or feedstuff is to meat.

### Personal Care Products

The lipid of the invention may be used in personal care products such as pharmaceuticals, cosmetics and toiletries. Examples include aftershave lotions, baby lotions, oils, powders and creams, baby shampoos, basecoats and undercoats, bath capsules, bath oils, tablets and salts, bath soaps and detergents, beard softeners, blushers, body and hand preparations, bubble baths, cleaning products, colognes and toilet waters, cuticle softeners, dentifrices, deodorants, depilatories, douches, dressings, eye lotions, eye makeup preparations miscellaneous, eye makeup removers, eye shadows, eyebrow pencils, eyeliners, face and neck preparations, face powders, feminine hygiene deodorants, foot powders and sprays, foundations, fragrance preparations, hair bleaches, hair colour sprays, hair colouring preparations miscellaneous, hair conditioners, hair dyes and colours, hair lighteners with colour, hair preparations, hair rinses, hair shampoos, hair sprays, hair tints, hair wave sets, hair grooming aids, indoor tanning preparations, leg and body paints, lipsticks, lozenges, makeup bases, makeup fixatives, makeup preparations, manicuring preparations, mascara, moisturising preparations, mouthwashes and breath fresheners, nail creams and lotions, nail extenders, nail polish and enamels, night skin care preparations, oral care products, oral hygiene products, paste masks, perfumes, personal cleanliness products, preshave lotions, rouges, sachets, shampoos, shaving cream, shaving preparations, shaving soap, skin care preparations miscellaneous, skin fresheners, suntan gels, creams and liquids, suntan preparations, toothpastes, tooth gels, tooth whitening products and tonics.

### Examples

### Example 1. Materials and Methods

### Media

YPD medium is a rich medium which contains 10 g/L yeast extract (Sigma Aldrich, Catalog No. Y1625), 20 g/L peptone (Sigma Aldrich, Catalog No. P0556) and 20 g/L glucose (Sigma Aldrich, Catalog No. G7021). YPD plates contain, in addition, 20 g/L agar. SD-Ura medium contained Yeast Synthetic Drop-out Medium (Sigma Catalog No. Y1501). This medium was supplemented with uracil when required.

### Media for Larger Scale Cultures

Unless otherwise stated, the medium used for preparing seed cultures for larger scale cultures (2 L or more) was a defined medium (DM-Gluc), having a base medium (BM) containing 10.64 g/L potassium di-hydrogen orthophosphate (KH₂PO₄), 4.0 g/L di-ammonium hydrogen orthophosphate ((NH₄)₂HPO₄) and 1.7 g/L citric acid (monohydrate). These ingredients were dissolved in about 70% of the required volume of water that had been purified by reverse osmosis, adjusted to pH 6.0 with 2 M NaOH, and made up to the required volume using purified water. The BM was sterilised at 121° C. for 20 min and cooled to room temperature. The following ingredients were then added separately: 30 ml/L of 660 g/L glucose (autoclaved), to a final concentration of 20 g/L, 10 ml/L 1 M magnesium sulphate heptahydrate (autoclaved), 10 ml/L Trace metal solution (see below, filter sterilised), 10 ml/L 15 g/L thiamine hydrochloride (filter sterilised), 3 ml/L 10% (v/v) Sigma Antifoam 204 (autoclaved).

The fermentation medium (FM) for 2 L and 10 L cultures also used the BM as base medium. The required volume was added to the bioreactor and sterilised at 121° C. for a 60 min fluid cycle for an autoclavable bioreactor or 30 min for a steam-in-place bioreactor, and cooled to 31° C. The following ingredients were added, per litre of base medium: 121 ml/L of 660 g/L glucose (autoclaved), giving a final concentration of 80 g/L, 5 ml/L of 1M magnesium sulphate heptahydrate (autoclaved), 5 ml/L of Trace metal solution (see below, filter sterilised), 5 ml/L 15 g/L thiamine hydrochloride (filter sterilised) and 50 ml/L of 200 g/L ammonium chloride (filter sterilised). The glucose, magnesium, trace metal solution and thiamine solution were mixed and added to the bioreactor together. Once the medium was formulated, the pH was checked, normally slightly less than 6.0. A pH controller was used to add ammonia solution to the medium and bring the pH to 6.0.

Small scale (50 ml) and larger scale cultures of 2 L or more were also grown in a defined medium containing glycerol at 8% (w/v) and having a lower nitrogen content (DM-Glyc-LowN) for inducing more TAG synthesis. This medium was the same as DM-Gluc except that the glucose was replaced with 80 g/L glycerol (final concentration) as carbon source and the (NH₄)₂HPO₄ content was reduced to 0.5 g/L. For the larger cultures, starter cultures were grown in SD-Ura medium, with addition of uracil and any amino acids if required, for 24-48 h. A sample of the starter culture was centrifuged and the cells used to inoculate the larger culture. These cultures were incubated for 48-96 h and the pH maintained at 6.0 unless otherwise stated.

Alternative culture media were the same as DM-Glyc-LowN except that they contained either glucose or glycerol at 8% (w/v) as the carbon source, the (NH₄)₂HPO₄ was replaced with (NH₄)₂SO₄ at a final concentration of 0.5 g/L to provide a low nitrogen level, yeast extract was added at 1 g/L, and the MgSO₄ and the citric acid were omitted. These media are referred to herein as DM-Gluc-LowN-LowMg and DM-Glyc-LowN-LowMg, respectively.

The Trace metal solution (TM) used in the media described above contained, per litre: 2.0 g CuSO₄·5H₂O, 0.08 g NaI, 3.0 g MnSO₄·H₂O, 0.2 g NaMoO₄·2H₂O, 0.02 g H₃BO₃, 0.5 g CoCl₂·6H₂O, 7.0 g ZnCl₂, 22.0 g FeSO₄·7H₂O, 0.50 g CaSO₄·2H₂O, and 1 ml of sulphuric acid. The reagents were added in the listed order. Addition of the sulphuric acid resulted in dissolution of the calcium sulphate. The trace metal solution was filtered sterilised through a 0.2 μm filter and stored at 2-8° C. in a bottle wrapped in aluminium foil.

One pH control reagent was a phosphoric acid solution (10% w/v), prepared by adding 118 ml of 85% H₃PO₄ to 882 ml of purified water. The solution was sterilised by autoclaving.

The other was an ammonia solution (10% v/v), prepared by adding 330 ml of a 30% ammonia solution to 670 ml of purified water. That solution was assumed to be self-sterilising. An antifoam solution was prepared by mixing 100 ml of Sigma antifoam 204 with 900 ml of purified water, providing a concentration of 10%. The mixture was sterilised by autoclaving.

A feed solution was prepared by adding 134 ml of 200 g/L ammonium chloride which had been filter sterilised to 1 L of 660 g/L glucose, and sterilised by autoclaving.

### Microbial Strains and Cloning Vectors

S. cerevisiae strains INVSc1 (ThermoFisher, Catalog No. C81000) and D5A (ATCC 200062) were used as host strains for experiments on production of lipids including phospholipids. When testing various lipid modification genes in yeast by addition of transgenes, the pYES2 plasmid was used as the base vector for introduction of the genes. INVSc1 and pYES2 were obtained from Invitrogen (Catalog No. V825-20). The genotype of INVSc1 was: MATa his3Δ1 leu2 trp1-289 ura3-52/MATa his3Δ1 leu2 trp1-289 ura3-52, and its phenotype was: His-, Leu-, Trp- and Ura-. The pYES2 vector had unique HindIII and AhoI restriction enzyme sites which were used for insertion of DNA fragments encoding various proteins as described herein. The pYES2 expression vector contained a URA3 gene as a selectable marker gene for introduction into yeast strains that were Ura-, a 2p origin of replication for high copy maintenance, and an inducible Gall promoter for expression of the protein coding regions in yeast. The plasmid also contained an ampicillin resistance gene for selection in E. coli during cloning experiments.

Several strains of Yarrowia lipolytica were obtained from the American Type Culture Collection (Manassas VA, USA): Strain JM23 (ATCC 90812) having the genotype leu2-35 lys5-12 ura3-18 xpr2::LYS5B, strain IFP29 (ATCC 20460) having the genotype leu2-35 lys5-12 ura3-18 xpr2::LYS5B, and wild-type strain W29 (Casaregola et al., 2000).

Escherichia co/i strains DH5a and BL21 were obtained from ThermoFisher Scientific (Catalog Nos. 18265017, EC0114).

Growth of S. cerevisiae and Y. lipolytica Cultures for Lipid Analysis

To provide an inoculum for cultures for fatty acid production, extraction and analysis, small-scale cultures of Y. lipolytica or S. cerevisiae were grown in 5 ml of YPD medium at 29° C. for 24 h. For experiments, the inoculum culture was diluted into the growth medium having a volume of, for example, 50-2000 ml to an optical density at 600 nm (OD600) of 0.1. Cultures were grown in polypropylene tubes for 10 ml cultures, or glass flasks for larger volumes, the container having a volume at least 5-fold greater than the culture volume. The containers were sealed with 3M micropore surgical tape (Catalog No. 1530-1) tape and incubated in a shaker at a defined temperature of 29° C. unless specified otherwise, at 200 rpm for aeration.

When SD-Ura medium was used, a carbon source such as 2% glycerol or raffinose (w/v) (MP Chemicals, USA, Catalog No. 4010022) was used. Cultures were incubated overnight at 28° C. with shaking for aeration. The inoculum culture was diluted into 10 ml of SD-Ura medium, or other volume as specified, containing 2% (w/v) glycerol or raffinose to provide an initial OD600 of 0.1. The culture in a 50 ml tube or a 250 ml flask was incubated in a shaker at 28° C. at 200 rpm for aeration. The OD600 was checked at time intervals of 15 or 30 min. When the OD600 reached 0.3, exogenous compounds as potential substrates (if any) were added along with 2% galactose for induction of the transgene from the GAL1 promoter if appropriate.

Larger scale cultures of S. cerevisiae cells at a volume of 3 L were grown for transformants such as pYES2 derivatives. These were inoculated from glycerol stocks. Starter cultures were grown in 10 ml SD-Ura medium containing 2% (w/v) raffinose for up to 48 h. The cells were transferred into 3 L of SD-Ura medium containing 2% (w/v) raffinose to an OD600 of 0.1 and grown at 28° C. with shaking at 200 rpm. The OD600 was checked at time intervals of 15 and 30 min. When the OD600 reached 0.3, galactose was added to a final concentration of 2% (w/v) to induce the transgene. When desired, sodium butyrate was added to cultures to a final concentration of 2 mg/ml or sodium stearate at 0.5 mg/L unless otherwise stated. The flasks were then closed loosely with sterile aluminium foil. The cultures were grown in the incubator for 48 hours before harvesting the cells by centrifugation.

Cultures of E. coli were grown from glycerol stocks in 5 ml LB medium for 24 h to provide an inoculum. The culture was diluted into LB medium in polypropylene tubes or glass flasks, to an OD600 of 0.1 and incubated in a shaker at 37° C. at 200 rpm for aeration, unless otherwise specified.

### Feeding Lipid Substrates to the Cells

For substrate feeding experiments, both yeast and bacterial inoculum cultures were diluted into their respective growth media containing 1% tergitol (Sigma Aldrich Catalog No. NP40S) at an OD600 of 0.1 and incubated with shaking for a period of time, typically 2 h. Lipid substrates such as e.g. fatty acids, oil or oil-hydrolysates were then added to the medium and the cultures further incubated for different time periods. Fatty acid substrates were dissolved in ethanol and provided to the cultures to a final concentration of 0.5 mg/ml, or the sodium salts of the fatty acids were provided in aqueous solution.

When compounds were added to the media as potential carbon sources or substrates (feeding assays), the following compounds were obtained from Sigma Aldrich: ethanolamine (Catalog No. 110167), choline chloride (C7017), myo-inositol (13011), butyric acid (B103500), sodium butyrate (B5887), tributyrin (W222305) orpalmitic acid (76119).

### Parameters for 2 L Fermentation

The following parameters were used for a 3 L (total volume) Sartorius Biostat B autoclavable bioreactor with a maximum working volume of 2 L culture. The starting medium volume was 1 L. The initial temperature set point was 31° C., unchanged for the duration of the process. The temperature controller configuration was Minimum: −100%; Maximum: 100%; XP: 4%; TI: 300 sec; TD: 75 sec; Dead: 0.0%; Cascade control using dissolved oxygen controller; Minimum agitator speed: 500 rpm; Maximum agitator speed: 1200 rpm; pH control set point: 6.0; pH controller configuration: Minimum: −100%, Maximum: 100%, XP: 30%, TI 30 sec, TD: 0 sec, Dead: 0.2% (equivalent to 0.02 pH units). The acid and base used for automated pH control were 10% H₃PO₄ and 10% ammonia solution.

The initial dissolved oxygen set point was 30%. The dissolved oxygen (DO) electrode was calibrated after sterilisation and once the medium temperature had stabilised at 31° C. 0% saturation was calibrated using pure nitrogen, a stirrer speed of 100 rpm and nitrogen flow rate at 0.1 L/min, and saturation was established with the stirrer speed set at 500 rpm and air flow rate at 0.5 L/min. For cascade control, a two step cascade used a stirrer followed by gas mix to provide oxygen enrichment of the air flow. Oxygen enrichment was used to reduce the air flow rates and thereby reduce foaming which can have a negative impact on the process, since the yeast cells tended to float on the foam. The airflow was constant at 0.5 L/min, with minimum oxygen enrichment at 0% and maximum oxygen enrichment at 50%. The dissolved oxygen controller configuration was set at: Dead: 0%, Minimum: 0% (510 rpm), Maximum: 100% (1425 rpm), XP: 90%, TI: 50 sec, TD: 0 sec.

For foam control, automatic chemical foam control was achieved with 10% Sigma Antifoam 204, adding 10 ml of 10% (v/v) Sigma Antifoam 204 before inoculation, 20 ml at 7 h post inoculation, and 30 ml added 31 h post inoculation. The foam controller configuration was: Cycle: 10 sec, Pulse: 5 sec, Sensitivity: 04.

The target inoculation OD600 was 0.20, calculated based on the starting volume of base medium, using the secondary seed culture. For fed batch mode, feed with the feed solution commenced 14 h after inoculation with a feed flow rate of 20 ml/h. At the completion of each process, the vessel was drained, and the cells were harvested by centrifugation.

### Parameters for 10 L Fermentation

The same parameters were used for a 15 L Sartorius Biostat C10 steam-in-place bioreactor with a maximum working volume of 10 L culture, with the following differences. To calibrate the dissolved oxygen (DO) electrode, 0% saturation was calibrated using pure nitrogen at a stirrer speed 100 rpm and nitrogen flow rate of 1 L/min, and saturation was established with the stirrer speed set at 500 rpm and air flow rate at 3 L/min. For cascade control, the airflow was constant at 3.0 L/min. The dissolved oxygen controller configuration was set at: HTime Stirrer: 0 min, Dead: 0.5%, Minimum: 34% at 510 rpm, Maximum: 95% at 1425 rpm, XP: 150%, TI: 100 sec, TD: 0 sec, HTime GasMix: 0 min, Dead: 0.5%, Minimum: 0% (no oxygen supplementation), Maximum: 50%, XP: 5%, TI: 200 sec, TD: 0 sec.

As for the 2 L fermentation, the target inoculation OD600 was 0.20, using a secondary seed culture. For fed batch mode, feed with the feed solution commenced 14 h after inoculation with a feed flow rate of 100 ml/h. At the completion of each process, 24 h after inoculation unless otherwise stated, the culture was heat inactivated at 105° C. for 5 min, then cooled to 31° C. before harvesting the cells by centrifugation.

### Seed Culture for Larger Scale Cultures

For a primary seed culture, a frozen glycerol stock of the yeast strain was used to inoculate 100 mL of DM in a plastic baffled 1 L Erlenmeyer flask with a vented cap. This was incubated at 28° C. with shaking at 200 rpm for aeration for 24±2 h. The optical density at 600 nm (OD600) was measured at the end of incubation. A secondary seed culture was prepared by using the primary seed culture to inoculate 500 mL of DM in a plastic baffled 2 L Erlenmeyer flask with a vented cap, to a starting OD600 of 0.04. The second seed culture was incubated at 28° C. with shaking at 200 rpm for 16±2 hours. The OD600 was measured at the end of incubation. This culture was used to inoculate the large-scale fermentation.

### Cell Harvesting, Washing and Freeze Drying

Cells from smaller scale cultures were harvested by centrifugation, for example in a 50 ml tube at 4600 g for 15 min, washed twice with 10 ml and finally washed with 1 ml MilliQ water. For the final wash, where a dry cell weight was to be measured, the cell suspension was transferred to a pre-weighed 2 ml Eppendorf tube, centrifuged, and the cell pellet freeze-dried (VirTis Bench Top freeze dryer, SP Scientific) before weighing and lipid extraction. When lipid substrates such as ARA, DGLA, γ-linolenic acid (GLA), butyrate or palmitate were added to the growth medium, cell pellets were washed successively with 1 ml of 1% tergitol (v/v), 1 ml of 0.5% tergitol and a final wash with 1 ml water to remove any remaining substrate from the exterior of the cells and freeze-dried as described above. When an oil was added to the growth medium, cells were harvested by centrifugation as above but the cell pellets were washed successively with 5 ml of 10% tergitol (v/v), 5 ml of 5% tergitol, 5 ml of 1% tergitol, 5 ml of 0.5% tergitol and a final wash with 5 ml water to remove any remaining oil from the exterior of the cells. In some cases, microscopic observation after staining with Bodipy confirmed the absence of oil stained at the cell walls. With the final wash, pellets were transferred to pre-weighed 2 ml Eppendorf tubes and freeze-dried before weighing and lipid extraction.

Lipid Extraction from Yeast Cells

Total cellular lipid was extracted from yeast cells such as S. cerevisiae or Y. lipolytica by using a method modified from Bligh and Dyer (1959). Approximately 50 mg freeze-dried cells were homogenized with 0.6 ml of a mixture of chloroform/methanol (2/1, v/v) with 0.5 g zirconium oxide beads (Catalog No. ZROB05, Next Advance, Inc., USA) in a 2 ml Eppendorf tube using a Bullet Blender Blue (Next Advance, Inc. USA) at speed 6 for 5 min. The mixture was then sonicated in an ultrasonication water bath for 5 min and 0.3 ml 0.1 M KCl was added. The mixture was shaken for 10 min and centrifuged at 10,000 g for 5 min. The lower, organic phase containing lipid was transferred to a glass vial and remaining lipid was extracted from the upper phase containing the cell debris by mixing it with 0.4 ml chloroform for 20 min and centrifugation. The lower phase was collected and combined with the first extract in the glass vial. The solvent was evaporated from the lipid sample under a flow of nitrogen gas and the extracted lipid resuspended in a measured volume of chloroform. If required, the lipid samples were stored at −20° C. until further analysis.

Lipid Extraction from the Larger Biomass

For the extraction of total lipid from a larger biomass, a different method of cell homogenization was used with larger volumes of the solvents, unless otherwise stated. Approximately 1.5 g of freeze-dried cells, distributed amongst six 50 ml Cellstar polypropylene tubes (6× Tube A) (Catalog No. 227261, Greiner bio-one) was homogenized in 9 ml chloroform/methanol (2/1, v/v) per tube using an Ultra-Turrax T25 homogenizer (IKA Labortechnik Staufen, Germany) for 3 min. Further homogenization was carried out for 2 min after adding 3 ml 1 M KCl to each tube. Each tube was centrifuged at 6,000 g for 3 min. The lower phase was transferred to a new tube (Tube B) and the solvent was evaporated under a flow of nitrogen at room temperature. The upper phase was mixed with 1 g of glass beads in a Vibramax mixer for 10 min and with vigorous vortexing for 1 min. 6 ml chloroform was added to each tube and mixed again for 3 min. After centrifugation, the lower phase was transferred to Tube B and the solvent was evaporated under a flow of nitrogen gas at room temperature. To extract remaining lipid, the upper phase in Tube A was mixed with another 6 ml chloroform and mixed for 3 min. After centrifugation, the lower phase was again transferred to Tube B. 3 ml methanol and 3 ml 0.1 M KCl were added to Tube B and mixed for 3 min. The lower phase was transferred to a Falcon tube and the solvent was evaporated under a flow nitrogen gas at room temperature. The extracted lipid was dissolved in chloroform/methanol (2/1, v/v) and stored at −20° C.

### Lipid Fractionation by Thin Layer Chromatography

To separate different lipid types such as TAG, DAG, free fatty acid and polar lipids such as phospholipids (PL), total lipids were fractionated on thin layer chromatography (TLC) plates (Silica gel 60; Catalog No. 1.05626.0001, MERCK, Darmstadt, Germany) using hexane:diethylether:acetic acid (70/30/1 v/v/v) as the solvent system. A sample of a lipid standard such as 18-6A containing TAG, DAG, FFA and MAG (Nu-Chek Prep Inc, USA) was run in an adjacent lane to identify the different lipid spots. When distinguishing different TAGs containing short-chain fatty acids (SCFA), a standard containing triheptadecanoin (Nu-chek, USA, Catalog No. T-155), a triglyceride mix C2-C10 containing equal amounts of triacetin (TAG 6:0), tributyrin (TAG 12:0), tricaprillin (TAG 18:0) and tridecanoin (TAG 30:0) (Sigma Aldrich, Catalog No 17810-1AMP-S) were run in adjacent lanes to identify the TAG lipid spots. After the chromatography, the plates were sprayed with a primuline (Catalog No. 206865, Sigma, Taufkirchen, Germany) solution prepared at a concentration of 5 mg/100 ml in acetone:water (80/20 v/v) and lipid bands visualised under UV light. The silica with the lipid from each spot was scraped off and transferred to a tube. The lipid fractions were extracted from the silica for derivatisation using either methylation, propylation or butylation.

Larger Scale Fractionation of PL and TAG from Total Lipid

PL and TAG were fractionated from about 100 mg of total lipid by loading the lipid on 18 cm lines on each of eight TLC plates (Silica gel 60; Catalog No. 1.05626.0001, MERCK, Darmstadt, Germany) and chromatographed with a solvent mixture consisting of hexane/diethylether/acetic acid (70:30:1, v:v:v). An aliquot of a lipid standard containing TAG, DAG, FFA and MAG (18-6Δ; NuChek Inc, USA) was run in parallel to assist with identifying the lipid bands. After staining the plates with primuline and visualisation under UV light, the PL bands located at the origin and the TAG bands having the same mobility as the TAG standard were collected and transferred to Falcon tubes. The lipid/silica samples were extracted with a mixture of 6 ml chloroform and 3 ml methanol, mixing vigorously for 5 min, then adding 3 ml water and further mixing for 5 min. After centrifugation for 5 min at 3,000 g, the lower organic phase was transferred to a new tube. The lower phase was transferred to a Falcon tube after centrifugation at 3000 rcf for 5 min. The upper phase was mixed with 5 ml chloroform for 5 min to extract any remaining lipid. After centrifugation, the lower phase was combined with the first extract. The solvent was evaporated under a flow of nitrogen gas. The extracted lipid, TAG or PL, was dissolved in a small volume of chloroform and filtered through 0.2 μm micro-spin filter (Chromservis, EU, Catalog No. CINY-02) to remove any particulates. The fatty acid composition and amount of each PL and TAG fraction were determined by preparation of FAME and GC analysis. Such preparations were used, for example, to separate different polar lipid classes such as PC, PE, PI and PS, or in Maillard reactions for aroma tests or for detection of volatile compounds as reaction products.

### Lipid Derivatisation to Fatty Acid Methyl Esters (FAME)

For analysis by GC, fatty acid methyl esters (FAME) were prepared from total extracted lipid or the purified TAG or PL fractions by treatment with 0.7 ml 1 N methanolic-HCl (Sigma Aldrich, Catalog No. 90964) in a 2 ml glass vial having a PTTE-lined screw cap at 80° C. for 2 h. A known amount of heptadecanoin (Nu-Chek Prep, Inc., Catalog No. N-7-A, Waterville, MN, USA) dissolved in toluene was added to each sample before the treatment as an internal standard for quantification. After the vials were cooled, 0.3 ml of 0.9% NaCl (w/v) and 0.1 ml hexane were added and the mixtures vortexed for 5 min. The mixture was centrifuged at 1700 g for 5 min and the upper, hexane phase containing the FAME was analysed by GC.

### Analysis and Quantification of FAME by GC

The individual FAMEs were identified and quantified by GC using an Agilent 7890A GC (Palo Alto, California, USA) with a 30 m SGE-BPX70 column (70% cyanopropyl polysilphenylene-siloxane, 0.25 mm inner diameter, 0.25 m film thickness), a split/splitless injector and an Agilent Technologies 7693 Series auto sampler and injector, and a flame ionisation detector (FID). Samples were injected in split mode (50:1 ratio) at an oven temperature of 150° C. The column temperature was programmed for 150° C. for 1 min, increasing to 210° C. at 3° C./min, holding for 2 min and reaching 240° C. at 50° C./min, then holding at 240° C. for 0.4 min. The injector temperature was set at 240° C. and the detector at 280° C. Helium was used as the carrier gas at a constant flow of 1.0 ml/min. FAME peaks were identified based on retention times of FAME standards (GLC-411, GLC-674; NuChek Inc., USA). Peaks were integrated with Agilent Technologies ChemStation software (Rev B.04.03 (16), Palo Alto, California, USA) based on the response of the known amount of the external standard GLC-411 (NuChek) and C17:0-ME internal standard. The resultant data provide the fatty acid composition on a weight basis, with percentages of each fatty acid (weight %) in a total fatty acid content of 100%. These percentages on a weight basis could readily be converted to percentages on a molar basis (mol %) based on the known molecular weight of each fatty acid.

### Saponification of Triacylglycerols

Free fatty acids were released from TAG by incubating 1 mg TAG in 0.2 ml 3 M KOH for 3 min at 80° C. After cooling the sample to room temperature, 100 μl hexane was added to the mixture. The mixture was vortexed for 5 min, centrifuged at 1700 g for 5 min and the upper organic phase collected for GC analysis.

### Lipid Derivatisation to Ethyl Esters or Propyl Esters

To convert the fatty acids in TAG to fatty acid ethyl esters (FAEE), 2 mg of TAG was incubated in 1N HCl/ethanol solution at 80° C. for 2 h. After cooling the sample to room temperature, 100 μl hexane was added to the mixture. The mixture was vortexed for 5 min, centrifuged at 1700 g for 5 min and the upper organic phase collected for GC analysis. To convert the fatty acids in TAG to fatty acid propyl esters, 2 mg of TAG was incubated in 1N HCl/propanol rather than 1N HCl/ethanol and otherwise processed the same.

### Peak Identity by GC-MS

The identities of unknown or uncertain peaks in the GC-FID chromatograms were confirmed by Gas Chromatography Mass Spectrometry (GC-MS) analysis. Samples were run on a GC-MS operating in the Electron Ionization mode at 70 eV to confirm peak identities and to identify possible extra peaks corresponding to possible contamination, degradation products or reagent signals. A Shimadzu GC-MS QP2010 Plus (Shimadzu Corporation, Japan) system coupled to an HTX-Pal liquid auto-sampler was used with the following parameters: 1 or 2 μl injection volume using a split/splitless inlet at a 15:1 split, at a temperature of 250° C. The oven temperature program used was the same as for the GC-FID. MS ion source and interface temperatures were 200° C. and 250° C., respectively. Data were collected at a scan speed of 1000 and scan range from 40 to 500 m/z. Peak separation was provided by a Stabilwax or Stabilwax-DA (Restek/Shimadzu) capillary column (30 m×0.25 mm i.d., 0.25 μm film thickness) using He as a carrier gas at 30 cm/sec. Mass spectra correlations were performed using a NIST library, retention indices and matching retention time of available standards. Identified SCFA was set to be present when S/N ratio were above 10:1. Instrument blanks and procedural blanks were run for quality control purposes.

### Recombinant DNA Methods

Derivatives of pYES2 having single genes inserted for testing in S. cerevisiae were made by inserting protein coding regions between the unique HindIII and XhoI sites or other restriction enzyme sites in the plasmid as appropriate by standard cloning methods. The E. coli strain DH5a was used for cloning and plasmid propagation and DNA preparation according to standard methods.

The GoldenGate (GG) method (Larroude et al., 2018) allows for rapid and efficient combinatorial assembly of multiple expression cassettes in a single vector and was therefore used to make multigene constructs for testing in S. cerevisiae or Y. lipolytica. GG DNA parts and donor vectors, also called LO vectors, according to Celinska et al. (2017) and Larroude et al. (2018) were obtained from Addgene, USA. The DNA parts included promoters (GGE146, GGE151 and GGE294), terminators (GGE014, GGE015, GGE080, GGE020 and GGE021) and the backbone assembly vector (destination vector) was GGE114.

Protein coding regions for insertion into the vectors by GG assembly were codon optimised for S. cerevisiae or Y. lipolytica using Twist Bioscience and GeneArt online software (Twist Bioscience: www.twistbioscience.com/products/genes; ThermoFisher/GeneArt: www.thermofisher.com/au/en/home/life-science/cloning/gene-synthesis/geneart-gene-synthesis.html) and synthesised either by Twist Bioscience or GeneArt (ThermoFisher, USA), or in the lab. Internal BsaI restriction enzyme sites were avoided in the codon optimised nucleotide sequences of the protein coding regions as BsaI sites were used in the GG assembly method. NotI restriction enzyme sites were also avoided within the nucleotide sequences as NotI was used for linearizing the genetic constructs for transformation of Y. lipolytica. When one, two or three genes were to be inserted into a single vector, the individual components were designed with 4-nucleotide overhangs immediately 5′ of each translation start codon (ATG) and 3′ of the translation stop codon, with the sequence of each 4-nucleotide overhang depending on the position of the component in the backbone vector GGE114, according to Table 3. The external BsaI site with the appropriate 4-nt overhang was added to the 5′ end of each DNA strand. The 4-nt overhang sequences used between consecutive components can be varied so that any 4-nt sequence can be used at each site to allow joining between the consecutive components, as known in the art.

The protein coding regions were synthesised in a cloning vector having a kanamycin selection marker gene to avoid any false positives when performing the GG reaction with the GG backbone vector GGE114 which had an ampicillin selectable marker gene. The E. coli strain DH5a was used for cloning and plasmid propagation according to standard methods. Antibiotics were used as appropriate for selecting transformed cells, for example ampicillin was added at 100 μg/mL for selection of constructs having an ampicillin selectable marker gene.

The destination vector GGE114 contained the red fluorescence protein (RFP) chromophore, which acts as a colour-based visual marker for negative cloning in E. coli, as described by Larroude et al. (2018). The vector GGE114 was a so-called destination vector that, in addition to a bacterial replicon, contained ZETA sequences for more efficient integration into the Y. lipolytica genome and the URA3 selectable marker gene with a view to reducing the number of fragments to assemble when employing this combination, into the backbone vector pYES2 which contained a 2p origin for high-copy maintenance. In this case, the RFP was between the URA3 marker gene and the ZETA down. In the presence of BsaI enzyme, the RFP was released and the one, two or three transcription units (TU; promoter-protein coding region-terminator) were inserted.

TABLE 3
Nucleotide sequences of overhangs used in
GoldenGate cloning method

| | | |
|---|---|---|
| Position | 5′ overhang | 3′ overhang |

One gene insertion

| | | |
|---|---|---|
| Promoter 1 | ACGG | AATG |
| Gene 1 | AATG | TCTA |
| Terminator 1 | TCTA | GAGT |

Two gene insertion

| | | |
|---|---|---|
| Promoter 1 | ACGG | AATG |
| Gene 1 | AATG | TCTA |
| Terminator 1 | TCTA | GCTT |
| Promoter 2 | GCTT | ACAA |
| Gene 2 | ACAA | GGAT |
| Terminator 2 | GGAT | GAGT |

Three gene insertion

| | | |
|---|---|---|
| Promoter 1 | ACGG | AATG |
| Gene 1 | AATG | TCTA |
| Terminator 1 | TCTA | GCTT |
| Promoter 2 | GCTT | ACAA |
| Gene 2 | ACAA | GGAT |
| Terminator 2 | GGAT | GTCA |
| Promoter 3 | GTCA | CCAC |
| Gene 3 | CCAC | GTAT |
| Terminator 3 | GTAT | GAGT |

The GG assembly reaction mixes contained equimolar quantities (50 ng) of the GG backbone vector such as GGE114 and other DNA components (donor vectors) in a final volume of 7.5 μl, by adding 0.75 μl 10× T4 ligase buffer, 0.75 μl 10×BSA (bovine serum albumin), 0.75 μl BsaI HF-V2 (NEB), 0.5 d T4 ligase (NEB). The reaction mixtures were incubated with 25 cycles of 37° C. for 3 min followed by 16° C. for 4 min, then 1 cycle of 50° C. for 5 min and 80° C. for 5 min. Samples of 2-3 μl were introduced into competent cells of E. coli strain DH5a by standard methods. Colonies lacking the RFP were confirmed to contain the desired genetic inserts by colony PCR with the appropriate primers and verified with restriction digests. Glycerol stocks were made and stored at −80° C.

Transformation of S. cerevisiae

A rapid method was used for introduction into S. cerevisiae of genetic constructs based on pYES2 which did not use competent cells. A loop full of S. cerevisiae cells was scraped off a fresh plate and the cells resuspended in 100 μl of transformation buffer (Sigma Aldrich, Catalog No. T0809). About 1 μg of plasmid DNA with 10 μl of 10 mg/ml salmon testes DNA which had been boiled for 5 min prior to use were added to the cell suspension along with 600 μl of plate buffer (Sigma Aldrich, Catalog No. P8966) and mixed well. The mixture was incubated at room temperature in a rotor wheel at the lowest speed for 16 hours. The mixture was then heat shocked for 15 min at 42° C., spun at 3500 rpm for 3 min, and the pellet of cells resuspended in 200 μl of sterile water. Aliquots of up to 100 μl were plated out onto synthetic drop-out selection media lacking uracil (SD-URA, Sigma Aldrich, Catalog No. Y1501) for selection of transformants. The plates were incubated at 28° C. for 3 days or until colonies appeared. Two or more colonies were picked from each plate and tested for the presence of the genetic construct by colony PCR to identify transformants.

Transformation of Y. lipolytica for Integration of Expression Cassettes

DNA of genetic constructs which included the expression cassettes (transcription units) for insertion into Y. lipolytica by homologous recombination was digested with NotI or other appropriate restriction enzymes to release the expression cassette. The linearised DNA was introduced into competent cells of the selected Ura-Y. lipolytica strain or other recipient strain as desired, prepared using the Frozen-EZ Yeast Transformation II kit (Zymo Research, California, USA). Briefly, 5 μl (2 μg) of the NotI digested and linearised expression vector was mixed with 50 μl competent cells and 500 μl of EZ3 solution from the kit and mixed thoroughly. A negative control transformation included competent cells without any DNA of the genetic construct. The mixtures were incubated at 28° C. for two hours and then 100 μl spread on a SD-Ura plate or other selective plates as appropriate e.g. antibiotic containing medium. The plates were incubated for two days at 28° C. When the recipient strain was an auxotroph lacking a functional URA3 gene, only transformants having received the vector with the URA gene grew on these plates. Many colonies were obtained in the Y. lipolytica transformations. Ura⁺ or antibiotic resistant colonies were picked from the selection plates and confirmed as transformed by colony PCR for the introduced genetic construct and the phenotype corresponding to intended genetic modification.

### Gene Expression Analysis

Expression of transgenes was analysed using a DNase RQ 1 kit (Promega Catalog No. M6101) and a Qiagen column (Qiagen RNAse-free DNAse) to purify RNA from the cells, and oligo dT primer (200-500 ng), dNTPs (10 mM), Superscript III reverse transcriptase and 0.1 M DTT for reverse transcription using standard methods.

### Example 2. Extraction of Lipids from Microbes

Lipid extraction from yeasts such as S. cerevisiae and Y. lipolytica is made more difficult by the rigid cell wall of these organisms. Various methods have been described in the literature for cell disruption and lipid extraction from yeasts, including mechanical, enzymatic, chemical, osmotic shock and microwave methods of cell disruption (Hein and Hayen, 2012). Chisti and Moo-Young (1986) reviewed mechanical, chemical and enzymatic methods of microbial cell disruption as well as cell lysis by osmotic shock. Hegel et al. (2011) described lipid extraction from yeast using supercritical carbon dioxide. Peter et al. (2017) reported cell disruption and homogenization of Schizosaccharomyces pombe cells in a water/methanol solvent mixture using zirconium oxide beads, a bullet blender and a water bath sonicator.

A simple, high throughput and small-scale lipid extraction method was desirable to perform the studies described in the following Examples. The inventors therefore tested several methods and variations for the extraction of lipids from S. cerevisiae and Y. lipolytica, in particular tested some methods for cell wall disruption and homogenisation of the cellular material with organic solvents to extract the lipid and testing the efficiency of extraction.

### Experiment 1—Extraction of Lipids from S. cerevisiae

In a first experiment aiming to test lipid extraction efficiency from yeast cells using ultrasonication for cell disruption in the presence of either a KCl solution or methanol, S. cerevisiae strain INVSc1 was grown in 5 ml of YPD medium for 3 days. The cells were harvested by centrifugation, washed with water and freeze dried as described in Example 1. Identical dried cell pellets of about 25 mg in 2 ml tubes were treated in four ways:
1A. Homogenization in KCl solution, lipid extraction using chloroform/methanol.
1B. Homogenization in KCl solution, sonication for 5 min, lipid extraction using chloroform/methanol.
2A. Homogenization in methanol, lipid extraction using chloroform/methanol/KCl.
2B. Homogenization in methanol, 5 min sonication, lipid extraction using chloroform/methanol/KCl.

In method 1Δ, 0.3 ml 1M KCl was added to the tube and the cells were disrupted using zirconium beads (Catalog No. ZROB05, Next Advance, Inc., USA) and a Bullet Blender Blue (Next Advance, Inc. USA) at speed 8 for 3 min, followed by addition of 0.4 ml methanol and 0.8 ml chloroform. The mixture was shaken for 5 min and centrifuged for 5 min at 10,000 g. The lower phase containing lipid was transferred to a glass vial. For method 1B, the only difference was an additional step of ultrasonication of the mixture using a water bath sonicator (Bransonic M2800H-E, Branson Ultrasonic Corporation, USA) for 5 min after addition of the methanol and before addition of chloroform. In method 2Δ, 0.3 ml methanol was added to the tube containing yeast cells and zirconium beads and homogenized in the Bullet Blender, followed by addition of 0.3 ml 1 M KCl, 0.1 ml methanol and 0.8 ml chloroform. The mixture was shaken, centrifuged and lower phase was collected as before. Method 2B was the same as 2A except that an ultrasonication was carried out after cell disruption in the Bullet Blender.

For each sample, the solvent was evaporated from the lipid sample under a flow of nitrogen gas and the extracted lipid dissolved in a measured volume of chloroform. To measure the amount of extracted lipid in each sample, a measured aliquot of the lipid in chloroform was transferred to a GC vial having a PTTE-lined screw cap. After evaporation of the chloroform under nitrogen gas, a known amount of triheptadecanoin (Nu-Chek Prep, Inc., Catalog No. T-155, Waterville, MN, USA) was added to the vial. The fatty acids in each lipid sample were converted to FAME and measured by GC as described in Example 1. The peak areas were integrated and compared to the known amount of heptadecanoin to calculate the amount of fatty acids by weight in the extracted lipids.

As a control to measure the total amount of lipid present in the cells prior to extraction, all of the lipids in duplicate, identical cell pellets were converted to FAME by direct methylation with methanolic-HCl together with triheptadecanoin and analysed by GC. The average of the two controls provided the total fatty acid content, taken as 100% of the cellular fatty acid content. Comparison of the amount of total fatty acid content in the extracted lipids and the cellular fatty acid content provided the extraction efficiency for the four tested methods.

Table 4 provides the data from this experiment. Among the four methods tested in this experiment, method 2B provided the most efficient lipid extraction from the freeze-dried S. cerevisiae cells, yielding 62.4% of the total cellular fatty acid content. Method 2B included cell disruption in methanol with the zirconium beads and bullet blender and then ultrasonication. On the other hand, method 1B yielded 26.2% lipid extraction efficiency, with homogenisation in KCl solution with ultrasonication for cell disruption. Methods 1A and 2A did not use ultrasonication and yielded lower lipid extraction efficiency.

### Experiment 2

Another experiment was carried out to estimate lipid extraction efficiency with a larger cell sample and to compare with a method where cells were disrupted in a mixture of chloroform/methanol (2/1, v/v). Dry cell pellets of about 47 mg and 0.5 g zirconium beads were transferred to 2 ml Eppendorf tubes. In method 3Δ, the efficiency of lipid extraction using ultrasonication was tested. For this, 0.4 ml methanol was added to the tube and the mixture was treated with ultrasonication in a water bath at 40° C. for 10 min. Then, 0.3 ml 1 M KCl and 0.8 ml chloroform were added to the tube and the mixture vortexed for 5 min, followed by centrifugation for 5 min at 10,000 g. The lower phase was collected in a glass vial. Lipid was extracted a second time from the upper phase by adding 0.8 ml chloroform and vortexing the mixture for 5 min, followed by centrifugation and collection of the lower phase which was combined with the first extract in the glass vial. Method 3B used both the zirconium beads and Bullet Blender at speed 8 for 5 min and ultrasonication for 10 min for cell disruption in 0.4 ml methanol, otherwise was the same as method 3B. Method 4 tested cell disruption in a mixture of chloroform/methanol (2/1, v/v) rather than methanol. Extracted lipids were treated and quantitated as for Experiment 1. As in Experiment 1, direct methylation of fatty acids in the cell samples provided the total fatty acid content, taken as 100%.

The data are presented in Table 4. When cells were disrupted in methanol with sonication (Method 3A), 27% of the total lipid content lipid was extracted from the cells. Cellular disruption in methanol using the bullet blender and additionally by sonication yielded 46.4% of the total lipid. On the other hand, cellular disruption in the mixture of chloroform/methanol (2/1, v/v), followed by sonication yielded the lowest level of extracted lipid from S. cerevisiae.

### Experiment 3

A third experiment compared lipid extraction efficiencies from S. cerevisiae cells using glass beads, zirconium beads or metal balls, and using the bead beater or vortexing for the homogenisation of the cells in methanol. Cells from 10 ml cultures were obtained as for the previous experiments and identical cell pellets were treated. Glass beads, zirconium beads or metal balls were added to the tubes and either vortexed or mixed using the bullet blender, as follows.

Method 5: 0.3 ml methanol, 0.5 g glass beads (Catalog No. G8772, Sigma) and two 1 mm metal balls were added to a tube containing the cell pellet, vortexed for 10 min using the Vibramax.

Method 6: 0.3 ml methanol, 0.5 g zirconium beads (Catalog No. ZROB05, Next Advance, Inc., USA) and two 1 mm metal balls were added to the second tube containing the cells and vortexed for 10 min.

Method 7: 0.3 ml methanol and 0.5 g zirconium beads were added to the third tube containing the cell pellet and vortexed for 10 min.

Method 8: 0.3 ml methanol and 0.5 g zirconium beads were added to the fourth tube containing the cell pellet and shaken in a TissueLyser II (Qiagen Inc., Germantown, MD, USA) for 3 min at 25 rpm/sec.

After the homogenisation, 0.4 ml of 1 M KCl, 0.1 ml methanol and 0.8 ml chloroform were added to each tube and the mixtures vortexed for another 5 min. The mixtures were centrifuged at 10,000 g for 5 min and the lower, chloroform phase was transferred to a glass vial. The extracted lipid samples were dried and the fatty acids converted to FAME and quantitated by GC as in the previous experiments.

During the lipid extraction processes, cell debris accumulated at the interphase after the centrifugation of the mixtures. To measure the lipid remaining in the cell debris and so determine the total lipid content, the cell debris was dried in a freeze dryer. A known amount of triheptadecanoin was added and the fatty acids converted to FAME using 0.7 ml methanolic-HCl with incubation at 80° C. for 2 h. FAME were quantitated by GC as before.

The data are presented in Table 4. The most efficient extraction was with method 8, using zirconium beads with the bead beater, extracting 66.5% of the total fatty acid content. Methods 5-7 yielded less extracted lipid than method 8 (Table 4). The efficiency of lipid extraction using the bead beater (method 8) was similar to methods 2B and 3B which involved cell disruption using the bullet blender and sonication. The fatty acid composition of the lipid remaining in the cell debris after the first extraction was the same as for the extracted lipid.

TABLE 4
Lipid extraction from S. cerevisiae using different methods of cell disruption.

| | | | | | | | | | | | | |
|---|---|---|---|---|---|---|---|---|---|---|---|---|
| | | | | | | | | | | | Lipid | % of |
| | | | | | | C16:1 | C16:1 | | C18:1 | C18:1 | %/ | total |
| C10:0 | C12:0 | C14:0 | C14:1 | C15:0 | C16:0 | Δ7 | Δ9 | C18:0 | Δ9 | Δ11 | DCW | lipid |

Experiment 1

| | | | | | | | | | | | | | |
|---|---|---|---|---|---|---|---|---|---|---|---|---|---|
| 1A | 0.9 | 0.8 | 0.7 | 0.3 | 0.2 | 10.5 | 0.6 | 55.1 | 3.7 | 24.5 | 2.8 | 0.7 | 18.8 |
| 1B | 1.0 | 0.9 | 0.8 | 0.3 | 0.2 | 11.0 | 0.6 | 54.1 | 3.9 | 24.5 | 2.7 | 1.0 | 26.2 |
| 2A | 1.1 | 0.9 | 0.9 | 0.3 | 0.2 | 10.2 | 0.7 | 55.0 | 3.6 | 24.4 | 2.6 | 1.9 | 50.3 |
| 2B | 1.1 | 1.0 | 0.9 | 0.4 | 0.2 | 10.4 | 0.7 | 54.4 | 3.7 | 24.6 | 2.6 | 2.3 | 62.4 |
| total | 0.8 | 0.8 | 0.7 | 0.2 | 0.2 | 11.7 | 0.8 | 52.2 | 4.2 | 25.0 | 3.3 | 3.8 | 100% |

Experiment 2

| | | | | | | | | | | | | |
|---|---|---|---|---|---|---|---|---|---|---|---|---|
| 3A | 1.3 | 0.9 | 0.9 | 0.4 | 0.2 | 10.0 | 54.0 | 3.6 | 26.1 | 2.7 | 1.1 | 27.0 |
| 3B | 1.3 | 0.9 | 0.9 | 0.4 | 0.2 | 10.0 | 54.0 | 3.5 | 26.4 | 2.6 | 1.9 | 46.4 |
| 4 | 1.6 | 1.0 | 0.9 | 0.4 | 0.2 | 9.3 | 54.6 | 3.4 | 25.9 | 2.7 | 1.0 | 24.1 |
| total 1 | 1.1 | 0.8 | 0.8 | 0.3 | 0.2 | 11.8 | 50.7 | 4.3 | 27.5 | 2.5 | 4.1 | 100% |
| total 2 | 1.0 | 0.8 | 0.8 | 0.3 | 0.2 | 11.8 | 48.7 | 4.6 | 29.3 | 2.4 | 4.2 | 100% |

Experiment 3

| | | | | | | | | | | | | | |
|---|---|---|---|---|---|---|---|---|---|---|---|---|---|
| 5 | 1.6 | 0.9 | 1.1 | 0.4 | 0.4 | 12.7 | 0.8 | 56.3 | 4.1 | 19.9 | 1.8 | 304 | 33.5 |
| 6 | 1.6 | 0.9 | 1.1 | 0.4 | 0.3 | 12.7 | 0.7 | 55.4 | 4.2 | 20.7 | 1.9 | 429 | 50.5 |
| 7 | 1.5 | 0.9 | 1.1 | 0.4 | 0.4 | 12.8 | 0.7 | 55.7 | 4.1 | 20.5 | 1.9 | 376 | 43.9 |
| 8 | 1.4 | 0.9 | 1.1 | 0.4 | 0.3 | 12.9 | 0.7 | 54.3 | 4.4 | 21.5 | 1.9 | 480 | 66.5 |
| Cell debris 1 | 1.1 | 0.9 | 1.0 | 0.4 | 0.4 | 14.8 | 0.7 | 51.4 | 5.2 | 22.3 | 1.8 | 603 | 66.5 |
| Cell debris 2 | 1.0 | 0.9 | 1.0 | 0.4 | 0.4 | 14.9 | 0.7 | 50.5 | 5.4 | 23.0 | 1.8 | 420 | 49.5 |
| Cell debris 3 | 1.0 | 0.9 | 1.0 | 0.4 | 0.4 | 14.6 | 0.7 | 51.0 | 5.2 | 23.1 | 1.8 | 481 | 56.1 |
| Cell debris 4 | 1.1 | 0.9 | 1.0 | 0.4 | 0.4 | 15.7 | 0.7 | 49.7 | 5.7 | 22.8 | 1.7 | 242 | 33.5 |

### Experiment 4. Extraction of Lipids from Y. lipolytica

For many analyses in Y. lipolytica where the primary purpose was to determine the fatty acid composition of the cells and maximal extraction efficiency was not needed, the inventors decided to routinely use a simpler method that was more suited to high throughput of samples, yet provided sufficient extracted lipid. This conclusion was in view of the observation made in the experiments described above that the fatty acid composition of the extracted lipid was the same as the composition of the residual lipid remaining in the cell debris (Table 4), and so was representative of the total fatty acid content of the cells. In brief, this method, described in Example 1, homogenised dried cell pellets and disrupted the cells in chloroform/methanol (2/1, v/v) solution with zirconium beads using the bullet blender, followed by sonication in the waterbath sonicator and mixing for 20 min. After addition of KCl solution, the mixture was vortexed for 10 min and centrifuged to separate phases. The lower phase was collected. Lipid remaining in the upper phase was extracted using another volume of chloroform and the extracts combined and dried down.

Another experiment was carried out to determine whether Y. lipolytica cells could be heat treated at 105° C. for 5 min immediately after harvesting the culture by centrifugation, to kill the cells and likely inactivate any lipases in or from the cells. Additionally, aliquots of the cells were either freeze dried or not freeze dried prior to lipid extraction. When lipid extracts from wild-type W29 cells were analysed by TLC, it was observed that the heat-treated cells which were not dried yielded less free fatty acids (FFA) than the cells that had been dried, suggesting that the heat treatment was effective. The heat treatment itself did not affect either the lipid content or fatty acid content of the cells. Extraction of lipids from cells which had not been dried down was at least as efficient as from cells which had been freeze dried.

### Example 3. Content and Composition of Lipids from Microbes

The present inventors wanted to determine the content and composition of the total lipid content from various microbes, including the triacylglycerol (TAG) and polar lipids as a baseline before genetic modifications of the microbes to modify the lipid content and composition.

### Growth of Microbes and Extraction of Lipids

In order to determine the amount and fatty acid composition of total lipids including polar lipids and TAG in microbial cells during their growth cycle, five widely used strains of three different species were selected. These were E. coli strains DH5a and BL21, the oleaginous wild-type Y. lipolytica strain W29, and S. cerevisiae strains INVSc1 and D5Δ. These species were chosen due to the availability of genetic tools and processes for genetic engineering as well as the depth of knowledge about lipid synthesis and metabolism in these species. Strain D5A was selected as an oleaginous strain of S. cerevisiae (He et al., 2018). These microbes were cultured for up to 7 days, with removal and analysis of samples at different time points. Inoculum cultures were prepared by growing cells overnight in LB medium for E. coli or YPD or SD+Ura media for the yeasts. Samples of these cultures were diluted into 200 ml of the same growth medium in 1 L bottles to provide an initial OD600 of 0.1. The mouth of each bottle was covered by micropore tape and the cultures were shaken for aeration. The E. coli cells were incubated in a shaker at 37° C. at 250 rpm. Yeast cells were grown in the YPD medium containing 2% glucose as carbon source and incubated at 28° C. with shaking at 200 rpm. Samples of 10 ml were removed from each culture at 18 h, 24 h, 2 d, 3 d, 4 d, 5 d, 6 d and 7 d time points. Cells were harvested from the cultures by centrifugation at 3,400 g for 10 min and washed twice with 3 ml each time of de-ionised water and once with 1.5 ml de-ionised water. The cells were transferred to pre-weighed 2 ml tubes and freeze dried for 24 h. The tubes were then weighed again and the dry cell weights were calculated prior to lipid extraction.

Total cellular lipid was extracted as described in Example 1, using 0.6 ml chloroform/methanol (2/1, v/v) as the extraction solvent in the presence zirconium beads using a bullet blender, followed by sonication in a water bath at 40° C. After mixing the homogenate with 0.3 ml 0.1 M KCl for 10 min, the mixture was centrifuged at 10,000 g for 5 min. The lower phase containing lipid was transferred to a glass vial. Remaining lipid was extracted from the upper phase containing the cell debris with 0.6 ml chloroform for 20 min, centrifugation and the collection of the lower phase as before. The solvent was evaporated from the combined lower phases under a flow of nitrogen gas and the extracted lipid was resuspended in a measured volume of chloroform. Aliquots of lipid extracted from 20 mg dry cell weight were fractionated on a TLC plate using a solvent mixture of hexane/diethylether/acetic acid (70/30/1, v/v/v) to separate TAG and polar lipids, as described in Example 1. The fatty acid composition of the lipid from the TAG and polar lipid spots were determined by GC of FAME produced from the lipids, again as described in Example 1.

Initial Experiment with S. cerevisiae

In an initial experiment, lipid was extracted from cultured cells of S. cerevisiae strain INVSc1 after growth for 1, 2, 3 or 4 days in YPD and SD+Ura media. The data are shown in Table 5, including the extracted lipid yield as a percentage of dry cell weight (dcw). The efficiency of recovery of the TAG and polar lipids in the TLC fractionation was not determined. It was noted that the amount of TAG produced by the INVSc1 cells was low when cultured in YPD medium, while higher in SD+Ura medium. Polar lipid yields were between 0.63% and 1.15% on a dry cell weight basis, but the method was not maximised for efficient extraction. For the fatty acid composition, both fractions contained 47-67% of C16:1Δ9 as the fatty acid present in the greatest amount. Oleic acid (C18:1Δ9) and palmitic acid (C16:0) were the other main fatty acids present, as was a low level of stearic acid (C18:0), while linoleic acid (LA, C18:2Δ^{9,12}) was not present. These data were consistent with published reports (e.g. Itoh and Kaneko, 1974; Stukey et al., 1989; Kamisaka et al., 2015) that reported the presence of 40-55% of C16:1, 30-35% of C18:1Δ9, and lesser amounts of C16:0 and C18:0. These four fatty acids make up almost all of the fatty acid content in many wild-type S. cerevisiae strains. Wild-type strains such as INVSc1 contain only one fatty acid desaturase, a Δ9-desaturase encoded by the OLE1 gene, which produces the monounsaturated palmitoleic and oleic acids (Stukey et al., 1989).

Like S. cerevisiae, the wild-type fission yeast S. pombe is unable to synthesize LA and other polyunsaturated fatty acids (Ratledge and Evans 1989; Holic et al., 2012). In contrast, other wild-type yeasts such as S. kluyveri and K. lactis have Δ12- and Δ15-desaturases and can produce LA and ALA.

Fatty Acid Composition in E. coli, Y. lipolytica and S. cerevisiae after Growth for Up to 7 Days

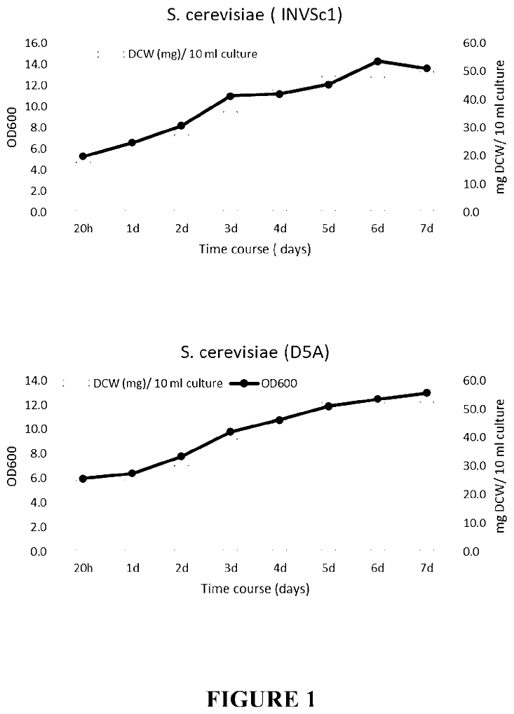

An experiment was carried out with E. coli, S. cerevisiae and Y. lipolytica, sampling the cultures daily up to 7 days. The growth curves for two S. cerevisiae strains are provided in FIG. 1, showing OD600 and the dry cell weight over the 7 days of culturing. The amount of lipid and fatty acid compositions were determined for both the polar lipid and TAG fractions for each strain at each time point. The data are presented in Table 6 for two E. coli strains, Table 7 for Y. lipolytica strain W29 and Table 8 for S. cerevisiae strains INVSc1 and D5Δ. The identity of the fatty acid C15:0 (pentadecanoic acid) was confirmed by GC-MS. The fatty acid composition of the polar lipid of E. coli strain BL21 was similar to that reported by Kanemasa et al. (1967) and Marr and Ingraham (1962) for other, wild-type E. coli strains. As for many other bacteria, E. coli polar lipids contain four types of fatty acids: straight chain saturated fatty acids including C12:0, C14:0, C15:0 and C16:0, straight chain monounsaturated fatty acids including C16:1Δ9 (cis-palmitoleic acid) and C18:1Δ11 (cis-vaccenic acid), branched chain fatty acids, and cyclopropane fatty acids including C17:0c* (cis-9,10-methylene hexadecenoic acid) and C19:0c*(cis-11,12-methylene octadecenoic acid) (Hildebrand and Law, 1964). The presence of C16:0, C16:1, C18:0 and C18:1Δ11 was reported in E. coli strain BL21 by Oldham et al. (2001). The unsaturated fatty acids found in wild-type E. coli are all monoenes of the cis conformation, but do not include oleic acid (Cronan and Vagelos, 1972). The four fatty acid types were all observed in the extracted lipid from BL21, which had about 31-36% C18:1Δ11 and about 7-10% C16:1Δ9, as well as 30-35% of the saturated fatty acid C16:0 (palmitic acid), 10-20% of the cyclopropane fatty acid C17:0c* and 1-5% of C19:0c*. These latter two fatty acids are distinctive for bacterial lipids, being rarely found in animal fats or yeast lipids. They are produced from the corresponding monoenes C16:1Δ9 and C18:1Δ11 through the activity of a cyclopropane fatty acid synthase (CPFAS). Another difference observed with animal fats was that polyunsaturated fatty acids such as LA were not present in wild-type E. coli lipids, and this was observed for BL21 and DH5a. Additionally, oleic acid (C18:1Δ9) was not observed in the E. coli polar lipid but is present at substantial levels in animal and plant lipids.

Strain DH5a exhibited a significantly different fatty acid composition to BL21 in terms of the amounts of some fatty acids in its polar lipid, having considerably less C18:1Δ11 at about 3-8% and less C16:1Δ9, but more C16:0 and considerably more C15:0 and cyclopropane fatty acids. In DH5a, almost half of the total fatty acid content was palmitic acid, which was reported to be located almost exclusively at the sn-1 position of the phospholipid (Cronan and Vagelos, 1972). Hildebrand and Law (1964) reported the presence of cyclopropane fatty acids in E. coli, and they were also observed here in DH5a. As shown in Table 9, pentadecanoic acid, nonadecanoic acid (C19:0) and the cyclopropane fatty acids were observed in the polar lipid fraction. The decrease in DH5a relative to BL21 in the levels of C16:1, and C18:1Δ11 was accompanied by increased amounts of C14:0, C15:0, C16:0 and the cyclopropane fatty acids. Both E. coli strains had less than 2% stearic acid (C18:0) in their lipids. The highest amount of polar lipid was observed at day 2 of culturing, at about 2.7% DCW.

The fatty acid composition of Y. lipolytica (Table 7) was quite different to that of E. co/i and S. cerevisiae. A wider range of fatty acids was observed in Y. lipolytica lipid, including, for example, polyunsaturated fatty acids such as LA and the longer chain, saturated fatty acids having 20, 22 or 24 carbons, C20:0, C22:0 and C24:0 (VLC-SFA) which were all present in the TAG fraction. C24:0 was present in the TAG fraction at a level of between 3% and 9% at most timepoints. The peak for this fatty acid in the GC chromatogram was at the same position as a C24:0 standard, and GC-MS also confirmed the identity of the peak as C24:0. C24:0 was generally present and C20:0 and C22:0 were either absent or present at low levels of the total fatty acid content of the polar lipid fraction (<0.5%) on a weight basis. Although Y. lipolytica is an oleaginous microbe, the growth conditions in this experiment using rich YPD medium did not favour high level TAG production, so producing less than about 1% TAG on a dry cell weight basis. TAG continued accumulating at that low level during the 7-day period. The highest level of polar lipid was observed at day 2 of the culture. Palmitic, palmitoleic, oleic and linoleic acids were the major fatty acids in Y. lipolytica. The polar lipid also contained short, medium and long-chain saturated and monounsaturated fatty acids at low levels, together with odd chain fatty acids such as pentadecanoic acid and heptadecenoic acid (Table 7). The identity of the peak for pentadecanoic acid was also confirmed by GC-MS. The fatty acid composition was similar to that reported by Carsanba et al., (2020). However, the present inventors were not aware of any previous reports of the presence of C24:0 in TAG in Y. lipolytica at the levels observed here.

The polar lipid and TAG fractions of Y. lipolytica showed significantly different amounts of some fatty acids. In general, the polar lipid contained higher levels of LA and palmitoleic acid (C16:1) than TAG, while the TAG was richer in palmitic, stearic acid and lignoceric acids (C24:0). In particular, the TAG had much greater levels of the saturated fatty acid stearic acid at about 4-12% compared to less than 1% in the polar lipids, as well as greater amounts of the saturated C20, C22 and C24 fatty acids. The Y. lipolytica polar lipid was easily distinguishable from the E. coli lipid, for example the former had C18:1Δ9 (oleic acid) rather than C18:1Δ11 (vaccenic acid) as the predominant monounsaturated fatty acid. As noted above, E. coli lipid lacked oleic acid.

The polar lipid and TAG fractions from S. cerevisiae strains INVSc1 and D5A contained mostly four fatty acids, the monounsaturated fatty acid palmitoleic acid (C16:1Δ9) and oleic acid (C18:1Δ9) and the saturated fatty acids palmitic acid (C16:0) and stearic acid (C18:0). These data were consistent with published reports (He et al., 2018). The polar lipid fractions were slightly higher in the saturated fatty acids and lower in the monounsaturated fatty acids relative to the TAG fractions.

Screening of Other Y. lipolytica Strains

Five other Y. lipolytica strains from a CSIRO microbial collection were screened for lipid production and fatty acid composition. The strains were grown in SD-Ura medium for 48 h as starter cultures. Samples of the starter cultures were centrifuged, the cell pellets resuspended in the DM-Gluc medium and used to inoculate cultures in DM-Gluc medium containing 8% (w/v) glucose as carbon source and 4 g/l di-ammonium hydrogen orthophosphate as the main nitrogen source (Example 1). Cells were harvested after 48 h, lipid extracted and fractionated by TLC. The TAG and polar lipid fractions were analysed by GC of FAME as described in Example 1. The data for four of the strains are shown in Table 9. The strains all produced TAG with similar fatty acid profiles to the wild-type strain W29. The main fatty acids in the TAG fraction produced under these culture conditions were oleic acid (30-45%), palmitic acid (10-17%), stearic acid (12-24%) and linoleic acid (4-7%) on a weight basis. Notably, the accumulated TAG comprised 2.6-5.5% C24:0 and C20:0 and C22:0 were also present at about 1-2% each.

TABLE 5
Fatty acid composition of TAG and polar lipid fractions of S. cerevisiae strain INVSc1 cells during culturing for 1 to 4 days.

| | | | | | | | | | | | | | | |
|---|---|---|---|---|---|---|---|---|---|---|---|---|---|---|
| Lipid | | | | | | | | | | | | | | |
| fraction/days | | | | C14:1 | | | | C16:1 | C16:1 | | C18:1 | C18:1 | C18:2 | % of |
| of culture | C10:0 | C12:0 | C14:0 | Δ9 | C15:0 | C16:0 | C16:1t | Δ7 | Δ9 | C18:0 | Δ9 | Δ11 | Δ9, 12 | dew |

YPD medium

| | | | | | | | | | | | | | | |
|---|---|---|---|---|---|---|---|---|---|---|---|---|---|---|
| TAG-1 day | 1.0 | 1.3 | 1.4 | 0.4 | 0.0 | 10.4 | 0.3 | 0.0 | 57.2 | 3.6 | 21.1 | 3.2 | 0.0 | 0.26 |
| TAG-2 days | 1.0 | 1.2 | 0.7 | 0.0 | 0.0 | 7.2 | 0.0 | 0.0 | 67.7 | 3.1 | 19.1 | 0.0 | 0.0 | 0.28 |
| TAG-3 days | 0.6 | 0.7 | 0.9 | 0.0 | 0.0 | 9.4 | 0.0 | 0.0 | 60.4 | 4.0 | 24.0 | 0.0 | 0.0 | 0.52 |
| TAG-4 days | 0.2 | 0.5 | 0.9 | 0.0 | 0.0 | 11.9 | 0.0 | 0.0 | 57.4 | 3.8 | 25.3 | 0.0 | 0.0 | 0.66 |
| Polar-1 day | 0.9 | 0.6 | 1.2 | 0.3 | 0.3 | 20.0 | 0.7 | 0.0 | 51.7 | 2.4 | 20.2 | 1.8 | 0.0 | 0.75 |
| Polar-2 days | 0.7 | 0.3 | 0.8 | 0.2 | 0.4 | 13.7 | 0.7 | 0.0 | 54.1 | 3.1 | 23.1 | 2.7 | 0.0 | 0.63 |
| Polar-3 days | 0.3 | 0.2 | 0.7 | 0.2 | 0.3 | 11.8 | 0.7 | 0.0 | 53.2 | 3.0 | 27.1 | 2.6 | 0.0 | 1.08 |
| Polar-4 days | 0.1 | 0.1 | 0.6 | 0.1 | 0.3 | 11.5 | 0.7 | 0.0 | 51.8 | 2.8 | 29.4 | 2.6 | 0.0 | 1.15 |

SD + Ura medium

| | | | | | | | | | | | | | | |
|---|---|---|---|---|---|---|---|---|---|---|---|---|---|---|
| TAG-1 day | 0.0 | 0.5 | 0.7 | 0.2 | 0.0 | 8.9 | 0.4 | 0.0 | 56.5 | 4.8 | 25.0 | 3.0 | 0.0 | 0.31 |
| TAG-2 days | 0.0 | 0.2 | 0.5 | 0.1 | 0.3 | 13.9 | 0.4 | 0.0 | 53.8 | 3.6 | 24.4 | 2.8 | 0.0 | 0.85 |
| TAG-3 days | 0.0 | 0.2 | 0.5 | 0.1 | 0.3 | 17.2 | 0.4 | 0.0 | 51.5 | 4.5 | 22.7 | 2.7 | 0.0 | 1.21 |
| TAG-4 days | 0.0 | 0.2 | 0.5 | 0.0 | 0.3 | 19.3 | 0.4 | 0.0 | 48.1 | 5.8 | 22.8 | 2.8 | 0.0 | 1.69 |
| Polar-1 day | 0.3 | 0.3 | 0.7 | 0.2 | 0.2 | 13.4 | 0.7 | 0.0 | 51.5 | 4.1 | 26.3 | 2.2 | 0.0 | 0.94 |
| Polar-2 days | 0.2 | 0.1 | 0.4 | 0.1 | 0.3 | 12.9 | 0.7 | 0.0 | 50.0 | 3.7 | 28.9 | 2.8 | 0.0 | 0.65 |
| Polar-3 days | 0.1 | 0.1 | 0.3 | 0.1 | 0.3 | 12.7 | 0.7 | 0.0 | 48.5 | 3.4 | 30.7 | 3.1 | 0.0 | 0.89 |
| Polar-4 days | 0.3 | 0.1 | 0.3 | 0.0 | 0.3 | 14.4 | 0.8 | 0.0 | 47.3 | 4.1 | 29.5 | 3.0 | 0.0 | 0.94 |

TABLE 6
Fatty acid composition and amount of polar lipid in E. coli during culturing in LB medium.

| | | | | | | | | | | |
|---|---|---|---|---|---|---|---|---|---|---|
| | | | | C16:1 | | C18:1 | | | | % of |
| C12:0 | C14:0 | C15:0 | C16:0 | Δ9 | C18:0 | Δ11 | C19:0 | C17:0c* | C19:0c* | dcw |

E. coli strain DH5α

| | | | | | | | | | | | |
|---|---|---|---|---|---|---|---|---|---|---|---|
| 20 h | 0.6 | 5.6 | 3.8 | 47.9 | 6.4 | 0.8 | 7.8 | 4.6 | 18.3 | 4.1 | 1.4 |
| 1 d | 0.9 | 6.1 | 4.3 | 46.9 | 7.3 | 0.7 | 7.5 | 4.4 | 17.7 | 4.1 | 2.0 |
| 2 d | 0.6 | 5.8 | 8.1 | 46.4 | 2.6 | 0.3 | 4.6 | 6.3 | 20.3 | 4.9 | 2.7 |
| 3 d | 0.5 | 5.4 | 9.0 | 45.8 | 2.1 | 1.2 | 4.2 | 6.4 | 20.6 | 4.9 | 1.9 |
| 4 d | 0.4 | 5.0 | 10.6 | 44.3 | 1.6 | 1.2 | 3.7 | 6.9 | 21.3 | 5.0 | 2.4 |
| 5 d | 0.3 | 4.6 | 12.0 | 44.7 | 1.6 | 1.6 | 3.6 | 9.2 | 18.2 | 4.2 | 1.1 |
| 6 d | 0.3 | 4.1 | 13.2 | 41.9 | 2.0 | 1.5 | 3.2 | 8.9 | 20.2 | 4.6 | 1.0 |
| 7 d | 0.5 | 4.5 | 14.8 | 39.4 | 4.1 | 0.2 | 3.3 | 8.7 | 19.9 | 4.0 | 1.1 |

E. coli strain BL21

| | | | | | | | | | | | |
|---|---|---|---|---|---|---|---|---|---|---|---|
| 20 h | 0.5 | 3.7 | 0.2 | 35.4 | 8.9 | 1.0 | 31.0 | 3.0 | 11.7 | 4.5 | 1.9 |
| 1 d | 2.0 | 4.5 | 0.2 | 33.3 | 9.3 | 0.9 | 31.5 | 2.8 | 11.4 | 4.0 | 1.8 |
| 2 d | 0.8 | 2.4 | 0.4 | 30.3 | 8.3 | 1.0 | 36.2 | 4.4 | 13.8 | 2.3 | 2.0 |
| 3 d | 0.5 | 1.7 | 0.5 | 30.9 | 7.4 | 1.2 | 35.0 | 4.9 | 15.7 | 2.1 | 1.3 |
| 4 d | 0.5 | 1.6 | 0.5 | 30.7 | 7.0 | 1.2 | 35.2 | 5.4 | 15.9 | 2.0 | 1.5 |
| 5 d | 1.0 | 2.1 | 0.4 | 30.6 | 6.9 | 1.4 | 36.5 | 6.5 | 12.8 | 1.7 | 0.5 |
| 6 d | 1.4 | 2.4 | 0.4 | 30.1 | 7.2 | 1.3 | 36.2 | 5.9 | 13.3 | 1.8 | 0.4 |
| 7 d | 1.3 | 2.3 | 0.4 | 29.7 | 9.7 | 1.0 | 35.7 | 6.0 | 12.1 | 1.6 | 0.9 |

*Cyclopropane fatty acids.

TABLE 7
Fatty acid composition of TAG and polar lipid fractions
from Y. lipolytica during culturing in YPD medium.

| | | | | | | | | | |
|---|---|---|---|---|---|---|---|---|---|
| | | | | | C16:1 | C16:1 | | | C18:1 |
| C12:0 | C12:1 | C14:0 | C15:0 | C16:0 | Δ7 | Δ9 | C17:1 | C18:0 | Δ9 |

Polar lipid

| | | | | | | | | | | |
|---|---|---|---|---|---|---|---|---|---|---|
| 20 h | 0.1 | 0.1 | 0.3 | 0.7 | 11.6 | 0.9 | 12.1 | 1.8 | 0.7 | 55.8 |
| 1 d | 0.1 | 0.0 | 0.2 | 0.5 | 9.8 | 0.9 | 12.5 | 1.8 | 0.5 | 57.0 |
| 2 d | 0.2 | 0.0 | 0.4 | 0.5 | 10.1 | 1.4 | 13.9 | 2.7 | 0.4 | 55.0 |
| 3 d | 0.1 | 0.1 | 0.4 | 0.3 | 10.8 | 2.2 | 12.7 | 3.0 | 0.5 | 53.5 |
| 4 d | 0.2 | 0.3 | 0.5 | 0.2 | 11.5 | 2.4 | 12.1 | 2.7 | 0.5 | 53.2 |
| 5 d | 0.2 | 0.2 | 0.8 | 0.2 | 13.6 | 3.2 | 13.6 | 2.1 | 0.6 | 49.6 |
| 6 d | 0.2 | 0.0 | 0.5 | 0.2 | 11.2 | 3.2 | 13.7 | 1.9 | 0.4 | 48.4 |
| 7 d | 0.1 | 0.0 | 0.5 | 0.2 | 9.7 | 2.6 | 13.6 | 1.6 | 0.3 | 47.5 |

Triacylglycerols

| | | | | | | | | | | |
|---|---|---|---|---|---|---|---|---|---|---|
| 20 h | 0.2 | 0.4 | 0.4 | 0.5 | 10.3 | 0.7 | 10.7 | 1.6 | 5.6 | 51.1 |
| 1 d | 0.0 | 0.2 | 0.0 | 0.6 | 15.2 | 0.6 | 8.6 | 1.2 | 12.4 | 41. |
| 2 d | 0.0 | 0.0 | 0.2 | 0.5 | 15.4 | 0.8 | 9.5 | 1.6 | 11.0 | 43.3 |
| 3 d | 0.0 | 1.1 | 0.1 | 0.4 | 15.8 | 1.2 | 8.1 | 1.7 | 11.8 | 41.5 |
| 4 d | 0.0 | 0.4 | 0.1 | 0.2 | 13.8 | 1.4 | 8.7 | 1.0 | 10.2 | 44.9 |
| 5 d | 0.1 | 1.0 | 0.1 | 0.2 | 10.1 | 1.8 | 11.2 | 1.8 | 6.5 | 52.1 |
| 6 d | 0.1 | 0.8 | 0.1 | 0.2 | 7.8 | 1.2 | 11.8 | 1.5 | 5.2 | 55.4 |
| 7 d | 0.0 | 0.0 | 0.1 | 0.1 | 8.6 | 0.9 | 12.2 | 1.0 | 4.3 | 59.5 |

| | C18:1 | C18:2 | | C20:1 | C20:2 | | | % of |
|---|---|---|---|---|---|---|---|---|
| | Δ11 | Δ9, 12 | C20:0 | Δ11 | Δ11, 14 | C22:0 | C24:0 | dew |
| Polar lipid | | | | | | | | |
| 20 h | 0.9 | 14.6 | 0.0 | 0.2 | 0.0 | 0.0 | 0.3 | 0.4 |
| 1 d | 1.1 | 15.1 | 0.0 | 0.2 | 0.0 | 0.0 | 0.2 | 1.8 |
| 2 d | 2.0 | 13.2 | 0.0 | 0.1 | 0.0 | 0.0 | 0.2 | 3.3 |
| 3 d | 3.2 | 12.8 | 0.0 | 0.1 | 0.0 | 0.0 | 0.2 | 2.0 |
| 4 d | 3.7 | 12.4 | 0.0 | 0.2 | 0.0 | 0.0 | 0.3 | 1.8 |
| 5 d | 3.7 | 11.3 | 0.0 | 0.4 | 0.1 | 0.0 | 0.4 | 1.6 |
| 6 d | 3.0 | 15.7 | 0.0 | 0.9 | 0.2 | 0.0 | 0.5 | 1.9 |
| 7 d | 2.7 | 20.2 | 0.0 | 0.8 | 0.2 | 0.0 | 0.2 | 1.2 |
| Triacylglycerols | | | | | | | | |
| 20 h | 0.8 | 13.6 | 0.2 | 0.0 | 0.0 | 0.4 | 3.5 | 0.1 |
| 1 d | 0.9 | 8.8 | 0.9 | 0.0 | 0.0 | 1.0 | 8.2 | 0.2 |
| 2 d | 1.1 | 7.9 | 0.5 | 0.1 | 0.0 | 0.7 | 7.3 | 0.7 |
| 3 d | 1.4 | 7.1 | 0.6 | 0.0 | 0.0 | 0.8 | 8.6 | 0.6 |
| 4 d | 1.7 | 7.2 | 0.5 | 0.2 | 0.0 | 0.6 | 8.1 | 0.8 |
| 5 d | 1.9 | 6.8 | 0.4 | 0.4 | 0.0 | 0.4 | 5.4 | 1.1 |
| 6 d | 2.7 | 7.9 | 0.3 | 0.6 | 0.1 | 0.4 | 4.2 | 1.1 |
| 7 d | 2.8 | 7.7 | 0.2 | 0.4 | 0.1 | 0.2 | 1.7 | 0.9 |

TABLE 8
Fatty acid composition of polar lipid and TAG fractions from S. cerevisiae
strains INVSc1 and D5A during culturing in YPD medium for up to 7 days.

| | | | | | | | | | |
|---|---|---|---|---|---|---|---|---|---|
| | | | | C16:1 | | | C18:1 | C18:2 | % of |
| C12:0 | C14:0 | C15:0 | C16:0 | Δ9 | C18:0 | C18:1Δ9 | Δ11 | Δ9, 12 | dew |

Polar lipid - strain INVSc1

| | | | | | | | | | | |
|---|---|---|---|---|---|---|---|---|---|---|
| 20 h | 1.0 | 1.4 | 0.5 | 17.6 | 54.8 | 2.2 | 21.2 | 1.3 | 0.0 | 1.0 |
| 1 d | 0.7 | 0.8 | 0.6 | 13.2 | 54.8 | 3.0 | 25.1 | 1.9 | 0.0 | 0.8 |
| 2 d | 0.3 | 0.5 | 0.6 | 11.1 | 52.9 | 4.3 | 28.2 | 2.2 | 0.0 | 1.0 |
| 3 d | 0.2 | 0.4 | 0.4 | 11.7 | 51.0 | 4.8 | 29.9 | 1.6 | 0.0 | 0.8 |
| 4 d | 0.1 | 0.4 | 0.4 | 11.5 | 50.4 | 5.1 | 30.4 | 1.6 | 0.0 | 0.9 |
| 5 d | 0.1 | 0.4 | 0.5 | 12.0 | 49.7 | 5.4 | 30.3 | 1.6 | 0.0 | 0.7 |
| 6 d | 0.1 | 0.3 | 0.5 | 11.1 | 49.6 | 5.5 | 31.3 | 1.7 | 0.0 | 0.8 |
| 7 d | 0.1 | 0.3 | 0.5 | 10.9 | 49.0 | 5.8 | 31.8 | 1.6 | 0.0 | 0.8 |

Triacylglycerols - strain INVSc1

| | | | | | | | | | | |
|---|---|---|---|---|---|---|---|---|---|---|
| 20 h | 1.7 | 2.7 | 0.6 | 14.0 | 58.2 | 3.7 | 17.7 | 1.4 | 0.0 | 0.3 |
| 1 d | 0.9 | 1.3 | 0.6 | 9.5 | 56.2 | 5.5 | 23.7 | 2.4 | 0.0 | 0.3 |
| 2 d | 0.3 | 0.9 | 0.6 | 10.8 | 49.2 | 5.9 | 30.4 | 1.9 | 0.0 | 0.5 |
| 3 d | 0.2 | 1.2 | 0.5 | 13.9 | 45.2 | 6.1 | 31.5 | 1.4 | 0.0 | 1.2 |
| 4 d | 0.2 | 1.0 | 0.5 | 14.1 | 45.3 | 6.5 | 30.9 | 1.4 | 0.0 | 1.5 |
| 5 d | 0.4 | 2.0 | 1.1 | 16.2 | 48.8 | 5.5 | 24.5 | 1.4 | 0.0 | 1.5 |
| 6 d | 0.1 | 0.7 | 0.5 | 14.0 | 45.3 | 7.2 | 30.6 | 1.5 | 0.0 | 1.6 |
| 7 d | 0.1 | 0.7 | 0.5 | 13.5 | 45.3 | 7.5 | 30.8 | 1.5 | 0.0 | 3.0 |

Polar lipid - strain D5A

| | | | | | | | | | | |
|---|---|---|---|---|---|---|---|---|---|---|
| 20 h | 0.4 | 1.9 | 0.2 | 19.5 | 49.3 | 2.1 | 25.6 | 0.8 | 0.0 | 0.8 |
| 1 d | 0.5 | 1.8 | 0.2 | 19.1 | 48.2 | 2.4 | 26.5 | 1.1 | 0.0 | 0.9 |
| 2 d | 0.3 | 1.2 | 0.3 | 15.6 | 46.5 | 3.6 | 31.0 | 1.5 | 0.0 | 1.2 |
| 3 d | 0.2 | 0.7 | 0.2 | 11.8 | 44.4 | 4.9 | 36.2 | 1.6 | 0.0 | 1.2 |
| 4 d | 0.1 | 0.5 | 0.2 | 10.5 | 41.6 | 5.8 | 39.6 | 1.7 | 0.0 | 1.5 |
| 5 d | 0.1 | 0.4 | 0.1 | 10.7 | 38.4 | 6.6 | 42.0 | 1.6 | 0.0 | 1.4 |
| 6 d | 0.1 | 0.4 | 0.2 | 9.8 | 37.8 | 7.1 | 43.2 | 1.5 | 0.0 | 1.3 |
| 7 d | 0.1 | 0.4 | 0.2 | 9.4 | 37.4 | 7.3 | 43.8 | 1.5 | 0.0 | 1.3 |

Triacylglycerols - strain D5A

| | | | | | | | | | | |
|---|---|---|---|---|---|---|---|---|---|---|
| 20 h | 1.4 | 3.6 | 0.2 | 11.4 | 50.7 | 2.2 | 29.7 | 0.8 | 0.0 | 2.5 |
| 1 d | 1.4 | 3.3 | 0.2 | 10.1 | 52.5 | 2.3 | 29.4 | 0.9 | 0.0 | 2.3 |
| 2 d | 0.7 | 2.0 | 0.2 | 10.7 | 50.4 | 4.0 | 30.5 | 1.4 | 0.0 | 2.6 |
| 3 d | 0.3 | 1.0 | 0.2 | 10.0 | 48.9 | 4.7 | 33.2 | 1.7 | 0.0 | 2.2 |
| 4 d | 0.2 | 0.7 | 0.2 | 8.4 | 48.5 | 4.7 | 35.7 | 1.6 | 0.0 | 2.3 |
| 5 d | 0.1 | 0.6 | 0.1 | 8.6 | 43.8 | 5.8 | 39.3 | 1.6 | 0.0 | 3.5 |
| 6 d | 0.1 | 0.5 | 0.1 | 7.9 | 44.1 | 5.5 | 40.2 | 1.5 | 0.0 | 3.2 |
| 7 d | 0.1 | 0.5 | 0.1 | 7.6 | 43.8 | 5.5 | 40.8 | 1.5 | 0.0 | 2.8 |

TABLE 9
Fatty acid composition of TAG fraction from Y. lipolytica strains cultured in DM-Gluc
medium for 48 h. Triplicate cultures were analysed, except for strain CS5411.

| | | | | | | C16:1 | C16:1 | | |
|---|---|---|---|---|---|---|---|---|---|
| Strain | C12:0 | C12:1 | C14:0 | C15:0 | C16:0 | Δ9 | Δ7 | C17:1 | C18:0 |
| CS4813 | 0.1 | 0.0 | 0.7 | 0.4 | 17.0 | 4.4 | 0.4 | 1.5 | 24.0 |
| | 0.0 | 0.0 | 0.7 | 0.4 | 16.1 | 4.4 | 0.3 | 1.5 | 22.4 |
| | 0.0 | 0.0 | 0.6 | 0.4 | 15.2 | 4.5 | 0.3 | 1.6 | 21.1 |
| CS4814 | 0.0 | 2.7 | 0.6 | 0.3 | 14.6 | 5.9 | 1.6 | 1.8 | 14.2 |
| | 0.0 | 1.3 | 0.4 | 0.3 | 16.0 | 5.3 | 1.3 | 1.6 | 18.7 |
| | 0.0 | 0.2 | 0.5 | 0.3 | 16.7 | 6.2 | 1.3 | 1.6 | 18.9 |
| CS5933 | 0.3 | 0.0 | 0.5 | 0.3 | 12.2 | 6.0 | 0.6 | 1.6 | 16.8 |
| | 0.1 | 0.7 | 0.3 | 0.2 | 11.6 | 5.9 | 0.6 | 1.6 | 15.6 |
| | 0.0 | 3.5 | 0.5 | 0.2 | 10.2 | 6.4 | 0.7 | 1.8 | 11.9 |
| CS5411 | 0.0 | 0.6 | 0.4 | 0.2 | 14.2 | 3.5 | 0.8 | 2.1 | 20.4 |
| W29 | 0.0 | 0.8 | 0.2 | 0.2 | 15.1 | 3.7 | 2.7 | 0.8 | 16.8 |
| | 0.4 | 0.0 | 0.5 | 0.2 | 16.7 | 3.8 | 1.4 | 0.9 | 23.0 |
| | 0.0 | 0.7 | 0.2 | 0.3 | 15.0 | 4.0 | 1.9 | 0.9 | 18.5 |
| | | | | | | | | | TAG |
| | C18:1 | C18:1 | C18:2 | | C20:1 | C20:1 | | | content |
| | Δ9 | Δ11 | Δ9, 12 | C20:0 | Δ11 | Δ8 | C22:0 | C24:0 | (%) |
| CS4813 | 30.6 | 0.0 | 4.9 | 1.6 | 0.1 | 7.0 | 1.4 | 5.5 | 1.2 |
| | 33.5 | 0.5 | 4.2 | 1.5 | 0.1 | 7.7 | 1.3 | 5.0 | 1.6 |
| | 35.2 | 0.6 | 4.0 | 1.5 | 0.1 | 8.4 | 1.3 | 4.9 | 1.0 |
| CS4814 | 39.0 | 0.6 | 5.3 | 0.7 | 0.2 | 7.5 | 0.9 | 4.0 | 1.8 |
| | 35.6 | 0.4 | 5.6 | 0.9 | 0.1 | 7.1 | 1.1 | 4.2 | 1.7 |
| | 35.6 | 0.5 | 5.9 | 0.9 | 0.1 | 6.1 | 1.1 | 4.1 | 1.9 |
| CS5933 | 41.5 | 1.1 | 5.1 | 1.4 | 0.2 | 7.3 | 1.1 | 3.6 | 1.9 |
| | 42.1 | 1.2 | 5.4 | 1.4 | 0.3 | 8.0 | 1.1 | 3.6 | 2.0 |
| | 44.8 | 1.7 | 4.9 | 1.1 | 0.3 | 8.0 | 0.9 | 2.6 | 1.9 |
| CS5411 | 38.2 | 0.4 | 5.6 | 1.3 | 0.1 | 6.9 | 1.3 | 3.5 | 2.4 |
| W29 | 36.0 | 0.0 | 6.9 | 0.8 | 0.1 | 9.7 | 1.0 | 4.9 | 1.6 |
| | 33.0 | 0.0 | 6.2 | 1.0 | 0.1 | 6.8 | 1.1 | 4.8 | 1.7 |
| | 37.4 | 0.0 | 5.7 | 0.9 | 0.1 | 8.0 | 1.0 | 5.4 | 1.9 |

The TAG samples also contained 0.0%, 0.1% or 0.2% of C20:2ω6 and C22:2ω6

### Example 4. Larger Scale Production of Lipid in Microbes

A series of experiments was undertaken to investigate culturing of Y. lipolytica at larger scale in biofermenters, specifically at volumes of at least 25 L. Initial experiments used the wild-type Y. lipolytica strain W29, with extraction of lipid from the harvested cell mass using organic solvents.

### Experiment 1 (B001)

In a first experiment at a 25 L scale, wild-type Y. lipolytica strain W29 was grown in a 25 L fermenter to test biomass production, recovery and drying, and lipid extraction processes from a batch culture at the larger scale. Lipid accumulation was also monitored at different time points in this culture. The growth medium was as described in Table 1 of the review by Hahn-Hagerdal et al. (2005), DM column, page 5 with the following adjustments: all vitamins were omitted except thiamine. Potassium dihydrogen phosphate was added at 10 g/L, ammonium sulphate was replaced with diammonium phosphate at 10 g/L, citric acid was added at 2 g/L. The trace elements CuSO₄, NaMoO₄, MnCl₂, CoCl₂, H₃BO₃ and ZnSO₄ were present in reduced concentrations to the published recipe by varying amounts of between 3 and 30-fold and CaCl₂) was increased by a factor of 8. Additional S, N and P were supplied as inorganic acids. The culture medium was sterilised in the fermenter by autoclaving and thiamine added aseptically after the heat treatment to a final concentration of 0.15 g/L using a 200 g/L sterile filtered thiamine stock solution. The growth medium had 40 g/kg glycerol as the carbon source, pH 6.0 at the beginning of culture. One advantage of this medium was that it could be sterilised by autoclave as a whole, with addition after autoclaving of only the thiamine.

The strain W29 inoculum for the fermenter culture was grown as a 400 ml culture in YPD medium, in flasks at 29° C. with shaking at 180 rpm for 24 h. The inoculum was added to the fermenter and the mixture sampled to provide a time zero sample. After inoculation, the OD600 of the culture was 0.132. The culture conditions were: temperature at 29° C., the pH set point was 6.0, the airflow was approximately 33 L/min and the stirrer approximately 200 RPM. The following parameters were monitored—dissolved oxygen (DO) concentration and pH. The temperature and pH values were controlled to the respective set-points. The culture pH value was changed from 6.0 to 8.0 at the 47 h time-point (post inoculation) to stimulate the accumulation of lipid. Growth of the yeast was monitored by measuring OD at 600 nm (OD600). The level of citric acid in the medium was also monitored since wild-type Y. lipolytica secretes citrate during growth on glycerol as carbon source. The DO decreased during culture from 10 ppm to about 2 ppm at 48 h. A metabolite with the HPLC retention time consistent with citric acid was produced in the culture and accumulated gradually, reaching about 40 g/L at 60 h but then declining to about 33 g/L at 90 h. The concentration of glycerol decreased gradually to about zero at 40 h, at which time a glycerol feed was supplied to the culture using 4.5 L of 400 g/kg glycerol over the next 8 h. This increased the glycerol concentration in the medium to about 20 g/kg, after which the glycerol concentration decreased to zero at 60 h timepoint. At 90 h timepoint, the cell density had reached about 30 g/kg (DCW), at which time the culturing was ceased and the cells harvested by centrifugation. The biomass was washed with 2 volumes of cold water, providing a yeast cream of 3.2 kg having 17% solids. Half of the biomass was spray dried with an inlet temperature of 160° C., outlet temperature of 78° C., yielding 156 g of dried powder. The remaining 1.7 kg having 20% solids was frozen. A small portion of this material was freeze dried, recovering 22 g of dried cells.

Samples were removed at 42, 62 and 68 h during the culturing and at 90 h at harvest of the cells. The samples were either spray dried or freeze dried and analysed for lipid content by extraction of lipid using ethanol/hexane (60/40; v/v) as solvent for 20 h. The solvent of the extracted lipid was evaporated under vacuum at 50° C., and the lipid dried under a stream of CO₂. The dried lipid was weighed. At each of the timepoints from 40 h to 90 h, the amount of recovered lipid was between 17-25% on a dry cell weight basis. To analyse the composition, samples of the extracted lipid were dissolved in 2 ml of ethanol/hexane (6/4 v/v) or 1 ml chloroform and 2×5 μl aliquots chromatographed on a TLC plate (Silica gel 60 F254, 25 cm×25 cm) using hexane/diethylether/acetic acid (70/30/1; v/v/v). The plates were stained with iodine vapor for 30 min to observe the lipid types. Bands were observed for polar lipids at the origin of the TLC plate, and, with increasing mobility, for DAG, free fatty acids (FFA) and TAG, with the TAG bands by far the most intense.

In this experiment, 2.8 kg of glycerol was fed to the culture after the initial batch phase had ended as determined by the exhaustion of glycerol at around 40 hours post inoculation. Almost equal quantities of citric acid and biomass were produced by the end of the experiment at 90 h post inoculation. Interestingly, the highest level of extracted lipids per gram of cell dry matter occurred at the point when glycerol from the nutrient feed was exhausted at 61.5 h post inoculation. Further lipid generation was not observed between this point and when the experiment ended at 90 h. This fermentation produced a lower cell density than experienced at a 2 L scale but significantly higher TAG accumulation at about 30% vs 5% on a dry cell weight basis. This experiment out to 90 h was designed for large amounts of TAG production after nitrogen limitation was achieved. Improvements were considered where cell density was increased whilst maintaining good TAG production, most likely through balancing the nitrogen and glycerol concentrations. The concentration of citric acid declined late in the fermentation suggesting this can be used as a carbon source when glycerol is exhausted.

It was demonstrated that the yeast cells could be successfully harvested by centrifugation and that culture broth could be removed from the cells by resuspending the cell pellet in cold water and then re-centrifuging the cells to a paste. It was also demonstrated that the biomass could be freeze dried from frozen paste or spray dried from a yeast cream of 20% solids. It was concluded from the TLC analysis that at least 30% of the solvent extract from the cells was TAG, the remaining lipid mass made up by DAG>FFA and polar lipid, in that order of abundance.

### Experiments 2 and 3 (B003, B004)

In further experiments at the 25 L scale, wild-type Y. lipolytica strain W29 was grown in a medium using 40 g/L or 70 g/L glycerol as carbon source in order to assess biomass production and lipid production at various C/N ratios. In experiment B003, the initial medium had 40 g/L glycerol, thiamine at 0.15 g/L, pH 6.0 and no added citric acid, relative to experiment B001. As nitrogen source, di-ammonium phosphate (DAP) was present at 10 g/L to encourage biomass growth during the batch stage. The initial C:N ratio was therefore initially 6:1. The initial cell density (OD600) after addition of the 1 L inoculum of W29 cells was 0.22 and the pH 6.22. The biomass reached a dew of 15.7 g/L at 17 h. At 20-hours post inoculation, the feed (5 L) including glycerol and DAP was started at 0.5 L per hour, providing a C:N ratio of 20:1. The biomass continued to grow in exponential phase to 30.51 g/L at 24 h, equal to half of the feed, indicating there was remaining nitrogen in the batch medium at 20 h. After that the biomass reached stationary phase suggesting that a critical nutrient was limited. As there was excess glycerol and citric acid in the 24 h culture sample, the limiting nutrient was likely to be nitrogen. From the 24 h time-point, the citric acid concentration increased to a maximum of 14 g/L while glycerol was steadily consumed to a minimum of 0.46 g/L by the 30 h time-point when the feed medium was consumed. At the end of the fermentation experiment at 45 h, the DCW was 34.3 g/L, and the citric acid concentration was 7.69 g/L. In contrast to experiment B001, the harvest biomass DCW was increased from 29.6 to 34.3 g/L and the citric acid concentration in the harvest supernatant reduced from 33.45 to 7.69 g/L. However, the ethanol/hexane extractable lipid content was reduced from 28% in B001 to 8% in B003. The recovered cells were washed with 2 volumes of cold 1% NaCl (w/v) and spray dried. In conclusion, 671 g of washed yeast powder was produced from 30 L of fermenter broth. Nutrients were more efficiently converted to biomass than in experiment B001 but less lipid accumulated. Prolonged fermentation was expected to increase the lipid yield.

Experiment B004 modified the initial medium to 70 g/L glycerol, to provide for earlier nitrogen limitation between 24-30 h before the glycerol feed began. The feed included a mineral salt mixture with some nitrogen, as the sole nitrogen source. The DAP was omitted from the feed in order to increase the C:N ratio from 20:1 to approximately 100:1. A change in oxygen consumption or glycerol depletion was monitored to signal the start of the feed. A biomass of 50.1 g/L was achieved at the 32 h timepoint. At the end of Feed 1, the residual glycerol and citric acid were 18 g/L and 15 g/L, respectively. Batch fermentation after Feed 1 continued to 47 h. 7 L broth was harvested, concentrated, washed and frozen. It was noted that 50% of the cells stained with methylene blue after the extended cultivation at pH 6 after Feed 1. The dissolved oxygen level rose dramatically indicating that the culture was metabolically inactive, most likely due to nitrogen exhaustion.

As there was a significant population of live cells, a second feed started with the same composition as Feed 1, but the pH was adjusted to 8 to investigate if this might result in lipid storage in the remaining viable cells. After the second feed, the biomass increased slightly from 43 to 47 g/L. The residual glycerol and citric acid continually increased to 23 g/L and 25 g/L, respectively, during the second feed. The batch fermentation continued for another 16 h after the end of Feed 2. By the end of the experiment, citric acid had increased to 40 g/L and the residual glycerol had reduced to 9 g/L. The fermentation was stopped at 77 h. 7 L of the culture was harvested before heat treatment. The biomass had a dry weight of 41 g/L with a neutral lipid content of 18% w/w, and the glycerol and citric acid were 9 g/L and 40 g/L respectively. The remaining culture was treated by heating to 105° C. for 5 min to kill the cells.

It was concluded from these experiments that large scale fermentation could produce yeast cells with suitable biomass and lipid production.

### Example 5. Extraction and Fractionation of Lipid—Larger Scale

The following experiments were performed to test extraction of lipid and purification/fractionation of lipid, including TAG and phospholipid, at a larger scale from Y. lipolytica cells grown at volumes of at least 2 L or 8 L in a fermenter.

### Experiment 1

The inventors considered that lipid could be extracted from microbial cells such as Y. lipolytica and simultaneously separate TAG and polar lipid by using a solvent mixture of ethanol:hexane. This method was based on Sun et al. (2019). About 100 g wet weight of Y. lipolytica cells were used, testing whether the TAG and polar lipids could be effectively extracted from the cells and partitioned between a hexane phase and an ethanol-water phase, respectively. In this experiment, 102.12 g wet weight of cells, corresponding to 20.42 g dry weight of cells i.e. about 80% moisture and 20% solids, was mixed overnight with 500 ml of ethanol/hexane (4/6, v/v) by stirring, in a 1 L beaker. This provided a sample/solvent ratio of 1/5 (w/v) based on wet weight, or 1/25 (w/v) based on the dry weight of cells. After the overnight mixing, the mixture had separated into two phases with flocculated cellular material at the interface. The upper, hexane phase was pale yellow in colour, while the lower, ethanol phase was pale green. The mixture was decanted and filtered through a glass fibre filter (1.2 μm, MicroAnalytix Pty Ltd, Catalog No. WH1822-090) using a glass vacuum filtration apparatus to remove the flocculated cellular material, and the residue rinsed with 100 ml of ethanol/hexane (4/6 v/v) solvent, combining the filtrates. The filtrate was separated into two phases in a separatory funnel: an upper, hexane phase containing what was hoped to have most of the TAG and a lower ethanol phase containing what was hoped to have most of the polar lipid including PL. The two phases were collected separately in 1 L round bottom flasks and dried using a rotary evaporator. The dried extracts were weighed, yielding 0.513 g of extracted lipid from the hexane phase (2.510% of the 20.42 g dry cell weight) and 3.69 g of extracted lipid from the ethanol phase (18.06% w/dcw). Both fractions were washed with chloroform to remove any traces of water, adding the chloroform followed by rotary evaporation, repeating this step until addition of chloroform resulted in a clear water-free extract. This resulted in recovery of 0.51 g of lipid from the upper phase and 3.43 g lipid from the ethanol phase.

To transfer the extracted lipid to a tube suitable for transport, the polar lipid extract was dissolved in 26 ml chloroform, transferred to a 50 ml plastic centrifugation tube and the solvent evaporated using a Savant SC250EXP SpeedVac concentrator at 45° C. overnight.

Samples of the extracted lipid fractions were applied to a TLC plate and chromatographed to separate the different lipid classes. As determined by quantitation of FAME, the extract from the upper, hexane phase contained 24.9% lipid, 60% of which was TAG but also containing substantial polar lipid, at 40% of the lipid. The extract from the lower, ethanol phase had 9.5% lipid, of which 95% was polar lipid, and no TAG was detected in that fraction.

To determine the amount of total extractable lipid from Y. lipolytica cells grown under the same conditions, using an established method, and to test whether the ethanol/hexane extraction was efficient enough, lipid was extracted from a similar quantity of cells using a standard procedure based on Bligh and Dyer (1959). Briefly, a frozen, wet cell pellet of 103.08 g having about 20% total solids was placed in a 1 L glass beaker. Chloroform/methanol/water solvent was added comprising 166.7 ml chloroform, 266.3 ml methanol and 53.4 ml water. The frozen pellet was broken into small pieces in the solvent using a spoon. The beaker was covered with aluminium foil and the mixture stirred overnight at room temperature with a magnetic stirrer. The mixture was then vacuum filtered through a glass fibre filter, and the residue of cellular material rinsed with 131 ml chloroform and 103 ml water and filtered again. The total filtrate was transferred to a 1 L separation funnel, shaken gently and allowed to stand for several hours for phase separation to occur. The bottom chloroform layer containing extracted lipid was drained into a 250 ml round bottom flask and the solvent removed by rotary vacuum evaporation. The total lipid extract in the flask was weighed for gravimetric yield determination: the lipid yield of 2.06 g was 9.97% on a dry cell weight basis assuming an 80% moisture content of the wet yeast cell pellet. The total lipid extract was dissolved in 26 ml chloroform and samples converted to FAME and quantitated by GC as described in Example 1.

Samples of the extracted lipid from the Bligh-Dyer method were also applied to a TLC plate and chromatographed to separate the different lipid classes. Standards of known lipid classes were applied to adjacent lanes to identify the lipid spots. This identified the polar lipid and TAG fractions in the Bligh-Dyer extract and allowed their quantification. The Bligh Dyer extract contained 28.3% total lipid, 7% of which was TAG and 93% of which was polar lipid, The extraction using the ethanol/hexane method was nearly as efficient as the Bligh-Dyer method, demonstrating that the ethanol/hexane solvent system was useful.

Samples of each of the fractions were analysed for fatty acid composition by conversion to FAME and GC analysis as described in Example 1, for the extracts from the upper (hexane) and lower (ethanol) phases. The fatty acid composition was also separately determined for the polar lipid and TAG fractions isolated on TLC plates. The data are provided in Table 10. In Experiment 1, the lipid extracted in the lower (ethanol) phase contained predominantly (95%) polar lipid, having a fatty acid composition much lower in the saturated fatty acid C18:0, C22:0 and C24:0 than the TAG extracted in the upper (hexane) phase. In contrast, the level of C16:0 was similar in both the polar lipid and TAG fractions from the same upper phase at between 16-18%.

One conclusion from this experiment was that the lipid extracted from the lower, ethanol phase was almost entirely polar lipid, essentially lacking TAG. However, this method did not extract all of the polar lipid available, leaving considerably polar lipid in the upper hexane phase. The remaining polar lipid can be recovered by degumming procedures. Although the extracted material from the ethanol phase was only 9.5% lipid by weight, the other 90% of the material, presumably ethanol soluble substances such as some proteins and carbohydrates, can be removed to some extent by extraction of the lipids into chloroform, since proteins and carbohydrates are not soluble in chloroform.

TABLE 10
Fatty acid composition of the extracted lipid in fractions from Y. lipolytica (W29)
in Experiments 1 and 2, compared to Bligh Dyer extraction. The fatty acid composition
is shown for the total lipid (Total) and the polar lipid (Polar) and TAG fractions
in each extract, from the top phase (hexane) or bottom phase (ethanol).

| Fraction/ | | | | | C16:1 | C16:1 | | | C18:1 |
|---|---|---|---|---|---|---|---|---|---|
| experiment | | C14:0 | C15:0 | C16:0 | Δ7 | Δ9 | C17:1 | C18:0 | Δ9 |
| Upper phase - | Total | 0.2 | 0.3 | 17.1 | 0.5 | 7.5 | 0.8 | 8.4 | 33.0 |
| Experiment 1 | Polar | 0.4 | 0.6 | 16.6 | 0.5 | 8.7 | 1.2 | 2.0 | 35.7 |
| | TAG | 0.3 | 0.4 | 17.9 | 0.5 | 6.9 | 0.9 | 13.5 | 27.9 |
| Lower phase - | Total | 0.2 | 0.4 | 16.6 | 0.5 | 9.1 | 1.4 | 1.6 | 36.3 |
| Experiment 1 | Polar | 0.4 | 0.7 | 17.4 | 0.6 | 9.2 | 1.2 | 1.9 | 34.8 |
| Upper phase - | Total | 0.2 | 0.3 | 18.0 | 0.5 | 6.4 | 0.8 | 11.6 | 29.8 |
| Experiment 2 | Polar | 0.3 | 0.6 | 18.6 | 0.4 | 5.7 | 0.9 | 2.1 | 35.2 |
| | TAG | 0.3 | 0.4 | 18.0 | 0.5 | 6.7 | 0.9 | 14.2 | 28.4 |
| Lower phase - | Total | 0.3 | 0.5 | 16.5 | 0.4 | 8.8 | 1.1 | 2.0 | 35.4 |
| Experiment 2 | Polar | 0.4 | 0.7 | 16.8 | 0.6 | 9.3 | 1.3 | 1.8 | 35.2 |
| | TAG | 1.0 | 2.6 | 21.7 | 0.4 | 5.4 | 0.6 | 18.0 | 24.7 |
| Bligh-Dyer- | Total | 0.3 | 0.5 | 17.0 | 0.5 | 8.4 | 1.1 | 2.7 | 35.1 |
| Experiment 1 | Polar | 0.4 | 0.6 | 16.9 | 0.6 | 8.7 | 1.2 | 1.9 | 35.6 |
| | TAG | 0.4 | 0.6 | 18.8 | 0.5 | 7.1 | 0.9 | 13.0 | 28.0 |

| Fraction/ | | C18:1 | | | C18:3 | | | % | |
|---|---|---|---|---|---|---|---|---|---|
| experiment | | Δ11 | C19:0 | C18:2 | ω3 | C22:0 | C24:0 | lipid | g total |
| Upper phase - | Total | 0.5 | 0.1 | 30.3 | 0.0 | 0.2 | 1.6 | 24.9 | |
| Experiment 1 | Polar | 0.5 | 0.1 | 33.3 | 0.1 | 0.0 | 0.3 | 9.0 | 0.062 |
| | TAG | 0.5 | 0.1 | 26.7 | 0.1 | 0.6 | 3.8 | 13.1 | 0.091 |
| Lower phase - | Total | 0.5 | 0.1 | 33.8 | 0.0 | 0.0 | 0.1 | 9.5 | |
| Experiment 1 | Polar | 0.5 | 0.1 | 33.0 | 0.1 | 0.0 | 0.0 | 9.0 | 0.616 |
| Upper phase - | Total | 1.6 | 0.1 | 28.1 | 0.0 | 0.4 | 2.3 | 37.4 | |
| Experiment 2 | Polar | 0.4 | 0.1 | 35.1 | 0.0 | 0.0 | 0.4 | 6.6 | 0.068 |
| | TAG | 0.6 | 0.1 | 25.7 | 0.0 | 0.6 | 3.5 | 26.8 | 0.273 |
| Lower phase - | Total | 1.1 | 0.2 | 33.4 | 0.0 | 0.0 | 0.3 | 12.5 | |
| Experiment 2 | Polar | 0.5 | 0.1 | 33.3 | 0.1 | 0.0 | 0.0 | 11.4 | 0.260 |
| | TAG | 0.4 | 0.1 | 21.1 | 0.0 | 0.6 | 3.3 | 0.3 | 0.007 |
| Bligh-Dyer- | Total | 0.7 | 0.1 | 32.9 | 0.0 | 0.0 | 0.3 | 28.3 | |
| Experiment 1 | Polar | 0.5 | 0.1 | 33.3 | 0.0 | 0.0 | 0.2 | 25.1 | 0.681 |
| | TAG | 0.5 | 0.1 | 27.0 | 0.0 | 0.5 | 2.6 | 2.0 | 0.054 |

### Experiment 2

A second experiment was performed which was the same as Experiment 1 except with a modification to the solvent used. This time, extraction of 99.8 g of wet weight cells initially used 500 ml of ethanol:hexane (6/4 v/v) rather than (4/6 v/v), with subsequent adjustment to (4:6 v:v). This modification was made because, in Experiment 1, the phases tended to separate even at the start of the extraction, possibly due to the amount of moisture in the cell sample. In this second experiment, the cells were mixed with the ethanol/hexane (6/4 v/v) by stirring at room temperature overnight. After that, 250 ml of hexane was added so that the solvent mixture was now ethanol/hexane (4/6 v/v), with mixing for a further 5 min. The rest of the procedure was the same as for Experiment 1. Product was initially recovered from the upper, hexane phase at 1.23 g (6.16% w/dcw) and from the lower, ethanol phase at 4.05 g (20.29% w/dcw). After the chloroform wash and drying of the extracts, the recoveries were 6.16 g and 19.89 g, respectively.

It was concluded from these experiments and the following ones that this solvent extraction method worked quite well as long as there was adequate agitation of the mixture during the overnight extraction. Conditions are varied for optimisation, e.g. solvent volumes, ratios, extraction time and temperature, and starting with wet vs dry cells. It was expected that extraction from dry cells and at a higher temperature would provide a greater yield of total lipid, TAG and polar lipid fractions.

### Experiment 3

Since the batch extractions in Experiments 1 and 2 used large amounts of solvent for the amount of recovered lipid at about 500 ml per 100 g wet weight of cells, it was decided to test extraction using a Soxhlet apparatus (De Castro et al., 2010). This experiment used the same solvent composition as in Experiment 2, starting with ethanol/hexane (6/4, v/v) and then adding hexane to adjust the ratio to (4/6, v/v). A cell sample of 20 g (wet weight) having 4 g dry weight of cells was added to a Soxhlet cup. Extraction used 300 ml ethanol/hexane (6/4, v/v) in the flask, heating the solvent for 3 h using a heating mantle, and cooling of the condenser with running tap water. After the 3 h extraction, the flask was rinsed with 150 ml of hexane, thereby adjusting the solvent ratio to ethanol/hexane (4/6, v/v). The remainder of the procedure was the same as for Experiment 1, with recovery of the lipids from the upper, hexane phase and the lower, ethanol phase. The recovery of lipid from the upper phase was 5.4% (w/dcw) and from the lower phase 17% (w/dcw), so almost the same polar lipid yield was obtained compared to Experiments 1 and 2, and in a shorter time. It was considered that this method had potential for scaling up by using a larger 1 kg or 5 kg Soxhlet apparatus, or even larger pilot scale extractions.

### Experiment 4

A larger batch extraction was performed on 900 g (wet weight) of cells using the solvent system as in Experiment 2, with an initial ratio of ethanol/hexane at 6/4 (v/v), then adjusted to ethanol/hexane at 4/6 (v/v). The extraction was carried out in three 2 L flasks. The recoveries of the lipid from the upper phase was 10.89 g, mostly TAG, and from the lower phase 32.45 g, mostly polar lipid. This represented a 18% yield (w/w on a dew basis) of the extracted polar lipid. The polar lipid fraction was quite viscous but was able to be transferred from the evaporator flask by warming it to 50° C.

### Experiment 5

An experiment was carried out to compare the efficiency of lipid extraction from wet Y. lipolytica cells relative to freeze-dried cells in a powder form, using ethanol/hexane at 6/4 (v/v) at a warmer temperature of 50° C. compared to previous experiments carried out at room temperature. The extractions were done for 3 h or with a Soxhlet apparatus using the same solvent for 3 h. After the extraction was completed, the ratio of ethanol/hexane was adjusted to 4/6 (v/v) by addition of more hexane. The Y. lipolytica strain W29 cells had been grown in the presence of ARA to incorporate the ω6 fatty acid into polar lipids. The mixture of the solvent with the dried cells resulted in a single phase, whereas the corresponding mixture from the wet cells resulted in two phases due to the water content. Some water was therefore added to half of the former mixture which, after mixing, resulted in the separation of two phases. The mixtures were filtered to remove cell debris and lipid was recovered from each of the phases. The results are presented in Table 11. For the dry cells, lipid was recovered from the single-phase extract and separately from the two phases after the addition of some water.

### Experiment 6. Extraction of Lipids with Organic Solvents at Large Scale

A Y. lipolytica strain having fad2 and ura3 mutations (Example 7 below) and containing a genetic construct with three genes including a Ura3 gene was grown in a 10 L fermenter in 8 L of culture volume containing 8% glycerol (w/v) as the carbon source and having a lower nitrogen content (DM-Glyc-LowN medium, Example 1) to induce TAG production. The OD600 of the culture after 48 h in that medium was 139 and the dry cell yield was about 52 g/L. After the 48 h culturing, the entire culture was heat treated at 105° C. for 5 min to kill the cells. The cells were harvested by centrifugation, providing about 1.6 kg of cell paste (wet weight) having a dry cell weight content of about 26%. The harvested cells were split into samples to compare different extraction methods as follows.

TABLE 11
Yield of recovered lipid extracts from Y. lipolytica cells.

| | | | | | Total |
|---|---|---|---|---|---|
| | | Solvent | | Recovered | lipid |
| | Sample | mixture | Upper or | lipid | recovered |
| Extraction | mass | volume | lower | (% of | (% of |
| method | (g) | (ml) | phase | dcw) | dcw) |
| Wet cells/50° C. | 250.1 | 1250 | Upper | 3.6 | 14.4 |
| Batch extraction | | | Lower | 10.8 | |
| Wet cells/Soxhlet | 27.2 | 300 in | Upper | 3.0 | 12.7 |
| extraction | | reflux | Lower | 9.7 | |
| | | flask | | | |
| Dried cells/50° C. | 50.1 | 1250 | Single | 9.9 | 9.2 |
| Batch extraction | | | phase* | | |
| | | | Upper | 4.4 | |
| | | | Lower | 4.1 | |
| Dried cells/ | 15.0 | 300 in | Single | 6.9 | 6.7 |
| Soxhlet | | reflux | phase* | | |
| extraction | | flask | Upper | 3.5 | |
| | | | Lower | 3.1 | |

*Single phase refers to half of the volume (800 ml) from the dried cell extractions since there was no phase separation prior to adding water.

In a first method using ethanol/hexane as solvent, 381 g of cell paste were extracted with ethanol/hexane (60/40 v/v) using a solvent: wet cell weight ratio of about 5:1 (v/w), equivalent to about 20:1 (v/w) on a dry weight basis. This used 1144 ml ethanol and 762 ml hexane. The extraction was carried out at room temperature overnight with continuous stirring, after which the mixture was filtered to remove cell debris. The ethanol/hexane ratio of the liquid was adjusted to 40/60 (v/v) by addition of 950 ml of hexane and rinsing the extracted pellet. After another filtration to remove residual solids, the liquid phases were pooled and mixed and the phases allowed to separate in a separatory funnel. The upper (hexane) layer was collected and rotary evaporated at 40° C. at 25 mbar for 30 min to remove the hexane, providing an extracted lipid sample of 2.15 g. This represented an extracted yield of 2.16% on a dry cell weight basis. An aliquot of the extracted material was analysed by TLC to separate the lipid classes and quantitated by GC of FAME, as described in Example 1. The fatty acid content of this material was 39.2% by weight, including 22.6% of TAG by weight and 10.8% polar lipid by weight. It was a solid at room temperature, so not considered an oil but rather an extracted fat.

In a second method using hexane extraction after de-watering of the wet cell paste, 381 g of the wet cell paste was resuspended in 760 ml of ethanol and the cells recovered by filtration. The cells were resuspended in 760 ml of ethanol a second time and again recovered by filtration. This treatment was aimed at removing almost all of the water from the cell paste. The cells were then extracted with hexane (1.9 L) using a hexane:wet cell ratio of 5:1 (v/w), about 25:1 on a dry cell weight basis, stirring the mixture overnight at room temperature. The upper layer, about 95% of the total volume, was collected and rotary evaporating, providing an extracted lipid sample of 7.60 g. This represented an extracted yield of 7.67% on a dry cell weight basis. An aliquot of the extracted material was analysed by TLC to separate the lipid classes and quantitated by GC of FAME, as described in Example 1. The total fatty acid content of this sample was 37.1% by weight, including 24% of TAG by weight and 11.5% of polar lipid by weight. It was a solid at room temperature, so not considered an oil but rather an extracted fat.

In a third method using a DMSO/hexane solvent rather than ethanol/hexane, similar to the first method but using DMSO/hexane instead of ethanol/hexane, 60.1 g of wet cell paste was mixed with 20 ml DMSO and 300 ml hexane and stirred overnight at room temperature. The following steps were essentially the same as in the first method. This method gave a relatively poor lipid yield of about 0.5% on a dry cell weight basis. An aliquot of the extracted material was analysed by TLC to separate the lipid types and quantitated by GC of FAME, as described in Example 1. The fatty acid content of this sample was 43.5% by weight, including 19.9% of TAG by weight and 17.1% polar lipid by weight. This method clearly extracted less lipid, in particular less TAG, than the other two methods and was not favoured.

It was concluded that the second method was the more efficient of the tested methods and that extraction with hexane was suitable for extractions of 1 kg or more of microbial cells (wet or dry weight) such as yeast cells, including Y. lipolytica, provided that the water content of the cell mass was low enough.

The extracted lipid samples from the first and second methods were transferred to 10 ml glass tubes by dissolving the lipids in hexane, transferring the mixtures to the tubes and evaporating the hexane under a nitrogen stream. The extracted lipids were stored at room temperature after being flushed with nitrogen and sealed to prevent oxidation. To test for the presence of moisture or remaining solvent, samples were freeze dried overnight. By weighing the samples before and after, it was determined that the first extract had 14.5% water or solvent, whereas the second had 23.7% water or solvent. Once these were removed, the fatty acid content of the first extract was 46% on a weight basis and of the second extract 49%.

To further purify the lipid in the first and second extracts by removing at least some non-lipid compounds, the extracted lipids were dissolved in a volume of chloroform, then half a volume of methanol was added followed by 0.8 volume of aqueous 0.1 M KCl. This followed the solvent mixture of Bligh and Dyer (1959). Each solution was mixed thoroughly and two phases allowed to separate. The lower (chloroform) phase was collected. Another volume of chloroform/methanol was added to the upper phase in a second extraction, and the chloroform phases combined. The solvent was evaporated from each chloroform solution. The resultant lipid products had a total fatty acid content of 78% by weight in the first sample and 70% in the second sample as determined by GC analysis of FAME. Since these products were soluble in the organic solvents chloroform and hexane, they were considered to be pure lipid. The material that was removed by the purification step was considered to be material that was more soluble in the methanol/water phase than in chloroform, so probably included some proteins. The purified, extracted lipids were considered to comprise lipids other than TAG and polar lipid, such as sterols, sterol esters and some pigments. TAG was also purified from these samples by fractionation by TLC.

Fractionation of Polar and Non-Polar Lipids by Precipitation from Acetone.

An experiment was carried out to test whether lipid containing saturated and unsaturated fatty acids could be enriched for the saturated fatty acid content, using precipitation from an organic solvent at defined temperatures. To do this, 97 mg of cocoa butter (Societe Africaine De Cacao) and about 2 mg of polar lipid extracted from Y. lipolytica cells that had been cultured in the presence of ARA, having about 16.4% ARA in the total fatty acid content, were mixed in a 15 ml tube at 50° C. The lipid was dissolved in 5 ml of acetone by ultrasonication of the mixture in a water bath at 40° C. for 5 min and then mixing at 37° C. for 15 min. Cocoa butter was used because it is rich in saturated fatty acids. The lipid mixture in acetone was incubated at 20° C. with mixing for 24 h. No precipitate was clearly observed at this temperature. However, the mixture was centrifuged at 4,600 g for 15 min and the supernatant was transferred to a new tube, which was incubated at 15° C. for 24 h. The first tube was stored at −20° C. for lipid analysis of a small pellet that was observed. After centrifuging the mixture as before, the 15° C. supernatant was transferred to a new tube and incubated at 12.5° C. for 24 h, after which considerable precipitation was observed. The mixture was again centrifuged and the supernatant transferred to a new tube. The pellet was washed with 2 ml of cold (12.5° C.) acetone by gentle mixing and the supernatant was combined with the earlier supernatant, which was incubated at 10° C. for 3 days. After centrifugation, the supernatant was again transferred to a new tube, the pellet was washed with 2 ml cold acetone and the supernatant combined with the earlier supernatant, which was incubated at 4° C. for 24 h. After centrifugation, the supernatant was again collected in a new tube, the pellet was washed with 2 ml cold acetone and the supernatant was combined with the 4° C. supernatant. The acetone was evaporated from all of the pellets and supernatants under a flow of nitrogen at room temperature and the dried, recovered lipids were dissolved in chloroform. The TAG and polar lipids classes of the precipitates and supernatant fractions were separated by TLC chromatography using hexane/diethylether/acetic acid (70/30/1) and quantitated and analysed for fatty acid composition by GC of FAME.

The data are shown in Table 12. TAG and polar lipid were precipitated at all the temperatures tested, namely 20° C., 15° C., 12.5° C., 10° C. and 4° C. The greatest amount of TAG was precipitated at 12.5° C. (41%), followed by 21.9% at 4° C., while 30.7% of the polar lipid precipitated at 20° C. Most (60.9%) of the polar lipid remained in the supernatant at 4° C., with an enrichment of the level of ARA from 16.4% to 24.8% of the total fatty acid content of the polar lipid. The supernatant at 4° C. also contained 27.4% of the total TAG. Polar lipid precipitated at 20° C. and 15° C. were enriched in C18:1 and C18:2, while those from 12.5° C., 10° C. and 4° C. were enriched in C16:0 and C18:0 and contained lower proportions of ARA. On the other hand, the TAG that precipitated at the higher temperature contained higher levels of C18:0 whereas those from lower temperatures were richer in C16:0, C18:1 and C18:2. Precipitation from acetone or similar solvents can therefore be used to increase the SFA fatty acid content and to decrease the polar lipid/non-polar lipid (TAG) ratio in an extracted lipid. It can be used to increase the SFA content of extracted lipids, by removing PUFA and some MUFA and so enriching for SFA. Further optimization work can be done by seeding the mixture with TAG crystals to enhance TAG precipitation and application of low temperatures.

### Example 6. Modification of Microbes to Reduce Polyunsaturated Fatty Acids

Many yeasts produce polyunsaturated fatty acids (PUFA) including linoleic acid (LA, C18:2Δ9,12) and α-linolenic acid (ALA, C18:3Δ9,12,15) which are incorporated into their oil, including in TAG, and in their membrane lipids such as phospholipids. Production of LA and other PUFA derived from LA requires the activity of a Δ12 desaturase which is encoded by a FAD2 gene, whereas incorporation of the third double bond to produce ALA from LA additionally requires a Δ15 desaturase. When cultured in a rich medium lacking added fatty acids, the wild-type Y. lipolytica strain W29 produced the ω6 fatty acid LA (Example 3, Table 7). In some samples, strain W29 also produced trace amounts of the ω6 fatty acid C20:2Δ11,14 which was a two-carbon extension product of LA. Strain W29 appeared to lack a Δ15 desaturase since ALA was absent from the TAG and phospholipid. A FAD2 gene was cloned from Y. lipolytica by Yadav and Zhang (WO2004/104167) and Tezaki et al. (2017) and shown to encode the Δ12 desaturase. They also generated a deletion mutant (fad2) which did not produce LA. That mutant was compromised in its growth at 12° C. in the absence of added LA in the growth medium.

TABLE 12
Fatty acid composition of lipid precipitates (PPT) and supernatants (SUP) after acetone fractionation.

| | | | | | | | | | | C18:1 | C18:2 |
|---|---|---|---|---|---|---|---|---|---|---|---|
| | C12:0 | C12:1 | C14:0 | C15:0 | C16:0 | C16:1 | C17:1 | C18:0 | C18:1 | Δ11 | (LA) |
| Polar lipid | | | | | | | | | | | |
| fractions | | | | | | | | | | | |
| PPT 20° C. | 0.0 | 0.2 | 0.1 | 1.2 | 17.4 | 10.2 | 1.3 | 0.7 | 42.0 | 1.0 | 13.4 |
| PPT 15° C. | 0.4 | 4.9 | 0.4 | 1.2 | 18.3 | 8.6 | 1.0 | 7.2 | 33.6 | 0.0 | 9.8 |
| PPT 12.5° C. | 0.0 | 10.7 | 0.5 | 0.0 | 24.1 | 4.9 | 0.7 | 16.5 | 27.1 | 0.8 | 6.6 |
| PPT 10° C. | 0.2 | 7.1 | 0.5 | 0.0 | 25.1 | 5.5 | 0.8 | 15.5 | 29.0 | 0.0 | 8.1 |
| PPT 4° C. | 0.2 | 2.8 | 0.3 | 1.6 | 25.1 | 7.1 | 0.9 | 7.3 | 34.0 | 0.6 | 8.4 |
| SUP 4° C. | 0.1 | 0.6 | 0.2 | 0.9 | 11.8 | 11.0 | 1.4 | 2.9 | 35.5 | 0.7 | 7.9 |
| TAG fractions | | | | | | | | | | | |
| PPT 20° C. | 0.1 | 15.1 | 0.2 | 0.0 | 22.8 | 0.4 | 0.0 | 30.8 | 26.8 | 0.5 | 2.2 |
| PPT 15° C. | 0.0 | 1.8 | 0.0 | 0.0 | 17.0 | 0.1 | 0.0 | 51.6 | 26.6 | 0.3 | 0.8 |
| PPT 12.5° C. | 0.0 | 1.2 | 0.0 | 0.0 | 19.7 | 0.1 | 0.0 | 44.4 | 32.6 | 0.0 | 0.6 |
| PPT 10° C. | 0.0 | 2.0 | 0.0 | 0.0 | 25.7 | 0.1 | 0.0 | 38.4 | 31.9 | 0.0 | 0.8 |
| PPT 4° C. | 0.0 | 0.8 | 0.1 | 0.0 | 34.3 | 0.1 | 0.0 | 30.1 | 31.8 | 0.3 | 1.6 |
| SUP 4° C. | 0.0 | 1.2 | 0.2 | 0.1 | 34.7 | 0.8 | 0.0 | 20.0 | 32.0 | 1.2 | 8.1 |

| | C18:3 | C18:3 | | | C20:3 | | | | | | |
|---|---|---|---|---|---|---|---|---|---|---|---|
| | ω6 | Δ3 | | | ω6 | | | | | | % of |
| | GLA | ALA | C20:0 | C20:1 | DGLA | ARA | C22:0 | C22:1 | C24:0 | mg | total |
| Polar lipid | | | | | | | | | | | |
| fractions | | | | | | | | | | | |
| PPT 20° C. | 0.8 | 0.0 | 0.0 | 0.1 | 0.2 | 11.4 | 0.0 | 0.0 | 0.1 | 0.27 | 30.7 |
| PPT 15° C. | 1.2 | 0.0 | 0.0 | 0.0 | 0.0 | 12.7 | 0.0 | 0.7 | 0.0 | 0.02 | 1.8 |
| PPT 12.5° C. | 0.8 | 0.0 | 0.0 | 0.7 | 0.0 | 6.6 | 0.0 | 0.0 | 0.0 | 0.02 | 1.8 |
| PPT 10° C. | 0.7 | 0.0 | 0.0 | 0.5 | 0.0 | 5.8 | 0.0 | 1.1 | 0.0 | 0.00 | 0.4 |
| PPT 4° C. | 0.7 | 0.0 | 0.3 | 0.3 | 0.2 | 9.6 | 0.2 | 0.4 | 0.0 | 0.04 | 4.4 |
| SUP 4° C. | 1.4 | 0.2 | 0.1 | 0.1 | 0.3 | 24.8 | 0.1 | 0.0 | 0.1 | 0.54 | 60.9 |
| TAG fractions | | | | | | | | | | | |
| PPT 20° C. | 0.0 | 0.1 | 0.8 | 0.0 | 0.0 | 0.0 | 0.1 | 0.0 | 0.1 | 0.26 | 0.4 |
| PPT 15° C. | 0.0 | 0.0 | 1.4 | 0.0 | 0.0 | 0.0 | 0.2 | 0.0 | 0.1 | 4.4 | 6.4 |
| PPT 12.5° C. | 0.0 | 0.0 | 1.2 | 0.0 | 0.0 | 0.0 | 0.1 | 0.0 | 0.1 | 28.4 | 41.0 |
| PPT 10° C. | 0.1 | 0.0 | 0.9 | 0.0 | 0.0 | 0.0 | 0.1 | 0.0 | 0.0 | 2.0 | 2.9 |
| PPT 4° C. | 0.0 | 0.1 | 0.6 | 0.0 | 0.0 | 0.0 | 0.1 | 0.0 | 0.1 | 15.2 | 21.9 |
| SUP 4° C. | 0.0 | 0.5 | 0.7 | 0.1 | 0.0 | 0.0 | 0.1 | 0.0 | 0.1 | 19.0 | 27.4 |

Y. lipolytica Δ12 desaturase (SEQ ID NO: 1) is a protein of 419 amino acid residues. The protein contains three histidine motifs, typical for fatty acid desaturases, at amino acid positions 121-125, 157-161 and 343-347. These histidine motifs are highly conserved among all FAD2 homologs. When the Y. lipolytica Δ12 desaturase was compared to other microbial desaturases, the protein was related but phylogenetically distinct from the Δ12- and Δ15-desaturases of the ascomycetous yeasts L. kluyveri (Accession No. Q765N3), K. pastoris (Q5BU99), K. lactis (Q6CKY7), C. albicans (Q59WT3), C. parapsilosis (C3W956) and O. polymorpha (E5DCJ6) (Tezaki et al., 2017). Multiple sequence alignment of FAD2 homologs revealed that the fungal homologs exhibited at least 46% sequence homology within the fatty acid desaturase domain (PF00487), which spanned the amino acid region from positions 102-375 of SEQ ID NO:1.

Genetic Constructs for Introducing a FAD2 Gene Deletion into Y. lipolytica

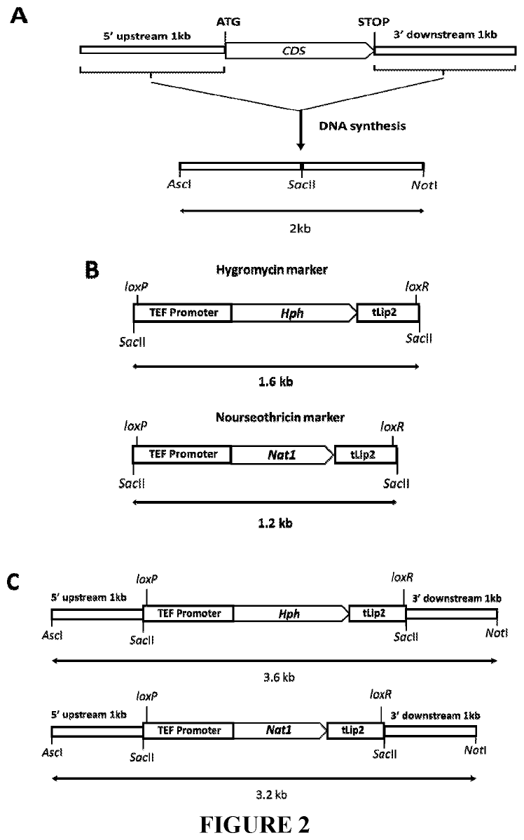

The present inventors wished to reduce the ability of the endogenous Δ12 desaturase to convert oleic acid to LA and so reduce the amount of PUFA in Y. lipolytica. To delete the protein coding sequence of the FAD2 gene from the Y. lipolytica genome and thereby inactivate the gene completely, providing a null mutation, the general strategy of Fickers et al. (2003) was used, with modifications for use of different restriction enzyme sites, as follows. A schematic representation of the strategy is shown in FIG. 2. The strategy involved construction of a genetic cassette which had the protein coding region of the gene of interest replaced with a selectable marker gene, flanked by 5′ upstream and 3′ downstream sequences which provided for integration of the genetic cassette by recombination into the endogenous gene, so deleting the protein coding region. The genetic constructs used a selectable marker gene which provided resistance to an antibiotic, either hygromycin or nourseothricin, providing selection alternatives as appropriate for the context. The 5′ upstream and 3′ downstream sequences of 1,000 base pairs each were homologous to the target gene to allow for recombination in each region.

The nucleotide sequence of the FAD2 gene of Y. lipolytica strain W29 and its upstream and downstream sequences were extracted from the KEGG Yarrowia database (www.genome.jp/kegg-bin/show_organism?org=yli), as gene YALI0B10153p, Accession No. XP_500707, using the known amino acid sequence as a query. SEQ ID NO:2 herein provides the nucleotide sequence of the FAD2 gene including 1,000 nucleotides upstream of the protein coding sequence, presumably including the FAD2 promoter, followed by the protein coding sequence and 1,000 nucleotides downstream of the protein coding sequence.

A DNA fragment corresponding to the 5′ upstream sequence of 1,000 base pairs joined through a SacII restriction enzyme site to the 3′ downstream sequence of 1,000 base pairs was synthesised by GeneArt (Thermofisher, USA). The DNA fragment had flanking AscI and NotI restriction sites which were used to insert the fragment into a pMK vector, forming the construct pAT042. The nucleotide sequence of the cloned insert was confirmed. By joining the 5′ upstream sequence to the 3′ downstream sequence without an intervening FAD2 protein coding sequence, this arrangement effectively deleted the FAD2 protein coding sequence of 1,260 base pairs (Afad2).

### Selectable Marker Genes

Genetic cassettes for providing resistance to the antibiotics hygromycin (Hyg) or nourseothricin (Nat1) as described by Larroude et al. (2018) were obtained from Addgene (Watertown, MA. USA), encoding the Hyg (SEQ ID NO:4) and Nat1 (SEQ ID NO:6) polypeptides, respectively. Each of the genes was under the control of a promoter from a translation elongation factor-la (pTEF) gene from Y. lipolytica (SEQ ID NO:7; Muller et al., 1998) which is a strong, constitutive promoter in Y. lipolytica, and a Y. lipolytica strain U6 lipase 2 gene polyadenylation region/transcription terminator (tLip2; Darvishi et al., 2011; Accession No. HM486900). The nucleotide sequences are provided as SEQ ID NO:3 and SEQ ID NO:5. The DNA fragments including the Hyg and Nat1 transcriptional units from GGE367 and GGE368 were modified by PCR to add a SacII restriction site at each end by using oligonucleotide primers at003 and at004 (Table 13). The modified DNA fragments were ligated into the vector pCR Zero Blunt TOPO (Thermofisher USA; Cat. No. 450245) and the nucleotide sequences of the cloned fragments confirmed. The resultant genetic constructs containing the Hyg and Nat1 sequences were designated pAT121 and pAT122, respectively (Table 14). In a pair of analogous modifications, the DNA fragments including the Hyg and Nat1 transcriptional units were modified by using primers at229 and at230 (Table 13) to add flanking AsiSI sites, generating pAT123 and pAT124.

In the process used to add the flanking SacII restriction sites, the design of the primers provided for the retention of the loxP site at the 5′ end of the TEF promoter and the loxR site at the 3′ end of the Lip2 terminator (FIG. 2), thus flanking the Hyg and Nat1 resistance gene cassettes. These recombinational sites were retained so that the resistance genes, after integration into the microbial genome, could subsequently be excised by Cre/lox recombination. This design allowed for re-use of the same selectable marker gene in multiple rounds of gene deletions, as described further below.

A sample of DNA of pAT121 was digested with SacI, electrophoresed on an agarose gel, and the fragment with the hygromycin resistance gene purified from the gel using a gel extraction kit (Qiagen, USA, Cat. No. 28704). The DNA fragment was then ligated to pAT042 which had been digested with SacII and treated with calf intestinal alkaline phosphatase (New England Biolabs, USA). The ligation mix was introduced into E. coli DH5a competent cells by a standard transformation method. Kanamycin resistant colonies were selected. DNA was prepared from five colonies and screened by digestion with the restriction enzymes XmaI, AscI and NotI and agarose gel electrophoresis to identify and confirm that the correct insertion of the Hyg resistance cassette had occurred into the SacII site between the 5′ upstream and 3′ downstream FAD2 sequences. The resultant constructs having the Hyg antibiotic resistance gene sequences flanked by the 5′ upstream and 3′ downstream sequences from FAD2 was retained and designated pAT259. An analogous construction using the nourseothricin resistance gene cassette (Nat1) resulted in the generation of a genetic construct designated as pAT260 (Table 14 and FIG. 2).

Introduction of a FAD2 Deletion Construct into Y. lipolytica

To introduce the genetic construct in pAT259 containing the hygromycin resistance gene into Y. lipolytica and identify genetically modified cells from the transformation, the following protocol was followed. Cells of Y. lipolytica strain W29 to be transformed were streaked onto a YPD-agar plate and incubated at 28° C. for 16 h. A loopful of the freshly grown cells was scraped from the agar surface. The cells were washed in 1 mL of TE buffer (10 mM Tris-HCl, 1 mM EDTA, pH 8.0) and pelleted by centrifugation at 15,800 g for 1 min at room temperature. The cells were resuspended in 600 μL of 0.1 M lithium acetate (LiAc) solution and incubated at 28° C. for 1 h to generate competent cells. The cell suspension was then centrifuged at 400 g for 2 min at room temperature and the cells gently resuspended in 60 μL of 0.1 M LiAc solution. 40 μL of the competent cells were transferred to a 2 mL tube and mixed gently with 3 to 10 μL (˜500 to 1,000 ng DNA) of AscI/NotI linearized DNA vector and 3 μL of carrier DNA (5 mg/mL). The mixtures were incubated at 28° C. for 15 min. 350 μL of PEG 4000 solution was added to each transformation and mixed gently. The mixtures were incubated at 28° C. for 1 h, followed by a heat shock at 39° C. for 10 min. 600 L of LiAc 0.1M solution was added and mixed gently.

Each transformation mix was cultured in 5 mL of non-selective medium (YPD) for 24 h to provide for recovery of transformants. The cells were then diluted, plated onto selective YPD medium containing hygromycin (250 μg/mL), and the plates incubated at 28° C. for 2 days to obtain 50-100 colonies per plate. When nourseothricin was used as the selective agent in combination with introduction of the Nat1 gene in an analogous construction, the antibiotic was used at a concentration of 400 μg/mL.

Hygromycin resistant colonies from the Y. lipolytica transformation were screened by PCR for the FAD2 gene insertion using oligonucleotide primers at239 and at240 and colonies that were positive for the gene deletion/insertion of Hyg were selected. To test the phenotype and confirm the FAD2 deletion mutations, four hygromycin resistant colonies were grown in YPD medium at 28° C. and the fatty acid composition of the total lipid extracted from cells determined by GC quantitation of FAME. The results (Table 15) showed that the lipids from all four transformants lacked LA, confirming that the FAD2 gene was inactivated in each isolate with a concomitant increase in the level of oleic acid to about 76% of the total fatty acid content. Strains which were wild-type for FAD2 and included as controls were grown under the same conditions. They had lipid with about 18% LA and 56% oleic acid. The observed fatty acid composition of the lipid in the fad2 mutants was similar to that reported in WO2004/104167, at 74% oleic acid and no detectable LA.

To confirm the lack of PUFA other than LA in the strains, the Y. lipolytica transformants were grown for up to three days in YPD medium. No polyunsaturated fatty acids other than, in some samples, a trace amount of LA (0.1%) were observed in either the polar lipid or the TAG from the cells (Table 16).

TABLE 13
Primer sequences used to create deletional mutations in endogenous genes.

| | | | SEQ |
|---|---|---|---|
| | | | ID |
| Primer | Gene/KO | Sequence | NO |
| at063 | SacII-Hyg/Nos_F | GGGCCGCGGTAGGGATAACAGGGTAATGATAAC | 8 |
| at064 | SacII-Hyg/Nos_R | CCCCCGCGGATTACCCTGTTATCCCTACTCGC | 9 |
| at213 | LRO_DelCas5F | GGGGGCGCGCCGAGTCGACTTTCCGATACAGAAG | 10 |
| at214 | LRO_DelCas5R | CCCGCGATCGCTGATGGTTGTGATCAACCGGAAA | 11 |
| at215 | LRO_DelCas3F | GGGGCGATCGCAGAGTCCGTTTTGTAGAGTAATATG | 12 |
| at216 | LRO_DelCas3R | CCCGCGGCCGCACGGACCTCGCAAGTTGCAAAAAG | 13 |
| at217 | DGA1_DelCas5F | GGGGGCGCGCCATGCTGCGGGCGGATCCTGG | 14 |
| at218 | DGA1_DelCas5R | CCCGCGATCGCAGCTTTTGTTTTGTGTGACTTGTC | 15 |
| at219 | DGA1_DelCas3F | GGGGCGATCGCGGAAAACTGCCTGGGTTAGGC | 16 |
| at220 | DGA1_DelCas3R | CCCGCGGCCGCTCTGATGGCCTGGAGCGAGTT | 17 |
| at221 | DGA2_DelCas5F | GGGGGCGCGCCTGGGAGTGTATTTGGAAAATGACT | 18 |
| at222 | DGA2_DelCas5R | CCCGCGATCGCTTTGCGGGCGGTACGGGTAC | 19 |
| at223 | DGA2_DelCas3F | GGGGCGATCGCCATAACACTCATCAGTAGCCTTTAC | 20 |
| at224 | DGA2_DelCas3R | CCCGCGGCCGCTCTTGTAATTCCATAGATAATATATACG | 21 |
| at225 | ARE1_DelCas5F | GGGGGCGCGCCGCCGACAATCTCTCTTCCTCATT | 22 |
| at226 | ARE1_DelCas5R | CCCGCGATCGCTGTGTGTGCGGAGAGTGTCTTG | 23 |
| at227 | ARE1_DelCas3F | GGGGCGATCGCGCACAGTCGCTTCACCACTTG | 24 |
| at228 | ARE1_DelCas3R | CCCGCGGCCGCTAAAACAATGACAAATACAACTCTAG | 25 |
| at229 | AsiSI-Hyg/Nos_F | GGGGCGATCGCTAGGGATAACAGGGTAATGATAAC | 26 |
| at230 | AsiSI-Hyg/Nos_R | CCCGCGATCGCATTACCCTGTTATCCCTACTCGC | 27 |
| at239 | Fad2-gene_F | GTGTCCGAGCCCGTCTACG | 28 |
| at240 | Fad2-gene_R | ACATGGTGATACCAATACCGAC | 29 |
| at241 | ARE1KOScreen_F1 | GATGCAAATGACGCACGGCC | 30 |
| at242 | ARE1KOScreen_F2 | CCAAACGACACTCGATAGTG | 31 |
| at243 | ARE1KOScreen_R1 | TGCTTTGCGTGGGAGAGCTC | 32 |
| at244 | ARE1KOScreen_R2 | CAGTGGTTGACATTCTATAGGCAC | 33 |
| at245 | DGA1KOScreen_F1 | CAGGCAACAGACAAGTCACAC | 34 |
| at246 | DGA1KOScreen_F2 | CATCTGGCTCCAACGGCCAC | 35 |
| at247 | DGA1KOScreen_R1 | CAGCTGTGGGTCGTTTTCGC | 36 |
| at248 | DGA1KOScreen_R2 | GCTATTTGCCTAACCCAGGC | 37 |
| at249 | DGA2KOScreen_F1 | CATAGCTACACCCAAAGTCGC | 38 |
| at250 | DGA2KOScreen_F2 | CGCTCAGCGGACTGACCATG | 39 |
| at251 | DGA2KOScreen_R1 | TCTTGGGGTACACGGGCTGG | 40 |
| at252 | DGA2KOScreen_R2 | CTGTAAAGGCTACTGATGAGTG | 41 |
| at257 | LRO1KOScreen_F1 | CATGACTAATCATTCACGCGAC | 42 |
| at258 | LRO1KOScreen_F2 | CGTGGTCGGTGTGATAGCCG | 43 |
| at259 | LRO1KOScreen_R1 | GTTCGCAGCAGATCGGCTCG | 44 |
| at260 | LRO1KOScreen_R2 | CGAACGCCCTTCTCCATCAGTG | 45 |
| at270 | URA3KOScrnF1 | CGTTGGAGGCTGTGGGTCTG | 46 |
| at271 | URA3KOScrnF2 | AGTGCTCAAGCTCGTGGCAG | 47 |
| at272 | URA3KOScrnR1 | GATCTCGGTTCTGGCCGTAC | 48 |
| at273 | URA3KOScrnR2 | GCCTCCAGGAAGTCCATGGG | 49 |

TABLE 14
Genetic constructs made for gene deletions.

| Construct | Description |
|---|---|
| pAT042 | YlFAD2 5′ upstream and 3′ downstream joined |
| pAT069 | YlURA3 5′ upstream and 3′ downstream joined, in pMK-T |
| pAT070 | YlURA3 5′ upstream and 3′ downstream joined, in pMK-RQ |
| pAT121 | Hyg resistance gene with added SacII sites (from GGE367) |
| pAT122 | Nat1 resistance gene with added SacII sites (from GGE368) |
| pAT123 | Hyg resistance gene with added AsiSI sites (from GGE367) |
| pAT124 | Nat1 resistance gene with added AsiSI sites (from GGE368) |
| pAT251 | ARE1 5′ upstream and 3′ downstream joined, with AsiSI site |
| pAT253 | DGA1 5′ upstream and 3′ downstream joined, with AsiSI site |
| pAT254 | DGA2 5′ upstream and 3′ downstream joined, with AsiSI site |
| pAT256 | LRO1 5′ upstream and 3′ downstream joined, with AsiSI site |
| pAT257 | URA3-Hyg-Del Cassette (pAT070 + pAT121) |
| pAT258 | URA3-Nos-Del Cassette (pAT070 + pAT122) |
| pAT259 | FAD2-Hyg-Del Cassette (pAT042 + pAT121) |
| pAT260 | FAD2-Nat1-Del Cassette (pAT042 + pAT122) |
| pAT261 | ARE1 Hyg Del Cassette (pAT251 + pAT257) |
| pAT262 | ARE1 Nat1 Del Cassette (pAT251 + pAT258) |
| pAT265 | DGA1 Hyg Del Cassette (pAT253 + pAT257) |
| pAT266 | DGA1 Nos Del Cassette (pAT253 + pAT258) |
| pAT267 | DGA2 Hyg Del Cassette (pAT254 + pAT257) |
| pAT268 | DGA2 Nat1 Del Cassette (pAT254 + pAT258) |
| pAT271 | LRO1 Hyg Del Cassette (pAT256 + pAT257) |
| pAT272 | LRO1 Nat1 Del Cassette (pAT256 + pAT258) |

TABLE 15
Fatty acid composition of lipid from fad2 deletion
mutants of Y. lipolytica, showing only the
main 5 fatty acids present in wild-type strains.

| | | | | | C18:2Δ9, |
|---|---|---|---|---|---|
| | C16:0 | C16:1 | C18:0 | C18:1Δ9 | 12 |
| FAD2-1 | 10.1 | 10.2 | 3.3 | 76.4 | 0.0 |
| FAD2-5 | 10.2 | 9.7 | 3.7 | 76.4 | 0.0 |
| FAD2-6 | 10.3 | 9.5 | 3.8 | 76.3 | 0.0 |
| FAD2-9 | 10.4 | 9.4 | 3.5 | 76.8 | 0.0 |
| Average for fad2 | 10.2 | 9.7 | 3.6 | 76.5 | 0.0 |
| deletion | | | | | |
| KO27-1 | 12.4 | 9.1 | 3.9 | 55.8 | 18.8 |
| KO27-2 | 12.4 | 8.9 | 4.2 | 57.1 | 17.3 |
| KO27-3 | 12.4 | 9.2 | 3.7 | 55.5 | 19.2 |
| Average wild-type | 12.4 | 9.1 | 3.9 | 56.1 | 18.4 |
| FAD2 | | | | | |

TABLE 16
Fatty acid composition of polar lipid and TAG fractions from Y. lipolytica
fad2 mutant during culturing in YPD medium for up to 3 days.

| | | | | | | | | | |
|---|---|---|---|---|---|---|---|---|---|
| | | | | C16:1 | C16:1 | | | C18:1 | C18:1 |
| Time | C14:0 | C15:0 | C16:0 | Δ7 | Δ9 | C17:1 | C18:0 | Δ9 | Δ11 |

Polar lipid

| | | | | | | | | | |
|---|---|---|---|---|---|---|---|---|---|
| 24 h | 0.1 | 0.4 | 9.7 | 2.3 | 12.5 | 1.3 | 0.8 | 72.0 | 0.8 |
| 48 h | 0.1 | 0.3 | 8.0 | 4.9 | 12.4 | 1.9 | 0.3 | 71.5 | 0.6 |
| 72 h | 0.0 | 0.2 | 6.0 | 3.6 | 11.5 | 3.3 | 0.2 | 74.5 | 0.5 |

TAG

| 24 h | 0.2 | 0.7 | 14.6 | 1.2 | 9.0 | 0.8 | 15.2 | 48.7 | 1.1 |
|---|---|---|---|---|---|---|---|---|---|
| 48 h | 0.1 | 0.5 | 14.6 | 2.4 | 8.7 | 0.9 | 11.5 | 53.2 | 1.0 |
| 72 h | 0.1 | 0.4 | 13.1 | 1.8 | 6.9 | 1.5 | 18.0 | 52.0 | 0.8 |
| | C18:2 | | C20:1 | C20:2 | C20:3 | C20:4 | | | Lipid % |
| | Δ9, 12 | C20:0 | Δ11 | Δ11, 14 | ω6 | ω6 | C22:0 | C24:0 | per cdw |

Polar lipid

| | | | | | | | | | |
|---|---|---|---|---|---|---|---|---|---|
| 24 h | 0.0 | 0.0 | 0.0 | 0.0 | 0.0 | 0.0 | 0.0 | 0.1 | 1.8 |
| 48 h | 0.1 | 0.0 | 0.0 | 0.0 | 0.0 | 0.0 | 0.0 | 0.0 | 1.0 |
| 72 h | 0.0 | 0.0 | 0.1 | 0.0 | 0.0 | 0.0 | 0.0 | 0.1 | 1.7 |

TAG

| | | | | | | | | | |
|---|---|---|---|---|---|---|---|---|---|
| 24 h | 0.1 | 0.7 | 0.0 | 0.0 | 0.0 | 0.0 | 0.7 | 7.2 | 0.3 |
| 48 h | 0.1 | 0.5 | 0.1 | 0.0 | 0.0 | 0.0 | 0.6 | 5.8 | 0.4 |
| 72 h | 0.0 | 0.6 | 0.0 | 0.0 | 0.0 | 0.0 | 0.7 | 4.1 | 0.7 |

The TAG samples also lacked C18:3ω6.

### Example 7. Modification of Y. lipolytica to Generate a Uracil Auxotroph

The URA3 gene of Y. lipolytica encodes the enzyme orotidine-5′-phosphate decarboxylase (EC 4.1.1.23; GenBank Accession No. Q12724), with a variant sequence as Accession No. AJ306421.1 (Mauersberger et al., 2001). The enzyme is required in microbes for synthesis of uracil, so that null mutants in the URA3 gene require the addition of uracil in the medium in order to grow. Such auxotrophic mutants have been used with genetic constructs including a functional URA3 gene as a selectable marker gene, selecting for complementation of the URA3 mutation on defined medium lacking uracil (Mauersberger et al., 2001). Therefore, a URA3 gene deletion mutant was made in Y. lipolytica, starting from the wild-type W29 strain. The strategy used was analogous to that for the fad2KO1 mutant (Example 6, FIG. 2).

Genetic Constructs for Introducing a URA3 Gene Deletion into Y. lipolytica

The nucleotide sequences of the upstream and downstream regions, of 1,000 basepairs each, of the URA3 gene of Y. lipolytica strain W29 were extracted from the NCBI database using the sequence from U40564.1 (www.ncbi.nlm.nih.gov/nuccore/U40564.1/) as a query. The chromosome E sequence was chosen with the identity parameter at 100% and the Changed Region option set to positions 3150692-3154401 to provide a wider range of upstream and downstream sequences. The nucleotide sequence of the URA3 gene is provided as SEQ ID NO:51 herein including the 1,000 nucleotides upstream of the protein coding sequence and the 1,000 nucleotides downstream. The amino acid sequence of the encoded orotidine-5′-phosphate decarboxylase polypeptide from Y. lipolytica is provided as SEQ ID NO:50.

A DNA fragment corresponding to the 5′ upstream sequence of 1,000 basepairs joined through a SacII restriction enzyme site to the 3′ downstream sequence of 1,000 base pairs was synthesised by GeneArt (Thermofisher, USA), initially in the vector pMK-T, forming pAT069. The DNA fragment had flanking AscI and NotI restriction sites which were used to insert the fragment into a pMK-RQ vector, forming the construct pAT070 (Table 14, Example 6). The nucleotide sequence of the cloned insert was confirmed. By joining the 5′ upstream sequence to the 3′ downstream sequence without an intervening URA3 protein coding sequence, this arrangement effectively deleted the URA3 protein coding sequence of 861 basepairs (AURA3).

The DNAs of pAT121 including the hygromycin resistance gene and pAT122 including the nourseothricin resistance gene (Example 6) were digested with SacI and the fragments spanning the genes purified using a gel extraction kit (Qiagen, USA). The DNA fragments were separately ligated with pAT070 which had been digested with SacII and treated with calf intestinal alkaline phosphatase. The ligation mixes were transformed into E. coli DH5a competent cells. DNA was prepared from five colonies for each ligation and DNA samples from the colonies were screened by digestion with the several restriction enzymes and agarose gel electrophoresis to identify and confirm that the correct insertions had occurred between the 5′ upstream and 3′ downstream sequences. The resultant constructs having the Hyg or Nat1 antibiotic resistance gene sequences flanked by the 5′ upstream and 3′ downstream sequences from URA3 were designated pAT257 and pAT258, respectively (Table 14).

### Introduction of URA3 Deletion Constructs

To introduce the genetic construct pAT257 containing the hygromycin resistance gene into Y. lipolytica and identify genetically modified Ura auxotrophic cells from the transformation, the transformation protocol described in Example 6 was followed. Transformed cells were selected on YPD plates containing 250 μg/mL hygromycin. Antibiotic resistant colonies were screened by PCR for the URA3 gene insertion and for uracil auxotrophy. For uracil auxotrophy, the hygromycin resistant colonies were screened on YPD plates and SD-Ura plates, each also containing hygromycin. Colonies that grew on both the YPD and SD-Ura plates were discarded as negatives for the gene deletion, while the colonies that grew on YPD but not on SD-Ura plates were selected as having ura gene deletions. The positive colonies were screened by PCR using primers at270 and at272 (Table 13, Example 6) with the Phire DNA PCR kit (ThermoFisher). Initial denaturation was at 98° C. for 5 min; followed by 40 cycles of 98° C. for 5 sec, 60° C. for 5 sec and 72° C. for 20 sec per 1 kb, with a final extension of 72° C. for 4 min. Several positives colonies were retested using Taq polymerase with ThermoPol buffer (NEB Biolabs, USA Cat #M0267) to confirm the validity of the gene deletion.

One of the transformed cell lines was retained as the Y. lipolytica ura3 deletion mutant and designated Y. lipolytica strain ura3KO27. This strain was used for introduction of various single-gene (in addition to Ura3 gene) and multi-gene genetic constructs, allowing for selection of the Ura⁺ phenotype.

### Generation of Double Deletion Mutant Fad2-Ura3

The fad2KO1 mutant of Y. lipolytica described in Example 6 was modified in a second round of transformation to introduce a URA3 gene deletion. This used the transformation protocol described above except that pAT258 was used, having the Nat1 selectable marker gene providing resistance to nourseothricin on YPD medium containing the antibiotic. Colonies that grew on plates containing nourseothricin at a concentration of 400 μg/mL were confirmed as having the ura3 deletion mutation: antibiotic resistant colonies were screened by PCR for the URA3 gene insertion and for uracil auxotrophy, as described above for the ura3KO27 strain. One double mutant strain was retained and designated fad2KO1-ura3KO27. This strain was used for introduction of various single-gene and multi-gene genetic constructs, allowing for selection of the Ura⁺ phenotype in a fad2 mutant background.

### Example 8. Modification of Microbes to Reduce Triacylglycerol Synthesis—Single Gene Mutants

Triacylglycerol (TAG) synthesis in yeasts such as S. cerevisiae and Y. lipolytica occurs by the activity of a suite of enzymes, mostly through the Kennedy pathway, where free fatty acids are firstly linked to coenzyme A (CoA) to produce acyl-CoA molecules. The acyl groups from three acyl-CoAs are then esterified in a step-wise fashion to a glycerol backbone to synthesize TAG. In the first step, glycerol-3-phosphate (G3P) is acylated by a glycerol-3-phosphate acyltransferase (GPAT; EC 2.3.1.15), encoded by the SCT and GPT2 genes in S. cerevisiae and the YALI0C00209 g gene in Y. lipolytica, to produce lysophosphatidic acid (LPA). LPA is then acylated by lysophosphatidic acid acyltransferase (LPAAT; EC 2.3.1.51; also referred to as 1-acyl-sn-G3P acyltransferase), encoded by the SLC1 gene in S. cerevisiae and the YALI0E18964 g gene in Y. lipolytica, to produce phosphatidic acid (PA). This is followed by dephosphorylation of PA by the enzyme phosphatidic acid phosphohydrolase (PAP) to produce diacylglycerol (DAG). In the final step, DAG is acylated by either one of two diacylglycerol acyltransferases (EC 2.3.1.20), DGA1 or DGA2, with acyl-CoA as the acyl donor. DGA1 is encoded by the DGA1 gene in S. cerevisiae and the YALI0E32769 g gene in Y. lipolytica. TAG can also be synthesized from DAG by phospholipid:diacylglycerol acyltransferase (PDAT, also known as phospholipid:1,2-diacyl-sn-glycerol O-acyltransferase; EC 2.3.1.158), encoded by the LRO1 gene in S. cerevisiae and the YALI0E16797 g gene in Y. lipolytica, which uses a glycerophospholipid as the acyl donor to produce the TAG. Two different acyl-CoA:sterol acyltransferases (ASAT, EC 2.3.1.26) can also synthesize TAG in S. cerevisiae, encoded by the ARE1 and ARE2 genes, and a single ARE gene, YALI0F06578 g, in Y. lipolytica.

Y. lipolytica is considered to be an oleaginous yeast since it can produce more than 20% by weight of lipid (dry cell weight), in some strains up to at least 30% TAG under growth conditions with limited nitrogen. With certain genetic modifications, Y. lipolytica strains can be engineered to produce up to 77% lipid by weight or even more. There are numerous other known oleaginous fungi including other yeasts. In contrast, most strains of S. cerevisiae do not make copious TAG and are not considered to oleaginous yeasts, with the exception of a few strains such as D5A (He et al., 2018).

The present inventors considered that lipids containing increased amounts of LC and VLC saturated fatty acids could be produced in yeast strains that were genetically modified to produce more TAG and/or TAG having modified fatty acid composition. Experiments were therefore designed to first of all inactivate TAG synthesis genes including the DGA1, DGA2, LRO1 and ARE1 genes in Y. lipolytica and test their function.

Genetic Constructs for Introducing a DGA1 Gene Deletion into Y. lipolytica

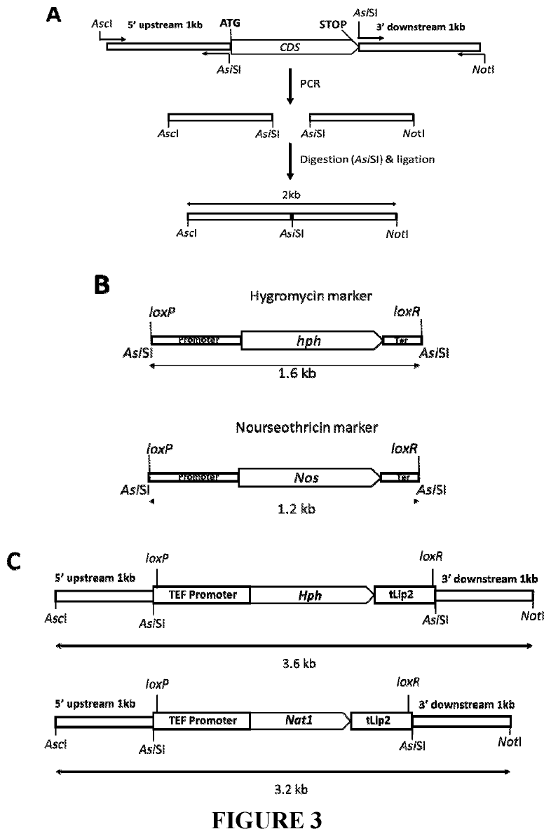

To delete the protein coding sequence of the DGA1 gene and other TAG synthesis genes from the Y. lipolytica genome, thereby providing null mutations, the general strategy described in FIG. 2 for FAD2 was modified in several aspects. A schematic representation of the modified strategy is shown in FIG. 3. As before, the genetic cassette for introducing the gene deletions had the protein coding region of the gene of interest replaced with a selectable marker gene, flanked by 5′ upstream and 3′ downstream sequences which provided for integration of the genetic cassette by recombination into the endogenous gene. This time, however, the 5′ upstream and 3′ downstream sequences of 1,000 basepairs were produced by PCR in-house. Also, the primers used in the amplifications and the selectable marker genes had AsiSI restriction enzyme sites rather than SacII sites.

The nucleotide sequence of the DGA1 gene of Y. lipolytica strain W29 and its upstream and downstream sequences were extracted from the KEGG Yarrowia database (www.genome.jp/kegg-bin/show_organism?org=yli) using the published YALI gene identifier, as gene YALI0E32769p, nucleotides 3885857 to 3889401 of chromosome E, Accession No. CR382131.1. The nucleotide sequence of the DGA1 gene is provided herein as SEQ ID NO:52 including 1,000 nucleotides upstream of the protein coding sequence followed by the protein coding sequence and 1,000 nucleotides downstream of the protein coding sequence.

The amino acid sequence of the encoded DGA1 polypeptide is provided as SEQ ID NO:53. Y. lipolytica DGA1 is a protein of 514 amino acid residues and is a homolog of animal and plant DGAT2 enzymes, so is considered to be a DGAT2 polypeptide.. These are all members of the MBOAT protein family (Wang et al., 2013).

The 5′ upstream and 3′ downstream regions adjacent to the DGA1 protein coding region were amplified from genomic DNA from Y. lipolytica strain W29. Each amplification reaction used Taq DNA Polymerase with ThermoPol Buffer and a pair of oligonucleotide primers (Table 13 of Example 6). By this means, the 5′ upstream fragment was adapted by adding restriction enzyme sites for AscI at its 5′ end and AsiSI at its 3′ end. Similarly, the 3′ downstream fragment was adapted by adding restriction enzyme sites for AsiSI at its 5′ end and NotI at its 3′ end. (Phusion High Fidelity DNA polymerase, Thermofisher, US) as per manufacturer instructions. The amplified DNA fragments were digested with AsiSI and ligated with T4 DNA Ligase using standard protocols and inserted into vector into the vector pCR Zero Blunt TOPO, forming pAT253 (Table 14 of Example 6). The nucleotide sequence of the cloned insert was confirmed.

The DNAs of pAT123 including the hygromycin resistance gene and pAT124 including the nourseothricin resistance gene (Example 6) were digested with AsiSI and the fragments spanning the genes purified using a gel extraction kit (Qiagen, USA). The DNA fragments were separately ligated with pAT253 DNA which had been digested with AsiSI and treated with calf intestinal alkaline phosphatase. The ligation mixes were transformed into E. coli DH5a competent cells. DNA was prepared from at least five colonies for each ligation and DNA samples from the colonies were screened by digestion with restriction enzymes and agarose gel electrophoresis to identify and confirm that the correct insertions had occurred between the 5′ upstream and 3′ downstream sequences. The resultant constructs having the Hyg or Nat1 antibiotic resistance gene sequences flanked by the 5′ upstream and 3′ downstream sequences from DGA1 were designated pAT265 and pAT266, respectively (Table 14 of Example 6).

Introduction of DGA1 Deletion Constructs into Y. lipolytica

To introduce the genetic constructs pAT265 containing the Hyg resistance gene and pAT266 containing the Nat1 resistance gene to replace the DGA1 protein coding region in Y. lipolytica and identify genetically modified Δdga1 cells from the transformation, the transformation protocol described in Example 6 was followed. Transformed cells were selected on YPD plates containing 250 μg/ml hygromycin or 400 μg/ml nourseothricin, according to the selectable marker gene. Antibiotic resistant colonies were screened by PCR for the DGA1 gene insertion. One oligonucleotide primer (at245) located in the 5′ upstream region and a second primer (at247) located within the DGA1 protein coding region were used in PCR reactions to confirm the deletion mutation had been introduced into the genomic DNA of antibiotic resistant colonies. The PCR reaction was performed using Taq DNA polymerase with ThermoPol buffer (NEB, USA) under standard conditions. Lack of an amplification product indicated the presence of the deletion mutation. Genomic DNA from W29 was used in parallel as a positive control for the PCR. A second PCR test using oligonucleotide primers at245 and at248, the latter located in the 3′ region of DGA1, also confirmed the presence of the deletion/insertion mutation, producing a 1.3 kb amplification product in presence of the deletion and a 1.6 kb product in the wild-type, unmutated DGA1. Primer pair at246 and at248 was also used. The absence of the DGA1 protein coding region was confirmed in 7 of 10 colonies tested for the Nat1 gene.

One of the transformed cell lines from each of the transformations was selected and retained as Y. lipolytica dga1 deletion mutants and designated strains dga1KO1(Hyg) and dga1KO1(Nat1).

The dga1KO1 strains were compared to the corresponding wild-type strain by growth in a high glucose/low nitrogen medium that induces TAG synthesis, to determine the reduction in TAG synthesis ability. A reduction of 50% in the level of TAG is observed in the dga1KO1 mutants after 96 h culturing at 29° C. in the latter medium. Surprisingly, it was observed (see Example 11) that the amount of VLC-SFA, particularly C24:0, was substantially reduced in the dga1 mutant. Indeed, the level of C24:0 in TAG as a percentage of the total fatty acid content was reduced by 83%. The level of C18:0 was also substantially reduced, and the percentage of the total saturated fatty acids decreased from about 55% in wild-type strain W29 to about 35% in the dga1 mutant. It was concluded that the DGA1 protein was responsible for most of the incorporation of VLC-SFA, particularly C24:0, and also C18:0 into TAG in Y. lipolytica. The dga1KO1 strain is also grown in a rich medium such as YPD.

Genetic constructs for introducing a DGA2 gene deletion into Y. lipolytica

To delete the protein coding sequence of the DGA2 gene from the Y. lipolytica genome, the same strategy was used as for the DGA1 deletion (FIG. 3). The nucleotide sequence of the DGA2 gene of Y. lipolytica strain W29 and its upstream and downstream sequences were extracted from the KEGG Yarrowia database (www.genome.jp/kegg-bin/show_organism?org=yli), using the published YALI gene identifier, as gene YALI0B10153p, Accession No. XP_500707. The nucleotide sequence of the DGA2 gene is provided as SEQ ID NO:54 including 1,000 nucleotides upstream of the protein coding sequence followed by the protein coding sequence and 1,000 nucleotides downstream of the protein coding sequence.

The amino acid sequence of the encoded DGA2 polypeptide is provided as SEQ ID NO:55. Y. lipolytica DGA2 is a protein of 526 amino acid residues. The protein is a member of the membrane-bound O-acyltransferase family-domain-containing (MBOAT) protein with multiple membrane spanning regions, typically 8-10 such regions, that transfer acyl groups to substrates in membranes, in this case to DAG. The Y. lipolytica DGA2 protein is more closely related to the DGAT1 proteins of plants and animals and is phylogenetically distinct from the DGAT2s of the ascomycetous yeasts L. kluyveri, K. pastoris, K. lactis, C. albicans, C. parapsilosis and O. polymorpha. It is therefore considered to be a DGAT1. The amino acid sequence of DGA2 polypeptide (SEQ ID NO:55) does not show significant identity with that of DGA1 polypeptide (SEQ ID NO:53) when compared by BLAST program.

The 5′ upstream and 3′ downstream regions were amplified from genomic DNA from Y. lipolytica strain W29 (FIG. 3). Each amplification reaction used Phusion high fidelity DNA polymerase (NEB, USA) and a pair of oligonucleotide primers (Table 13). As for the amplifications for DGA1, the 5′ upstream fragment had a restriction enzyme site for AscI at its 5′ end and one for AsiSI at its 3′ end. Similarly, the 3′ downstream fragment had a site for AsiSI at its 5′ end and one for NotI at its 3′ end. The amplified DNA fragments were digested with AsiSI, ligated with T4 DNA Ligase, and inserted into the vector pCR Zero Blunt TOPO, forming pAT254 (Table 14 of Example 6). The nucleotide sequence of the cloned insert was confirmed.

The DNAs of pAT123 including the hygromycin resistance gene and pAT124 including the nourseothricin resistance gene (Example 6) were digested with AsiSI and the fragments spanning the genes purified using a gel extraction kit. These were ligated into pAT254 which had been digested with AsiSI and the ligation mixes introduced into E. coli DH5a competent cells. DNA was prepared from five colonies for each ligation and screened by digestion with restriction enzymes. Agarose gel electrophoresis identified the correct constructs and confirmed that the intended insertions had occurred between the 5′ upstream and 3′ downstream sequences. The resultant constructs having the Hyg or Nat1 antibiotic resistance gene sequences flanked by the 5′ upstream and 3′ downstream sequences from DGA2 were designated pAT267 and pAT268, respectively (Table 14 of Example 6).

Introduction of DGA2 Deletion Constructs into Y. lipolytica

To introduce the genetic construct pAT267 containing the hygromycin resistance gene into Y. lipolytica, the transformation protocol described in Example 6 was followed. Transformed cells were selected on YPD plates containing 250 μg/mL hygromycin. Antibiotic resistant colonies were screened by PCR for the DGA2 gene insertion to identify genetically modified Δdga2 cells from the transformation. The oligonucleotide primer pair at249 and at251, internal to the DGA2 protein coding region, were used in PCR reactions to confirm the deletion mutation had been introduced into the genomic DNA of hygromycin resistant colonies. The PCR reaction was performed using Taq DNA polymerase with ThermoPol buffer (NEB, USA) under standard conditions. Lack of an amplification product indicated the presence of the deletion mutation. Genomic DNA from W29 was used in parallel as a positive control for the PCR. A second PCR test using oligonucleotide primers at250 and at252, the latter located in the 3′ region of DGA2, also confirmed the presence of the deletion/insertion mutation. The absence of the DGA2 protein coding region was confirmed in 5 of 6 colonies selected with the Hyg gene.

One of the transformed cell lines was retained as a Y. lipolytica dga2 deletion mutant and designated Y. lipolytica strain dga2KO1(Hyg).

The strain dga2KO1(Hyg) was compared to its corresponding wild-type strain by growth in a high glucose/low nitrogen medium (DM-Glyc-LowN) that induces TAG synthesis, to determine the reduction in TAG synthesis ability. A reduction in the level of TAG of about 46% was observed in the dga2KO1 mutant compared to the wild-type DGA2 strain. Surprisingly, it was observed (see Example 11) that the amount of C24:0 was increased in the dga2 mutant relative to the wild-type control. Indeed, the level of C24:0 in TAG as a percentage of the total fatty acid content was increased by about 20%. This observation supported the conclusion made above that the DGA1 protein was responsible for inserting most of the C24:0 in TAG in Y. lipolytica, whereas the DGA2 polypeptide was more active on unsaturated fatty acids than SFA. At the same time, the percentage of the total saturated fatty acids decreased from about 55% in wild-type strain W29 to about 43% in the dga2 mutant.

Genetic Constructs for Introducing a LRO1 Gene Deletion into Y. lipolytica

To delete the protein coding sequence of the LRO1 gene from the Y. lipolytica genome, the same strategy was used as for the DGA1 deletion (FIG. 3). The nucleotide sequence of the LRO1 gene of Y. lipolytica strain W29 and its upstream and downstream sequences were extracted from the KEGG Yarrowia database (www.genome.jp/kegg-bin/show_organism?org=yli), using the published YALI gene identifier, as gene YALI0E16797p, in Accession No. CR382131.1. The nucleotide sequence of the LRO1 gene is provided as SEQ ID NO:56 including 1,000 nucleotides upstream of the protein coding sequence followed by the protein coding sequence and 1,000 nucleotides downstream of the protein coding sequence.

The amino acid sequence of the encoded PDAT polypeptide is provided as SEQ ID NO:57. Y. lipolytica protein encoded by the LRO1 gene is a protein of 648 amino acid residues that has PDAT activity.

The 5′ upstream and 3′ downstream regions adjacent to the LRO1 protein coding region were amplified from genomic DNA from Y. lipolytica strain W29 using Phusion high fidelity DNA polymerase (NEB, USA) and a pair of oligonucleotide primers (Table 13 of Example 6). As for the amplifications for DGA1, the 5′ upstream fragment had a restriction enzyme site for AscI at its 5′ end and one for AsiSI at its 3′ end. Similarly, the 3′ downstream fragment had a site for AsiSI at its 5′ end and one for NotI at its 3′ end. The amplified DNA fragments were digested with AsiSI, ligated with T4 DNA Ligase, and inserted into the vector pCR Zero Blunt TOPO, forming pAT256 (Table 14 of Example 6). The nucleotide sequence of the cloned insert was confirmed.

The DNAs of pAT123 including the hygromycin resistance gene and pAT124 including the nourseothricin resistance gene (Example 6) were digested with AsiSI and the fragments spanning the genes purified using a gel extraction kit. These were ligated into pAT256 which had been digested with AsiSI and the ligation mixes introduced into E. coli DH5a competent cells. DNA was prepared from five colonies for each ligation and screened by digestion with restriction enzymes. The correct constructs were identified by agarose gel electrophoresis, confirming that the intended insertions had occurred between the 5′ upstream and 3′ downstream sequences. The resultant constructs having the Hyg or Nat1 antibiotic resistance gene sequences flanked by the 5′ upstream and 3′ downstream sequences from LRO1 were designated pAT271 and pAT272, respectively (Table 14 of Example 6).

Introduction of LRO1 Deletion Constructs into Y. lipolytica

To introduce the genetic construct pAT272 containing the nourseothricin resistance gene into Y. lipolytica, the transformation protocol described in Example 6 was followed. Transformed cells were selected on YPD plates containing 400 μg/mL nourseothricin. Antibiotic resistant colonies were screened by PCR for the LRO1 gene insertion to identify genetically modified Δlro1 cells from the transformation. One oligonucleotide primer (at257) located in the 5′ upstream region and a second primer (at260) located in the 3′ downstream region of LRO1 were used in PCR reactions to confirm the deletion mutation had been introduced into the genomic DNA of antibiotic resistant colonies. The PCR reaction was performed using Taq DNA polymerase with ThermoPol buffer (NEB, USA) under standard conditions. DNA from W29 was used in parallel as a positive control for the PCR. Lack of a 2.2 kb amplification product and the presence of a 1.4 kb product indicated the presence of the deletion mutation. Confirmatory PCR reactions were carried our using primers at258 and at260. The absence of the LRO1 protein coding region was confirmed in four of ten colonies tested.

One oligonucleotide primer (at245) located in the 5′ upstream region and a second primer (at247) located within the LRO1 protein coding region were used in PCR reactions to confirm the deletion mutation had been introduced into the genomic DNA of antibiotic resistant colonies. The PCR reaction was performed using Taq DNA polymerase with ThermoPol buffer (NEB, USA) under standard conditions. Lack of an amplification product indicated the presence of the deletion mutation. Genomic DNA from W29 was used in parallel as a positive control for the PCR. A second PCR test using oligonucleotide primers at245 and at248, the latter located in the 3′ region of LRO1, also confirmed the presence of the deletion/insertion mutation, producing a 1.3 kb amplification product in presence of the deletion and a 1.6 kb product in the wild-type, unmutated DGA1 gene. The absence of the LRO1 protein coding region was confirmed in 4 of 10 colonies tested for the Nat1 gene.

One of the transformed cell lines was retained as a Y. lipolytica lro1 deletion mutant and designated Y. lipolytica strain lro1KO1.

The strain lro1KO1 was compared to its corresponding wild-type strain by growth in a high glucose/low nitrogen medium (DM-Glyc-LowN) that induces TAG synthesis, to determine the reduction in TAG synthesis ability. A reduction in the level of TAG was observed in the lro1KO1 mutant, by about 30%. The percentage of the total saturated fatty acids decreased from about 55% in wild-type strain W29 to about 50% in the lro1 mutant, so less of a change than in the dga1 and dga2 mutants.

Genetic Constructs for Introducing an ARE1 Gene Deletion into Y. lipolytica

The genes ARE1 and ARE2 in fungi, including S. cerevisiae, encode the enzyme acyl-CoA:sterol acyltransferases (ASAT, EC 2.3.1.26) which can also synthesize TAG. Y. lipolytica appears to have a single ARE gene, namely ARE1. To delete the protein coding sequence of the ARE1 gene from the Y. lipolytica genome, the same strategy was used as for the DGA1 deletion (FIG. 3). The nucleotide sequence of the ARE1 gene of Y. lipolytica strain W29 and its upstream and downstream sequences were extracted from the KEGG Yarrowia database (www.genome.jp/kegg-bin/show_organism?org=yli), using the published YALI gene identifier, as gene YALI0F06578 g, in Accession No. CR382131.1. The nucleotide sequence of the ARE1 gene is provided as SEQ ID NO:58 including 1,000 nucleotides upstream of the protein coding sequence followed by the protein coding sequence and 1,000 nucleotides downstream of the protein coding sequence.

The amino acid sequence of the encoded ASAT polypeptide is provided as SEQ ID NO:59. Y. lipolytica ASAT is a protein of 543 amino acid residues.

The 5′ upstream and 3′ downstream regions adjacent to the ASAT protein coding region were amplified from genomic DNA from Y. lipolytica strain W29 (FIG. 3). Each amplification reaction used Phusion high fidelity DNA polymerase (NEB, USA) and a pair of oligonucleotide primers (Table 13 of Example 6). As for the amplifications for DGA1, the 5′ upstream fragment had a restriction enzyme site for AscI at its 5′ end and one for AsiSI at its 3′ end. Similarly, the 3′ downstream fragment had a site for AsiSI at its 5′ end and one for NotI at its 3′ end. The amplified DNA fragments were digested with AsiSI, ligated with T4 DNA Ligase, and inserted into the vector pCR Zero Blunt TOPO, forming pAT251 (Table 14 of Example 6). The nucleotide sequence of the cloned insert was confirmed.

The DNAs of pAT123 including the hygromycin resistance gene and pAT124 including the nourseothricin resistance gene (Example 6) were digested with AsiSI and the fragments spanning the genes purified using a gel extraction kit. These were ligated into pAT251 which had been digested with AsiSI and the ligation mixes introduced into E. coli DH5a competent cells. DNA was prepared from five colonies for each ligation and screened by digestion with restriction enzymes. The correct constructs were identified by agarose gel electrophoresis, confirming that the intended insertions had occurred between the 5′ upstream and 3′ downstream sequences. The resultant constructs having the Hyg or Nat1 antibiotic resistance gene sequences flanked by the 5′ upstream and 3′ downstream sequences from LRO1 were designated pAT261 and pAT262, respectively (Table 14 of Example 6).

Introduction of ARE1 Deletion Constructs into Y. lipolytica

To introduce the genetic constructs pAT261 and pAT262 into Y. lipolytica, the transformation protocol described in Example 6 was followed. Transformed cells were selected on YPD plates containing the appropriate antibiotic. Antibiotic resistant colonies were screened by PCR for the ARE1 gene insertion to identify genetically modified Δare1 cells from the transformation. DNA from W29 was used in parallel as a positive control for the PCRs. Primer pair at241 and at244 located in the 5′ upstream region and the 3′ downstream region, respectively, were used in PCR reactions to confirm the deletion mutation had been introduced into the genomic DNA of antibiotic resistant colonies. Additional PCR reactions with primer pairs at242 and at243, internal in the protein coding region, and at242 and at244 confirmed the presence of the deletion/insertion mutations. The absence of the ARE1 protein coding region was confirmed in 3 of 6 colonies resistant to hygromycin and 3 of 4 colonies resistant to nourseothricin. One of each of the transformed cell lines were retained as Y. lipolytica are1 deletion mutants and designated Y. lipolytica strain are1KO1(Hyg) and are1KO1(Nat1).

The are1KO1 strains are compared to the corresponding wild-type strain by growth in a high glucose/low nitrogen medium (DM-Glyc-LowN) that induces TAG synthesis, to determine the reduction in TAG synthesis ability. The level of TAG in the are1KO1 mutant was reduced by about 20%, so less than the reduction for dga1, dga2 and lro1. It was concluded that the Are1 protein was the least active in synthesizing TAG in Y. lipolytica of the four enzymes studied in this Example.

### Example 9. Modification of Microbes to Reduce Triacylglycerol Synthesis—Multi Gene Mutants

As described in Example 8, single gene mutants were produced in Y. lipolytica that had deletions in any one of four genes for TAG biosynthesis, namely DGA1, DGA2, LRO1 and ARE1. The inventors now aimed to produce mutants having multiple gene deletions, to further decrease endogenous TAG synthesis and to add in one or more genes to over-express a heterologous DGAT and/or other enzymes to modify the fatty acid composition such as fatty acyl thioesterases or acyl-CoA synthetases. This involved the removal first of all of an antibiotic resistance marker gene, for example the nourseothricin resistance gene, to allow re-use of the marker gene in a subsequent transformation.

### Cre-Lox Excision of Selectable Marker Genes

Where the selectable marker gene other than a hygromycin resistance gene was flanked by lox sites, the plasmid pUB4-CRE is used. This vector encodes a Cre recombinase protein which can excise the DNA between two lox sites. pUB4-CRE, which is a replicative vector from strain JME547, was obtained from INRAE, France (Fickers et al., 2003). The S. cerevisiae or Y. lipolytica strains to be modified are transformed with the plasmid pUB4-CRE as described below, selecting for hygromycin resistance. Colonies are plated on media with and without nourseothricin to screen for loss of the selectable marker gene. Colonies which are sensitive to the antibiotic are selected and the loss of the nourseothricin gene confirmed by PCR with flanking and internal primer combinations, and sequencing of the deletion region. A selected colony is grown in YPD medium in the absence of hygromycin i.e. without selection pressure and plated to identify a colony that has lost pUB4-CRE (Fickers et al., 2003). Such a colony is selected as the strain from which the nourseothricin selectable marker gene has been excised.

An analogous procedure is followed for excision of a hygromycin resistance selectable marker gene using a derivative of pUB4-CRE having a selectable marker gene other than the hygromycin resistance gene.

The transformation procedure to introduce pUB4-CRE is as follows using a Frozen-EZ Yeast Transformation II kit (Zymo Research, USA). The 50 μl of Ura-K021 competent cells, prepared as per instructions from Zymo Research are transformed with 0.5-1 μg DNA in a 5 to 10 μl volume. Then 500 μl EZ 3 solution is added mixed thoroughly with the cell suspension. The mix is incubated at 30° C. for between 45 min and 2 h, with occasional gentle mixing. 50-150 μl of the transformation mixture is spread on a YPD plate having hygromycin and lacking nourseothricin. The plates are incubated at 30° C. for 2-4 days to allow for growth of transformants.

### Generation of Mutants Having Multiple Inactivating Mutations

Once the hygromycin or nourseothricin resistance marker gene is excised from the mutated gene, for example the DGA1 gene, and the pUB4-CRE excision plasmid has been lost from the cells, the hygromycin or nourseothricin selectable marker gene can be used again in a second round of mutagenesis to inactivate a second gene. The process for marker excision can be repeated and a third round of mutagenesis carried out on a third gene, followed by fourth cycle of mutagenesis. With this strategy, double, triple and finally the quadruple mutants are generated for all combinations of the four TAG synthesis genes.

A similar strategy is followed with S. cerevisiae strain D5A to inactivate multiple genes selected from DGA1, LRO1, ARE1 and ARE2.

### Example 10. Modification of Microbes to Reduce Fatty Acid Catabolism

Y. lipolytica is considered to be an oleaginous yeast since it can produce more than 20% lipid by dry cell weight. The degradation and remobilization of lipids is driven by the β-oxidation pathway, which occurs in the peroxisome of microbes such as Y. lipolytica. Through this pathway, acyl-CoAs are catabolised via the activity of an acyl-CoA oxidase and the acyl chains are eventually broken down into acetyl-CoA molecules which are released from the peroxisome. Peroxisomal fatty acid β-oxidation is initiated by the activity of acyl-CoA oxidases, encoded by a single POX1 gene in S. cerevisiae and by six different POX genes, POX1 to POX6, in Y. lipolytica. The inventors considered that, by limiting the degradation of acyl-CoAs, the acyl chains could be utilised for the production and accumulation of lipids with increased saturated fatty acid content.

Experiments were therefore designed to reduce lipid catabolism through the inactivation of one or more genes encoding the most active acyl-CoA oxidase genes, including POX1-3 and 5, and MFE1, and also to interfere with the biogenesis of peroxisomes through the inactivation of the PEX10 gene in Y. lipolytica.

Genetic Constructs for Introducing a POX1 Gene Deletion into Y. lipolytica

To delete the protein coding sequence of the POX1 gene and other genes involved in β-oxidation of fatty acids from the Y. lipolytica genome, thereby providing null mutations, the general strategy is followed as described in Example 8 (FIG. 3). As before, the genetic cassette for introducing the gene deletions had the protein coding region of the gene of interest replaced with a selectable marker gene, flanked by 5′ upstream and 3′ downstream sequences of 1,000 basepairs which provided for integration of the genetic cassette by recombination into the endogenous gene. The primers used in the amplifications of the selectable marker genes had AsiSI restriction enzyme sites rather than SacII sites.

The nucleotide sequence of the POX1 gene of Y. lipolytica and its upstream and downstream sequences were extracted from the KEGG Yarrowia database (www.genome.jp/kegg-bin/show_organism?org=yli) using the published YARL1 gene identifier, as gene YALI0E32835 g, nucleotides 3897102 to 3899135 of chromosome E, Accession No. CR382131.1. The nucleotide sequence of the POX1 gene is provided herein as SEQ ID NO:60 including 1,000 nucleotides upstream of the protein coding sequence followed by the protein coding sequence and 1,000 nucleotides downstream of the protein coding sequence.

The amino acid sequence of the encoded POX1 polypeptide is provided as SEQ ID NO:61. Y. lipolytica POX1 is a protein of 677 amino acid residues.

The 5′ upstream and 3′ downstream regions adjacent to the POX1 protein coding region were amplified from genomic DNA from Y. lipolytica strain W29 (FIG. 3). Each amplification reaction used Taq DNA Polymerase with ThermoPol Buffer and a pair of oligonucleotide primers. By this means, the 5′ upstream fragment was adapted by adding restriction enzyme sites for AscI at its 5′ end and AsiSI at its 3′ end. Similarly, the 3′ downstream fragment was adapted by adding restriction enzyme sites for AsiSI at its 5′ end and NotI at its 3′ end. The amplified DNA fragments were digested with AsiSI and ligated with T4 DNA Ligase using standard protocols and inserted into vector into the vector pCR Zero Blunt TOPO. The nucleotide sequence of the cloned insert is confirmed.

The DNAs of pAT123 including the hygromycin resistance gene and pAT124 including the nourseothricin resistance gene (Example 6) were digested with AsiSI and the fragments spanning the genes purified using a gel extraction kit (Qiagen, USA). The DNA fragments are separately ligated with the amplified 5′ and 3′ regions which are digested with AsiSI and treated with calf intestinal alkaline phosphatase. The ligation mixes are transformed into E. coli DH5a competent cells. DNA is prepared from at least five colonies for each ligation and DNA samples from the colonies are screened by digestion with restriction enzymes and agarose gel electrophoresis to identify and confirm that the correct insertions had occurred between the 5′ upstream and 3′ downstream sequences. The resultant constructs having the Hyg or Nat1 antibiotic resistance gene sequences flanked by the 5′ upstream and 3′ downstream sequences from POX1 are selected and retained.

Introduction of POX1 Deletion Constructs into Y. lipolytica

To introduce the genetic construct containing the hygromycin resistance gene replacing the POX1 protein coding region into Y. lipolytica and identify genetically modified Δpox1 cells from the transformation, the transformation protocol described in Example 6 is followed. Transformed cells are selected on YPD plates containing 250 μg/mL hygromycin. Antibiotic resistant colonies are screened by PCR for the POX1 gene insertion. One of the transformed cell lines is selected and retained as a Y. lipolytica pox2 deletion mutant and designated strain pox1KO1.

The strain pox1KO1 is compared to its corresponding wild-type strain by growth in YPD, a rich medium, and a high glucose/low nitrogen medium that induces TAG synthesis, to determine the increase in TAG accumulation. The amount of lipid and the fatty acid composition is also assessed A genetic construct encoding a DGAT is introduced to increase TAG accumulation having an increased SFA content.

The same approach is used to delete the POX1 gene of S. cerevisiae (SEQ ID NO:76), encoding SEQ ID NO:77.

Genetic Constructs for Introducing a POX2 Gene Deletion into Y. lipolytica

To delete the protein coding sequence of the POX2 gene from the Y. lipolytica genome, the same strategy is used as for the POX1 deletion. The nucleotide sequence of the POX2 gene of Y. lipolytica and its upstream and downstream sequences were extracted from the KEGG Yarrowia database (www.genome.jp/kegg-bin/show_organism?org=yli), using the published YARL1 gene identifier, as gene YAL10F10857 g, nucleotides 1449289 to 1451391 of chromosome F, Accession No. CR382132.1. The nucleotide sequence of the POX2 gene is provided as SEQ ID NO:62 including 1,000 nucleotides upstream and downstream of the protein coding sequence. The amino acid sequence of the encoded POX2 polypeptide is provided as SEQ ID NO:63. Y. lipolytica POX2 is a protein of 700 amino acid residues.

Genetic Constructs for Introducing Other Targeted Gene Deletions into Y. lipolytica

An analogous process is followed to delete the coding region of the POX3 gene of Y. lipolytica (SEQ ID NO:64), encoding the POX3 protein (SEQ ID NO:65) that is also an acyl-CoA oxidase involved in catabolism of fatty acids.

To delete the protein coding sequence of MFE1 (SEQ ID NO:66) and PEX1-O (SEQ ID NO:68) which are both involved in peroxisome function and so mediate catabolism of fatty acids, the same approach is used. To delete the protein coding sequence of SNF1 (SEQ ID NO:70), SPO14 (SEQ ID NO:72) and OPI1 (SEQ ID NO:74) which are regulatory genes that regulate fatty acid synthesis, and other genes of interest from the Y. lipolytica genome, the same strategy is used as for the deletion of POX1, described above. The nucleotide sequences, including 1,000 nucleotides upstream and downstream of the protein coding sequence, and the encoded polypeptide sequences of the target genes have been provided (SEQ ID NOs: 67, 69, 71, 73, 75 and 77 herein), which were extracted from the KEGG Yarrowia database (www.genome.jp/kegg-bin/show_organism?org=yli), using the published YARL1 gene identifier.

### Example 11. Fatty Acid Composition in Single-Gene TAG Mutants

Each of the Y. lipolytica single gene mutants generated as described in Example 8 were grown in the DM-Glyc-LowN medium having an 8% (w/v) glycerol content and low nitrogen content to induce TAG production. Cultures were grown for 4 days at 28° C. The cells were harvested by centrifugation, washed twice with milliQ water, transferred to pre-weighed Eppendorf tubes, frozen in −80° C. for 20 min and freeze dried overnight. Lipids were extracted and fractionated to separate TAG from polar lipid and other lipid classes. The fatty acid composition of the TAG and polar lipid fractions were determined by GC of FAME as described in Example 1.

The data are shown in Table 17 for the average of duplicate cultures for each strain. Several significant features were noted. In this experiment, the wild-type strain W29 accumulated a TFA content of 6.3%. The single gene lro1 mutant accumulated slightly less total lipid. The single gene dga1 and dga2 mutants accumulated less TAG and therefore also less total lipid. This was not the case for the polar lipid content and composition which were much the same for each of the single gene mutants compared to the wild-type strain except for a slight increase in the SFA content in the polar lipids of the dga1 mutant. A much more substantial difference was noted, however, in the fatty acid content and composition of the TAG fraction. The TAG content was noticeably decreased in the dga1 and dga2 mutants, and also a decrease in the lro1 mutant but less so, which in each strain accounted for the reduction in total lipid. Most significantly, the percentages for the longer chain saturated fatty acids decreased markedly in the dga1 mutant, i.e. for C18:0, C20:0, C22:0 and most of all for C24:0. The C18:0 content in TAG decreased from 23.1% in the wild-type to 10.7% in the dga1 mutant, and C24:0 decreased from 7.5% to 1.3%, a relative decrease of 83%. In contrast, the C16:0 percentage decreased in the dga2 mutant. The same pattern was seen in the ratio of C18:0 and longer SFA to C16:0 and shorter SFA (L/S-SFA ratio), which decreased from 1.51 in the wild-type to 0.63 in the dga1 mutant, but increased to 1.97 in the dga2 mutant.

The inventors concluded that the different TAG-producing enzymes in Y. lipolytica differed in their fatty acid substrate specificity. That is, the DGA1 protein was active in inserting most of the longer SFA into TAG, in particular most of the C24:0 that accumulated in TAG, and so that enzyme had significantly greater specificity for longer SFA, whereas the DGA2 protein had greater specificity for C16:0. This also implied that most of the C24:0 esterified in TAG molecules in Y. lipolytica was esterified at the sn-3 position of the TAG molecules. The identity of the C24:0 in the peak on the GC trace was confirmed by GC-MS.

TABLE 17
Fatty acid content and composition of single-gene mutants of wild-type Y. lipolytica
strain W29 grown in defined medium with glycerol as sole C source, also showing
the total saturated fatty acid (SFA) content and the L/S-SFA ratio.

| | | | | | | | | | | |
|---|---|---|---|---|---|---|---|---|---|---|
| | | | | | | C16:1 | C16:1 | | | |
| Strain | C10:0 | C12:0 | C14:0 | C15:0 | C16:0 | Δ9 | Δ7 | C17:1 | C18:0 | C18:1 |

Polar lipid

| | | | | | | | | | | |
|---|---|---|---|---|---|---|---|---|---|---|
| are1 | 0.0 | 0.0 | 0.3 | 0.7 | 13.7 | 17.8 | 0.9 | 1.4 | 2.2 | 27.0 |
| dga1 | 0.0 | 0.0 | 0.4 | 0.7 | 16.7 | 15.8 | 1.3 | 1.4 | 2.6 | 28.7 |
| dga2 | 0.0 | 0.0 | 0.3 | 0.8 | 13.0 | 18.8 | 1.2 | 1.8 | 2.0 | 27.8 |
| Iro1 | 0.0 | 0.0 | 0.3 | 0.7 | 12.4 | 15.9 | 1.2 | 1.7 | 1.9 | 32.6 |
| W29 | 0.0 | 0.0 | 0.3 | 0.6 | 12.0 | 18.4 | 0.6 | 1.7 | 1.8 | 34.4 |

TAG

| | | | | | | | | | | |
|---|---|---|---|---|---|---|---|---|---|---|
| are1 | 0.0 | 0.0 | 0.4 | 0.6 | 19.3 | 10.6 | 0.7 | 0.6 | 18.9 | 17.7 |
| dga1 | 0.0 | 0.1 | 0.6 | 0.7 | 19.8 | 11.2 | 1.3 | 0.8 | 10.7 | 18.6 |
| dga2 | 0.0 | 0.0 | 0.3 | 0.6 | 13.5 | 11.3 | 1.1 | 0.9 | 17.1 | 19.2 |
| Iro1 | 0.0 | 0.1 | 0.4 | 0.6 | 18.9 | 9.0 | 1.2 | 0.7 | 20.2 | 19.0 |
| W29 | 0.0 | 0.0 | 0.4 | 0.5 | 20.9 | 9.1 | 0.4 | 0.7 | 23.1 | 19.4 |

TFA

| | | | | | | | | | | |
|---|---|---|---|---|---|---|---|---|---|---|
| Iro1 | 0.0 | 0.0 | 0.3 | 0.7 | 17.0 | 11.3 | 1.1 | 1.1 | 9.3 | 25.3 |
| W29 | 0.0 | 0.0 | 0.4 | 0.6 | 18.8 | 11.0 | 0.6 | 0.9 | 12.6 | 23.5 |

| | | | | | | | | | | |
|---|---|---|---|---|---|---|---|---|---|---|
| | | | | | | | | | | L/S- |
| | C18:1 | C18:2 | | C20:1 | | C22:1 | | Oil | Total | SFA |
| Strain | Δ11 | Δ9, 12 | C20:0 | Δ11 | C22:0 | Δ13 | C24:0 | % wt | SFA | Ratio |

Polar lipid

| | | | | | | | | | | |
|---|---|---|---|---|---|---|---|---|---|---|
| are1 | 0.8 | 32.5 | 1.0 | 0.2 | 0.1 | 0.1 | 0.9 | 1.9 | 19.0 | 2.8 |
| dga1 | 0.0 | 29.6 | 0.8 | 0.1 | 0.0 | 0.0 | 0.7 | 1.8 | 21.9 | 2.9 |
| dga2 | 0.9 | 31.0 | 0.8 | 0.1 | 0.0 | 0.0 | 0.7 | 2.1 | 17.7 | 3.2 |
| Iro1 | 0.0 | 30.6 | 0.9 | 0.1 | 0.1 | 0.0 | 1.0 | 1.9 | 17.3 | 3.1 |
| W29 | 0.0 | 29.3 | 0.1 | 0.1 | 0.0 | 0.0 | 0.3 | 1.7 | 15.1 | 2.9 |

TAG

| | | | | | | | | | | |
|---|---|---|---|---|---|---|---|---|---|---|
| are1 | 0.8 | 20.9 | 1.2 | 0.1 | 1.2 | 0.0 | 6.5 | 2.4 | 48.2 | 1.36 |
| dga1 | 0.0 | 33.0 | 0.7 | 0.1 | 0.5 | 0.0 | 1.3 | 1.4 | 34.5 | 0.63 |
| dga2 | 0.0 | 24.2 | 1.1 | 0.1 | 1.3 | 0.0 | 8.9 | 1.5 | 42.8 | 1.97 |
| Iro1 | 0.9 | 18.3 | 1.2 | 0.1 | 1.3 | 0.0 | 7.7 | 1.9 | 50.4 | 1.53 |
| W29 | 0.3 | 14.8 | 1.3 | 0.1 | 1.2 | 0.0 | 7.5 | 2.8 | 55.0 | 1.51 |

TFA

| | | | | | | | | | | |
|---|---|---|---|---|---|---|---|---|---|---|
| Iro1 | 0.7 | 24.9 | 0.9 | 0.1 | 0.6 | 0.0 | 5.8 | 6.0 | 34.6 | 0.92 |
| W29 | 0.0 | 24.6 | 1.0 | 0.2 | 0.8 | 0.0 | 4.3 | 6.3 | 38.7 | 0.94 |

### Example 12. Over-Expression of Y. lipolytica DGA1

In view of the data described in Example 11 and based on the conclusions drawn from the data, the inventors designed a genetic construct to over-express the Y. lipolytica DGA1 enzyme in yeast cells. For Y. lipolytica, the construct was specifically designed to integrate the coding region for the DGA1 protein under the control of a strong promoter such as a TEF promoter (SEQ ID NO:7), an FBAIn promoter (SEQ ID NO:78 or SEQ ID NO:79) or a PDK promoter (SEQ ID NO:80). The heterologous DGA1 gene had flanking sequences from the DGA2 gene (Example 8) for integration, resulting in inactivation of the DGA2 gene. This construct was designed to simultaneously provide two genetic modifications, namely to both over-express the DGA1 polypeptide and to inactivate the DGA2 gene in Y. lipolytica, to increase SFA content further than either genetic modification alone. Transformed strains having the construct integrated into the DGA2 gene are grown in media to induce TAG synthesis. Lipid extracted from these cultures have much increased SFA content relative to the untransformed parental strain, including substantially increased C20:0, C22:0 and C24:0 in the total fatty acid content of the extracted lipid.

### Example 13. Modification of Microbes to Increase Saturated Fatty Acid Content

Many yeasts produce polyunsaturated fatty acids (PUFA) including linoleic acid (LA, C18:2Δ9,12) and, in some species, α-linolenic acid (ALA, C18:3Δ9,12,15) which are incorporated into their lipids, including in TAG. See for instance Example 3 above. The present inventors wished to increase the level of saturated fatty acids including stearic acid (C18:0) and reduce polyunsaturated fatty acids (PUFA) including the ω6 fatty acid LA to increase the melting point of the extracted lipid. When cultured in a rich medium having glucose as carbon source and lacking fatty acid supplementation, the wild-type Y. lipolytica strain W29 produced relatively low amounts of stearic acid at about 4-7% and about 6-15% of LA, each as a percentage of the total fatty acid content of the cells (Table 7, Example 3). When afad2 mutant strain cultured in a medium having low nitrogen levels and glycerol as carbon source to induce more TAG synthesis, the stearic acid level increased to 18% (Table 16, Example 6) or upward of 20% in other experiments. Strain W29 lacked an active Δ15 desaturase since ALA was absent from the TAG and phospholipid extracted from the cells. As described in Example 6, inactivation of the FAD2 gene in Y. lipolytica by mutation abolished the production of PUFA, specifically LA and other ω6 fatty acids.

An experiment was carried out culturing wild-type strain W29, the fad2 mutant and the fad2-ura3 double mutant in a defined medium with a high carbon:nitrogen ratio (DM-Glyc-LowN, Example 1) to increase production and accumulation of TAG. The starter cultures were grown in SD-Ura medium and used to inoculate the cultures in DM-Glyc-LowN. These cultures were grown for 96 h. The pH was measured daily and adjusted to pH 6.0 with NaOH at 24 h and 48 h, after which it stabilised and did not need further adjustment. The cells were harvested at 96 h and washed. The total fatty acid content in cell samples was measured by direct methylation of the cells as described in Example 1, and lipid was extracted from the remainder of the cells and fractionated by TLC. The TAG fraction was analyzed by GC as described in Example 1. Under the culture conditions used, the TAG from strain W29 showed an increase in stearic acid content to about 31-33% and a low content of LA at about 4-5% (Table 18), relative to previous experiments. The total saturated fatty acid content was 52-55% of the total fatty acid content, so higher than had been seen before. Palmitic acid was present at 16-20% by weight of the TFA content. All of the samples also contained C20:0, C22:0 and C24:0. The TAG from the fad2 and fad2-ura3 mutants lacked LA and other PUFA and had increased amounts of oleic acid, the primary substrate for Δ12 desaturase encoded by the FAD2 gene. These mutants also had slightly less stearic acid at 27-30%, but about the same palmitate level at 15-19% of the TFA content. The TFA content under these growth conditions reached about 6% by dry cell weight. The inventors concluded that the percentage of stearic acid and other longer chain SFA in the total fatty acid content of the extracted lipid could be significantly affected by the culture medium and growth conditions used. The predicted melting point of the TAG fraction of the ura3-pAT207 and ura3-pAT208 mutants is about 40° C. The predicted melting point of the TAG fraction of the fad2-ura3 double mutant (ura3fad2 pAT207 and ura3fad2 pAT208) is about 44° C.

TABLE 18
Fatty acid composition of TAG extracted from Y. lipolytica
cells after culturing in a low nitrogen medium for 96 h.
Results are shown for duplicate or triplicate cultures.

| | | | | C16:1 | C16:1 | | |
|---|---|---|---|---|---|---|---|
| Strain | C14:0 | C15:0 | C16:0 | Δ9 | Δ7 | C17:1 | C18:0 |
| W29-1 | 0.2 | 0.2 | 19.0 | 2.6 | 0.7 | 0.3 | 32.4 |
| W29-2 | 0.2 | 0.2 | 16.9 | 2.8 | 0.5 | 0.4 | 31.7 |
| W29-3 | 0.2 | 0.2 | 18.3 | 2.4 | 0.6 | 0.3 | 33.2 |
| fad2KO1 | 0.2 | 0.3 | 15.5 | 3.5 | 1.6 | 0.5 | 27.8 |
| fad2KO5 | 0.2 | 0.2 | 15.1 | 3.3 | 1.4 | 0.4 | 29.1 |
| fad2KO6 | 0.2 | 0.2 | 16.4 | 3.4 | 1.4 | 0.4 | 28.3 |
| fad2-ura3 (21) | 0.2 | 0.2 | 18.5 | 3.0 | 1.3 | 0.4 | 29.3 |
| fad2-ura3 (23) | 0.2 | 0.2 | 17.9 | 2.6 | 0.9 | 0.3 | 30.4 |

| | | | | | | | lipid |
|---|---|---|---|---|---|---|---|
| | C18:1 | C18:1 | | | | | content |
| | Δ9 | Δ11 | C18:2 | C20:0 | C22:0 | C24:0 | (% dcw) |
| W29-1 | 37.5 | 0.1 | 3.9 | 1.1 | 0.6 | 1.3 | 6.2 |
| W29-2 | 39.6 | 0.1 | 4.6 | 1.0 | 0.6 | 1.5 | 6.1 |
| W29-3 | 37.1 | 0.0 | 4.2 | 1.2 | 0.7 | 1.7 | 4.8 |
| fad2KO1 | 47.6 | 0.2 | 0.0 | 0.9 | 0.6 | 1.4 | 2.8 |
| fad2KO5 | 47.1 | 0.2 | 0.0 | 1.0 | 0.6 | 1.4 | 4.3 |
| fad2KO6 | 46.8 | 0.2 | 0.0 | 0.9 | 0.5 | 1.3 | 4.8 |
| fad2-ura3 (21) | 44.0 | 0.2 | 0.0 | 0.9 | 0.5 | 1.3 | 5.6 |
| fad2-ura3 (23) | 44.4 | 0.2 | 0.0 | 1.0 | 0.6 | 1.5 | 4.4 |

The following were not detected in the samples: C8:0, C10:0, C18:3ω3, C18:3ω6, C22:2ω6.

### Genetic Modifications to Increase Saturated Fatty Acid Content

The inventors wished to further increase the level of total SFA in microbial lipids, in particular in yeasts such as Y. lipolytica. As described in Examples 11 and 12 and this Example, they conceived of ways to increase the expression in the microbial cells of a DGAT which had activity to transfer a SFA into TAG, in particular if the DGAT had at least the same activity, preferably greater activity, on the longer SFA-CoA substrates such as stearoyl-CoA, C20:0-CoA, C22:0-CoA and especially C24:0-CoA relative to C16:0-CoA. That is, a DGAT that had greater specificity for SFA having at least C18 compared to other DGATs. As described in Examples 11 and 12, one such DGAT was the Y. lipolytica DGA1 protein, which was more related in amino acid sequence to animal and plant DGAT2 than DGAT1 proteins, but which nevertheless had greater activity on stearate and longer SFA than the DGA2 protein. As described in this Example, other DGAT enzymes which were known to have greater specificity for monounsaturated fatty acids (MUFA) and were therefore not expected to increase the percentage of SFA, were surprisingly able to substantially increase the percentage of stearic acid and total SFA in the extracted lipid of microbes such as Y. lipolytica, as well as increasing the total lipid content of the cells when grown under conditions favouring TAG production. In some of these experiments, the genetic construct that was introduced into the microbial cells had a second gene encoding a fatty acyl thioesterase which had activity on stearoyl-ACP, such as for example the mangosteen thioesterase.

The inventors identified a DGAT1 enzyme from macadamia, Macadamia tetraphylla, designated as MtDGAT1, which was reported by Arroyo-Caro et al. (2015) to have substrate specificity for MUFA over PUFA. Macadamia can accumulate an unusually high percentage of MUFA in its seedoil, having about 60% oleic and 20% palmitoleic acids in the total fatty acid content. The present inventors selected MtDGAT1 with the intention of testing it for increased production of MUFA in Y. lipolytica, not thinking it might increase SFA and decrease MUFA content. The amino acid sequence of MtDGAT1, NCBI Accession No. KT736302, is provided herein as SEQ ID NO:81. It has 535 amino acids and has domains for binding acyl-CoA and DAG as substrates, nine or ten predicted transmembrane domains and an ER retention motif close to the C-terminus (Arroyo-Caro et al., 2015). Over-expression of MtDGAT1 in S. cerevisiae cells that were defective in TAG synthesis produced lipids with increased levels of MUFA, in particular oleic acid, but levels of SFA (C16:0 and C18:0) were similar to those produced by over-expression of A. thaliana DGAT1 and Echium pitardii DGAT1 in the same strain of S. cerevisiae (Arroyo-Caro et al., 2015). The authors did not report any enhancement of SFA in the yeast cells and did not expect any. Furthermore, other studies with plant DGAT1 enzymes showed substrate specificity for monounsaturated oleoyl-CoA, with no increase in C16:0 and C18:0 (Zhou et al., 2013).

The inventors also selected two related acyl-acyl-carrier-protein (ACP) thioesterases from mangosteen, Garcinia mangostana, designated GarmFATA1 and GarmFATA2 (Hawkins and Kridl, 1998). Both proteins have an N-terminal transit peptide sequence (TPS) which function to direct the proteins into plastids. The former polypeptide including its TPS has 352 amino acids whereas the latter including its TPS has 355 amino acids. The amino acid sequences of GarmFATA1 and GarmFATA2 are 73% identical along the full length of GarmFATA1. Acyl-ACP thioesterases cleave acyl groups from ACP and thereby terminate the acyl chain elongation activity in fatty acid synthesis by fatty acid synthase (FAS). Fatty acyl-ACP thioesterases are classified into two distinct but related groups on the basis of sequence homology and activity, namely FATB thioesterases which have specificity mainly for C16 and shorter SFA, and FATA thioesterases which are more active on C18-ACP than C16-ACP substrates, particularly MUFA-ACP substrates. GarmFATA1 had its greatest activity on C18:1-ACP, with about 8-fold less activity on C18:0-ACP and less again on C16:0-ACP. In contrast, GarmFATA2 had about 50-fold less activity on C18:0-ACP relative to C18:1-ACP. Both enzymes therefore had their main activity for the MUFA, C18:1-ACP. Heterologous expression of GarmFATA1 in Brassica seeds led to the accumulation of 22% stearic acid in the seedoil in one instance (Hawkins and Kridl, 1998). The amino acid sequences of GarmFATA1 and GarmFATA2 are provided herein as SEQ ID NO:83 and SEQ ID NO:85.

For another project, the inventors had selected an acyl-CoA synthetase (ACS) from Pseudomonas chlororaphis which had activity on short chain fatty acids (SCFA) (Hashimoto et al., 2005). Acyl-CoA synthetases catalyse the covalent bonding of fatty acids to a cofactor A (CoA) molecule to produce the acyl-CoA thiol esters in an ATP dependent reaction. The enzyme was reported to be inactive on longer chain fatty acids (Hashimoto et al., 2005) and was included in the constructs described below even though they were not expected to affect longer chain SFA. The amino acid sequence of PcACS is provided herein as SEQ ID NO:87.

The inventors also selected a M alpina LPAAT (MaLPAAT) which was thought to have substrate specificity for PUFA, included in two constructs, again for a different purpose. The amino acid sequence of MaLPAAT is provided herein as SEQ ID NO:90.

Construction of pAT207 to pAT212

The GoldenGate strategy was used to generate multi-gene constructs as described in Example 1. As a first step, intermediate genetic constructs were made which included coding regions for the desired polypeptides, each construct having a single gene insertion in a cloning vector. Each protein coding region was codon optimised for expression in Y. lipolytica and synthesized with flanking BsaI restriction sites (GGTCTC) to allow the GoldenGate assembly. A first genetic construct, pAT117, was synthesized which had the protein coding region of the macadamia DGAT1. The codon optimised nucleotide sequence of the protein coding region of 1588 nucleotides (SEQ ID NO:82) was 75% identical to the native macadamia sequence in NCBI Accession No. KT736302.1, i.e. 25% of the nucleotides were changed by the codon optimization. Second and third genetic constructs, pAT066 and pAT067, were synthesized having protein coding regions encoded the GmFATA1 and GmFATA2 polypeptides, respectively. The codon optimised nucleotide sequences of the protein coding regions were 74% identical to the native mangosteen sequences. The nucleotide sequences of the protein coding regions in pAT066 and pAT067 are provided as SEQ ID NO:84 and SEQ ID NO:86, respectively. Fourth and fifth genetic constructs, pAT136 and pAT138, were synthesized having protein coding regions encoded the same PcACS polypeptide (SEQ ID NO:87) but differing in their nucleotide sequences. The codon optimised nucleotide sequences of the protein coding regions were 65% identical to the native Pseudomonas sequence. The nucleotide sequences of the protein coding region in pAT136 and pAT138 are provided as SEQ ID NO:88 and SEQ ID NO:89, respectively.

These intermediate vectors were used in GoldenGate assembly reactions together with a pTEF promoter sequence and a Lip2 gene transcription terminator/polyadenylation region for each heterologous gene to produce a series of genetic constructs, each containing 4 genes including a Ura3 gene as the selectable marker gene. The Ura3 gene was present in the destination vector GGE114. DNA parts for pAT207, for example, were from GGE146, GGE151 and GGE294 (pTEF promoters), GGE014, GGE015, GGE080, GGE020 and GGE021 (terminators) and the backbone assembly vector (destination vector) was GGE114, in addition to the coding regions from pAT066, pAT136 and pAT117. The pTEF promoters used to drive each gene other than the URA3 gene comprised 4 concatenated upstream activation sequences (UAS), each of 109 nucleotides in length, which together acted as an enhancer element for increased transcription of the genes. The other constructs were assembled in analogous fashion. The genes in each construct are listed in Table 19. Each 4-gene cluster was flanked by NotI restriction enzyme sites to allow for excision of the DNA fragment with NotI. The 4-gene clusters were also flanked by a 5′ portion of the Y. lipolytica zeta sequence just inside the 5′ NotI site, and a 3′ portion of the Y. lipolytica zeta sequence just inside the 3′ NotI site to provide for more efficient integration into the Y. lipolytica genome. The nucleotide sequences for the six constructs are provided as SEQ ID NOs:92 to 97.

TABLE 19
Genetic constructs and component genes

| | | | | | SEQ |
|---|---|---|---|---|---|
| Genetic | Gene | Gene | Gene | Gene | ID |
| construct | position 1 | position 2 | position 3 | position 4 | NO |
| pAT207 | Ura3 | GmFATA1 | PcACS-X1 | MtDGAT1 | 92 |
| pAT208 | Ura3 | GmFATA1 | PcACS-X2 | MtDGAT1 | 93 |
| pAT209 | Ura3 | GmFATA2 | PcACS-X1 | MtDGAT1 | 94 |
| pAT210 | Ura3 | GmFATA2 | PcACS-X2 | MtDGAT1 | 95 |
| pAT211 | Ura3 | GmFATA1 | MaLPAAT | MtDGAT1 | 96 |
| pAT212 | Ura3 | GmFATA2 | MaLPAAT | MtDGAT1 | 97 |

Transformation of Y. lipolytica and Analysis of Lipids Produced by the Transformants

In an initial experiment, the NotI DNA fragments from pAT207-210 were used to transform the ura3KO27 mutant strain of Y. lipolytica produced as described in Example 7. Transformants were selected for uracil prototrophy on SD-Ura plates. Colonies were screened and positive, transformed colonies were identified. Five transformants from each transformation were cultured for 96 h in DM-Glyc-LowN medium, lipid extracted and fractionated on TLC plates. The fatty acid composition and content of the TAG and polar lipid fractions were analysed by GC of FAME. The data of selected transformants from pAT207 to pAT210 are presented in Tables 20 and 21, showing data for triplicate cultures of each transformant. Other transformants had similar fatty acid composition compared to the control strain or were intermediate in phenotype and were presumed to have lower expression levels, or no expression, of the introduced transgenes. This observation was expected for constructs that insert into the host genome via the zeta sequence, which can insert in any of many possible sites within the genome.

Several observations were significant. Firstly, each of the constructs pAT207, 208 and 209 provided transformants that were significantly increased in the stearic acid content as a percentage of the total fatty acid content in both the TAG content and the polar lipid content of the cells. The stearic acid content in transformant pAT209-5 reached 37.9% of the total fatty acid content in TAG and 35.5% in polar lipid, compared to about 20% in TAG and about 3% in polar lipid in the control strain ura3KO27. Moreover, the TAG content was more than doubled in some transformants relative to the untransformed control strain. Considering that these transformants were not grown under optimal conditions for TAG synthesis (see below), the degree of increase of TAG was not maximal. From these data, the inventors concluded that the amount of stearic acid being produced in the transformants was substantially increased as well as being increased in incorporation into both TAG and polar lipid. The amount of stearic acid being produced was considered to be increased both in absolute amount and relative to other fatty acids. This suggested that both the acyl-ACP thioesterase and DGAT1 enzymes produced from the genes in pAT207, 208 and 209 were both functioning to increase stearate production and incorporation into the cellular lipids. The increase in stearate incorporation into TAG pointed to activity of the MtDGAT1. The inventors considered that the PcACS in those constructs would not have had any role in increasing stearate levels, given its known lack of activity on fatty acids other than SCFA.

The inventors also observed that the VLC-SFA, C20:0 and C22:0, were increased by up to about 4-fold in the TAG from the transformants, in addition to the increase in stearate. C20:0 was also increased in the polar lipid from the transformants. This indicated that there was an increased production and incorporation into the cellular lipids of VLC-SFA in the transformants along with the increased stearate. A further observation was that the TAG content, but not the polar lipid content, was substantially increased in the transformants relative to the untransformed control strain. This indicated that the DGAT gene was active in the transformants, even on its own without any activity of the thioesterase enzyme which would not be expected to increase TAG levels, including incorporating an increased amount of stearate and longer SFA into TAG relative to the untransformed cells. Furthermore, there was a significant decrease in the percentage of monounsaturated fatty acids such as C18:1Δ9 and C16:1Δ9 in the TAG from the transformants. This was a surprising result for the constructs expressing the MtDGAT1 since it was reported to have its primary activity on these MUFA (Arroyo-Caro et al., 2015), indeed it was the opposite result to what the inventors had expected. A similar decrease in the MUFA content was observed in the polar lipids from the transformants.

Furthermore, the total SFA content in the TAG from the transformants was increased from below 50% to more than 60%, and the L/S-SFA ratio was increased from below 1.5 to more than 3.0. This, again, was an unexpected result in view of the reported primary activity of the MtDGAT1 enzyme on MUFA.

### Transformation of Fad2 Mutant and Analysis of Lipids Produced by the Transformants

The NotI DNA fragments from pAT207 to pAT212 were also used to transform the fad2KO1-uraKO27 double mutant strain of Y. lipolytica produced as described in Example 7. The transformation procedure was performed as described by Chen et al. (1997) and putative transformant colonies were selected on SD-ura plates incubated at 30° C. for 3 days. The procedure was carried out twice for pAT207, pAT208, pAT210, pAT211 and pAT212, and three times with the pAT209 plasmid. Genomic DNA was extracted from the putative transformants as described by Looke et al. (2011) and the presence of the genomic integrated pAT207-212 plasmids confirmed by PCR amplification using the oligonucleotide primer pair at001 (SEQ ID NO:98) and at002 (SEQ ID NO:99). In a first set of transformations, the number of confirmed transformed colonies were: pAT207, 4; pAT208, 10; pAT209, 8, pAT210, 9; pAT211, 10 and pAT212, 7. In a second set, the numbers of selected colonies were: pAT207, 8; pAT208, 3; pAT209, 33; pAT210, 10; pAT211, 5 and pAT212, 10. These were re-streaked onto SD-ura plates. A single colony from each confirmed transformant strain was selected and cultured in SD-ura medium overnight to produce a starter culture. The starter cultures were used to seed 10 ml DM-Glyc-LowN medium in 50 ml Falcon tubes to an OD600 of 0.2, covered with sterile AeraSeal™ film (Sigma, A9224) and cultured for 96 h at 30° C. with shaking at 200 rpm for aeration.

Lipid was extracted from the cell cultures and lipids extracted and analysed as described in Example 1. The fatty acid content and composition of the total fatty acid (TFA), the TAG and polar lipid fractions were analysed by GC of FAME. The data for selected transformants from pAT207 to pAT212 are presented in Tables 22 to 24.

It was immediately apparent that the total fatty acid content was greatly increased in many of the transformants relative to the untransformed control. Some of the transformants had total fatty acid contents of up to about 33% on a weight basis (dry cell weight) compared to the untransformed cells which yield between 10-13% TFA on a weight basis. This was in combination with reduced PUFA, in particular reduced LA which was either not detected or present at not more than 0.2%, for example at a trace amount of 0.1% by weight of the total fatty acid content. Other transformants were not increased in TFA content or were intermediate in this phenotype, indicating a range of transgene activities for different transformants. As for the previous experiment with the FAD2 wild-type strain, substantial increases in the stearic acid, C20:0 and C22:0 contents were observed relative to the untransformed control strain, both in the absolute amounts of the fatty acids, considering the increased lipid content, and in the amounts relative to other fatty acids. The stearate content reached almost 40% in some transformants. In some transformants, the amount of C20:0 exceeded the amount of C24:0 on a weight basis, being at least 1.0% of the TFA content and in some transformants exceeding 3.0% of both the TFA content and the fatty acid content of TAG on a weight basis. In another transformant, the extracted lipid, and the TAG in the lipid, had 12% C24:0, so the C24:0 amount was substantially greater than the C20:0 and C22:0 amounts. The total SFA content was increased to up to about 70% by weight of the TFA content. The L/S-SFA ratio was also significantly increased from about 1.2-1.4 in the control cells to above 3.0 in some instances, e.g colony pAT208-5. These data again pointed to the activity of both the DGAT and FATA enzymes in some of the transgenic strains. Palmitate contents were also increased in some transformants, contributing to increases in the total SFA. In each case, both the LA content and the total PUFA content was not more than 0.2%, so LA was either absent or was present at less than 0.2% on a weight basis, due to the fad2 mutation and the lack of any other Δ12 desaturase. The amount of oleic acid was reduced from about 55-60% in the untransformed cells to between 25% and 55% in the transformed cells, on a weight basis as a percentage of the TFA content. Interestingly, the C24:0 content in TFA and in TAG was not increased in the transformants relative to the untransformed cells, even though the C20:0 and C22:0 contents were substantially increased, indicating that the elongase that elongates C22:0 to C24:0 was not increased in its activity in these Y. lipolytica cells. C24:0 is used by Y. lipolytica cells mostly in the sphingolipid content for membrane function, even though some C24:0 was esterified in the form of TAG in these cells.

It was also observed that some of the transformants had increased TAG content but not substantially increased stearate content, while other transformants had increased stearate content but TAG content not increased as much. It was considered that this was likely due to one of the two exogenous enzymes DGAT and FATA being more active than the other, namely the DGAT being more responsible for increasing TAG content and the FATA thioesterase more responsible for the increased stearate relative to the other fatty acids. It was also observed that some of the transformants had decreased stearate content relative to the wild-type e.g. colonies pAT212-1 and 212-4 (Table 22). This may have resulted from a decrease in an endogenous thioesterase activity. RT-PCR assays for the mRNA levels in the different transformants for these transgenes and endogenous genes are carried out to confirm these conclusions.

The transformants from pAT211 and pAT212 appeared to provide a greater number of transformants that had substantially increased stearate content and SFA content than the other four constructs, as a percentage of the number of transformants obtained. This may have indicated some activity of the LPAAT-encoding gene on these genetic constructs. It was noted that these two constructs did not have a gene encoding the acyl-CoA synthetase (ACS) from Pseudomonas—that PcACS was not expected to have an effect on the fatty acids having at least C18.

The data from the analysis of polar lipid from the transformants (Table 22) supported the conclusions described above but also revealed some surprising observations. Firstly, the polar lipid content was significantly increased from between 2% and 3% in the untransformed cells to at least 8% in some transformants. This was observed more in the transformants that included the gene expressing an LPAAT than those that didn't, from which it was concluded that the LPAAT had a role in increasing polar lipid content. Secondly, not only the stearate content but also the palmitate content was greatly increased in some transformants, resulting in a SFA content in the polar lipid of more than 60% by weight. Surprisingly, the SFA content as a percentage of the total fatty acids in the polar lipid in some transformants exceeded the SFA content in the TAG content. This was most surprising in view of the much lower SFA content of about 11% in the polar lipid of the untransformed cells compared to the SFA content of about 40% in the TAG of the untransformed cells. The increase in palmitate in the polar lipid of the transformed cells was not expected in view of the slight decrease in TAG in the same cells. It was concluded that the increase in SFA in the polar lipid was likely due to the exogenous FATA thioesterase activity increasing the supply of SFA.

TABLE 20
Fatty acid composition of TAG from selected transformed Y. lipolytica ura3KO27 cells, from triplicate cultures.

| | | | | | | | | | | | | | L/S- | Lipid |
|---|---|---|---|---|---|---|---|---|---|---|---|---|---|---|
| | | | C16:1 | | C18:1 | C18:1 | C18:2 | | C20:1 | | | Total | SFA | content |
| Transformant | C14:0 | C16:0 | Δ9 | C18:0 | Δ9 | Δ11 | Δ9, 12 | C20:0 | Δ11 | C22:0 | C24:0 | SFA | ratio | (% dcw) |
| KO27-1 | 0.3 | 20.8 | 5.6 | 23.3 | 32.3 | 0.5 | 11.9 | 1.0 | 0.1 | 0.8 | 3.5 | 49.6 | 1.36 | 3.0 |
| KO27-2 | 0.4 | 17.9 | 7.6 | 17.5 | 32.4 | 0.8 | 17.4 | 0.9 | 0.1 | 0.8 | 4.0 | 41.6 | 1.27 | 1.9 |
| KO27-3 | 0.4 | 16.6 | 7.7 | 18.2 | 30.3 | 0.9 | 19.1 | 1.0 | 0.1 | 1.0 | 4.6 | 41.8 | 1.45 | 1.5 |
| pAT207-4 | 0.4 | 13.3 | 4.0 | 32.3 | 26.5 | 0.3 | 12.6 | 3.0 | 0.0 | 3.5 | 4.1 | 56.6 | 3.12 | 3.5 |
| pAT207-4 | 0.5 | 14.2 | 3.6 | 37.3 | 25.0 | 0.3 | 11.6 | 3.1 | 0.0 | 2.5 | 1.9 | 59.5 | 3.05 | 4.3 |
| pAT207-4 | 0.4 | 12.8 | 3.6 | 34.1 | 25.8 | 0.3 | 12.0 | 3.2 | 0.0 | 3.7 | 4.1 | 58.2 | 3.42 | 3.8 |
| pAT208-1 | 0.4 | 13.0 | 3.2 | 37.4 | 23.9 | 0.3 | 11.8 | 3.6 | 0.0 | 3.3 | 3.0 | 60.7 | 3.51 | 3.6 |
| pAT208-1 | 0.4 | 12.6 | 2.9 | 35.0 | 25.6 | 0.3 | 12.1 | 3.3 | 0.1 | 0.0 | 4.3 | 55.7 | 3.28 | 2.5 |
| pAT208-1 | 0.4 | 14.3 | 4.5 | 28.1 | 26.6 | 0.3 | 15.0 | 2.7 | 0.0 | 3.4 | 4.6 | 53.5 | 2.63 | 3.0 |
| pAT209-4 | 0.4 | 14.4 | 4.8 | 30.3 | 23.7 | 0.3 | 15.4 | 3.1 | 0.0 | 3.3 | 4.0 | 55.6 | 2.74 | 2.9 |
| pAT209-4 | 0.5 | 13.8 | 4.2 | 32.7 | 23.3 | 0.3 | 15.3 | 3.5 | 0.0 | 3.1 | 3.2 | 56.7 | 2.99 | 3.7 |
| pAT209-4 | 0.4 | 15.0 | 3.6 | 34.6 | 22.0 | 0.3 | 13.4 | 3.5 | 0.0 | 3.3 | 3.9 | 60.7 | 2.95 | 3.8 |
| pAT209-5 | 0.4 | 15.0 | 3.1 | 37.5 | 16.3 | 0.2 | 16.9 | 4.0 | 0.0 | 3.5 | 3.0 | 63.3 | 3.12 | 4.2 |
| pAT209-5 | 0.4 | 14.4 | 3.2 | 37.9 | 17.5 | 0.2 | 15.7 | 4.1 | 0.0 | 3.6 | 3.0 | 63.3 | 3.28 | 4.8 |
| pAT209-5 | 0.4 | 14.6 | 3.5 | 34.1 | 18.0 | 0.2 | 17.4 | 3.4 | 0.0 | 3.8 | 4.4 | 60.8 | 3.05 | 4.1 |
| pAT210-2 | 0.5 | 15.5 | 7.4 | 16.5 | 33.1 | 0.9 | 19.4 | 1.0 | 0.1 | 1.0 | 4.5 | 39.0 | 1.44 | 1.2 |
| pAT210-2 | 0.6 | 15.4 | 6.9 | 17.9 | 29.3 | 0.9 | 21.7 | 1.2 | 0.1 | 1.2 | 4.6 | 41.1 | 1.55 | 1.3 |
| pAT210-2 | 0.4 | 20.4 | 6.4 | 21.9 | 29.5 | 0.6 | 14.3 | 1.3 | 0.0 | 1.1 | 4.2 | 49.3 | 1.37 | 1.5 |

TABLE 21
Fatty acid composition of polar lipids from selected Y. lipolytica cells transformed with one of pAT207 to pAT210, from triplicate cultures.

| | | | | | | | | | | | | | L/S- | Lipid |
|---|---|---|---|---|---|---|---|---|---|---|---|---|---|---|
| | | | C16:1 | | C18:1 | C18:1 | C18:2 | | C20:1 | | | Total | SFA | content |
| Transformant | C14:0 | C16:0 | Δ9 | C18:0 | Δ9 | Δ11 | Δ9, 12 | C20:0 | Δ11 | C22:0 | C24:0 | SFA | ratio | (% dcw) |
| KO27-1 | 0.2 | 14.7 | 9.8 | 3.2 | 46.3 | 0.6 | 24.2 | 0.0 | 0.0 | 0.5 | 0.5 | 19.1 | 0.28 | 0.9 |
| KO27-2 | 0.3 | 14.8 | 10.5 | 2.9 | 44.2 | 0.7 | 25.7 | 0.0 | 0.0 | 0.4 | 0.5 | 18.9 | 0.25 | 0.9 |
| KO27-3 | 0.4 | 14.9 | 11.2 | 0.2 | 42.6 | 0.7 | 29.3 | 0.0 | 0.0 | 0.4 | 0.4 | 16.2 | 0.06 | 1.0 |
| pAT207-4 | 0.5 | 16.0 | 7.9 | 8.2 | 40.4 | 0.4 | 25.3 | 0.4 | 0.0 | 0.2 | 0.6 | 25.9 | 0.57 | 0.9 |
| pAT207-4 | 0.5 | 20.2 | 5.8 | 16.7 | 34.4 | 0.4 | 20.5 | 0.7 | 0.0 | 0.3 | 0.6 | 38.9 | 0.88 | 0.9 |
| pAT207-4 | 0.5 | 15.9 | 7.6 | 7.5 | 41.2 | 0.4 | 26.1 | 0.3 | 0.0 | 0.0 | 0.4 | 24.7 | 0.50 | 0.9 |
| pAT208-1 | 0.6 | 18.6 | 6.3 | 13.5 | 35.8 | 0.4 | 23.3 | 0.6 | 0.0 | 0.3 | 0.6 | 34.2 | 0.78 | 0.7 |
| pAT208-1 | 0.6 | 17.0 | 5.7 | 12.0 | 38.1 | 0.4 | 24.7 | 0.6 | 0.0 | 0.3 | 0.6 | 31.1 | 0.77 | 0.7 |
| pAT208-1 | 0.6 | 15.4 | 8.5 | 5.3 | 37.4 | 0.4 | 31.2 | 0.3 | 0.0 | 0.2 | 0.7 | 22.5 | 0.41 | 0.7 |
| pAT209-4 | 0.5 | 16.5 | 9.0 | 6.8 | 34.5 | 0.4 | 31.2 | 0.4 | 0.0 | 0.2 | 0.5 | 25.0 | 0.47 | 0.8 |
| pAT209-4 | 0.6 | 17.8 | 7.2 | 10.5 | 31.4 | 0.4 | 30.3 | 0.7 | 0.0 | 0.4 | 0.7 | 30.6 | 0.66 | 0.9 |
| pAT209-4 | 0.4 | 18.4 | 7.5 | 11.5 | 34.0 | 0.4 | 25.9 | 0.6 | 0.0 | 0.3 | 0.8 | 32.1 | 0.70 | 1.0 |
| pAT209-5 | 0.6 | 23.9 | 3.4 | 33.3 | 14.7 | 0.0 | 21.5 | 1.4 | 0.0 | 0.6 | 0.6 | 60.4 | 1.47 | 1.1 |
| pAT209-5 | 0.6 | 24.1 | 3.2 | 35.5 | 14.8 | 0.0 | 18.4 | 1.5 | 0.0 | 0.5 | 1.4 | 63.6 | 1.58 | 1.0 |
| pAT209-5 | 0.6 | 20.2 | 6.4 | 15.1 | 22.8 | 0.2 | 33.1 | 0.7 | 0.0 | 0.3 | 0.6 | 37.4 | 0.81 | 1.0 |
| pAT210-2 | 0.4 | 14.7 | 10.8 | 0.1 | 42.0 | 0.7 | 30.2 | 0.1 | 0.0 | 0.4 | 0.4 | 16.2 | 0.08 | 1.1 |
| pAT210-2 | 0.5 | 14.7 | 10.3 | 2.9 | 37.9 | 0.6 | 32.2 | 0.2 | 0.0 | 0.2 | 0.5 | 19.0 | 0.25 | 1.0 |
| pAT210-2 | 0.3 | 15.2 | 10.6 | 2.6 | 43.4 | 0.6 | 26.6 | 0.0 | 0.0 | 0.3 | 0.5 | 18.8 | 0.22 | 0.7 |

TABLE 22
Fatty acid composition of total fatty acid (TFA) content from selected Y. lipolytica
transformants with one of pAT207-212 in the ura3KO27-fad2KO1 strain. Except for
the ura3-fad2 controls, each row represents an individual transformant.

| | | | C16:1 | C16:1 | | | C18:1 | C18:1 |
|---|---|---|---|---|---|---|---|---|
| Transformant | C14:0 | C16:0 | Δ9 | Δ7 | C17:1 | C18:0 | Δ9 | Δ11 |
| ura3-fad2 | 0.2 | 14.5 | 5.5 | 1.6 | 0.8 | 15.2 | 56.5 | 0.3 |
| ura3-fad2 | 0.2 | 14.3 | 5.9 | 1.7 | 0.8 | 14.4 | 56.2 | 0.4 |
| ura3-fad2 | 0.2 | 13.8 | 6.2 | 2.0 | 0.9 | 12.6 | 57.8 | 0.4 |
| ura3-fad2 | 0.2 | 14.8 | 5.8 | 1.6 | 0.7 | 15.9 | 55.3 | 0.3 |
| pAT207-1 | 0.2 | 13.7 | 5.6 | 1.8 | 0.8 | 14.4 | 58.7 | 0.3 |
| pAT207-2 | 0.2 | 16.7 | 3.1 | 0.7 | 0.7 | 28.9 | 41.4 | 0.2 |
| pAT207-3 | 0.2 | 14.5 | 3.0 | 0.6 | 0.4 | 34.5 | 39.4 | 0.3 |
| pAT207-4 | 0.2 | 13.5 | 6.1 | 1.7 | 0.8 | 12.9 | 58.4 | 0.5 |
| pAT208-1 | 0.2 | 14.0 | 5.5 | 1.4 | 0.7 | 15.9 | 56.3 | 0.4 |
| pAT208-2 | 0.2 | 13.7 | 5.7 | 1.6 | 0.8 | 14.3 | 57.5 | 0.4 |
| pAT208-3 | 0.2 | 14.4 | 5.8 | 1.6 | 0.8 | 14.5 | 56.4 | 0.3 |
| pAT208-4 | 0.3 | 16.5 | 2.3 | 0.6 | 0.4 | 34.2 | 37.5 | 0.1 |
| pAT208-5 | 0.2 | 12.2 | 2.6 | 0.5 | 0.4 | 35.6 | 41.7 | 0.1 |
| pAT208-7 | 0.3 | 16.7 | 2.6 | 0.5 | 0.4 | 33.2 | 39.2 | 0.0 |
| pAT209-1 | 0.2 | 16.1 | 2.6 | 0.7 | 0.5 | 31.0 | 42.1 | 0.0 |
| pAT209-2 | 0.2 | 16.6 | 2.6 | 0.7 | 0.6 | 31.1 | 39.0 | 0.2 |
| pAT209-3 | 0.2 | 17.0 | 2.6 | 0.6 | 0.5 | 31.5 | 40.3 | 0.0 |
| pAT209-4 | 0.2 | 12.8 | 4.6 | 1.2 | 0.6 | 20.0 | 54.9 | 0.3 |
| pAT210-1 | 0.1 | 17.3 | 8.5 | 1.8 | 1.7 | 10.4 | 53.9 | 0.5 |
| pAT210-2 | 0.2 | 11.6 | 7.1 | 2.2 | 1.0 | 7.3 | 64.7 | 0.6 |
| pAT210-3 | 0.2 | 12.3 | 6.5 | 1.9 | 1.8 | 8.1 | 63.3 | 0.6 |
| pAT210-4 | 0.2 | 13.8 | 5.0 | 1.4 | 1.0 | 18.0 | 53.6 | 0.4 |
| pAT210-5 | 0.4 | 16.9 | 2.6 | 0.7 | 0.5 | 32.7 | 36.7 | 0.2 |
| pAT210-6 | 0.3 | 16.9 | 2.6 | 0.6 | 0.4 | 34.0 | 36.4 | 0.2 |
| pAT211-1 | 0.6 | 21.2 | 2.2 | 0.6 | 0.3 | 36.9 | 30.1 | 0.1 |
| pAT211-2 | 0.7 | 22.8 | 2.2 | 0.6 | 0.3 | 36.9 | 28.8 | 0.1 |
| pAT211-3 | 0.3 | 18.7 | 2.4 | 0.6 | 0.4 | 36.8 | 35.4 | 0.1 |
| pAT211-4 | 0.2 | 16.0 | 5.1 | 1.2 | 0.5 | 18.0 | 53.8 | 0.4 |
| pAT211-5 | 0.6 | 23.6 | 2.6 | 0.5 | 0.3 | 35.9 | 29.8 | 0.1 |
| pAT211-6 | 0.2 | 17.1 | 6.0 | 1.7 | 0.6 | 13.2 | 56.5 | 0.5 |
| pAT211-7 | 0.5 | 19.2 | 1.9 | 0.6 | 0.3 | 38.3 | 32.2 | 0.1 |
| pAT211-8 | 0.2 | 16.9 | 5.3 | 1.8 | 0.4 | 16.6 | 52.1 | 0.2 |
| pAT211-9 | 0.6 | 21.3 | 2.1 | 0.6 | 0.3 | 39.0 | 28.3 | 0.1 |
| pAT211-10 | 0.5 | 23.0 | 2.1 | 0.5 | 0.3 | 38.9 | 27.7 | 0.1 |
| pAT212-1 | 0.2 | 10.8 | 6.1 | 3.0 | 0.9 | 6.4 | 67.5 | 0.6 |
| pAT212-2 | 0.8 | 21.6 | 2.6 | 0.7 | 0.3 | 34.8 | 32.6 | 0.1 |
| pAT212-3 | 0.4 | 15.5 | 5.3 | 1.1 | 0.7 | 22.0 | 46.7 | 0.3 |
| pAT212-4 | 0.3 | 11.8 | 9.0 | 1.7 | 1.3 | 6.2 | 63.7 | 0.6 |
| pAT212-5 | 0.4 | 17.9 | 3.0 | 0.5 | 0.3 | 35.4 | 35.1 | 0.1 |
| pAT212-6 | 0.6 | 20.0 | 3.4 | 0.6 | 0.3 | 34.4 | 32.7 | 0.0 |
| pAT212-7 | 0.6 | 19.2 | 3.3 | 0.5 | 0.3 | 34.7 | 33.5 | 0.1 |

| | | | | | | L/S- | Lipid |
|---|---|---|---|---|---|---|---|
| | C18:2 | | | | Total | SFA | content |
| Transformant | Δ9, 12 | C20:0 | C22:0 | C24:0 | SFA | ratio | (% dcw) |
| ura3-fad2 | 0.1 | 0.8 | 0.5 | 3.5 | 34.7 | 1.37 | 11.9 |
| ura3-fad2 | 0.1 | 0.8 | 0.5 | 4.4 | 34.6 | 1.39 | 11.5 |
| ura3-fad2 | 0.1 | 0.8 | 0.5 | 4.1 | 31.9 | 1.28 | 10.6 |
| ura3-fad2 | 0.0 | 0.8 | 0.5 | 3.5 | 35.7 | 1.39 | 12.6 |
| pAT207-1 | 0.1 | 0.8 | 0.5 | 2.7 | 32.3 | 1.33 | 13.0 |
| pAT207-2 | 0.1 | 2.0 | 2.0 | 3.7 | 53.6 | 2.16 | 21.0 |
| pAT207-3 | 0.1 | 2.2 | 1.9 | 2.7 | 56.0 | 2.81 | 28.8 |
| pAT207-4 | 0.1 | 0.8 | 0.5 | 4.1 | 32.0 | 1.34 | 11.8 |
| pAT208-1 | 0.1 | 0.9 | 0.5 | 3.8 | 35.2 | 1.49 | 12.3 |
| pAT208-2 | 0.1 | 0.8 | 0.5 | 3.9 | 33.4 | 1.41 | 11.9 |
| pAT208-3 | 0.1 | 0.8 | 0.5 | 4.2 | 34.6 | 1.37 | 11.7 |
| pAT208-4 | 0.0 | 2.5 | 2.4 | 2.9 | 58.7 | 2.51 | 33.0 |
| pAT208-5 | 0.1 | 2.0 | 1.8 | 2.5 | 54.3 | 3.41 | 23.3 |
| pAT208-7 | 0.0 | 2.3 | 2.1 | 2.3 | 56.9 | 2.35 | 29.5 |
| pAT209-1 | 0.1 | 2.0 | 2.0 | 2.5 | 53.7 | 2.30 | 29.6 |
| pAT209-2 | 0.1 | 2.2 | 2.1 | 3.0 | 55.2 | 2.29 | 28.6 |
| pAT209-3 | 0.0 | 2.2 | 2.1 | 2.6 | 55.6 | 2.23 | 28.6 |
| pAT209-4 | 0.1 | 0.9 | 0.6 | 3.5 | 38.0 | 1.93 | 14.6 |
| pAT210-1 | 0.1 | 0.6 | 0.3 | 4.1 | 32.9 | 0.89 | 15.2 |
| pAT210-2 | 0.1 | 0.6 | 0.4 | 3.5 | 23.7 | 1.00 | 9.6 |
| pAT210-3 | 0.1 | 0.6 | 0.4 | 3.5 | 25.1 | 1.00 | 9.4 |
| pAT210-4 | 0.1 | 1.4 | 1.0 | 3.6 | 38.0 | 1.71 | 11.9 |
| pAT210-5 | 0.0 | 3.1 | 2.9 | 2.8 | 58.9 | 2.40 | 23.9 |
| pAT210-6 | 0.0 | 2.8 | 2.6 | 2.8 | 59.5 | 2.45 | 27.3 |
| pAT211-1 | 0.0 | 2.9 | 2.0 | 2.8 | 66.4 | 2.05 | 27.1 |
| pAT211-2 | 0.0 | 2.9 | 1.9 | 2.4 | 67.6 | 1.88 | 24.9 |
| pAT211-3 | 0.1 | 2.1 | 1.3 | 1.7 | 60.7 | 2.20 | 32.1 |
| pAT211-4 | 0.1 | 0.9 | 0.6 | 2.9 | 38.6 | 1.38 | 27.3 |
| pAT211-5 | 0.0 | 2.4 | 1.5 | 2.1 | 66.2 | 1.74 | 28.5 |
| pAT211-6 | 0.0 | 0.8 | 0.6 | 2.5 | 34.3 | 0.99 | 22.6 |
| pAT211-7 | 0.1 | 2.5 | 1.6 | 2.2 | 64.4 | 2.26 | 28.0 |
| pAT211-8 | 0.1 | 1.0 | 1.0 | 4.1 | 39.7 | 1.32 | 23.6 |
| pAT211-9 | 0.0 | 3.0 | 2.0 | 2.4 | 68.3 | 2.12 | 29.8 |
| pAT211-10 | 0.0 | 2.7 | 1.6 | 2.2 | 68.9 | 1.93 | 26.3 |
| pAT212-1 | 0.1 | 0.5 | 0.3 | 3.1 | 21.3 | 0.93 | 12.5 |
| pAT212-2 | 0.1 | 2.5 | 1.3 | 2.1 | 63.2 | 1.82 | 27.2 |
| pAT212-3 | 0.1 | 1.7 | 1.1 | 4.6 | 45.2 | 1.85 | 15.4 |
| pAT212-4 | 0.1 | 0.6 | 0.3 | 3.4 | 22.6 | 0.87 | 11.1 |
| pAT212-5 | 0.1 | 2.4 | 1.8 | 2.7 | 60.6 | 2.32 | 30.9 |
| pAT212-6 | 0.0 | 2.6 | 2.0 | 2.7 | 62.4 | 2.02 | 23.2 |
| pAT212-7 | 0.1 | 2.5 | 1.8 | 3.1 | 61.8 | 2.12 | 28.6 |

TABLE 23
Fatty acid composition of TAG content from selected Y. lipolytica transformants
with one of pAT207-212 in the ura3KO27-fad2KO1 strain. Except for the ura3-fad2
controls, each row represents an individual transformant.

| | | | C16:1 | C16:1 | | | C18:1 | C18:1 |
|---|---|---|---|---|---|---|---|---|
| Transformant | C14:0 | C16:0 | Δ9 | Δ7 | C17:1 | C18:0 | Δ9 | Δ11 |
| ura3-fad2 | 0.2 | 15.3 | 3.7 | 1.4 | 0.5 | 21.5 | 51.2 | 0.3 |
| ura3-fad2 | 0.2 | 15.1 | 4.1 | 1.5 | 0.5 | 21.1 | 51.1 | 0.4 |
| ura3-fad2 | 0.2 | 14.6 | 4.6 | 1.8 | 0.6 | 19.1 | 52.5 | 0.4 |
| ura3-fad2 | 0.2 | 15.4 | 4.0 | 1.3 | 0.5 | 22.2 | 50.4 | 0.3 |
| pAT207-1 | 0.2 | 13.8 | 3.9 | 1.5 | 0.5 | 21.7 | 52.3 | 0.3 |
| pAT207-2 | 0.3 | 15.7 | 2.4 | 0.8 | 0.5 | 30.1 | 43.4 | 0.2 |
| pAT207-3 | 0.2 | 13.3 | 2.6 | 0.6 | 0.3 | 32.0 | 44.7 | 0.2 |
| pAT207-4 | 0.2 | 13.8 | 4.6 | 1.6 | 0.5 | 18.5 | 54.5 | 0.5 |
| pAT208-1 | 0.2 | 14.5 | 3.8 | 1.2 | 0.5 | 22.1 | 51.8 | 0.3 |
| pAT208-2 | 0.2 | 14.2 | 4.1 | 1.5 | 0.5 | 20.7 | 52.7 | 0.4 |
| pAT208-3 | 0.2 | 15.2 | 4.1 | 1.5 | 0.5 | 20.6 | 51.6 | 0.3 |
| pAT208-4 | 0.3 | 13.8 | 2.0 | 0.7 | 0.4 | 31.7 | 43.7 | 0.1 |
| pAT208-5 | 0.1 | 11.7 | 1.9 | 0.5 | 0.3 | 34.4 | 45.1 | 0.0 |
| pAT208-7 | 0.3 | 14.3 | 2.2 | 0.6 | 0.3 | 31.8 | 43.9 | 0.0 |
| pAT209-1 | 0.2 | 14.1 | 2.2 | 0.7 | 0.4 | 28.8 | 47.2 | 0.0 |
| pAT209-2 | 0.2 | 14.5 | 2.2 | 0.7 | 0.6 | 29.6 | 45.2 | 0.1 |
| pAT209-3 | 0.2 | 14.9 | 2.2 | 0.6 | 0.4 | 30.6 | 44.6 | 0.0 |
| pAT209-4 | 0.2 | 12.5 | 3.3 | 1.0 | 0.4 | 24.3 | 53.3 | 0.3 |
| pAT210-1 | 0.2 | 18.2 | 6.9 | 1.7 | 1.2 | 13.5 | 53.6 | 0.4 |
| pAT210-2 | 0.2 | 12.0 | 5.7 | 2.1 | 0.6 | 13.5 | 58.8 | 0.7 |
| pAT210-3 | 0.2 | 13.6 | 4.8 | 1.8 | 1.1 | 14.8 | 56.4 | 0.7 |
| pAT210-4 | 0.2 | 14.1 | 3.6 | 1.4 | 0.6 | 24.1 | 48.8 | 0.4 |
| pAT210-5 | 0.4 | 13.8 | 2.3 | 0.8 | 0.4 | 33.3 | 40.7 | 0.2 |
| pAT210-6 | 0.3 | 13.6 | 2.2 | 0.6 | 0.4 | 33.7 | 40.9 | 0.2 |
| pAT211-1 | 0.5 | 15.0 | 2.2 | 0.8 | 0.3 | 35.2 | 37.4 | 0.1 |
| pAT211-2 | 0.6 | 15.6 | 2.4 | 0.8 | 0.3 | 35.2 | 37.0 | 0.1 |
| pAT211-3 | 0.3 | 15.2 | 2.4 | 0.6 | 0.4 | 34.3 | 41.5 | 0.1 |
| pAT211-4 | 0.2 | 15.8 | 4.3 | 1.1 | 0.4 | 19.0 | 54.6 | 0.3 |
| pAT211-5 | 0.6 | 18.3 | 3.1 | 0.8 | 0.3 | 33.0 | 38.2 | 0.1 |
| pAT211-6 | 0.2 | 16.7 | 5.2 | 1.6 | 0.5 | 13.8 | 57.6 | 0.4 |
| pAT211-7 | 0.5 | 13.8 | 2.0 | 0.7 | 0.3 | 37.4 | 38.7 | 0.1 |
| pAT211-8 | 0.2 | 16.8 | 4.4 | 1.7 | 0.3 | 17.9 | 53.2 | 0.2 |
| pAT211-9 | 0.5 | 13.9 | 2.4 | 0.8 | 0.3 | 36.4 | 37.2 | 0.1 |
| pAT211-10 | 0.5 | 15.4 | 2.5 | 0.7 | 0.3 | 35.7 | 37.3 | 0.1 |
| pAT212-1 | 0.2 | 9.0 | 5.0 | 2.8 | 0.6 | 10.0 | 67.7 | 0.7 |
| pAT212-2 | 0.6 | 15.1 | 2.7 | 0.8 | 0.4 | 32.7 | 40.7 | 0.2 |
| pAT212-3 | 0.4 | 14.0 | 4.4 | 1.2 | 0.5 | 28.1 | 43.5 | 0.3 |
| pAT212-4 | 0.3 | 13.7 | 6.8 | 1.9 | 0.8 | 14.1 | 54.8 | 0.7 |
| pAT212-5 | 0.4 | 15.2 | 2.9 | 0.6 | 0.3 | 34.9 | 39.1 | 0.1 |
| pAT212-6 | 0.6 | 15.1 | 3.5 | 0.8 | 0.3 | 33.9 | 37.7 | 0.1 |
| pAT212-7 | 0.5 | 14.7 | 3.1 | 0.6 | 0.3 | 34.2 | 38.5 | 0.1 |

| | | | | | | L/S- | TAG |
|---|---|---|---|---|---|---|---|
| | C18:2 | | | | Total | SFA | content |
| Transformant | Δ9, 12 | C20:0 | C22:0 | C24:0 | SFA | ratio | (% dcw) |
| ura3-fad2 | 0.1 | 0.9 | 0.8 | 3.9 | 42.5 | 1.75 | 4.0 |
| ura3-fad2 | 0.1 | 0.9 | 0.8 | 3.9 | 41.9 | 1.74 | 3.7 |
| ura3-fad2 | 0.1 | 0.9 | 0.8 | 4.0 | 39.5 | 1.67 | 3.2 |
| ura3-fad2 | 0.1 | 0.9 | 0.8 | 3.5 | 43.0 | 1.76 | 3.4 |
| pAT207-1 | 0.1 | 0.9 | 0.8 | 3.5 | 40.8 | 1.92 | 2.7 |
| pAT207-2 | 0.1 | 2.1 | 1.6 | 2.6 | 52.4 | 2.29 | 6.5 |
| pAT207-3 | 0.1 | 2.0 | 1.6 | 2.2 | 51.2 | 2.81 | 8.9 |
| pAT207-4 | 0.1 | 0.8 | 0.7 | 3.6 | 37.7 | 1.69 | 2.9 |
| pAT208-1 | 0.1 | 0.9 | 0.8 | 3.4 | 41.9 | 1.85 | 3.4 |
| pAT208-2 | 0.1 | 0.9 | 0.8 | 3.6 | 40.3 | 1.80 | 3.4 |
| pAT208-3 | 0.1 | 1.0 | 0.8 | 3.8 | 41.5 | 1.69 | 2.9 |
| pAT208-4 | 0.0 | 2.5 | 2.2 | 2.3 | 52.8 | 2.74 | 8.2 |
| pAT208-5 | 0.1 | 1.9 | 1.6 | 2.1 | 51.8 | 3.36 | 10.0 |
| pAT208-7 | 0.0 | 2.3 | 1.9 | 2.1 | 52.7 | 2.61 | 12.8 |
| pAT209-1 | 0.0 | 1.9 | 1.8 | 2.2 | 49.2 | 2.43 | 12.5 |
| pAT209-2 | 0.1 | 2.2 | 1.9 | 2.4 | 50.8 | 2.45 | 10.2 |
| pAT209-3 | 0.0 | 2.2 | 1.9 | 2.2 | 51.9 | 2.43 | 11.3 |
| pAT209-4 | 0.1 | 1.0 | 0.8 | 2.6 | 41.4 | 2.27 | 4.9 |
| pAT210-1 | 0.1 | 0.6 | 0.5 | 2.7 | 35.6 | 0.94 | 5.3 |
| pAT210-2 | 0.1 | 0.8 | 0.8 | 4.0 | 31.3 | 1.57 | 2.4 |
| pAT210-3 | 0.1 | 0.8 | 0.7 | 4.1 | 34.3 | 1.49 | 2.2 |
| pAT210-4 | 0.1 | 1.5 | 1.2 | 3.4 | 44.6 | 2.11 | 3.0 |
| pAT210-5 | 0.0 | 3.2 | 2.3 | 2.1 | 55.2 | 2.88 | 12.0 |
| pAT210-6 | 0.0 | 3.0 | 2.3 | 2.4 | 55.4 | 2.97 | 9.9 |
| pAT211-1 | 0.0 | 3.1 | 2.2 | 2.7 | 58.8 | 2.79 | 5.1 |
| pAT211-2 | 0.1 | 3.2 | 1.8 | 2.4 | 58.8 | 2.63 | 6.7 |
| pAT211-3 | 0.1 | 2.1 | 1.3 | 1.4 | 54.6 | 2.53 | 13.6 |
| pAT211-4 | 0.0 | 0.9 | 0.6 | 2.3 | 38.9 | 1.42 | 12.7 |
| pAT211-5 | 0.0 | 2.2 | 1.2 | 1.5 | 56.9 | 2.01 | 8.6 |
| pAT211-6 | 0.0 | 0.7 | 0.6 | 2.3 | 34.3 | 1.02 | 13.5 |
| pAT211-7 | 0.0 | 2.6 | 1.5 | 2.0 | 57.8 | 3.04 | 9.9 |
| pAT211-8 | 0.0 | 1.0 | 1.0 | 2.9 | 39.9 | 1.34 | 9.1 |
| pAT211-9 | 0.0 | 3.2 | 2.0 | 2.7 | 58.7 | 3.08 | 8.5 |
| pAT211-10 | 0.1 | 2.8 | 1.7 | 2.5 | 58.6 | 2.70 | 3.3 |
| pAT212-1 | 0.1 | 0.5 | 0.5 | 2.4 | 22.7 | 1.47 | 4.6 |
| pAT212-2 | 0.1 | 2.6 | 1.5 | 2.2 | 54.7 | 2.48 | 2.8 |
| pAT212-3 | 0.1 | 1.9 | 1.4 | 3.7 | 49.5 | 2.44 | 5.6 |
| pAT212-4 | 0.1 | 0.8 | 0.8 | 4.6 | 34.2 | 1.45 | 3.5 |
| pAT212-5 | 0.0 | 2.3 | 1.6 | 2.4 | 56.6 | 2.64 | 11.0 |
| pAT212-6 | 0.0 | 2.7 | 1.9 | 2.8 | 57.0 | 2.63 | 10.7 |
| pAT212-7 | 0.1 | 2.6 | 1.9 | 2.9 | 56.8 | 2.73 | 5.2 |

TABLE 24
Fatty acid composition of polar lipids from selected Y. lipolytica transformants
with one of pAT207-212 in the ura3KO27-fad2KO1 strain. Except for the ura3-fad2
controls, each row represents an individual transformant.

| | | | C16:1 | C16:1 | | | C18:1 | C18:1 |
|---|---|---|---|---|---|---|---|---|
| Transformant | C14:0 | C16:0 | Δ9 | Δ7 | C17:1 | C18:0 | Δ9 | Δ11 |
| ura3-fad2 | 0.1 | 8.6 | 7.4 | 1.8 | 1.3 | 1.5 | 77.9 | 0.0 |
| ura3-fad2 | 0.1 | 9.0 | 7.7 | 1.8 | 1.2 | 1.6 | 77.3 | 0.0 |
| ura3-fad2 | 0.1 | 8.6 | 7.8 | 2.1 | 1.3 | 1.4 | 77.4 | 0.0 |
| ura3-fad2 | 0.1 | 8.8 | 8.0 | 1.9 | 1.1 | 1.3 | 77.4 | 0.0 |
| pAT207-1 | 0.2 | 8.9 | 7.1 | 1.8 | 1.2 | 1.8 | 77.9 | 0.0 |
| pAT207-2 | 0.2 | 14.9 | 5.7 | 0.8 | 1.2 | 9.6 | 66.1 | 0.0 |
| pAT207-3 | 0.2 | 13.6 | 6.3 | 0.9 | 0.9 | 10.9 | 65.7 | 0.4 |
| pAT207-4 | 0.1 | 8.8 | 7.6 | 1.7 | 1.1 | 1.3 | 77.3 | 0.3 |
| pAT208-1 | 0.1 | 8.6 | 7.5 | 1.4 | 1.1 | 1.2 | 78.5 | 0.3 |
| pAT208-2 | 0.1 | 8.5 | 7.4 | 1.6 | 1.2 | 1.1 | 78.5 | 0.0 |
| pAT208-3 | 0.1 | 8.7 | 7.6 | 1.5 | 1.2 | 1.1 | 78.5 | 0.0 |
| pAT208-4 | 0.3 | 19.6 | 3.6 | 0.6 | 0.6 | 25.4 | 45.7 | 0.2 |
| pAT208-5 | 0.1 | 9.5 | 7.1 | 0.8 | 1.1 | 2.8 | 75.6 | 0.3 |
| pAT208-7 | 0.1 | 18.2 | 5.1 | 0.6 | 0.7 | 18.7 | 54.6 | 0.2 |
| pAT209-1 | 0.2 | 14.6 | 5.6 | 1.0 | 1.0 | 14.0 | 62.0 | 0.3 |
| pAT209-2 | 0.2 | 15.5 | 5.4 | 1.0 | 1.2 | 14.0 | 60.9 | 0.2 |
| pAT209-3 | 0.2 | 16.5 | 5.6 | 0.8 | 0.9 | 14.0 | 60.0 | 0.3 |
| pAT209-4 | 0.1 | 8.5 | 6.8 | 1.3 | 1.0 | 1.8 | 78.5 | 0.3 |
| pAT210-1 | 0.1 | 9.7 | 10.1 | 1.8 | 2.4 | 1.4 | 72.0 | 0.4 |
| pAT210-2 | 0.2 | 8.0 | 7.6 | 2.1 | 1.4 | 1.0 | 77.8 | 0.4 |
| pAT210-3 | 0.2 | 7.7 | 7.3 | 1.7 | 2.6 | 0.9 | 78.1 | 0.4 |
| pAT210-4 | 0.2 | 9.5 | 6.7 | 1.5 | 1.7 | 2.9 | 75.9 | 0.3 |
| pAT210-5 | 0.6 | 22.5 | 3.8 | 0.6 | 0.7 | 20.4 | 49.0 | 0.2 |
| pAT210-6 | 0.4 | 21.1 | 4.1 | 0.5 | 0.6 | 23.1 | 47.7 | 0.2 |
| pAT211-1 | 0.7 | 25.3 | 2.0 | 0.5 | 0.3 | 33.4 | 32.4 | 0.1 |
| pAT211-2 | 0.7 | 26.9 | 2.6 | 0.5 | 0.4 | 27.5 | 39.0 | 0.0 |
| pAT211-3 | 0.3 | 24.1 | 2.5 | 0.6 | 0.5 | 29.3 | 40.5 | 0.1 |
| pAT211-4 | 0.1 | 10.1 | 6.8 | 1.4 | 0.7 | 2.1 | 76.4 | 0.4 |
| pAT211-5 | 0.6 | 27.6 | 2.9 | 0.4 | 0.3 | 29.8 | 36.1 | 0.1 |
| pAT211-6 | 0.1 | 10.1 | 8.1 | 2.6 | 0.8 | 1.5 | 74.2 | 0.4 |
| pAT211-7 | 0.6 | 24.8 | 2.1 | 0.5 | 0.4 | 29.3 | 40.3 | 0.1 |
| pAT211-8 | 0.1 | 9.4 | 7.6 | 2.5 | 0.7 | 3.4 | 74.2 | 0.3 |
| pAT211-9 | 0.7 | 26.5 | 2.2 | 0.4 | 0.3 | 33.0 | 34.2 | 0.1 |
| pAT211-10 | 0.5 | 23.8 | 2.1 | 0.5 | 0.3 | 35.9 | 30.9 | 0.1 |
| pAT212-1 | 0.2 | 9.4 | 6.8 | 2.9 | 1.1 | 2.2 | 75.5 | 0.5 |
| pAT212-2 | 0.8 | 22.0 | 2.5 | 0.7 | 0.3 | 32.1 | 36.2 | 0.1 |
| pAT212-3 | 0.4 | 14.6 | 6.2 | 1.1 | 1.0 | 10.6 | 64.0 | 0.2 |
| pAT212-4 | 0.2 | 8.0 | 9.7 | 1.6 | 1.6 | 1.0 | 75.6 | 0.4 |
| pAT212-5 | 0.4 | 21.4 | 4.0 | 0.5 | 0.5 | 20.7 | 50.3 | 0.1 |
| pAT212-6 | 0.6 | 24.8 | 3.6 | 0.5 | 0.4 | 26.0 | 41.5 | 0.1 |
| pAT212-7 | 0.6 | 20.1 | 3.5 | 0.5 | 0.3 | 29.4 | 39.9 | 0.1 |

| | | | | | | | Polar |
|---|---|---|---|---|---|---|---|
| | | | | | | L/S- | lipid |
| | C18:2 | | | | Total | SFA | content |
| Transformant | Δ9, 12 | C20:0 | C22:0 | C24:0 | SFA | ratio | (% dcw) |
| ura3-fad2 | 0.0 | 0.3 | 0.2 | 0.3 | 11.3 | 0.27 | 2.3 |
| ura3-fad2 | 0.0 | 0.3 | 0.2 | 0.3 | 11.7 | 0.26 | 2.2 |
| ura3-fad2 | 0.0 | 0.4 | 0.2 | 0.3 | 11.3 | 0.26 | 2.3 |
| ura3-fad2 | 0.0 | 0.4 | 0.2 | 0.2 | 11.3 | 0.24 | 2.2 |
| pAT207-1 | 0.0 | 0.3 | 0.2 | 0.3 | 11.9 | 0.28 | 1.9 |
| pAT207-2 | 0.0 | 0.4 | 0.2 | 0.3 | 25.8 | 0.69 | 2.2 |
| pAT207-3 | 0.1 | 0.4 | 0.1 | 0.3 | 25.6 | 0.83 | 2.3 |
| pAT207-4 | 0.1 | 0.5 | 0.2 | 0.4 | 11.6 | 0.26 | 2.0 |
| pAT208-1 | 0.0 | 0.3 | 0.2 | 0.4 | 11.1 | 0.24 | 1.7 |
| pAT208-2 | 0.0 | 0.5 | 0.2 | 0.3 | 11.0 | 0.24 | 2.0 |
| pAT208-3 | 0.0 | 0.3 | 0.4 | 0.2 | 11.0 | 0.21 | 2.2 |
| pAT208-4 | 0.1 | 1.4 | 1.0 | 1.3 | 49.1 | 1.45 | 3.8 |
| pAT208-5 | 0.0 | 0.5 | 0.2 | 1.4 | 14.8 | 0.50 | 1.8 |
| pAT208-7 | 0.1 | 0.7 | 0.2 | 0.3 | 38.6 | 1.07 | 3.1 |
| pAT209-1 | 0.1 | 0.4 | 0.2 | 0.3 | 29.8 | 0.99 | 2.6 |
| pAT209-2 | 0.0 | 0.7 | 0.2 | 0.3 | 31.1 | 0.95 | 2.5 |
| pAT209-3 | 0.1 | 0.8 | 0.2 | 0.3 | 32.1 | 0.90 | 2.4 |
| pAT209-4 | 0.0 | 0.6 | 0.3 | 0.4 | 11.8 | 0.35 | 1.8 |
| pAT210-1 | 0.0 | 0.9 | 0.3 | 0.0 | 12.6 | 0.26 | 1.8 |
| pAT210-2 | 0.0 | 0.4 | 0.2 | 0.2 | 10.3 | 0.22 | 2.7 |
| pAT210-3 | 0.0 | 0.3 | 0.2 | 0.2 | 9.8 | 0.19 | 2.4 |
| pAT210-4 | 0.0 | 0.3 | 0.3 | 0.3 | 13.8 | 0.38 | 2.3 |
| pAT210-5 | 0.0 | 1.0 | 0.3 | 0.4 | 45.2 | 0.95 | 3.6 |
| pAT210-6 | 0.1 | 1.1 | 0.2 | 0.3 | 46.4 | 1.14 | 3.4 |
| pAT211-1 | 0.0 | 2.1 | 1.4 | 1.4 | 64.4 | 1.46 | 4.5 |
| pAT211-2 | 0.1 | 1.4 | 0.1 | 0.3 | 57.1 | 1.05 | 3.0 |
| pAT211-3 | 0.1 | 1.3 | 0.1 | 0.2 | 55.5 | 1.26 | 3.9 |
| pAT211-4 | 0.0 | 0.7 | 0.4 | 0.3 | 13.9 | 0.33 | 2.2 |
| pAT211-5 | 0.1 | 1.5 | 0.1 | 0.2 | 59.9 | 1.11 | 3.6 |
| pAT211-6 | 0.0 | 0.6 | 0.4 | 0.3 | 13.1 | 0.26 | 2.9 |
| pAT211-7 | 0.1 | 1.3 | 0.2 | 0.2 | 56.5 | 1.21 | 3.8 |
| pAT211-8 | 0.0 | 0.6 | 0.2 | 0.4 | 14.3 | 0.48 | 2.2 |
| pAT211-9 | 0.0 | 1.7 | 0.1 | 0.5 | 62.6 | 1.29 | 4.6 |
| pAT211-10 | 0.0 | 2.3 | 1.4 | 1.7 | 65.8 | 1.68 | 6.8 |
| pAT212-1 | 0.0 | 0.3 | 0.1 | 0.4 | 12.9 | 0.29 | 3.2 |
| pAT212-2 | 0.0 | 2.2 | 1.1 | 1.5 | 59.9 | 1.61 | 8.0 |
| pAT212-3 | 0.1 | 0.7 | 0.1 | 0.5 | 27.1 | 0.78 | 3.3 |
| pAT212-4 | 0.0 | 0.5 | 0.5 | 0.2 | 10.8 | 0.26 | 3.8 |
| pAT212-5 | 0.1 | 1.0 | 0.1 | 0.3 | 44.2 | 1.01 | 4.1 |
| pAT212-6 | 0.1 | 1.3 | 0.1 | 0.4 | 53.5 | 1.09 | 5.2 |
| pAT212-7 | 0.0 | 2.0 | 1.3 | 1.9 | 55.4 | 1.66 | 8.1 |

### Culturing Transformants in the Presence of Stearate

Selected transformants in the Y. lipolytica ura3 mutant strain, including transformant pAT209-5, and in the ura3-fad2 double mutant background, including transformants pAT211-03 and pAT211-09, are cultured in a defined medium with low nitrogen content which is supplemented with 10 g/L sodium stearate. In one experiment, the pH of the medium is adjusted to 8.0. After 4 days of culturing, cells are harvested from the cultures and lipids extracted. Analysis of the lipids shows that the amount of stearate reaches up to 75% or 85% by weight of the total fatty acid content of the extracted lipid, as well as in the TAG fraction of the lipid. When the stearate level is up to 75% by weight of the TFA content, the amount of oleic acid is at least 10% by weight of the TFA content. C20:0, C22:0 and C24:0 are also present, as are C16:1Δ7 and C17:1. In the lipid from the transformants in the ura3-fad2 strain, PUFA are either absent or present at a level of less than 0.2%. The extracted lipids also contain phospholipids which have an increased amount of SFA compared to lipids produced from the corresponding Y. lipolytica strain lacking the transgenes. The extracted lipids are solid at 25° C. These lipids are blended with oils such as a high oleic soy oil.

### Example 14. Further Modifications of Microbes to Increase Saturated Fatty Acid Content

In view of the experiments described in Example 13 with genetic constructs pAT207-212, the inventors wished to confirm the activities of the DGAT1 and FATA enzymes and the presumed lack of activity of the P. chlororaphis ACS enzymes encoded by those constructs by making single and double gene constructs. These constructs are compared to the triple gene construct pAT209 for their ability to increase SFA content and TFA content in Y. lipolytica.

The GoldenGate strategy was used to generate six constructs (Table 26), each also having a URA3 gene as selectable marker gene. Each construct was made by firstly, where necessary, modifying the 5′ upstream or 3′ downstream sequences of the three individual genes from Example 13 with flanking BsaI restriction sites to allow for cloning into the URA⁺ vector. For example, pAT120 having the MtDGAT1 coding region was made from pAT117 by PCR using primers at274 and at275, and pAT161 was made from pAT117 by PCR using primers at297 and at298. Assembly into the destination vector was done in analogous fashion to the construction of pAT207-212.

The construct pAT091 encoding the PcACS-X1 protein was assembled from components GG114, GG020, GG146 and the protein coding sequence from pAT021. The construct pAT108 encoding the GmFATA2 protein was assembled from components GG114, GG020, GG146 and the protein coding sequence from pAT067. The construct pAT135 encoding the MtDGAT1 protein was assembled from components GG114, GG020, GG146 and the protein coding sequence from pAT120. The construct pAT213 encoding the GmFATA2 and PcACS-X1 proteins was assembled from components GG114, GG146, GG151, GG014, GG021 and the protein coding sequences from pAT067 and pAT136. The construct pAT214 encoding the GmFATA2 and MtDGAT1 proteins was assembled from components GG114, GG146, GG151, GG014, GG021 and the protein coding sequences from pAT067 and pAT161. The construct pAT215 encoding the PcACS-X1 and MtDGAT1 proteins was assembled from components GG114, GG146, GG151, GG014, GG021 and the protein coding sequences from pAT021 and pAT161. Each genetic construct in the backbone vector GG114 was flanked by NotI restriction enzyme sites which allowed for excision of the DNA cassette with NotI. The nucleotide sequences of the NotI DNA fragments for these six constructs are provided as SEQ ID NOs:108 to 113 and of the oligonucleotide primers as SEQ ID NOs:100-107 (Table 25).

A seventh genetic construct (pAT216) was assembled by the GoldenGate strategy, having three genes encoding the GmFATA2, PcACS-X1 and Y. lipolytica DGA1 proteins along with a fourth gene which was the URA3 selectable marker gene. This construct was therefore identical to pAT209 except that the coding region for the macadamia MtDGAT1 was replaced with the Y. lipolytica DGA1 coding region. This was made since, as described in Example 11, the YlDGA1 protein was deduced to have specific activity for SFA and therefore was considered to be able to substitute for MtDGAT1 even though YlDGA1 was in the DGAT2 family. This construct pAT216 therefore allowed the comparison of the two DGAT enzymes in the context of the co-expression of the FATA thioesterase protein. To make pAT216, a first construct pAT162 having the YlDGA1 coding region was made by PCR using primers at299 and at300 (Table 25) and the protein coding sequence from Y. lipolytica. pAT216 was assembled using components from GG114, GG146, GG151, GG294, GG014, GG015, GG080 and the protein coding regions from pAT067, pAT136 and pAT162. The nucleotide sequence of the NotI DNA fragment from pAT216 is provided as SEQ ID NO: 114. The genetic constructs and their relevant component genes are listed in Table 26.

TABLE 25
Oligonucleotide primers used in this Example.

| | | SEQ |
|---|---|---|
| | | ID |
| Primer | Nucleotide sequence (5′ to 3′) | NO |
| at085 | GGGGGTCTCTACAAATGCGAGACTACGAGCATGTT | 100 |
| at086 | CCCGGTCTCTATCCCTATCCCAAGGCTTGCTGTTT | 101 |
| at274 | GGGGGTCTCTAATGCACAATGTCTATCTCTAACTCTC | 102 |
| at275 | CCCGGTCTCTTAGACTACTTAGAAACGCCGTTTTCC | 103 |
| at297 | GGGGGTCTCTACAACACAATGTCTATCTCTAACTCTC | 104 |
| at298 | CCCGGTCTCTATCCCTACTTAGAAACGCCGTTTTCC | 105 |
| at299 | GGGGGTCTCTCCACCACAATGACTATCGACTCACAAT | 106 |
| | AC | |
| at300 | CCCGGTCTCTATACTTACTCAATCATTCGGAACTCTG | 107 |

TABLE 26
Genetic constructs and component genes.

| Genetic | Gene | Gene | Gene | Gene | SEQ ID |
|---|---|---|---|---|---|
| construct | position 1 | position 2 | position 3 | position 4 | NO |
| pAT091 | Ura3 | PcACS-X1 | none | none | 108 |
| pAT108 | Ura3 | GmFATA2 | none | none | 109 |
| pAT135 | Ura3 | MtDGAT1 | none | none | 110 |
| pAT213 | Ura3 | GmFATA2 | PcACS-X1 | none | 111 |
| pAT214 | Ura3 | GmFATA2 | MtDGAT1 | none | 112 |
| pAT215 | Ura3 | PcACS-X1 | MtDGAT1 | none | 113 |
| pAT216 | Ura3 | GmFATA2 | PcACS-X1 | YIDGA1 | 114 |

Transformation of Y. lipolytica and Analysis of Lipids Produced by the Transformants

The NotI DNA fragments from these genetic constructs are used to transform the fad2KO1-ura3KO27 double mutant strain of Y. lipolytica, as described above for other constructs, selecting for transformant colonies on SD-ura plates incubated at 30° C. for 3 days. Genomic DNA is extracted from the putative transformants and the presence of the genomic integrated genetic constructs confirmed by PCR amplification. At least six confirmed transformant colonies from each transformation are selected and cultured in SD-ura medium overnight to produce starter cultures. The starter cultures are used to seed 10 ml DM-Glyc-LowN medium in 50 ml Falcon tubes to an OD600 of 0.2 and cultured for 96 h at 30° C. with shaking at 200 rpm for aeration. Lipid is extracted from harvested cells and analysed as described in Example 1. The fatty acid content and composition of the total fatty acid (TFA), the TAG and polar lipid fractions are analysed by GC of FAME.

### Example 15. Other Acyltransferase Genes

It was considered other fatty acyl acyltransferases as potential enzymes to increase saturated fatty acid content in microbes, in particular for increasing stearate and saturated fatty acids having at least 20 carbons: arachidic acid (C20:0), behenic acid (C22:0) and lignoceric acid (C24:0). The seeds of the tropical plant species Theobroma cacao are a source of lipid known as cocoa butter which has structured TAG molecules rich in stearic acid, used for example in chocolate and cosmetics. Eleven DGAT genes have been identified in the genome of Theobroma cacao (Wei et al., 2017). Two of the eleven DGAT enzymes, namely TcDGAT1 and TcDGAT2, were tested in S. cerevisiae for ability to produce cocoa butter-like TAG, but had little effect (Wei et al., 2017). The amino acid sequences for the DGATs are provided herein as SEQ ID NOs:115 to 125. T. cacao also has multiple genes encoding polypeptides with homology to GPAT enzymes, designated herein as TcGPAT1 to 13, and multiple genes encoding polypeptides with homology to PDAT enzymes, designated herein as TcPDAT1, 2 and 4-6. The amino acid sequences for the GPATs are provided herein as SEQ ID NOs:126 to 138 and the amino acid sequences for the PDATs are provided herein as SEQ ID NOs:139 to 143.

The present inventors noted that the TcDGAT9 to TcDGAT11 amino acid sequences were more related to the S. cerevisiae LRO1 gene product, which is a PDAT enzyme, than the other DGAT sequences from T. cacao. Of the candidate DGAT enzymes, the TcDGAT9, 10 and 11 enzymes were therefore of particular interest.

Codon optimised nucleotide sequences were designed to express the candidate DGAT, GPAT and PDAT enzymes in Y. lipolytica, either singly or in combination with a FATA enzyme, for example a GmFATA enzyme which is more active on stearoyl-ACP than palmitoyl-ACP. The nucleotide sequences are provided herein as SEQ ID NOs:144 to 154 for the DGATs, SEQ ID NOs:155 to 167 for the GPATs and SEQ ID NOs:168 to 172 for the candidate PDATs. The TcDGAT, TcGPAT and TcPDAT protein coding sequences were codon optimised for expression in Y. lipolytica. In each case, the codon optimised sequence was between 73% and 77% identical to the native protein coding sequence from T. cacao along the full length of the sequences i.e between 23% and 27% of the nucleotides were substituted by the codon optimisation.

Eleven genetic constructs were designed, each to express one of the TcDGAT enzymes together with a GmFATA enzyme, using the approach described in Example 14 for pAT214 except for substituting the MtDGAT sequence with a T. cacao sequence. Thirteen constructs are similarly designed for the GPATs and five constructs for the PDATs. Combinations of DGATs and GPATs, DGATs and PDATs, and GPATs and PDATs are also designed. The constructs are synthesised by the GoldenGate strategy in analogous fashion to other constructs as described above and in Example 1. Each construct is introduced into the ura3KO27-fad2KO1 mutant strain of Y. lipolytica. Confirmed transformants are cultured in a low nitrogen medium, lipids are extracted from the cells and the TFA, TAG and polar lipid fractions are analysed for content and fatty acid composition. At least some of the fatty acyl acyltransferases are expected to increase both the TAG content in the cells and the SFA content in the extracted lipid. Transformants with the desired fatty acid profiles are selected.

Codon optimised nucleotide sequences were also designed to express the candidate GPAT and PDAT enzymes in Y. lipolytica, either singly or in combination with a FATA enzyme, a DGAT enzyme, or both a FATA enzyme and a DGAT enzyme. Constructs were also designed to express two or three candidate PDAT enzymes in Y. lipolytica.

### Multi-Gene Vector Designs

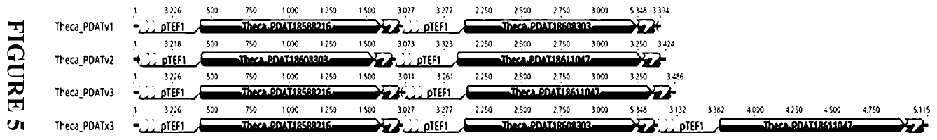

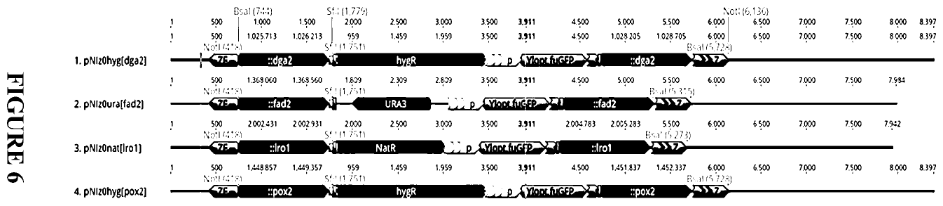

DNA sequences for open reading frames (ORFs) for three T. cacao PDATs genes, an acyl-ACP thioesterase from Garcinia mangostana (GmFATA1) and a LPAAT from M. alpina (Table 27) were codon optimised for expression in Y. lipolytica and genetic constructs chemically synthesised as multigene expression cassettes (FIG. 5). Each expression cassette was designed with flanking SfiI restriction enzyme sites to provide for the insertion into yeast integration vectors. Each gene was operably linked to the constitutive Y. lipolytica TEF1 promoter and terminated by the 3′ transcription termination/polyadenylation region of the Y. lipolytica LIP2 gene. The recipient integration vectors are shown schematically in FIG. 6. These integration vectors each contained a selectable marker gene which was a hygromycin resistance gene, a nourseothricin resistance gene or a URA3 gene for selection of transformants in Y. lipolytica. They also contained a GFP expression cassette as a reporter gene for easy detection of transformants, 5′- and 3′-Zeta-sequences for semi-random insertion if so desired, and 5′-upstream and 3′-downstream sequences from the DGA2, FAD2, LRO1 or POX2 genes of about 1,000 nucleotides each for targeted integration into the Y. lipolytica genome. Integration of the cassette into one of these genes would therefore simultaneously disrupt either the YlDGA2, YlFAD2, YlLRO1 or YlPOX2 genes when integration occurs into those genes. Vector and insert combinations are indicated in Table 28.

Each construct is introduced into the ura3KO27-fad2KO1 mutant strain of Y. lipolytica and into a FAD2 wild-type strain. Confirmed transformants are cultured in a low nitrogen medium, lipids are extracted from the cells and the TFA, TAG and polar lipid fractions are analysed for content and fatty acid composition. At least some of the fatty acyl acyltransferases are expected to increase both the TAG content in the cells and the SFA content in the extracted lipid. Transformants with the desired fatty acid profiles are selected.

TABLE 27
Enzymes that are tested for saturated fatty acid production.

| Gene name | Reported enzyme class | SEQ ID NO |
|---|---|---|
| GmFATA1 | acyl-ACP thioesterase | 83 |
| MaLPAAT | lysophosphatidic acid acyltransferase | 90 |
| TcPDAT5 | GDSL esterase/lipase 5 | 171 |
| TcPDAT1 | GDSL esterase/lipase 1 | 168 |
| TcPDAT2 | GDSL esterase/lipase | 169 |

TABLE 28
Cassette and vector combinations used.

| Cassette | Vector | Target/Marker |
|---|---|---|
| Garma_FATA1 + | PNIz0hyg[dga2] | Yarli_DGA2/hygR |
| Moral_LPAAT1 | (SEQ ID NO: 173) | |
| Theca_PDATx3 | PNIz0ura[fad2] | Yarli_FAD2/URA3 |
| | (SEQ ID NO: 174) | |
| Theca_PDATx3 | PNIz0nat[lro1] | Yarli_LRO1/NatR |
| | (SEQ ID NO: 175) | |
| Theca_PDATx3 | PNIz0hyg[pox2] | Yarli_POX2/hygR |
| | (SEQ ID NO: 176) | |
| Theca_PDATv1 | PNIz0ura[fad2] | Yarli_FAD2/URA3 |
| | (SEQ ID NO: 174) | |
| Theca_PDATv2 | PNIz0ura[fad2] | Yarli_FAD2/URA3 |
| | (SEQ ID NO: 174) | |
| Theca_PDATv3 | PNIz0ura[fad2] | Yarli_FAD2/URA3 |
| | (SEQ ID NO: 174) | |

### Example 16. Larger Scale Lipid Extraction to Produce Solid Fats

In order to test biomass production, recovery and lipid extraction processes from transgenic yeast cells grown in batch culture at larger scale, lipid was extracted from transformed Y. lipolytica strain yNI0056 after growth in a 25 L fermenter, as follows. The strain designated yNI0056 was selected from transformants of Y. lipolytica strain yNI0142 (ura3-fad2) using DNA of pAT207 (Example 13) containing genes encoding the GmFATA1, PcACS-X1 and MtDGAT1 polypeptides, selecting for URA+ transformants. Strain yNI0056 was selected after transformants with pAT207 were grown for 48 h in 10 ml of YPD medium in a 50 ml Falcon tube, where it produced TAG at 4.3% (w/dry weight) having 52.5% SFA including 21.6% stearic acid, 11.8% palmitic acid, 2.4% C20:0, 4.7% C22:0 and 11.6% C24:0, with an L/S-SFA ratio of 3.3.

An inoculum for the 20 L culture was prepared in a two-step process. In the first step, a single colony of yNI0056 was inoculated into 10 ml of YPD medium and cultured for 16 h at 30° C., shaking the culture at 250 rpm. The 10 ml culture was then inoculated into 1 L of YPD medium in a 2.5 L flask and cultured for 24 h at 30° C., shaking at 200 rpm, to produce the inoculum. The cell density after this second step was measured, with an OD of 25. A sample was examined by light microscopy to check for contamination. A Sartorius Stedim Biostat C Plus automated fermenter was washed with 4% NaOH solution to clean the vessel and tubes, and then washed with tap water. The temperature and pH probes were calibrated. Twenty litres of DM-Glyc-LowN medium (Example 1) containing 80 g/L glycerol as carbon source was added to the fermenter and sterilised in place by heating to 121° C. for 20 min, then cooled to 30° C. for inoculation. The 1 L inoculum was added for a first stage fermentation for biomass growth at high dissolved oxygen (DO) by controlling aeration at VVM=0.5 and agitation at 400 rpm for 24 h. VVM as used herein is the volumetric flow rate per unit volume of culture medium per min. After 24 h culturing, the air flow rate was reduced to VVM=0.05 (1 L/min) and agitation at 180 rpm to increase lipid synthesis, and cultured for a further 27 h. The temperature was maintained at 30° C. by using the fermenter chilling system. Foaming was controlled by adding silicone-based antifoam solution to a final concentration of 0.075 g/L.

The culture had reached maximum OD and cell dry weight at about 25 h of culturing (Table 29). At this time, the pH of the culture medium had decreased to 4.53, at which timepoint some 2 M NaOH was added. The pH increased to 5.70 after this addition. The desired pH was 6.0 but the fermenter pH meter was not working properly. The cells were harvested from the culture by centrifugation at 4,000 rpm for 5 min, with removal of the supernatant. The cell pellets were washed twice with water, each time recovering the cells by centrifugation, providing a total wet weight of 453 g which was equivalent to 114 g dry weight, i.e. 25% solids. The conversion rate of glycerol to biomass in this experiment was 13.1%, so lower than optimal.

To extract the lipid from the cells, 450 g cell biomass (wet weight) was resuspended in 2 L water and the suspension passed through an Emulsiflex C5 homogeniser. When an aliquot of the treated suspension was observed under light microscope, both whole cells and cell debris were visible, indicating that many cells were at least damaged. The treated suspension was centrifuged in VX 22G high-speed refrigerated centrifuge at 4,730 rcf at 4° C. for 20 min. The cell pellets were transferred to a 1 L beaker and resuspended in 600 mL ethanol with stirring for 10 min to dewater the cellular material. The mixture was centrifuged at 4,730 rcf at 4° C. for 20 min. The ethanol supernatant was collected. The cellular pellets were chalky/clay-like in consistency. They were transferred to a 2 L bottle and mixed with 1.5 L hexane with shaking at 120 rpm overnight to extract hexane-soluble lipids. The mixture was filtered under vacuum using a Buchner funnel. The hexane supernatant was collected in a round bottom flask and evaporated off using a rotary evaporator at a bath temperature of 35° C. with the vacuum set to 300 mbar. The extracted lipid was a viscous liquid at room temperature, yellowish in colour and translucent. The extracted material weighed 6.3 g in total.

TABLE 29
OD, cell dry weight and pH measured
throughout the culturing of yNI0056.

| Time (hrs) | OD | DW(g/L) | pH |
|---|---|---|---|
| 0 | 2 | 0 | 6.40 |
| 3 | 3.7 | 1.86 | 6.38 |
| 5.5 | 5.7 | 3.94 | 6.17 |
| 22 | 14.5 | 8.83 | 4.32 |
| 22.1 | 14.5 | 8.83 | 4.50 |
| 25 | 20.5 | 10.05 | 4.53 |
| 25.1 | 20.5 | 10.50 | 5.70 |
| 28 | 22 | 7.79 | 5.46 |
| 30 | 22 | 8.33 | 5.93 |
| 46 | 22.5 | 8.25 | 5.62 |
| 51 | 21.5 | 7.81 | 5.62 |

This sample is cooled to −20° C. at which temperature it is a frozen solid, and then transferred to 4° C. to thaw slowly. This allows larger, more saturated TAG molecules to crystallize out, providing a solid fat fraction with increased SFA content, particularly stearic acid as well as C20:0, C22:0 and C24:0. Alternatively, the extracted lipid can be fractionated on a silica column to obtain the TAG fraction that has increased SFA content.

Further Experiments with Large Scale Culturing (B014)

Culture conditions were modified in a following experiment at the 20 L scale. The same Y. lipolytica strain yNI0056 and culture medium was used, but the pH of the culture was better controlled at 6.0 and the temperature maintained at 28° C. The inoculum was prepared in the defined medium (DM-Glyc-LowN) as four 200 mL cultures in 500 mL flasks, cultured for 24 h to an OD600 of between 1.5 and 2.5 and introduced into the fermenter by overpressure. The measured culture density (OD600) at inoculation was 0.026. Culturing continued to 92 h post-inoculation. In stage 1 (0-48 h), the DO of at least 1 ppm (20% saturated) was maintained by increased stirring/agitation at 300-400 rpm with an airflow of 0.5-0.6 VVM and backpressure if the dissolved oxygen (DO) dropped below 1 ppm. In stage 2 (48 h to 92 h), more glycerol was added to about 60 g/L in the medium if the residual glycerol at any timepoint was less than 5 g/L. During the stage 1 of fermentation, the air pressure was kept between 5-10 PSI, the air flow between 10-13 L/min and DO decreased from 18.8 to 4.0 ppm 02. During stage 2 of fermentation (48-92 h), the pressure was kept around PSI, stirring/agitation at 100-150 rpm, the air flow around 5 L/min and the DO dropped from 4.0 to 0.9 at harvest, i.e more limited DO to increase TAG biosynthesis in stage 2. Samples were removed twice per day to assess OD600, glucose and citric acid levels by HPLC and refractive index, dry weight cell production and microscopic observation, nitrogen levels by Kjeldahl inorganic nitrogen test, phosphate reflectometric test strip and sulphate by colorimetric test strip. After this, the culture was heat treated to inactivate the yeast cells, cooled and the cells collected by centrifugation.

The OD increased from 5.1 at 24 h to 7.3 at 72 h and dropped to 6.9 at 96 h. The decrease in growth after 72 h may be attributed to low levels of nitrogen in the medium. Phosphate and sulphate were still available in the medium at 96 h. The citrate concentration dropped significantly at 24 h, suggesting assimilation of citrate during the growth phase, and then increased to 4.228 g/L at 96 hours post inoculation from 1.7 g/L at inoculation, consistent with a switch to more TAG synthesis. As indicated by DCW, OD600 and glycerol consumption (42.7%), the majority of yeast growth occurred during the first 30 h of cultivation, through cell division and then a slower accumulation occurred from 30 h to 96 h, presumably due to conversion of excess carbon to lipid. DCW increased from 5.5 g/L at 24 h to 7.0 g/L at 72 h and the dropped to 6.5 g/L at 96 h after heat treatment.

The cells were harvested by centrifugation, yielding 20.3 g of dry biomass. This was considered much less than optimal yield of biomass from the culture volume. Lipid was extracted from the cells into 40 ml of hexane with disruption of cells using the Ultraturrax at maximum speed for 10 min. The disruption step was repeated twice more, with stirring in between. The mixture was stirred overnight, after which it was centrifuged and the hexane supernatant collected. The solvent was removed using a rotary evaporator, yielding 1 g of dried, extracted lipid. This was dissolved in hexane to remove it from the evaporator flask. The hexane was partially evaporated overnight. Centrifugation of the material provided a liquid supernatant and a solid material which was soluble in chloroform. Both fractions contained considerable TAG as analysed by TLC. This extracted lipid is fractionated with acetone to increase the saturated fatty acid content.

Further Experiments with Large Scale Culturing (B016)

The cell yield in the experiment B014 was not optimal compared to B001; this may have been due to exhaustion of the nitrogen in the growth medium too early in the culturing. The culture medium was therefore adjusted in experiment B016 by increasing the nitrogen concentration to 2 g/L of ammonium phosphate rather than 0.5 g/L during the cell growth phase, seeking to increase biomass before nitrogen limitation and induction of more TAG synthesis. At the point at which growth was becoming limited by the nitrogen supply (65 h), a feed containing 60 g/L glycerol, plus magnesium sulphate, thiamine hydrochloride and trace elements at the same level as in the initial 20 L culture was added to the culture. The measured culture density (OD600) at inoculation was 0.097. As indicated by the dry weight of biomass, OD and glycerol consumption, most of the yeast growth occurred within the first 40.5 hours of cultivation by cell division and then a slower accumulation occurred from 40.5 h to 88.4 hours, presumably due to conversion of excess carbon to fat, to be confirmed by determining the fatty acid content, as well as depletion of nitrogen. Further addition of glycerol at 65 h didn't result in further increased dry weight of biomass and OD600, which might have been due to conversion of carbon to lipid when nitrogen was limited. The final OD600 was 20.6 and DW of 16.5 g/L. Samples for HPLC were filtered through a 0.22 μm filter. The citric acid concentration increased to 8.7 g/L at 88.4 hours post inoculation from 1.7 g/L at inoculation. In previous experiments, the accumulation of citric acid corresponded to the accumulation of lipid, especially TAG. Sulphate was present in considerable excess throughout the experiment. Phosphate was steadily consumed with 20% remaining at the end of the ferment, as was nitrogen. The increase of nitrogen and phosphate at the start of the experiment, by the increase in DAP concentration from 0.5 g/L to 2.0 g/L, resulted in increase in biomass in the first 24 hours to about 16 g/L as compared to 5.5 g/L in experiment B014. The initial OD of the culture was also higher (0.097) in this experiment than in B014 (0.05), but lower than that of B001 (0.132). It is considered that increasing the initial OD will result in more biomass in the growth phase and in turn more lipid in the second stage of fermentation.

The cell culture was sampled at 47.5 h, 65.2 h and at the completion of culturing at 88.4 h. At this time, the culture was heat treated at 105° C. for 15 min to kill the cells, cooled and the cells harvested by centrifugation. A viable cell count obtained by plating aliquots of the heat-treated culture showed that not all cells had been killed, with 105 viable cells per ml remaining i.e. 99.9% killing. The culture yielded a cell paste of 1000 g, having a dry weight after freeze drying of 220.9 g.

Lipid was extracted from 180 g of cells into 400 ml hexane, using the Ultraturrax to disrupt the cells as before. A solid lipid of 8.1 g was recovered after evaporation of the hexane. The lipid was dissolved in a small quantity of hexane to remove it from the evaporator flask, after warming to 50° C. collected it in a tube. After centrifugation, there appeared to be two layers. Both layers were collected and analysed separately. The hexane was evaporated from both samples, providing two lipid samples which were solid at room temperature. These were washed three times with cold ethanol and dried. This yielded extracted lipids of 7.3 g and 0.8 g in the first and second samples, respectively. Aliquots analysed by TLC showed the presence of mostly TAG with some polar lipids present as well.

The cell debris that remained from the first hexane extraction was also extracted further to obtain residual lipid. This lipid was also fractionated and the fatty acid composition of both the polar and TAG fractions from this “cell debris” lipid determined (Table 30).

Fatty acid analysis by GC showed that the saturated fatty acid content of the extracted lipid was 36% on a weight basis as a percentage of the total fatty acid content, predominantly made of palmitate at about 18% and stearate at about 12%, but also including all three of C20:0, C22:0 and C24:0 with about 4% C24:0. The SFA content and the stearate level were increased relative to corresponding data for the wild-type strain W29. The extracted lipids are fractionated by precipitation with acetone to increase the saturated fatty acid content.

For Table 30, the extracted lipids did not contain detectable amounts of C8:0, C10:0, C12:0, C14:1, C15:1, C18:1Δ11, C18:2ω6 (LA), C18:3ω3 (ALA) or other PUFA.

TABLE 30
Fatty acid composition of the polar and TAG fractions of extracted lipid and remaining
cell debris following extraction of Y. lipolytica strain yNI0056 cells.

| | Experiment B014 | | | | Experiment B016 | | | |
|---|---|---|---|---|---|---|---|---|
| | Cell | Cell | | | Cell | Cell | | |
| | Debris | Debris | Lipid: | Lipid: | Debris | Debris | Lipid: | Lipid: |
| Sample | Polar | TAG | Polar | TAG | Polar | TAG | Polar | TAG |
| C14:0 | 0.0 | 0.0 | 0.0 | 0.2 | 0.0 | 0.2 | 0.0 | 0.2 |
| C15:0 | 0.0 | 0.0 | 0.3 | 0.2 | 0.0 | 0.2 | 0.0 | 0.2 |
| C16:0 | 18.7 | 17.0 | 19.4 | 16.3 | 18.3 | 17.4 | 18.4 | 17.6 |
| C16:1 | 12.4 | 9.3 | 10.8 | 9.0 | 11.3 | 8.0 | 9.8 | 8.3 |
| C18:0 | 2.7 | 11.6 | 1.4 | 10.8 | 2.4 | 12.2 | 8.7 | 12.0 |
| C18:1Δ9 | 66.3 | 61.4 | 68.0 | 58.3 | 68.0 | 56.0 | 63.0 | 56.3 |
| C20:0 | 0.0 | 0.7 | 0.0 | 0.6 | 0.0 | 0.7 | 0.0 | 0.7 |
| C22:0 | 0.0 | 0.0 | 0.0 | 0.8 | 0.0 | 1.1 | 0.0 | 0.9 |
| C24:0 | 0.0 | 0.0 | 0.0 | 3.6 | 0.0 | 4.1 | 0.0 | 3.8 |
| Total | 21.3 | 29.3 | 21.2 | 32.5 | 20.7 | 36.0 | 27.2 | 35.4 |
| SFA | | | | | | | | |

Further Experiments with Large Scale Culturing (B019)

The cell yield in the experiment B016 was much better compared to B0014 but the heat treatment was not fully effective in killing all cells at the termination of the culture. Therefore, experiment B0019 was carried out using similar conditions as in B0016, with one difference being that the culture was heat treated at 120° C. for 3 min rather than 105° C. The concentration of nitrogen was further increased by 50% to 3.0 g/L DAP and the feed with glycerol (excluding nitrogen) was introduced gradually to the fermenter starting at 24 h. The culture was sampled at 24 h, 40 h and 48 h timepoints and heat treated at 120° C. at the 60 h timepoint. After the culture was cooled to 10° C. in situ in the fermenter, it was noticed that considerable fat had deposited at the surface of the medium and on some of the baffles of the agitator. This deposit was collected by skimming the surface of the culture medium and from the baffles, yielding 3.6 g of creamy coloured fat. It was concluded that the heat treatment was disrupting many of the yeast cells, causing release of lipid into the culture medium.

Cell samples were examined by light microscopy to observe their morphology, both after the initial growth phase and at the end of culturing prior to the heat treatment. The majority of cells appeared to be elongated rather than oval shaped as typical of yeast, whereas other cells were either oval shaped or were intermediate in shape.

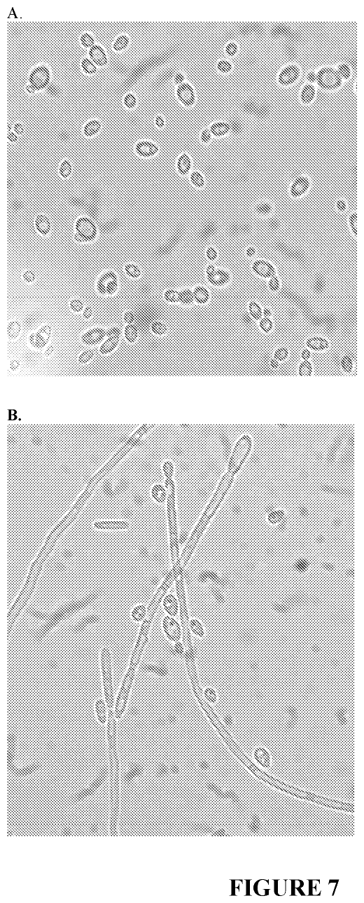

In another experiment, wild-type Y. lipolytica strain W29 and transformed strain yNI0056 were cultured under similar conditions and the cells observed by light microscopy. Many of the transformed cells were in a hyphal form, greatly elongated in shape, while others were either oval shaped or somewhat elongated. In contrast, none of the wild-type W29 cells showed the elongated or hyphal morphology (FIGS. 7A and 7B). Such cell morphology of the transformed cells was considered to be an indication of a stressed state of the cells, particularly caused at relatively low dissolved oxygen levels in the culture.

### Example 17. Lipid Fractionation

Crude lipid preparations may be fractionated with organic solvents to provide purer polar lipids or fractions having mostly neutral (non-polar) lipids including TAG (e.g. U.S. Pat. No. 7,550,616). For example, some reported methods use differential solubility of neutral and polar lipids in organic solvents such as ethanol or acetone. To test some of these methods, fractionation of several lipids having a mixture of substantial neutral and polar lipids was attempted, including egg yolk lipid and krill lipid, as model systems.

The lipids in chicken eggs are present mostly in the yolk fraction which constitutes about 33% lipid by weight. The lipids, which are closely associated with proteins in the yolk, are mostly TAG (66% by weight), with phospholipids (PL, 28%) and cholesterol and its esters (6%) present in lower amounts (Belitz et al., 2009). The PL contains some ω3 and ω6 fatty acids (Gladkowski et al., 2011). Based on the method of Palacios and Wang (2005), Gladkowski et al., (2012) extracted PL from egg yolk with ethanol and then purified the PL by removing neutral lipids by precipitation of the PL with cold acetone.

Fresh egg yolk (17 g), egg lecithin powder (20.4 g) and krill oil from Euphausia superba (17.7 g) obtained from commercially available krill oil capsules (Bioglan Red Krill Oil; Natural Bio Pty Ltd, Warriewood, NSW, Australia) were each mixed with 60 ml of ethanol and stirred for 30 min at room temperature. The mixture was centrifuged and the ethanol supernatant collected. The precipitate was extracted twice more, each time with 60 ml ethanol. The extraction mixtures were centrifuged again and the ethanol supernatants combined. Each precipitate was retained for extraction of neutral lipids. The ethanol from the combined supernatants was evaporated using a SR-100 rotary evaporator (Buchi, Switzerland) operating at 400 rpm with a vacuum of 15 mbar, with the chiller set at −16° C. and the waterbath at 37° C. This yielded 3.2 g of PL-enriched lipid extract from the 17 g of fresh egg yolk, 5.86 g from the 20.4 g of egg lecithin powder and 17.83 g of enriched PL recovered from the krill oil. The lipid recovered from the krill oil probably still contained a small amount of solvent. Nevertheless the recovery of essentially 100% indicated that the krill oil from the capsules was highly enriched for PL to begin with.

Aliquots of the recovered lipids were analysed by TLC as described in Example 1 using hexane:diethylether:acetic acid (70:30:1; v/v/v) as solvent. The ethanol extracts from fresh egg yolk and egg yolk lecithin powder were observed to contain substantial amounts of polar lipid as well as a small amount TAG, while the krill oil extract had no detected TAG.

To further purify the polar lipids from the fresh egg yolk, the dried extract was dissolved in 30 ml hexane and the solution cooled in an ice bath to 0° C. Next, 60 ml of cold acetone (−20° C.) was added to the solution to precipitate the PL for at least 20 min. Other experiments showed that more precipitate formed by keeping the mixtures at 0° C. overnight. The precipitate was collected and dried under vacuum. Samples of the lipid were dissolved in chloroform and analysed by TLC to estimate the polar lipid and TAG contents. The first precipitate was shown to have mostly polar lipid with some TAG. To further purify the polar lipid, the precipitate was washed 5 times with 20 ml portions of cold acetone (−20° C.) to remove more of the TAG and other neutral lipids. The residual solvent was removed from the precipitate by rotary evaporation at room temperature for 10 h. The lipid yield was measured gravimetrically and a small aliquot used for analysis of the fatty acid composition by GC quantitation of FAME. From the initial input of 17 g of fresh egg yolk, 1.1 gram of purified polar lipid was recovered. An aliquot of this extracted lipid was analysed by TLC and was observed to be essentially devoid of any neutral lipids, including TAG. These observations were consistent with those reported by Gladkowski et al., (2012) who found their extracts to be 96% pure PL.

Neutral lipid was extracted from the precipitates after the ethanol extraction of the egg yolk and egg yolk powder by extracting the precipitate twice with 50 ml of hexane. The combined hexane solution containing the neutral lipid was washed four times, each time with 50 ml of 90% ethanol. The hexane was then evaporated under reduced pressure to provide the purified neutral lipids from egg yolk.

To determine the fatty acid composition of the extracted lipids, the total fatty acids in aliquots were converted to FAME for GC analysis as described in Example 1. This included the samples (1^{st} ppt) after the ethanol extraction but before the hexane/acetone precipitation, as well as samples (2^{nd} ppt) after the hexane/acetone precipitation. The data is shown in Table 31. The ethanol-soluble lipid isolated from the fresh egg yolk and acetone precipitated lipid purified therefrom contained C16:0 and C18:0 as the main saturated fatty acids. The first lipid precipitate from fresh egg yolk containing 24.7% (C16:0) and 15.6% (C18:0) while the more purified polar lipid contained 27% (C16:0) and 16% (C18:0). The amount of LA in the 2^{nd} precipitate was slightly higher than in the 1^{st} precipitate; LA is present at greater amounts in PL than in TAG. Both fresh egg yolk and the purer polar lipid preparations also contained ω6 and ω3 LC-PUFA. For instance, the fresh egg yolk Is precipitate contained 5.3% C20:4 (ARA), 2.3% C20:5 (EPA) and 5% C22:6 (DHA) while more purified polar lipid preparation contained 5.3% ARA and 4% DHA. The first precipitate from the krill oil and the more purified polar lipid from the krill oil had C16:0 as their main saturated fatty acid. The krill oil 1^{st} precipitate and the more purified polar lipid also contained substantial amounts of 3 LC-PUFA, namely 1.10% ARA, 34.7% EPA and 19.0% DHA 15 in the 1^{st} precipitate, while the more purified polar lipid contained 1.1% ARA, 48.1% EPA and 25.7% DHA. The precipitated lipid from the egg yolk lecithin powder had 17% C16:0 and 4% C18:0 but was low in the LC-PUFA EPA and DHA. It was considered that the low LC-PUFA content of the lecithin powder was likely due to oxidative breakdown of those polyunsaturated fatty acids during its production or storage.

An alternative method to purify polar lipids by fractionation from a total lipid preparation is to use silica-based column chromatography such as, for example, use of SPE columns (HyperSep aminopropyl, ThermoFisher, UK).

TABLE 31
Fatty acid composition of polar lipids purified from egg yolk and krill oil capsules.

| Polar lipid | | | | | | | | | C18:1 | C18:2 |
|---|---|---|---|---|---|---|---|---|---|---|
| fractions | C12:0 | C14:0 | C15:0 | C15:1 | C16:0 | C16:1 | C18:0 | C18:1 | Δ11 | (LA) |
| Egg yolk 1^{st} | 0.0 | 0.6 | 0.1 | 0.2 | 24.7 | 2.2 | 15.6 | 26.9 | 0.0 | 14.8 |
| ppt | | | | | | | | | | |
| Egg yolk 2^{nd} | 0.0 | 0.2 | 0.1 | 0.2 | 27.1 | 1.0 | 16.5 | 23.6 | 0.0 | 20.2 |
| ppt | | | | | | | | | | |
| Krill oil 1^{st} | 0.0 | 3.1 | 0.3 | 0.1 | 25.7 | 1.9 | 1.1 | 6.1 | 0.0 | 2.6 |
| ppt | | | | | | | | | | |
| Krill oil 2^{nd} | 0.0 | 4.0 | 0.4 | 0.1 | ? | 0.2 | 1.1 | 8.3 | 0.1 | 3.0 |
| ppt | | | | | | | | | | |
| | | | | | | C20:4A | C20:5 | | C22:6 | |
| | C18:3 | C19:0 | C20:0 | C20:2 | C20:3 | RA | EPA | C22:2 | DHA | C24:0 |
| Egg yolk 1^{st} | 0.5 | 0.2 | 0.0 | 0.3 | 0.3 | 5.3 | 2.3 | 0.0 | 5.3 | 0.4 |
| ppt | | | | | | | | | | |
| Egg yolk 2^{nd} | 0.4 | 0.0 | 0.0 | 0.4 | 0.3 | 5.3 | ? | 0.0 | 4.2 | 0.1 |
| ppt | | | | | | | | | | |
| Krill oil 1^{st} | 1.9 | 0.0 | 0.0 | 0.2 | 0.2 | 0.8 | 34.7 | 1.3 | 19.0 | 0.6 |
| ppt | | | | | | | | | | |
| Krill oil 2^{nd} | 2.6 | 0.0 | 0.0 | 0.2 | 0.2 | 1.1 | 48.1 | 1.7 | 25.7 | 0.9 |
| ppt | | | | | | | | | | |

The present application claims priority from AU2021901766, filed 11 Jun. 2021 and AU2021903160 filed 1 Oct. 2021, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Adeyo et al. (2011) J Cell Biology 192:1043-1055.
Al-Feel et al. (1992) Proc. Nat1. Acad. Sci. USA. 89:4534-4538.
Arroyo-Caro et al. (2015) J. Agric. Food Chem. 64:277-285.
Athenstaedt and Daum (1997) J. Bacteriol. 179:7611-7616.
Athenstaedt et al. (1999) J. Bacteriol. 181:1458-1463.
Bankaitis et al. (2007). Advances Enzyme Regulation 47:27.
Bhattacharjee et al. (2011) Phytochem 72:166-177.
Becker et al. (1991) Methods in Enzymology 194: 182-7.
Beopoulos et al. (2008) App Env Micro. 74:7779-7789.
Belitz et al., (2009). Food chemistry (4th revised edition). Springer-Verlag, Berlin, Germany.
Bibikova et al. (2002) Genetics 161:1169-1175.
Bligh and Dyer (1959) Can. J. Biochem. Physiol. 37:911-918.
Boeke et al. (1984) Mol. Gen. Genet 197: 345-47.
Calvey et al. (2014) Curr Genet 60:223-230.
Carman and Gil-Soo (2011) Ann Rev Biochem 80:859-883.
Carman and Henry (1989) Annu. Rev. Biochem. 58:635-669.
Carman and Henry (1999) Prog. Lipid Res. 38:361-399.
Carman and Zeimetz (1996) J. Biol. Chem. 271:13293-13296.
Carsanba et al. (2020) Microorganisms 8:1054.
Carter and Kennedy (1966). J. Lipid Res. 7:678-683.
Casaregola et al. (2000) FEBS Lett. 487:95-100.
Celinska et al. (2017) Microb Biotechnol 10: 450-455.
Certo et al. (2012) Nat Methods 8:941-943.
Chang et al. (1998a) J. Biol. Chem. 273:9829-9836.
Chang et al. (1998b) J. Biol. Chem. 273:14933-14941.
Chen et al. (1997) Appl Microbiol Biotechnol. 48:232-5.
Chen et al. (2011) Plant Physiol 155:851-865.
Chisti and Moo-Young (1986). Enzyme and Microbial Technology 8:194-204.
Clancey et al. (1993) J Biol Chem, 268:24580-24590.
Cronan and Vagelos (1972) Biochim Biophys Acta. 265:25-60.
Dabirian et al. (2019) ACS Synthetic Biology 8:1788-1800.
Dani et al. (2011) Plant Biology 13:453-461.
Darvishi et al. (2011) New Biotechnol. 28:756-760.
Dean-Johnson and Henry (1989) J. Biol. Chem. 264:1274-1283.
Delhaize et al. (1999) J Biol Chem 274:7082-7088.
De Castro and Priego-Capote (2010) J Chromatog A 1217:2383-2389.
De Siervo (1969) J Bacteriology 100:1342-1349.
De Virgilio et al. (1992) Yeast 8:1043-1051.
Doyon et al. (2010) Nat. Methods 7:459-460.
Dulermo et al. (2015) Biochim Biophys Acta—Molecular and Cell Biology of Lipids 1851:1107-1117.
Dzugasovi et al. (1998) Current Genetics 34:297-302.
Fakas et al. (2011). J Biol Chem, 286:1464-1474.
Fakas (2017) Eng Life Sci 17:292-302.
Feng et al. (2015) Metabolic Engineering 27:10-19.
Fernandez et al. (1986) J Bacteriol 166:779-786.
Ferreira et al. (2018) Proc. Nat1. Acad. Sci. USA. 115:1262-1267.
Fickers et al. (2003) J Microbiol Methods 55:727-737.
Fischl et al. (1986) J Biol Chem, 261:3178-3183.
Fyrst et al. (1999) Biochemistry 38:5864-5871.
Gasmi et al. (2011) Appl Microbiol Biotechnol 89:109-119.
Ghosal et al. (2007) 1771:1457-1463.
Ghosh et al. (2007) Plant Physiol Biochem 45:887-897.
Gietz et al. (1992) Nuc Acids Res. 27: 69-74.
Gladkowski et al (2011). Food chemistry 126:1013-1018.
Gladkowski et al (2012). J Am Oil Chem Soc 89:179-182.
Greenberg and Lopes (1996) Microbiol. Rev. 60:1-20.
Groenen et al. (1993) Mol. Microbiol. 10:1057-1065.
Gsell et al. (2013) PloS One 8:e77380.
Haft et al. (2005) Computational Biology 1(6):e60.
Hahn-Hagerdal et al. (2005) Microbial Cell Factories 4:31 doi:10.1186/1475-2859-4-31.
Han et al. (2006) J. Biol. Chem. 281:9210-9218.
Han et al. (2008a) J. Biol. Chem. 283:20433-20442.
Han et al. (2008b) J. Biol. Chem. 283:20443-20453.
Han and Carman (2017) J Biol Chem 292:13230-13242.
Harayama (1998) Trends Biotechnol. 16: 76-82.
Hashimoto et al. (2005) J. Biol. Chem. 280:8660-8667.
Hawkins and Kridl (1998) Plant J. 13:743-752.
He et al. (2018) Biotechnol Biofuels 11:258.
Hegel et al (2011) J Supercrit Fluids. 58:68-78.
Hein and Hayen (2012) Metabolites 2:254-267.
Helliwell et al. (1998) Genetics 148:99-112.
Hiesinger et al. (1997) FEBS Letters 415:16-20.
Hildebrand and Law (1964) Biochem. 3:1304-1308.
Hjelmstad and Bell (1988). J. Biol. Chem. 263:19748-19757.
Hjelmstad and Bell (1991). J. Biol. Chem. 266:5094-5103.
Hoe et al. (1999) Emerg. Infect. Dis. 5:254-263.
Holic et al. (2012) Appl Microbiol Biotech. doi:10.1007/s00253-012-3959-6.
Hosaka et al. (1989) J. Biol. Chem. 264:2053-2059.
Ishino et al. (1987) J. Bacteriol. 169:5429-5433.
Ito et al. (1983) J. Bacteriol. 153: 163-8.
Itoh and Kaneko (1974). Yukagaku 23:350-354.
JanBen and Steinbichel (2014) Biotechnology Biofuels 7:1-26.
Jani and Lopes (2009) FEMS Yeast Research 9:552-564.
Janssen et al. (2002) OMICS J. Integ. Biol. 6:23-33.
Jansen et al. (2002) Mol Microbiol 43:1565-1575.
Jones et al. (1995). Plant Cell 7:359-371.
Kamisaka et al. (2015) Appl Microbiol Biotechnol 99:201-210.
Kanemasa et al. (1967) Biochim Biophys Acta 144:382-390.
Kanfer and Kennedy (1964) J. Biol. Chem. 239:1720-1726.
Kassab et al. (2019) Microbial Cell Factories 18:1-10.
Kooistra et al. (2004) Yeast 21: 781-792.
Kim et al. (1996) Proc. Nat1. Acad. Sci. USA 93:1156-1160.
Kim et al. (1999) J. Biol. Chem. 274:14857-14866.
Kim et al. (2012) Genome Res. 22:1327-1333.
Klein et al. (2016) Molecular Biology Cell 27:2014-2024.
Klig and Henry (1984) Proc. Nat1. Acad. Sci. USA 81:3816-3820.
Kodaki and Yamashita (1987) J. Biol. Chem. 262:15428-15435.
Lardizabal et al., (2001). J Biol Chem 276:38862-38869.
Larroude et al. (2018) Biotechnol Adv. 36:2150-2164.
Lee et al. (1994) J Biol Chem 269:19725-19730.
Letts et al. (1983) Proc. Nat1. Acad. Sci. USA 80:7279-7283.
Li et al., (2007) Biochim Biophys Acta—Molecular and Cell Biology of Lipids 1771:1246-1253.
Lõoke et al. (2011) Biotechniques 50: 325-328.
Luke et al. (1996). Molecular Cellular Biology 16:2744-2755.
Lung and Weselake (2006). Lipids 41:1073-1088.
Mauersberger et al. (2001) J Bacteriol DOI: 10.1128/JB.183.17.5102-5109.2001.
Marr and Ingraham (1962). J. Bacteriol 84:1260-1267.
Martins-Noguerol et al. (2020) Heliyon doi.org/10.1016/j.heliyon.2020.e05237.
Masepohl et al. (1996) Biochim. Biophys. Acta 1307:26-30.
McConnell Smith et al. (2009) PNAS 106:5099-5104.
McGraw and Henry (1989). Genetics 122:317-330.
Michaelson et al. (1998) J Biol Chem 273:19055-19059.
Min-Seok et al. (1996) J. Biochem. 120:1040-1047.
Mojica et al. (1995) Mol. Microbiol. 17:85-93.
Mojica et al. (2000) Mol. Microbiol. 36:244-246.
Muller et al. (1998). Yeast 14:1267-1283.
Murray and Greenberg (2000). Mol. Microbiol. 36:651-661.
Nakata et al. (1989) J. Bacteriol. 171:3553-3556.
Napier et al. (1997) Biochem J 328:717-718.
Needleman and Wunsch (1970) J. Mol. Biol. 48:443-453.
Nikawa and Yamashita (1984) Eur. J. Biochem. 143:251-256.
Oh et al. (1997) J Biol Chem 272:17376-17384.
Oldham et al. (2001) Chem. Senses 26:529-531.
Orr-Weaver et al. (1981) PNAS USA 78: 6354-58.
Osman et al. (2010) EMBO J. 29:1976-1987.
Palacios and Wang (2005). J Am Oil Chem Soc 82:571-578.
Pascual et al. (2013) J Biol Chem, 288:35781-35792.
Patel et al. (2018) Molecules 23:1562.
Peter et al. (2017) PLOS ONE 12:e0173739.
Ramirez et al. (2012) Nucleic Acids Res. 40:5560-5568.
Ratledge and Evans (1989) Lipids and their metabolism. In: Rose and Harrison (eds), The yeasts, 2nd edn. Academic, London, pp 367-455.
Ren et al. (2014) Molecular Biology Cell 25:712-727.
Saha et al. (2006) Plant Physiol. 141:1533-1543.
Salas and Ohlrogge (2002). Arch Biochem Biophys 403:25-34.
Schüller et al. (1992) EMBO Journal 11:107-114.
Schneiter et al. (2000) J Bacteriol 182:3655-3660.
Shen et al. (1996) J Biol Chem, 271:789-795.
Shen and Dowhan (1997) J Biol Chem, 272:11215-11220.
Sreenivas et al. (1998) J Biol Chem, 273:16635-16638.
Sreenivas et al. (2001). J Biol Chem, 276:29915-29923.
Sreenivas and Carman (2003) J Biol Chem, 278:20673-20680.
Stukey et al. (1989). J Biol Chem 264:16537-16544.
Summers et al. (1988) Genetics 120:909-922.
Sun et al. (2019) Food Science Nutr. 7:700-710.
Talbot (2011) ‘Sources of saturated and other dietary fats’ in Reducing Saturated Fats in Foods ed Talbot G. Woodhead Publishing, Cambridge.
Tamura et al. (2013). Cell Metabolism 17:709-718.
Tani and Kuge (2014) Yeast 31:145-158.
Tezaki et al. (2017) Biochem Biophys Res Commun. 488:165-170.
Tsukagoshi et al. (1987) Eur. J. Biochem. 169:477-486.
Tripathi et al. (2019) J Biol Chem, 294:19081-19098.
Trotter et al. (1993). J. Biol. Chem. 268:21416-21424.
Trotter and Voelker (1995) J Biol Chem 270:6062-6070.
Turchetto-Zolet et al. (2011) BMC Evolutionary Biology 11:263.
Turchetto-Zolet et al. (2016) Genetics and Molecular Biology 39:524-538. van Embden et al. (2000) J. Bacteriol. 182:2393-2401.
Wang et al. (2012) Genome Res. 22:1316-1326.
Wang et al. (2013) Planta 238:907-922.
Wei et al. (2017) AMB Expr 7:34.
Williams et al. (2008). Biochem. Biophys. Res. Commun. 374: 620-624.
Wongwathanarat et al. (1999). Microbiology 145:2939-2946.
Yen et al. (2002) PNAS USA 99:8512-8517.
Yen et al. (2005) J Lipid Res 46:1502-1511.
Zhang et al. (2014) Cell Mol Life Sci 71:3767-3778.
Zheng and Zou (2001). J. Biol. Chem. 276:41710-16.
Zhou et al. (2008) Insect Mol. Biol. 17:667-676.
Zhou et al. (2013) FEBS Letters 587:2371-2376.
Zienkiewicz et al. (2017) Biotechnology for Biofuels 10 doi:http://dx.doi.org/10.1186/s13068-016-0686-8.
Zulu et al. (2017) Biotechnology for Biofuels, 10 doi:https://doi.org/10.1186/s13068-017-0874-1.

1. Extracted microbial lipid comprising a total fatty acid content which comprises a total saturated fatty acid content of saturated fatty acids (SFA) and a total monounsaturated fatty acid content of monounsaturated fatty acids (MUFA), wherein at least some of the total fatty acid content including at least some of the SFA and at least some of the MUFA is esterified in the form of triacylglycerols (TAG) such that the extracted microbial lipid has a total TAG content, and wherein
(i) the total SFA content of the extracted microbial lipid comprises stearic acid (C18:0), palmitic acid (C16:0), myristic acid (C14:0), arachidic acid (C20:0), behenic acid (C22:0) and lignoceric acid (C24:0), whereby at least 50% by weight of the fatty acids in the total fatty acid content of the extracted microbial lipid are SFA;
(ii) between 20% and 85% by weight of the total fatty acid content is stearic acid;
(iii) the total MUFA content of the extracted microbial lipid comprises oleic acid (C18:1Δ9) and palmitoleic acid (C16:1Δ9), and optionally C16:1Δ7 and/or C17:1;
(iv) the total fatty acid (TFA) content of extracted microbial lipid either lacks polyunsaturated fatty acids (PUFA) or comprises a PUFA content which comprises linoleic acid (C18:2Δ9,12), wherein the PUFA content is less than 5% by weight of the total fatty acid content;
(v) the extracted microbial lipid either comprises polar lipid comprising phospholipid, or lacks polar lipid,
(vi) the extracted microbial lipid is a solid at 25° C., and
(vii) the extracted microbial lipid was obtained from microbial cells, wherein the microbial cells are yeast cells.

2. Extracted microbial lipid comprising a total fatty acid content which comprises a total saturated fatty acid content of saturated fatty acids (SFA) and a total monounsaturated fatty acid content of monounsaturated fatty acids (MUFA), wherein at least some of the total fatty acid content including at least some of the SFA and at least some of the MUFA is esterified in the form of triacylglycerols (TAG) such that the extracted microbial lipid has a total TAG content, and wherein
(i) the total SFA content of the extracted microbial lipid comprises stearic acid (C18:0), palmitic acid (C16:0), myristic acid (C14:0), arachidic acid (C20:0), behenic acid (C22:0) and lignoceric acid (C24:0), whereby at least 50% by weight of the fatty acids in the total fatty acid content of the extracted microbial lipid are SFA;
(ii) between 20% and 85% by weight of the total fatty acid content is stearic acid;
(iii) the total MUFA content of the extracted microbial lipid comprises oleic acid (C18:1Δ9) and palmitoleic acid (C16:1Δ9), and optionally C16:1Δ7 and/or C17:1;
(iv) the total fatty acid (TFA) content of extracted microbial lipid either lacks polyunsaturated fatty acids (PUFA) or comprises a PUFA content which comprises linoleic acid (C18:2Δ9,12), wherein the PUFA content is less than 5% by weight of the total fatty acid content;
(v) the extracted microbial lipid either comprises polar lipid comprising phospholipid, or lacks polar lipid, and
(vi) the extracted microbial lipid is a solid at 25° C.

3. The extracted microbial lipid of claim 1 or 2, wherein the TFA content of the lipid or of the TAG content, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, linoleic acid (LA) by weight, or LA is essentially absent from the TFA of the lipid and/or of the TAG content.

4. The extracted microbial lipid according to any one of claims 1 to 3, wherein the TFA content of the lipid or of the TAG content, or both, comprises less than 4%, less than 3%, less than 2%, less than 1%, less than 0.5% or less than 0.2%, or between 0.2% and 5%, between 0.2% and 4%, between 0.2% and 3%, between 0.2% and 2%, between 0.5% and 5%, between 0.5% and 4%, between 0.5% and 3%, between 0.5% and 2%, PUFA by weight, or PUFA are essentially absent from the TFA content of the lipid and/or the TAG content.

5. The extracted microbial lipid according to any one of claims 1 to 4, wherein the TFA content of the lipid, and/or the TFA content of the TAG, comprises at least 10%, at least 20%, at least 30%, between 10% and 50%, between 10% and 40%, between 10% and 35%, between 10% and 30%, or between 20% and 35%, by weight of oleic acid.

6. The extracted microbial lipid according to any one of claims 1 to 5 which was extracted from a microbe comprising at least one genetic modification and which has a greater amount of one or more or all of total SFA content, C18:0 content, C20:0 content and C22:0 content in the TFA content and/or the TAG content of the lipid compared to a corresponding extracted microbial lipid obtained from a corresponding microbe lacking the at least one genetic modification.

7. The extracted microbial lipid according to any one of claims 1 to 6, wherein the TFA content and/or the TAG content of the lipid comprises between 30% and 90%, between 30% and 80%, between 30% and 70%, between 30% and 60% or between 30% and 50% by weight of SFA.

8. The extracted microbial lipid of claim 7, wherein if the total SFA content is at least 60% by weight of the TFA content of the lipid and/or the TFA content of the TAG, the stearic acid content is at least 40% by weight.

9. The extracted microbial lipid according to any one of claims 1 to 8, wherein the ratio of total saturated fatty acids comprising 18 carbons or more to total saturated fatty acids comprising 16 carbons or less (L/S-SFA ratio) of the TFA content of the lipid or the TAG content of the lipid, or both, is least about 1.5, is least about 2, is least about 2.5, is least about 3, is least about 4, is least about 5, is least about 6, is least about 7, is least about 8, is least about 9, is least about 10, or between about 3 and about 10.

10. The extracted microbial lipid according to any one of claims 1 to 9, wherein the lipid was extracted from a microbe comprising at least one genetic modification and the L/S-SFA ratio of the TFA content or the TAG content of the lipid, or both, is least about 1.5, is least about 2, is least about 2.5, is least about 3, is least about 4, is least about 5, is least about 6, is least about 7, is least about 8, is least about 9, is least about 10, or between about 3 and about 10, fold greater compared to a corresponding extracted microbial lipid obtained from a corresponding microbe lacking the at least one genetic modification.

11. The extracted microbial lipid according to any one of claims 1 to 10, wherein the content of C20:0 fatty acid is at least about 1%, at least about 1.5%, at least about 2%, at least about 2.5% or at least about 3%; the content of C22:0 fatty acid is at least about 1%, at least about 1.5%, at least about 2%, at least about 2.5%, at least about 3% or at least about 3.5%; and/or the content of C24:0 fatty acid is at least about 1%, at least about 1.5%, at least about 2%, at least about 2.5%, at least about 3%, at least about 3.5% or at least 4% by weight of the TFA content of the extracted lipid or the TAG content of the extracted lipid, or both.

12. The extracted microbial lipid according to any one of claims 1 to 11, wherein C20:0, C22:0 and C24:0 fatty acids comprise at least 95%, at least 97% or at least 99%, by weight of the fatty acids of the TFA content of the extracted lipid or the TFA content of the TAG of the lipid, or both, which are at least 20 carbons or longer.

13. The extracted microbial lipid according to any one of claims 1 to 12 which comprises polar lipid, wherein the TFA content of the polar lipid has one or more features as defined in claims 2 to 11 for the TFA content of the extracted lipid or the TAG content.

14. The extracted microbial lipid according to any one of claims 1 to 13, wherein TAG comprises at least 50%, at least 60%, at least 70%, at least 80%, at least 90%, at least 95%, at least 96%, at least 97%, at least 98%, at least 99% or 100% by weight of the total lipid content of the extracted lipid.

15. The extracted microbial lipid according to any one of claims 1 to 14 which comprises a sterol and/or a sterol ester, preferably ergosterol and/or ergosterol ester.

16. The extracted microbial lipid according to any one of claims 1 to 15 which has a moisture content of less than 20% by weight.

17. The extracted microbial lipid according to any one of claims 1 to 16 which has volatile solvent content of less than 10% by weight.

18. The extracted microbial lipid according to any one of claims 1 to 17, wherein the lipid comprises TAG molecules which comprise a MUFA, preferably oleic acid, esterified at their sn-2 position, wherein the ratio of the number of TAG molecules which comprise a MUFA esterified at the sn-2 position to the number of TAG molecules which comprise a fatty acid other than a MUFA esterified at their sn-2 position (MUFA:other FA ratio at sn-2) in the extracted lipid is less than about 0.50, less than about 0.30, less than about 0.20, less than about 0.10, less than about 0.05, less than about 0.04, less than about 0.03 or less than about 0.02.

19. The extracted microbial lipid according to any one of claims 1 to 18 which was extracted from a microbe comprising at least one genetic modification and which comprises polar lipid which comprises phospholipids, wherein at least two, preferably three or all four, of phosphatidylcholine (PC), phosphatidylethanolamine (PE), phosphatidylinositol (PI) and phosphatidylserine (PS) in the extracted lipid have a greater amount of SFA than a corresponding extracted lipid obtained from a corresponding microbe lacking the at least one genetic modification.

20. The extracted microbial lipid according to any one of claims 1 to 19, wherein the lipid was extracted from a microbe comprising genetic modifications including:
(a) an exogenous polynucleotide encoding a FATA fatty acyl-thioesterase,
(b) at least one exogenous polynucleotide encoding at least one fatty acid acyltransferase, preferably at least a diacylglycerol acyltransferase (DGAT), and
(c) a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase, most preferably a null mutation of a FAD2 gene,
wherein each polynucleotide is operably linked to one or more promoters that are capable of directing expression of said polynucleotides in the microbial cells.

21. The extracted microbial lipid of claim 20, wherein the at least a DGAT comprises nucleotides having a sequence as set forth in any one of SEQ ID NOs: 144 to 154, or a nucleotide sequence which is at least 70% identical, preferably at least 80% identical, more preferably at least 90%, at least 95% or at least 97% identical, to any one or more of SEQ ID NOs: 144 to 154 and/or wherein the FATA fatty acyl-thioesterase comprises nucleotides having a sequence as set forth in any one of SEQ ID NOs: 84 or 86, or a nucleotide sequence which is at least 70% identical, preferably at least 80% identical, more preferably at least 90%, at least 95% or at least 97% identical, to any one or more of SEQ ID NOs: 84 or 86.

22. The extracted microbial lipid of claim 20 or 21, wherein the microbe further comprises:
an exogenous polynucleotide encoding an acyl-CoA synthetase (ACS), optionally wherein the ACS comprises nucleotides having a sequence as set forth as any one of SEQ ID NOs: 88 to 89, or a nucleotide sequence which is at least 70% identical, preferably at least 80% identical, more preferably at least 90%, at least 95% or at least 97% identical, to any one or more of SEQ ID NOs: 88 to 89; or
an exogenous polynucleotide encoding a lysophosphatidic acid acyltransferase (LPAAT), optionally wherein the LPAAT comprises nucleotides having a sequence as set forth as any one of SEQ ID NO: 91, or a nucleotide sequence which is at least 70% identical, preferably at least 80% identical, more preferably at least 90%, at least 95% or at least 97% identical, to any one or more of SEQ ID NO: 91.

23. The extracted microbial lipid according to any one of claims 2 to 22 obtained from microbial cells which comprise or consist of eukaryotic cells, fungal cells, bacterial cells or algal cells, living microbial cells, dead microbial cells, or any mixture thereof.

24. The extracted microbial lipid of claim 23, wherein the microbial cells are one or more or all of (i) suitable for fermentation, (ii) oleaginous cells, (iii) non-oleaginous cells, preferably non-oleaginous cells derived from oleaginous cells by genetic modification, and (iv) heterotrophic cells.

25. The extracted microbial lipid of claim 23 or claim 24, wherein the microbial cells are yeast cells.

26. The extracted microbial lipid of claim 2 or 25, wherein the yeast cells are selected from the group consisting of Saccharomyces cerevisiae, Yarrowia hpolytica, Pichia pastoris and any mixture thereof.

27. The extracted microbial lipid of claim 26, wherein the yeast cells are Yarrowia hpolytica.

28. The extracted microbial lipid according to any one of claims 1 to 27 which has a weight of at least 5 g, at least 10 g, at least 50 g or at least 100 g.

29. The extracted microbial lipid according to any one of claims 1 to 28 obtained from microbial cells that have been cultured in the presence of less than 5 g/l, less than 2 g/l, less than 1 g/l of stearate, or have been cultured in the absence of stearate added to the culture medium.

30. Microbial cells comprising lipid as defined in any one of claims 1 to 29.

31. Microbial cells having at least one genetic modification, wherein the microbial cells have (i) an increased saturated fatty acid (SFA) content, an increased content of SFA having at least 18 carbons, an increased content of stearate, an increased content of C20:0 and C22:0 fatty acids, an increased content of C24:0 fatty acid, an increased L/S-SFA ratio, or any combination thereof, in the total fatty acid (TFA) content of the microbial cells or the TFA content of TAG in the microbial cells, or both, and (ii) increased triacylglycerol (TAG) production or accumulation, or both, in each case when compared to corresponding microbial cells lacking the at least one genetic modification and cultured under the same conditions.

32. The microbial cells of claim 31, wherein the TFA content of the cells or the TAG content of the cells, or both, has one or more features as defined in claims 3 to 12 for the TFA content of the extracted lipid or the TAG content.

33. The microbial cells according to any one of claims 30 to 32, wherein the TFA content of the cells, the TAG content of the cells or the polar lipid of the cells, or any combination thereof, has an increased L/S-SFA ratio when compared to the corresponding microbial cells lacking the at least one genetic modification and cultured under the same conditions, preferably wherein the L/S-SFA ratio is least about 1.5, is least about 2, is least about 2.5, is least about is least about 3, is least about 4, is least about 5, is least about 6, is least about 7, is least about 8, is least about 9, is least about 10, or between about 3 and about 10, higher than in the TFA content, the TAG content or the polar lipid of the corresponding microbial cells lacking the at least one genetic modification and cultured under the same conditions.

34. The microbial cells according to any one of claims 30 to 33 which comprise at least one genetic modification which is an exogenous polynucleotide(s) encoding:
a) a FATA fatty acyl-thioesterase,
b) at least one fatty acid acyltransferase, preferably at least a diacylglycerol acyltransferase (DGAT), or
c) a FATA and at least one fatty acid acyltransferase, preferably at least a DGAT,
wherein each polynucleotide is operably linked to one or more promoters that are capable of directing expression of said polynucleotides in the microbial cells.

35. The microbial cells of claim 34 which further comprise an exogenous polynucleotide encoding a lysophosphatidic acid acyltransferase (LPAAT), wherein the polynucleotide is operably linked to one or more promoters that are capable of directing expression of said polynucleotides in the microbial cells.

36. The microbial cells of claim 34 or claim 35, wherein the at least one acyltransferase, preferably at least the DGAT, has at least equal or greater activity on a steroyl-CoA molecule as a substrate compared to palmitoyl-CoA.

37. The microbial cells according to any one of claims 34 to 36, wherein the DGAT comprises amino acids having a sequence set forth as SEQ ID NO: 81, or an amino acid sequence which is at least 60%, at least 70%, at least 80%, at least 90% or at least 95% identical to SEQ ID NO: 81.

38. The microbial cells according to any one of claims 34 to 37, wherein the DGAT is a yeast DGA1.

39. The microbial cells of claim 38, wherein the DGA1 comprises amino acids having a sequence set forth as SEQ ID NO: 53, or an amino acid sequence which is at least 60%, at least 70%, at least 80%, at least 90% or at least 95% identical to SEQ ID NO: 53.

40. The microbial cells according to any one of claims 34 to 39, wherein the FATA comprises amino acids having a sequence set forth as SEQ ID NO: 83 or SEQ ID NO: 85, or an amino acid sequence which is at least 60%, at least 70%, at least 80%, at least 90% or at least 95% identical to one or both of SEQ ID NO: 83 and SEQ ID NO: 85.

41. The microbial cells according to any one of claims 30 to 40 which comprise a genetic modification resulting in a reduction in endogenous Δ12 desaturase expression and/or activity, preferably a genetic modification of an endogenous gene encoding the Δ12 desaturase, more preferably a null mutation of an endogenous gene encoding the Δ12 desaturase.

42. The microbial cells of claim 41, wherein the genetic modification is a mutation in a gene encoding the endogenous Δ12 desaturase, preferably a null mutation of a FAD2 gene.

43. The microbial cells according to any one of claims 30 to 42 which comprise a genetic modification resulting in a reduction in gene expression or activity, or both, of an endogenous gene encoding a DGAT that has a preference for a PUFA-CoA as substrate compared to stearoyl-CoA, or a preference for palmitoyl-CoA compared to stearoyl-CoA, or both, preferably a DGA2 gene.

44. The microbial cells according to any one of claims 30 to 43 which have
1) an exogenous polynucleotide(s) encoding a FATA fatty acyl-thioesterase and a genetic modification resulting in a reduction in endogenous Δ12 desaturase gene expression and/or activity,
2) an exogenous polynucleotide(s) encoding a fatty acid acyltransferase, preferably a diacylglycerol acyltransferase (DGAT), and a genetic modification resulting in a reduction in endogenous Δ12 desaturase gene expression and/or activity,
3) exogenous polynucleotides encoding a FATA fatty acyl-thioesterase and a fatty acid acyltransferase, preferably a diacylglycerol acyltransferase (DGAT), and a genetic modification resulting in a reduction in endogenous Δ12 desaturase gene expression and/or activity,
4) an exogenous polynucleotide(s) encoding a FATA fatty acyl-thioesterase and a genetic modification resulting in a reduction in endogenous DGA2 gene expression and/or activity,
5) an exogenous polynucleotide(s) encoding a fatty acid acyltransferase, preferably a diacylglycerol acyltransferase (DGAT), and a genetic modification resulting in a reduction in endogenous DGA2 gene expression and/or activity,
6) exogenous polynucleotides encoding a FATA fatty acyl-thioesterase and a fatty acid acyltransferase, preferably a diacylglycerol acyltransferase (DGAT), and a genetic modification resulting in a reduction in endogenous DGA2 gene expression and/or activity,
7) an exogenous polynucleotide(s) encoding a FATA fatty acyl-thioesterase, a genetic modification resulting in a reduction in endogenous Δ12 desaturase gene expression and/or activity and a genetic modification resulting in a reduction in endogenous DGA2 gene expression and/or activity,
8) an exogenous polynucleotide(s) encoding a fatty acid acyltransferase, preferably a diacylglycerol acyltransferase (DGAT), a genetic modification resulting in a reduction in endogenous Δ12 desaturase gene expression and/or activity, and a genetic modification resulting in a reduction in endogenous DGA2 gene expression and/or activity, or
9) exogenous polynucleotides encoding a FATA fatty acyl-thioesterase and a fatty acid acyltransferase, preferably a diacylglycerol acyltransferase (DGAT), a genetic modification resulting in a reduction in endogenous Δ12 desaturase gene expression and/or activity, and a genetic modification resulting in a reduction in endogenous DGA2 gene expression and/or activity,
wherein each polynucleotide is operably linked to one or more promoters that are capable of directing expression of said polynucleotides in the microbial cells.

45. The microbial cells according to any one of claims 30 to 44 which comprises exogenous polynucleotides encoding a FATA fatty acyl-thioesterase, a fatty acid acyltransferase, preferably a diacylglycerol acyltransferase (DGAT) and a genetic modification resulting in a reduction in endogenous Δ12 desaturase gene expression and/or activity, preferably a mutation in a gene encoding the endogenous Δ12 desaturase, more preferably a null mutation of a FAD2 gene.

46. The microbial cells of claim 45, wherein the DGAT comprises nucleotides having a sequence as set forth as any one of SEQ ID NOs: 144 to 154, or a nucleotide sequence which is at least 70% identical, preferably at least 80% identical, more preferably at least 90%, at least 95% or at least 97% identical, to any one or more of SEQ ID NOs: 144 to 154 and/or the FATA fatty acyl-thioesterase comprises nucleotides having a sequence as set forth as any one of SEQ ID NOs: 84 or 86, or a nucleotide sequence which is at least 70% identical, preferably at least 80% identical, more preferably at least 90%, at least 95% or at least 97% identical, to any one or more of SEQ ID NOs: 84 or 86.

47. The microbial cells of claim 45 or 46, further comprising an exogenous polynucleotide encoding an acyl-CoA synthetase (ACS), optionally wherein the ACS comprises nucleotides having a sequence as set forth as any one of SEQ ID NOs: 88 to 89, or a nucleotide sequence which is at least 70% identical, preferably at least 80% identical, more preferably at least 90%, at least 95% or at least 97% identical, to any one or more of SEQ ID NOs: 88 to 89; or an exogenous polynucleotide encoding a lysophosphatidic acid acyltransferase (LPAAT), optionally wherein the LPAAT comprises nucleotides having a sequence as set forth in SEQ ID NO: 91, or a nucleotide sequence which is at least 70% identical, preferably at least 80% identical, more preferably at least 90%, at least 95% or at least 97% identical, to SEQ ID NO: 91.

48. The microbial cells according to any one of claims 30 to 47 which comprise or consist of eukaryotic cells, fungal cells, bacterial cells or algal cells, living microbial cells, dead microbial cells, or any mixture thereof.

49. The microbial cells according to any one of claims 30 to 48 which are one or more or all of (i) suitable for fermentation, (ii) oleaginous cells, (iii) non-oleaginous cells, preferably non-oleaginous cells derived from oleaginous cells by genetic modification, and (iv) heterotrophic cells.

50. The microbial cells according to any one of claims 30 to 49 which are yeast cells.

51. The microbial cells of claim 50, wherein the yeast cells are selected from the group consisting of Saccharomyces cerevisiae, Yarrowia lipolytica, Pichia pastoris and any mixture thereof.

52. The microbial cells of claim 51, wherein the yeast cells are Yarrowia lipolytica.

53. The microbial cells according to any one of claims 30 to 52 in a culture medium having less than about 5 g/l, less than about 2 g/l, less than about 1 g/l of stearate, or no added stearate, or have been cultured in said medium.

54. A microbial cell extract comprising lipid as defined in any one of claims 1 to 29 or produced from the microbial cells of any one of claims 30 to 53.

55. A DNA construct, or a combination of DNA constructs, which encodes one or more of the enzymes according to any one of claims 34 to 40, preferably integrated into the genome of a microbial cell.

56. A process for producing extracted lipid, comprising
(a) obtaining microbial cells according to any one of claims 30 to 53, and
(b) extracting lipid from the microbial cells,
so as to thereby produce the extracted lipid.

57. The process of claim 56 which further comprises culturing the microbial cells, or a step of treating the cells with an acid prior to step (b).

58. The process of claim 57, wherein cells are cultured in medium having less than 5 g/l, less than 2 g/l, less than 1 g/l of stearate, or no stearate.

59. The process according to any one of claims 56 to 58, wherein the step of extracting the lipid comprises exposing the cells to an organic solvent, pressing the cells or treating the cells with microwave irradiation, ultrasonication, high-speed homogenization, high-pressure homogenization, bead beating, autoclaving, thermolysis, or any combination thereof.

60. The process according to any one of claims 56 to 59, wherein the method further comprises modifying or purifying the lipid after extraction.

61. A process for culturing microbial cells, the process comprising
(a) obtaining microbial cells according to any one of claims 30 to 53, and
(b) increasing the number of the cells by culturing the cells in a suitable medium.

62. A process for producing a microbial cell which produces lipid as defined in any one of claims 1 to 29, the process comprising a step of introducing one or more genetic modifications and/or exogenous polynucleotides as defined in any one of claims 34 to 47 into a progenitor microbial cell.

63. The process of claim 62, which comprises one or more steps of
(i) producing progeny cells from the cell comprising the introduced genetic modifications and/or exogenous polynucleotides,
(ii) mutagenesis of a population of progenitor cells,
(iii) introduction of one or more exogenous polynucleotides whereby the exogenous polynucleotides become integrated into the genome of the microbial cell, preferably into a predetermined location,
(iv) determining the fatty acid composition of the cell or progeny cells thereof, and
(v) selecting a progeny cell which comprises lipid as defined in any one of claims 1 to 29.

64. A composition comprising one or more or all of the lipid according to any one of claims 1 to 29, the microbial cell according to any one of claims 30 to 53, or the microbial cell extract of claim 54.

65. The composition of claim 64 comprising another food, feed or beverage ingredient.

66. The composition of claim 64 or claim 65 which further comprises one or more fatty acids, esterified or non-esterified, from a source other than the extracted microbial lipid, cell or extract.

67. A food, feedstuff or beverage comprising an ingredient which is one or more or all of the lipid according to any one of claims 1 to 29, the microbial cell according to any one of claims 30 to 53, the microbial cell extract of claim 54, or the composition according to any one of claims 64 to 66, and at least one other food, feed or beverage ingredient.

68. The food, feedstuff or beverage of claim 67, which lacks components obtained from an animal.

69. The food, feedstuff or beverage of claim 67 or claim 68, wherein the food, feedstuff or beverage is a meat substitute.

70. A method of producing a food, feedstuff or beverage, the method comprising combining one or more or all of the lipid according to any one of claims 1 to 29, the microbial cell according to any one of claims 30 to 53, the microbial cell extract of claim 54, or the composition according to any one of claims 64 to 66, with at least one other food, feed or beverage ingredient.

71. Use of one or more or all of the lipid according to any one of claims 1 to 29, the microbial cell according to any one of claims 30 to 53, the microbial cell extract of claim 54, or the composition according to any one of claims 64 to 66 to produce a food, feedstuff or beverage ingredient, or a food, feedstuff or beverage.


SEQUENCE LISTING (176 sequences)

SEQ ID NO: 1 - protein, 419 aa, Yarrowia lipolytica
MDSTTQTNTGTGKVAVQPPTAFIKPIEKVSEPVYDTFGNEFTPPDYSIKDILDAIPQECYKRSYVKSYSYVARDCFFIAVFAYMAYAYLPLIPSASGRAVAWAMYSIVQGLFGTGLWVLAHECGHSAFSDSNTVNNVTGWVLHSSMLVPYYAWKLTHSMHHKSTGHLTRDMVFVPKDRKEFMENRGAHDWSELAEDAPLMTLYGLITQQVFGWPLYLLSNVTGQKYPKLNKWAVNHFNPNAPLFEKKDWFNIWISNVGIGITMSVIAYSINRWGLASVTLYYLIPYLWVNHWLVAITYLQHTDPTLPHYHADQWNFTRGAAATIDREFGFIGSFCFHDIIETHVLHHYVSRIPFYNARIATEKIKKVMGKHYRHDDTNFIKSLYTVARTCQFVEGKEGIQMFRNVNGVGVAPDGLPSKK

SEQ ID NO: 2 - DNA, 3260 bp, Yarrowia lipolytica
aactatacaagtatcaagttgcccattgtgttgtatgttccagaaccattgtacccgtacaagtagttaagcatagcccttgtgcaagagtgtgtacgatcaatgtctcagatacggcggtgactgccgcttctggcagtctttcaacgctgcgtttcttcgtcgatggaccggaagagctctctttacgagccaaaggctccaagagtttggttgtcttgtactctctatctttccacaccgccgcccacaaggttcggaacgacatctcccaactctaactttaagcatggcctgttgagaagccatgctattcatcggacacctgttcaacgagtcaacaagcggacatagtcgtcaagtagtggcagcacagagagagagtcgtgccaatgggggtcatgtagtagcacatgacgaaataagagacgttggattcatgtagaaccgacgtagtcacgtgaacacgtccaaggtcttcccctcttcacgtgcacgtgtaactggttctttcactctcccccccgccataagtcgacccatgtcacggttgagacgtggtccctccacactatccacgctgagagcagcagtataccccgggcggttccgttcggagctctcttggccaatccgcacatgcgcattatgcgtttttcaggtggtaacagacgaggagtacgagcgtgttaaagtggcggttaggagaggcggggcccgtagttatatacaagaggtagatgcgtgctggtgttagaggggctctcaggattaggaggaaaatttgacattggccctcaacatataacctcgggtgtgcctctgtttaccctcagcttttgcttgtccccaagtcagtcacgccaggccaaaaaggttggtggattgacagggagaaaaaaaaaagcctagtgggtttaaactcgaggtaagacattgaaatatataccggtcggcatcctgagtccctttctcgtattccaacagaccgaccatagaaatggattcgaccacgcagaccaacaccggcaccggcaaggtggccgtgcagccccccacggccttcattaagcccattgagaaggtgtccgagcccgtctacgacacctttggcaacgagttcactcctccagactactctatcaaggatattctggatgccattccccaggagtgctacaagcggtcctacgttaagtcctactcgtacgtggcccgagactgcttctttatcgccgtttttgcctacatggcctacgcgtacctgcctcttattccctcggcttccggccgagctgtggcctgggccatgtactccattgtccagggtctgtttggcaccggtctgtgggttcttgcccacgagtgtggccactctgctttctccgactctaacaccgtcaacaacgtcaccggatgggttctgcactcctccatgctggtcccttactacgcctggaagctgacccactccatgcaccacaagtccactggtcacctcacccgtgatatggtgtttgtgcccaaggaccgaaaggagtttatggagaaccgaggcgcccatgactggtctgagcttgctgaggacgctcccctcatgaccctctacggcctcatcacccagcaggtgtttggatggcctctgtatctgctgtctaacgttaccggacagaagtaccccaagctcaacaaatgggctgtcaaccacttcaaccccaacgccccgctgtttgagaagaaggactggttcaacatctggatctctaacgtcggtattggtatcaccatgtccgtcatcgcatactccatcaaccgatggggcctggcttccgtcaccctctactacctgatcccctacctgtgggtcaaccactggctcgtggccatcacctacctgcagcacaccgaccccactctgccccactaccacgccgaccagtggaacttcacccgaggagccgccgccaccatcgaccgagagtttggcttcatcggctccttctgcttccatgacatcatcgagacccacgttctgcaccactacgtgtctcgaattcccttctacaacgcccgaatcgccactgagaagatcaagaaggtcatgggcaagcactaccgacacgacgacaccaacttcatcaagtctctttacactgtcgcccgaacctgccagtttgttgaaggtaaggaaggcattcagatgtttagaaacgtcaatggagtcggagttgctcctgacggcctgccttctaaaaagtagagctagaaatgttatttgattgtgttttaactgaacagcaccgagcccgaggctaagccaagcgaagccgaggggttgtgtagtccatggacgtaacgagtaggcgatatcaccgcactcggcactgcgtgtctgcgttcatgggcgaagtcacattacgctgacaaccgttgtagtttccctttagtatcaatactgttacaagtaccggtactcgtactcgtactgatacgaatctgtgggaagaagtcacccttatcagaccttcatactgatgtttcggatatcaatagaactggcatagagccgttaaagaagtttcacttaatcactccaaccctcctacttgtagattcaagcagatcgataagatggatttgatggtcagtgctagcctgggcccgagcgtcaccatcagtcattctttacaagttgttccactcttctccctacttgaatcgatctgtctgggcgcctgatattcgttcgacttgtacgatagcggggtgtatttcgagctcgccgaaaccggttttccatgaatttccaccgataattccattggcaattcaatcgaccataagcgtatccggtaacggtgagacattgagacaccgatcgattagagagactactgtacgagcagtctactgtatattagaaggaacaataggtagtccaactgagcttttaactagtgggtgttttttgaacgtgtttggagatcttattaagcaaaaaaaaagctgtgttcacattaacaccaacaaaccctgccggtagttccttcaaaaacgccacatgcatcaatcatcttcagctttgccgatctcctcctgccgaggttacaaccaactggagccatctttatcgtgccgtagagaagcgcagccagggagagtttgagaccggcaaaacgagggaatcggtagattctcagaaataatccatatggagtcacgtgatcagacttcccatggacttttatattgatatttgt

SEQ ID NO: 3 - DNA, 1620 bp, Artificial Sequence
  Construct encoding hygromycin resistance selectable marker gene
tagggataacagggtaatgataacttcgtatagcatacattatacgaagttatgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcacaatgaagaagcccgagctgaccgctacctctgttgagaagttcctgattgagaagtttgattccgtttccgacctgatgcagctgtccgagggcgaggagtctcgagccttctcctttgacgtgggcggacgaggttacgttctgcgagtgaactcgtgtgccgacggcttctacaaggatcgatacgtctaccgacactttgcttctgccgctctgcccatccctgaggttctcgacattggcgagttctctgagtccctcacctactgcatctctcgacgagctcagggagtcaccctgcaggacctccctgagactgagctgcctgctgtcctccagcctgttgctgaggccatggacgctatcgctgctgctgatctgtcccagacctcgggtttcggcccctttggacctcagggaattggacagtacaccacttggcgagacttcatctgtgctattgccgatcctcacgtctaccattggcagaccgttatggacgatactgtgtcggcttctgtcgctcaggctctggacgagctgatgctctgggccgaggattgccccgaggttcgacacctggtgcatgctgacttcggttccaacaacgttctcaccgacaacggccgaatcactgccgtgattgactggtccgaggctatgtttggcgactcgcagtacgaggtggccaacatcttcttttggcgaccctggctggcttgtatggagcagcagacccgatacttcgagcgacgacatcctgagctcgctggctcccctcgactgcgagcttacatgctccgaattggtctggaccagctctaccagtcgctggtggatggcaactttgacgatgctgcctgggctcagggacgatgtgacgccatcgtgcgatctggcgctggaaccgtcggacgaactcagattgcccgacgatccgctgctgtctggaccgacggatgcgtggaggtcctggctgattcgggtaaccgacgaccctctactcgacctcgagctaaggagtaagtgtctgtggtatctaagctatttatcactctttacaacttctacctcaactatctactttaataaatgaatatcgtttattctctatgattactgtatatgcgttcctctaagacaaatcgaatataacttcgtatagcaggagttatccgaagcgagtagggataacagggtaat

SEQ ID NO: 4 - protein, 341 aa, Escherichia coli
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 5 - DNA, 1165 bp, Artificial Sequence
  Construct encoding nourseothricin resistance selectable marker gene
tagggataacagggtaatgataacttcgtatagcatacattatacgaagttatggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcacaatgaccactctggatgacaccgcttaccgataccgaacttccgttcctggcgatgccgaggctattgaggctctggatggatctttcaccactgacaccgttttccgagtgaccgctactggcgacggcttcaccctgcgagaggtgcctgtcgaccctcctctcaccaaggttttccctgacgatgagtcggacgatgagtctgacgctggagaggacggcgaccctgactctcgaactttcgtggcttacggcgacgatggcgacctggccggctttgtggtcgtttcttactccggatggaaccgacgactgaccgtggaggacatcgaggtcgctcctgagcaccgaggtcatggtgtcggacgagctctgatgggtctggctactgagttcgctcgagagcgaggtgctggccacctgtggctcgaggtcaccaacgttaacgcccctgctattcatgcctaccgacgaatgggttttaccctgtgtggcctcgatactgccctgtacgacggaaccgcttccgatggagagcaggccctctacatgtcgatgccctgcccttaagtgtctgtggtatctaagctatttatcactctttacaacttctacctcaactatctactttaataaatgaatatcgtttattctctatgattactgtatatgcgttcctctaagacaaatcgaatataacttcgtatagcaggagttatccgaagcgagtagggataacagggtaat

SEQ ID NO: 6 - protein, 189 aa, Streptomyces noursei
MTTLDDTAYRYRTSVPGDAEAIEALDGSFTTDTVFRVTATGDGFTLREVPVDPPLTKVFPDDESDDESDAGEDGDPDSRTFVAYGDDGDLAGFVVVSYSGWNRRLTVEDIEVAPEHRGHGVGRALMGLATEFARERGAGHLWLEVTNVNAPAIHAYRRMGFTLCGLDTALYDGTASDGEQALYMSMPCP

SEQ ID NO: 7 - DNA, 417 bp, Yarrowia lipolytica
tagggataacagggtaatgataacttcgtatagcatacattatacgaagttatgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcaca

SEQ ID NO: 8 - DNA, 33 bp, Artificial Sequence
  Nucleotide sequence of artificial oligonucleotide primer at 063 SacII-Hyg/Nos_F
gggccgcggtagggataacagggtaatgataac

SEQ ID NO: 9 - DNA, 32 bp, Artificial Sequence
  Nucleotide sequence of artificial oligonucleotide primer at 064 SacII-Hyg/Nos_R
cccccgcggattaccctgttatccctactcgc

SEQ ID NO: 10 - DNA, 34 bp, Artificial Sequence
  Nucleotide sequence of artificial oligonucleotide primer at 213 LRO_DelCas5F
gggggcgcgccgagtcgactttccgatacagaag

SEQ ID NO: 11 - DNA, 34 bp, Artificial Sequence
  Nucleotide sequence of artificial oligonucleotide primer at 214 LRO_DelCas5R
cccgcgatcgctgatggttgtgatcaaccggaaa

SEQ ID NO: 12 - DNA, 36 bp, Artificial Sequence
  Nucleotide sequence of artificial oligonucleotide primer at 215 LRO_DelCas3F
ggggcgatcgcagagtccgttttgtagagtaatatg

SEQ ID NO: 13 - DNA, 35 bp, Artificial Sequence
  Nucleotide sequence of artificial oligonucleotide primer at 216 LRO_DelCas3R
cccgcggccgcacggacctcgcaagttgcaaaaag

SEQ ID NO: 14 - DNA, 31 bp, Artificial Sequence
  Nucleotide sequence of artificial oligonucleotide primer at 217 DGA1_DelCas5F
gggggcgcgccatgctgcgggcggatcctgg

SEQ ID NO: 15 - DNA, 35 bp, Artificial Sequence
  Nucleotide sequence of artificial oligonucleotide primer at 218 DGA1_DelCas5R
cccgcgatcgcagcttttgttttgtgtgacttgtc

SEQ ID NO: 16 - DNA, 32 bp, Artificial Sequence
  Nucleotide sequence of artificial oligonucleotide primer at 219 DGA1_DelCas3F
ggggcgatcgcggaaaactgcctgggttaggc

SEQ ID NO: 17 - DNA, 32 bp, Artificial Sequence
  Nucleotide sequence of artificial oligonucleotide primer at 220 DGA1_DelCas3R
cccgcggccgctctgatggcctggagcgagtt

SEQ ID NO: 18 - DNA, 35 bp, Artificial Sequence
  Nucleotide sequence of artificial oligonucleotide primer at 221 DGA2_DelCas5F
gggggcgcgcctgggagtgtatttggaaaatgact

SEQ ID NO: 19 - DNA, 31 bp, Artificial Sequence
  Nucleotide sequence of artificial oligonucleotide primer at 222 DGA2_DelCas5R
cccgcgatcgctttgcgggcggtacgggtac

SEQ ID NO: 20 - DNA, 36 bp, Artificial Sequence
  Nucleotide sequence of artificial oligonucleotide primer at 223 DGA2_DelCas3F
ggggcgatcgccataacactcatcagtagcctttac

SEQ ID NO: 21 - DNA, 39 bp, Artificial Sequence
  Nucleotide sequence of artificial oligonucleotide primer at 224 DGA2_DelCas3R
cccgcggccgctcttgtaattccatagataatatatacg

SEQ ID NO: 22 - DNA, 34 bp, Artificial Sequence
  Nucleotide sequence of artificial oligonucleotide primer at 225 ARE1_DelCas5F
gggggcgcgccgccgacaatctctcttcctcatt

SEQ ID NO: 23 - DNA, 33 bp, Artificial Sequence
  Nucleotide sequence of artificial oligonucleotide primer at 226 ARE1_DelCas5R
cccgcgatcgctgtgtgtgcggagagtgtcttg

SEQ ID NO: 24 - DNA, 32 bp, Artificial Sequence
  Nucleotide sequence of artificial oligonucleotide primer at 227 ARE1_DelCas3F
ggggcgatcgcgcacagtcgcttcaccacttg

SEQ ID NO: 25 - DNA, 37 bp, Artificial Sequence
  Nucleotide sequence of artificial oligonucleotide primer at 228 ARE1_DelCas3R
cccgcggccgctaaaacaatgacaaatacaactctag

SEQ ID NO: 26 - DNA, 35 bp, Artificial Sequence
  Nucleotide sequence of artificial oligonucleotide primer at 229 AsiSI-Hyg/Nos_F
ggggcgatcgctagggataacagggtaatgataac

SEQ ID NO: 27 - DNA, 34 bp, Artificial Sequence
  Nucleotide sequence of artificial oligonucleotide primer at 230 AsiSI-Hyg/Nos_R
cccgcgatcgcattaccctgttatccctactcgc

SEQ ID NO: 28 - DNA, 19 bp, Artificial Sequence
  Nucleotide sequence of artificial oligonucleotide primer at 239 Fad2-gene_F
gtgtccgagcccgtctacg

SEQ ID NO: 29 - DNA, 22 bp, Artificial Sequence
  Nucleotide sequence of artificial oligonucleotide primer at 240 Fad2-gene_R
acatggtgataccaataccgac

SEQ ID NO: 30 - DNA, 20 bp, Artificial Sequence
  Nucleotide sequence of artificial oligonucleotide primer at 241 ARE1KOScreen_F1
gatgcaaatgacgcacggcc

SEQ ID NO: 31 - DNA, 20 bp, Artificial Sequence
  Nucleotide sequence of artificial oligonucleotide primer at 242 ARE1KOScreen_F2
ccaaacgacactcgatagtg

SEQ ID NO: 32 - DNA, 20 bp, Artificial Sequence
  Nucleotide sequence of artificial oligonucleotide primer at 243 ARE1KOScreen_R1
tgctttgcgtgggagagctc

SEQ ID NO: 33 - DNA, 24 bp, Artificial Sequence
  Nucleotide sequence of artificial oligonucleotide primer at 244 ARE1KOScreen_R2
cagtggttgacattctataggcac

SEQ ID NO: 34 - DNA, 21 bp, Artificial Sequence
  Nucleotide sequence of artificial oligonucleotide primer at 245 DGA1KOScreen_F1
caggcaacagacaagtcacac

SEQ ID NO: 35 - DNA, 20 bp, Artificial Sequence
  Nucleotide sequence of artificial oligonucleotide primer at 246 DGA1KOScreen_F2
catctggctccaacggccac

SEQ ID NO: 36 - DNA, 20 bp, Artificial Sequence
  Nucleotide sequence of artificial oligonucleotide primer at 247 DGA1KOScreen_R1
cagctgtgggtcgttttcgc

SEQ ID NO: 37 - DNA, 20 bp, Artificial Sequence
  Nucleotide sequence of artificial oligonucleotide primer at 248 DGA1KOScreen_R2
gctatttgcctaacccaggc

SEQ ID NO: 38 - DNA, 21 bp, Artificial Sequence
  Nucleotide sequence of artificial oligonucleotide primer at 249 DGA2KOScreen_F1
catagctacacccaaagtcgc

SEQ ID NO: 39 - DNA, 20 bp, Artificial Sequence
  Nucleotide sequence of artificial oligonucleotide primer at 250 DGA2KOScreen_F2
cgctcagcggactgaccatg

SEQ ID NO: 40 - DNA, 20 bp, Artificial Sequence
  Nucleotide sequence of artificial oligonucleotide primer at 251 DGA2KOScreen_R1
tcttggggtacacgggctgg

SEQ ID NO: 41 - DNA, 22 bp, Artificial Sequence
  Nucleotide sequence of artificial oligonucleotide primer at 252 DGA2KOScreen_R2
ctgtaaaggctactgatgagtg

SEQ ID NO: 42 - DNA, 22 bp, Artificial Sequence
  Nucleotide sequence of artificial oligonucleotide primer at 257 LRO1KOScreen_F1
catgactaatcattcacgcgac

SEQ ID NO: 43 - DNA, 20 bp, Artificial Sequence
  Nucleotide sequence of artificial oligonucleotide primer at 258 LRO1KOScreen_F2
cgtggtcggtgtgatagccg

SEQ ID NO: 44 - DNA, 20 bp, Artificial Sequence
  Nucleotide sequence of artificial oligonucleotide primer at 259 LRO1KOScreen_R1
gttcgcagcagatcggctcg

SEQ ID NO: 45 - DNA, 22 bp, Artificial Sequence
  Nucleotide sequence of artificial oligonucleotide primer at 260 LRO1KOScreen_R2
cgaacgcccttctccatcagtg

SEQ ID NO: 46 - DNA, 20 bp, Artificial Sequence
  Nucleotide sequence of artificial oligonucleotide primer at 270 URA3KOScrnF1
cgttggaggctgtgggtctg

SEQ ID NO: 47 - DNA, 20 bp, Artificial Sequence
  Nucleotide sequence of artificial oligonucleotide primer at 271 URA3KOScrnF2
agtgctcaagctcgtggcag

SEQ ID NO: 48 - DNA, 20 bp, Artificial Sequence
  Nucleotide sequence of artificial oligonucleotide primer at 272 URA3KOScrnR1
gatctcggttctggccgtac

SEQ ID NO: 49 - DNA, 20 bp, Artificial Sequence
  Nucleotide sequence of artificial oligonucleotide primer at 273 URA3KOScrnR2
gcctccaggaagtccatggg

SEQ ID NO: 50 - protein, 286 aa, Yarrowia lipolytica
MPSYEARANVHKSAFAARVLKLVAAKKTNLCASLDVTTTKELIELADKVGPYVCMIKTHIDIIDDFTYAGTVLPLKELALKHGFFLFEDRKFADIGNTVKHQYKNGVYRIAEWSDITNAHGVPGTGIIAGLRAGAEETVSEQKKEDVSDYENSQYKEFLVPSPNEKLARGLLMLAELSCKGSLATGEYSKQTIELARSDPEFVVGFIAQNRPKGDSEDWLILTPGVGLDDKGDALGQQYRTVEDVMSTGTDIIIVGRGLYGQNRDPIEEAKRYQKAGWEAYQKINC

SEQ ID NO: 51 - DNA, 2861 bp, Yarrowia lipolytica
ttaccattttaaagcaattgttaacaagatactactgtagaaaactgattaaacattcgtaacttgcctgtggctcgacttacagccaaatgttcgcggcaaaatttgaatattttgcacacttattgtcccaaaaccaaataatgccaacaaaatccgatatagtgtaggggctatcacataccctctcataagaatacttttaaccgtggagacggggttcgattcgtatcggagtgtttattttttgctcaaccataccctggggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacgaagctctaaagctgaaacgggacatcgaaaagttggagaagtcaatctggaaactacacgtagaaaacagacaccatctccaacaagtacaggacttcaaatttgcactgaagacattctgtgggtggagccataagtaacaatcgcctgctccaggaacttaaaaaagaggcattataggccccttcatctgaatattctttgagtaccaactgccgccaactccccaatcgttaattagtgttatctattctatctatgctattagcacatatttatcatgtgctgctgttatgtacgggttcaatatacaaatgcttagtcctcaaacaccttcataggtgggtcgtcttcctggccaaatcgaattctcatgatttcaaacacgaacagaccgatagacgtagacggaagcgcactgagggtcgtccagccacaaccatggtataggtatctgggccaactcaaccgcagaccctttcggatgttattggctgtatgcagagtctcaacgtaggctcggtagtagagatg

SEQ ID NO: 52 - DNA, 3545 bp, Yarrowia lipolytica
atgctgcgggcggatcctggtgcatttttgcttgcgattgttgtgaaaattagcacggttatattagcccgtaataaatgcccgtctccatcttcatatggccatcaccccgcaaatagccggccaatcaggcgtatgtcacctgttgctcacacgcatgtcctcggaccgttgtattgtgcaagtaggggtacctccccgatccatcctcgaccagtggcacgctcaaccccatggttcgcttttctcttttcgtctatcctgaactgagtttttttccacgccaactgatatccccttacgttaccccctcatcacctggtgaggcgaaactgtaaggtgaaagctaaaaatgacatctcagctgcacgaaggaccggggcttaaaagacgggctggtgcttgtgatttaaaactggacaaatctcagcttgcttgaaattttggtctccaactgtttccgagcgaatcgcacacaaaccgggcttctctctgcagaccacgcccccgaaactctttctcccaccaccaccaacactccctttccattcccacaccgttcctctctcgtgcgtcatccttgcgcaatcatcttcgtctgcgacatattgtacgacatacagtaccacggaacgtttcagaccgtcgacgtgaacacatcttaggaacagcaacctgagctacagaaatctatctataggcggataaaaaaacgcacccactgctcgtcctccttgctcctcgaaaccgactcctctacacacgtcaaatccgaggttgaaatcttccccacatttggcagccaaaccagcacatcccagcaacctcgcacagcgccgaaatcgacctgtcgacttggccacaaaaaaaagcaccggctctgcaacagttctcacgaccaattacgtacaagtacgaaatcgttcgtggaccgtgactgataagctcccactttttcttctaacaacaggcaacagacaagtcacacaaaacaaaagctatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaaggaaaactgcctgggttaggcaaatagctaatgagtatttttttgatggcaaccaaatgtagaaagaaaaaaaaaaaaaaagaaaaaaaaaagagaatattatatctatgtaattctattaaaagctctgttgagtgagcggaataaatactgttgaagaggggattgtgtagagatctgtttactcaatggcaaactcatctgggggagatccttccactgtgggaagctcctggatagcctttgcatcgggttcaagaagaccattgtgaacagccttgacactgtcgacaatgacagcatcaacgccatgcttgatctgggtcttggcaatcacagggttgttattcagaacaccgtaggagacacagaccagcccagcccgcttgacgacctggataagtcgagggcacatgacaaagggactacactcggacacaagacccaaaaggttccaatccttgcagaaccgaatggcctcctggagagaagaggctcgaatgtcatacatgggggcagttccagactcggtgaggaacagggtggggtatccgacagggttgacttcagtcttgccaatcatagggacacctccttgcttgtggttcagcatgaggcacacgtcagggtggaaagaggagaagataatctctcggtttgctccatactcaaagacacactcgaggaccttgtccgcccaaacgttgaggtcaagggcaaactcttccatctcctcagcttgtccttcgtcaagcatggggtacttgcactcgatgttgcagccaacgttgggagggagagactgtagaacctctcgtagagtggtgaaaggggcctgaatggaaataccacgagaattacccttgaaacccttcatcttgtagtcacgagtgtacttcattcgatgagtgatgtggtcatctgcgtcagggtcatcttgctgaagtgcagttccttcaagtatcttgtcgccctcagaaactcgctccaggccatcaga

SEQ ID NO: 53 - protein, 514 aa, Yarrowia lipolytica
MTIDSQYYKSRDKNDTAPKIAGIRYAPLSTPLLNRCETFSLVWHIFSIPTFLTIFMLCCAIPLLWPFVIAYVVYAVKDDSPSNGGVVKRYSPISRNFFIWKLFGRYFPITLHKTVDLEPTHTYYPLDVQEYHLIAERYWPQNKYLRAIISTIEYFLPAFMKRSLSINEQEQPAERDPLLSPVSPSSPGSQPDKWINHDSRYSRGESSGSNGHASGSELNGNGNNGTTNRRPLSSASAGSTASDSTLLNGSLNSYANQIIGENDPQLSPTKLKPTGRKYIFGYHPHGIIGMGAFGGIATEGAGWSKLFPGIPVSLMTLTNNFRVPLYREYLMSLGVASVSKKSCKALLKRNQSICIVVGGAQESLLARPGVMDLVLLKRKGFVRLGMEVGNVALVPIMAFGENDLYDQVSNDKSSKLYRFQQFVKNFLGFTLPLMHARGVFNYDVGLVPYRRPVNIVVGSPIDLPYLPHPTDEEVSEYHDRYIAELQRIYNEHKDEYFIDWTEEGKGAPEFRMIE

SEQ ID NO: 54 - DNA, 3581 bp, Yarrowia lipolytica
tgggagtgtatttggaaaatgacttggcctggaccccgactaccgctagtgctacccatggggtgtctccagaacccttgtacatcagcctatatgcgtgtgtgtctacgtctactgtacaaatctgcttggcgtcaaatggcactacaagtacaaagaccaaatgccacaagttcagtacgagtacaagagtctgctacttgtagctcccgcaggtatttagttcatacaaacgagtatacttgtagctgtccaacagtagttttcagacacctgatcagaaataggaacacgctccacggacctcgtatccactctaagaccataatatatgggtttctggaacattggtgcccctcacctctcatctcttttttttttacctctctgaccaccaccacccttgttgttccgagtggttagctgcaccccacctagcaaactttagggccccatacgcataagcatacgtccggtgtgcgacttgtgtgtgaacgcggtccacacaaggtgtctttcaagtccacacagggctttgtctagagcctgggctatcttcccacgtttgtatatatagactggatccacacccagcaacgcaacgtcctcccacccaaccctatcctccacgttacacgcccaagatcttccaaacaagaagacacattccacagcaagaataaccttggcaagaacgtgcgacgaggtgaataaaagagcccagagtactaacattttgtggacctgttggaaactaattgccgccgcaactcgactggaaccaagttcaagaccactgacaagcgccccggtccaaccagaacactaaacggagccgcacagcaagcaccacggacgatcgtcatcatccctatggccaacctgcatggatactcttgataacagttaagctatatttaccaacaccacctccgttcacgacataacatccccacaccatagctacacccaaagtcgctgtacccgtaccgcccgcaaaatggaagtccgacgacgaaaaatcgacgtgctcaaggcccagaaaaacggctacgaatcgggcccaccatctcgacaatcgtcgcagccctcctcaagagcatcgtccagaacccgcaacaaacactcctcgtccaccctgtcgctcagcggactgaccatgaaagtccagaagaaacctgcgggacccccggcgaactccaaaacgccattcctacacatcaagcccgtgcacacgtgctgctccacatcaatgctttcgcgcgattatgacggctccaaccccagcttcaagggcttcaaaaacatcggcatgatcattctcattgtgggaaatctacggctcgcattcgaaaactacctcaaatacggcatttccaacccgttcttcgaccccaaaattactccttccgagtggcagctctcaggcttgctcatagtcgtggcctacgcacatatcctcatggcctacgctattgagagcgctgccaagctgctgttcctctctagcaaacaccactacatggccgtggggcttctgcataccatgaacactttgtcgtccatctcgttgctgtcctacgtcgtctactactacctgcccaaccccgtggcaggcacaatagtcgagtttgtggccgttattctgtctctcaaactcgcctcatacgccctcactaactcggatctccgaaaagccgcaattcatgcccagaagctcgacaagacgcaagacgataacgaaaaggaatccacctcgtcttcctcttcttcagatgacgcagagactttggcagacattgacgtcattcctgcatactacgcacagctgccctacccccagaatgtgacgctgtcgaacctgctgtacttctggtttgctcccacactggtctaccagcccgtgtaccccaagacggagcgtattcgacccaagcacgtgatccgaaacctgtttgagctcgtctctctgtgcatgcttattcagtttctcatcttccagtacgcctaccccatcatgcagtcgtgtctggctctgttcttccagcccaagctcgattatgccaacatctccgagcgcctcatgaagttggcctccgtgtctatgatggtctggctcattggattctacgctttcttccagaacggtctcaatcttattgccgagctcacctgttttggaaacagaaccttctaccagcagtggtggaattcccgctccattggccagtactggactctatggaacaagccagtcaaccagtactttagacaccacgtctacgtgcctcttctcgctcggggcatgtcgcggttcaatgcgtcggtggtggttttctttttctccgccgtcatccatgaactgcttgtcggcatccccactcacaacatcatcggagccgccttcttcggcatgatgtcgcaggtgcctctgatcatggctactgagaaccttcagcatattaactcctctctgggccccttccttggcaactgtgcattctggttcacctttttcctgggacaacccacttgtgcattcctttattatctggcttacaactacaagcagaaccagtagcataacactcatcagtagcctttacagtgataaataatattgacattgacgtaatctgatgattgtagacaaacgtgagacggcattagcgataatattttgacgatgcccacgggcatataataaccattctgtaatagaactttgagcatgttcagaagtaatgagtttcatacataggagtacagatcgtggcgatgtgtaattaccggtaccgcatatgttgaacctgttcgtaaaaggtgccttcaacacagttttgttgccgtcttacctgtgtataagcgttcagcttctggtgtgtagcagaaggaggtgaagttgaggaacaaataatggagacttgcagcagactccgtgcctcaagcaaaaaagatgatccagcagaggatcgaactctgagcctcctcggcactcgtagattgaagccactttgtgagcgagacgtgataaccattacaccactagaccgggggtgaagatcagagcccgtattgtcgtattcaaagttaaaaatggggattttttatattcagaagtgtgtgatttattgaacgccaataacccggcttactacagtaggtatatagctgtaatgaatcattagaataaaaaaaaatccgcctcaaagtagctgaaatgtatccctagaataatgaggggaaaaaactgataaatcaggtgaaatacggcatttagggacaggtaagagttcaattgaatttatgtcgtattatttaagctgtcggagaaaacttgtgtgcttttttgtatggttttcgtgactgtgtacatttctgtacccctgattccgaacatcttatactgacacaagacattggagataaatcatacagtataaagtagctacgtcttacttgtacagtgcttttaattacaatatgccccactatacatacatgtgattcattttataaacccctcctcaggctaattacttctaatgcgtttcgtatatattatctatggaattacaaga

SEQ ID NO: 55 - protein, 526 aa, Yarrowia lipolytica
MEVRRRKIDVLKAQKNGYESGPPSRQSSQPSSRASSRTRNKHSSSTLSLSGLTMKVQKKPAGPPANSKTPFLHIKPVHTCCSTSMLSRDYDGSNPSFKGFKNIGMIILIVGNLRLAFENYLKYGISNPFFDPKITPSEWQLSGLLIVVAYAHILMAYAIESAAKLLFLSSKHHYMAVGLLHTMNTLSSISLLSYVVYYYLPNPVAGTIVEFVAVILSLKLASYALTNSDLRKAAIHAQKLDKTQDDNEKESTSSSSSSDDAETLADIDVIPAYYAQLPYPQNVTLSNLLYFWFAPTLVYQPVYPKTERIRPKHVIRNLFELVSLCMLIQFLIFQYAYPIMQSCLALFFQPKLDYANISERLMKLASVSMMVWLIGFYAFFQNGLNLIAELTCFGNRTFYQQWWNSRSIGQYWTLWNKPVNQYFRHHVYVPLLARGMSRFNASVVVFFFSAVIHELLVGIPTHNIIGAAFFGMMSQVPLIMATENLQHINSSLGPFLGNCAFWFTFFLGQPTCAFLYYLAYNYKQNQ

SEQ ID NO: 56 - DNA, 3947 bp, Yarrowia lipolytica
gagtcgactttccgatacagaagtggaaccagacttgctccacattcatctttcccccatttttctatagtggttcagtggagttgactacttgataacaattggtacatgtaatactgatacatctccgtggatataccatacacattcagtgaccaagcagcgtcgaacatgaccttcaagaatgggttccctgtcatctggagggatgacggctctggtttgtcaagtctgctgaatgcatctacttgtacgttgtagtaacttcctctgtcctccctagaaacgacccttcaatgcagcacagacgataaatcagcgtatatcgtccagtccaactgtgagacctacgatgacctagagaatggttctcttaatctggagtctgcttcttctccacacaactgacagcgtatcccccatggcaccgaagctccagtactgaccgacctttgctacaagtgctgtgtccagagttctctagtaggtacgatacgacagcagcttgatactgtgcttggacctacccttgcatgaggtcttgctgttgtccactgacacaggaccattctgcggggtccactgaaacaagaccattctgacgcgatttctttgtacagatgaagtcagaaatacaagcaatggcaccactcttttggttcgaccatgactgtttttacatccatttccagtaccaaccgcacttttttcatgttcgccccacatttattaatattatgctcttcatgcaccagcaaaataaccgaaacgcgcatatgatagtgggattctcgatttgcccggcagacaaacgccgctaaaatcgccacagtatcgaattttaattgaatacgaacgtcaattccggcttatccttctagcagttgtctcccgcagctcgctccatgactaatcattcacgcgacatgtctcagctaccccggtctggctcatgtaaaaaaagtgtaatcggcttttttccggttgatcacaaccatcaatgacacaacctgtgaatcggaaggcgactgtcgagcgggtcgagccagcagtggaggtggctgactccgagtccgaggccaagaccgacgtccacgttcaccaccatcatcaccaccacaagcgaaaatccgtcaagggcaagattctcaacttcttcacccgaagtcgacgtatcaccttcgtcctcggcgccgtggtcggtgtgatagccgcgggatactacgctgcgccaccggagctcagcattgatatcgatgctcttctcggcgacttgccctcgttcgactttgacgctctatctctcgacaacttgtccatggacagtgtgtcggactttgtacaagacatgaaatcgcggtttccgaccaagattctgcaggaggcggccaagatcgagaagcaccagaaaagcgaacagaaggctgccccttttgctgtgggcaaggctatgaagagcgagggactcaacgccaagtacccggtggtgctggtgcccggcgtcatctccacgggactggagagctggtccctggagggaaccgaggagtgtcccaccgagtcgcacttcagaaagcgaatgtggggctcctggtacatgatccgagtcatgctgctggacaagtactgctggctgcagaacctgatgctggacacagagaccggtctagaccctccccatttcaagctgcgagccgcccagggatttgcctccgccgacttctttatggcaggctactggctgtggaacaagctgctcgagaacctggctgttattggatacgatacggatacaatgtctgctgcggcgtacgactggagactgtcctaccctgatttggagcaccgagacggatacttctccaagctcaaagcttcaatcgaagagactaagcgtatgacaggtgagaagacagttctgacgggccattccatgggctcccaggtcatcttctacttcatgaagtgggctgaggccgagggatatggaggaggaggtcccaactgggtcaatgaccatattgaatcctttgtcgacatttccggctccatgctgggtactcccaagaccctggttgctcttctgtctggagaaatgaaggataccgtgcagctgaacgcgatggctgtgtatggactggagcagttcttctctcgacgagagcgagccgatctgctgcgaacatggggaggaattgcttccatgattcccaagggtggtaaggctatctggggtgatcattctggagcccctgatgacgagcctggccagaatgtcacctttggcaacttcatcaagttcaaggagtccttgaccgagtactctgctaagaacctcaccatggatgaaaccgttgacttcctgtattctcagtctcccgagtggtttgtgaaccgaaccgagggtgcttactcctttggaattgccaagactcgaaagcaggttgagcagaatgagaagcgaccttctacctggagcaaccctctggaagctgctctccccaatgcccccgatctcaagatctactgcttctatggagtcggtaaggataccgagcgagcctactactaccaggatgagcccaatcccgagcagaccaacttgaacgtcagtatcgctggaaacgaccctgatggtgtgcttatgggccagggcgatggaaccgtctcccttgtgacccataccatgtgtcaccgatggaaggacgagaattccaagttcaaccctggtaacgcccaggtcaaggttgtggagatgttgcaccagcctgatcgacttgatattcgaggcggtgctcagactgccgagcatgtggacattctggggcgttctgagttgaacgagatggttctgaaggtggccagtggaaagggaaatgagattgaagagagagtcatctccaacattgatgagtgggtgtggaagattgatctcggcagcaattagagagtccgttttgtagagtaatatgttttgtatatcacactgatggagaagggcgttcgtcctctcatgattccatgtggttgtttaatgaacacgtagaacgacgaacgacgaacgacgaacgacgaacgaacgagcaacaaacgtcatcaacgcgtgttttcccgcaaaatgaagaatgtttcccccacaatatgcgcctttctccagatctgcgcgttctgataaagaatatatttcaattgtttggaataaatcgggaaaattcaggagaaaagaggagaaacagggaaatcactgaaatctactcctgaaaatgttcgacaaaaaacgaattgcttccaatatacaaccactcctgtcacctcctcaacaaaatatattttactccgccaccaatgccaacccatggtgacgccaagattcacaagactgcttggagcggctgaaaagagtgttttgcggtcttctaagcgaccccgagccgctcaaagacccattggaggagatcttctttggccatttcggttctgttggctatcatccgcctccaaaaagcaaccccatttgcttctttttccaacccgttaaaatacctcatctcgcttcacattccagggtttcattttaagcgtagcaccatgcaaagtgcaggtgtagggcgtacaataaagcctgtgagacactctttgacaaggacaggacgctcgtatttttgagacgttgatgttgggggctagtggggcgatttgggggcaataaacggggtaaatctgtgcttttgaggctgtatttttgattccagccttttttaatttattgttatctgctttactacaaatttctgccgtatttttagcccctcaatttcgtctgtcacttctcatgtcgcacaaggctgtgccatcttgcattaggttatccaaccctactgtctcttctctgtctgtctggaatctgaaagcaagccgtgcgactttttgcaacttgcgaggtccgt

SEQ ID NO: 57 - protein, 648 aa, Yarrowia lipolytica
MTQPVNRKATVERVEPAVEVADSESEAKTDVHVHHHHHHHKRKSVKGKILNFFTRSRRITFVLGAVVGVIAAGYYAAPPELSIDIDALLGDLPSFDFDALSLDNLSMDSVSDFVQDMKSRFPTKILQEAAKIEKHQKSEQKAAPFAVGKAMKSEGLNAKYPVVLVPGVISTGLESWSLEGTEECPTESHFRKRMWGSWYMIRVMLLDKYCWLQNLMLDTETGLDPPHFKLRAAQGFASADFFMAGYWLWNKLLENLAVIGYDTDTMSAAAYDWRLSYPDLEHRDGYFSKLKASIEETKRMTGEKTVLTGHSMGSQVIFYFMKWAEAEGYGGGGPNWVNDHIESFVDISGSMLGTPKTLVALLSGEMKDTVQLNAMAVYGLEQFFSRRERADLLRTWGGIASMIPKGGKAIWGDHSGAPDDEPGQNVTFGNFIKFKESLTEYSAKNLTMDETVDFLYSQSPEWFVNRTEGAYSFGIAKTRKQVEQNEKRPSTWSNPLEAALPNAPDLKIYCFYGVGKDTERAYYYQDEPNPEQTNLNVSIAGNDPDGVLMGQGDGTVSLVTHTMCHRWKDENSKFNPGNAQVKVVEMLHQPDRLDIRGGAQTAEHVDILGRSELNEMVLKVASGKGNEIEERVISNIDEWVWKIDLGSN

SEQ ID NO: 58 - DNA, 3632 bp, Yarrowia lipolytica
gccgacaatctctcttcctcattaccgcgctttaatagcgaggagctattagtttgatttcatccaaaccccgcgggacgtattggcgggccaaccaaaaaaagagtcttcatttagcgcgaaaaaaacaagaggttgaaggcaatatttctgctcaattagacgacacatgatctcttgtccaacagcaatacggttcctggacgccgcgggtcacgtgaagggtatccccggcttgtggatctctggacacctgtggatctatcgaccctatggacttagctggtctgtgatcatgcgatgaggccaagacttttgtgcagatgaatcatgtgtctgggtgagacttgggtcggtcttgtagcaaggctggcggggcgccacaccgacggcgaggaagggggggttctcgttgactccggcctccgactgcgagctgtgaaatcgcgcgaccgttcgctggtgccttacagatgcatctaaaaaaagcctcgttgttgctgggtaagtcaaacctcggtaagcacgatgggctgcccagagagagcgagtgcactgcagcccggaaggttgactgaagaaatcttataccgtttatcttctttaatttttttgtcagcgcctttaaactcggcggcttaattagtgaccgttttgggcacctaaaatggcgcacgaggttattgttgaggtcagtaggtgtgagagagtctgagggcgggggccggacaagtgctcacttatatagttgtctcccatgacacgggtctggtaacacgccggcgcctgacacggctcagatttggttcagcttggcacaatttggtttcaagcttgcactttagcagtttgtacactggcagcaagtttgaatttcagtgcatgagaactctatgtataagatgcaaatgacgcacggccctacatatagcggggtctatcgtggcgacggggcaaaaaggatacaagtcgttcttacaagacactctccgcacacacaatggccacactccaccccgaagacgccgcaggacggcccgtgcgacgacgacctcgtccctccagttcgggcggctccagatcgccgtccaccaaacgacactcgatagtgcgggagcatctcggagaagagctcaatgtgcccgacggccaggaaatggacctgggccaggtcaacaagaacctcaatgccgcatacgccaaggccgagaaggactcggacgacgagaaggaaaagaaggaggagggcgtggtggacgagctgccagagaagtattcctaccctcgattctcaaagaacaaccgacgctacagattcaccgacatcaagttcaagccaacaccgtcgattctcgacaagttcgcccacaaggactcggagttctttggcttctacaccctgctgtggatggtgtttgccttctgcgtcttccgaaccggcctgctcaactacacaaacgaaggcatcctgttccggggccagattttcgccattctcagcaaagatctctggaaagtcgcattggtcgatctgggcatgtacctgaccacctatctgtctgtgtttctgcaattggccgtcaagcacggtctggtcgactggaactcgtttggctggatcatccagaacgtgcaccagaccctgttcctcttcttctacctttgggtcgccaagtcgagtaacctgccttggatcggtaacatcttcattgtgcttcatgcctttgtcatgctcatgaaacaacactcgtacgccttctacaatggctacctatggactgtcgaggacgagctctcccacgcaaagcagcgtctcaccgaagacattcctgtttcagagaaggaggatctcaagctggacatcgagttctgcgagacagagctcaaggtccaatccagacacacccctttccccaccaacatcaccttttctaactacttctggtactccatgttcccaacgctcgtctacgaaattgagttccctcgaaccccccgaatcaagtggacatacgtgctggagaaggtcgccgcagtctttggcgtcttcttccttatgatctgggtcgcagagtcgtacctgtatccccctgtggtggctgttattcaaatgcgagacgaacccttctggaacaaggtccgaatctatcccattttcctgtcggacattctgctgccctttgtcattgagtacatgcttgttttctacatcatctgggacgccattctcaacggcattgccgagctcactcgcttcgccgacagagacttttatggcccctggtggaactgtaccagctgggagcagtttagccgagaatggaacattcctgtctaccagttcctcaagcgacacgtctaccactcgtccatctctgctttcaagttctccaagggcgcagctaccctcaccaccttcttgctgtcttctcttgtccacgagctggtcatgtttgccatctttaagaagttccgaggatacctgctgttgctgcagatgacccagctgcccctggccatgctgcagaaaaccaaatggatccaggacagacccgtttttggcaacgctttcttctggttctcgctcatgatcggaccttctctcatgtgttccatgtacctcctcttctaagcacagtcgcttcaccacttgtgcctatagaatgtcaaccactgtcaacttcttgtatggccatcaactatgaacaagtcagttgacagctacaatgcttccactgaagttatcctcccccactgtattttattcttaacactgatcatctacctaacgagacactgtagagccactggtattgtatgaaacttcgccaagagatgccgacgatgaaagtctgagtttaagaaaatcaaaaaggggtaaaaaaaacggtttacgccctccgagccgatactacggagatatcggagttacagacactttgggagcaaagtctgcaatacaagcccacggaactagaggtgggtcgtcagtaaaggccgacgatactcagctggctgcataacttccggattgaaagagagccagtctttcgaacctcggctcagaaaagaagagaaggaaggtacaaacatcatacgatatagagtctttttagcacggcggtcagttggattgcaaagtggaagggcaagagtactgctgccttgtttaattggtatccactatgcattttgctttttgcaagtggaactgggagaacttgtagccttgggttgatgcggtcggagattagagttgatctgaaaaataaaaaataaaaaaacgagatgcggattgtggaagctgtagaggatatgtgactacaagagtagtatctacgagtgtgtacttgctgcgaaagttgatttcgaacgaaccagcttcattgactaaacagtgatgattttcgagacagcggaaaacttcaagccagtatgcagcatttaggctcatcggcacctattttagccgcaactagccgcttctttctgtaacatacgatgatttgtcaggtacagtacagtaccgttttaagcacctgaactagcggtggcctacgtgcacatgctcgtattgtagtaccgagttaaagattgttttggagtctagagttgtatttgtcattgtttta

SEQ ID NO: 59 - protein, 543 aa, Yarrowia lipolytica
MATLHPEDAAGRPVRRRPRPSSSGGSRSPSTKRHSIVREHLGEELNVPDGQEMDLGQVNKNLNAAYAKAEKDSDDEKEKKEEGVVDELPEKYSYPRFSKNNRRYRFTDIKFKPTPSILDKFAHKDSEFFGFYTLLWMVFAFCVFRTGLLNYTNEGILFRGQIFAILSKDLWKVALVDLGMYLTTYLSVFLQLAVKHGLVDWNSFGWIIQNVHQTLFLFFYLWVAKSSNLPWIGNIFIVLHAFVMLMKQHSYAFYNGYLWTVEDELSHAKQRLTEDIPVSEKEDLKLDIEFCETELKVQSRHTPFPTNITFSNYFWYSMFPTLVYEIEFPRTPRIKWTYVLEKVAAVFGVFFLMIWVAESYLYPPVVAVIQMRDEPFWNKVRIYPIFLSDILLPFVIEYMLVFYIIWDAILNGIAELTRFADRDFYGPWWNCTSWEQFSREWNIPVYQFLKRHVYHSSISAFKFSKGAATLTTFLLSSLVHELVMFAIFKKFRGYLLLLQMTQLPLAMLQKTKWIQDRPVFGNAFFWFSLMIGPSLMCSMYLLF

SEQ ID NO: 60 - DNA, 4034 bp, Yarrowia lipolytica
tgagtataatccgtctccgttcccacttgaggggaggactttggacgggtttcggtgcaacctcgtcaggtgaataatcatgattcatttgcgtgatctggctgggacaaaatacaagtgcttgtatatggtataccaggaggaccacttatgagaatgccaaagaaaattgaaggggaaaataaatgctgccacccgatactcgaaatataccattggacgtgttttccacataggtaatcgccactccatacccgagatctgaagtgaaacattcgtgttctgtagccgttagcccacccccacacactgagacactcggctgatgtttcgaaattcatgctactgtacggtacggacttaacacttatccccattgtcctgggactactacattgtagtatactctacacatccaacaagcaatactttttcgtacaagtaggtagttgtgcgagtactcgtacaatggaaaaacattatacatgaagcagggcgtgcgtcggtctgtagggtgaaatagggagccgacagtgcatttccagcattctagattgatctgaatcaataatttgtgggggaaattggcactagtgctttgtttattagctatttgacagtctgatgatgagaaagtcgttcttcggcttagcccaaccccacactcactacgcacctggcacagggtggtgcatataatatgcaagtacggagatgtactaacgctagccaggagagagtgacgaaacgtagccatccgttgagcgggcagacgtgggagtaatatagtcgtttagggcacgtgattgcgaccccagtggtatcatctttgtctcccaaactcttcgtaatgcccacccaggacgatcctctctgaactttactaccccacattcccgagactataaagtatagacggctcctacaaaagagagcaggtggaataacacactattgaccgacaatgacaaccaacacattcaccgatcctcctgtggagatggccaaggagcgaggtaagactcaattcactgtccgagatgtgaccaacttcctcaatggtggagaagaagagacccagattgtcgagaagatcatgagcagtattgaacgtgatccagtactgtctgtcactgctgactacgactgcaaccttcagcaggcccgaaaacagaccatggagcgggtggctgctctgtcgccttatctggtcaccgatactgagaagctatctctgtggcgtgcgcaactgcatggaatggttgatatgtctactcgtacgcggttgtcgatccacaacaacctgttcattggttccatcaggggatctggtactcctgaacagttcaagtactgggtcaagaagggagcggtggctgttaagcagttctatggatgctttgccatgacagagttgggccatggaagcaacctcaagggactagagacaaccgccacttatgaccaggacagtgaccagttcattatcaacactcctcatattggtgctaccaagtggtggattggcggtgcagcccacacttccacccattgtgtttgtttcgcgaaactgattgtgcatggcaaggactatggtactcgaaactttgtggtacctctccgaaatgtccacgatcacagtctcaaggtcggtgtttcaattggagacattggaaagaagatgggcagagatggtgttgacaatggctggatccagttcaccaatgttcgaatccccagacagaacatgctaatgagatatgccaaggtgtctgatactggagtggtaaccaaacccgctcttgaccaactcacttatggagccctcattcgaggtcgagtgtccatgattgccgactcgttccacgtctccaaacgattcctcacaattgctcttcggtacgcttgtgtccgacgacagtttggaacctctggagacactaaggagaccaagatcatcgactacccttaccaccagcgacgattgctgcctcttctggcctactgctacgctatgaagatgggtgctgatgaggctcagaagacttggattgagaccaccgatcgaattctggctctcaatcccaacgaccccgcccagaagaacgatctggagaaggccgtcaccgacacaaaggagctgtttgctgcgtctgcaggaatgaaggcatttaccacgtggggatgtgccaaaatcattgatgagtgccgacaggcctgtggaggtcatggatactctggatataacggatttggccagggctacgctgactgggttgtccagtgtacctgggaaggagacaacaacgttctgtgtctgtcaatgggccgagggctggttcagtcagctctacagattttggctggaaagcacgtcggtgcttctattcagtacgtaggagacaagtctaaaatctcccagaacggccagggtacccccagagagcaacttctgtcccccgagtttctagtagaagctttcagaacggcttctcgaaacaacattctcagaaccaccgataaataccaagagcttgtcaaaactctcaatcccgaccaggcctttgaggagctgtctcagcagagattccagtgtgctcgaatccacacacgacagcatcttatctcttcattctatgcccgaattgccactgccaaagacgatatcaagccccatctgctgaaactggccaatctgtttgccctctggtcaattgaggaggacactggaatcttcctgcgggagaacatcctcacccctggagacattgacctgatcaacagtcttgtggacgagctctgtgttgcagttcgagatcaggtaattggactcactgatgcctttggtctctctgacttcttcattaacgctcccatcggctcctacgatggtaatgtttacgaaaagtactttgccaaggtcaaccagcaaaaccccgctactaaccctcgtcctccctactacgagtcgactctcaagcccttcttgttccgagaagaggaggacgatgaaatttgcgatctcgatgagtgatgttagtaattatgagatacattctttgcaatgatgagggctcttgtagagtattctgctgtaaaggagatcttgaattttgttgttgaatttgtgtaattgtcttatgattattgtcttgtgtagcgcttttcaagcctgcaggaaaagtgaaatgaacatgttattgccatatcatccgttattggtgtttagttcctattgggcttgattaccaacgatccgggtcgaaagacccctccgtttaggggtggtaagtctcaagtggctgaacacgtgcatgcactgtacatcgccgagaccactcaacaccatcaagggctactgtattcgtagagcatacggcgttcaaggcttcaccgataattagagctcagcgcaatcaattatttgaaggcttaatggcactgtcagctgcttgatcttgcacgcaattactacaatatatagtccaacataataacataattagaattatgatcatccgtatcaatcatacacaaaatccttacacctaatctcgtagctcctccacaaatcccacaacgcgtcctgcctccacaattctaagctcctcatctgcagactcgtgcagaccaagccaggtagcgtcctgaacaggcagttcatttgtccaggtctggaaatcagccacacgcgagccagtagtcagcttgcatggaccgagagcacgcgactcgttgtcaaaataatccacctgcatcacctgttggaccatctcttgtaatcgagccaggtcagcagcgatgtcaatcttggcgctgtaaatgtcagacagcaccgagactacagcatgccaaggaatggaatggggagcaatattgtctctctgaccactcacatgacgaacccatccttcaatagctcgcaaactggcagccaggtctccctcatggaagccataatccttggcaaagcccagaggcacaaaacgagccaggtcaacaatgcagacatgcagccaactggccaggaagtt

SEQ ID NO: 61 - protein, 677 aa, Yarrowia lipolytica
MAKERGKTQFTVRDVTNFLNGGEEETQIVEKIMSSIERDPVLSVTADYDCNLQQARKQTMERVAALSPYLVTDTEKLSLWRAQLHGMVDMSTRTRLSIHNNLFIGSIRGSGTPEQFKYWVKKGAVAVKQFYGCFAMTELGHGSNLKGLETTATYDQDSDQFIINTPHIGATKWWIGGAAHTSTHCVCFAKLIVHGKDYGTRNFVVPLRNVHDHSLKVGVSIGDIGKKMGRDGVDNGWIQFTNVRIPRQNMLMRYAKVSDTGVVTKPALDQLTYGALIRGRVSMIADSFHVSKRFLTIALRYACVRRQFGTSGDTKETKIIDYPYHQRRLLPLLAYCYAMKMGADEAQKTWIETTDRILALNPNDPAQKNDLEKAVTDTKELFAASAGMKAFTTWGCAKIIDECRQACGGHGYSGYNGFGQGYADWVVQCTWEGDNNVLCLSMGRGLVQSALQILAGKHVGASIQYVGDKSKISQNGQGTPREQLLSPEFLVEAFRTASRNNILRTTDKYQELVKTLNPDQAFEELSQQRFQCARIHTRQHLISSFYARIATAKDDIKPHLLKLANLFALWSIEEDTGIFLRENILTPGDIDLINSLVDELCVAVRDQVIGLTDAFGLSDFFINAPIGSYDGNVYEKYFAKVNQQNPATNPRPPYYESTLKPFLFREEEDDEICDLDE

SEQ ID NO: 62 - DNA, 4103 bp, Yarrowia lipolytica
gattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaaccccaacaacactggcaccattgaaatcaacggtaaggagtacaacaccttcaccgagccccccgtggccatggctcaggagcgagccaagacctccttccccgtgcgagagatgacctacttcctcgacggtggcgagaagaacaccctcaaaaacgagcagatcatggaggagattgagcgagaccctcttttcaacaacgacaactactacgatctcaacaaggagcagatccgagagctcaccatggagcgagtcgccaagctgtctctgtttgtgcgtgatcagcccgaggacgacatcaagaagcgatttgctctcattggtatcgccgatatgggaacctacacccgacttggtgtccactacggcctcttctttggcgccgtccgaggtaccggaactgccgagcagtttggccactggatctccaagggagccggagacctgcgaaagttctacggatgtttctccatgaccgagctgggccatggctccaacctggctggtctcgagaccaccgccatctacgatgaggagaccgacgagttcatcatcaacacccctcacattgccgccaccaagtggtggattggaggagccgcccacaccgccacccacactgtcgtgttcgcccgactcattgtcaagggcaaggactacggtgtcaagacctttgttgtccagctgcgaaacatcaacgaccacagcctcaaggtcggtatctctattggtgatatcggaaagaagatgggccgagacggtatcgataacggatggatccagttcaccaacgtgcgaatcccccgacagaacctgctcatgaagtacacaaaggtcgaccgagagggtaacgtgacccagcctcctctggctcagcttacctacggttctcttatcactggtcgagtctccatggcctctgattctcaccaggtcggaaagcgattcatcaccattgctctgcgatacgcctgcattcgacgacagttctccaccacccccggccagcccgagaccaagatcatcgactacccctaccatcagcgacgacttctgcctcttctggcctatgtctatgctcttaagatgactgccgatgaggttggagctctcttctcccgaaccatgcttaagatggacgacctcaagcccgacgacaaggccggcctcaatgaggttgtttccgacgtcaaggagctcttctccgtctccgccggtctcaaggccttctccacctgggcttgtgccgacgtcattgacaagacccgacaggcttgcggtggccacggttactctggatacaacggtttcggccaggcctacgccgactgggttgtccagtgcacctgggagggtgacaacaacattctcaccctttctgccggccgagctcttatccagtctgccgttgctctgcgaaagggcgagcctgttggtaacgccgtttcttacctgaagcgatacaaggatctggccaacgctaagctcaatggccgatctctcaccgaccccaaggtcctcgtcgaggcctgggaggttgctgccggtaacatcatcaaccgagccaccgaccagtacgagaagctcattggcgagggtcttaacgccgaccaggcctttgaggttctgtctcagcagcgattccaggccgccaaggtccacacacgacgacacctcattgccgctttcttctcccgaattgacaccgaggctggcgaggccatcaagcagcccctgcttaacctggctctgctgtttgccctgtggtccatcgaagaggactctggtctgttcctgcgagagggcttcctcgagcccaaggatatcgacaccgtcaccgagctcgtcaacaagtactgcaccactgtgcgagaggaggtcattggctacaccgatgccttcaacctgtccgactacttcatcaacgctcctattggatgctacgatggtgacgcttaccgacactacttccagaaggtcaacgagcagaaccctgcccgagacccccgacctccttactacgcctctactctcaagcccttccttttccgagaggaggaggatgatgacatttgcgagcttgatgaggaatagacaagcgggtatttattgtatgaataaagattatgtattgattgcaaaaaagtgcatttgtagatgtggtttattgtagagagtacggtatgtactgtacgaacattaggagctacttctacaagtagattttcttaacaagggtgaaatttactaggaagtacatgcatatttcgttagtagaatcacaaaagaaatgtacaagcacgtactacttgtactccacaatgtggagtgggagcaaaaaaattggacgacaccggaatcgaaccggggacctcgcgcatgctaagcgcatgtgataaccaactacaccagacgcccaagaactttcttggtgattatggaatacgtggtctgctatatctcaattttgctgtaatgaatcattagaattaaaaaaaaaaccccatttttgtgtgattgtcggccaagagatggaacaggaagaatacgtgaacaagcgagcacgaatgccatatgctcttctgaacaaccgagtccgaatccgatttgtgggtatcacatgtctcaagtagctgaaatgtatttcgctagaataaaataaatgagattaagaattaaaaatattggaatatattttcctagaatagaaactttggattttttttcggctattacagtctgaactggacaaacggctgactatatataaatattattgggtctgttttcttgtttatgtcgaaattatctgggttttactactgtgtcgtcgagtatagagtggcctgactggagaaaatgcagtagtatggacagtaggtactgccagccagagaagtttttggaattgatacttgagtcatttttccattccccattccccattccaacacaatcaactgtttctgaacattttccaaaacgcggagatgtatgtcacttggcactgcaagtctcgattcaaaatgcatctctttcagaccaaagtgtcatcagctttgtttggccccaaattaccgcaaatacttgtcga

SEQ ID NO: 63 - protein, 700 aa, Yarrowia lipolytica
MNPNNTGTIEINGKEYNTFTEPPVAMAQERAKTSFPVREMTYFLDGGEKNTLKNEQIMEEIERDPLFNNDNYYDLNKEQIRELTMERVAKLSLFVRDQPEDDIKKRFALIGIADMGTYTRLGVHYGLFFGAVRGTGTAEQFGHWISKGAGDLRKFYGCFSMTELGHGSNLAGLETTAIYDEETDEFIINTPHIAATKWWIGGAAHTATHTVVFARLIVKGKDYGVKTFVVQLRNINDHSLKVGISIGDIGKKMGRDGIDNGWIQFTNVRIPRQNLLMKYTKVDREGNVTQPPLAQLTYGSLITGRVSMASDSHQVGKRFITIALRYACIRRQFSTTPGQPETKIIDYPYHQRRLLPLLAYVYALKMTADEVGALFSRTMLKMDDLKPDDKAGLNEVVSDVKELFSVSAGLKAFSTWACADVIDKTRQACGGHGYSGYNGFGQAYADWVVQCTWEGDNNILTLSAGRALIQSAVALRKGEPVGNAVSYLKRYKDLANAKLNGRSLTDPKVLVEAWEVAAGNIINRATDQYEKLIGEGLNADQAFEVLSQQRFQAAKVHTRRHLIAAFFSRIDTEAGEAIKQPLLNLALLFALWSIEEDSGLFLREGFLEPKDIDTVTELVNKYCTTVREEVIGYTDAFNLSDYFINAPIGCYDGDAYRHYFQKVNEQNPARDPRPPYYASTLKPFLFREEEDDDICELDEE

SEQ ID NO: 64 - DNA, 4103 bp, Yarrowia lipolytica
tagtgtttttgttggtttttatttgatttgttggtttaatatttattttattttaatatttattttattttatttcaattaattattttatttatttgcttaaatcaaatattttggaccaataaaaaccgacacagttttcagacatgtttgtagctcatatgtatcaggtgtggttgtggggtatcaaagccgaggttggcaaattttactggggccttcagaacggtaaaaatagaccaatcagaattctgaaaagcacatcttgatctcctcattgcggggagtccaacggtggtcttattcccccgaatttcccgctcaatatcgttccagaccgacccggacacagtgcttaacgccgttccgaaactctaccgcagatatgctccaacggactgggctgcatagatgtgatcctcggcttggagaaatggataaaagccggccaaaaaaaaagcggaaaaaagcggaaaaaaagagaaaaaaaatcgcaaaatcggaaaaatagggggaaaagacgcaaaaacgcaaggaggggggagtatatgacactgataagcaagctcacaacggttcctcttatttttttcctcatcttctgcctaggttcccaaaatcccagatgcttctctccagtgccaaaagtaagtaccccacaggttttcggccgaaaattccacgtgcagcaacgtcgtgtggggtgttaaaatgtgggggggggaccaggacaagaggctcttgtgggagccgaatgagagcacaaagcgggcgggtgtgataagggcatttttgcccattttcccttctcctgtctctccgacggtgatggcgttgtgcgtcctctatttctttttatttctttttgttttatttctctgactaccgatttggtttgatttcctcaaccccacacaaataagctcgggccgaggaatatatatatacacggacacagtcgccctgtggacaacacgtcactacctctacgatacacacaatgatctcccccaacctcacagctaacgtcgagattgacggcaagcagtacaacaccttcacagagccacccaaggcgctcgccggcgagcgagccaaggtcaagttccccatcaaggacatgacggagtttctgcacggtggcgaggagaacgtgaccatgatcgagcgactgatgacggagctcgagcgagaccccgtgctcaacgtgtcgggcgactacgacatgcccaaggagcagctgcgagagacggccgtggcgcgaattgcggcgctgtccggccactggaagaaggacacagaaaaggaggcgctgctgcggtcccagctgcacggcattgtggacatgggcacccgaatccgactcggtgtgcacacgggcctgttcatgggcgccatccggggttccggcaccaaggagcagtacgactactgggtgcgaaagggcgccgcggacgtcaagggcttctacggctgctttgctatgaccgagctgggccatggctccaacgtggccggtcttgagaccaccgccacctacatccaggacacggacgagttcatcatcaacacccccaacactggagccaccaagtggtggattggaggagccgcccactcggccacccacaccgcctgctttgctcgtctgcttgtcgacggcaaggactacggcgtcaagatctttgttgtccagctgcgagacgtctcttctcactctctcatgcccggcatcgctctcggcgacattggaaagaagatgggccgagacgccatcgacaacggctggatccagttcaccaatgtgcgaatcccccgacagaacatgctcatgaagtacgccaaggtctcgtctaccggcaaggtgtcgcagcctcctctggcccagctcacctacggcgctctcattggcggccgagtcaccatgattgccgactccttctttgtctcccagcgattcatcaccattgctctgcgatacgcctgtgtgcgacgacagtttggcaccacccccggccagcccgagactaagatcatcgactacccctaccatcagcgacgtctgctgcctcttctggccttcacctacgccatgaagatggccgccgaccagtcccagattcagtacgatcagaccaccgatctgctgcagaccatcgaccctaaggacaagggcgctctgggcaaggccattgtcgacctcaaggagctgtttgcctcttctgctggtctcaaggccttcaccacctggacctgtgccaacatcattgaccagtgccgacaggcctgcggtggccacggctactctggctacaacggctttggccaggcctacgccgactgggttgtccagtgcacctgggagggtgacaacaacgtcctgtgtctgtccatgggccgaggtctcatccagtcgtgtctgggccaccgaaagggtaagcctctgggctcttctgtcggctacctggctaacaagggtcttgagcaggctactctgagcggccgagacctcaaggaccccaaggttctcatcgaggcctgggagaaggtcgccaacggcgccatccagcgggccactgacaaatttgtcgagctcaccaagggcggcctctctcctgaccaggcctttgaggagctgtcgcagcagcgattccagtgtgccaagatccacacccgaaagcacctggtgactgccttctacgagcgaatcaacgcctctgcgaaggccgacgtcaagccttacctcatcaacctcgccaacctcttcactctgtggtccattgaggaggactctggtctcttcctgcgagagggtttcctgcagcccaaggacattgaccaggtgactgagctggtgaaccactactgcaaggaggttcgagaccaggttgccggctacaccgatgcctttggtctgtctgactggttcatcaacgctcccattggaaactacgatggtgacgtttacaagcattactttgccaaggttaaccagcagaaccctgctcagaacccccgacctccttactatgagagcactcttcgacctttcctgttccgagaggatgaggatgacgacatttgcgagctggacgaggaatagatggagcgtgtgttctgagtcgatgttttctatggagttgtgagtgttagtagacatgatgggtttatatatgatgaatgaatagatgtgattttgatttgcacgatggaattgagaactttgtaaacgtacatgggaatgtatgaatgtgggggttttgtgactggataactgacggtcagtggacgccgttgttcaaatatccaagagatgcgagaaactttgggtcaagtgaacatgtcctctctgttcaagtaaaccatcaactatgggtagtatatttagtaaggacaggagttgagagaggaaagttgccattctttggagtcccagaaacgtattttcgcgttccaagatcaaattagtagagtaatacgggcacgggaatccattcatagtctcaattttcccataggtgtgctacaaggtgttgagatgtggtacagtaccaccatgattcgagataaagagcccagaagtcattgatgaggtcaagaaatacacagatctacagctcaatacaatgaatatcttctttcatattcttcaggtgacaccaagggtgtctattttccccagaaatgcgtgaaaaggcgcgtgtgtagcgtggagtatgggttcggttggcgtatccttcatatatcgacgaaatagtagggcaagagatgacaaaaagtatctatatgtagacagcgtagaatatggatttgattggtataaattcatttattgcgtgtctcacaaatactctcgataagttggggttaaactggagatggaacaatgtcgatatctcgacatattttgatatttgtactgttgatagtgataaaaagtagaccgttcgaatctcgacaaggagaagagtccaatgaaataggttccatcatcattcgtcatagttaaacgccgctggttgccattactatccgtcttgactacaaccccaactcagcctagaccacagcgaagagaatcagtttggagaccgaaaatgagc

SEQ ID NO: 65 - protein, 700 aa, Yarrowia lipolytica
MISPNLTANVEIDGKQYNTFTEPPKALAGERAKVKFPIKDMTEFLHGGEENVTMIERLMTELERDPVLNVSGDYDMPKEQLRETAVARIAALSGHWKKDTEKEALLRSQLHGIVDMGTRIRLGVHTGLFMGAIRGSGTKEQYDYWVRKGAADVKGFYGCFAMTELGHGSNVAGLETTATYIQDTDEFIINTPNTGATKWWIGGAAHSATHTACFARLLVDGKDYGVKIFVVQLRDVSSHSLMPGIALGDIGKKMGRDAIDNGWIQFTNVRIPRQNMLMKYAKVSSTGKVSQPPLAQLTYGALIGGRVTMIADSFFVSQRFITIALRYACVRRQFGTTPGQPETKIIDYPYHQRRLLPLLAFTYAMKMAADQSQIQYDQTTDLLQTIDPKDKGALGKAIVDLKELFASSAGLKAFTTWTCANIIDQCRQACGGHGYSGYNGFGQAYADWVVQCTWEGDNNVLCLSMGRGLIQSCLGHRKGKPLGSSVGYLANKGLEQATLSGRDLKDPKVLIEAWEKVANGAIQRATDKFVELTKGGLSPDQAFEELSQQRFQCAKIHTRKHLVTAFYERINASAKADVKPYLINLANLFTLWSIEEDSGLFLREGFLQPKDIDQVTELVNHYCKEVRDQVAGYTDAFGLSDWFINAPIGNYDGDVYKHYFAKVNQQNPAQNPRPPYYESTLRPFLFREDEDDDICELDEE

SEQ ID NO: 66 - DNA, 4706 bp, Yarrowia lipolytica
acgacgtacaagccaaagagatgttgactccagcagcgaattagagtaagactacagtactggtagaagtaggcctattggactgtagggagtactgcaatgaccagtgttttcggtataaatatgagaattaccgagaaaaggtaaaaaatctgaatttcggagttacgatgggtggcgttccctagtggaacatagatagacctacaattgatgggctactctgtcgcaattgtcatttctgtcactattaccacatgtgtcaagtggcagcaattgtctagtaatcatttccgttgtcgcaggccaacggctgatttgtgtaatgtggggttgagttccagcacggggcttatttggattggatgagctaatgcaagccagagtgagcatgaaaacgggcagatccccatggggaggttggccaaccccgagccaacctctttggggagagatggggaaaccctccaagaagaggagaaggatttcgtaggtcatgccgtttcccagtcagaattatttaaacgtaaaatcggttataaccatttcctggttcatgttgaaatttccacaagcttctgtatggatgtggggttctacaactttgaaattgatgacattttcgagaaaatgagaacaaccgaatatttgtactccatattgatacggaaaaattcacaatggaatcacacaagatagctctgaacaattgaggacccttttctccctaaattcattgtggttgcggttgtattgccaacatggattacgcccgcccgatgacgagcgacttgcttgagccgaggcagagtctaaatcgggacaacgaaaccataaaccccataggaagccaaagtgaccactgcatgaccccagtatataacctgccccacccctccatacttgctgctttctagatcaagttataccggacctgctaactcaattgaggtagaagatacgttattaacacaatctatttattcaacacacacacacaatgtctggagaactaagatacgacggaaaggtcgtcattgttaccggtgccggtggcggtctcggtaaggcatacgcccttttctacggctctcgaggagcctctgttgttgtcaacgatcttggtggcgacttcaagggcgacggtgcccaggctggcagtggcaagcgagttgccgatgttgtcgtcgacgagattgtttccaagggaggcaaggctgttgctaactacgactctgtcgagaacggtgacaagattgtcgagactgccgtcaaggcttttggctccgtccacattgtcatcaacaacgccggtattctccgagatatttccttcaagaagatgaccgacaaggactgggatcttgtctacaaggtccacgttttcggtgcctacaaggttacccgagctgcctggccttacttccgaaagcagaagtacggtcgagttatctctacctcttccgctgctggtctttacggaaacttcggccagaccaactactccgctgccaagctcgccctggttggtttcggtgagactctcgccaaggagggtgccaagtacaacattacttccaacgtcatcgctcctcttgctgcttcccgaatgaccgagacagtcatgcccgaggatatcctcaagctcctcaagcctgagtacgttgttcctctggtcggctacctcacccacgactctgtcaccgagtcttatggtatttacgaggtcggtgctggttacatggctaaaatccgatgggagcgaggcaacggtgctgttttcaagggcgacgacactttcaccccgtctgctattctgaagcgatgggatgaggtcacctcttttgagagccccacctaccctaacggccctgctgacttcttcaaatacgctgaggagtctgttaagcgacccgagaacccccagggacccaccgtctccttcaaggaccaggttgtcattgtcactggagccggtgctggcattggccgagcttactctcacctccttgctaagcttggtgccaaggtcgttgttaacgatttcggtaaccctcagaaggttgtcgatgaaattaaggccctcggtggtatcgccgtcgctgacaagaacaacgtcatccacggtgagaaggttgttcagaccgctatcgacgccttcggtgctgtccacgccgttgtcaacaacgctggtattctccgagacaagtctttcgccaacatggatgatgagatgtggcagctgatctttgatgtccacctcaacggtacttactccgttaccaaggccgcgtggccccacttccttaagcagaagtacggccgtgtcatcaacaccacctcaacttctggtatctacggtaacttcggccaggccaactactctgccgccaaggctggtatcctcggtttctcccgagctcttgctcgagagggtgagaagtacaacattcttgtcaacaccattgcccctaacgctggtactgccatgactgcttctgtcttcactgaggagatgctcgagctcttcaagcccgatttcatcgcacccatcaccgtcctgcttgcttccgatcaggctcccgtcaccggtgatctgtttgagactggttctgcttggatcggacagactcgatggcagcgagctggtggtaaggccttcaacaccaagaagggtgtcacccccgaaatggttcgagacagctgggctaagatcgtcgacttcgatgatggtaactccacccatcccaccactccctccgagtctactactcagattcttgagaacatcttcaacgtgcctgatgaggaggttgaggagactgctctcgttgctggtcccggtggtcccggtatcctcaacaaggagggcgaacctttcgactacacttacacttaccgagacctcattctttacaaccttggtctcggtgccaaggctaatgagctcaagtatgtcttcgagggtgatgatgacttccagaccgtgcccactttcggtgttatcccttacatgggtggcctcatcactaccaactatggcgacttcgttcctaacttcaaccctatgatgcttctccacggtgagcagtaccttgaaatccgacagtggcctattcctaccaatgctacattggagaacaaggctaaggtcatcgatgtcgttgacaagggcaaggctgccctccttgtcactgctaccaccaccacgaacaaggagactggtgaggaggttttctacaacgagtcttctctcttcatccgaggctctggtggtttcggtggtaagtctaccggtactgaccgtggcgctgccactgctgccaacaagccccctgctcgagctcctgacttcgttaaggagatcaagatccaggaggaccaggctgccatttaccgactttctggtgattacaaccctcttcacatcgaccctgcttttgctgctgttggtaactttgaccgacctattctccacggtctctgctcttttggtgtctccggtaaggctctttacgatcagtttggtcctttcaagaacgctaaggtccgatttgctggtcacgtcttccctggtgagaccctgaaggttgagggctggaaggagggcaacaaggtcattttccagaccaaggttgttgagcgaggtactaccgccatcagcaatgccgccattgagctcttccccaaggatgctaagctctaagctattatctgaccaagtgatacgagttaagtaaattttaagaaatgtatgagttaagattagttaatactgttatagtttcagttaggaccaccaccataaaaaaaaacgtagagtaatgtatcgcgctgtgtactgcaagtacagtacatactgtaccggaagtgtgagatcagtagagggtctgatgtgttaatagttcaacaaagtgttgttcaagtactgtacgaatacttgtactgtattatgtgattataaaatatatatattggatctcgccccctcccccaatataatgttattacaggtataaaaatttttatggatgaaaaaataaaataaaacaagttgcgatcaagagacttcgtggtgggacttttttattactaacaacctaaattgatcgtgcataatcgtgttgaattctcacacatcgcgccaaatcgacagctttcatgaacagagtgaatcaacaagaagcggaccagaaccctgaggctactctttatataggcaaccttgacgagaacgtcacagaagcgattctgtatgaacttatgcttcaagcgggccctatcgtgcacatcaatcttcctaaagacagagtcagccagacacaccaaggctatggcttcgtggagtacaaaacagaggctgatgcaaattacgcagcgtctattatgaaccaaatctggctgttcggaaaatccattcgagtcagcaaatctgcatccgacaagcaaaacgggtttgagattggagcaacacttttcgtgggaagtttggatcctctcgtagatgagtctacactacagcaaacgttctctgtgtttggaccatttgccaaaccccctcgaatatctcgtgacacagatggttcttcaaaagggtacggttttgtgtcttttactgattttgaacattcagaccgtgcattggaatcaatggacaaacagtatctcatgaaccagcaaataacagtcacatatgccttcaagca

SEQ ID NO: 67 - protein, 901 aa, Yarrowia lipolytica
MSGELRYDGKVVIVTGAGGGLGKAYALFYGSRGASVVVNDLGGDFKGDGAQAGSGKRVADVVVDEIVSKGGKAVANYDSVENGDKIVETAVKAFGSVHIVINNAGILRDISFKKMTDKDWDLVYKVHVFGAYKVTRAAWPYFRKQKYGRVISTSSAAGLYGNFGQTNYSAAKLALVGFGETLAKEGAKYNITSNVIAPLAASRMTETVMPEDILKLLKPEYVVPLVGYLTHDSVTESYGIYEVGAGYMAKIRWERGNGAVFKGDDTFTPSAILKRWDEVTSFESPTYPNGPADFFKYAEESVKRPENPQGPTVSFKDQVVIVTGAGAGIGRAYSHLLAKLGAKVVVNDFGNPQKVVDEIKALGGIAVADKNNVIHGEKVVQTAIDAFGAVHAVVNNAGILRDKSFANMDDEMWQLIFDVHLNGTYSVTKAAWPHFLKQKYGRVINTTSTSGIYGNFGQANYSAAKAGILGFSRALAREGEKYNILVNTIAPNAGTAMTASVFTEEMLELFKPDFIAPITVLLASDQAPVTGDLFETGSAWIGQTRWQRAGGKAFNTKKGVTPEMVRDSWAKIVDFDDGNSTHPTTPSESTTQILENIFNVPDEEVEETALVAGPGGPGILNKEGEPFDYTYTYRDLILYNLGLGAKANELKYVFEGDDDFQTVPTFGVIPYMGGLITTNYGDFVPNFNPMMLLHGEQYLEIRQWPIPTNATLENKAKVIDVVDKGKAALLVTATTTTNKETGEEVFYNESSLFIRGSGGFGGKSTGTDRGAATAANKPPARAPDFVKEIKIQEDQAAIYRLSGDYNPLHIDPAFAAVGNFDRPILHGLCSFGVSGKALYDQFGPFKNAKVRFAGHVFPGETLKVEGWKEGNKVIFQTKVVERGTTAISNAAIELFPKDAKL

SEQ ID NO: 68 - DNA, 3134 bp, Yarrowia lipolytica
tgtgttttcgtcttagcgtcatgtattctcaagcttagtcagagagaaggactatggaggagaaggggagaattgagaagggtatttgaagggactttgaaggtcgcgtggaagaggtacttgaagaggtatttgaaggtcacgtggaagaggtatttgaagatcacgtggaagaagtacttgttttacagagaatatcggggtgattttgacagtgggattgtctcccaagtcctaatcgtttgacatgggagcagtgaaaagtcgggctaaaaaagggaatatcggaaatcggaaagacggaaagaattactggactcatgtttagtagatctgagcacttcaaatttgaaaatatctcttcaaacagcagatcggttggtcgtggaggtaccatcaagggtaaaatcaaggctatcatcaagggccatatatcgcaagtttgggggaagataatatgttcatagtgaatcagggttgtggatttcctcatctaacggcattgtaactagtcctggagggtcttttttatggataacctccatgtacgatgtatccaagatctccacgtactgtgttctgtttcctaagtaatacccaacaacctctccaacaaacacttgggaagatgcacttgtgctgagatgtcaagatgttagtactgtactggatggagagaatattaataaataattgttacccaactacatcttgtcgattgaaagagatacccctaagacagataggatatctgcaacccgaggaatgaaccccccagcaccggcaccctttctattaacaaaatgccaactgaaatttgaaaagttcaactaaacttatttgacccacaaaaactcgtcaaaagtggcggcgaaagctggcaaatgatgacatccccttggaactatgatatcccctcggaatcttcgtccccatttgccacatctacttgcaacgccacgtctgcttactaagcaacccaaatctgcctcggctcaaaatgtggggaagttcacatgcattcgctggtgaatctgatctgacactacaactacacaccaggtccaacatgagcgacaatacgacaatcaaaaagccgatccgacccaaaccgatccggacggaacgcctgccttacgctggggccgcagaaatcatccgagccaaccagaaagaccactactttgagtccgtgcttgaacagcatctcgtcacgtttctgcagaaatggaagggagtacgatttatccaccagtacaaggaggagctggagacggcgtccaagtttgcatatctcggtttgtgtacgcttgtgggctccaagactctcggagaagagtacaccaatctcatgtacactatcagagaccgaacagctctaccgggggtggtgagacggtttggctacgtgctttccaacactctgtttccatacctgtttgtgcgctacatgggcaagttgcgcgccaaactgatgcgcgagtatccccatctggtggagtacgacgaagatgagcctgtgcccagcccggaaacatggaaggagcgggtcatcaagacgtttgtgaacaagtttgacaagttcacggcgctggaggggtttaccgcgatccacttggcgattttctacgtctacggctcgtactaccagctcagtaagcggatctggggcatgcgttatgtatttggacaccgactggacaagaatgagcctcgaatcggttacgagatgctcggtctgctgattttcgcccggtttgccacgtcatttgtgcagacgggaagagagtacctcggagcgctgctggaaaagagcgtggagaaagaggcaggggagaaggaagatgaaaaggaagcggttgtgccgaaaaagaagtcgtcaattccgttcattgaggatacagaaggggagacggaagacaagatcgatctggaggaccctcgacagctcaagttcattcctgaggcgtccagagcgtgcactctgtgtctgtcatacattagtgcgccggcatgtacgccatgtggacactttttctgttgggactgtatttccgaatgggtgagagagaagcccgagtgtcccttgtgtcggcagggtgtgagagagcagaacttgttgcctatcagataatgacgaggtctggatggaaggactagtcagcgagacacagagcatcagggaccagacacgaccaattcaatcgacaacactgtgctgcatagcagtgcacagaggtcctgggcatgaatatattttagcattggagatatgagtggtagagcgtatacagtattaattgtggaggtatctcgtcgcattgatagagcaatacagttactgctgaagggaatgataccgagtatttcggcccgattcagttcttgatatcgtcattttgtctctattgtctacttttcagataacctcaacaaatcttcaacaaatctcccagtaaacagtcagagatcatatccgagatcatatcagatatgtcacgatccgagtacaataatggatattaatctgcttgattttgaattctgttgcgattatgatttctttgatttcgatatgaacacatacggcgactcccagacctttagaagctccagtttggattcttagcaatggttacactcaactatatcccaagtaatacttggtaacaatatgccaagttagtcattcattcgttataggagttagcaagtgtttgtcagctaaaaatggttagtcggtcgattaccacttagatcttttcagcgtggaacttgatggtacgcttgaaccgacacttggagtagtcggggctgttgatgacgtagatgacgtttcgctcagggtgaggagtgcaatagtagtactccttggggccgtctctcagctcaaaggttccatcggcggcaatgtcaaagaccgagccctggagcttgtagccgtagtcgccggtccagaacaaagcctgcagctccagataggcgatgggcatgtcgttaacagagaaggtgttgccctcgccctcggtgatggtgatgggttcgccgtcggtggaggcggtgatcaggtcatcttggtaggtgacgggcagagattcgaccgattgggcgtctgatctggtataggtcagctt

SEQ ID NO: 69 - protein, 377 aa, Yarrowia lipolytica
MWGSSHAFAGESDLTLQLHTRSNMSDNTTIKKPIRPKPIRTERLPYAGAAEIIRANQKDHYFESVLEQHLVTFLQKWKGVRFIHQYKEELETASKFAYLGLCTLVGSKTLGEEYTNLMYTIRDRTALPGVVRRFGYVLSNTLFPYLFVRYMGKLRAKLMREYPHLVEYDEDEPVPSPETWKERVIKTFVNKFDKFTALEGFTAIHLAIFYVYGSYYQLSKRIWGMRYVFGHRLDKNEPRIGYEMLGLLIFARFATSFVQTGREYLGALLEKSVEKEAGEKEDEKEAVVPKKKSSIPFIEDTEGETEDKIDLEDPRQLKFIPEASRACTLCLSYISAPACTPCGHFFCWDCISEWVREKPECPLCRQGVREQNLLPIR

SEQ ID NO: 70 - DNA, 3740 bp, Yarrowia lipolytica
tgaagcgggaaatcaagattgagagttggagtggtgggggcggtggtggaagagacagaggtgatagaagggaaagaaacgacagaagagacgcaaaaggaagaaacagaagagatggagataggaggaatggcgatcgaagagacagagacagagatagagatagagacgacaggtcaagtcgttcaagtcggtacaaccgatcaagatcaagatctccgactgtcaaaaaggaaacagatgaatatggccgggaccggaaggactaactgcattatatataaatatagattcttgattagtgatcaaagcatgagatactgtcaaggcaagcaagccttacaatatctgttggcttagttaaatcttttttgagatgtaataattggggaacctctgttagatgaacagttgcggcttactcatcttagacatggtccagtttaggcgtaattaattatcagttttaatgcgcctctacgtaataagcgttgccacaagttacagtagagatgataccattgctacaggaccgtatagcgtatataatgacgaggctaattgatgatgatcgacagcgaagacatatctacgtcaatctgacgcttcattttgtgcttcttgttcggatttgtgtttctcccagtgcacccagttttttgctgaccattttttgcttaccagtttttgctcgcgcgttttctcgattgaactctgctgcgagggtggtgtaagacacactccctgtagaacatgcatttcgaggtgtgacatttccatctctgttaaatcaaacgagtgacacaaacggaataaatctacatatacgtcgaaccctcttcttctcttctctctcttccttaatagacaccctcgctatcgactctgctcctccttatcacaccatcaaagtttagggtgtgtcccaacaccacctccacaccacactctattatcaccacctccacgactacataccactccttccaccacctcacaaatggcgaccgaacacgtggaacacgccgaaaacgccaagcagtcgccccaaccgtccttctcgggctccgagagcggccgaattggccggtaccaaatcatcaagactctgggagagggctcctttggcaaggtcaagctcgcctaccatctggccacccacgaaaaagtagcgctcaaaatcatcaacagaaagacgctggccaagtcggatatgcagggccgagtcgagcgggaaatctcgtacctgcgactgctcagacacccccacatcatcaagctgtacgacgtgatcaagtccaaggatgaaatcatcatggtgattgaatttgccggcaaggagctctttgactacattgtgcaacgtggaaagatgcccgaagacgaggctaggcgattcttccaacaaatcatctccgcagtcgagtactgccatagacacaagattgtgcacagagacctcaagcccgagaacctgttgctcgacgaaaacctcaacgtcaagattgccgactttggactctccaacattatgaccgatggtaacttcctcaagaccagttgtggctctcccaactatgctgctcccgaggttatcagtggcaagctgtatgctggccccgaggtcgacgtgtggtcctgtggtgtcattctatacgtcatgctgtgcggccgtctcccctttgacgacgaattcatccccaacctcttcaagaagatctccaatggtgtctacaccattcccccctatctgtctgccggcgcaaaacacctgcttcaacagatgctggtggtcaaccccctgaaccgaatcaccgtccagggcatcatggaagacccctggtttaaacagggcattgccgagtaccttgttccacgtgacctgaagcctgatcaggtggaaatcgacgacaaggttgtgggtgctctttcagacaccatgggctacgaccgagaccaggtctacgaggctctcaaggctcctaaatccgacggaaccgaaatccgagatgcctacgacctcatgaaggagaacagacagcagttggagaaggactcgaccgtgttcgacaaggtggacgaatccggcaagcctaagcaccagttctcgcgaaagaacccgccggctctgacgtttgactcacatcacatgcccaccattgtcgaccattctgatcttcgaacccctaacacaaccattgctgttcttccctcctcgctgccagcataccatcgggccaacatgatggcccatggtcccgctaccctgcgtaagctgaatcccatctcatcgcgaaaatcaaagacccgatggcatttcggcattcggtccaagtcgtaccctctcgacgtgatgggtgagctctaccgagctctcaagaacctgggagccgagtgggcaaagccatcagagaacgacctgtggaccatcagggtgcgatggagatacacccctgaaaaggatgagtcggacagcacaactaagccagacagcggactgcttaagatgcagattcaactgtatctgctggagcagaactcgtacttggtggacttcaagtttgacggctgggagtgcgagacggagagcggtgagattaagacccgcgatgtcgcccaggtgtcgaattcaaagtccgccaacgagaccttcacgttttccgcataccccttcttgcatctggccacccggctcattatggagctggccgtgagttctcagaaggagagtgagaagtaagcacttgtagagcacactagggatttagaggggattatggcacgtacaatatagataattaagcagtagctgagtcagttgaatgatcagaggtgtaacatgagtgtggatggattgtgtagagtcgttgtttaaaataatgagttaagaataattatacgactacaggatacgatgtacttgtattgtatcgatacagtacatacagtacatacgtgtaacatactcctaaactgttgcatcacctacaactccaactagtcggtcataattcattaatacgtttccctggtgtagtctaggccaaacactccttggtgaacttcttgagaaagattcgctctctagactccttgaattctctaggctggatttcggcccaaaaagaacccacaaacggcgcaatactcttcttgtagccggaatagttgtctcgaatgacaatatccgggttggcagaccgctgctgatgctccttgacgatgatactctcaaagtggtccacggtagacatgacaataagagccgacgaggttttgctcttgtagttggcgatgagcgttttaagtgccccagagttctccaccaccttgtggatcagctgatgcttttgttcctcgctgagtccttccgaggccagggggatagccaagcaggtgagaatagcgcctgtgtctcgtttagaagaggtgctgtttgtatcatccgcatgcttgttgatgaactgaactaaattcagtagcgtgctccacagctcattccagtggtaatttgagggagtgtccctgtaaaccatgagtgttgcttcagtgacgaccagagcgagatcataaatgtctgggagaggtttcttcatattgtatctgagacagcaaagaagtgcgtccaaaaggccggttccaaaggtgtaggcctctgtttctggaagggggggtttccgttgcttggcgttgattttaatagatgcttttgtgttgaacgactggtgcaattcgggcag

SEQ ID NO: 71 - protein, 579 aa, Yarrowia lipolytica
MATEHVEHAENAKQSPQPSFSGSESGRIGRYQIIKTLGEGSFGKVKLAYHLATHEKVALKIINRKTLAKSDMQGRVEREISYLRLLRHPHIIKLYDVIKSKDEIIMVIEFAGKELFDYIVQRGKMPEDEARRFFQQIISAVEYCHRHKIVHRDLKPENLLLDENLNVKIADFGLSNIMTDGNFLKTSCGSPNYAAPEVISGKLYAGPEVDVWSCGVILYVMLCGRLPFDDEFIPNLFKKISNGVYTIPPYLSAGAKHLLQQMLVVNPLNRITVQGIMEDPWFKQGIAEYLVPRDLKPDQVEIDDKVVGALSDTMGYDRDQVYEALKAPKSDGTEIRDAYDLMKENRQQLEKDSTVFDKVDESGKPKHQFSRKNPPALTFDSHHMPTIVDHSDLRTPNTTIAVLPSSLPAYHRANMMAHGPATLRKLNPISSRKSKTRWHFGIRSKSYPLDVMGELYRALKNLGAEWAKPSENDLWTIRVRWRYTPEKDESDSTTKPDSGLLKMQIQLYLLEQNSYLVDFKFDGWECETESGEIKTRDVAQVSNSKSANETFTFSAYPFLHLATRLIMELAVSSQKESEK

SEQ ID NO: 72 - DNA, 7490 bp, Yarrowia lipolytica
gcgtccaaaaaattcaaatttcgacatttttcggcttaaaatagaacatctacaagacgatgttgcagactcctgagtctgggaaaaggtggttgaggaacctacactggagctgcgattggagagcttcgtggcggccgtgggccaagattgggcagagtatgggtcgagcatttctattgcttctactgtacttgtacttgtacttgtacttacttacttgtacaagtacagacaggaggcttgctatttcgatgaaactaaagcacgtttgttatgtaccgaagttcggaacgaggctgtggagatggcaagtagttaagtgtcattaattggagtaggttaaaacatcaactgtgctcgttctaactgcttactgcgcctgttataccaccgctagcgccctctaacggatcctaggtgcgggttgaactggtttatgattgctgtttttacactttttagacccccagagcgtgcaggtgatggagtgaacgcgcggtgctacagtaatactctaagacaagtttgggcagggaaggaaacacctacaggaggccaggtggccaagatttagacggtttggggagttgtatttgttgccacgtacaaagggacaaaaacacgggcttgtagtggccgcaattccactgctcttgtgcttgtcagctgctactcttcgtctaagaggaccccttcttaatgtgaactcagctgcatgaccgcgcatacttctttcctactgcagcccctgacaggcaatactagcaagtgaagcatccattctacgacagaggtattttcaggacgaaaaaacactcttgagtcgccacaaaaagtgagtgttccgacaacgcgggcggagcagtttggagtttgggagcagtgtattgatctgactgagcttgagagagtgtgtgacattctggagggagaagggaaaggggagatgatatgatggatatgggttggggagatgatggtataggactggttgtatatggaaacgatgcttgatcgtgttattccactcagctgtgtacctccttacgccacaccctacagtcacgttttcaccacccccatctcaaacaacactaacacagtgttctacgaagacaccgccgactccgattggcagtcgtccgactacgacagtgaacatgcgaccgtcaacgtgcccatccggagcaaacggaaaaagaatgctgctaaggagggcaatccagtggatcccatctctgaggatccgcaccaggaatccttttcagatggaagtgaagctgcgggacccagcgctcccccggccgacggatacatcaacaccgcgcccaatggcgtctactcggaggaagatcttcctggaacccgccaggtgcttccaggacctcaggaggtacccgacgtgacctccactccccagatgaaccgccatcccacagcagatgaatctactcctcagggctcacggtggagccgaggcagagagggatttggacatcttggaagtccctcgttctttagacgacacagtgccacagctgaagatatgacccctggaggaggacctcgacggctgttcaagcgaccacagctcaactcttccaacaccacatcgggccagcgttggagagatatcaaagccgggcttcgtcagcggttccgaaagaagaaggacgcgaaattcagcgctgagaaggcatacaatcaagccattctgggccagcttgctgctggttctcctgcggctctcatggttgcttcctccatggttcgcgacgaaaagaacatgaagcgagtgcccattctgctggagcaggtccgtttcgctctcagagatgtgacaaagaaccctcacgagaagaatcgacagtatcgaatcgatctttcgtacggttccggtcacacgatgctcgcatggacaatctacaaggactacaaggactttatgtttctgcacagtcggctgcgaaccatggccttcaacgccaagaaccccttcaactcgtcaaaacagcctcttcagatccccatctttcctacccgaaacaaggtgggcaagaaaaaaaaaggcaagagagacaagtccaaccctggcgatgaagattacacagacacagatggagagtctgagtgtgatcccgcagagcagcaacaaaaccagcacactccaggagggggatcttcgcatggacctaggatggtgaaccagttctttgacaagcgacggaaaaaccgcaacatgatcaacgagcgcattaagggtgaactggaacgatacctgcgtcagctgttcaaactgctgctcttccgaggtgaagctaacaagctgttccagtttttggagctgtccaacatgtccattcgacttgctcctgaggaccatttccacggcaaggctggattcctggtcaaccgtacgtctgctcgaaaggctggatggagagtgtcgcattggcgagtcgacgatatcagacagatggtggctcgtcacacgtcgaagtggttcatggtgcgccactcgtacattgtgtgtgtcgacaacctgtattccacaacacctcttgaggtgttccttctcgattccaagttcaaggtgtcacacaatctcaaaaagaagaacaaggacggcgttctttcgggtggatccaaccccaatacttctggagagtccgatttggaagaacttcagaaaccgggaagtacccatattactctcaaggtggagaacgcatcccgacagcttaagctggttgccacatctgacaagcagctggcccagtggatggagagtatcaatctgatcaaggagcggtccatttgggctcagcagaagcggtttgactcatttgcgcctgtgcgaaccaactgcaaggcccaatggttcgtcgacgctcgagactacttttggacgctgtcgtgcgctctggatatggccaaggaggtcatttacattcacgactggtggctgtctcctgagatatacctacgtcgaccccctgagggtaaccaggagtggcgtctggatcgtgttttgaagcgaaaggccgagcagggagtcaagatttttgttattgtgtaccgaaatgtcggtcagactattcccattgactcgcagtacaccaagttctcgctgctggacctgtctcctaacatttatgtcatgcggtctcccaaccagctcattcagaatacctacttttgggcccatcacgaaaagctgtgtcttattgatcacacgtgtgcctttgtgggtggtattgatctctgttttggtcgttacgatactgcggaacacgttctcgtcgacgatgcgcccacgtttgtgaatgccgacaagaaggacggctacaccggtcgaactcagctcttccctggtaaggattactctaacccccgtaccaaggacttcttctcgttggacaagccttttgaagatatgtacgaccgtcagaaggtgccccgcatgccctggcacgatatccacatgatggtagtaggccaacctgcacgtgatctggtgcgtcatttcgtgcagcgatggaactatgttcttcgacagaagcgtccttcccgattcacgcctcttctgctgcctcctccagacttcaaggaggaggagctggccgagtacaagctgaatggtacgtgtgaggttcagattctgcggtctgcttgttcctggaacactggagtcaaggagcacgagcagagtatccagaatgcgtacatcaagagcattgagcagagtgagcatttcgtctacattgagaaccagttttttatcactcacacctcgtggcacaatattgtgattgaaaacaagatcggagacgctctggtcaaccgaatcatcaaggcccatcagaatgacgaggattggcgtgccatcattgtcattcctctcatgcctggttttgaagccgaggtcgacgagtccgagggttcgtcggtgcgtgtcatcatgcagtgccagtacatgtccatttctcgaggtgccaactgcatttttgcacgtcttgagaacgctggcatccatcccgaagactacatttgcttcttttcactgcgaaagtggggcaagatcggtccccacgacaagctggtcaccgagcagctgtacatccacgccaaggctatggttgtcgatgaccgtattgccattattggttcggcgaacatcaacgaacggtctcaacgaggtactcgagactctgaggtcgcagctattgttcgagacacccatactgtggactctgtcatggctgggcgtccctacaaggtgggccactttgcacacaccctgcgtctccatctgatgcgagagcacatgggcgttgatgtggaccatgtggagttcattgagcgaaaggctgagcttcttgagaaacacaagagagctgaggctacccaagatgagccccaggagcttcctatcatcaagaacaagtccacacgccgagcacgggggcgtggacgacgttcccagcgaggaggagaggattccgatggagcttccggctctgaagatgagcgagttctcgatatctcaacctctatcttcggggagacggttcaggctgccaaggccgctaacggtaatggcaatggcaacggtaacggtgctgatgcgtctgctgcggagtctgtcccttccgtcatggctcccacccgaagatactcttaccaggatggagtacctgcctctccttctattgacgaggactgtgttgtcgaaggcacaactctggacaatgctgaccagcagctagccaacttggacgatgcgtacgattccgacgccgaggtgctactgcgagactatgatgaggatgtggatcagatcaaccggggcttggacatctacaccttcaactctctagccggtgtggacaacaagggcatccgagagaagaagcttgtctcttctgataaccgtatccagggcaacgagaagcaccgtgctgacgttgagggccggggtctggaccgaggcgccaactcgaaggagtctgcggaggacttccgaatccgaatccagcgacacgcccagtttgccaacaagctgacggagcacgagctgcagcagaccagcgagacggtggacgatgtgacggaacttaagaagcgcgtctactacgagcagatggagctcaagcagcggtaccacaattcgcacatggaggccgagaaggcgttcaacaatcccccacagttggaggactgcgagaacgagaagaacggcgttaatggagtcaacggcaacagtggctccactgattccactgcacctgtaaatcttgtctccgatgctaacggcaacattcaccctgaacatcctgagacatctgatcattcttcggactcttttgaagatgagggcttcgagatccccaagatccctcctcccgtagacccctactgttttaaggaccctttggacgacgacttttactacgacatctggctggccacagccgaaaagaacaccaggctcttccgggaagtgtttcgatgccagcctgacgacgaggtgaccacctggagagattacaaggatttcaccacttacgccgagcggttctccatgtctcaggacaccggcgacttgaagtcttcgtatgcagatgaacaccaagctcattcccccttggctgtcaagatcaatggcaatgacaaggaagacgagaagaaacagaacaagcacaaggccacacatgactacgacaagtattatgcccgtcaccagcaccaccgatcgtcttcgcctcactaccgaaggtcccatgtgcctctcaagaccggtgccaacttcactgatcgacttgcacacggtgttgatgatgccgctggtcatctcaagtacgccctcggacgggccaaccgccgaggcagccaccatgacactgttgatgtggatgattctcctcccagggacttccttcctgacgatttcaacgacccgatccctgtcgaggaggtggacaacaccaacctcaacaacaaccagcagctcaaccaggagctttacgagtcgcaggtggagggcgagaaccctcgatttgtgcctagccacgccattgatggccagcaccctcgaaagcgtgagaagcgacacattggtgaggttgatcagcctgccgagaagccccccatcaacgaggcttacagcactggggtctcaggccggcttgggcagcaccagcagccccatcttaagcaggaagttgatactgattttaaggatacctcttctgctgctcatacgccattccaccatacccccaacgccaggcgaagacgacgacgacgggctcgacagagatctggcatggacaaggtgcacgacaaggcaacggccgagaagattctcaacggtatccaggggcatctggtcatgttccccacccactggctgtacaaggaactcgacagcggcaactggttctacaacctggatcgaatccccccgatcgaaatttatgattagaatggtaatatattggtatgataaataatgcaagcacttgcatgagtgtactgtacgtaagcaagtagtgacagttcagttaatcaaatgcaaacccgctgcgacaaacacgtgctaagctcttcagattcttcatcactggtctcgaaatcaaagagctcacaggtgtcttcttcctctttctggtcactggcaaagttccagtattgacgacggacgattcccgaaagcgtccagatttccacctgttgtttgatcttgtcgaagaagattagtgcaatgatgaccgaggccagctcgggaatgatgtacgggagtcgagacatgatctcgtctggttttaggggagtcttgtcgtcttcggggagatctgtgaaggtggtgtacagaaacgtgaatgcaatatggatctgaagaatcttttgtgtttccagagcattgtgaacgattttcacgatggtggcgtcgtaggcagccttcttatgagcgtcaggaggtccattcagcccggtggagaaagagtccacgatcacgtatctgattttcgagctgttgtttgacttttcaatgtgtccgatgattgtgtctgattgcagctcggtggcgagctggtcgaaggtggttttcacaatcacgaggttctggcgaatataactggcaagcacattttcacccagaccagtggattgatccagcacaaagtagttgatgacgttgtttttcgtccatgagctgtcgagatccagaaccagaaccgtcttgttctcgcctcctggagtgtcgaatggcaaacacacacggcacagctcgtacagcgccgtggttttgcctgatcttggagctccggtcagagtgatcagccctgcaagctgtgctgatggcaaatgcagccagtgtaacaagttttctgccgcatcagacggaccgtaatgctgaatggcgtcgaacaagttttccttcttgttttccgtttccatgatggtagttgtcagtgatgttc

SEQ ID NO: 73 - protein, 1829 aa, Yarrowia lipolytica
METMLDRVIPLSCVPPYATPYSHVFTTPISNNTNTVFYEDTADSDWQSSDYDSEHATVNVPIRSKRKKNAAKEGNPVDPISEDPHQESFSDGSEAAGPSAPPADGYINTAPNGVYSEEDLPGTRQVLPGPQEVPDVTSTPQMNRHPTADESTPQGSRWSRGREGFGHLGSPSFFRRHSATAEDMTPGGGPRRLFKRPQLNSSNTTSGQRWRDIKAGLRQRFRKKKDAKFSAEKAYNQAILGQLAAGSPAALMVASSMVRDEKNMKRVPILLEQVRFALRDVTKNPHEKNRQYRIDLSYGSGHTMLAWTIYKDYKDFMFLHSRLRTMAFNAKNPFNSSKQPLQIPIFPTRNKVGKKKKGKRDKSNPGDEDYTDTDGESECDPAEQQQNQHTPGGGSSHGPRMVNQFFDKRRKNRNMINERIKGELERYLRQLFKLLLFRGEANKLFQFLELSNMSIRLAPEDHFHGKAGFLVNRTSARKAGWRVSHWRVDDIRQMVARHTSKWFMVRHSYIVCVDNLYSTTPLEVFLLDSKFKVSHNLKKKNKDGVLSGGSNPNTSGESDLEELQKPGSTHITLKVENASRQLKLVATSDKQLAQWMESINLIKERSIWAQQKRFDSFAPVRTNCKAQWFVDARDYFWTLSCALDMAKEVIYIHDWWLSPEIYLRRPPEGNQEWRLDRVLKRKAEQGVKIFVIVYRNVGQTIPIDSQYTKFSLLDLSPNIYVMRSPNQLIQNTYFWAHHEKLCLIDHTCAFVGGIDLCFGRYDTAEHVLVDDAPTFVNADKKDGYTGRTQLFPGKDYSNPRTKDFFSLDKPFEDMYDRQKVPRMPWHDIHMMVVGQPARDLVRHFVQRWNYVLRQKRPSRFTPLLLPPPDFKEEELAEYKLNGTCEVQILRSACSWNTGVKEHEQSIQNAYIKSIEQSEHFVYIENQFFITHTSWHNIVIENKIGDALVNRIIKAHQNDEDWRAIIVIPLMPGFEAEVDESEGSSVRVIMQCQYMSISRGANCIFARLENAGIHPEDYICFFSLRKWGKIGPHDKLVTEQLYIHAKAMVVDDRIAIIGSANINERSQRGTRDSEVAAIVRDTHTVDSVMAGRPYKVGHFAHTLRLHLMREHMGVDVDHVEFIERKAELLEKHKRAEATQDEPQELPIIKNKSTRRARGRGRRSQRGGEDSDGASGSEDERVLDISTSIFGETVQAAKAANGNGNGNGNGADASAAESVPSVMAPTRRYSYQDGVPASPSIDEDCVVEGTTLDNADQQLANLDDAYDSDAEVLLRDYDEDVDQINRGLDIYTFNSLAGVDNKGIREKKLVSSDNRIQGNEKHRADVEGRGLDRGANSKESAEDFRIRIQRHAQFANKLTEHELQQTSETVDDVTELKKRVYYEQMELKQRYHNSHMEAEKAFNNPPQLEDCENEKNGVNGVNGNSGSTDSTAPVNLVSDANGNIHPEHPETSDHSSDSFEDEGFEIPKIPPPVDPYCFKDPLDDDFYYDIWLATAEKNTRLFREVFRCQPDDEVTTWRDYKDFTTYAERFSMSQDTGDLKSSYADEHQAHSPLAVKINGNDKEDEKKQNKHKATHDYDKYYARHQHHRSSSPHYRRSHVPLKTGANFTDRLAHGVDDAAGHLKYALGRANRRGSHHDTVDVDDSPPRDFLPDDFNDPIPVEEVDNTNLNNNQQLNQELYESQVEGENPRFVPSHAIDGQHPRKREKRHIGEVDQPAEKPPINEAYSTGVSGRLGQHQQPHLKQEVDTDFKDTSSAAHTPFHHTPNARRRRRRRARQRSGMDKVHDKATAEKILNGIQGHLVMFPTHWLYKELDSGNWFYNLDRIPPIEIYD

SEQ ID NO: 74 - DNA, 3863 bp, Yarrowia lipolytica
aggtgcccattcagattcagcccgggtaccagcccttgaagggtctttacagtgacgtgagtatgataacagaaggaaacgacgaccggtgaggaaagtgatggtatttgtggtgggagccatttgtcattggcggttggtcattggtcattggtcattggtcattggtgattagcgattggtgattagcgattggtgattggtgattggtatggtctgattactgaatggacttgagacacgaagcagatggaccatgataaaccatgatacagctcttgacttacttgatttgaacgtgtacttgagaaacagttttattgttttcgagctattcaactgcttggtctgagtctgtttggtgtgtgagctcacaccgtttggtatgtttggtatcactatgtcaatgcttctgccaatgtcatgtagtctccagaatgaggcacttgttttccttctctgtcgtttgtggcacctccttatgtgtctcggttcttgtggcacgtttttttcttttcgttctatctcggttccctcgtcatctcatgatactcaccgttgtcaggctggcagctcgcacttgcaccatgtttcggtctttgctttcccggctacgttccctgcccacacatatatctgtttctacgtgcatgtgattcccccacatgtgcctgtgatatctatgtaccgtggctaggattatgcctgtgattgtgtcatgataaccattctgcgcagtgtgattgacgatcagggtcttggtgggtacaaactagctttttgggtttctttttttcgctccaaaatgtgagcgtgaaaatgcacgtgagctaaaagagtcccaacacccaccaactgcgctggctcgactgttgagcatgttgagtgagaggagaggagaaacaaggcgcggaaaaaagggacgagaaaagaaccatagggccaaagacaaaaaaccacagacacatgtcacatgcccggattatgccccgatcagtgcatgcggaagaccgacccggcgatagaaggagaagaaatacgaggtgcagaagtgtcatcctccgccgtgattgcattttgcgtggtaataaccacgagccacgcactttgtccacgcacatgtctatggtgtaattgtacctttcctcccaacgcacccaacccaaacctaaaccctaagttgcacaaaacacatgaacgtgatactgcaaacttcccctctgataaaggcgctgtgtccatatcatctcagcacaaccacatctccaacaccaacacgacttttgccacctcgggagcccagagccagtaccaccggctctccaacaacaccaagaccttcactcgagactccccctaccattcgcgaaccccttcagtggatgacgacggaataagacaagcatcgtttctcaaccgagtctaccagtcctccaagtcctacagtccccgattccgctacggagcagagatagtcgaacggtgggcaagttctgaaaacaccaacgcagcaacaccttcggcgtcaaccacaaacagcaacaacagcaaccacagcaacacgcctgtttcaaccacaccctcaagcacaaccatgcccaaggctctgtcttcaaagtcgctggacgccgccagcatccacatggcatcgaacggtgcgcctccactgatacaaaagtcgtcgttgcacgactttgagcgccttaaatcgggaattgacgacattcaaaactcagacaccaacggtacccagtacagtgtcgatgtcggctcccagggcctgcgcatgcgtatccagacccaagggtacgcccccagcggcaacagcaacagaggcagccccgtgccaccgtcaccggcgctgtccacgtcgacctcctcgggctccacttctgctgcagcctccagatccacctcgtctcctgtgccacaacaacctcagacagctgccacagctgcaggaggacctcgacccggacaacctcggtcggcatggcaggaggtgctgatcagcgccacgtcgctggcatcattgtcgcaggactcgcgaaagcgactgcgatactgtctgcatctccttaagctcgccaacgcccatctggcttccaccgtcacaaagctgcaaggcgccattgccgaggagactgcatacagtctcgcgcagtccattgccgccaaccatgtgcctcacaacgagcgacaggcgtacctgcatcagcctccctccgctgagcctgctctctccatcaccgctctcaaggccgatgtcgtgtccacgatccgcaagatcatcaaggttgtcagccagtacgccggcaacagtctacccgagccggctcggtcgcacatccggacatacattcttggcctgccctctcggtgggcctctaccaccgcttccactaacatcaccccaacccggtctcctgccggttcccagtctcctgtgggctctcctaaggagtgtgtcacacctgaccaccagggtcctcctcttcccacaaacacagtgtcgtctccagtgcccgagggcccttctgacgagcagttgtcgcggcatcgaatcgaggttgaggctggaggcaaggtgttgattcttgccaacgaggctctcgatatgctgggcaacatcatcagtattgtggatggcaccctggagcgtgccgagggttggtgcgagggcatcaaccgggtcaagcagagagttgggctcggtgaaaccgctcctggcgcagctggggaacctggagcctccaccgaggcttctgctgaggcttctgctgaggcttctgccagtgccgccgctgcgtactctgctgttgatacctccacggaccaggacgttgagatgggagatgcctaaggctggcatgacacgacaagggacacagatgaaatgcatgatggatgatgacacaacacacacagcatgaagcatggcatgatggagaacgacatacagcacaacacaacagagagaggatagcataggcacaacatatttcatgtactcaccacacacagaacaagcgacactgacacatacacacacacacacacacacgcacacacagcagcagcagcagcagcggcagcagcagtacagcattatgcaacccctcacagatgatgcaacacttaaatggttacacacatggcatatttaatcacctctcttgtgggtgctccagaagatggcttttggcgtcggatgacggcactggttttcattttcctcgattacatgtctgtctcgggcgttgtttgtcccctcggcactttttcggcctcggctacccggtcctcgggaaagtgtgtcaaggttcacaacggtaaaaaaagtcaaaggtggtggcgttttgggaggttcggtttgaagagcggagctactgtgggcagcagcgttaaacttggtgacaagacaacggctggacgctgacgtttaccattaattggcgctgctgccgtgggtggctactggatttggtcttttgcacgaagacttatgcgttgctgcttgcgagtgtgtctctgtcttggggtggtttgccacgtgacgggccggtgtgacgccgagcggaattcgtgtttcttgccccttttatttccgacatgttccggctgctgcttctccagcatgttaccccacaaccgacgggtaaaatttctggacgcgcacaaaccttttttggcttgcggtccaggggaaaggaaccgacagatgtctaccaccaccatcaacaagagcacaccaccagtaccaggagtacactgttaccgccattagcaagtagagtttatttatataatgcaaagattatgatgggttttgggccgtggtgttggtgttggtgtctgtagaa

SEQ ID NO: 75 - protein, 620 aa, Yarrowia lipolytica
MRKTDPAIEGEEIRGAEVSSSAVIAFCVVITTSHALCPRTCLWCNCTFPPNAPNPNLNPKLHKTHERDTANFPSDKGAVSISSQHNHISNTNTTFATSGAQSQYHRLSNNTKTFTRDSPYHSRTPSVDDDGIRQASFLNRVYQSSKSYSPRFRYGAEIVERWASSENTNAATPSASTTNSNNSNHSNTPVSTTPSSTTMPKALSSKSLDAASIHMASNGAPPLIQKSSLHDFERLKSGIDDIQNSDTNGTQYSVDVGSQGLRMRIQTQGYAPSGNSNRGSPVPPSPALSTSTSSGSTSAAASRSTSSPVPQQPQTAATAAGGPRPGQPRSAWQEVLISATSLASLSQDSRKRLRYCLHLLKLANAHLASTVTKLQGAIAEETAYSLAQSIAANHVPHNERQAYLHQPPSAEPALSITALKADVVSTIRKIIKVVSQYAGNSLPEPARSHIRTYILGLPSRWASTTASTNITPTRSPAGSQSPVGSPKECVTPDHQGPPLPTNTVSSPVPEGPSDEQLSRHRIEVEAGGKVLILANEALDMLGNIISIVDGTLERAEGWCEGINRVKQRVGLGETAPGAAGEPGASTEASAEASAEASASAAAAYSAVDTSTDQDVEMGDA

SEQ ID NO: 76 - DNA, 4247 bp, Saccharomyces cerevisiae
tcactcaaccacctccaaaaaataacaggttcatctaaagtaaaagactttaacttgctcttagtttccaaattaaatatctgcacgatagtaccatttgctctaacggaaataaccatctgagatggatgcatgatagcagaatcaccgcccatattcttccttgtcacttcattgcctttggccaaatccacgattgcaacagagtttgtaccgtcctttgtttctctaacagtgacgaagtggtcactctcgaaagtagttgatctgaagtcaaggaattgaggggaaattcctaaggacatcagatcgaccaattcggtaaattcaatgggtaggtcactcattggttagaactttcgtgataatttatttttatagttgaatatcttctttctctctcaactctgatccggattgtcgaggtttcaataagttactctgaacaactaatcaaaatatctccttatttctgtagattccttcagttccactttttacttttcttaattctctttgtatttattcctagcgacgaaaaatgcgagatctcgaccaaaaaaagggggtagggtaataaaattaaccctattattttttaactttaaaacctataatgtgctaatattttattataaacctcctttttttgcgttcaaaccctgacacattttaagccctatatttacggtattagttgattaaactccgaagcgaaaggaattcggtcattagcggctaatagccgttggggtaaatcacctacaagcaagtacacaagagaacgttggcgttgttaagtcaaagcactaatacattggggctttaagagtgtttataaaggtctaacctgtaaaaattatttaaacaacttgaacaggccttaaagttttcctcattccgctcatcatcactaatattgctctccgtttttgaatacacacttgacactaataagtatcacagaaaaaaagaaaatataataaattagtattgcgatatgacgagacgtactactattaatcccgattcggtggttctgaatcctcaaaaatttatccagaaagaaagggcggattcgaaaatcaaagttgaccaagttaacacatttttagagtcatccccggagaggagaactctgacgcacgccttaatagaccaaatagtgaatgatcctatattgaaaactgatacggactattacgatgctaaaaaaatgcaagagagagaaattactgccaaaaaaatagctaggcttgctagttatatggagcacgatatcaaaacagtgcgcaaacactttcgcgacactgacctgatgaaagagttgcaagcaaatgatccagacaaagcttcgcctttaacaaacaaagacctttttatattcgataagagattgtcacttgtagcaaatattgatcctcaattgggtacgcgcgtgggtgtacacttggggctatttggtaattgtatcaagggcaatggtactgatgagcaaatccggtattggttgcaggagagaggtgccactttgatgaaaggtatatatggctgttttgcaatgactgagttaggacatggttccaatgttgcccagctgcagactagggctgtgtacgataagcaaaatgatacttttgtaattgatacacctgatctaactgccaccaaatggtggattggtggggctgcccattctgccacgcacgctgccgtgtacgccagattgatcgttgaaggtaaagactacggtgtaaaaacattcgttgttcctctgagagacccttcgactttccaactgttagctggtgtttccataggggatattggagcgaagatgggtcgtgacggtattgataatggctggatccagttcagaaacgtagttatccctagagaatttatgctaagtagatttaccaaagttgtccgttctccagatggttcagtcaccgtcaaaactgagccacaattggatcaaatttctggttatagtgcattgttaagtggtagagttaacatggtcatggattcatttaggtttggctccaaatttgctactattgctgtacgttacgcggttggtcgtcagcaattcgcacctagaaagggattgtctgaaacacaattaatcgactatccccttcaccaatatcgtgttttaccacaattgtgtgttccatatttggtgtcacctgtagcttttaagttaatggacaactattattccactttggacgagttatacaacgcttcctcatctgcatacaaagctgctctggttaccgtgagtaaaaagttgaagaatttatttattgatagcgccagcttgaaagccaccaatacttggttaattgctacactgattgatgagttgagacagacttgcggaggacatgggtattcacagtataacggatttggtaaaggctatgacgactgggtggttcagtgcacatgggagggtgataataatgttttatctttaacttcagcaaaatcaatattgaaaaaatttatcgattcagccacaaagggtagatttgacaacacactggatgtggactcattctcttacttaaaacctcagtacataggatctgtggtttctggagaaataaagagtggtttaaaggagttgggtgattatactgaaatttggtctatcaccttaatcaaattactggcacatattggtactttagttgaaaaatcaagaagtattgatagcgtttctaagcttttagtcttagtatccaaatttcatgccttgcgctgcatgttgaaaacctattacgacaagttaaactctcgtgattcacatatttccgatgaaattacaaaggaatctatgtggaatgtttataagttattttccttgtattttattgacaagcattccggagaattccaacaattcaagatcttcactcctgatcagatctctaaagttgtgcagccacaactattggctcttttgccaattgtgaggaaagactgtataggtctgacagactcctttgaattacctgacgcgatgttaaattctcctataggttactttgatggcgatatctatcacaattacttcaatgaagtttgccgcaataatccagtggaggcagatggggcagggaagccttcttatcatgcgctgttgagcagcatgctcggtagaggtttcgaatttgaccaaaagttaggtggtgcagctaatgcggaaattttatcgaaaataaacaagtgagtagaggtttcctgttttccttcgaaccctctgttttgcgacttttgtttcaattcaactagtgtcgccaagttttaacaaaaagttacaaaatcctagtgagaggccatcttatgtgcataacggtactctctatctatttacatatctaatactattcacataactatgacgaatcaatgacatgactacatttaccaatgtatagtagtaaaaaaattgccatctcctagaatcgaaccagggtttcatcggccacaacgatgtgtactaaccactatactaagatggcaggtaattattgagtgtttactcaatatctcagaattatatacataacaaataataaatgtgctgtgttggcacttgttggaatgaagctctaaaacatcatgatgattgtgtggattctgatgtaattgttgggattccatttttaataaggcaatatgttggaacgagagtaattaatagtgacatgagttgctatggtaacaatctaatgcttacatcgtatattaatgtacacatcgtatacgtttaagtgtgattgcgcctattgcagaaggaatgttaaacgagaagctcagacaatactgaagctgtgttaaagacctattagctgaacatgatatggtaggtacatatatgaggaatatgagtcgtcacatcaatgtatagtaactaccggaatcactattatattggtcatgattaatatgaccaatcggcgtgtgttttatatacctctcttatttagtataagaagatcagtactcacttcttcattaatactaatttttaacctctaattatcaacacaataatattaggtatgtagatatactagaagttctcctccaggatttaggaatccataaaagggagtctgcaattctacacaattctataaatattatcatcatcgttttatatgttaatattcattgatcctattacattatcaatccttgcgtttcagcttccactaatttagatg

SEQ ID NO: 77 - protein, 748 aa, Saccharomyces cerevisiae
MTRRTTINPDSVVLNPQKFIQKERADSKIKVDQVNTFLESSPERRTLTHALIDQIVNDPILKTDTDYYDAKKMQEREITAKKIARLASYMEHDIKTVRKHFRDTDLMKELQANDPDKASPLTNKDLFIFDKRLSLVANIDPQLGTRVGVHLGLFGNCIKGNGTDEQIRYWLQERGATLMKGIYGCFAMTELGHGSNVAQLQTRAVYDKQNDTFVIDTPDLTATKWWIGGAAHSATHAAVYARLIVEGKDYGVKTFVVPLRDPSTFQLLAGVSIGDIGAKMGRDGIDNGWIQFRNVVIPREFMLSRFTKVVRSPDGSVTVKTEPQLDQISGYSALLSGRVNMVMDSFRFGSKFATIAVRYAVGRQQFAPRKGLSETQLIDYPLHQYRVLPQLCVPYLVSPVAFKLMDNYYSTLDELYNASSSAYKAALVTVSKKLKNLFIDSASLKATNTWLIATLIDELRQTCGGHGYSQYNGFGKGYDDWVVQCTWEGDNNVLSLTSAKSILKKFIDSATKGRFDNTLDVDSFSYLKPQYIGSVVSGEIKSGLKELGDYTEIWSITLIKLLAHIGTLVEKSRSIDSVSKLLVLVSKFHALRCMLKTYYDKLNSRDSHISDEITKESMWNVYKLFSLYFIDKHSGEFQQFKIFTPDQISKVVQPQLLALLPIVRKDCIGLTDSFELPDAMLNSPIGYFDGDIYHNYFNEVCRNNPVEADGAGKPSYHALLSSMLGRGFEFDQKLGGAANAEILSKINK

SEQ ID NO: 78 - DNA, 973 bp, Yarrowia lipolytica
aaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctcc

SEQ ID NO: 79 - DNA, 927 bp, Artificial Sequence
  Nucleotide sequence of a modified Y. lipolytica FBAIN gene promoter
aaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacagaattccgagccgtgagtatccacgacaagatcagtgtcgagacgacgcgttttgtgtaatgacacaatccgaaagtcgctagcaacacacactctctacacaaactaacccagctctcacacaatg

SEQ ID NO: 80 - DNA, 971 bp, Yarrowia lipolytica
gacgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgccatggaaccttgcggatatcgatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacatg

SEQ ID NO: 81 - protein, 535 aa, Macadamia tetraphylla
MSISNSPENLDQKPNIRNESEDRSFSVRRRTNSNVVNAVPDSCSKVGTIETVTKFSSVDSAAEMSSGEDRNRIGNGDDRKEEANSLSNGQVYNERVGIGEGRSPEAEVPFRFSYRASAPAHRRVKESPLSSDAIFRQSHAGLFNLCILVLVAVNGRLIIENLMKYGLLIRAGFWFSSKSLRDWPLLMCCLSLPIFPLGAFTVEKSAKHKFIPEPVVMSLHVLICTASLLYPIFVILRCGSAVLFGIALMLLASIVWLKLVSYAHTNCDMRALAKLTDKVASKSSDMDYSYDVSFESLAYFMVAPTLCYQASYPRSTCIRKGWVIRQLVKLVIFTGVMGFIVEQYINPIVQHSQHPLKGNLLYAIERVLKLSVPTLYVWLCMFYCFFHLWLNILAELLRFGDREFYKDWWNAKTVEEYWRMWNMPVHKWMVRHIYFPCLRNGLPREVTLVIVFFLSAIFHELCIGVPCHIFKFWAFIGIMFQVPLVLITNYLQNKFRSSMVGNMVFWFFFSILGQPMSVLLYYHDVMNRKENGVSK

SEQ ID NO: 82 - DNA, 1634 bp, Macadamia tetraphylla
ggtctctccaccacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtaggtatagagacc

SEQ ID NO: 83 - protein, 352 aa, Garcinia mangostana
MLKLSSSRSPLARIPTRPRPNSIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTR

SEQ ID NO: 84 - DNA, 1081 bp, Garcinia mangostana
ggtctctaatgatgctgaaattatcttcttcacgttcgccgttggcaagaattccgacccgacctcgacctaattccatcccgcccagaatcattgttgttagcagttcttctagcaaggttaatcctttgaaaactgaagctgttgtcagctcgggattggcggatagattgcgactgggttcgttgaccgaagatggtttatcctacaaagagaaatttattgtgcgatgctatgaggtgggcatcaataaaacagccaccgtggagactattgcaaacttgctccaagaagtcggttgtaaccacgcccaatccgtgggctattccactggcggcttttctactaccccgaccatgcgaaaattgcggctgatttgggtcactgccagaatgcatatcgagatatataaatatcccgcctggtctgatgtcgttgagattgaatcgtggggacaaggtgaaggcaagattggtacacgccgagactggatcttacgtgattatgctaccggtcaagttatcggacgagcgacatcgaagtgggttatgatgaatcaagatacccgacgacttcaaaaggtcgatgtggatgtgcgggatgagtacctcgtgcattgtccgcgtgagttgcgtttggcgttccccgaggagaataattctagcttgaagaagatctccaagttggaagatccttcccaatattcgaaactgggcttggtgccacgacgtgccgacttggatatgaaccaacacgtcaataatgttacttatattggatgggtcctggaatctatgcctcaagagatcatagacactcatgaattgcagacaattaccttggattatcgacgcgagtgccaacatgatgatgttgtggatagcttgacctctcccgaaccctctgaggatgccgaagcagtgttcaatcataacggaacaaacggctccgctaatgtctctgctaatgaccatggttgcagaaatttcttgcatctgttgcgattaagcggcaatggtttggaaattaatcggggacgaaccgaatggagaaagaaacccacacggtagtctaagagacc

SEQ ID NO: 85 - protein, 355 aa, Garcinia mangostana
MFKISSSLSPVDQIPPISPLPRPRPRPITPRVLAVSSSSGKIVNNPLKAETTEAVSGELARRFRLGRLAEDGFSYKEKFIVRCYEVGINKTATVETLANLLQEVGGNHAQSVGFSTDGFATTHSMRKMHLIWVTARMHIEIYKYPAWSDVIEVETWIGAEGRIGTRRNWIIKDCATDEVIGRATSKWVMMNQDTRRLEKVSDDVREEHLVFSPREPRLPFPDENNSSLKKISKLDDPAQYSKLSLEPRRGDLDMNQHVNNVTYIGWVLESMPQEIIDTHELQTITLDYRRECQHDDLVDSLTSPEPSEFSETTNGSANVSPNDNRCLNFLHLLRLSSDGSEINRGRTVWRKKPAK

SEQ ID NO: 86 - DNA, 1090 bp, Garcinia mangostana
ggtctctaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctaagagacc

SEQ ID NO: 87 - protein, 545 aa, Pseudomonas chlororaphis
MRDYEHVVESFDYLQSATQDLHGELTALNACVECCDRHAHGEAVALYCEAQDGHAERYRFRDLQRQAARFGNFLREQGVKPGDRVAGLMPRTVELLIAILGTWRIGAVYQPLFTAFGPKAIEQRLNCSNARWIVTDPHNRPKLDDVTDCPSIVVTGGAPQNPADHHFWSALNRQADDCAPVLLDASAPFLLMCTSGTTGPAKPLEVPLSAILAFKGYMRDAIDLRADDRFWNLADPGWAYGLYYAVTGPLACGYATLFYDGPFTVESTRHIIAKYAINNLAGSPTAYRFLIAAGAEFADAVRGRLRAVSSAGEPLNPQVVRWFAEQLGVVIHDHYGQTEIGMVLCNHHGLRHPVREGSAGYAVPGYRIVVLDKAHRELPAGQPGVLAVDRERSPLCWFDGYLGMPTQAFAGRYYLSGDIVELNDDGSISFVGRNDDLITTSGYRVGPFDVESALIEHPAVVEAAVIGKPDPQRTELIKAFVVLNTPYLPSPELAEELRLHVRQRLAAHAYPREMEFVDHLPKTPSGKLQRFILRNQEIAKQQALG

SEQ ID NO: 88 - DNA, 1660 bp, Pseudomonas chlororaphis
ggtctctacaaatgcgagactacgagcatgttgtggaaagttttgactacttacaatccgcaactcaagaccttcacggagagttgaccgccctgaatgcttgtgttgagtgttgtgatagacacgcgcatggtgaagctgttgccctttactgtgaggcccaagacggtcacgcggaacgatatcggttccgagatttgcagcgtcaagccgcacgatttggtaactttcttcgagagcagggagtgaaacctggtgaccgagtggctggtttgatgccccgtaccgtcgagttgcttatcgctatcctgggaacatggcggattggagctgtgtatcagcccttgttcaccgcgtttggtccgaaagctattgagcagcggctgaattgtagcaatgctcgatggattgtcactgatccccataatcgaccaaaattggatgatgttaccgattgtccgagtatagtcgtcactggcggtgcaccccaaaatccggcagaccatcacttttggtccgccttgaatcgacaagccgatgactgtgctcctgtcttattagacgcttccgctcccttcttgctcatgtgcacttcgggtactaccggccctgcgaagcctttggaagtccccctctcggccatcttagcttttaaaggttacatgcgagatgccatcgatcttagagcagatgatcgattttggaatttggccgatccaggatgggcgtacggactgtactatgccgtcactggacccttggcctgcggctatgccaccttgttttatgatggaccgtttaccgttgagtccactcgacatataatcgctaaatatgccataaataatttggctggatcaccgacagcttatcgattcttgattgctgccggcgctgaatttgctgatgcagtgcgcggtcgattgcgtgctgtgtctagtgcgggcgagcccttgaatccccaggtggttcgatggttcgctgagcaactgggagtcgtcatccatgatcattatggtcagacagagataggtatggtcctttgtaaccatcatggacttcgacatcccgtgcgtgagggttcggcaggatacgcagttcctggttatcgtattgtggtcctcgacaaagctcatcgagagctgccggcaggtcaacctggagttttggcagtcgaccgagaacgatcccctctgtgttggtttgatggttatttgggaatgcctacacaagcgttcgccggacgatactatttgtccggagacatcgtcgaactgaacgatgatggttctatttcttttgttggtcgaaacgacgatttgatcactacctctggatatcgagtgggtccatttgacgtcgagtccgccttgattgagcatcccgctgtcgttgaagctgcagttataggaaagccagaccctcagcggacagaactgattaaagccttcgttgtcttgaatactccatatctcccttcgccagaattagccgaagagttgcgtttgcacgtgcgacaacggttggctgcccacgcatacccccgagagatggaattcgttgatcacttaccgaaaacaccttcgggaaaactgcagcgatttatcttgagaaatcaagaaatcgcgaaacagcaagccttgggatagtctaagagacc

SEQ ID NO: 89 - DNA, 1660 bp, Pseudomonas chlororaphis
ggtctctacaaatgcgtgactacgaacatgttgtggagtcttttgattacttgcaatctgcaacacaagatctccacggtgagttgaccgcactgaatgcttgtgtggagtgttgtgatcgacacgcgcatggagaagctgttgccctctactgtgaggcccaagacggacacgcggaacgttatagattccgagatctccagcgacaagctgctcgttttggtaactttctcagagagcagggtgttaaacccggcgaccgagttgcaggattaatgccccgaaccgtggagttgcttatcgctatcctgggaacttggagaattggagccgtttatcagcctttattcacagcttttggaccaaaagctattgagcagcgactgaattgttctaatgctcgatggattgtcacagatcctcataatcgacctaaattggatgatgttacagattgtccttcgattgtcgtcacaggcggagctcctcaaaatccagccgaccatcatttctggtcagctctcaatcgacaagccgatgactgtgcccctgttctcttggacgcttcggctccctttctgctgatgtgcacttccggtacaaccggtcccgctaagcccttagaagtccctttgtctgccatattagcatttaaaggatacatgcgcgacgctatagatctgcgagccgatgatcgattttggaatttagccgatcccggctgggcgtacggcttatactatgctgtcactggacccttggcatgcggctatgctacacttttctatgatggtccgtttacagtcgagagcacacgccatattatcgcgaaatatgctataaataatttggctggaagcccaactgcttatcgatttctcattgctgccggtgctgaatttgctgatgctgtccggggccgtttgcgagctgtgtcctctgctggcgagcctttgaatcctcaggtggtcagatggttcgctgagcaattgggagttgtcatacatgatcattatggtcagacagagattggtatggttttgtgtaaccatcatggactgcgacatcccgtgcgtgagggttctgccggatacgctgtccctggttatagaattgtggtcctcgacaaagcccatcgtgagctccccgctggtcaacccggagtgttggcagtggaccgtgaacggtcccccttatgttggtttgatggatatttgggtatgccaactcaagctttcgctggacgttactatttgtctggagacatcgtcgaattgaacgatgatggttctatttcgtttgttggacggaacgacgatctcatcacaacctcaggatatagagtgggcccatttgacgtcgagtccgccttaatagagcatccggctgtcgtggaagccgctgtcattggaaagccagacccccagagaactgaactgatcaaagccttcgtggtcttgaataccccttatttaccttctccggaattagccgaagagttgcgattgcacgtgagacaacggttagcggctcacgcatacccccgcgagatggaattcgtggatcacttgccaaagacacccagcggtaaactgcagagatttatcttgcggaatcaagaaatcgcaaaacagcaagctttaggctagtctaagagacc

SEQ ID NO: 90 - protein, 314 aa, Mortierella alpina
MSIGSSNPVLLAAIPFVYLFVLPRVLAFLPQKAQFLAKCIVVLIATLIMSVAGCFISIVCALLDKRYVINYVVSRLFSFLAARPCGVTYKIVGEEHLDKYPAIVVCNHQSSMDMMVLGRVFPKHCVVMAKKELLYFPFLGMFMKLSNAIFIDRKNHKKAIESTTQAVADMKKHNSGIWIFPEGTRSRLDKADLLPFKKGAFHLAIQAQLPILPIISQGYSHIYDSSKRYFPGGELEIRVLEPIPTTGLTTDDVNDLMDKTRNLMLKHLKEMDSQYSSSTAENGSTHIDADIAKSTATSIGNTDDAITKRRTPKE

SEQ ID NO: 91 - DNA, 945 bp, Mortierella alpina
atgtctatcggatcttctaaccccgtgctgctggccgctattcccttcgtgtacctgttcgtgctgccccgagtgctggccttcctgcctcagaaggcccagttcctggccaagtgcatcgtggtgctgatcgctaccctgatcatgtctgtggccggctgcttcatctctatcgtgtgcgccctgctggacaagcgatacgtgatcaactacgtggtgtctcgactgttctctttcctggccgctcgaccctgcggcgtgacctacaagatcgtcggcgaggaacacctggacaagtaccccgccattgtggtgtgcaaccaccagtcctctatggacatgatggtgctgggccgagtgttccccaagcactgcgtggtgatggccaagaaggaactgctgtacttccccttcctgggcatgttcatgaagctgtctaacgccatcttcatcgaccgaaagaaccacaagaaggccatcgagtctaccactcaggccgtggccgacatgaagaagcacaactctggcatctggatcttccccgagggcacccgatctcgactggacaaggccgacctgctgcctttcaagaagggcgccttccacctggccatccaggctcagctgcccattctgcccatcatctctcagggctactctcacatctacgactcttctaagcgatacttccccggtggcgagctggaaatccgagtgctcgagcccattcctaccaccggcctgaccaccgacgacgtgaacgacctgatggacaagacccgaaacctgatgctgaagcacctgaaggaaatggactctcagtactcttcgtctaccgccgagaacggctctacccacatcgacgccgacattgccaagtccaccgccacctctatcggcaacactgacgacgccattaccaagcgacgaacccctaaggaatag

SEQ ID NO: 92 - DNA, 9103 bp, Artificial Sequence
  Nucleotide sequence of the NotI DNA fragment of pAT207 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgctgaaattatcttcttcacgttcgccgttggcaagaattccgacccgacctcgacctaattccatcccgcccagaatcattgttgttagcagttcttctagcaaggttaatcctttgaaaactgaagctgttgtcagctcgggattggcggatagattgcgactgggttcgttgaccgaagatggtttatcctacaaagagaaatttattgtgcgatgctatgaggtgggcatcaataaaacagccaccgtggagactattgcaaacttgctccaagaagtcggttgtaaccacgcccaatccgtgggctattccactggcggcttttctactaccccgaccatgcgaaaattgcggctgatttgggtcactgccagaatgcatatcgagatatataaatatcccgcctggtctgatgtcgttgagattgaatcgtggggacaaggtgaaggcaagattggtacacgccgagactggatcttacgtgattatgctaccggtcaagttatcggacgagcgacatcgaagtgggttatgatgaatcaagatacccgacgacttcaaaaggtcgatgtggatgtgcgggatgagtacctcgtgcattgtccgcgtgagttgcgtttggcgttccccgaggagaataattctagcttgaagaagatctccaagttggaagatccttcccaatattcgaaactgggcttggtgccacgacgtgccgacttggatatgaaccaacacgtcaataatgttacttatattggatgggtcctggaatctatgcctcaagagatcatagacactcatgaattgcagacaattaccttggattatcgacgcgagtgccaacatgatgatgttgtggatagcttgacctctcccgaaccctctgaggatgccgaagcagtgttcaatcataacggaacaaacggctccgctaatgtctctgctaatgaccatggttgcagaaatttcttgcatctgttgcgattaagcggcaatggtttggaaattaatcggggacgaaccgaatggagaaagaaacccacacggtagtctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaaatgcgagactacgagcatgttgtggaaagttttgactacttacaatccgcaactcaagaccttcacggagagttgaccgccctgaatgcttgtgttgagtgttgtgatagacacgcgcatggtgaagctgttgccctttactgtgaggcccaagacggtcacgcggaacgatatcggttccgagatttgcagcgtcaagccgcacgatttggtaactttcttcgagagcagggagtgaaacctggtgaccgagtggctggtttgatgccccgtaccgtcgagttgcttatcgctatcctgggaacatggcggattggagctgtgtatcagcccttgttcaccgcgtttggtccgaaagctattgagcagcggctgaattgtagcaatgctcgatggattgtcactgatccccataatcgaccaaaattggatgatgttaccgattgtccgagtatagtcgtcactggcggtgcaccccaaaatccggcagaccatcacttttggtccgccttgaatcgacaagccgatgactgtgctcctgtcttattagacgcttccgctcccttcttgctcatgtgcacttcgggtactaccggccctgcgaagcctttggaagtccccctctcggccatcttagcttttaaaggttacatgcgagatgccatcgatcttagagcagatgatcgattttggaatttggccgatccaggatgggcgtacggactgtactatgccgtcactggacccttggcctgcggctatgccaccttgttttatgatggaccgtttaccgttgagtccactcgacatataatcgctaaatatgccataaataatttggctggatcaccgacagcttatcgattcttgattgctgccggcgctgaatttgctgatgcagtgcgcggtcgattgcgtgctgtgtctagtgcgggcgagcccttgaatccccaggtggttcgatggttcgctgagcaactgggagtcgtcatccatgatcattatggtcagacagagataggtatggtcctttgtaaccatcatggacttcgacatcccgtgcgtgagggttcggcaggatacgcagttcctggttatcgtattgtggtcctcgacaaagctcatcgagagctgccggcaggtcaacctggagttttggcagtcgaccgagaacgatcccctctgtgttggtttgatggttatttgggaatgcctacacaagcgttcgccggacgatactatttgtccggagacatcgtcgaactgaacgatgatggttctatttcttttgttggtcgaaacgacgatttgatcactacctctggatatcgagtgggtccatttgacgtcgagtccgccttgattgagcatcccgctgtcgttgaagctgcagttataggaaagccagaccctcagcggacagaactgattaaagccttcgttgtcttgaatactccatatctcccttcgccagaattagccgaagagttgcgtttgcacgtgcgacaacggttggctgcccacgcatacccccgagagatggaattcgttgatcacttaccgaaaacaccttcgggaaaactgcagcgatttatcttgagaaatcaagaaatcgcgaaacagcaagccttgggatagggattgtctgtggtatctaagctatttatcactctttacaacttctacctcaactatctactttaataaatgaatatcgtttattctctatgattactgtatatgcgttcctctaagacaaatcggtcacgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccccaccacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtaggtatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 93 - DNA, 9103 bp, Artificial Sequence
  Nucleotide sequence of the NotI DNA fragment of pAT208 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgctgaaattatcttcttcacgttcgccgttggcaagaattccgacccgacctcgacctaattccatcccgcccagaatcattgttgttagcagttcttctagcaaggttaatcctttgaaaactgaagctgttgtcagctcgggattggcggatagattgcgactgggttcgttgaccgaagatggtttatcctacaaagagaaatttattgtgcgatgctatgaggtgggcatcaataaaacagccaccgtggagactattgcaaacttgctccaagaagtcggttgtaaccacgcccaatccgtgggctattccactggcggcttttctactaccccgaccatgcgaaaattgcggctgatttgggtcactgccagaatgcatatcgagatatataaatatcccgcctggtctgatgtcgttgagattgaatcgtggggacaaggtgaaggcaagattggtacacgccgagactggatcttacgtgattatgctaccggtcaagttatcggacgagcgacatcgaagtgggttatgatgaatcaagatacccgacgacttcaaaaggtcgatgtggatgtgcgggatgagtacctcgtgcattgtccgcgtgagttgcgtttggcgttccccgaggagaataattctagcttgaagaagatctccaagttggaagatccttcccaatattcgaaactgggcttggtgccacgacgtgccgacttggatatgaaccaacacgtcaataatgttacttatattggatgggtcctggaatctatgcctcaagagatcatagacactcatgaattgcagacaattaccttggattatcgacgcgagtgccaacatgatgatgttgtggatagcttgacctctcccgaaccctctgaggatgccgaagcagtgttcaatcataacggaacaaacggctccgctaatgtctctgctaatgaccatggttgcagaaatttcttgcatctgttgcgattaagcggcaatggtttggaaattaatcggggacgaaccgaatggagaaagaaacccacacggtagtctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaaatgcgtgactacgaacatgttgtggagtcttttgattacttgcaatctgcaacacaagatctccacggtgagttgaccgcactgaatgcttgtgtggagtgttgtgatcgacacgcgcatggagaagctgttgccctctactgtgaggcccaagacggacacgcggaacgttatagattccgagatctccagcgacaagctgctcgttttggtaactttctcagagagcagggtgttaaacccggcgaccgagttgcaggattaatgccccgaaccgtggagttgcttatcgctatcctgggaacttggagaattggagccgtttatcagcctttattcacagcttttggaccaaaagctattgagcagcgactgaattgttctaatgctcgatggattgtcacagatcctcataatcgacctaaattggatgatgttacagattgtccttcgattgtcgtcacaggcggagctcctcaaaatccagccgaccatcatttctggtcagctctcaatcgacaagccgatgactgtgcccctgttctcttggacgcttcggctccctttctgctgatgtgcacttccggtacaaccggtcccgctaagcccttagaagtccctttgtctgccatattagcatttaaaggatacatgcgcgacgctatagatctgcgagccgatgatcgattttggaatttagccgatcccggctgggcgtacggcttatactatgctgtcactggacccttggcatgcggctatgctacacttttctatgatggtccgtttacagtcgagagcacacgccatattatcgcgaaatatgctataaataatttggctggaagcccaactgcttatcgatttctcattgctgccggtgctgaatttgctgatgctgtccggggccgtttgcgagctgtgtcctctgctggcgagcctttgaatcctcaggtggtcagatggttcgctgagcaattgggagttgtcatacatgatcattatggtcagacagagattggtatggttttgtgtaaccatcatggactgcgacatcccgtgcgtgagggttctgccggatacgctgtccctggttatagaattgtggtcctcgacaaagcccatcgtgagctccccgctggtcaacccggagtgttggcagtggaccgtgaacggtcccccttatgttggtttgatggatatttgggtatgccaactcaagctttcgctggacgttactatttgtctggagacatcgtcgaattgaacgatgatggttctatttcgtttgttggacggaacgacgatctcatcacaacctcaggatatagagtgggcccatttgacgtcgagtccgccttaatagagcatccggctgtcgtggaagccgctgtcattggaaagccagacccccagagaactgaactgatcaaagccttcgtggtcttgaataccccttatttaccttctccggaattagccgaagagttgcgattgcacgtgagacaacggttagcggctcacgcatacccccgcgagatggaattcgtggatcacttgccaaagacacccagcggtaaactgcagagatttatcttgcggaatcaagaaatcgcaaaacagcaagctttaggctagggattgtctgtggtatctaagctatttatcactctttacaacttctacctcaactatctactttaataaatgaatatcgtttattctctatgattactgtatatgcgttcctctaagacaaatcggtcacgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccccaccacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtaggtatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 94 - DNA, 9112 bp, Artificial Sequence
  Nucleotide sequence of the NotI DNA fragment of pAT209 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaaatgcgagactacgagcatgttgtggaaagttttgactacttacaatccgcaactcaagaccttcacggagagttgaccgccctgaatgcttgtgttgagtgttgtgatagacacgcgcatggtgaagctgttgccctttactgtgaggcccaagacggtcacgcggaacgatatcggttccgagatttgcagcgtcaagccgcacgatttggtaactttcttcgagagcagggagtgaaacctggtgaccgagtggctggtttgatgccccgtaccgtcgagttgcttatcgctatcctgggaacatggcggattggagctgtgtatcagcccttgttcaccgcgtttggtccgaaagctattgagcagcggctgaattgtagcaatgctcgatggattgtcactgatccccataatcgaccaaaattggatgatgttaccgattgtccgagtatagtcgtcactggcggtgcaccccaaaatccggcagaccatcacttttggtccgccttgaatcgacaagccgatgactgtgctcctgtcttattagacgcttccgctcccttcttgctcatgtgcacttcgggtactaccggccctgcgaagcctttggaagtccccctctcggccatcttagcttttaaaggttacatgcgagatgccatcgatcttagagcagatgatcgattttggaatttggccgatccaggatgggcgtacggactgtactatgccgtcactggacccttggcctgcggctatgccaccttgttttatgatggaccgtttaccgttgagtccactcgacatataatcgctaaatatgccataaataatttggctggatcaccgacagcttatcgattcttgattgctgccggcgctgaatttgctgatgcagtgcgcggtcgattgcgtgctgtgtctagtgcgggcgagcccttgaatccccaggtggttcgatggttcgctgagcaactgggagtcgtcatccatgatcattatggtcagacagagataggtatggtcctttgtaaccatcatggacttcgacatcccgtgcgtgagggttcggcaggatacgcagttcctggttatcgtattgtggtcctcgacaaagctcatcgagagctgccggcaggtcaacctggagttttggcagtcgaccgagaacgatcccctctgtgttggtttgatggttatttgggaatgcctacacaagcgttcgccggacgatactatttgtccggagacatcgtcgaactgaacgatgatggttctatttcttttgttggtcgaaacgacgatttgatcactacctctggatatcgagtgggtccatttgacgtcgagtccgccttgattgagcatcccgctgtcgttgaagctgcagttataggaaagccagaccctcagcggacagaactgattaaagccttcgttgtcttgaatactccatatctcccttcgccagaattagccgaagagttgcgtttgcacgtgcgacaacggttggctgcccacgcatacccccgagagatggaattcgttgatcacttaccgaaaacaccttcgggaaaactgcagcgatttatcttgagaaatcaagaaatcgcgaaacagcaagccttgggatagggattgtctgtggtatctaagctatttatcactctttacaacttctacctcaactatctactttaataaatgaatatcgtttattctctatgattactgtatatgcgttcctctaagacaaatcggtcacgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccccaccacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtaggtatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 95 - DNA, 9112 bp, Artificial Sequence
  Nucleotide sequence of the NotI DNA fragment of pAT210 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaaatgcgtgactacgaacatgttgtggagtcttttgattacttgcaatctgcaacacaagatctccacggtgagttgaccgcactgaatgcttgtgtggagtgttgtgatcgacacgcgcatggagaagctgttgccctctactgtgaggcccaagacggacacgcggaacgttatagattccgagatctccagcgacaagctgctcgttttggtaactttctcagagagcagggtgttaaacccggcgaccgagttgcaggattaatgccccgaaccgtggagttgcttatcgctatcctgggaacttggagaattggagccgtttatcagcctttattcacagcttttggaccaaaagctattgagcagcgactgaattgttctaatgctcgatggattgtcacagatcctcataatcgacctaaattggatgatgttacagattgtccttcgattgtcgtcacaggcggagctcctcaaaatccagccgaccatcatttctggtcagctctcaatcgacaagccgatgactgtgcccctgttctcttggacgcttcggctccctttctgctgatgtgcacttccggtacaaccggtcccgctaagcccttagaagtccctttgtctgccatattagcatttaaaggatacatgcgcgacgctatagatctgcgagccgatgatcgattttggaatttagccgatcccggctgggcgtacggcttatactatgctgtcactggacccttggcatgcggctatgctacacttttctatgatggtccgtttacagtcgagagcacacgccatattatcgcgaaatatgctataaataatttggctggaagcccaactgcttatcgatttctcattgctgccggtgctgaatttgctgatgctgtccggggccgtttgcgagctgtgtcctctgctggcgagcctttgaatcctcaggtggtcagatggttcgctgagcaattgggagttgtcatacatgatcattatggtcagacagagattggtatggttttgtgtaaccatcatggactgcgacatcccgtgcgtgagggttctgccggatacgctgtccctggttatagaattgtggtcctcgacaaagcccatcgtgagctccccgctggtcaacccggagtgttggcagtggaccgtgaacggtcccccttatgttggtttgatggatatttgggtatgccaactcaagctttcgctggacgttactatttgtctggagacatcgtcgaattgaacgatgatggttctatttcgtttgttggacggaacgacgatctcatcacaacctcaggatatagagtgggcccatttgacgtcgagtccgccttaatagagcatccggctgtcgtggaagccgctgtcattggaaagccagacccccagagaactgaactgatcaaagccttcgtggtcttgaataccccttatttaccttctccggaattagccgaagagttgcgattgcacgtgagacaacggttagcggctcacgcatacccccgcgagatggaattcgtggatcacttgccaaagacacccagcggtaaactgcagagatttatcttgcggaatcaagaaatcgcaaaacagcaagctttaggctagggattgtctgtggtatctaagctatttatcactctttacaacttctacctcaactatctactttaataaatgaatatcgtttattctctatgattactgtatatgcgttcctctaagacaaatcggtcacgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccccaccacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtaggtatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 96 - DNA, 8417 bp, Artificial Sequence
  Nucleotide sequence of the NotI DNA fragment of pAT211 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgctgaaattatcttcttcacgttcgccgttggcaagaattccgacccgacctcgacctaattccatcccgcccagaatcattgttgttagcagttcttctagcaaggttaatcctttgaaaactgaagctgttgtcagctcgggattggcggatagattgcgactgggttcgttgaccgaagatggtttatcctacaaagagaaatttattgtgcgatgctatgaggtgggcatcaataaaacagccaccgtggagactattgcaaacttgctccaagaagtcggttgtaaccacgcccaatccgtgggctattccactggcggcttttctactaccccgaccatgcgaaaattgcggctgatttgggtcactgccagaatgcatatcgagatatataaatatcccgcctggtctgatgtcgttgagattgaatcgtggggacaaggtgaaggcaagattggtacacgccgagactggatcttacgtgattatgctaccggtcaagttatcggacgagcgacatcgaagtgggttatgatgaatcaagatacccgacgacttcaaaaggtcgatgtggatgtgcgggatgagtacctcgtgcattgtccgcgtgagttgcgtttggcgttccccgaggagaataattctagcttgaagaagatctccaagttggaagatccttcccaatattcgaaactgggcttggtgccacgacgtgccgacttggatatgaaccaacacgtcaataatgttacttatattggatgggtcctggaatctatgcctcaagagatcatagacactcatgaattgcagacaattaccttggattatcgacgcgagtgccaacatgatgatgttgtggatagcttgacctctcccgaaccctctgaggatgccgaagcagtgttcaatcataacggaacaaacggctccgctaatgtctctgctaatgaccatggttgcagaaatttcttgcatctgttgcgattaagcggcaatggtttggaaattaatcggggacgaaccgaatggagaaagaaacccacacggtagtctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaacacaatgtctatcggatcttctaaccccgtgctgctggccgctattcccttcgtgtacctgttcgtgctgccccgagtgctggccttcctgcctcagaaggcccagttcctggccaagtgcatcgtggtgctgatcgctaccctgatcatgtctgtggccggctgcttcatctctatcgtgtgcgccctgctggacaagcgatacgtgatcaactacgtggtgtctcgactgttctctttcctggccgctcgaccctgcggcgtgacctacaagatcgtcggcgaggaacacctggacaagtaccccgccattgtggtgtgcaaccaccagtcctctatggacatgatggtgctgggccgagtgttccccaagcactgcgtggtgatggccaagaaggaactgctgtacttccccttcctgggcatgttcatgaagctgtctaacgccatcttcatcgaccgaaagaaccacaagaaggccatcgagtctaccactcaggccgtggccgacatgaagaagcacaactctggcatctggatcttccccgagggcacccgatctcgactggacaaggccgacctgctgcctttcaagaagggcgccttccacctggccatccaggctcagctgcccattctgcccatcatctctcagggctactctcacatctacgactcttctaagcgatacttccccggtggcgagctggaaatccgagtgctcgagcccattcctaccaccggcctgaccaccgacgacgtgaacgacctgatggacaagacccgaaacctgatgctgaagcacctgaaggaaatggactctcagtactcttcgtctaccgccgagaacggctctacccacatcgacgccgacattgccaagtccaccgccacctctatcggcaacactgacgacgccattaccaagcgacgaacccctaaggaatagtaaggattgtctgtggtatctaagctatttatcactctttacaacttctacctcaactatctactttaataaatgaatatcgtttattctctatgattactgtatatgcgttcctctaagacaaatcggtcacgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccccaccacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtaggtatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 97 - DNA, 8426 bp, Artificial Sequence
  Nucleotide sequence of the NotI DNA fragment of pAT212 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaacacaatgtctatcggatcttctaaccccgtgctgctggccgctattcccttcgtgtacctgttcgtgctgccccgagtgctggccttcctgcctcagaaggcccagttcctggccaagtgcatcgtggtgctgatcgctaccctgatcatgtctgtggccggctgcttcatctctatcgtgtgcgccctgctggacaagcgatacgtgatcaactacgtggtgtctcgactgttctctttcctggccgctcgaccctgcggcgtgacctacaagatcgtcggcgaggaacacctggacaagtaccccgccattgtggtgtgcaaccaccagtcctctatggacatgatggtgctgggccgagtgttccccaagcactgcgtggtgatggccaagaaggaactgctgtacttccccttcctgggcatgttcatgaagctgtctaacgccatcttcatcgaccgaaagaaccacaagaaggccatcgagtctaccactcaggccgtggccgacatgaagaagcacaactctggcatctggatcttccccgagggcacccgatctcgactggacaaggccgacctgctgcctttcaagaagggcgccttccacctggccatccaggctcagctgcccattctgcccatcatctctcagggctactctcacatctacgactcttctaagcgatacttccccggtggcgagctggaaatccgagtgctcgagcccattcctaccaccggcctgaccaccgacgacgtgaacgacctgatggacaagacccgaaacctgatgctgaagcacctgaaggaaatggactctcagtactcttcgtctaccgccgagaacggctctacccacatcgacgccgacattgccaagtccaccgccacctctatcggcaacactgacgacgccattaccaagcgacgaacccctaaggaatagtaaggattgtctgtggtatctaagctatttatcactctttacaacttctacctcaactatctactttaataaatgaatatcgtttattctctatgattactgtatatgcgttcctctaagacaaatcggtcacgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccccaccacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtaggtatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 98 - DNA, 20 bp, Artificial Sequence
  Nucleotide sequence of artificial oligonucleotide primer at 001
tgctggcgtaggtgaagtcg

SEQ ID NO: 99 - DNA, 20 bp, Artificial Sequence
  Nucleotide sequence of artificial oligonucleotide primer at 002
gatcagtgcatgccggatcg

SEQ ID NO: 100 - DNA, 35 bp, Artificial Sequence
  Nucleotide sequence of artificial oligonucleotide primer at 085
gggggtctctacaaatgcgagactacgagcatgtt

SEQ ID NO: 101 - DNA, 35 bp, Artificial Sequence
  Nucleotide sequence of artificial oligonucleotide primer at 086
cccggtctctatccctatcccaaggcttgctgttt

SEQ ID NO: 102 - DNA, 37 bp, Artificial Sequence
  Nucleotide sequence of artificial oligonucleotide primer at 274
gggggtctctaatgcacaatgtctatctctaactctc

SEQ ID NO: 103 - DNA, 36 bp, Artificial Sequence
  Nucleotide sequence of artificial oligonucleotide primer at 275
cccggtctcttagactacttagaaacgccgttttcc

SEQ ID NO: 104 - DNA, 37 bp, Artificial Sequence
  Nucleotide sequence of artificial oligonucleotide primer at 297
gggggtctctacaacacaatgtctatctctaactctc

SEQ ID NO: 105 - DNA, 36 bp, Artificial Sequence
  Nucleotide sequence of artificial oligonucleotide primer at 298
cccggtctctatccctacttagaaacgccgttttcc

SEQ ID NO: 106 - DNA, 39 bp, Artificial Sequence
  Nucleotide sequence of artificial oligonucleotide primer at 299
gggggtctctccaccacaatgactatcgactcacaatac

SEQ ID NO: 107 - DNA, 37 bp, Artificial Sequence
  Nucleotide sequence of artificial oligonucleotide primer at 300
cccggtctctatacttactcaatcattcggaactctg

SEQ ID NO: 108 - DNA, 4655 bp, Artificial Sequence
  Nucleotide sequence of the NotI DNA fragment of pAT091 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgcgagactacgagcatgttgtggaaagttttgactacttacaatccgcaactcaagaccttcacggagagttgaccgccctgaatgcttgtgttgagtgttgtgatagacacgcgcatggtgaagctgttgccctttactgtgaggcccaagacggtcacgcggaacgatatcggttccgagatttgcagcgtcaagccgcacgatttggtaactttcttcgagagcagggagtgaaacctggtgaccgagtggctggtttgatgccccgtaccgtcgagttgcttatcgctatcctgggaacatggcggattggagctgtgtatcagcccttgttcaccgcgtttggtccgaaagctattgagcagcggctgaattgtagcaatgctcgatggattgtcactgatccccataatcgaccaaaattggatgatgttaccgattgtccgagtatagtcgtcactggcggtgcaccccaaaatccggcagaccatcacttttggtccgccttgaatcgacaagccgatgactgtgctcctgtcttattagacgcttccgctcccttcttgctcatgtgcacttcgggtactaccggccctgcgaagcctttggaagtccccctctcggccatcttagcttttaaaggttacatgcgagatgccatcgatcttagagcagatgatcgattttggaatttggccgatccaggatgggcgtacggactgtactatgccgtcactggacccttggcctgcggctatgccaccttgttttatgatggaccgtttaccgttgagtccactcgacatataatcgctaaatatgccataaataatttggctggatcaccgacagcttatcgattcttgattgctgccggcgctgaatttgctgatgcagtgcgcggtcgattgcgtgctgtgtctagtgcgggcgagcccttgaatccccaggtggttcgatggttcgctgagcaactgggagtcgtcatccatgatcattatggtcagacagagataggtatggtcctttgtaaccatcatggacttcgacatcccgtgcgtgagggttcggcaggatacgcagttcctggttatcgtattgtggtcctcgacaaagctcatcgagagctgccggcaggtcaacctggagttttggcagtcgaccgagaacgatcccctctgtgttggtttgatggttatttgggaatgcctacacaagcgttcgccggacgatactatttgtccggagacatcgtcgaactgaacgatgatggttctatttcttttgttggtcgaaacgacgatttgatcactacctctggatatcgagtgggtccatttgacgtcgagtccgccttgattgagcatcccgctgtcgttgaagctgcagttataggaaagccagaccctcagcggacagaactgattaaagccttcgttgtcttgaatactccatatctcccttcgccagaattagccgaagagttgcgtttgcacgtgcgacaacggttggctgcccacgcatacccccgagagatggaattcgttgatcacttaccgaaaacaccttcgggaaaactgcagcgatttatcttgagaaatcaagaaatcgcgaaacagcaagccttgggatagtctagtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 109 - DNA, 4085 bp, Artificial Sequence
  Nucleotide sequence of the NotI DNA fragment of pAT108 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctagtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 110 - DNA, 4629 bp, Artificial Sequence
  Nucleotide sequence of the NotI DNA fragment of pAT135 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgcacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtagtctagtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 111 - DNA, 6721 bp, Artificial Sequence
  Nucleotide sequence of the NotI DNA fragment of pAT213 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaaatgcgagactacgagcatgttgtggaaagttttgactacttacaatccgcaactcaagaccttcacggagagttgaccgccctgaatgcttgtgttgagtgttgtgatagacacgcgcatggtgaagctgttgccctttactgtgaggcccaagacggtcacgcggaacgatatcggttccgagatttgcagcgtcaagccgcacgatttggtaactttcttcgagagcagggagtgaaacctggtgaccgagtggctggtttgatgccccgtaccgtcgagttgcttatcgctatcctgggaacatggcggattggagctgtgtatcagcccttgttcaccgcgtttggtccgaaagctattgagcagcggctgaattgtagcaatgctcgatggattgtcactgatccccataatcgaccaaaattggatgatgttaccgattgtccgagtatagtcgtcactggcggtgcaccccaaaatccggcagaccatcacttttggtccgccttgaatcgacaagccgatgactgtgctcctgtcttattagacgcttccgctcccttcttgctcatgtgcacttcgggtactaccggccctgcgaagcctttggaagtccccctctcggccatcttagcttttaaaggttacatgcgagatgccatcgatcttagagcagatgatcgattttggaatttggccgatccaggatgggcgtacggactgtactatgccgtcactggacccttggcctgcggctatgccaccttgttttatgatggaccgtttaccgttgagtccactcgacatataatcgctaaatatgccataaataatttggctggatcaccgacagcttatcgattcttgattgctgccggcgctgaatttgctgatgcagtgcgcggtcgattgcgtgctgtgtctagtgcgggcgagcccttgaatccccaggtggttcgatggttcgctgagcaactgggagtcgtcatccatgatcattatggtcagacagagataggtatggtcctttgtaaccatcatggacttcgacatcccgtgcgtgagggttcggcaggatacgcagttcctggttatcgtattgtggtcctcgacaaagctcatcgagagctgccggcaggtcaacctggagttttggcagtcgaccgagaacgatcccctctgtgttggtttgatggttatttgggaatgcctacacaagcgttcgccggacgatactatttgtccggagacatcgtcgaactgaacgatgatggttctatttcttttgttggtcgaaacgacgatttgatcactacctctggatatcgagtgggtccatttgacgtcgagtccgccttgattgagcatcccgctgtcgttgaagctgcagttataggaaagccagaccctcagcggacagaactgattaaagccttcgttgtcttgaatactccatatctcccttcgccagaattagccgaagagttgcgtttgcacgtgcgacaacggttggctgcccacgcatacccccgagagatggaattcgttgatcacttaccgaaaacaccttcgggaaaactgcagcgatttatcttgagaaatcaagaaatcgcgaaacagcaagccttgggatagggatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 112 - DNA, 6695 bp, Artificial Sequence
  Nucleotide sequence of the NotI DNA fragment of pAT214 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaacacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtagggatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 113 - DNA, 7265 bp, Artificial Sequence
  Nucleotide sequence of the NotI DNA fragment of pAT215 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgcgagactacgagcatgttgtggaaagttttgactacttacaatccgcaactcaagaccttcacggagagttgaccgccctgaatgcttgtgttgagtgttgtgatagacacgcgcatggtgaagctgttgccctttactgtgaggcccaagacggtcacgcggaacgatatcggttccgagatttgcagcgtcaagccgcacgatttggtaactttcttcgagagcagggagtgaaacctggtgaccgagtggctggtttgatgccccgtaccgtcgagttgcttatcgctatcctgggaacatggcggattggagctgtgtatcagcccttgttcaccgcgtttggtccgaaagctattgagcagcggctgaattgtagcaatgctcgatggattgtcactgatccccataatcgaccaaaattggatgatgttaccgattgtccgagtatagtcgtcactggcggtgcaccccaaaatccggcagaccatcacttttggtccgccttgaatcgacaagccgatgactgtgctcctgtcttattagacgcttccgctcccttcttgctcatgtgcacttcgggtactaccggccctgcgaagcctttggaagtccccctctcggccatcttagcttttaaaggttacatgcgagatgccatcgatcttagagcagatgatcgattttggaatttggccgatccaggatgggcgtacggactgtactatgccgtcactggacccttggcctgcggctatgccaccttgttttatgatggaccgtttaccgttgagtccactcgacatataatcgctaaatatgccataaataatttggctggatcaccgacagcttatcgattcttgattgctgccggcgctgaatttgctgatgcagtgcgcggtcgattgcgtgctgtgtctagtgcgggcgagcccttgaatccccaggtggttcgatggttcgctgagcaactgggagtcgtcatccatgatcattatggtcagacagagataggtatggtcctttgtaaccatcatggacttcgacatcccgtgcgtgagggttcggcaggatacgcagttcctggttatcgtattgtggtcctcgacaaagctcatcgagagctgccggcaggtcaacctggagttttggcagtcgaccgagaacgatcccctctgtgttggtttgatggttatttgggaatgcctacacaagcgttcgccggacgatactatttgtccggagacatcgtcgaactgaacgatgatggttctatttcttttgttggtcgaaacgacgatttgatcactacctctggatatcgagtgggtccatttgacgtcgagtccgccttgattgagcatcccgctgtcgttgaagctgcagttataggaaagccagaccctcagcggacagaactgattaaagccttcgttgtcttgaatactccatatctcccttcgccagaattagccgaagagttgcgtttgcacgtgcgacaacggttggctgcccacgcatacccccgagagatggaattcgttgatcacttaccgaaaacaccttcgggaaaactgcagcgatttatcttgagaaatcaagaaatcgcgaaacagcaagccttgggatagtctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaacacaatgtctatctctaactctcccgagaacctggaccagaagcctaacatccgaaacgagtctgaggaccgatctttctctgtgcgacgacgaactaactctaacgtggtgaacgctgtgcccgactcttgctctaaggtgggcaccatcgagactgtgaccaagttctcttctgtggactctgccgccgagatgtcctctggcgaggaccgaaaccgaatcggcaacggtgacgaccgaaaggaagaggccaactctctgtctaacggccaggtgtacaacgagcgagtcggcatcggcgagggacgatctcccgaggccgaggtgcccttccgattctcttaccgagcctctgctcccgctcaccgacgagtgaaggaatctcccctgtcctctgacgccatcttccgacagtctcacgccggcctgttcaacctgtgcatcctggtgctggtggccgtgaacggccgactgatcattgagaacctgatgaagtacggcctgctgatccgagccggattctggttctcttcgaagtctctgcgagactggcccctgctgatgtgctgcctgtctctgcctatcttccctctgggcgccttcaccgtcgagaagtctgccaagcacaagttcatccccgagcctgtggtgatgtctctgcacgtgctgatctgtaccgcctctctgctgtaccccatcttcgtgatcctccgatgcggatctgccgtgctgttcggaatcgccctgatgctgctggcctctatcgtgtggctgaagctggtgtcttacgcccacaccaactgcgacatgcgagccctggccaagctgaccgacaaggtggcctctaagtcctccgacatggactactcttacgacgtgtctttcgagtctctggcctacttcatggtggctcccactctgtgctaccaggcttcttaccctcgatctacctgcatccgaaagggctgggtgatccgacagctggtcaagctggtgattttcaccggcgtgatgggcttcatcgtcgagcagtacatcaaccccatcgtgcagcactctcagcaccctctgaagggcaacctgctgtacgctatcgagcgagtgctgaagctctctgtccctactctgtacgtctggctgtgcatgttctactgtttcttccacctgtggctcaacatcctggccgagctgctgcgattcggcgaccgagagttctacaaggactggtggaacgccaagaccgtggaagagtactggcgaatgtggaacatgcccgtgcacaagtggatggtgcgacacatctacttcccttgcctgcgaaacggactgccccgagaggtgaccctggtgatcgtgttcttcctgtctgccattttccacgagctgtgcatcggcgtgccctgccacatcttcaagttctgggccttcatcggcatcatgttccaggtgcctctggtgctgatcaccaactacctgcagaacaagttccgatcttctatggtgggcaacatggtgttctggtttttcttctcgatcctgggacagcccatgtctgtgctgctgtactaccacgacgtgatgaacagaaaggaaaacggcgtttctaagtagggatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 114 - DNA, 9267 bp, Artificial Sequence
  Nucleotide sequence of the NotI DNA fragment of pAT216 used to transform Y. lipolytica cells.
gcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtataacttcgtatagcatacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtataacttcgtatagcaggagttatccgaagcgaacggcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccaatgatgttcaaaatttcgtcgtcactctcacctgttgaccaaattcccccaattagccctttgcccagaccacgaccccgtccaattacaccaagagttctggcagtgtccagctccagcggcaaaatcgttaataatcccttgaaagccgaaaccaccgaagcggtcagtggcgaactggcccgcagatttagattgggtcgactggctgaagatggatttagctataaagagaaattcatcgtcagatgttatgaagttggtattaataaaactgcgacagtcgaaaccttggctaatttgctgcaagaagtgggtggaaatcatgcccaatcggttggctttagcactgatggcttcgctaccactcattccatgcggaaaatgcatttgatttgggtgaccgctcggatgcatatagaaatctataaatatcctgcctggtcggacgtcattgaagtcgaaacttggatcggagccgaaggtcgtattggtaccagacgaaattggattattaaagactgcgcgacagatgaagttattggtcgggctacaagcaaatgggtcatgatgaatcaagacacacggcgattggaaaaggtttcggatgatgttcgtgaagaacatttggtcttttcccccagagaaccgcgcctgccgttccccgacgaaaataactcttcattgaagaaaatctcgaaattagatgatccggctcaatattccaaattgagtttggaaccacgacggggagacttagatatgaatcaacatgttaacaatgttacttatataggctgggttctcgagagtatgccacaagagatcattgacacacatgaattgcagacaataactttggactaccggcgagaatgtcaacacgatgatttagtggactcgctgacctctcctgagccttctgaattttctgaaactactaatggtagtgccaatgtcagccccaatgataaccgctgtttaaacttcttgcatttgctgagacttagctccgacggctctgaaattaaccggggtcgtactgtgtggagaaagaaacccgcaaaataatctagtgtctgtggtatctaagctatttatcactctttacaacttctacctcaactatctactttaataaatgaatatcgtttattctctatgattactgtatatgcgttcctctaagacaaatcggcttcgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccacaaatgcgagactacgagcatgttgtggaaagttttgactacttacaatccgcaactcaagaccttcacggagagttgaccgccctgaatgcttgtgttgagtgttgtgatagacacgcgcatggtgaagctgttgccctttactgtgaggcccaagacggtcacgcggaacgatatcggttccgagatttgcagcgtcaagccgcacgatttggtaactttcttcgagagcagggagtgaaacctggtgaccgagtggctggtttgatgccccgtaccgtcgagttgcttatcgctatcctgggaacatggcggattggagctgtgtatcagcccttgttcaccgcgtttggtccgaaagctattgagcagcggctgaattgtagcaatgctcgatggattgtcactgatccccataatcgaccaaaattggatgatgttaccgattgtccgagtatagtcgtcactggcggtgcaccccaaaatccggcagaccatcacttttggtccgccttgaatcgacaagccgatgactgtgctcctgtcttattagacgcttccgctcccttcttgctcatgtgcacttcgggtactaccggccctgcgaagcctttggaagtccccctctcggccatcttagcttttaaaggttacatgcgagatgccatcgatcttagagcagatgatcgattttggaatttggccgatccaggatgggcgtacggactgtactatgccgtcactggacccttggcctgcggctatgccaccttgttttatgatggaccgtttaccgttgagtccactcgacatataatcgctaaatatgccataaataatttggctggatcaccgacagcttatcgattcttgattgctgccggcgctgaatttgctgatgcagtgcgcggtcgattgcgtgctgtgtctagtgcgggcgagcccttgaatccccaggtggttcgatggttcgctgagcaactgggagtcgtcatccatgatcattatggtcagacagagataggtatggtcctttgtaaccatcatggacttcgacatcccgtgcgtgagggttcggcaggatacgcagttcctggttatcgtattgtggtcctcgacaaagctcatcgagagctgccggcaggtcaacctggagttttggcagtcgaccgagaacgatcccctctgtgttggtttgatggttatttgggaatgcctacacaagcgttcgccggacgatactatttgtccggagacatcgtcgaactgaacgatgatggttctatttcttttgttggtcgaaacgacgatttgatcactacctctggatatcgagtgggtccatttgacgtcgagtccgccttgattgagcatcccgctgtcgttgaagctgcagttataggaaagccagaccctcagcggacagaactgattaaagccttcgttgtcttgaatactccatatctcccttcgccagaattagccgaagagttgcgtttgcacgtgcgacaacggttggctgcccacgcatacccccgagagatggaattcgttgatcacttaccgaaaacaccttcgggaaaactgcagcgatttatcttgagaaatcaagaaatcgcgaaacagcaagccttgggatagggattgtctgtggtatctaagctatttatcactctttacaacttctacctcaactatctactttaataaatgaatatcgtttattctctatgattactgtatatgcgttcctctaagacaaatcggtcacgatacgcgtatcgatacgcgtgcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgctgaggtgtctcacaagtgccgtgcagtcccgcccccacttgcttctctttgtgtgtagtgtacgtacattatcgagagggttgttcccgcccacctcgatccggcatgcactgatcacgggcaaaagtgcgttcgatagagagggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccccaccacaatgactatcgactcacaatactacaagtcgcgagacaaaaacgacacggcacccaaaatcgcgggaatccgatatgccccgctatcgacaccattactcaaccgatgtgagactttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacgatctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtctcctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtgttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaagtatgtgtctgtggtatctaagctatttatcactctttacaacttctacctcaactatctactttaataaatgaatatcgtttattctctatgattactgtatatgcgttcctctaagacagagtcatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgc

SEQ ID NO: 115 - protein, 501 aa, Theobroma cacao
MAISDSPEILGSTATVTSSSHSDSDLNLLSIRRRTSTTAAGRAPDRDDSGNGEAVDDRDQVESANLMSNVAENANEMPNSSDTRFTYRPRVPAHRRIKESPLSSGAIFKQSHAGLFNLCIVVLVAVNSRLIIENLMKYGWLIRSGFWFSSRSLSDWPLFMCCLTLPIFPLAAFVVEKLVQRNYISEPVVVFLHAIISTTAVLYPVIVNLRCDSAFLSGVALMLFACIVWLKLVSYAHTNNDMRALAKSAEKGDVDPSYDVSFKSLAYFMVAPTLCYQQSYPRTPAVRKSWVVRQFIKLIVFTGLMGFIIEQYINPIVQNSQHPLKGNLLYAIERVLKLSVPNLYVWLCMFYCFFHLWLNILAELLRFGDREFYKDWWNAKTVEEYWRMWNMPVHKWMVRHIYFPCLRNGIPKGVAIVIAFLVSAVFHELCIAVPCHIFKLWAFIGIMFQVPLVLITNYLQDKFRSSMVGNMIFWFIFSILGQPMCVLLYYHDLMNRKGKAD

SEQ ID NO: 116 - protein, 327 aa, Theobroma cacao
MMGEEMEERKATGYREFSGRHEFPSNTMHALLAMGIWLGAIHFNALLLLFSFLFLPFSKFLVVFGLLLLFMILPIDPYSKFGRRLSRYICKHACSYFPITLHVEDIHAFHPDRAYVFGFEPHSVLPIGVVALADLTGFMPLPKIKVLASSAVFYTPFLRHIWTWLGLTPATKKNFSSLLDAGYSCILVPGGVQETFHMEPGSEIAFLRARRGFVRIAMEMGSPLVPVFCFGQSHVYKWWKPGGKFYLQFSRAIKFTPIFFWGIFGSPLPYQHPMHVVVGKPIDVKKNPQPIVEEVIEVHDRFVEALQDLFERHKAQVGFADLPLKIL

SEQ ID NO: 117 - protein, 476 aa, Theobroma cacao
MGSEAQYKNAMALNGEEGEYSEPVSPTGQYFNSSALSIGVLGVLDSEIPIDDSPTMKLLEDVFLPINPRFSSVMVQDENGAKQWKKVEVKLVDHVNVPVFPPGLSPESYDNYLSDYLSKIATEQLPQNRPLWNIHIVKYTTSNAAGSLIFKLHHALGDGYSLMGALLSCLQRAENPSVPLTFPALSSAPNLSENSNSVFRSLYSAFNTISDFGWSLLKSSFLEDDRTMIRSGDPGVQFKPVVISTMTFSLDHIKQIKTKLGVTINDAITGIIFFGTRLYMQGTSSDKLSNGNSTALVLLNTRTIGGYKSVKDMVKPDADSPWGNQFGFLHVSLPELASTAESCNPLEFIWKAQKLIQRKRNSKAVFFTGQLLEGLRKYRGPEATAKYIHSTLKNSSMTISNLIGPVERMALADHPVKSLYFMVVGVPQSLTITMVSYMGKLTVAIGTEKDFVDPQKFKSSVENAFQMMLKAAQAIA

SEQ ID NO: 118 - protein, 485 aa, Theobroma cacao
MACPGTFSDEPLTPAGRLFLQSQTNIIIHCILRGKNPIDIDAVKSTLRSSLMVRHPRFCSLLVRDKNGFEHWRKAQVDIDQHVIVIDKRLDKSDNFLDESRRFSSEEGEGDDDDEAAVNQYVADLSVSSPLSTDKPLWEIHILVPHKCAVFRIHHALGDGISLMSMLMASCRRADDADALPRMVPEKSAEFKDGKGRDWFWLFGILWGFLKMVCFTVMFVMEFVVSSLLVCDRKTVISGGEGVELWPRKLATARFLLEDMRVVKKAIPNTTINDVLFGVVSSGLSRYLDHRTPNALHEGLRMTGVAMVNLRPQTGFQDLSQLMKGAAEARWGNKFGLILLPVYFKKAGNNPLEYVKRAKRMVDRKKHSLEAYFSYRIGDLVMSLLGSKYACLLNYRLLCNTTFTISNIIGPLEEITLAGNPISSIKVNTSSLPQAITMHMLSYAGRAEMQILVAKDIVPDPEFLAKCFEDALLEMKEAVIGTEKE

SEQ ID NO: 119 - protein, 540 aa, Theobroma cacao
MNRKSEAKWVPKLFHQSLISMNSTEIQEIDQVEAKIGTEEASPVSKDEVPLSTGSLIFHEPGINCCIIAIMGYKSKLDPAVIKEGLKQTLIKHPCFSSKLLSAIDQMMNSGKKKWIRTQVNVDNHVILPEVDTVTESPSQFIEDYVTYLTGIPIDLSKPLWELHLVNLKTPEAEAVGIFRIHHSIGDGMSLISLLLACCRKSSDPKALPTLPKQKQADSRYPHPHGFFRLFLAIWSVLRLILNTLVDLLLFVATVVFLKDTKTPLKGSSGVEQNAKKIVHRTVSLDDIKLVKDAMGMTVNDVILGVTEAGLSRYLNRKYGEVDRGKEAEQKSNHLPRNIRLRATALVNIRQTAGIQVSFLSPFSRASEKVEYASQSFLVFKSKVKWGNQIGYICIPFTIALRNDPLDYLRGAKAAGDRKKLSLEAICTHLTNKCVVKLFGSKLSAALVYRVIFNTTMTLSNVVGPVEEISLYGHPIAFIAPSVYGHPQALTCHFQSYMNKMSIVLAVDPNVIPDPRLLCDDLEESLKIFKDAVVLAKDAD

SEQ ID NO: 120 - protein, 510 aa, Theobroma cacao
MESRNGFRWRKQGLKPIETKTGSKGEEENKSSSGQRTAEEEPLSPSARLFHEPNFNVYIIATLGCKTRIYPDVVKANLGHTLLKHPRFSSLQVEDEDNKGEMKWVRTEVDLERHVVVPDLDPNMDLPDKFLEDYIYNLSKTTIDKSQPLWDLHLLNLRTSQSEAVGVFRIHHSLGDGTSLMSLLLACTRQMNDPLALPTIPIRKKQERKNDRRGFWRIMFMLCSVFQVFWNSVVDVFMFMATALLLKDTENPFKGLPGVEFTPRRIVYGTVSLDDIKLVKNAMNTTVNDVALGITQAGLSRYINRIYGGNKNDGAAIEMDNLPKSIRLRSTLLVNIRPSAGIQALADMMEKDAEAKWGNWIGYVLLPFTIAIRDDPLDYVRDAKATIDRKKRSLEAIYTFSIAELVLKLFGVKAASALSHRIMSHTTMCFSNLVGPLEEIGFYGHPIAFLAPSSYGQPHALMINFQSYIDKMTIVLSVDEGTIPNPHQLCDDIVESLGLIKDAVVTRGLV

SEQ ID NO: 121 - protein, 597 aa, Theobroma cacao
MDIIGLRSRSHLGLKQIKVTRDMKEGQSVSGEEEEPLSPMARMFHEPDSNVKEINDALHAIPMILQTLTCSNMNSAYRQGRRVADENNGGELKWVKTEVELEKHVIIPRVDEEMASQGAADKFVEDYIANISKTKISLSIPMWDCHILNIKTSDAESVLVLLVHHSLGDGTSLMSLLISCSRKLSDLLALLTFPAMKKKQMPTTTTTTWLCFWIRLWSFFLLIWNTSVDLLVCVATLFFYKDTPTPLKPSSRRVACTPGRIMRRTFTLDDIKLVKNATNMTVNDVVLAITQAGLSRYLNRKYGKTKRNEAGRKWEDNLPNNIRLRATLFINLRSSPGIYALGEMLKKNSKAEWGNKIGYVLFPFTISLKDNLLDYIRDVKAATDRKKAYLEAKFRLLMAMVFVRFYRTKFAKFPSTTMWFSNVARPQDDITIFGNQVAYIAPSLYGQLVALTVHVVSYAKMISMVLSVDDNIISDPYQLCDDLEESLKLIKTSVVSPLVRVRLSYSRTKSRSTLVILAKFPSTTIWFSNVARPQDEITIFGNQVAYIASSVYGQPKALTIHVVSFAKKISQPVVLLAGHPVPLTPMLPRRAGADASL

SEQ ID NO: 122 - protein, 514 aa, Theobroma cacao
MDIIDLRSGSHLGLKQIKVTREMEEGRSVSGEDNEPLSPMARMFHEPDSNVYIITIVGFKNPIEPNSFKANLVHTLLKHPRFSSVQVADENNGGELKWVQTEVELEKHVIVPKVDEEMASQGAADKFIEDYISNMSKTKISMSIPMWDCHILNLKTSDAESVLVLRVHHSLGDGTSLMSLLISCSRKLFDPLALPTFPAMKKKPIATTTWLCFWIKLWSFFLLIWNTLVDMLMCVATLYFYKDTPTPLKPPSRSVACTPKRIVRRTFSLDDVKLVKNATNTLIWLIRVYLKCFAIGPFVDRQRCRPSNHSSRLISLDISTVNMRHEAGKEWEDNLPNNIRLRATLFINLRSSPGIYALGEMLKKNSKAEWGNKIGYVLFPFTIALKDNPLEYIRDVKEAMDRKKASLEAKFRHLMATVFVRFYRTRLAKFPSTTMWFSNVAGPQDEITIFGNQVTYIAPSLYGQPVALTIHVVSYAKKMSMVLSVDDNIIPDPYQLCDDLEESLKLIKKSVISQ

SEQ ID NO: 123 - protein, 670 aa, Theobroma cacao
MSFLRRRKVTDSSKSQNSDSNAGKEDDKKKQYKSPKRESYSSKKWSCWDSCCWFIGLICSMWWFLLFLYNAMPASIPQYVTEAITGPLPDPPGVKLRKEGLTVNHPVVFVPGIVTGGLELWEGHHCADGLFRKRLWGGSFGELYKRPLCWAEHMSLDNETGLDPPGIRVRPVSGLVAADYFAAGYFVWAVLIANLAQIGYEEKTMYMAAYDWRLSFQNTEVRDQTLSRIKSNIELMVATNGGKKVVVIPHSMGVLYFLHFMKWVEAPVPMGGGGGSDWCAKHIKAVMNIGAPFLGVPKSVSGLFSIEARDIAIARAFAPGFLDKDVFGLQTFQHLMRMTRTWDSTMSMIPKGGDTIWGGLDWSPEGGSFNCSAKKLKNNGTRTGHNANSNLGNMKSVNYGRIISFGKDVAEAHSSKIERVDFRDVMKGGKLANSSNCDIWTEYHEMGNGAIKAVADYKVYTAGSILDLLHFVAPKLMARGGAHFSYGIADNLDDPKYEHYKYWSNPLETKLPSAPDMEIYSMYGVGIPTERAYVYKLTTATDCYIPFQIDTSAEGGSEGSCLKGGVFSVDGDETVPVLSAGFMCAKGWRGKTRFNPSGIRTYIREYNHAPPSNLLEGRGTQSGAHVDIMGNFALIEDIIRVAAGATGQDLGGDRVYSDIFKWSERINLQL

SEQ ID NO: 124 - protein, 690 aa, Theobroma cacao
MASILRFRKLCYVEQAVKCASVGFESFDQSPKIDQKLDKKEEEVISANNFALEIIKKRKQPRKQPKEWRRLDSCCWMIGYLCSTWWLLLFLYHSLPVTLLQVPESPGVRLKREGLAALHPVVLVPGIVTGGLELWEGQPCADGLFRKRLWGGSFTEIFKRPLCWLEHLSLHSETGLDPPGIRVRAVPGLVAADYFAPGYFVWAVLIENLAKIGYEGKNLHMAAYDWRLSFQNTEIRDHALSRLKSKIELMYLTNGYKKVAVVPHSMGVIYFLHFLKWVETPPPIGGGGGPGWCAKHIKAIMNIGPAFLGVPKAVSNIFSAEGKDIAYIRAMAPGLLDSKILGLQTLEHVMRVSRTWDSIVSLVPKGGETIWGNMDWSPEERHACDFSRKRHSQPSPIDNNVNNSDVKRGFRVKDPVQYGRIISFGKTASQLHSSQLPTIDSKEFLRTSASQNLNFSCGEAWTEYDEMSREGIQKVAANKAYTTQTLLDLLRFVAPKMMLRAEAHFSHGIADDLDNPKYNHYKYWSNPLEMKLPDAPDMEIYCLYGVGIPTERSYVYKLSPTSGCKSIPYQIDSSVNGEDGSCLKGGVYFADGDENVPVLSAGFMCAKGWRGRTRFNPSGIATYIREYRHKPPSSLLEGRGIESGAHVDIMGNVALIEDILRIASGATGKEIRGDRIYSDILRMSERINLR

SEQ ID NO: 125 - protein, 685 aa, Theobroma cacao
MSAIRRRKPINESDKTSEASDSKPHKEKEEEQHGDDDGSDKDKKKIPSKIKKKHEEKQPKWSCLDSCCWFIGCICVTWWLLLFLYNAMPASFPQYVTEAITGPLPDPPGVKLKKEGLKAKHPVVFVPGIVTGGLELWEGRKCAEGLFRKRFWGGTFGEVYKRPLCWVEHMSLDNETGLDPCGIRVRPVSGLVAADYFAPGYFVWAVLIANLARIGYEEKTMYMAAYDWRLSFQNTEARDQTLSRIKSNIELIVATNGGKKAVVIPHSMGVLYFLHFMKWVEAPAPMGGGGGPDWCSKHIKAVVNIGGPFLGVPKAIAGLFSAEAKDIAVVRAIAPGFLDNDIFQLQTLQHVMRMSRSWDSTMSMIPRGGDTIWGGLDWSPEEGYSCAKKRERKNDTQIAEEAGVESAVSQTRSAKYGRIISFGKDVAEAPSSDVERIDFRDAVKGHSAANTTCRDVWTEYHDMGFGGINAVAEYKTYTAESIVDLLHFVAPKMMARGTAHFSYGIADNLDDPKYKHYKYWSNPLETKLPNAPEMEIFSLYGVGLPTERAYIYKLSPAAECYIPFQIDTSADDEETCLKDGVYSVDGDETVPVLSAGFMCAKGWRGKTRFNPSGIRTYIREYSHSPPANLLEGRGTLSGAHVDIMGNFALIEDVIRVAAGASGEELGGDQVYSNIFNWSEKIDLQL

SEQ ID NO: 126 - protein, 500 aa, Theobroma cacao
MAPAKSGRSFPSITKCEGSTYESIAADLDGTLLISRSSFPYFMLVAVEAGSLLRGLILLLSLPLVIVSYLFISEAIGIQILIFISFAGLKIRDIELVSRAVLPRFYAANVRKESFEVFDRCKRKVVVTANPTFMVEPFVKDFLGGDKVLGTEIEVNPKTKKATGFVKKPGVLVSELKRLAILKEFGEESPDLGIGDRESDHDFMSICKEGYMVHPSKSATPVPLDRLKSRIIFHDGRFVQRPDPLNALITYLWLPFGFILSIVRVYFNLPLPERIVRYTYEMLGIHLVIRGKRPPPPSPGTPGNLYVCNHRSALDPIVIAIALGRKVSCVTYSVSRLSRFLSPIPAVALTRDRAADAARISELLQKGDLVVCPEGTTCREQFLLRFSALFAELSDRIVPVAVNCRQNMFYGTTVRGVKFWDPYFFFMNPRPTYEVTFLDRLPEEMTVKAGGKSSIEVANHVQKVLGDVLGFECTGLTRKDKYLLLGGNDGKVESMYNAKK

SEQ ID NO: 127 - protein, 376 aa, Theobroma cacao
MSSRGGKLSSSSSELDLDGPNIEDYLPSGSSINEPRGKLRLRDLLDISPTLTEAAGAIVDDSFTRCFKSNPPEPWNWNVYLFPLWCFGVAVRYLILFPARVVVLTIGWIIFLSSFIPVHFLLKGHDKLRKKMERVLVELMCSFFVASWTGVVKYHGPRPSIRPKQVFVANHTSMIDFIILEQMTAFAVIMQKHPGWVGLLQSTILESVGCIWFNRSEAKDREIVAKKLRDHVQGVDNNPLLIFPEGTCINNQYSVMFKKGAFELGCTVCPIAIKYNKIFVDAFWNSRKQSFTMHLLQLMTSWAVVCDVWYLEPQNLRPGETPIEFAERVRDIISVRAGLKKVPWDGYLKYSRPSPKHRERKQQSFAESVLLRLEEK

SEQ ID NO: 128 - protein, 540 aa, Theobroma cacao
MVFPVVFLKLADWVLYQLLANSCYRAARKMRNYGFFLRNQTLRSPPQQQAASLFPSVTKCDVGNSRRFDTLVCDIHGVLLGSDTFFPYFMLVAFEGGSIVRAFLLLLSCSFLWVLDSELKLRIMIFISFCGLRKKDIESVGRAVLPKFYLENLNLQVYEVWSKTSSRVVFTSIPRVMVEGFLHEYMSASGVVGTELHTVGNRFTGLLSSSGLLVKHNALKEHFGDKKPDVGLGSSSLHDQYFISLCKEAYVVNMEDGKSNLSSFMPRDKYPKPLIFHDGRLAFLPTPFATLSMFLWLPFGIVLSILRIFVGICLPYKLAVICATLSGVQLKFQGCFPSSNSQHKKGVLYVCTHRTLLDPVFLSTALCKPLTAVTYSLSKMSELIAPIKTVRLTRDRKQDGETMQKLLSEGDLVVCPEGTTCREPYLLRFSSLFAELADEIVPVAINAHVSMFYGTTASGLKCLDPIFFLMNPRPSYHVQILGKVPQEFTCAGGRSSLEVANYIQRKLADALGFECTTLTRRDKYLMLAGNEGVVHENKRN

SEQ ID NO: 129 - protein, 537 aa, Theobroma cacao
MAKLSMEFSFFQTLFFLFCRVVFRQSKNHKSLHRNVSNIHANEGKYHKYPSFVHRSNLSNQTLVFSVEEALLKSSSLFPYFMLVAFEAGGLLRAFILFVLYPILCLVSEEMGLKIMVLVCFFGIKKKSFRVGSAVLPKFFLEDVGLEPFEMLKKGGKKVAVSKIPQVMIESFLKDYLEIDFVVGRELKEFCGYFLGVMEEKKRSKAALDEIIGSESMGFDVIGISGLKKSLDYHFFSHCKEIYQVRKADKRNWRHVPRQEYSKPLIFHDGRLALRPTLVASLTMFVWFPFGLALSILRAVVGLMLPYKISIPLLAYSGLHLFLSTPESSLHPLSLPNSKKQNPKGRLYVCNHRTLLDPVYLSFALQKDLTAVTYSLSRISELLAPIKTVRLARDRDQDGKMMEKMLNLGDLVVCPEGTTCREPYLLRFSPLFSEMSDDIVPVAMDSNVSLFYGTTASGLKCLDPLFFLMNPRPIYTVQILDGVSGLYTCHDGQRSRFKVANQVQNEIGKALGFECTKLTRRDKYLIMAGNEGIISQT

SEQ ID NO: 130 - protein, 488 aa, Theobroma cacao
MESVVSELEGVLLKDPDPFCYFMLVAFEASGLIRFALLLLFWPLIRLLDMLGLDDAGLKLMTFVATAGLREAEIESVSRAVLPKFYMDDVDMEAWKVFSSYDKRVVVTKTPRIMVARFVKEHLRADEVLGSELVVNRFGFATGFVKGDIASISSRVAKLFVDEEPTLGLGRAPSSFQFLSLCKKQMHPPLMTNQNLHDHQLLRPLPVIFHDGRLVKRPTPSTALLILLWMPLGILLATIRIVVGLILPMRVIPYMSRLFGGKIIVKGKQPPPVSGSSSGVLFVCTHRTLMDPVVLSAVLMRKIPAVTYSISRLSEILSPIPTVRLTRIREVDAEKIKRELAKGDLVVCPEGTTCREPFLLRFSALFAELTDRIVPVAMNYRVGFFHATTARGWKGLDPIFFFMNPRPVYEVTFLNQLPAEATCSSGKSPHDVANYVQRILAATLGFECTNFTRKDKYRVLAGNDGTVSCTSFVDQVKKVVSTFKPFFQ

SEQ ID NO: 131 - protein, 593 aa, Theobroma cacao
MKLSRRQTVLTNDNNNCNLKLAAWPCSFGYKRRKARQTSAIQPKYQVPTASYSSATAQRSKASPRLIHWLAVKDSWKSRALNVLILLFLNMAINAFPTVQNCASIGREKHTMVADMDGTLLISRSSFPYFALVAFEVGGVLRLFFLLLASPLAGLLYYFVSESAGIQVLIFATYVGMKVSDIESVARAVLPKFYSSDLHPESWRVFSSCGKRCVLTANPRIMVEPFLKDFLGADMVLGTEIGIYRGRATGFVCQPGVLVGKNKADALQKAFGEALPDVGLGDRHTDIPFIALCKEGYIVPPKPEAKAVRSEKLPKPVIFHDGRLVQKPTPLMALLIIIWIPIGFLLACLRITAGSLLPMPLVYYAFWALGVRVTVKGTPPPPVKKSTGQSGILFICSHRTLLDPIFLSTALGRPIPVVTYSVSRLSEIISPIKTVSLSRDRATDASMIKKLLQEGDLAICPEGTTCREPFLLRFSALFAELTDQHVPVAMVNRMSMFHGTTARGWKGMDPFYFFMNPSPAYEVTFLNKLPMELTCSSGKSSHEIANYIQRVIAATLSYECTSFTRKDKYRALAGNDGTVVEKPKFSANKVMGC

SEQ ID NO: 132 - protein, 438 aa, Theobroma cacao
MGSRILPSASLFLCSVFLSSFCASCRSSLSIPPLRASFPCASSKSLAPICRLPLPHACGLGILVLFTASSLAMADVVADVVAPVKPKNRASKKFSSRGVDLDVLLEMSSSNSSPLLSPKVFLRDLKRRPLALTKKILSWALEGARSTAFSLGTGGKFHYPARREFFASGRLLFLLLASPLAGTLYYFTSESHDIRVLIFATFVGMKVSDIESVARAVLPKFYSSDLHPGFRFMWEEMCAYGDSEGHSGVKSPGVLVGKNKADALENESHVVPAKAEAGAVSHDKLPKPIVFHDGRLVQNPTPFMALLTILWIPVGFLLACLRIAVGILLPTPLVYYAMWPLGFRIHIKGTPPQPTKKSIGQTVVLFACSHRTLLDPIALSVALCRPIPTATYSISGLSEIISPIKTARLCLDRATDASIIKKLLEKGDLVICPEGTKT

SEQ ID NO: 133 - protein, 507 aa, Theobroma cacao
MAKTKENPFPSIAQCASIGRDKHTVVADMDGTLLRGRSSFPYFALVAFEVGGILRLLFLLLASPVAGILYYFISEAAGIRVLIFATFVGMKVSDIESVARAVLPKFYSSDLHPETWRVFSSCGKSCVLTANPRVMVEAFLKDYLGADMVTGTEIHVFRGRATGLVKSPGILVGKNKVDALENAFKDKPVADIALGDRKTDYPFMKLCKESYVVPAKPKVEAVSHDKLPKPIVFHDGRLVQKPTPFMALLTILWIPVGFLLACLRIAAGALLPMPLVYYAFWALGVRVHIKGTPPPPAKKSIGQTGVLFICSHRTLLDPIFLSTALGRKIPAVTYSLSRLSEVISPIKTIRLSRDRATDASMIKKLLEAGDLVICPEGTTCREPFLLRFSALFAELTNELVPVAMANRMSMFHGTTARGWKGMDPFYFFMNPSPAYEVTFLNKLPHELTCSAGKSSHEVANYIQRMIAASLSYECTSFTRKDKYRALAGNDGTVVEKPKLSPNKVMGC

SEQ ID NO: 134 - protein, 503 aa, Theobroma cacao
MVMGAHRRFEPISKCSNEGRSNQTVAADLDGTLLLSRSAFPYFMLVALEAGSLVRALVLLASVPFVYFTYLFISESVAINTFIFITFAGLKVKDIELVSRSVLPKFYAEDVHPESWRVFSSFGKRYIITASPRIMVEPFVKTFLGADKVIGTELEVTKSGRATGFTIKPGVLVGEHKRAAISKEFGMKLPDLGLGDRETDHDFMSLCKEGYMVPRSKCDPLPRNKLLSPIIFHEGRLVQRPTPVAALLTFLWLPIGFILSLLRVYLNIPLPERIARYNYKILGIKLIVKGNPPPAPKKGQSGVLFVCNHRTVLDPVVTAVALGRKISCVTYSISKFTEIISPIRAVALSREREKDAANIKRLLEEGDLVICPEGTTCREPFLLRFSALFAELTDRIVPVAINTKQTVFHGTTVRGHKLLDPYFVFMNPMPTYEITFLNQLPTELTCKGGKSAIEVANYIQRVLAGTLGFECTNLTRKDKYAMMAGTDGRVPSKKEKEQEKEKA

SEQ ID NO: 135 - protein, 531 aa, Theobroma cacao
MAGKPQNLNAFLFLQKILLTKTWSKIYLQRKASNSHGFQPQFNKYPFLANGLETHSDKALMFHTEGALLKSSSVFSYFFLVAFEAGGLLRAFILFLLHPIICLVGKEWGLKIMVFVCFVGLKEESFRIGRTVLPKFFLEDVGNEGFDMVMKCGGKKIGVTDMPRVMVDCFLKDYLRVEAVVGRELKVVCGHFVGLMEEKKPSGTAFLELDELLGKENMDSSVIGISCFDNSLDQQLFSCCEVLYMVSEADKKNWQALPREKYPRPLIFHDGRLAFRPTPLASLRMFMWIPFGIFLLIVRATVGYLIPYKFSTPFLCFTGLTGTLIRPSSFPDRADDEQKQGGVLYVCNHRTLLDPIYVSIVLDKPFNAVTYSLSRMTELLAPIKTVRLTRNREEDSKMIGKLLKQGDLVICPEGTTCREPYLLRFSPLFAEITDEIVPVAIDFQVSMFYGTTASGFKFLDPFFLLMNPTSHCSASILEKLPKSLTCQGGGKSKFEVANRVQAIIAKALNFECTSLTRRDKYMILAGNQGIV

SEQ ID NO: 136 - protein, 553 aa, Theobroma cacao
MANRQSVLWGLKTVCLVHQHLNSTIYGNLCQQAIKVIKNDDVASGINEPLSILNQRKLPYLRFKVSNGPATQFKFLKHSTLVHRTDELANQTLAFHLEGALLKSCSLFPYFMLMAFEAGGLFRALILLLLYPLVLLIGKELGLRILVFVSFVGIRKEKFRAGTAILPKFFLEDVGCEGFDIVMSYKKRVAVTGMPKFMVEGFLRDYLGIDAVVARELKEFRGYFLGLMEEKMDAGLVISECTHHIGLGCFRKSHDQKIFSHCKEIYLVTEAEKKNWQVLPRKRYPKPLIFHDGRLAFRPTPLAALIMFIWLPFGFLLNITRTIVFISLPFKLSIPLLALSGCIITVSKPEPTTASINGENKPGGMLYVCNHRTLLDPLFLSAVLMKSVCAVTYSISRFSEVISPIKTVRLSRDREKDGKIMKKLLSQGDLVVCPEGTTCREPYLLRFSPLFAEMTDEIVPVAIKLQVSLFYGSTASGLKCLDSTFHLMNPNPMCSVKILNKLPSCQTHNTGGKSKFEVTNHVQNQIAAALGFECTNLTRKDKYAILAGNEGIV

SEQ ID NO: 137 - protein, 461 aa, Theobroma cacao
MSTLPLPFFSGTSTRAPFSFSLKSSPPSAASSSSSSSLVPFQRVELFRSTSTRTTRSFSCVFYSLKAKAMAELVQDKESGVAATGSSGGGRPEVEHSRTFLEARSEQELLSGIRKEVEAGRLPPNIAAGMEELYQNYRNAVFQSGDPSAVEIVLSNMAVAFDRMLLDVEDPFVFEPYHKALREPFDYYMFGQNYIRPLTDFRNSYVGNLSLFYEIEEKLKQGHNVVLISNHQTEADPAIITLLLEKTNPHVAENMIYVAGDRVITDPLCKPFSMGRNLICVYSKKHMYDVPELVEMKRKANTQSLKQMALLLRGGSKIVWIAPSGGRDRPDPLTEEWYPAPFDSSSVDNMRRLIEHSGAPGHIYPLALLCYNIMPPPPQVEKQIGERRNISFHGAGLSVAPKISFQEIAAACEKSEEAKDVYSQALYKSVTKQYNVLKSAVHGKQGLEASTAGVSLSQPWN

SEQ ID NO: 138 - protein, 522 aa, Theobroma cacao
MAKIKKLFPLKALSLLFETLLKSSGKLPYLRFKVSNGPATQFKFLKHSTLVHRTDELANQTLVFHLEGALLKSCSLFPYFMLVAFEAGGLFRALILLLLYPLVLLIGKELGLRILVFVSFVGITKEKFRAGTTILPNFFLEDVGCEGFDIVMSYKKRVAVTGMPKFMIEGFLRDYLGIDAVVARELKEFRGYFLGLVEENMDAGLVISECTHNIGLGCFRKSHDQKIFSHCKEIYLVTEAEKKNWQVLPRKRYPKPLIFHDGRLAFRPTPFAALIMFIWLPFGFLLNITRAIIFISLPFNLSTPLLALSGFIVTVSKPEPTTASINGENKPGGMLYVCNHRTLLDPLFLSGVLMNSVSAVTYSISKFSEVISPIKTVGLSRDREKDGKIMKKLLSQGDLVVCPEGTTCREPYLLRFSPLFAEMTNEIVPVAIKLQVSLFYGSTASGLKCLDSTFHLMNPNPMCSVKILNKLPSCQTHNTGGKSKFEVNNHVQNQIAAALGFECTNLTRKDKYAILAGNEGIV

SEQ ID NO: 139 - protein, 370 aa, Theobroma cacao
MANLRAHVCFLLSFVTFLNLISCHTNLPANHVALFVFGDSLFDPGNNNYINTTFDFQANFRPYGETFFKYPTGRFSDGRLIPDFLAQFAGLPIIPTYLQPGNHKFTDGVNFASGGAGALVESHQGFVVDLETQIRYFKKVEKSLRQELGDEEAKKLLSRAVYLISVGGNDYLTRNSSASDEEYASMVLGNLTVALKEIYKKGGRKFGFPNMMPLGCLPYMKAKAGGPCIDEFTAIAKLHNKELPKTLQKLETQLEGFKYAYYNFYKSVSERLNNPSKYGFKDATTACCGSGLYGGVYSCGGKRGITEFHLCENPSEYLFFDSYHPSEKAYQQFAELMWSGTTDFVWPYNLKTLFEAKTSNLYDHFIIPPP

SEQ ID NO: 140 - protein, 398 aa, Theobroma cacao
MLTYLPLRCLGLKSLVCISFDCGVIGAMAARVKYSWSLALVLLCIVSLSYFGNAADLGASFVFGDSLVDAGNNNYLPTLSKANIPPNGIDFKASGGQPTGRYTNGRTIGDIVGEELGVPNYAVPFLSPNSTGKAILYGVNYASGGGGIMNATGRIFVNRLGLDIQIDFYNTTRKQFDKLLGPSKAKDYIRKSSIFSITIGANDFLNNYLLPVLSIGARISETPDGFIDDMINHLRDQLTRLYQLDARKFVIGNVGPIGCIPYQKTINQLNENECVDLANKLAKQYNSRLQNLLTELNGKLPGATFVVANVYDLVMELITNYEKYGFITASKACCGNGGQFAGIIPCGPTSTMCKDRDKHVFWDPYHPSEAANLIIARQLLYGSTKYISPVNLKELRNL

SEQ ID NO: 141 - protein, 350 aa, Theobroma cacao
MRVSSSFLASLVLVLVFSAANGDPLVPALISFGDSVVDVGNNNNLNTLIKANFPPYGRDFVTHRPTGRFCNGKLATDFTAEYLGFTSYPPAYLSQDARGNDLLTGASFASAASGLYDRTANLCRAIPLTRQVNYYKEYQTKVVNTVGKAKANDIFAGAIHLLSAGSSDYIQNYYINPMLNRVYTPDQFSDILIRSYSTFIQNLYGLGARRIGVTTLPPTGCLPAAITLFGGGSNQCVTRLNQDAVAFNKKLNNTSESLLDKMPGLKLLVFDIYQPLLDMVMKPADNGFFESRRACCGTGTLETSLLCNDRSLGTCTNATSYVFWDGFHPSEAANQVLAGNLLAQGVSLIS

SEQ ID NO: 142 - protein, 388 aa, Theobroma cacao
MAKLISYINFFKVSVILLLSTTSRCFGTPLEKGSALFIFGDSTVDPGNNNYINTIPENRAHYKPYGQNGFFAEPTGRFSDGRVIVDYVAEYAKLPLIPPFLQPSADFTSGANFASGGGGVLPETNQGLVIDLPTQLKNFEEVQKSLVEKLGEAQTKELISEAVYFISIGSNDYMGGYLGNPKMQEFYPPEAYVGMVIGNLTQTLQVLYEKGARKFGFLSLSPLGCLPALRALNFKDNEGKCLEAASALARAHNDALNAVLTNLEHILPGLKYCNSNFYEWLDDRMNNPSKYGFKDGVNACCGTGPYGGVFTCGGTKNVKDYQLCDNADDHVWWDSFHPTERIHEQFAKALWSGHPSSVGPYNLEDLFFNKEKLTIADIVDSPETDPFQ

SEQ ID NO: 143 - protein, 360 aa, Theobroma cacao
MGKLYTIIVVILIFSSTLASSRNVPAIFIFGDSIFDAGNNHFNKNCSVQADFSPYGSTFFHQPTGRFTNGRTVADFISQFIGIDMQKPYLEAQIAVINGSRKDYPSNGINFASAGSGVLQGTNMEQGVISIQEQLRQFQTLVEQNQIDKNLVQKSLFFLESGSNDIFNYFIPFDPPTLDPDAYVQAMMKEVATLIDQIYKLGARRIAVFSLGPVGCVPARALLLGAPINRCYGKMNVMVKKYNKGLESLINNIPIRYPGAIGVYGAIYDIVQRFRAIPARYGFSDVTSACCGNGPLRGKLQCGIEGYQICLNPNEVLFWDYFHPTEHAYKLISEALWGGNKSQIRPVNLKNLADITFTLV

SEQ ID NO: 144 - DNA, 1506 bp, Artificial Sequence
  codon optimised ORF
atggctatctcggacagccccgagattctcggcagcaccgccactgtcacttcttcttcgcactctgattctgacctcaatctccttagcattcgacgtcggacgagcactacggcagccggtcgggctcctgacagagatgatagcggaaatggagaagcagtggatgatcgtgaccaagtcgaatctgcaaatctgatgtcgaacgtggcagaaaatgccaacgaaatgcccaactccagcgatacgcgattcacctaccgtccccgagtccctgcacaccgacggatcaaagagtcccctctgtcttctggcgcaatctttaaacaatcgcatgcaggacttttcaatctgtgcattgttgttctcgtcgccgtcaattcgcgactcattatcgagaatctcatgaaatacggatggctgatccgatcgggtttttggttttctagccggagcctctctgactggcctctgttcatgtgttgccttacactgcccatcttcccccttgcagctttcgttgtggaaaaacttgtgcaaagaaactacatctcggaacctgttgtggtgtttctgcacgcaattatttctactacagccgtgctttaccccgtcatcgtgaatcttagatgcgattcggcttttctttctggtgttgctctcatgctctttgcctgcatcgtgtggctgaagctggtgtcttacgcacatacaaacaatgatatgcgtgccctcgccaaaagcgctgagaagggagatgtcgatccttcttacgacgttagcttcaaaagcctggcatatttcatggttgcacctacactctgctaccaacagtcttatcctagaacgcccgcagtgcggaagagctgggtggtgcgacagtttattaagcttatcgtgttcacaggactcatgggtttcatcattgagcaatacattaaccccatcgtccaaaactcgcagcacccccttaagggtaacctgctttatgccattgagcgtgtgctgaaactgagcgtccccaatctctatgtgtggctctgtatgttctattgtttttttcacctttggctcaacattcttgccgagcttctgcgattcggcgatcgtgagttttataaagactggtggaatgctaagacggttgaagaatactggcgtatgtggaatatgcccgtccacaagtggatggtgagacacatctactttccctgtctccgtaacggtattcctaagggcgttgcaatcgtcattgcatttctggtgtctgccgtcttccatgaactctgtattgctgtgccttgccatattttcaaactctgggcattcatcggtattatgttccaggtccctctggttcttatcacaaattatctgcaagataagtttagaagctctatggtcggaaacatgattttctggttcatcttctctattctcggacaacctatgtgtgtccttctctattatcatgacctgatgaatcggaaaggaaaggccgattaa

SEQ ID NO: 145 - DNA, 984 bp, Artificial Sequence
  codon optimised ORF
atgatgggagaggagatggaagagcgaaaagctacaggctatcgtgaattttctggacggcatgagtttccctccaatactatgcatgctcttcttgctatgggcatttggctcggtgcaattcactttaatgccctgctgctcctcttctcttttcttttcctccctttctcgaaattcctggtcgtctttggactccttctccttttcatgattcttcccatcgacccttattccaaatttggaagaagactgtcccgatacatctgcaaacatgcatgcagctactttcctattacactgcatgtggaggacatccatgcattccaccccgacagagcatatgtctttggatttgaaccccattctgtgctccccatcggagtggttgctctcgccgatcttaccggcttcatgcctctgcccaagatcaaagtccttgcatcttcggcagtcttctacacccctttcctgcgtcatatctggacttggctcggactgacacccgctacgaagaagaatttctcttctctgctcgacgcaggttatagctgtatcctggttcctggcggcgtccaggaaacttttcatatggaacccggctccgagattgctttccttcgggcacgtagaggcttcgtgcgtattgcaatggaaatgggttctcccctggtgcctgtcttttgttttggtcagtcgcacgtctataaatggtggaagcctggtggcaaattttaccttcaattttctcgagccatcaagtttacacctatcttcttttggggcattttcggtagccccctgccctaccaacatcccatgcatgttgtcgtgggtaaacccattgatgtcaagaagaacccccaacccattgttgaagaggtcatcgaagttcacgatcggtttgtcgaagctctgcaagacctctttgaaagacataaagcacaagtgggatttgccgatcttcccctgaagatcctgtaa

SEQ ID NO: 146 - DNA, 1431 bp, Artificial Sequence
  codon optimised ORF
atgggatcggaagctcaatataagaatgccatggcactgaacggtgaggaaggagagtattccgagcctgtcagccccactggtcagtatttcaactcttccgccctgtccattggtgttctcggcgtcctcgattcggaaattcccattgatgactctcccacaatgaaactccttgaagatgtctttctgcctattaatcctcggttttctagcgtcatggtgcaagatgagaacggcgctaaacagtggaaaaaggttgaggtgaaactggttgaccatgtcaacgtccctgtgtttcctcctggactttctcccgaatcctatgataattatctttcggactatctttctaaaattgcaacagaacaactgcctcaaaaccgacccctttggaatatccacattgttaaatacacaacttcgaacgctgccggatcgctcattttcaaacttcaccacgccctcggcgacggttattccctcatgggagcacttctttcgtgcctgcagcgagctgagaacccctccgttcctctcacgttccctgctctctccagcgcacccaatctttcggagaactctaactcggtttttcggagcctgtactctgctttcaacaccattagcgacttcggttggagccttcttaaatcctcgtttctggaggacgacagaacgatgattcgatcgggtgaccccggagttcagttcaagcccgttgttatttcgacaatgactttctcgcttgaccatattaagcaaattaagacgaaacttggtgtcactattaacgatgcaatcaccggcattatcttcttcggaaccagactctatatgcagggtacatccagcgataaactgtcgaacggtaactctaccgcacttgtgctgctcaatacacgaactatcggcggatataaatctgtgaaggatatggtcaaacctgacgccgattctccttggggtaatcagttcggcttcctgcatgtttccctgcctgagcttgcctccacggcagagtcttgtaaccctctggagttcatctggaaggcccagaagctgattcagagaaagcgaaattctaaggctgtgttcttcaccggacaactcctggagggcctccgaaagtacagaggccctgaagcaactgctaaatacatccattctacacttaagaactcctcgatgactatttcgaatctgatcggtcccgtcgaacgaatggcactcgcagaccatcccgttaaaagcctgtacttcatggttgtgggtgtgccccagagccttacgattacgatggtttcgtatatgggcaagcttacggtggctattggtacggagaaagacttcgtggacccccagaagttcaaatccagcgttgagaacgcattccaaatgatgctgaaagcagcccaagcaatcgcctaa

SEQ ID NO: 147 - DNA, 1458 bp, Artificial Sequence
  codon optimised ORF
atggcttgtcccggaactttctctgatgaaccccttacccctgctggtcgactgtttctgcagagccagacgaatatcattattcactgcatccttcggggaaagaatcccatcgacatcgacgcagttaagtcgactctgcgatcgtcccttatggtccgtcatcccagattttgctctcttctcgtccgggacaaaaacggtttcgaacactggcgaaaggcacaggttgacatcgaccagcatgtcatcgtcattgataaacgactcgataagtccgataatttcctcgacgaaagccgacgttttagctcggaggagggagaaggtgacgacgatgacgaggccgccgtcaaccagtatgtggccgacctgtctgtttcgtcgcctctttcgactgacaagcccctttgggagattcatattcttgttccccacaagtgtgctgtgtttagaatccaccacgcacttggtgatggtatctcgctgatgtcgatgcttatggcatcctgccgacgggctgatgacgcagatgcactgcctcgtatggtccctgagaaatctgccgagtttaaggatggcaaaggacgggattggttttggctgttcggcatcctttggggtttccttaaaatggtttgcttcacagtgatgttcgtgatggaatttgtggtgagctctctccttgtttgcgaccggaaaacggttattagcggcggcgaaggcgtcgagctctggcctcgaaaactggctacggcccggttccttctggaagatatgcgagtcgtgaagaaggcaattcctaacacaacgattaatgacgtgctcttcggtgtcgtctcctccggactttcgcggtatctggatcaccgtacccccaatgcccttcatgaaggacttcggatgacgggcgtcgcaatggtcaatcttcgaccccaaacaggctttcaggatcttagccaactcatgaagggagctgctgaagcacgttggggaaataagttcggactcattctccttcccgtttacttcaagaaggctggtaataaccctctggaatatgtcaagcgggcaaagcggatggtggaccgtaagaagcattccctggaagcttacttctcctatcgtatcggcgatctcgttatgtcccttctcggtagcaaatacgcctgtctcctcaattatcgtctcctgtgtaatacaactttcactatttctaatattatcggccctctcgaagaaattaccctcgctggtaatcctatcagcagcattaaggttaacacttcgtcgctgccccaggcaattacgatgcacatgctttcctacgccggtagagcagaaatgcaaattctggtggctaaggacatcgtccctgaccccgagttcctcgctaagtgttttgaagacgccctcctggagatgaaggaagcagtcatcggcaccgagaaagagtaa

SEQ ID NO: 148 - DNA, 1623 bp, Artificial Sequence
  codon optimised ORF
atgaatcgaaagtcggaagcaaaatgggtgcctaagcttttccaccagagcctgatctccatgaactccaccgagattcaggaaatcgaccaggttgaggctaagatcggaacggaggaagcctctcctgtgtcgaaggatgaggttcctctctcgaccggctctctgattttccacgagcccggtatcaattgttgcattatcgcaatcatgggatacaaaagcaagcttgatcccgcagtcatcaaagaaggtcttaaacagacgctgattaaacatccctgtttttccagcaaactcctgtctgctattgaccaaatgatgaactctggtaagaaaaagtggatcagaactcaggtcaatgtggacaatcatgtgatccttcccgaggtcgataccgttacagaatcgccctctcaattcatcgaggattatgtgacctatctgacaggcattcctatcgacctttccaaacctctttgggaactgcacctggtgaaccttaaaacccccgaggcagaagcagtcggcattttccgaatccatcattcgattggcgacggtatgagccttatttctcttcttctcgcctgctgtcgaaagtccagcgaccccaaggcactgcctacgctccccaaacaaaagcaagcagattcccgttacccccaccctcacggatttttccggctctttctggccatttggtctgttcttcggcttatcctgaacaccctcgtggacctccttctgtttgtggccacagttgttttcctgaaagacaccaaaactccccttaagggcagcagcggagtcgagcagaacgctaagaagatcgtccatcggactgtgtcgcttgacgacatcaaacttgtcaaggacgccatgggaatgacggtcaacgacgtcattcttggagtgacagaagccggactctcccgatatctcaatagaaaatacggtgaggttgatcgaggtaaagaggcagaacaaaagtctaatcaccttccccgtaacattcgacttagagccacggccctggtgaacattcgtcagaccgctggaatccaggtgtctttcctgtcgcctttttctcgggcctctgaaaaagtcgagtatgcatctcagagctttcttgttttcaagtcgaaggttaagtggggaaaccagattggctacatctgtatccccttcacgatcgcccttcggaatgaccctctcgactacctgcgaggtgctaaagcagcaggagatcgaaagaaactgtccctcgaagccatttgtacccatctgactaataaatgtgtggttaaacttttcggatcgaaactttccgccgcacttgtctaccgggttatctttaatacgacaatgacactgtccaatgtcgtcggacctgttgaagaaatcagcctgtatggccaccctatcgcctttattgctccttcggtctacggtcaccctcaggcacttacgtgccattttcagtcgtacatgaacaagatgagcattgtcctcgccgtcgatcctaatgtgatccctgatccccgtctgctttgcgatgatctcgaggaatcccttaaaatctttaaggatgccgtcgttctcgcaaaggacgcagattaa

SEQ ID NO: 149 - DNA, 1533 bp, Artificial Sequence
  codon optimised ORF
atggagagccggaacggattccgttggagaaaacagggcctcaaacccattgagacaaaaacaggcagcaaaggtgaagaagagaacaaatcctcctctggccaaagaaccgccgaggaggagcctctctctccttcggctcgtcttttccatgaacctaatttcaacgtgtacatcattgcaacccttggttgcaaaacgcgtatttaccctgatgtggtcaaggccaatctgggccatacccttctgaaacaccccagattcagcagccttcaggtcgaagacgaggataacaagggcgaaatgaagtgggtgcgaactgaagtggacctcgagcgacacgtggtcgttcccgacctggaccccaatatggaccttcctgataaatttctcgaggactacatctacaacctttccaagacgacgattgataagagccaacctctgtgggatctccatctgcttaaccttcggacctcgcagtctgaagctgttggcgtttttagaatccatcacagcctgggcgacggtacctcgctcatgagccttctcctcgcatgtacgcgacaaatgaatgatccccttgctcttcccaccatccctattagaaaaaagcaggaaagaaagaatgatcgtcgaggtttttggcggatcatgttcatgctttgttcggttttccaggtgttttggaactctgttgtggacgtttttatgttcatggcaactgcactccttcttaaagacacagagaaccctttcaagggactgcccggagtcgagtttactcctagaagaattgtttatggcacggtttcgctggacgacatcaagctcgttaagaacgctatgaacacaacagtcaatgatgtggcacttggtattacccaagctggtctgtctcgttacattaatagaatctacggcggcaataagaatgatggcgcagctattgaaatggataaccttcctaaatccattcgactgcgatctacgctcctggtgaacatccggcccagcgccggcattcaagccctcgccgacatgatggagaaagatgctgaagcaaaatggggcaactggatcggctacgtgcttctccccttcaccattgcaatccgggatgaccctcttgattacgtccgtgatgccaaagctacgattgatcggaagaaaagatcgctcgaagcaatttacactttctcgattgccgaactcgtgctgaaactgttcggcgttaaggccgcttcggctctctcccatagaattatgagccatacaacgatgtgtttcagcaatctggtcggtcccctcgaggagatcggattctatggccaccccatcgctttcctcgctccctcttcgtatggccaaccccatgcacttatgatcaatttccaatcgtatattgataagatgactatcgttctcagcgttgatgagggtaccattcccaatcctcaccaactttgcgatgatatcgtcgagtcgcttggccttattaaagacgcagtcgtcacgcgtggcctcgtctaa

SEQ ID NO: 150 - DNA, 1794 bp, Artificial Sequence
  codon optimised ORF
atggacatcatcggacttcggagccggagccacctgggcctgaaacagatcaaggtcactagagacatgaaggagggtcagtcggtgtccggagaagaagaagagcctctgagccccatggcccgaatgttccacgagcccgactctaatgtcaaggaaatcaacgacgcactgcacgccattcctatgatcctgcaaacactcacgtgtagcaacatgaactctgcctatagacagggtagacgtgtcgcagacgaaaacaacggtggagagcttaaatgggtcaagacggaggtcgaacttgaaaagcatgttattatcccccgtgttgatgaggagatggcaagccaaggcgccgctgacaagttcgtcgaagattatatcgctaacatctccaagactaaaatttcgctctccatccccatgtgggattgtcatattcttaacatcaaaacatccgatgccgaatctgtcctggttctgctggttcatcactcgctgggagatggaacttccctgatgtccctccttatctcctgttcccggaagctgtcggatcttctcgccctcctcactttccccgctatgaagaagaagcagatgcctaccaccacgacaactacttggctttgtttctggatccggctctggagcttctttcttcttatttggaatacgtcggttgacctcctggtgtgtgttgctactctctttttctacaaggacacgcccacgcccctcaagcctagctctagacgagtggcttgtacccctggtcgaattatgcgacgaacgttcactcttgacgacatcaagcttgtgaagaacgccacgaatatgactgtcaacgacgtcgtgctggctatcacgcaggctggtctttcccgttaccttaaccggaaatatggaaagacgaagcgtaatgaggccggtcgaaagtgggaagataacctccctaacaatattcggctccgtgctacacttttcattaacctcagatcctcgcctggcatttatgcacttggtgagatgctcaagaagaactctaaggctgaatggggcaataaaattggttacgtgcttttcccctttaccatcagcctgaaggataatctccttgactacatccgggacgtcaaagcagccactgatcgtaagaaagcctaccttgaagcaaagttccgtcttctgatggccatggtcttcgtcagattttaccggacaaaattcgccaaattcccttctacaacaatgtggttttcgaacgtggcacgtcctcaggatgacatcaccattttcggaaatcaagtcgcctacattgctccctctctgtacggtcagcttgttgctctgacagtccacgtggtttcgtacgctaaaatgatctctatggtcctctctgtggatgataacatcatctcggacccctaccaactctgtgacgaccttgaagagtcgcttaaactgattaaaacgtcggtggtgtcgcctcttgtccgagtccgactttcctacagcagaactaagtcccggtccacgcttgtgatcctggctaagttcccctcgaccacgatttggtttagcaatgtggcaagaccccaggatgagattacaattttcggcaatcaggtcgcctatattgcaagcagcgtctacggtcaacctaaggcacttactattcatgtggttagcttcgctaaaaaaatctcccaacctgttgttctgcttgccggacatcccgtgcctctgacacccatgcttcctcggcgagctggtgccgacgctagcctttaa

SEQ ID NO: 151 - DNA, 1545 bp, Artificial Sequence
  codon optimised ORF
atggatattattgacctgcggagcggatctcaccttggactcaagcagattaaagtcacaagagagatggaagagggtcgaagcgtttctggcgaagataatgaacccctgtcgcctatggctcgaatgttccatgagcccgattccaacgtttacattattactattgtcggatttaaaaaccctattgagcccaactcctttaaagcaaaccttgttcacactctgctgaagcatcccagattctcgtcggttcaagtcgccgacgagaacaacggaggagagctcaagtgggttcaaaccgaggttgagctggaaaagcatgtgatcgtccctaaggttgatgaagagatggcatcgcaaggcgccgcagataaattcatcgaggattacatttccaatatgtccaagactaagatttccatgtctatccctatgtgggactgccacatccttaatctgaagacgtccgacgctgagtcggtcctcgttctgcgtgttcatcattcgctcggagacggaacatctctcatgtccctgcttatctcctgttcgagaaaactttttgaccctctggcactgcccacgtttcctgccatgaaaaagaagcctattgctacaacgacatggctgtgcttctggatcaaactgtggtcctttttccttcttatctggaacacgctggttgatatgcttatgtgcgttgctacactctatttctataaagacacgcctacacccctcaaacctcctagccggtcggttgcatgtacgcctaaaagaattgtccgaagaactttttccctcgatgatgtgaaacttgttaagaatgccacaaatactctgatctggcttattagagtttaccttaaatgttttgctattggccctttcgtggaccgacagcgatgtcggccctccaatcattcttccagactgatctccctggacatttctacggtcaatatgcggcacgaggcaggtaaagaatgggaagataaccttcccaacaatattagactcagagccacgctttttatcaatctgcgtagcagccctggaatctacgccctcggtgagatgctcaaaaaaaattcgaaagccgagtggggaaacaagatcggctatgtgctttttccttttactatcgcccttaaggataatcctctcgagtatatccgagacgttaaagaggctatggatagaaagaaggcatctctcgaggctaaatttagacatctgatggcaaccgtgttcgtgcggttttaccgtacgagactcgcaaagttcccctccactacgatgtggttctccaacgtggctggtcctcaggacgaaatcaccattttcggaaatcaggttacttacatcgccccctcgctgtatggtcagcctgttgcccttactatccatgtcgtctcgtatgccaagaaaatgtctatggtcctcagcgtggacgataacatcattcctgatccttatcagctttgcgatgatctcgaggagtcgctgaagctcattaaaaaatctgtcatttcgcaataa

SEQ ID NO: 152 - DNA, 2013 bp, Artificial Sequence
  codon optimised ORF
atgtcttttctgcgacgtagaaaagtcaccgattcgagcaaatctcaaaactccgactctaacgccggtaaggaagacgacaagaagaagcagtataaatcccccaagcgagagtcctatagctccaaaaaatggagctgctgggactcctgttgctggtttattggtctgatttgctccatgtggtggtttcttctttttctttataacgctatgcccgcttctattccccaatacgttacggaagcaatcacaggccccctgcctgatccccccggagttaagcttcgtaaagaaggactgacagtcaaccatcccgttgtcttcgtgcccggcatcgtcactggcggcctcgagctctgggaaggccatcactgtgctgacggccttttccggaagcgactttggggtggatctttcggtgaactttataaacgtcccctctgctgggctgagcacatgtccctcgacaacgaaacgggactcgaccctcccggtattcgagtgcgacctgtgtccggcctggtcgcagccgactactttgccgcaggctactttgtctgggccgtccttatcgccaaccttgcacaaatcggttacgaggaaaaaactatgtacatggccgcatacgattggcgactcagcttccaaaatacagaagtgcgggaccagactctctctcggatcaagtctaatattgagctgatggtcgctacgaatggtggaaagaaggtggtggttattcctcactccatgggtgttctctactttctccactttatgaaatgggttgaagctcctgtccccatgggtggcggaggtggctcggattggtgtgcaaaacatatcaaggccgtgatgaatattggagcccctttcctgggcgttcctaagtctgtttccggactctttagcattgaggcacgggatattgcaattgctcgagcatttgcacctggcttccttgacaaggacgtttttggtctgcagacgttccaacatctcatgcggatgacccgaacttgggacagcaccatgtcgatgatccctaagggaggagacacgatctggggcggcctggattggtcccccgagggaggctcctttaactgctctgcaaagaaactcaaaaataacggaacccgaactggacataatgccaacagcaacctgggtaatatgaagtcggtcaattatggtcggattatctcgttcggcaaggacgtggctgaagcacattcttccaaaatcgaaagagtcgattttcgagacgtcatgaagggaggcaagctggcaaatagctctaactgtgatatctggacagaatatcacgaaatgggtaacggagccattaaggctgtggcagattataaagtgtacacggctggctctatccttgatctgctgcattttgtggcccctaaacttatggctcgtggaggcgcacatttctcgtatggtatcgcagacaatctggacgaccccaagtacgaacactataagtattggtctaaccccctggaaaccaagctgcctagcgctcccgatatggagatttattcgatgtacggagttggaatccccacagagagagcctacgtctataaactcacaacggctactgattgctatatcccttttcagattgacacttctgcagaaggaggatcggaaggttcgtgtcttaagggaggagtcttttcggtcgatggtgacgagactgtccccgtcctgtctgccggctttatgtgtgcaaagggctggcgaggaaagacgcgattcaatccctcgggtatccgaacttatatccgagaatacaatcatgctcctccttctaacctcctcgaaggtcggggaacacagtcgggcgcccatgtggacatcatgggtaactttgccctgattgaggacatcattcgggtcgccgctggcgccacgggtcaagatctgggcggcgatagagtctattcggacatcttcaagtggtcggaaagaattaaccttcaactgtaa

SEQ ID NO: 153 - DNA, 2076 bp, Artificial Sequence
  codon optimised ORF
atggcttcgattcttagatttcggaagctgtgctacgtcgaacaggctgtgaaatgtgcttcggtcggctttgaatcctttgaccagtcgcccaaaattgaccaaaagctcgacaagaaggaggaggaagtcattagcgcaaataatttcgcacttgagattatcaaaaagcggaaacaacctcggaagcaacctaaggagtggcggcgactggattcgtgttgctggatgatcggctatctgtgttctacgtggtggctccttctgttcctttatcactctcttcctgttacgctccttcaagtccccgaatctcccggtgttcggcttaaacgagagggactcgcagcccttcaccctgtggtccttgtccccggcatcgtcacgggtggccttgaactctgggagggtcaaccctgcgcagatggcctgtttcgtaagcggctttggggtggcagctttactgagatctttaagcgacccctgtgctggcttgaacacctgtctctccactcggagacaggtcttgacccccctggtattcgggtgcgtgccgttcccggactggtcgctgcagactacttcgcacctggctactttgtctgggccgtcctcattgagaacctggctaagatcggctacgagggtaaaaacctgcacatggcagcatacgactggagactttcgtttcaaaacaccgaaatccgggatcacgctctgagccgactcaaaagcaaaattgagcttatgtatcttacgaacggctataagaaagtcgcagtggtcccccactcgatgggcgtcatctattttctccattttctgaagtgggtggagactcctccccccatcggcggcggcggtggacccggctggtgcgctaaacatattaaggcaattatgaatattggccctgctttcctgggcgttcctaaagctgtgtccaacattttctccgctgaaggtaaggatattgcctacatccgtgctatggctcccggtcttctggacagcaaaattctgggcctccaaacgctggagcatgtcatgcgtgtttctcggacctgggactccattgtcagcctggtccctaagggaggtgagacgatttggggcaacatggactggagccctgaagagcgtcatgcatgcgatttttctcgtaagcggcactcccagcccagccccattgataataatgttaacaattctgacgtgaagcgaggcttcagagtgaaggaccccgtccagtatggacggattattagcttcggaaagacggcctctcagctgcatagctcccaacttcccactatcgactctaaggaatttctccggaccagcgcctcgcaaaatctgaacttctcctgcggagaagcatggaccgaatacgacgagatgagccgggagggcattcaaaaagtcgctgcaaataaagcctacaccacccaaactctgcttgatctgctccgtttcgttgcccctaagatgatgcttcgagccgaggcccattttagccatggcatcgctgacgacctggacaaccctaagtataatcattataaatattggtcgaatcccctcgagatgaaactgcccgatgcccccgatatggagatctactgtctttacggcgtgggcatccctaccgagcggagctatgtgtataagctttcccccacgtctggctgcaaatccatcccttaccagattgatagctctgttaatggagaggacggatcttgtctcaagggtggcgtgtacttcgctgacggagacgaaaacgttcccgtgcttagcgctggtttcatgtgcgctaaaggttggcggggacggacacgatttaaccctagcggaatcgctacctatattcgagaataccgtcataaacccccctcgtctctccttgagggacgtggaatcgagtccggtgctcacgtcgacatcatgggcaatgtggccctcatcgaggacattctccgaattgcttctggcgctaccggtaaagaaattcgtggcgaccgaatttactccgacattctcagaatgagcgaacgtattaaccttagactttaa

SEQ ID NO: 154 - DNA, 2058 bp, Artificial Sequence
  codon optimised ORF
atgagcgccatccgtagaagaaaacctatcaacgagtcggacaaaactagcgaagcctcggattcgaaaccccacaaagagaaggaggaggaacagcacggtgacgatgatggctcggacaaagataaaaaaaaaatccccagcaagatcaaaaagaaacacgaagaaaagcaacccaaatggtcctgcctcgattcctgctgctggttcattggatgtatttgcgtcacgtggtggctcctcctctttctctacaacgctatgcctgcttcctttccccaatacgtgactgaggcaattactggcccccttcccgacccccctggcgttaagctcaagaaggaaggtctgaaagcaaagcatcccgttgtgttcgttcctggcattgtgacgggtggcctggagctctgggagggaagaaaatgtgcagaaggactctttcggaagcgtttctggggtggtaccttcggtgaagtgtataagcgacccctctgttgggtggaacacatgagcctggataatgagacgggtcttgacccttgtggcatccgggtcagacccgtgtccggactggttgctgccgattattttgcccctggatactttgtctgggccgtgctcattgcaaacctggctagaattggatacgaagagaagacaatgtatatggctgcctatgattggcggctctccttccagaacacagaagcacgagatcaaacactcagccgtattaaatcgaacatcgaacttatcgtggccaccaacggcggaaaaaaggcagttgtcatcccccattccatgggcgtcctctattttctccatttcatgaaatgggtggaggcccctgcccctatgggaggaggcggtggacctgactggtgctccaagcacattaaggctgtggtcaacattggaggtcctttcctgggcgtccctaaggcaattgccggacttttcagcgccgaagctaaggatattgccgtggttcgtgcaatcgcacccggatttctcgacaatgacatttttcagctccaaacccttcaacacgttatgcggatgtcccgatcgtgggattctaccatgtctatgatcccccgaggaggtgacacgatctggggcggcctggattggtcccctgaggagggttacagctgcgccaaaaaacgtgagcgaaaaaatgacacacagatcgcagaggaggccggcgtggaatcggctgtgtcccagacacggtctgcaaaatatggaagaatcatttctttcggcaaagatgtggctgaggcaccttcgtctgacgtcgagagaattgacttcagagacgctgttaaaggccactcggcagcaaacacgacctgtcgtgatgtctggacggaataccatgacatgggtttcggcggtatcaatgctgtcgctgaatacaaaacgtacacagccgagagcattgtggaccttctccacttcgtggcccccaagatgatggctcgtggaactgcccatttttcttacggcatcgccgacaatctggatgatcccaagtacaagcactataaatattggagcaaccctctcgagactaagcttcctaacgcccccgaaatggaaatcttttccctttatggtgtgggcctccccaccgaaagagcatacatttacaaactgtcccccgctgcagagtgctacatcccctttcaaattgatacctcggcagacgatgaagagacatgcctgaaggacggagtgtatagcgtcgacggcgatgagactgtccctgttctgtcggctggcttcatgtgcgctaaaggttggcgtggaaagactcggtttaatccctctggtatccggacttacatccgtgaatactcccactcgccccctgctaacctcctcgaaggccgaggaactctgtccggcgcccatgtggacattatgggaaattttgcactgatcgaagacgtgatcagagtcgccgccggagcttcgggtgaagagcttggcggcgatcaagtgtactctaacatctttaactggtcggaaaaaatcgatcttcagctttaa

SEQ ID NO: 155 - DNA, 1503 bp, Artificial Sequence
  codon optimised ORF
atggcccctgctaaatccggcagatcgtttccctctatcacgaaatgcgagggtagcacctatgagagcattgccgctgatctggacggcacactgctcatcagccgatcttcgtttccttactttatgctcgtggcagtggaggcaggatctctgctccgaggactcattcttctgctgtctcttcctctcgttattgtttcctatcttttcatttccgaggcaattggtatccagatcctgatctttatcagcttcgctggcctgaagatcagagacatcgagctcgttagcagagctgttctgccccgtttttatgccgctaacgtccgaaaggaaagctttgaagtgttcgatcggtgcaaacgtaaagtggttgtgacagcaaatcccacatttatggtggaaccctttgttaaggacttccttggcggcgacaaagtgctcggtacggagatcgaagtgaaccccaaaacaaaaaaggctactggatttgtgaagaaacccggcgttcttgtgtcggagctgaaacggctggccatcctgaaggaatttggtgaggaatctcccgacctcggcattggcgatcgtgagtctgatcatgatttcatgtcgatctgtaaagaaggctacatggtccacccttccaagagcgccactcccgtgcccctggatagacttaaatctcggatcattttccatgacggccgttttgttcagcgtcctgaccccctgaacgcccttatcacctacctttggcttcctttcggttttattctctctatcgtccgtgtctactttaatcttcccctgcctgagcgtattgtgagatacacgtacgagatgctcggcatccaccttgtcattagaggaaagcgtccccctcctccctctcccggtacacccggaaatctttatgtttgtaaccaccgttccgccctcgaccccattgttatcgcaattgctcttggtcggaaggtttcgtgcgttacctattctgtctcgcgactgtcgagatttctctcccctattcccgcagtcgccctgactcgagatcgtgccgccgacgccgccagaattagcgagcttctccaaaaaggtgatctggtcgtctgccctgaaggtactacatgccgagaacagttcctgcttcgttttagcgccctgtttgcagagctgtcggatcgaatcgtgcctgtggccgtcaactgccgacagaatatgttttatggtacgacggtgcgtggtgtcaaattttgggacccttatttcttcttcatgaaccctagacctacttatgaagtgacgttcctggatcgtctgcctgaggaaatgacggtcaaagctggcggaaaatcgtcgattgaagttgcaaaccatgtccaaaaggtgcttggcgacgtgctgggcttcgagtgtacgggcctcactcgaaaggacaagtacctcctcctcggtggtaacgacggaaaggtcgagtccatgtacaacgcaaaaaagtaa

SEQ ID NO: 156 - DNA, 1131 bp, Artificial Sequence
  codon optimised ORF
atgagctcccgtggcggtaagctgtcttcgtcctcgtccgagctcgatctggacggacccaacatcgaggattatctcccttcgggttcttcgatcaacgaacctcgtggcaagctgcggcttcgtgacctgcttgacatctctcccacgctgacggaggctgccggtgcaattgtcgacgattcttttacccgttgtttcaagtcgaatcctcccgagccctggaactggaacgtttacctcttccccctctggtgtttcggagttgccgtccggtatctcattcttttccctgcccgtgtcgttgttcttactatcggttggatcatctttctcagcagcttcatccctgtccacttccttctcaaaggtcacgataagctcagaaaaaaaatggagcgtgtcctcgttgaactgatgtgctccttttttgtggctagctggacaggagttgtcaaatatcacggaccccgaccctctattcgtcctaaacaggtgtttgttgcaaatcataccagcatgatcgattttatcattcttgagcagatgacagcatttgccgtcattatgcagaagcatcctggatgggtcggactgctgcaaagcaccattctggaatctgttggatgtatttggttcaacagatcggaagccaaggatagagagatcgtggctaagaaacttagagatcacgtccaaggagtcgacaacaaccccctgctcatcttccctgagggaacctgcattaacaaccagtattcggtgatgtttaaaaagggcgcctttgagcttggctgtacagtgtgccccatcgcaattaagtacaacaagatttttgttgatgccttctggaactctagaaagcagtcgttcacgatgcaccttcttcaactcatgacatcctgggccgtcgtctgtgacgtttggtatctggagcctcagaaccttcgacccggcgaaactcccatcgaatttgccgaacgggtcagagacattatttctgttcgagccggcctcaagaaggttccttgggacggttaccttaaatattccagaccctcgcccaaacatcgggagcgaaaacaacaatccttcgctgagtctgtgctgcttcgacttgaggaaaaataa

SEQ ID NO: 157 - DNA, 1623 bp, Artificial Sequence
  codon optimised ORF
atggtgttccccgtggtgttcctcaaacttgctgactgggttctttaccaactccttgcaaactcgtgttatcgagcagcccgtaaaatgagaaattatggctttttcctgagaaatcaaacgctgagatctcccccccaacagcaagccgcatccctttttccttccgttacaaagtgtgacgtgggaaactctcgtagattcgatacacttgtctgcgacattcacggcgtgctcctgggctccgacacatttttcccttatttcatgctcgtggctttcgagggtggttccattgtgcgggctttccttctcctcctttcgtgtagctttctctgggttcttgactccgagctgaagctgagaatcatgatcttcatttccttttgcggtcttcggaagaaggacattgagtctgtgggccgagccgtgcttcctaagttctatcttgagaaccttaatctccaggtgtatgaagtttggtcgaaaacttctagccgtgtcgtgttcacaagcattcctcgagttatggtggagggttttctccatgagtatatgtccgcatcgggcgtcgttggtactgagctccacacagtcggaaaccgatttactggcctcctctcctcttcgggccttctcgtcaaacataacgcactcaaggaacattttggcgacaaaaagcctgatgtgggactcggttcttctagcctccacgatcagtactttatctccctgtgcaaggaggcttacgtggttaacatggaggacggtaaatccaacctctcctcctttatgcccagagacaaataccccaaacctctgatttttcacgacggccgactcgcattccttcctacaccctttgctaccctgagcatgttcctttggcttccttttggaattgtcctctccattcttcgtattttcgtcggaatctgtctgccttataaactcgccgtgatctgtgcaacactttctggtgtgcagctcaagtttcaaggttgctttccctccagcaacagccagcataagaaaggcgtcctgtacgtctgtacccacagaacacttctggatcccgtgttcctgtcgacagccctttgtaagcctctgaccgcagtgacgtattcgctctcgaagatgtccgaactgatcgcacctatcaagacagttagacttacacgggatcggaaacaggacggagagactatgcagaagcttctttcggaaggcgaccttgtggtgtgccctgagggtacaacctgccgggaaccttatctcctgcgtttctccagcctcttcgctgagctggccgatgagatcgtccctgtcgcaatcaatgcccacgtgtccatgttctacggaactacggcttctggcctcaaatgtctcgatcccattttcttcctgatgaatccccggccctcctatcacgttcaaatcctgggaaaagttccccaagaatttacgtgcgctggaggccggtcgtctctggaagtggccaactacatccagcggaagcttgccgatgcacttggatttgagtgtacgacccttactcggcgagataagtatcttatgcttgcaggaaatgagggagtcgtccacgagaacaagagaaactaa

SEQ ID NO: 158 - DNA, 1614 bp, Artificial Sequence
  codon optimised ORF
atggccaaacttagcatggagttctcgttcttccaaacactgttcttcctcttctgtagagttgtctttcgacagtcgaaaaaccacaaatctcttcatagaaacgtcagcaacattcacgcaaatgaaggcaaataccataaatatccttcgttcgttcaccggagcaacctctcgaaccaaactctcgttttctccgttgaggaagccctgctcaagtcgtcttccctctttccttacttcatgctggtcgcctttgaagctggtggccttctccgggcattcattctctttgtcctgtaccccattctttgtctggtttctgaagaaatgggacttaagattatggtcctggtgtgcttcttcggtattaagaaaaaatcgttccgtgtgggttctgccgttcttcccaagttctttcttgaagatgtcggccttgagccctttgagatgctgaagaaaggcggaaaaaaggttgcagtgtctaaaatcccccaagtgatgatcgagagctttcttaaagactatcttgaaattgacttcgtggttggtcgagagctgaaggagttctgcggatacttcctgggtgttatggaagagaaaaaacggtcgaaggctgctcttgatgagattatcggttcggaaagcatgggttttgatgtgattggtatctccggactcaagaagtcgcttgactatcacttttttagccactgcaaagaaatctatcaagtccgtaaggcagataagcgaaactggcgacatgtgcccagacaagagtactccaagcccctgatttttcacgatggacgactcgccctgcgacccactctcgttgcctcgctcactatgtttgtttggtttcctttcggcctcgctctgtctattctgagagctgtggtcggcctgatgctgccttacaagatttcgatccctctcctcgcttattcgggcctccatctgttcctctcgacacctgaaagcagcctccatcctctttctctgcccaattctaaaaaacagaaccctaaaggaagactgtacgtgtgtaaccaccgtaccctgctggatcccgtgtatctttcttttgcccttcagaaggatctcactgcagtcacctattccctgtctcggatttccgaactcctcgcacccattaaaacggtccgtctcgcccgtgaccgagatcaagatggcaagatgatggaaaagatgcttaacctgggcgaccttgtcgtgtgccccgagggaacaacgtgccgggagccttaccttcttagattttcgcctctcttttctgagatgtctgatgacatcgtgcctgtggcaatggactccaatgtgtcccttttctacggaaccacggcatctggtctgaagtgtctcgatcctctctttttcctgatgaatcctcggcccatctacacggtgcaaatccttgatggagtgtcgggactctatacctgccatgacggccaacgaagccgatttaaagtggccaaccaagtccagaacgaaattggtaaagcactcggtttcgaatgcacgaaactgactcgacgagataaatacctcatcatggctggtaacgagggtatcatttcgcagacgtaa

SEQ ID NO: 159 - DNA, 1467 bp, Artificial Sequence
  codon optimised ORF
atggagtccgttgtcagcgaactggaaggtgtgcttcttaaagatcctgaccctttttgttattttatgctggtggcctttgaagcatcgggacttattcgatttgctcttctccttcttttctggccccttattcgtctccttgacatgctgggacttgatgatgctggacttaagctgatgacgttcgtcgctacagccggactgcgagaggcagagattgaatctgtctctagagcagtgctgcctaagttctacatggatgacgtggacatggaggcttggaaggttttctcctcttatgataaacgagtggtggtgacgaagactcccagaatcatggttgcaagattcgttaaagagcacctccgggcagacgaagtgctgggttcggaactggtcgtcaatcggttcggatttgctactggatttgtgaagggtgatattgcctccatttcgtcgcgtgtggccaagctcttcgttgatgaagaacctaccctgggcctgggacgagcaccctcgtccttccaattcctgtcgctctgtaaaaaacagatgcaccctccccttatgactaatcaaaacctccatgatcaccagcttctgcggcccctgcccgtcattttccatgatggacgtcttgttaaaagacctactccttcgactgccctcctcattcttctttggatgcccctcggcattcttctggctacaattcgaattgtggtcggtcttattctccccatgcgagtcattccttacatgtctcgactgtttggtggtaaaatcattgtgaagggtaagcagcctcctcctgtttctggctcctcctcgggtgttctgtttgtctgtacacatcgaacgctgatggatcccgttgtgctttccgctgttcttatgcgaaagattcccgctgtgacgtactccatttctcggctcagcgagattctctcgcctattcccacggtccgacttactcggatcagagaggttgacgcagagaaaattaagcgtgagctggcaaaaggtgatcttgtggtttgccccgagggtacaacgtgccgagagccctttctccttcggttcagcgcccttttcgcagagctgaccgaccggatcgtgcctgtggctatgaactaccgggtgggcttctttcacgctacaactgctcgtggctggaagggactcgatcccatttttttttttatgaaccctcgtcccgtctacgaggttacatttctgaaccagctgcctgctgaagctacttgtagctctggcaaatctcctcacgatgttgcaaactatgttcaacgtatccttgcagccacccttggtttcgaatgtacgaatttcactcgaaaagacaagtatcgtgttctcgcaggcaacgatggaacggtttcctgcacttctttcgtcgatcaggtgaagaaggtcgtctcgactttcaagcccttctttcaataa

SEQ ID NO: 160 - DNA, 1782 bp, Artificial Sequence
  codon optimised ORF
atgaaactgtctagacgacaaactgttcttaccaatgacaacaacaactgcaacctcaaactcgcagcttggccttgttcgtttggatataagagacggaaagcccggcagacatccgcaatccagcctaagtatcaagttcccaccgcctcgtactcttctgccaccgcccaacggtcgaaagcatctcccagactcatccactggctcgccgtgaaggattcgtggaagtctcgagcccttaacgttcttattctgctgttcctcaacatggccatcaatgctttccctactgtgcagaactgtgcctcgatcggccgagaaaaacacaccatggttgctgatatggacggaacactcctcatcagccgtagcagcttcccttacttcgctcttgttgcatttgaggtcggcggagttctgagactgttcttccttctccttgcatcccctctggcaggccttctgtattactttgtctcggagtcggctggaatccaagtccttatcttcgccacctatgtcggcatgaaggttagcgacattgaatccgtggctcgtgctgttctccctaaattctacagctcggatcttcatcctgagtcttggcgggttttttccagctgtggaaagagatgtgttcttaccgctaaccccagaatcatggtcgagccttttcttaaagattttctgggcgcagacatggttcttggtactgaaatcggcatttacagaggccgtgctactggattcgtctgtcagcccggagtccttgttggcaaaaacaaagctgacgcactccaaaaagccttcggcgaggctcttcccgatgtcggactcggcgaccgacatactgatattccctttatcgctctgtgtaaggaaggatacatcgttccccctaagcctgaggcaaaagccgtcagatcggagaagcttcccaagcctgtcatttttcacgacggccggcttgtgcagaaacctacacctctgatggccctccttattattatttggattcctatcggattcctgctggcctgccttagaattaccgccggcagcctcctccctatgcctctcgtctattacgctttctgggctctcggagttagagttactgtgaagggcactcctccccctcccgtcaagaagtccaccggccagtccggcattctgtttatctgttctcaccgtacactgcttgatcctatcttcctcagcacagcactgggccgacccattcctgttgtgacctactccgtttccagactttctgagatcatcagccctattaaaaccgtctccctttctcgtgatcgggccactgatgcatcgatgattaaaaagcttcttcaggagggtgaccttgccatttgtcccgagggaacaacctgccgggagccctttctgctccggttctcggcactcttcgctgagcttacagaccaacacgttcctgtcgcaatggtgaatagaatgtcgatgttccatggaactaccgccagaggctggaagggcatggatcccttttacttttttatgaatcccagccccgcctatgaggtgacattcctcaacaaacttcccatggaactcacctgctcgtccggaaaatcctcgcatgagatcgcaaactacattcaacgtgtcattgctgctactcttagctatgagtgtacgtctttcactcgtaaggataagtatagagctctggcaggcaacgacggaacggttgtcgaaaaacctaaattcagcgccaacaaagtgatgggttgttaa

SEQ ID NO: 161 - DNA, 1317 bp, Artificial Sequence
  codon optimised ORF
atgggtagccgtattcttccctctgcctcgctttttctctgtagcgtctttctttcgagcttttgtgctagctgccgatcttccctcagcattcctccccttcgggccagctttccttgcgcctctagcaagtctcttgcccccatttgccggctccctctgcctcatgcctgtggactcggtatcctcgtccttttcactgctagctcgctcgccatggctgatgtcgtggcagatgtggtcgcccccgtcaagcccaaaaatcgtgcatccaaaaaatttagctcccgtggagttgacctcgacgtgctgcttgagatgtcttctagcaactcctctcccctcctgtcgcccaaagtgttcctccgtgacctcaagcgaagacctcttgctctcaccaagaagattctttcctgggcactggaaggtgccagaagcacagcattctctctgggtaccggtggtaaatttcactatcccgcccgtcgggaatttttcgcaagcggccggctcctctttcttctgcttgccagccctctcgcaggtacgctgtactatttcacgagcgagtcccatgacatccgtgtgcttatttttgcaacctttgtgggaatgaaagtgtcggacattgaatcggtcgcccgggcagtcctccccaagttctacagctccgacctgcaccctggatttcgattcatgtgggaggagatgtgcgcctatggtgattcggagggccattctggcgtgaagtctcccggtgtcctcgttggaaagaacaaagcagatgcactcgaaaatgagtcccatgtggtgcctgctaaagcagaggcaggtgcagtttcgcatgacaaactccctaaacccatcgttttccatgatggccgtcttgtccaaaatcctacccccttcatggctctccttacgatcctgtggatccctgtgggctttctgcttgcctgtcttagaattgccgtgggcatcctgctccccacccctctggtctattacgctatgtggcctcttggatttagaatccatattaagggcacccctccccaacctacaaaaaagtctatcggtcagacggtcgtcctgttcgcctgctcgcatagaacactcctggaccctattgctctcagcgtcgcactttgcagacctattcccacagccacgtattccattagcggactctcggagatcatctcgcctatcaaaaccgcacggctctgtctggatcgtgctactgacgcaagcattatcaagaaacttctcgaaaagggtgatctcgtcatctgccccgagggtactaaaacataa

SEQ ID NO: 162 - DNA, 1524 bp, Artificial Sequence
  codon optimised ORF
atggctaagaccaaggaaaacccctttcctagcatcgcccaatgtgcttcgatcggtcgagacaagcatacagtcgttgctgatatggacggcaccctgctgagaggaagaagcagcttcccctactttgctcttgtcgcatttgaggtcggtggcatcctgcggctgctctttcttctgctggcttcccccgtggctggcatcctgtactactttatctccgaggccgcaggcattcgagtcctcatttttgctacctttgtcggtatgaaggttagcgatatcgaatctgtcgccagagcagttcttcctaagttttattctagcgatctgcaccctgaaacctggagagtcttttcttcgtgcggtaaaagctgcgttctcacggctaatcctcgagtcatggtggaagcatttcttaaggattaccttggcgcagacatggtgaccggcaccgagattcatgttttccgaggccgagcaaccggcctggttaaatcccctggtattctggtgggtaaaaacaaggttgatgctctggaaaatgcatttaaggataagcctgtggctgacatcgcactcggagacagaaagaccgattatcctttcatgaagctctgtaaggagtcgtatgtggttcctgccaaacccaaagttgaggccgtgagccacgacaaactccccaaacccattgtttttcatgacggtcggcttgttcaaaagcccaccccttttatggcacttctgaccattctttggattcccgtcggtttccttctcgcctgcctgcgaatcgccgcaggagctcttctccccatgcccctggtctactacgctttctgggctctgggagtccgtgtgcatattaagggaactcctccccctcctgcaaaaaaatctattggccagaccggtgtgctttttatttgctcgcatcgtactctcctcgaccccattttcctttctactgccctgggcagaaagatccccgctgtgacctactcgctttcccgtctcagcgaagtgatttctcctatcaaaactattagactttctcgagatcgggctacagacgcctcgatgattaagaaacttctggaggcaggagatctggtcatttgccccgagggcaccacgtgtcgggaacctttcctccttagattctctgccctgttcgcagaactcactaacgagctcgtccctgttgcaatggccaacagaatgtctatgttccatggtactacggcccgtggttggaaaggaatggaccctttctacttcttcatgaatcctagccctgcttacgaagtcaccttcctgaacaagctgcctcatgagcttacttgttctgctggaaagtcttctcatgaggtcgccaactatatccaacggatgatcgccgcatctctgagctatgagtgtacttcgtttacacggaaagacaagtaccgtgctctggctggcaacgacggtaccgtcgttgagaagcctaagctgtcgcccaataaggttatgggttgctaa

SEQ ID NO: 163 - DNA, 1512 bp, Artificial Sequence
  codon optimised ORF
atggttatgggcgcacaccgacggtttgagcccatttcgaagtgctcgaacgagggccgatcgaatcaaacggttgccgctgacctcgatggcacgctcctcctctctcggtccgctttcccttatttcatgctcgtggctctcgaagctggttctcttgtgagagcccttgtcctccttgctagcgttcccttcgtgtacttcacctatctgttcatttctgagtccgtggctattaacacgtttattttcatcacatttgcaggcctgaaggtcaaagatatcgaactcgtctcgcgttctgtcctccctaagttttatgctgaggacgtgcatcctgagagctggcgagttttttcttcgttcggcaaaagatatatcatcacggcatctccccggattatggttgagccttttgttaagacctttcttggtgcagataaagttattggtacggaactggaggtcaccaaatcgggtcgagctacaggttttactattaagcccggcgtgctcgttggagagcacaagcgagccgctatcagcaaggagttcggtatgaagctgcccgatctcggcctcggtgatagagagactgaccatgacttcatgagcctttgcaaagagggatatatggtgcctcgatctaagtgcgacccccttccccgtaacaagctgctctcccccatcatttttcacgagggacgtctcgtccaaagacctacccccgtggctgctctgctgacattcctctggctgcctatcggttttatcctcagcctccttcgagtttatcttaatatccccctgcctgaacgtattgcacgttacaactataaaatcctgggtatcaaacttattgtcaaaggcaaccctcctcctgcacctaaaaagggtcaatcgggagttctcttcgtgtgcaaccacagaacagtcctcgatcctgtcgtgacagcagttgccctcggtcgaaaaatctcctgcgttacctattcgattagcaagttcactgagatcatctcgcccatcagagctgtcgccctcagccgggagcgagaaaaagacgccgccaatattaagagacttctcgaagaaggcgacctcgtcatctgtcccgaaggtactacctgccgagaacccttccttctccgattttcggccctgtttgccgagctcactgatcgtattgtgcccgttgcaattaataccaagcaaacggtgttccacggcacaaccgtcagaggccataagctgcttgatccttactttgtcttcatgaatcctatgcccacttacgagattacattcctcaaccagcttcccacggaactgacatgtaagggaggaaagtcggccatcgaagttgctaattatatccaacgtgttctcgcaggaactcttggctttgaatgcactaaccttacacgtaaagataagtacgcaatgatggctggaaccgatggccgagtcccttcgaaaaaagaaaaggagcaagagaaggagaaggcataa

SEQ ID NO: 164 - DNA, 1596 bp, Artificial Sequence
  codon optimised ORF
atggccggcaaaccccagaatctcaatgctttcctcttcctccagaagattctcctgaccaaaacttggtcgaaaatttaccttcagcgaaaggcaagcaatagccacggattccaacctcagttcaacaaatatccctttcttgctaatggtctggagactcactcggacaaagcactcatgtttcacacggagggtgctctccttaaatcttcgagcgtgttttcttacttctttcttgtcgccttcgaagctggcggcctcctgcgagccttcatcctttttctgctgcaccccattatctgcctggtgggcaaggagtggggtcttaaaattatggtcttcgtgtgtttcgtcggcctgaaggaggaaagcttcagaattggtcggactgttcttcccaagtttttccttgaagatgttggcaatgagggtttcgatatggtgatgaaatgtggcggtaagaagattggtgtgaccgatatgcctcgtgtgatggtcgattgtttcctcaaagactatctgcgtgtggaggccgtggtgggcagagagctgaaagtggtgtgtggccactttgtcggtcttatggaagaaaaaaaacccagcggtaccgcctttctcgaactggacgaactcctgggcaaagagaacatggatagctctgttattggtatctcgtgttttgataattccctcgatcagcagctcttttcctgctgcgaggttctgtacatggtttctgaggcagacaagaagaattggcaggccctgcctcgtgagaagtaccctagacccctgatttttcatgacggacgactcgcttttagacctacacccctcgcctctcttagaatgtttatgtggatccccttcggcatctttcttcttatcgttcgtgcaaccgttggttatcttatcccttacaagttctcgactccctttctgtgttttactggtctgacaggcacactcattcggcctagctcgtttcccgatcgggctgacgacgaacagaaacaaggcggcgtgctgtacgtctgtaatcaccgaacccttctggatcccatttacgtctccattgtcctggataagccctttaatgcagtcacgtactctctcagccgaatgacagaactgctcgcccccatcaaaacagtccggcttactagaaaccgtgaggaagactccaaaatgattggtaagcttctgaaacaaggcgacctggtgatttgccccgaaggtacgacctgtcgagaaccttaccttctccgtttctcccccctttttgccgaaattacagacgagatcgttcctgttgcaattgacttccaagtcagcatgttttatggcactacagcctccggtttcaagttcctggatcctttctttctccttatgaatcctacctcgcactgttctgcctctattctcgagaaactccccaaatccctgacatgccagggtggaggcaagtcgaaatttgaggtcgcaaaccgtgtgcaggccattatcgccaaagccctgaattttgaatgcacttcgctcacgagaagagataagtacatgattcttgccggtaaccagggaatcgtttaa

SEQ ID NO: 165 - DNA, 1662 bp, Artificial Sequence
  codon optimised ORF
atggcaaaccgacagtccgtgctttggggactcaagacggtttgtctcgtccatcagcacctcaattctaccatctatggcaatctgtgccaacaggccatcaaagtcattaaaaacgatgacgttgcctctggtattaatgaacctcttagcattcttaatcagcgaaagctcccttatctgcgatttaaagtctctaacggtcctgctacgcaattcaaattcctgaaacactctactctcgtgcatagaactgacgagcttgctaatcaaaccctcgcctttcacctggagggcgcccttctcaaaagctgttctctgttcccctatttcatgcttatggcctttgaggctggtggacttttccgagcccttattcttctccttctgtacccccttgtcctcctgattggaaaggaactgggcctgcggattctcgtcttcgtctccttcgtgggcatcagaaaggagaaattcagagctggtacagctattctgcctaaattctttctcgaggacgtgggatgtgagggttttgacatcgttatgtcgtacaaaaagagagtggccgttactggaatgcccaaatttatggttgagggattccttcgagattatctcggtattgatgccgttgttgcacgtgaactgaaagagttccgtggctacttcctgggcctgatggaggagaagatggatgccggtcttgttatcagcgagtgtacacatcatattggcctcggatgctttagaaaatcgcatgaccaaaagattttctctcattgtaaagaaatttatctggttactgaagcagagaagaagaactggcaagtgctgccccggaagagataccctaagcctcttatttttcatgacggcagactcgccttccgacccacgcctctcgcagctcttatcatgttcatttggcttccttttggatttctgctgaacattacccgaaccatcgtcttcatttccctcccttttaagctctctatccccctgctcgccctcagcggatgcatcattaccgtctcgaagcctgagcctacgactgcaagcattaacggtgagaacaagcctggaggaatgctgtacgtctgtaatcatagaacacttctggatcctctcttcctttccgcagttctgatgaagtctgtgtgtgccgtgacctattccatttcccggttctcggaagttatctctcccattaaaactgtgcggctgtcccgtgacagagagaaggacggtaagatcatgaaaaaactcctgtctcaaggtgacctggttgtgtgtcccgagggaacaacatgtcgagagccttatctcctgcggttttcccccctttttgccgagatgacggatgaaattgtccccgtcgcaatcaagctgcaagttagcctgttctacggctccacggctagcggactgaagtgcctcgactctactttccatctgatgaaccccaatcccatgtgctccgtcaaaattctgaataaactgccttcgtgccaaacccacaatacgggaggtaagagcaaatttgaagttaccaaccacgtccagaatcagatcgcagcagcactgggcttcgaatgtactaatctcacgcgaaaagataaatatgctattctcgctggaaatgaaggtattgtgtaa

SEQ ID NO: 166 - DNA, 1386 bp, Artificial Sequence
  codon optimised ORF
atgagcacgctgcctcttcccttcttctctggaacctcgacacgtgctcccttttcgttctctcttaagtcttctcctcctagcgctgccagctctagctcttcttcttctctcgtgccctttcaacgagtggaactgtttagatcgacatcgacgagaaccacacggtcgttttcgtgtgtcttctattccctcaaagctaaggctatggcagaactcgtgcaagataaagaaagcggtgtcgccgccaccggttcctccggtggaggtcgtcctgaggtggagcacagccgaacctttctcgaggcacgaagcgaacaggaacttctttccggcattcgaaaagaagtcgaagcaggtagacttccccctaatattgctgcaggtatggaagaactctaccaaaattaccgaaacgctgttttccaaagcggtgatcccagcgcagtcgagattgtcctttctaatatggccgtggcattcgacagaatgctgctcgatgtggaggaccctttcgttttcgaaccctaccacaaggcccttcgggagcccttcgattactatatgttcggtcagaattacatcagacctctgacggactttcggaactcgtatgtcggcaatctgtctctcttttatgaaatcgaagagaagctgaagcagggccataacgtggtcctcatctctaaccaccaaacagaggccgaccccgctattatcacactcctcctggagaaaacaaacccccacgtcgcagaaaacatgatttatgtcgccggtgatcgtgtgattaccgatcccctttgcaaacccttttctatgggaagaaacctcatctgcgtttactccaagaagcacatgtacgatgtccccgagctggtcgagatgaagcgtaaggctaacacgcaatctcttaagcagatggctcttctcctgcgaggcggttctaaaatcgtttggatcgccccttctggaggtagagatcgtcccgaccctctcactgaagaatggtatcccgctcctttcgattcctcgtccgtcgacaatatgcggcggcttatcgagcacagcggcgctcctggccacatttacccccttgcactcctgtgttataacatcatgccccctcctcctcaagttgagaagcagattggtgaacggagaaacattagcttccacggtgctggactctccgttgctcccaagattagcttccaggagattgcagctgcttgcgagaaatccgaagaagcaaaagatgtctactcgcaagcactgtacaaaagcgttacaaaacaatacaacgttctgaaatcggcagtccatggtaaacagggtctggaggcttcgacagctggcgtttctcttagccagccctggaactaa

SEQ ID NO: 167 - DNA, 1569 bp, Artificial Sequence
  codon optimised ORF
atggctaaaattaaaaaactcttccccctcaaggcactctctctcctcttcgagactctccttaaatcctctggaaaactgccttacctccgtttcaaggtcagcaatggccccgccacgcaattcaaatttctcaagcacagcacgcttgttcaccgtaccgacgaactcgctaaccaaacacttgtttttcatcttgaaggcgctcttctgaaatcctgttcgctgtttccctattttatgctggttgcttttgaagctggcggccttttccgggccctcatcctgcttcttctgtatcctctggtcctgctcattggtaaggagcttggcctgcggatcctggtgttcgtctccttcgtcggaatcacaaaggaaaaattccgagcaggtacgactatccttcccaatttttttctggaggacgtgggctgtgagggatttgacattgttatgagctataaaaaacgagtggcagtcacgggaatgcctaaattcatgattgaaggatttctcagagattatcttggcattgatgccgtggtcgccagagaacttaaggagtttcgtggctactttcttggcctggtcgaagaaaatatggacgccggactggtgatctccgagtgtacgcacaatatcggactcggatgttttcggaagtctcacgatcagaaaattttctcgcattgtaaggagatctatctggtcactgaggctgaaaaaaagaattggcaggtcctccctcggaagcggtatcctaaacccctgatcttccacgatggaagactcgcctttcgtcctacaccctttgctgccctcattatgtttatctggctgcccttcggatttctgcttaacatcactcgggccattatctttattagcctcccttttaacctctctacgcctctgcttgctctctccggtttcattgtgaccgtctctaagcctgaacccactactgcttccattaatggtgagaataagcctggcggtatgctttacgtgtgcaatcaccggacgctccttgaccccctgttcctttccggtgttctgatgaactccgtttcggctgtgacatactcgattagcaagttctctgaggttatttcccctatcaaaactgttggtctttcgcgggacagagaaaaagatggaaaaatcatgaagaaactcctctcgcagggtgacctggtcgtttgtcctgaaggcactacctgtagagaaccctaccttctgcggttctcgcctctcttcgccgaaatgactaatgagattgtgcctgtcgcaatcaagctccaagtttctctcttctacggttccactgcttcgggacttaagtgtctggattctaccttccacctgatgaatcccaaccctatgtgttccgttaaaattctgaacaaacttccctcgtgccagacgcacaacacgggaggcaagagcaaattcgaggtgaacaatcatgtccaaaaccagatcgccgctgcccttggattcgaatgcacgaaccttacgcgtaaagacaaatacgcaattcttgctggaaacgagggtatcgtgtaa

SEQ ID NO: 168 - DNA, 1113 bp, Artificial Sequence
  codon optimised ORF
atggctaatctgcgtgctcacgtttgttttctcctgtcgtttgttactttcctgaatctgatctcctgccatactaatcttcctgcaaaccacgttgctctcttcgtgttcggagattccctgttcgaccctggtaataataactacatcaacacaacctttgattttcaggcaaatttccgtccttacggtgagacgttcttcaagtaccccacaggtcggttcagcgatggtagactcattcctgactttcttgcacagttcgcaggactccctattatccccacttaccttcaacctggcaatcataaattcacggatggtgtcaactttgcatctggcggcgccggcgccctcgtggagtctcaccaaggatttgttgtcgaccttgagactcaaattagatacttcaaaaaagttgaaaaaagccttcgtcaagagctcggcgacgaggaggccaaaaagcttctcagccgtgcagtctatctcatctcggttggaggtaatgattacctcacacggaactcgtctgcatccgatgaggagtatgcctcgatggttcttggtaatcttactgttgcacttaaggagatctacaaaaagggaggaagaaaatttggtttccccaatatgatgcctcttggttgtcttccctatatgaaagcaaaagccggcggaccttgtattgacgagtttacagccattgctaagctccacaataaagaactccccaaaacgctccagaaacttgagactcaacttgagggctttaaatacgcatactataatttttacaaatccgttagcgaacggctcaataatccctctaagtatggcttcaaggatgccaccaccgcatgctgtggaagcggactttacggcggtgtctattcgtgtggtggaaaacgaggaatcacggagtttcacctttgtgagaacccttcggaatacctctttttcgactcttatcaccctagcgaaaaagcttaccagcaattcgcagagcttatgtggagcggcacaacggacttcgtctggccctataatcttaaaactcttttcgaggctaaaaccagcaacctgtatgaccacttcatcattccccccccttaa

SEQ ID NO: 169 - DNA, 1197 bp, Artificial Sequence
  codon optimised ORF
atgcttacgtaccttcctctgagatgtctgggtcttaaatctctcgtttgcatctcttttgattgtggagttattggtgctatggctgctcgagtcaaatattcgtggtccctggccctcgtgctcctgtgtattgtgagccttagctattttggaaacgccgctgacctgggagcttctttcgtttttggcgactctctggttgatgccggaaacaataactacctccctacgctgtccaaagctaatattccccccaacggtatcgactttaaggcaagcggcggacaacctacaggtcgatataccaatggcagaaccatcggagacatcgtcggagaggaactcggcgtccccaactacgctgttcccttcctttctcctaattccacgggaaaggctatcctctacggagtgaattatgcatcgggaggaggtggcatcatgaatgcaactggaagaatctttgtcaatagactcggcctggacattcagattgatttctacaacactactagaaaacagttcgataagctgctgggtcccagcaaggccaaagactacattagaaagtcttccatctttagcattacgattggtgcaaatgattttctgaacaattacctgctccctgtcctttctattggcgcccggattagcgagacacctgatggatttatcgacgatatgatcaaccatcttagagatcagcttacccgtctctatcaactggatgctcggaaattcgtgattggcaacgttggacctatcggatgcatcccctatcagaaaaccatcaatcaactgaatgaaaacgagtgtgtcgaccttgcaaacaagctggccaaacagtataacagccgtcttcagaatctccttacggaacttaatggcaaactgcctggagcaacatttgtcgttgctaacgtgtacgaccttgttatggaactgattaccaattatgaaaaatatggcttcatcacagcctctaaagcttgttgtggtaacggtggccaattcgccggtatcatcccctgcggacccacatcgacgatgtgcaaagatcgagacaagcacgtgttttgggacccctatcatcctagcgaggcagctaatctgattattgccagacaactcctttatggtagcactaaatatatcagccccgtcaacctgaaagagctccgaaacctgtaa

SEQ ID NO: 170 - DNA, 1053 bp, Artificial Sequence
  codon optimised ORF
atgagagttagctcttcgttcctggcttcgctcgtgcttgttctcgttttctctgcagccaacggcgaccctcttgtccctgcccttatttcctttggtgattccgtcgtcgatgtgggcaacaacaataacctcaacacgctcatcaaagccaatttccctccctatggccgtgacttcgttacccaccggcctacaggacgtttttgtaatggtaaacttgccacggactttactgcagagtacctgggctttacctcttaccctcccgcatacctttcccaggatgcacgaggtaatgaccttcttacaggagcaagcttcgcttccgctgcctctggcctttatgatagaaccgctaacctttgccgagctattccccttacacgacaagtcaactattacaaagagtatcagacaaaggtcgtgaatacggttggaaaagccaaggcaaatgatatctttgcaggagcaattcacctcctgagcgctggctctagcgattatatccaaaactactacatcaaccccatgctgaatcgagtttatactcccgaccagttctccgatattctgattcgatcctactctaccttcattcaaaacctttacggactcggtgctcgtcggatcggcgttaccacccttccccccactggatgccttcccgccgctatcacgcttttcggcggaggctctaaccagtgtgtcactcggctcaatcaagacgcagtcgctttcaataagaagcttaataatacttctgagtcgctcctcgacaaaatgcccggacttaagcttctcgttttcgatatctatcaacccctcctcgatatggttatgaagcctgcagacaatggtttcttcgagtctcggcgtgcctgttgcggtactggaacgcttgaaacatcccttctttgtaatgaccgttctcttggcacatgcactaatgctacaagctatgttttctgggatggattccatcctagcgaggccgcaaaccaggtcctcgcaggaaatctgctcgcacagggagtgtcgctcatttcgtaa

SEQ ID NO: 171 - DNA, 1167 bp, Artificial Sequence
  codon optimised ORF
atggccaaactgatctcttacattaacttctttaaagtgtcggttatccttctgctttctacaacatcgcgatgctttggtactcccctggagaaaggttccgctcttttcatcttcggcgatagcacggtcgaccctggcaataataattacatcaacactatccctgagaaccgtgctcattacaaaccttatggacagaacggcttttttgccgagcctactggacgtttttccgacggacgtgtgattgttgactatgtcgcagaatatgcaaagctcccccttatccctcccttcctccaaccctccgctgacttcacaagcggtgcaaatttcgctagcggaggtggtggcgttctccccgaaacaaaccagggactcgtcattgatcttcccacgcagctgaagaattttgaagaggttcagaagagccttgtggaaaaactcggcgaagcacagacgaaggaacttatttcggaggctgtgtactttatttccatcggttcgaacgactacatgggtggttatctcggaaaccccaaaatgcaagagttctaccctcctgaagcctatgttggtatggttatcggaaaccttacacagactctgcaagtcctgtacgagaagggcgctcgtaaattcggctttcttagcctttcgcccctcggatgcctccccgccctgagagccctgaacttcaaggacaatgaaggaaagtgtcttgaagctgcaagcgccctggctcgagcccataatgatgccctcaatgctgttctgacgaacctggaacacatcctccctggccttaaatattgtaattcgaatttctatgaatggctcgacgatcgtatgaacaaccccagcaaatatggatttaaggacggagtgaacgcttgttgcggcacgggtccttacggaggcgtcttcacctgtggaggcacaaagaatgtgaaggattatcagctttgcgacaatgccgacgatcatgtgtggtgggactctttccaccctactgaacggatccacgaacaatttgccaaagcactttggtccggacatccttcttctgttggtccttacaacctcgaggatcttttcttcaataaggagaaactgacaatcgcagatatcgtcgactctcctgaaacagatcccttccaataa

SEQ ID NO: 172 - DNA, 1083 bp, Artificial Sequence
  codon optimised ORF
atgggaaaactctatacaattattgttgtcattctgatcttttcgtccacgctcgcttctagccgtaacgttcccgctatctttatttttggcgacagcatttttgacgccggtaacaaccacttcaataaaaattgctccgtgcaggccgacttctcgccctacggatctacgttcttccatcagcctacgggccggttcacaaatggacgaactgtggccgattttatctctcaatttatcggtatcgatatgcaaaaaccttacctggaagcccagattgcagtcatcaatggatctcgtaaggactatcccagcaatggtatcaattttgcctccgccggaagcggagttctgcagggaacaaacatggagcagggagttatcagcatccaagaacagctgcgtcagtttcaaacgctcgtcgaacaaaaccaaattgacaagaatctggttcagaaatcgctgttctttctcgaaagcggatccaacgacatcttcaattattttatcccctttgaccctcctaccctggaccctgacgcatacgtgcaagctatgatgaaagaggtggcaactcttatcgatcagatctataagctgggagccagaagaattgcagtcttcagccttggacccgttggatgcgtgcctgctcgtgcccttctgcttggcgctcctatcaaccgatgttatggaaagatgaacgtgatggttaaaaaatacaataaaggactcgaatcgcttattaacaacattcctatcagatatcccggcgcaattggtgtttatggcgcaatctacgacattgttcagcgttttcgtgcaattcccgctcggtatggcttctctgacgtcaccagcgcctgctgcggaaatggccctctgagaggaaaactccagtgcggtatcgagggataccagatttgcctgaatcccaatgaagttctcttctgggactatttccaccctacagaacatgcatataaactcatctctgaagccctttggggaggcaacaaatcccagattcgtcccgttaatctgaagaatctggccgatatcaccttcacgcttgtgtaa

SEQ ID NO: 173 - DNA, 8397 bp, Artificial Sequence
  DNA construct
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtggtctccgctagtgctacccatggggtgtctccagaacccttgtacatcagcctatatgcgtgtgtgtctacgtctactgtacaaatctgcttggcgtcaaatggcactacaagtacaaagaccaaatgccacaagttcagtacgagtacaagagtctgctacttgtagctcccgcaggtatttagttcatacaaacgagtatacttgtagctgtccaacagtagttttcagacacctgatcagaaataggaacacgctccacggacctcgtatccactctaagaccataatatatgggtttctggaacattggtgcccctcacctctcatctcttttttttttacctctctgaccaccaccacccttgttgttccgagtggttagctgcaccccacctagcaaactttagggccccatacgcataagcatacgtccggtgtgcgacttgtgtgtgaacgcggtccacacaaggtgtctttcaagtccacacagggctttgtctagagcctgggctatcttcccacgtttgtatatatagactggatccacacccagcaacgcaacgtcctcccacccaaccctatcctccacgttacacgcccaagatcttccaaacaagaagacacattccacagcaagaataaccttggcaagaacgtgcgacgaggtgaataaaagagcccagagtactaacattttgtggacctgttggaaactaattgccgccgcaactcgactggaaccaagttcaagaccactgacaagcgccccggtccaaccagaacactaaacggagccgcacagcaagcaccacggacgatcgtcatcatccctatggccaacctgcatggatactcttgataacagttaagctatatttaccaacaccacctccgttcacgacataacatccccacaccatagctacacccaaagtcgctgtacccgtaccgcccgcaaaatggaagtccgacgacgaaaaatcgacgtgctcaaggcccagaggcccccccggcccccaaatttaaagggggcctttttggccataacttcgtataatgtacattatacgaagttatgaattctaggttagggataacagggtaatgataacttcgtatagcatacattatacgaagttatgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcacaatgaagaagcccgagctgaccgctacctctgttgagaagttcctgattgagaagtttgattccgtttccgacctgatgcagctgtccgagggcgaggagtctcgagccttctcctttgacgtgggcggacgaggttacgttctgcgagtgaactcgtgtgccgacggcttctacaaggatcgatacgtctaccgacactttgcttctgccgctctgcccatccctgaggttctcgacattggcgagttctctgagtccctcacctactgcatctctcgacgagctcagggagtcaccctgcaggacctccctgagactgagctgcctgctgtcctccagcctgttgctgaggccatggacgctatcgctgctgctgatctgtcccagacctcgggtttcggcccctttggacctcagggaattggacagtacaccacttggcgagacttcatctgtgctattgccgatcctcacgtctaccattggcagaccgttatggacgatactgtgtcggcttctgtcgctcaggctctggacgagctgatgctctgggccgaggattgccccgaggttcgacacctggtgcatgctgacttcggttccaacaacgttctcaccgacaacggccgaatcactgccgtgattgactggtccgaggctatgtttggcgactcgcagtacgaggtggccaacatcttcttttggcgaccctggctggcttgtatggagcagcagacccgatacttcgagcgacgacatcctgagctcgctggctcccctcgactgcgagcttacatgctccgaattggtctggaccagctctaccagtcgctggtggatggcaactttgacgatgctgcctgggctcagggacgatgtgacgccatcgtgcgatctggcgctggaaccgtcggacgaactcagattgcccgacgatccgctgctgtctggaccgacggatgcgtggaggtcctggctgattcgggtaaccgacgaccctctactcgacctcgagctaaggagtaagtgtctgtggtatctaagctatttatcactctttacaacttctacctcaactatctactttaataaatgaatatcgtttattctctatgattactgtatatgcgttcctctaagacaaatcgaatataacttcgtatagcaggagttatccgaagcgagtagggataacagggtaatacggagaattcgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtcagctcgggtgaggacattttcagcggactcgtccccattctgattgaactcgagggtgacgttaatggacaccgtttctctgtccgaggtgaaggttacggcgacgcttctaacggcaaactggaaatcaagttcatctgcacgacaggacggcttcccgtgccctggcctactctggtcacaaccctctcctatggagtgcaatgttttgctaagtaccctgagcacatgcggcaaaatgatttttttaagagcgccatgcctgatggatacgtccaagaacgaaccatctcctttaaagaagacggaacgtataaaacacgagccgaagtcaaatttgaaggcgaagctctcgtcaatcgaattgatctgaaaggcctggagtttaaggaggacggcaacatcctcggacataaactcgagtacagctttaattcccattacgtttacattacagcagataagaatagaaatggactggaggctcagtttcggattcgtcataacgtggacgatggaagcgtccaacttgcagaccattatcaacagaatacacctatcggcgaaggccccgtccttctccccgaacagcattacctgactaccaattccgtccttagcaaagatcctcaggaaagacgagatcacatggtgctggttgagtttgttaccgctgccggactcagcctgggaatggatgagctctataaatcctaagtgtctgtggtatctaagctatttatcactctttacaacttctacctcaactatctactttaataaatgaatatcgtttattctctatgattactgtatatgcgttcctctaagacaataacttcgtataatgtacattatacgaagttataacccacttgtgcattcctttattatctggcttacaactacaagcagaaccagtagcataacactcatcagtagcctttacagtgataaataatattgacattgacgtaatctgatgattgtagacaaacgtgagacggcattagcgataatattttgacgatgcccacgggcatataataaccattctgtaatagaactttgagcatgttcagaagtaatgagtttcatacataggagtacagatcgtggcgatgtgtaattaccggtaccgcatatgttgaacctgttcgtaaaaggtgccttcaacacagttttgttgccgtcttacctgtgtataagcgttcagcttctggtgtgtagcagaaggaggtgaagttgaggaacaaataatggagacttgcagcagactccgtgcctcaagcaaaaaagatgatccagcagaggatcgaactctgagcctcctcggcactcgtagattgaagccactttgtgagcgagacgtgataaccattacaccactagaccgggggtgaagatcagagcccgtattgtcgtattcaaagttaaaaatggggattttttatattcagaagtgtgtgatttattgaacgccaataacccggcttactacagtaggtatatagctgtaatgaatcattagaataaaaaaaaatccgcctcaaagtagctgaaatgtatccctagaataatgaggggaaaaaactgataaatcaggtgaaatacggcatttagggacaggtaagagttcaattgaatttatgtcgtattatttaagctgtcggagaaaacttgtgtgcttttttgtatggttttcgtgactgtgtacatttctgtacccctgattccgaacatcttatactgacacaagacattggagataaatcatacagtataaagtagctacgtcttacttgtacagtgcttttaattacaatatgccccactatacatacatgtgattcattttataaacccctgagacccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 174 - DNA, 7984 bp, Artificial Sequence
  DNA construct
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtggtctctgtaacctcggcaggaggagatcggcaaagctgaagatgattgatgcatgtggcgtttttgaaggaactaccggcagggtttgttggtgttaatgtgaacacagctttttttttgcttaataagatctccaaacacgttcaaaaaacacccactagttaaaagctcagttggactacctattgttccttctaatatacagtagactgctcgtacagtagtctctctaatcgatcggtgtctcaatgtctcaccgttaccggatacgcttatggtcgattgaattgccaatggaattatcggtggaaattcatggaaaaccggtttcggcgagctcgaaatacaccccgctatcgtacaagtcgaacgaatatcaggcgcccagacagatcgattcaagtagggagaagagtggaacaacttgtaaagaatgactgatggtgacgctcgggcccaggctagcactgaccatcaaatccatcttatcgatctgcttgaatctacaagtaggagggttggagtgattaagtgaaacttctttaacggctctatgccagttctattgatatccgaaacatcagtatgaaggtctgataagggtgacttcttcccacagattcgtatcagtacgagtacgagtaccggtacttgtaacagtattgatactaaagggaaactacaacggttgtcagcgtaatgtgacttcgcccatgaacgcagacacgcagtgccgagtgcggtgatatcgcctactcgttacgtccatggactacacaacccctcggcttcgcttggcttagcctcgggctcggtgctgttcagttaaaacacaatcaaataacatttctagctctactttttagaaggcaggccgtcaggagcaactccgactccattgacgtttctaaacatctgaatgccttccttaccttcaacaaactggcaggttcgggcgacagtgtaaagagacttgatgaagttggtgtcgtcgtgtcggtagtgggcccccccggcccccaaatttaaagggggcctttttggccataacttcgtataatgtacattatacgaagttattctgaattccgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcgataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaacgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagtgctggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgcgaattcgtgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtcagctcgggtgaggacattttcagcggactcgtccccattctgattgaactcgagggtgacgttaatggacaccgtttctctgtccgaggtgaaggttacggcgacgcttctaacggcaaactggaaatcaagttcatctgcacgacaggacggcttcccgtgccctggcctactctggtcacaaccctctcctatggagtgcaatgttttgctaagtaccctgagcacatgcggcaaaatgatttttttaagagcgccatgcctgatggatacgtccaagaacgaaccatctcctttaaagaagacggaacgtataaaacacgagccgaagtcaaatttgaaggcgaagctctcgtcaatcgaattgatctgaaaggcctggagtttaaggaggacggcaacatcctcggacataaactcgagtacagctttaattcccattacgtttacattacagcagataagaatagaaatggactggaggctcagtttcggattcgtcataacgtggacgatggaagcgtccaacttgcagaccattatcaacagaatacacctatcggcgaaggccccgtccttctccccgaacagcattacctgactaccaattccgtccttagcaaagatcctcaggaaagacgagatcacatggtgctggttgagtttgttaccgctgccggactcagcctgggaatggatgagctctataaatcctaagtgtctgtggtatctaagctatttatcactctttacaacttctacctcaactatctactttaataaatgaatatcgtttattctctatgattactgtatatgcgttcctctaagacaataacttcgtataatgtacattatacgaagttatctgcacggccaccttgccggtgccggtgttggtctgcgtggtcgaatccatttctatggtcggtctgttggaatacgagaaagggactcaggatgccgaccggtatatatttcaatgtcttacctcgagtttaaacccactaggcttttttttttctccctgtcaatccaccaacctttttggcctggcgtgactgacttggggacaagcaaaagctgagggtaaacagaggcacacccgaggttatatgttgagggccaatgtcaaattttcctcctaatcctgagagcccctctaacaccagcacgcatctacctcttgtatataactacgggccccgcctctcctaaccgccactttaacacgctcgtactcctcgtctgttaccacctgaaaaacgcataatgcgcatgtgcggattggccaagagagctccgaacggaaccgcccggggtatactgctgctctcagcgtggatagtgtggagggaccacgtctcaaccgtgacatgggtcgacttatggcgggggggagagtgaaagaaccagttacacgtgcaggtgaagaggggaagaccttggacgtgttcacgtgactacgtcggttctacatgaatccaacgtctcttatttcgtcatgtgctactacatgacccccattggcacgactctctctctgtgctgccactacttgacgactatgtccgcttgttgactcgttgaacaggtgtccgatgaatagcatggcttctcaacaggccatgcttaaagttagagttgggagatgtcgttccgaaccttgtgggcggcggtgtggaaagatagagagtacaagacaaccaaactcttggagcctttggctcgtaaagagagctcttccggtccatcgacgaagaaacgcagcgttgaaagactgccagaagcggcagtcaccgccgtatctgagacattgatcgtacacactcttgcacaagggctatgcttaactacttgtacgggtagagacccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 175 - DNA, 7942 bp, Artificial Sequence
  DNA construct
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtggtctccagcctcaaaagcacagatttaccccgtttattgcccccaaatcgccccactagcccccaacatcaacgtctcaaaaatacgagcgtcctgtccttgtcaaagagtgtctcacaggctttattgtacgccctacacctgcactttgcatggtgctacgcttaaaatgaaaccctggaatgtgaagcgagatgaggtattttaacgggttggaaaaagaagcaaatggggttgctttttggaggcggatgatagccaacagaaccgaaatggccaaagaagatctcctccaatgggtctttgagcggctcggggtcgcttagaagaccgcaaaacactcttttcagccgctccaagcagtcttgtgaatcttggcgtcaccatgggttggcattggtggcggagtaaaatatattttgttgaggaggtgacaggagtggttgtatattggaagcaattcgttttttgtcgaacattttcaggagtagatttcagtgatttccctgtttcccctcttttctcctgaatttcccgatttattccaaacaattgaaatatattctttatcagaacgcgcagatctggagaaaggcgcatattgtgggggaaacattcttcattttgcgggaaaacacgcgttgatgacgtttgttgctcgttcgttcgtcgttcgtcgttcgtcgttcgtcgttctacgtgctcattaaacaaccacatggaatcatgagaaatcgaacgcccttctccatcagtgtgatatacaaaacatattactctacaaaacggactctctaattgctgccgagatcaatcttccacacccactcatcaatgttggagatgactctctcttcaatctcatttccctttccactagccaccttcagaaccatctcgttcaactcagaacgccccagaatgtccacatgctcggcagtctgagcaccgcctcgaatatcaagtcgatcaggctggtgcaacatctccacaaccttgacctggggcccccccggcccccaaatttaaagggggcctttttggccataacttcgtataatgtacattatacgaagttatgaattctaggttagggataacagggtaatgataacttcgtatagcatacattatacgaagttatggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcacaatgaccactctggatgacaccgcttaccgataccgaacttccgttcctggcgatgccgaggctattgaggctctggatggatctttcaccactgacaccgttttccgagtgaccgctactggcgacggcttcaccctgcgagaggtgcctgtcgaccctcctctcaccaaggttttccctgacgatgagtcggacgatgagtctgacgctggagaggacggcgaccctgactctcgaactttcgtggcttacggcgacgatggcgacctggccggctttgtggtcgtttcttactccggatggaaccgacgactgaccgtggaggacatcgaggtcgctcctgagcaccgaggtcatggtgtcggacgagctctgatgggtctggctactgagttcgctcgagagcgaggtgctggccacctgtggctcgaggtcaccaacgttaacgcccctgctattcatgcctaccgacgaatgggttttaccctgtgtggcctcgatactgccctgtacgacggaaccgcttccgatggagagcaggccctctacatgtcgatgccctgcccttaagtgtctgtggtatctaagctatttatcactctttacaacttctacctcaactatctactttaataaatgaatatcgtttattctctatgattactgtatatgcgttcctctaagacaaatcgaatataacttcgtatagcaggagttatccgaagcgagtagggataacagggtaatacggagaattcgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtcagctcgggtgaggacattttcagcggactcgtccccattctgattgaactcgagggtgacgttaatggacaccgtttctctgtccgaggtgaaggttacggcgacgcttctaacggcaaactggaaatcaagttcatctgcacgacaggacggcttcccgtgccctggcctactctggtcacaaccctctcctatggagtgcaatgttttgctaagtaccctgagcacatgcggcaaaatgatttttttaagagcgccatgcctgatggatacgtccaagaacgaaccatctcctttaaagaagacggaacgtataaaacacgagccgaagtcaaatttgaaggcgaagctctcgtcaatcgaattgatctgaaaggcctggagtttaaggaggacggcaacatcctcggacataaactcgagtacagctttaattcccattacgtttacattacagcagataagaatagaaatggactggaggctcagtttcggattcgtcataacgtggacgatggaagcgtccaacttgcagaccattatcaacagaatacacctatcggcgaaggccccgtccttctccccgaacagcattacctgactaccaattccgtccttagcaaagatcctcaggaaagacgagatcacatggtgctggttgagtttgttaccgctgccggactcagcctgggaatggatgagctctataaatcctaagtgtctgtggtatctaagctatttatcactctttacaacttctacctcaactatctactttaataaatgaatatcgtttattctctatgattactgtatatgcgttcctctaagacaataacttcgtataatgtacattatacgaagttattgtcttgtacaaagtccgacacactgtccatggacaagttgtcgagagatagagcgtcaaagtcgaacgagggcaagtcgccgagaagagcatcgatatcaatgctgagctccggtggcgcagcgtagtatcccgcggctatcacaccgaccacggcgccgaggacgaaggtgatacgtcgacttcgggtgaagaagttgagaatcttgcccttgacggattttcgcttgtggtggtgatgatggtggtgaacgtggacgtcggtcttggcctcggactcggagtcagccacctccactgctggctcgacccgctcgacagtcgccttccgattcacaggttgtgtcattgatggttgtgatcaaccggaaaaaagccgattacactttttttacatgagccagaccggggtagctgagacatgtcgcgtgaatgattagtcatggagcgagctgcgggagacaactgctagaaggataagccggaattgacgttcgtattcaattaaaattcgatactgtggcgattttagcggcgtttgtctgccgggcaaatcgagaatcccactatcatatgcgcgtttcggttattttgctggtgcatgaagagcataatattaataaatgtggggcgaacatgaaaaaagtgcggttggtactggaaatggatgtaaaaacagtcatggtcgaaccaaaagagtggtgccattgcttgtatttctgacttcatctgtacaaagaaatcgcgtcagaatggtcttgtttcagtggaccccgcagaatggtcctgtgtcagtggacaacagcaagacctcatgcaagggtaggtccaagcacagtatcaagctgctgtcgtatcgtacctactagagaactctggacacagcacttgtagcaaaggtcggtcagtactggagcttcggtgccatgggggatacgctgtcagttgtgtggagaagaagcagactccagattaagagaaccattctctaggtcatcgtgagacccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 176 - DNA, 8397 bp, Artificial Sequence
  DNA construct
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgcggccgctgtcgggaaccgcgttcaggtggaacaggaccacctcccttgcacttcttggtatatcagtataggctgatgtattcatagtggggtttttcataataaatttactaacggcaggcaacattcactcggcttaaacgcaaaacggaccgtcttgatatcttctgacgcattgaccaccgagaaatagtgttagttaccgggtgagttattgttcttctacacaggcgacgcccatcgtctagagttgatgtactaactcagatttcactacctaccctatccctggtacgcacaaagcactttgctagaaggtggtctcaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaaccccaacaacactggcaccattgaaatcaacggtaaggagtacaacaccttcaccgagccccccgtggcccccccggcccccaaatttaaagggggcctttttggccataacttcgtataatgtacattatacgaagttatgaattctaggttagggataacagggtaatgataacttcgtatagcatacattatacgaagttatgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcacaatgaagaagcccgagctgaccgctacctctgttgagaagttcctgattgagaagtttgattccgtttccgacctgatgcagctgtccgagggcgaggagtctcgagccttctcctttgacgtgggcggacgaggttacgttctgcgagtgaactcgtgtgccgacggcttctacaaggatcgatacgtctaccgacactttgcttctgccgctctgcccatccctgaggttctcgacattggcgagttctctgagtccctcacctactgcatctctcgacgagctcagggagtcaccctgcaggacctccctgagactgagctgcctgctgtcctccagcctgttgctgaggccatggacgctatcgctgctgctgatctgtcccagacctcgggtttcggcccctttggacctcagggaattggacagtacaccacttggcgagacttcatctgtgctattgccgatcctcacgtctaccattggcagaccgttatggacgatactgtgtcggcttctgtcgctcaggctctggacgagctgatgctctgggccgaggattgccccgaggttcgacacctggtgcatgctgacttcggttccaacaacgttctcaccgacaacggccgaatcactgccgtgattgactggtccgaggctatgtttggcgactcgcagtacgaggtggccaacatcttcttttggcgaccctggctggcttgtatggagcagcagacccgatacttcgagcgacgacatcctgagctcgctggctcccctcgactgcgagcttacatgctccgaattggtctggaccagctctaccagtcgctggtggatggcaactttgacgatgctgcctgggctcagggacgatgtgacgccatcgtgcgatctggcgctggaaccgtcggacgaactcagattgcccgacgatccgctgctgtctggaccgacggatgcgtggaggtcctggctgattcgggtaaccgacgaccctctactcgacctcgagctaaggagtaagtgtctgtggtatctaagctatttatcactctttacaacttctacctcaactatctactttaataaatgaatatcgtttattctctatgattactgtatatgcgttcctctaagacaaatcgaatataacttcgtatagcaggagttatccgaagcgagtagggataacagggtaatacggagaattcgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtcagctcgggtgaggacattttcagcggactcgtccccattctgattgaactcgagggtgacgttaatggacaccgtttctctgtccgaggtgaaggttacggcgacgcttctaacggcaaactggaaatcaagttcatctgcacgacaggacggcttcccgtgccctggcctactctggtcacaaccctctcctatggagtgcaatgttttgctaagtaccctgagcacatgcggcaaaatgatttttttaagagcgccatgcctgatggatacgtccaagaacgaaccatctcctttaaagaagacggaacgtataaaacacgagccgaagtcaaatttgaaggcgaagctctcgtcaatcgaattgatctgaaaggcctggagtttaaggaggacggcaacatcctcggacataaactcgagtacagctttaattcccattacgtttacattacagcagataagaatagaaatggactggaggctcagtttcggattcgtcataacgtggacgatggaagcgtccaacttgcagaccattatcaacagaatacacctatcggcgaaggccccgtccttctccccgaacagcattacctgactaccaattccgtccttagcaaagatcctcaggaaagacgagatcacatggtgctggttgagtttgttaccgctgccggactcagcctgggaatggatgagctctataaatcctaagtgtctgtggtatctaagctatttatcactctttacaacttctacctcaactatctactttaataaatgaatatcgtttattctctatgattactgtatatgcgttcctctaagacaataacttcgtataatgtacattatacgaagttattcaagcccttccttttccgagaggaggaggatgatgacatttgcgagcttgatgaggaatagacaagcgggtatttattgtatgaataaagattatgtattgattgcaaaaaagtgcatttgtagatgtggtttattgtagagagtacggtatgtactgtacgaacattaggagctacttctacaagtagattttcttaacaagggtgaaatttactaggaagtacatgcatatttcgttagtagaatcacaaaagaaatgtacaagcacgtactacttgtactccacaatgtggagtgggagcaaaaaaattggacgacaccggaatcgaaccggggacctcgcgcatgctaagcgcatgtgataaccaactacaccagacgcccaagaactttcttggtgattatggaatacgtggtctgctatatctcaattttgctgtaatgaatcattagaattaaaaaaaaaaccccatttttgtgtgattgtcggccaagagatggaacaggaagaatacgtgaacaagcgagcacgaatgccatatgctcttctgaacaaccgagtccgaatccgatttgtgggtatcacatgtctcaagtagctgaaatgtatttcgctagaataaaataaatgagattaagaattaaaaatattggaatatattttcctagaatagaaactttggattttttttcggctattacagtctgaactggacaaacggctgactatatataaatattattgggtctgttttcttgtttatgtcgaaattatctgggttttactactgtgtcgtcgagtatagagtggcctgactggagaaaatgcagtagtatggacagtaggtactgccagccagagaagtttttggaattgatacttgagtcatttttccattccccattccccattccaacacaatcaactgtttctgaacattttccaaaacgcggagatgtatgtcacttggcactgcaagtctcgattcaaaatgcatgagacccatgtgtaacactcgctctggagagttagtcatccgacagggtaactctaatctcccaacaccttattaactctgcgtaactgtaactcttcttgccacgtcgatcttactcaattttcctgctcatcatctgctggattgttgtctatcgtctggctctaatacatttattgtttattgcccaaacaactttcattgcacgtaagtgaattgttttataacagcgttcgccaattgctgcgccatcgtcgtccggctgtcctaccgttaggggtagtgtgtctcacactaccgaggttactagagttgggaaagcgatactgcctcggacacaccacctgggtcttacgactgcagagagaatcggcgttacctctctcacaaagcccttcagtgcggccgcatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc